(19) United States
(12) Patent Application Publication
(10) Pub. No.: US 2024/0059698 A1
(43) Pub. Date: Feb. 22, 2024

(54) HETEROARYL COMPOUNDS AS EGFR INHIBITORS AND THEIR USES

(71) Applicant: Accutar Biotechnology Inc., Cranbury, NJ (US)

(72) Inventors: Jie FAN, New York, NY (US); Yimin QIAN, Plainsboro, NJ (US); Wei HE, Zionsville, IN (US); Ke LIU, Shanghai (CN); Pin HUANG, Shanghai (CN); Xiangyan SUN, Shanghai (CN); Jie SU, New York, NY (US); Ji LIU, Shanghai (CN)

(73) Assignee: Accutar Biotechnology Inc., Cranbury, NJ (US)

(21) Appl. No.: 18/361,062

(22) Filed: Jul. 28, 2023

Related U.S. Application Data
(60) Provisional application No. 63/393,470, filed on Jul. 29, 2022.

(51) Int. Cl. C07D 487/04; C07D 519/00; C07D 471/04; C07D 495/04; A61K 45/06; A61P 35/00

(52) U.S. Cl. CPC C07D 487/04; C07D 519/00; C07D 471/04; C07D 495/04; A61K 45/06; A61P 35/00; C07B 2200/05

(57) ABSTRACT

Disclosed are heteroaryl compounds and pharmaceutical compositions thereof, which are useful for inhibiting EGFR, as well as methods for using such compounds to treat cancer associated with an EGFR or HER2 exon 20 insertion mutation.

This application claims the benefit of priority to U.S. Provisional Patent Application No. 63/393,470, filed Jul. 29, 2022, which is hereby incorporated by reference in its entirety.

### FIELD OF THE DISCLOSURE

The present invention relates to novel heteroaryl compounds, or pharmaceutically acceptable salts thereof, which possess anti-tumor activity and are accordingly useful in methods of treatment of the human or animal body. For example, the present invention relates to covalent compounds/inhibitors that bind to Epidermal Growth Factor Receptor (EGFR), pharmaceutical compositions comprising the compounds, and methods of use therefor.

### BACKGROUND OF THE DISCLOSURE

Protein kinases are a group of enzymes that regulate diverse, important biological processes including, for example, call growth, proliferation, survival, invasion and differentiation, organ formation, tissue repair and regeneration. Protein kinases exert their physiological functions through catalyzing the phosphorylation of protein and thereby modulating cellular activities. Because protein kinases have profound effects on cells, their activities are highly regulated. Kinases are turned on or off by phosphorylation (sometimes by autophosphorylation), by binding of activator proteins or inhibitor proteins, or small molecules, or by controlling their location in the cell relative to their substrates.

EGFR is a transmembrane protein tyrosine kinase member of the erbB receptor family. Upon binding of a growth factor ligand such as epidermal growth factor (EGF), the receptor can homo-dimerize with another EGFR molecule or hetero-dimerize with another family member such as erbB2 (HER2), erbB3 (HER3), or erbB4 (HER4).

Homo- and/or hetero-dimerization of erbB receptors results in the phosphorylation of key tyrosine residues in the intracellular domain and leads to the stimulation of numerous intracellular signal transduction pathways involved in cell proliferation and survival. Deregulation of erbB family signaling promotes proliferation, invasion, metastasis, angiogenesis, and tumor cell survival and has been described in many human cancers, including those of the lung, head and neck and breast.

The erbB family therefore represents a rational target for anticancer drug development and a number of agents targeting EGFR or erbB2 are now clinically available, including gefitinib (IRESSA®), erlotinib (TARCEVA®) and lapatinb (TYKERB®). Detailed reviews of erbB receptor signaling and its involvement in tumorigenesis are provided in New England Journal of Medicine (2008) Vol. 358, 1160-74 and Biochemical and Biophysical Research Communications (2004) Vol. 319, 1-11.

In 2004 it was reported (Science [2004] Vol. 304, 1497-500 and New England Journal of Medicine [2004] Vol. 350, 2129-39) that activating mutations in EGFR correlated with response to gefitinib therapy in non-small-cell lung cancer (NSCLC). The most common EGFR activating mutations, L858R and delE746_A750, result in an increase in affinity for small molecule tyrosine kinase inhibitors such as gefitinib and erlotinib and a decrease in affinity for adenosine triphosphate (ATP) relative to wild type (WT) EGFR. Ultimately, acquired resistance to therapy with gefitinib or erlotinib arises, for example by mutation of the gatekeeper residue T790M, which is reportedly detected in 50% of clinically resistant patients. This mutation is not believed to hinder the binding of gefitinib or erlotinib to EGFR sterically, merely to after the affinity to ATP to levels comparable to WT EGFR.

In view of the importance of this mutation in resistance to existing therapies targeting EGFR, it is believed that agents which can inhibit EGFR harboring the gatekeeper mutation may be especially useful in the treatment of cancer.

Exon20 insertion mutations represents the third most common erbB family activating mutations in NSCLC. EGFR exon 20 insertion mutations are collectively representing approximately 4% to 10% of all EGFR mutations. Most of EGFR exon 20 insertion mutations occur near the end of αC-helix after residue Met766, with EGFR D770_N771insSVD and V769_D770insASV accounting for about 40% of them. As EGFR exon20 insertion mutations, erbB2 exon20 insertion mutations occur in a similar prevalence in NSCLC and also in a similar position after residue Met774, with erbB2 A775_G776insYVMA accounting for about 80% of them. See, Jang, J. et al. Angew. Chem. Int. Ed. (2018) Vol. 57(36), 11629-11633.

HER2 mutations are reportedly present in about 2-4% of NSCLC (See, Stephens et al. Nature (2004) Vol. 431, 525-526). The most common mutation is an in-frame insertion within exon 20. In 83% of patients having HER2 associated NSCLC, a four amino acid YVMA insertion mutation occurs at codon 775 in exon 20 of HER2. (See, Arcila et al. Clin Cancer Res (2012) Vol. 18, 4910-4918). The exon 20 insertion results in increased HER2 kinase activity and enhanced signaling through downstream pathways, resulting in increased survival, invasiveness, and tumorigenicity (See, Wang et al. Cancer Cell (2006) Vol. 10, 25-38). Tumors harboring the HER2 YVMA mutation are largely resistant to known EGFR inhibitors. (See, Arcila et al. 2012).

Exon 20 insertion mutations are not restricted to lung cancer. Recent analysis of sinonasal squamous cell carcinoma (SNSCC), a rear form of head and neck cancer, demonstrated a remarkably high frequency of EGFR mutations (77% of SNSCC tumors), the majority of which were exon 20 insertions (88% of all EGFR mutations). See, Udager, A. M. et al. Cancer Res. (2015) Vol. 75, 2600-2606.

Exon 20 insertion mutations rarely respond to treatment with currently approved EGFR and HER2 TKIs, such as gefitinib, erlotinib or afatinib, or chemotherapies.

There remains a need for compounds that may exhibit favorable potency profiles against WT EGFR versus activating mutant forms of EGFR/erbB2 (for example the L858R EGFR mutant, or the delE746_A750 mutant or the Exon19 deletion EGFR mutant, or EGFR/erbB2 exon20 insertion mutations) and/or resistant mutant forms of EGFR (for example T790M EGFR mutant), and/or selectivity over other enzyme receptors which may make the compounds especially promising for development as therapeutic agents. In this regard, there remains a need for compounds that show a higher inhibition of certain activating or resistance mutant forms of EGFR and HER2 while at the same time showing relatively low inhibition of WT EGFR. Such compounds may be expected to be more suitable as therapeutic agents, particularly for the treatment of cancer, due to reduction of toxicology associated with WT EGFR inhibition. Such toxicologies are known to manifest themselves in humans as skin rashes and/or diarrhea. The inventors have found novel heteroaryl compounds that have high potency against several mutant forms of EGFR and HER2 while at the same showing relatively low inhibition of WT EGFR.

### SUMMARY OF THE DISCLOSURE

The invention provides compounds, compositions and methods for modulating the activity of EGFR.

In one aspect, the invention provides compounds which act as inhibitors of EGFR.

Disclosed are compounds of Formula (1) or a tautomer, stereoisomer or a mixture of stereoisomers, or a pharmaceutically acceptable salt, or hydrate, or deuterated derivative thereof:

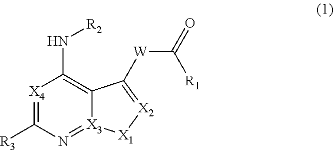

wherein:
X₁ is chosen from NH, N—CH₃, S, and CH;
X₂ is chosen from CH, N, C—CH₃, and N-methylpiperazine-4-phenyl;
X₃ is chosen from C and N;
X₄ is chosen from N, C—CN, and C—F;
R₃ is chosen from H and C₁-C₅ alkyl;
R₁ is chosen from

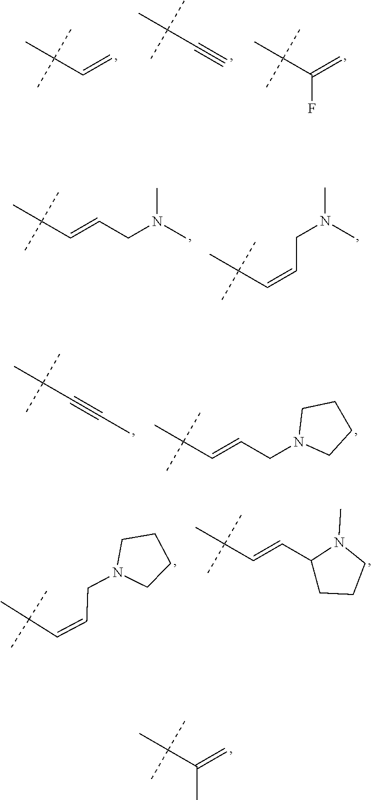

and C₁-C₁₀ alkyl;
W is chosen from alkylamino group, azetidine, methylazetidine, azetidine-3-amine, piperidine, tetrahydropyridine, pyrrolidine, 3-aminopyrrolidine, cyclobutylamine, cyclopentylamine, azepane, bicyclic amine, bridge cyclic amine, a spirocyclic amine, and an aryl amine, each of which is optionally substituted with halogen, hydroxyl, C₁-C₅ alkyl, or C₁-C₅ alkoxy; and
and R₂ is chosen from

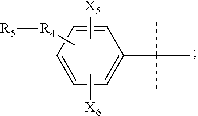

wherein:
X₅ and X₆ are each independently a halogen or a cyano group;
R₄ is chosen from a C₁-C₃ alkyl, a C₁-C₃ alkoxy,

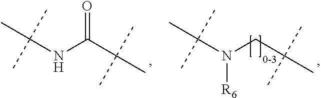

and O; and
R₅ is chosen from optionally substituted aryls and optionally substituted heteroaryls;
wherein R₆ is H or CH₃.

In some embodiments, the compound of Formula (1) may be a compound of Formula (2):

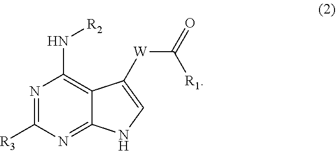

In some embodiments, the compound of Formula (1) may be a compound of Formula (3):

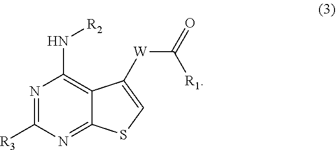

In some embodiments, the compound of Formula (1) may be a compound of Formula (4):

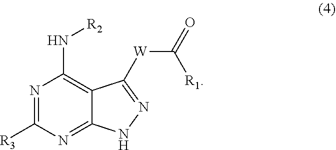

In some embodiments, the compound of Formula (1) may be a compound of Formula (5):

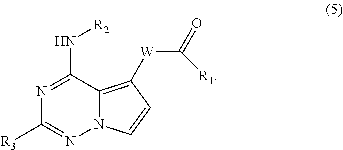

In some embodiments, W is chosen from

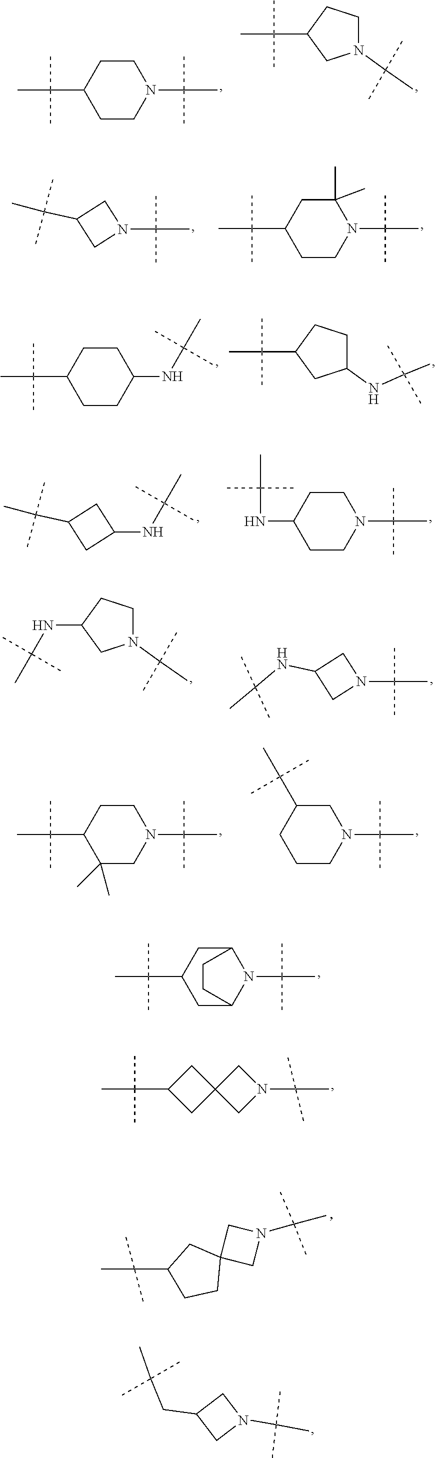

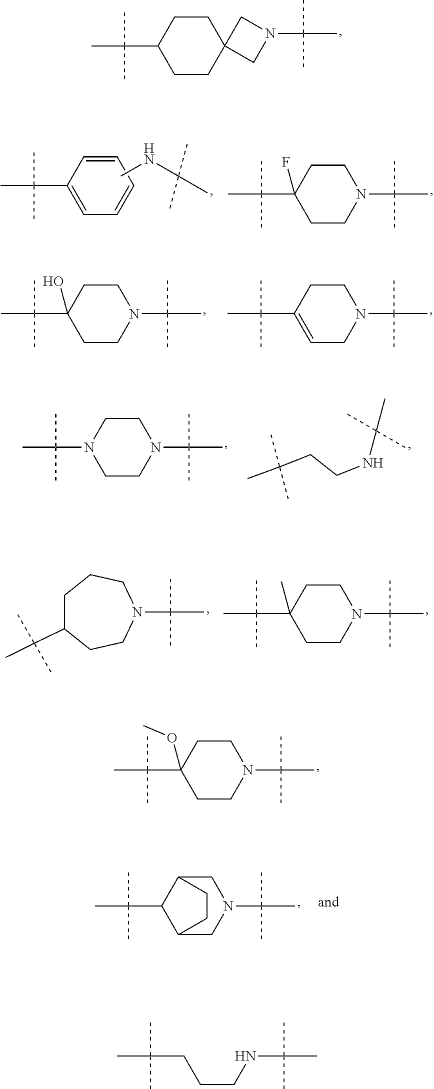

In some embodiments, R₂ is chosen from

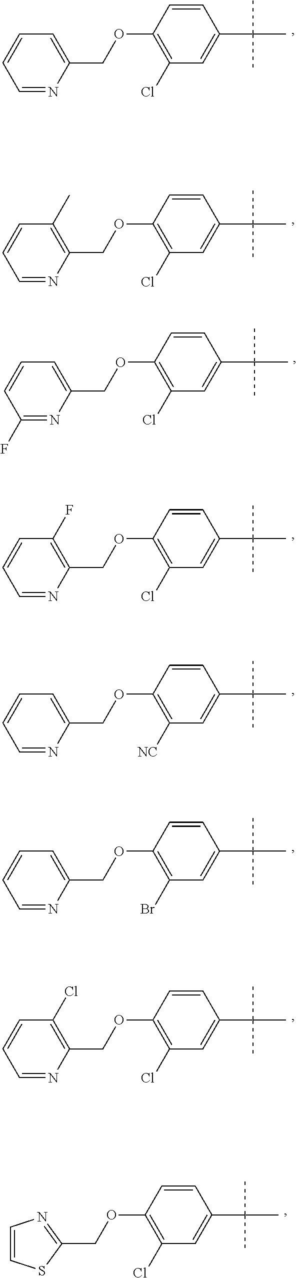

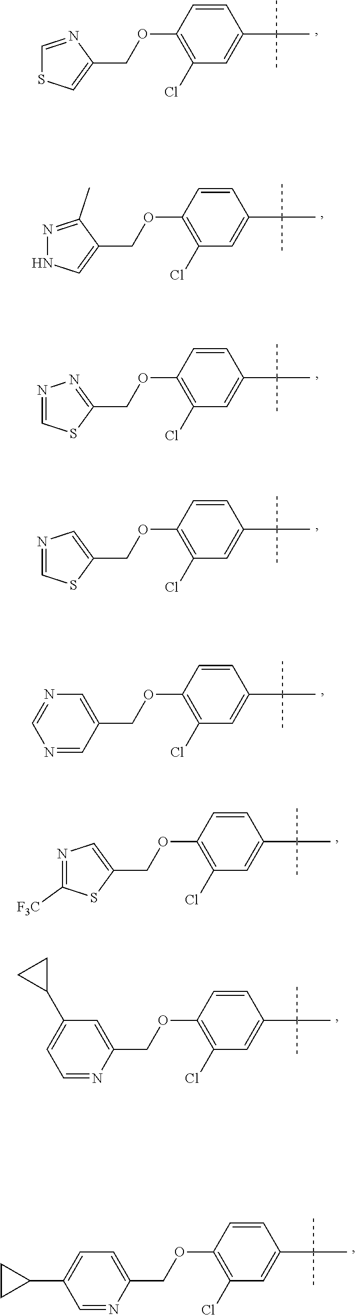

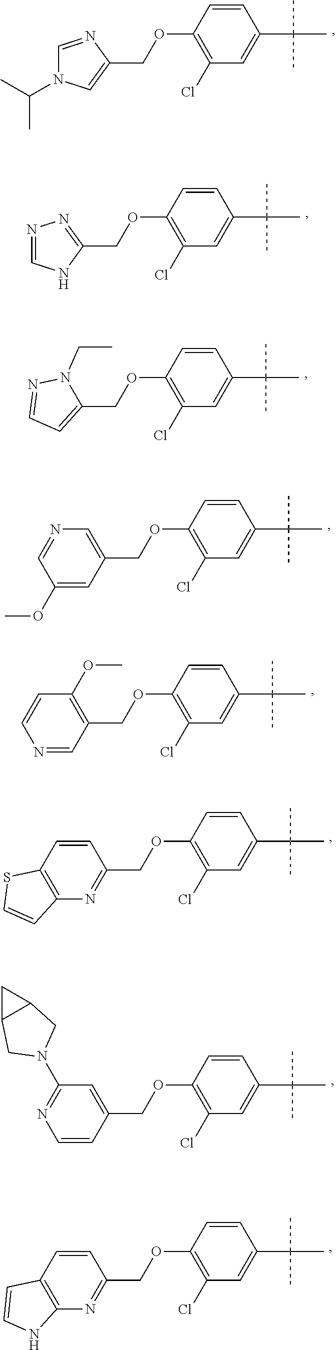

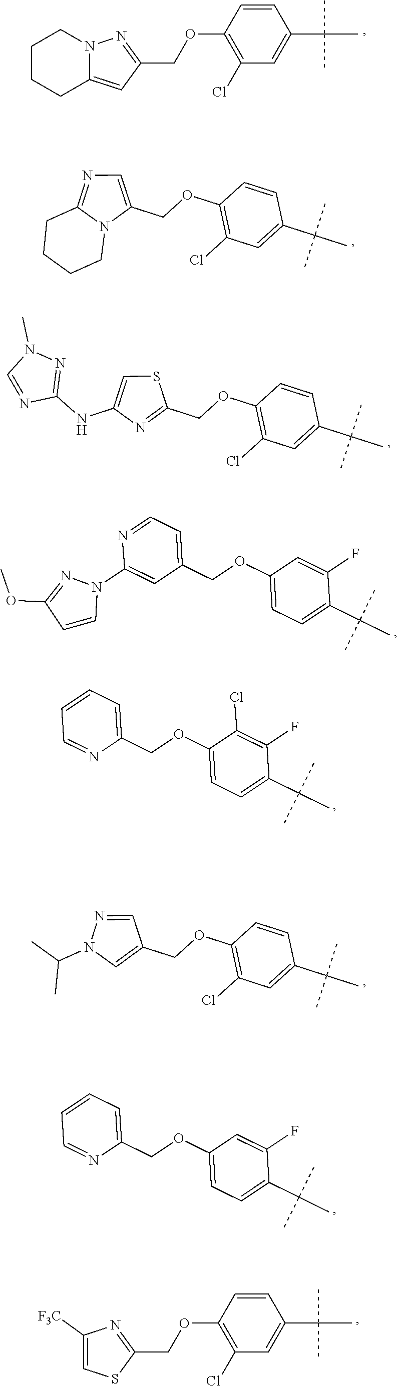

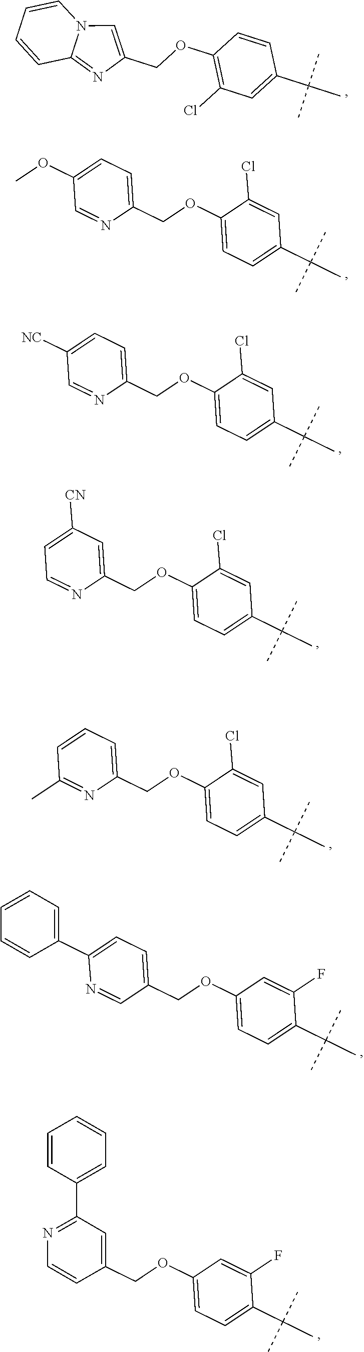

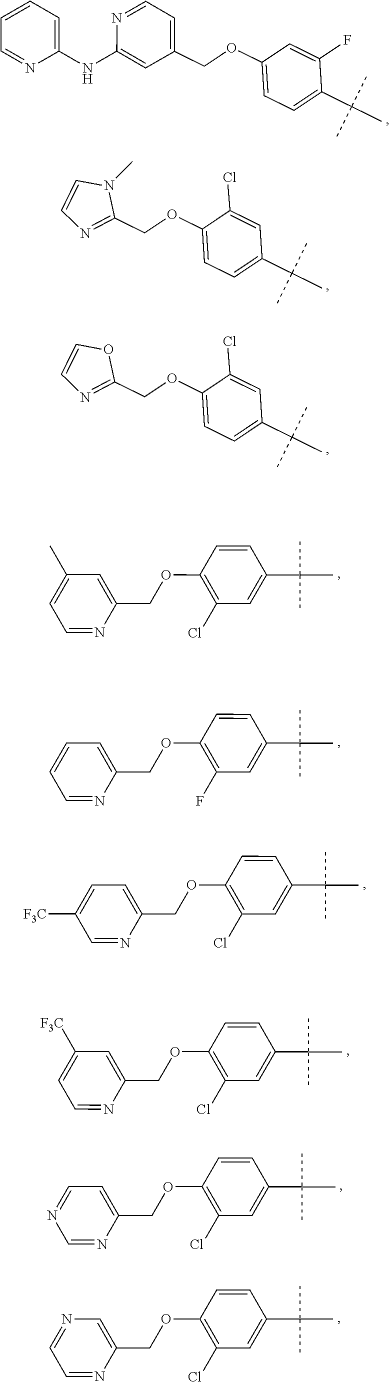

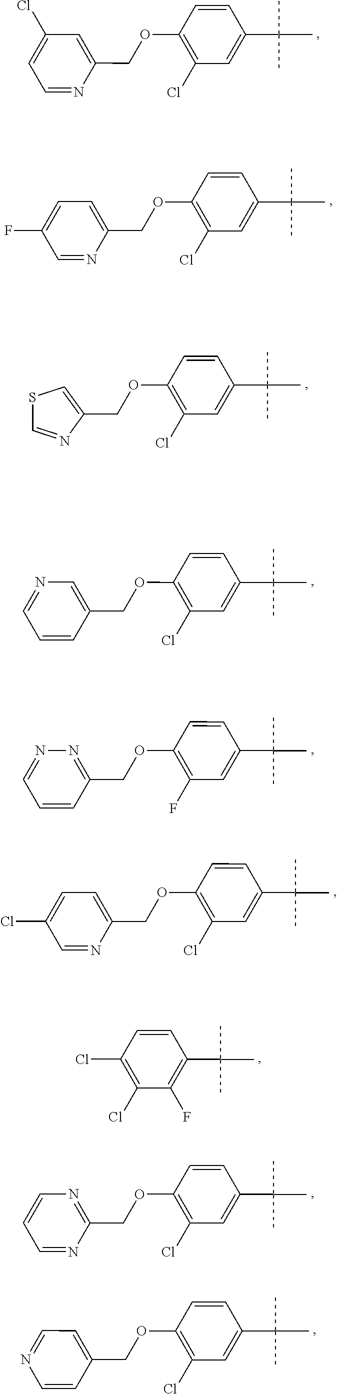

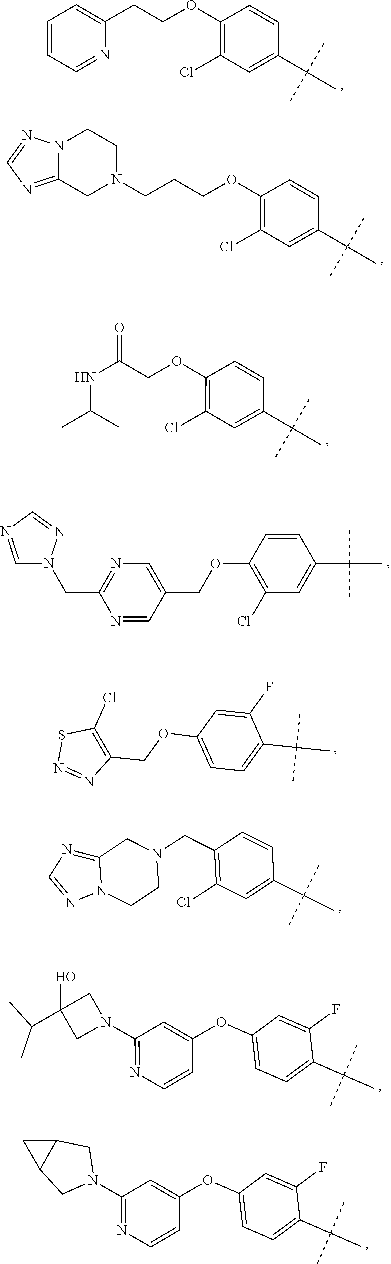

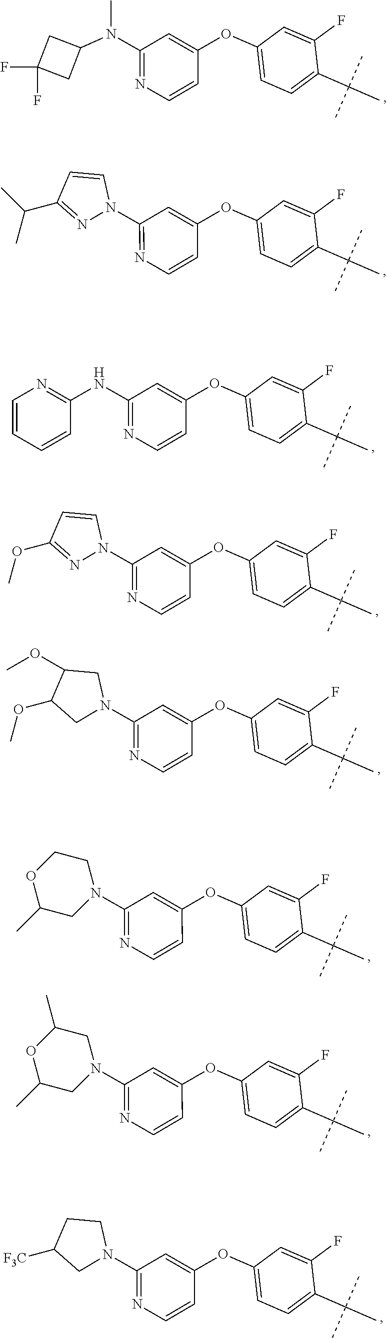

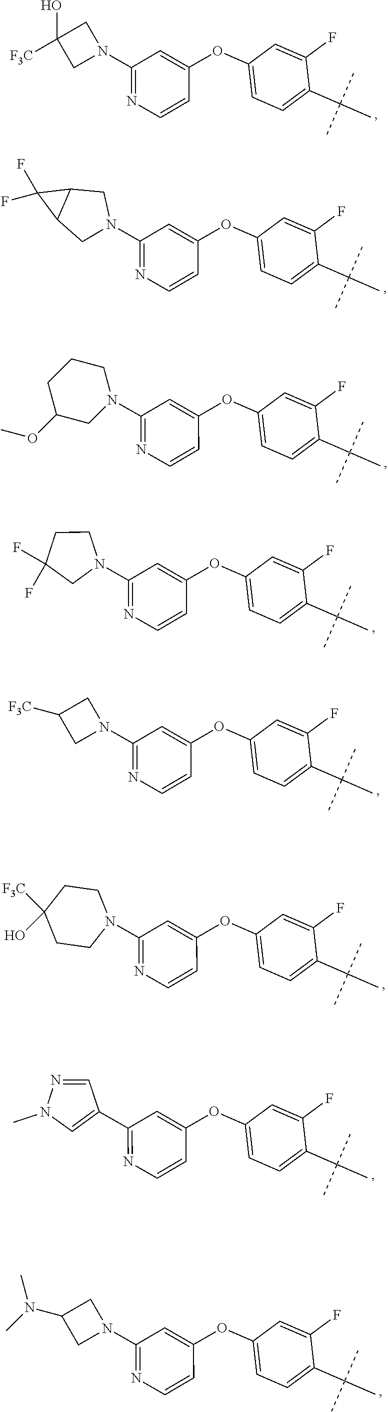

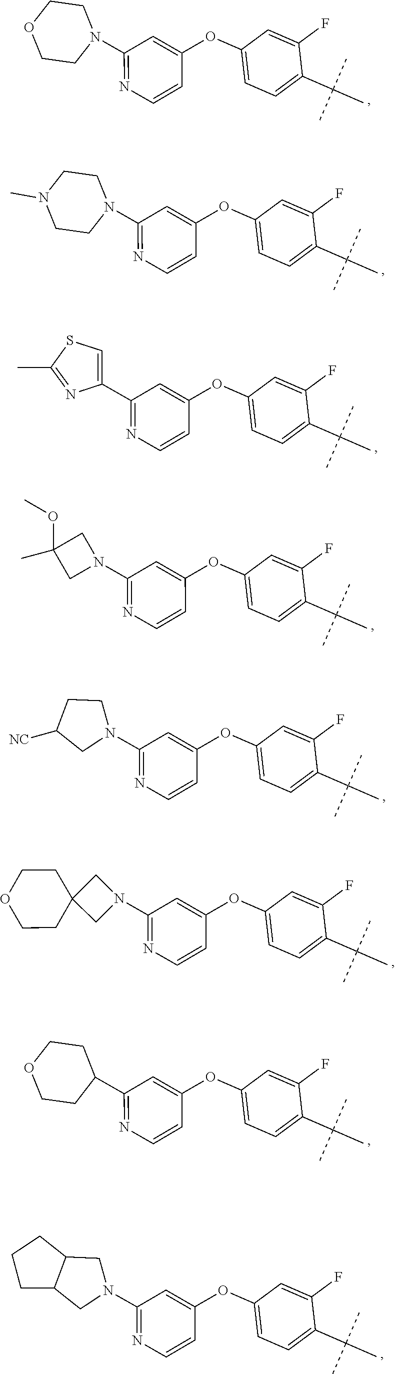

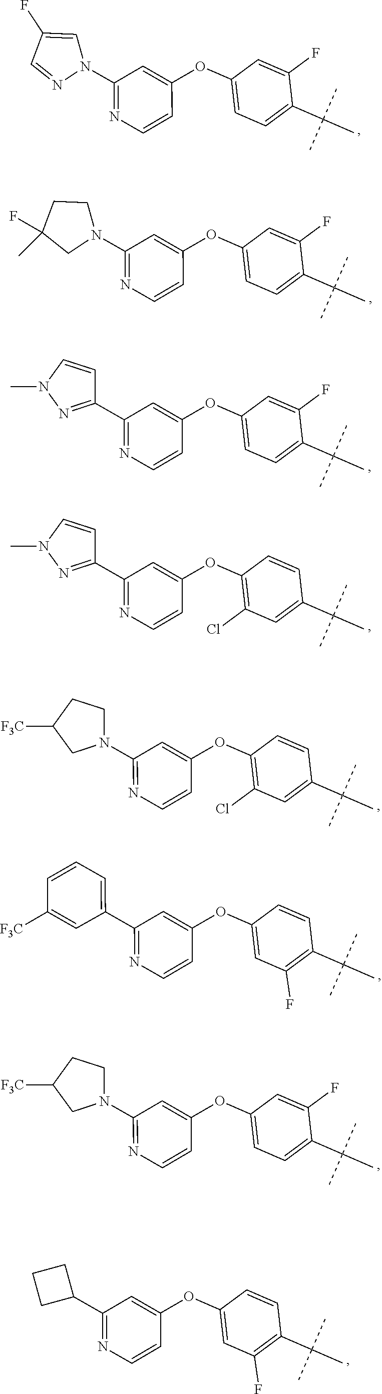

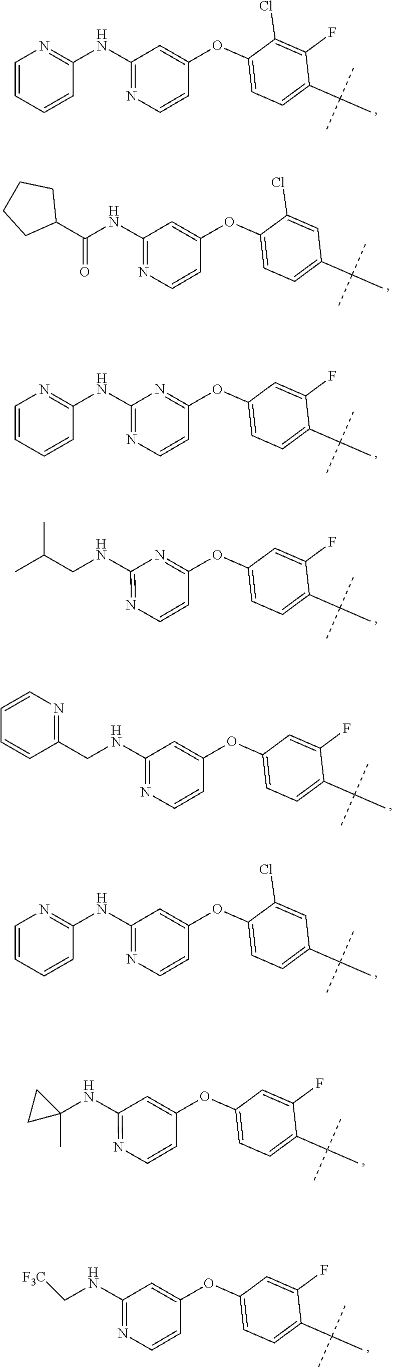

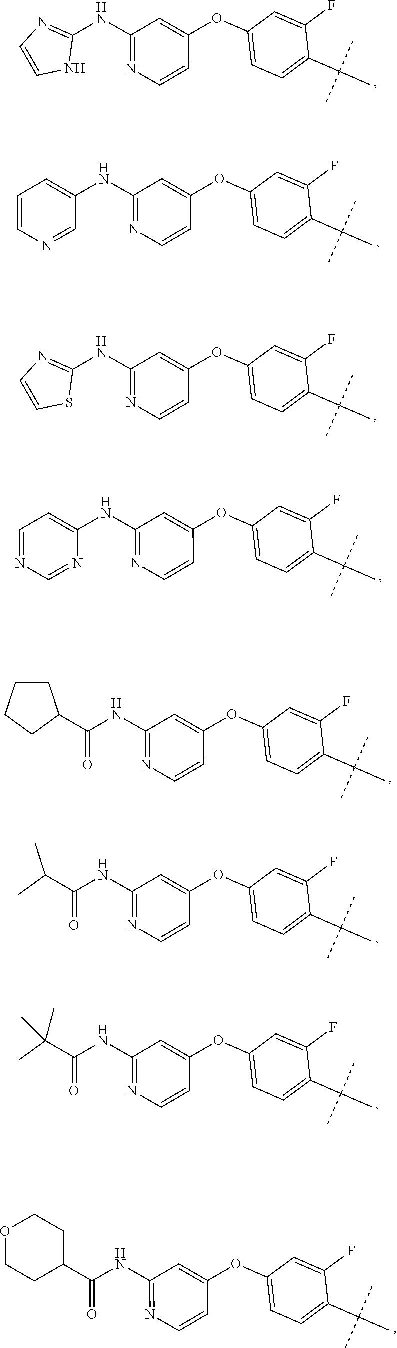

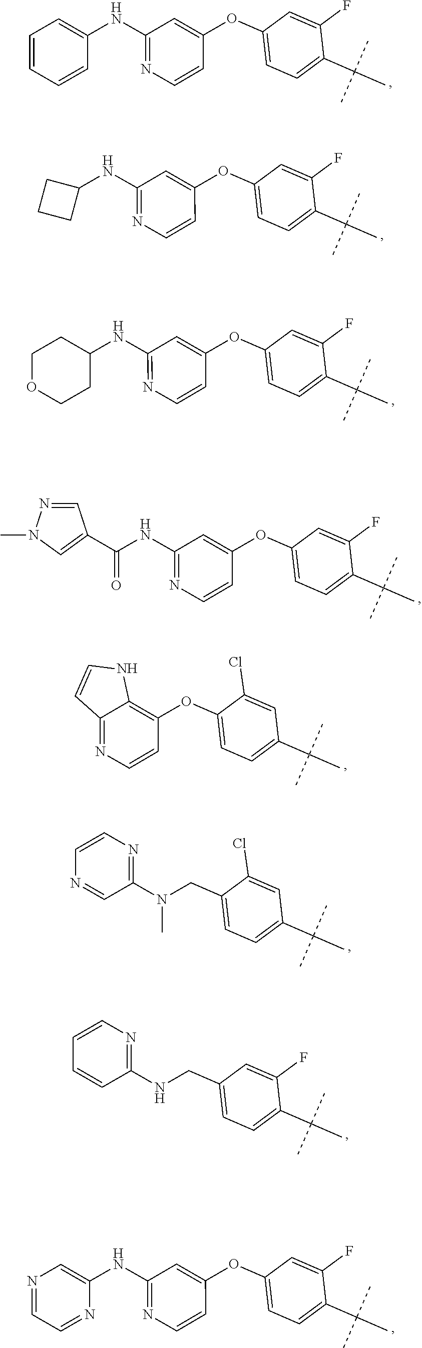

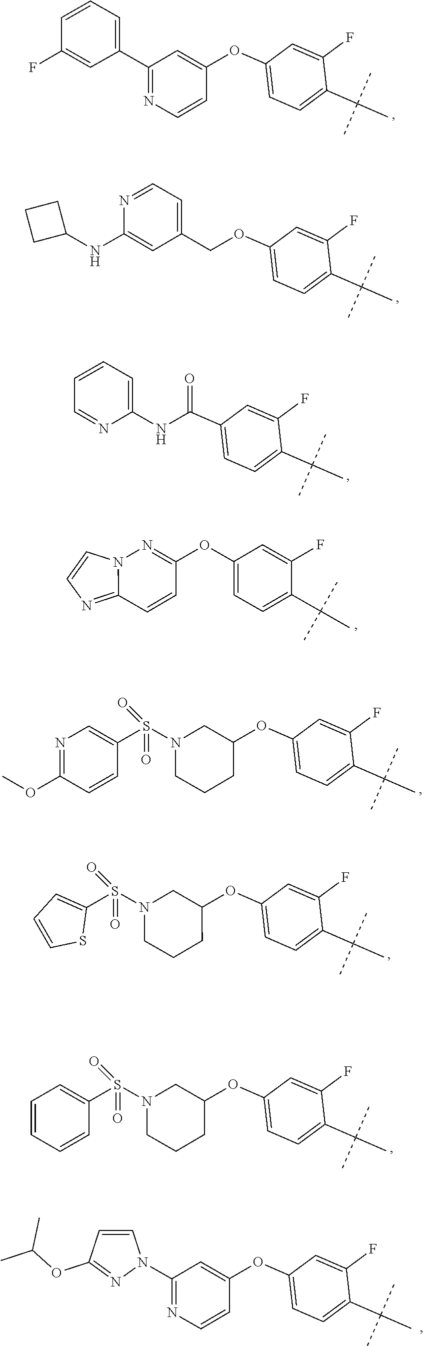

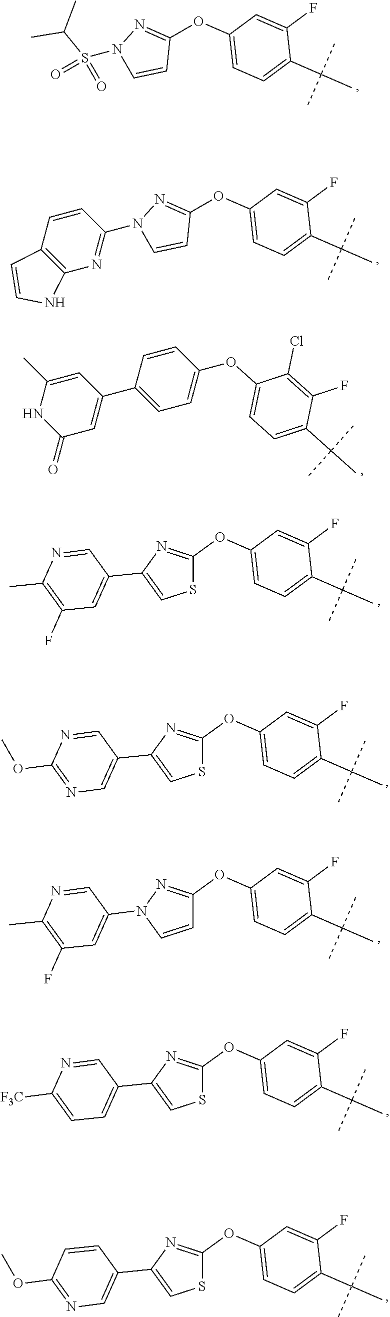

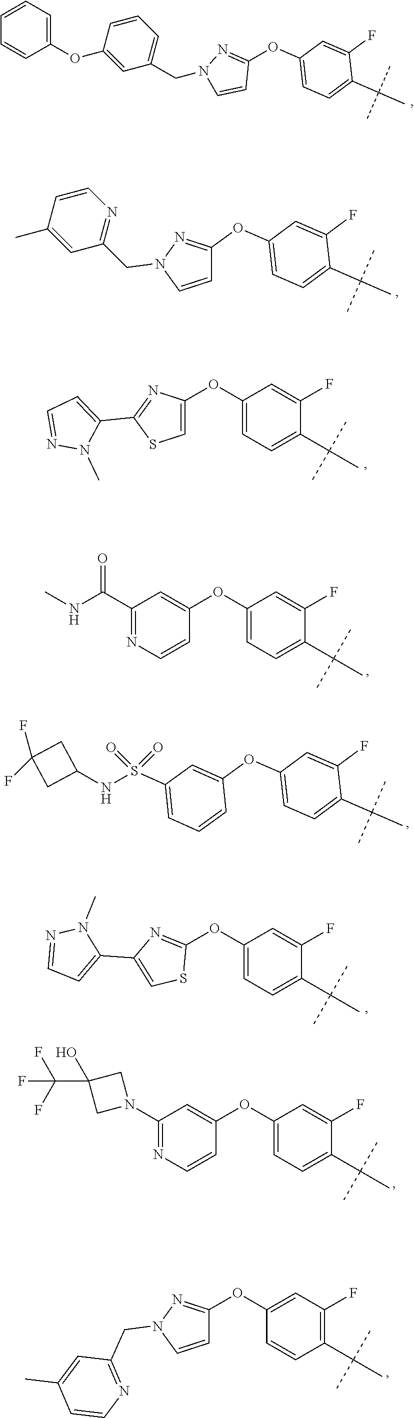

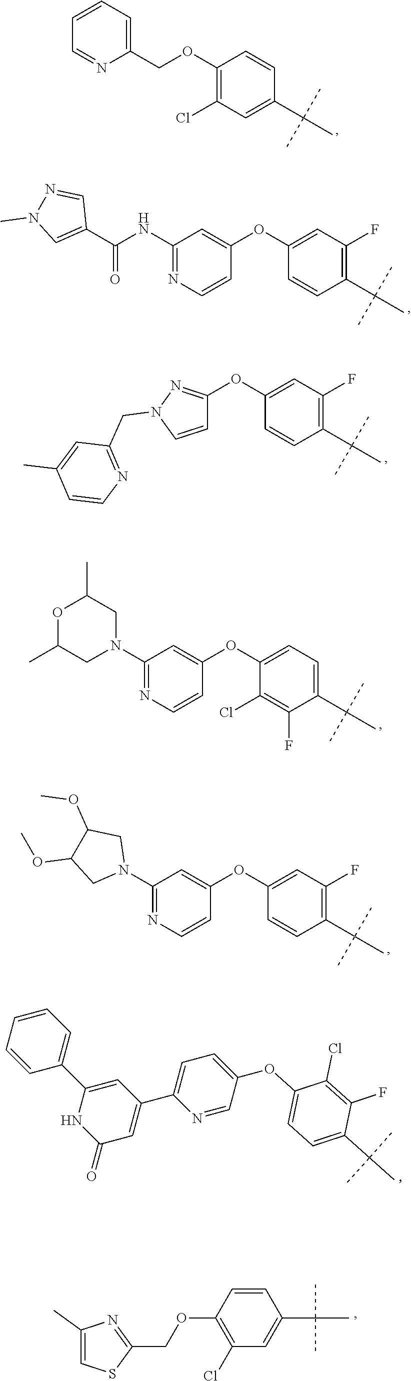

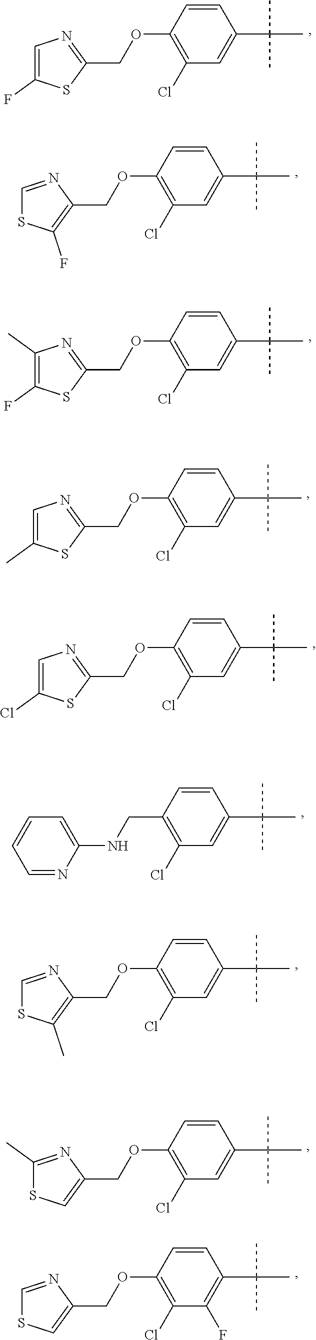

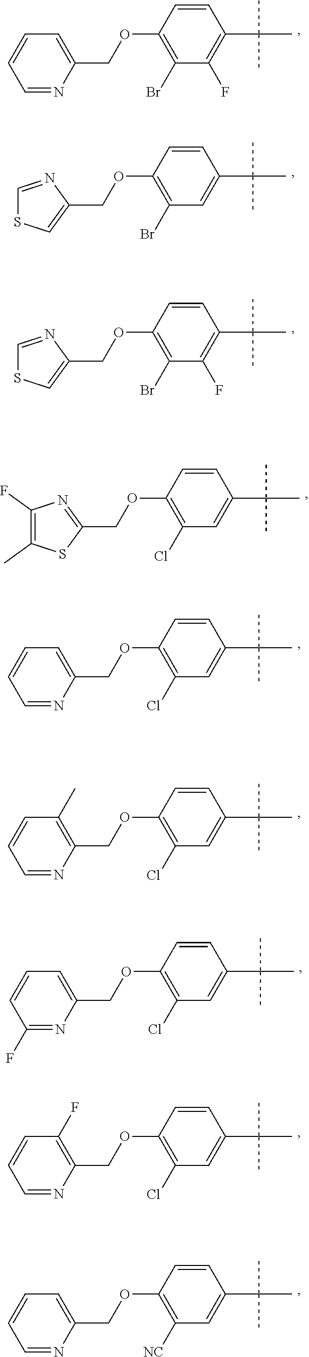

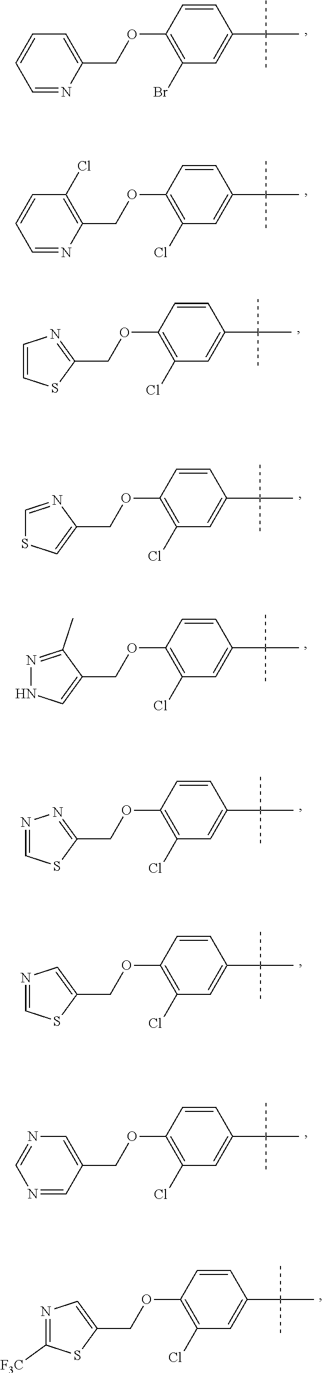

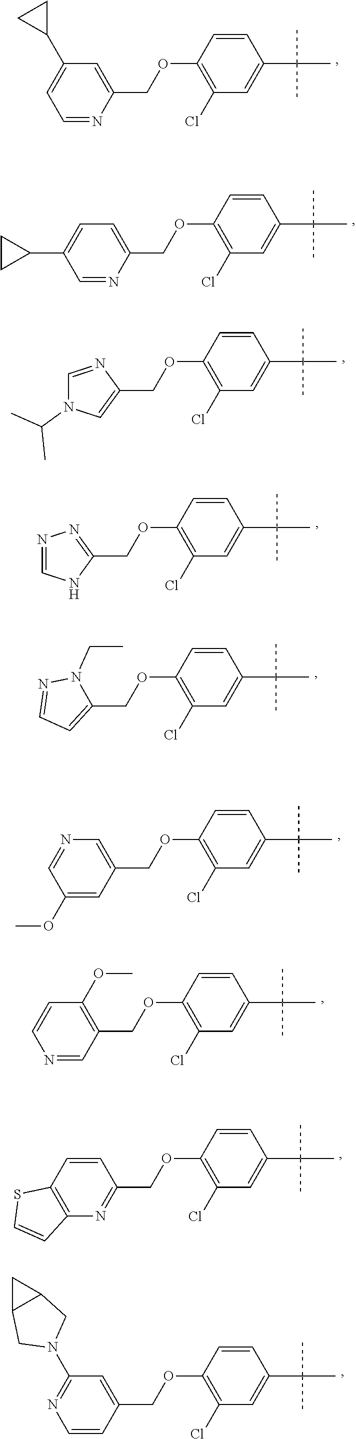

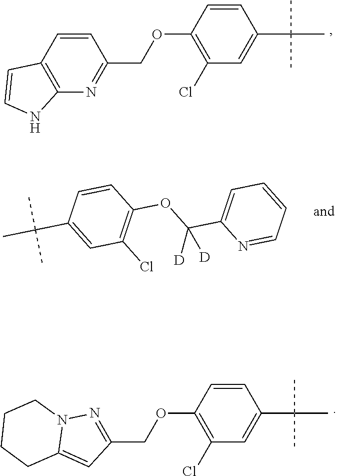

In some embodiments, R₂ is chosen from

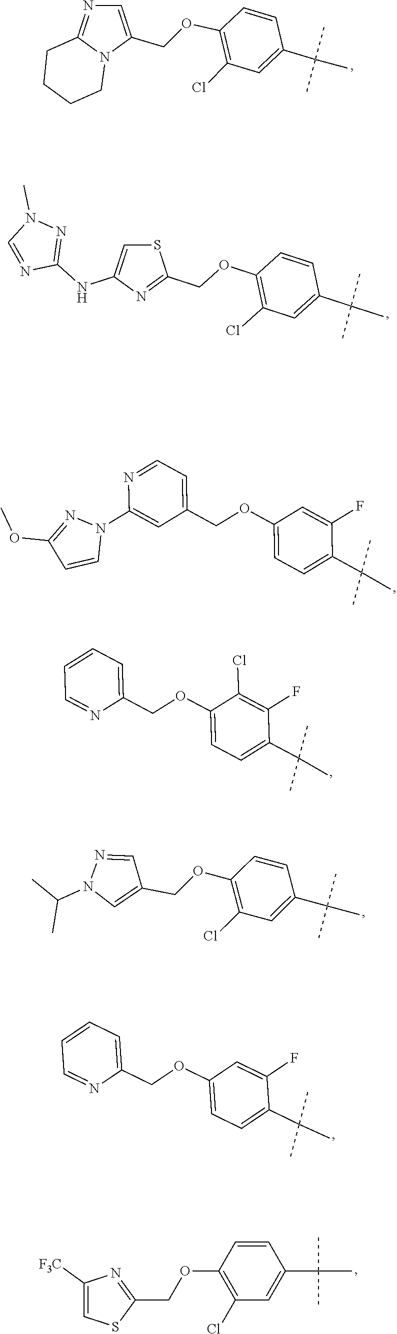

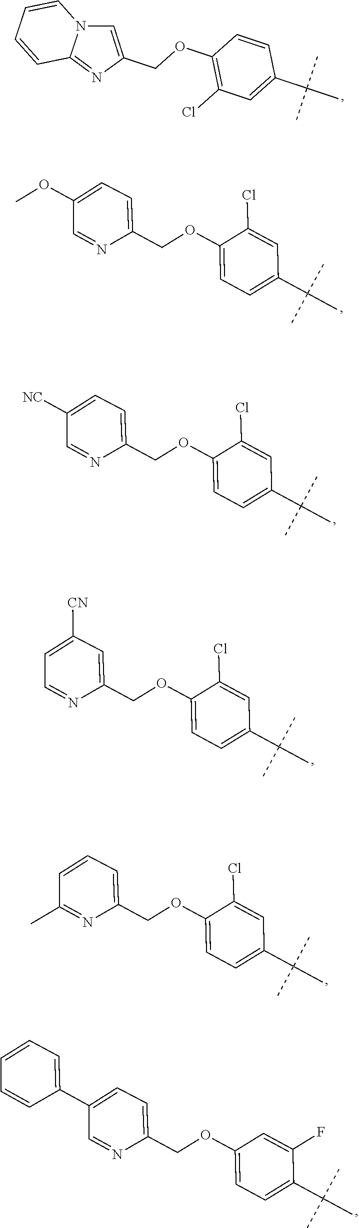

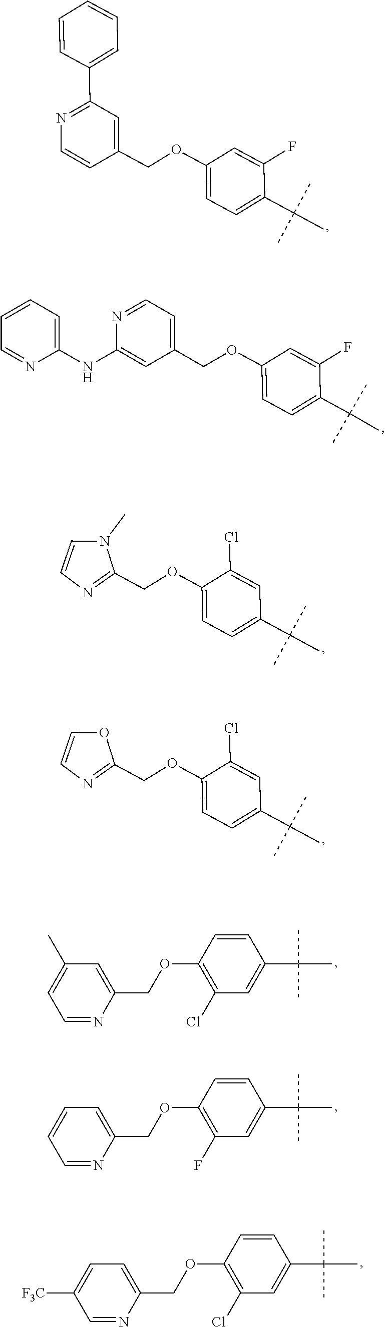

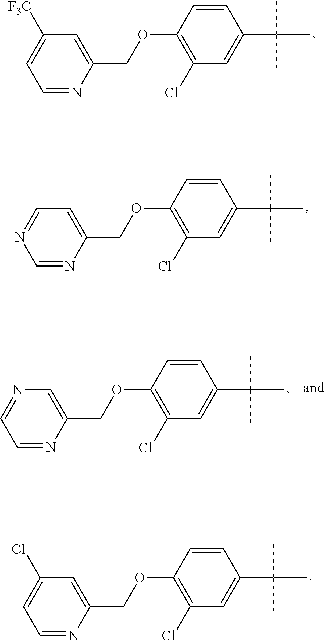

In some embodiments, R₂ is chosen from

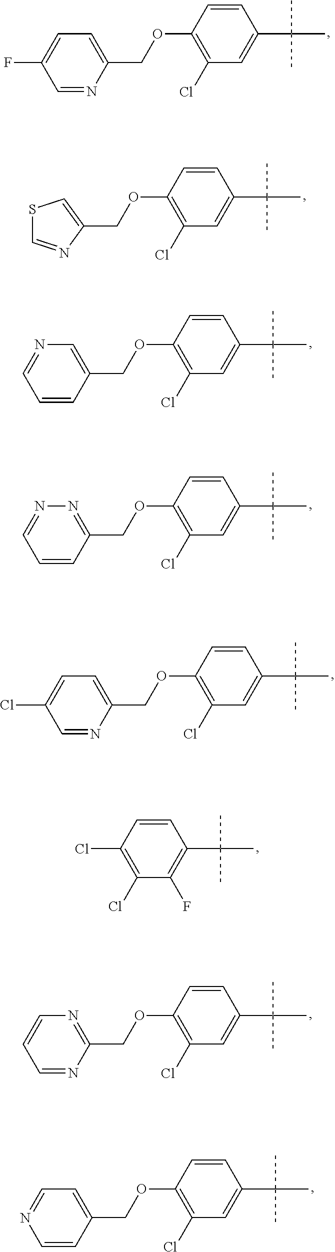

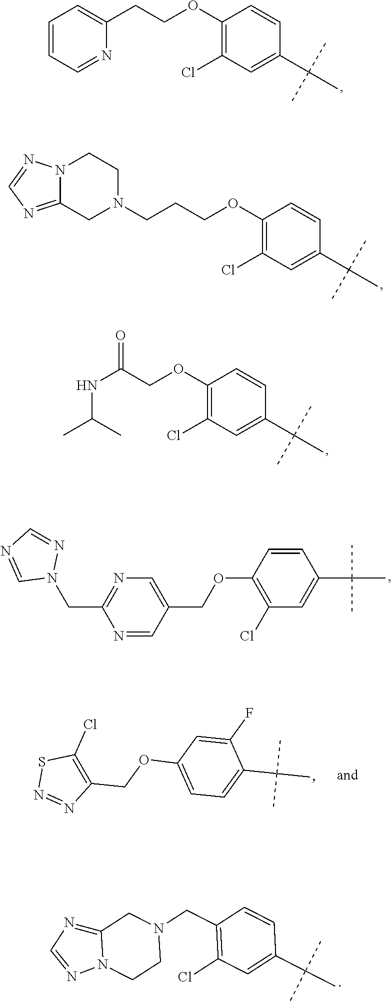

In some embodiments, R₂ is chosen from

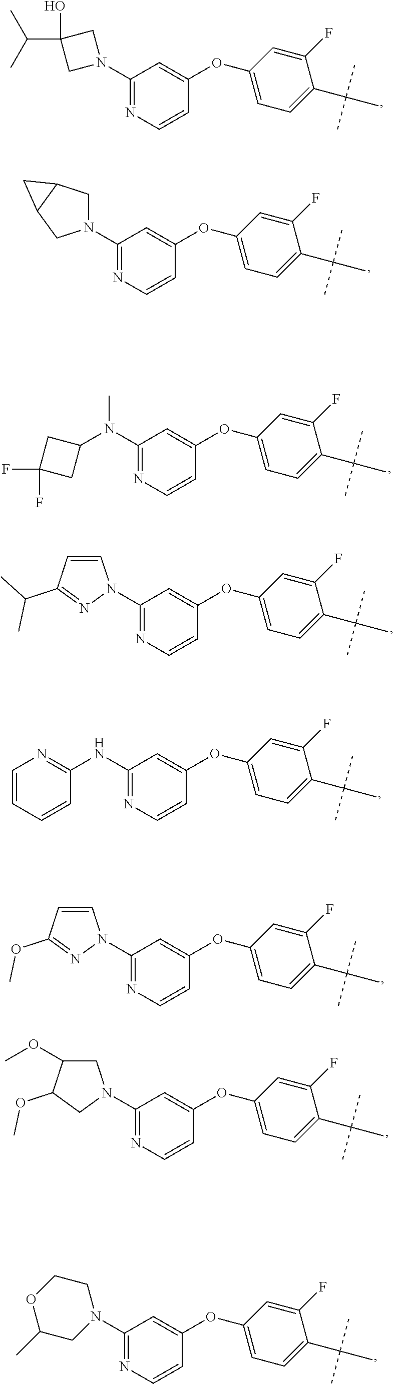

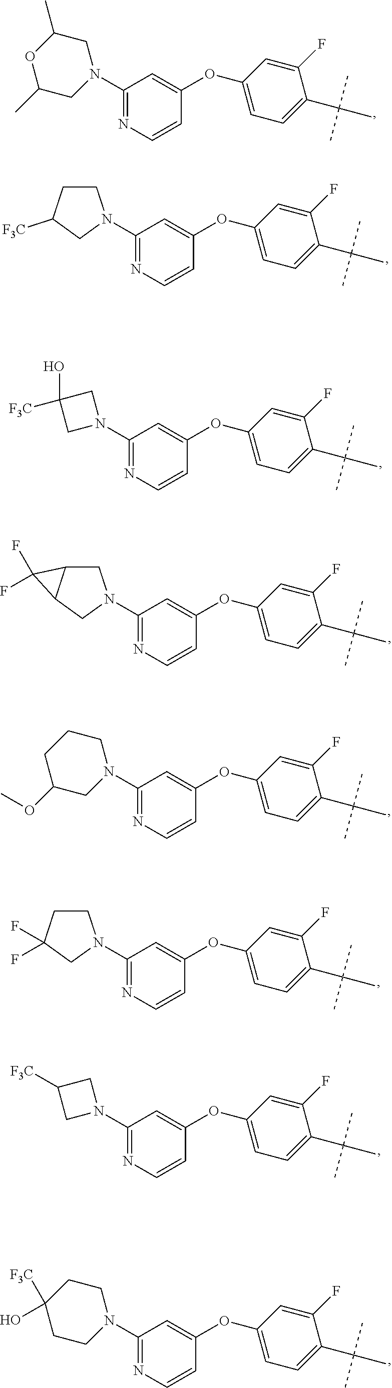

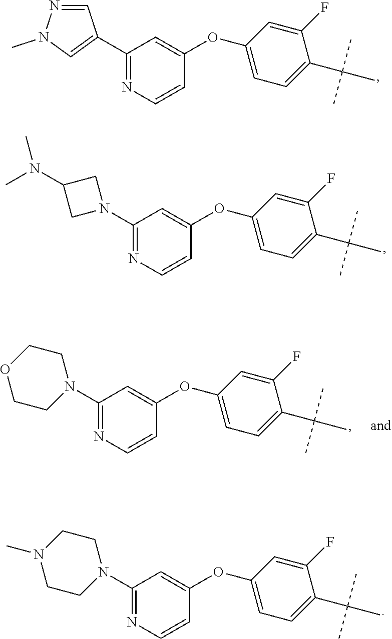

In some embodiments, R₂ is chosen from

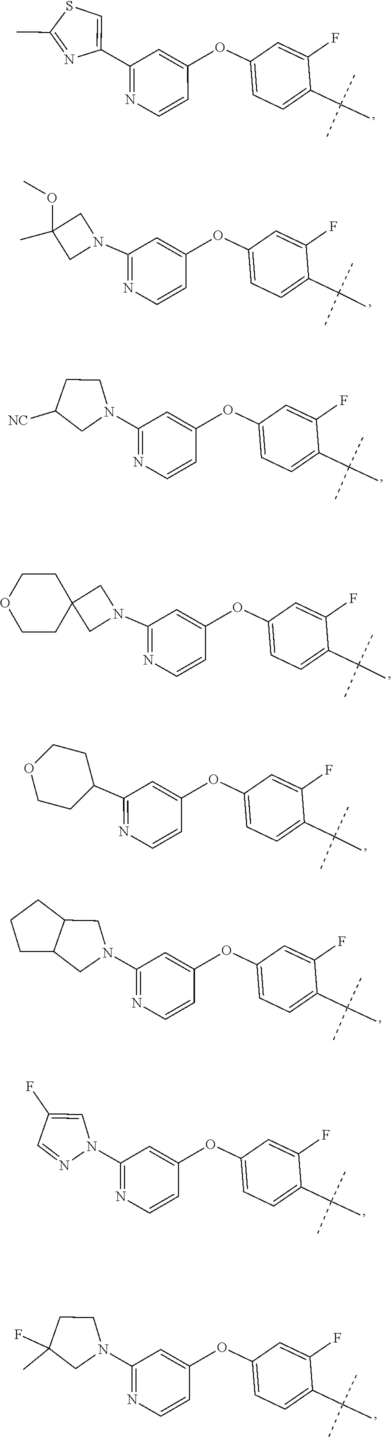

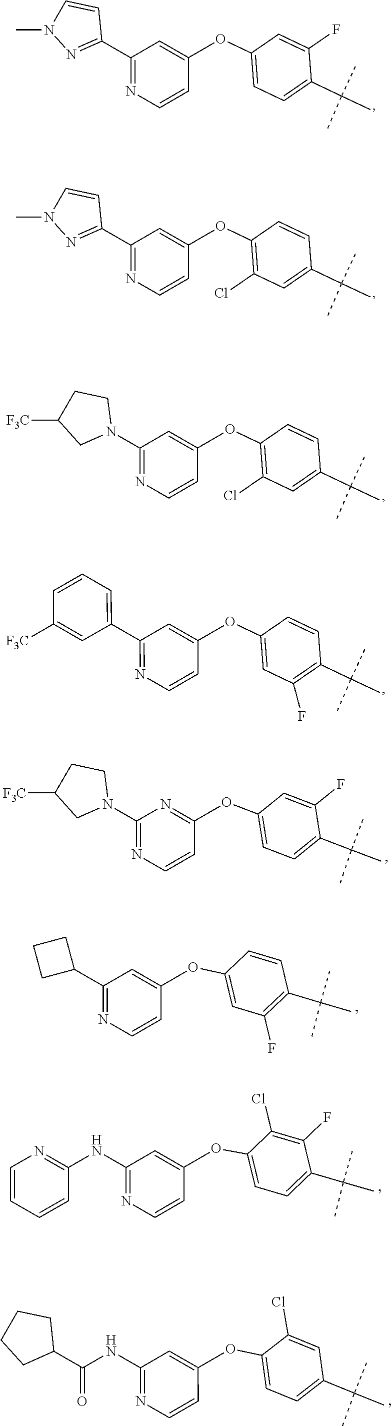

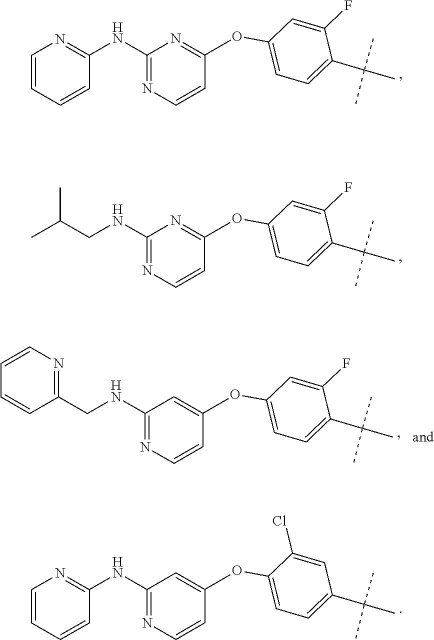

In some embodiments, R₂ is chosen from

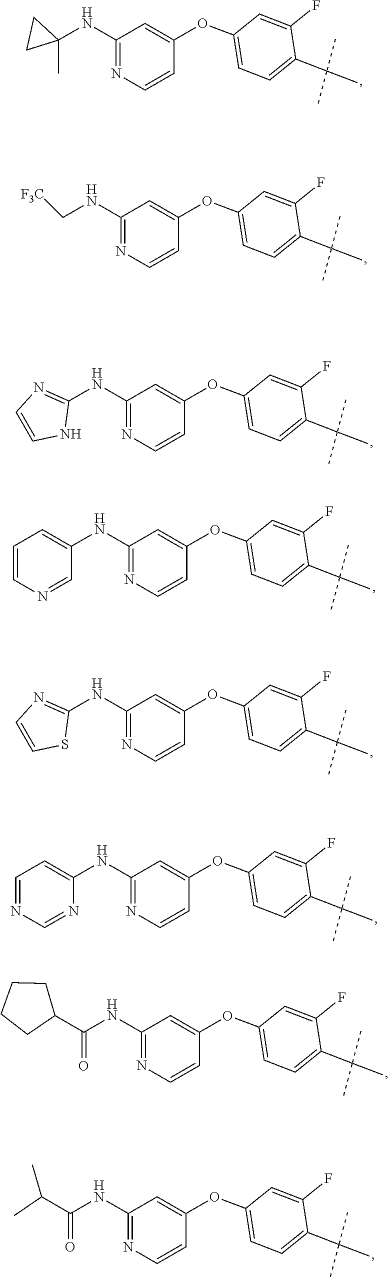

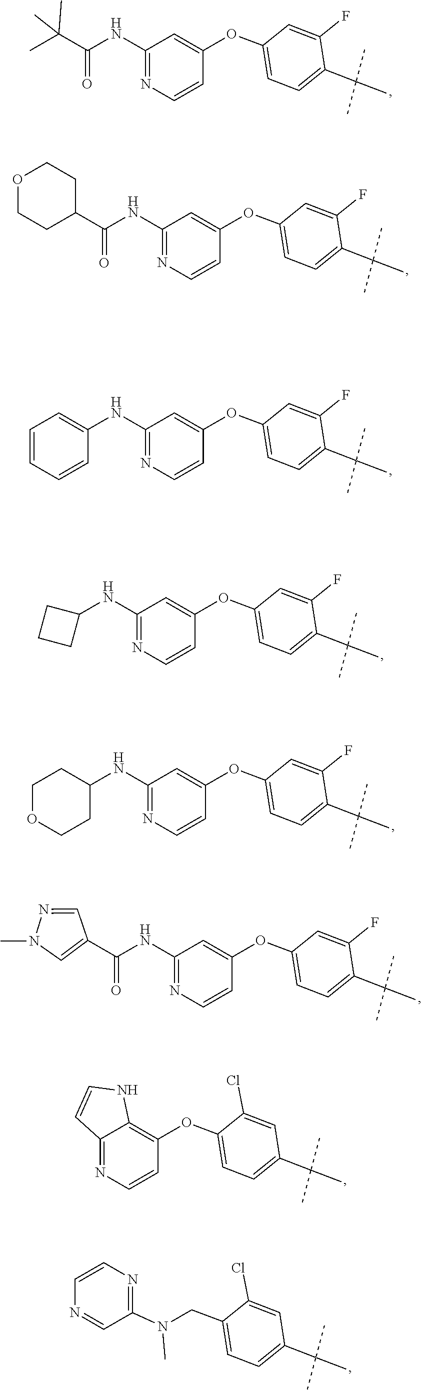

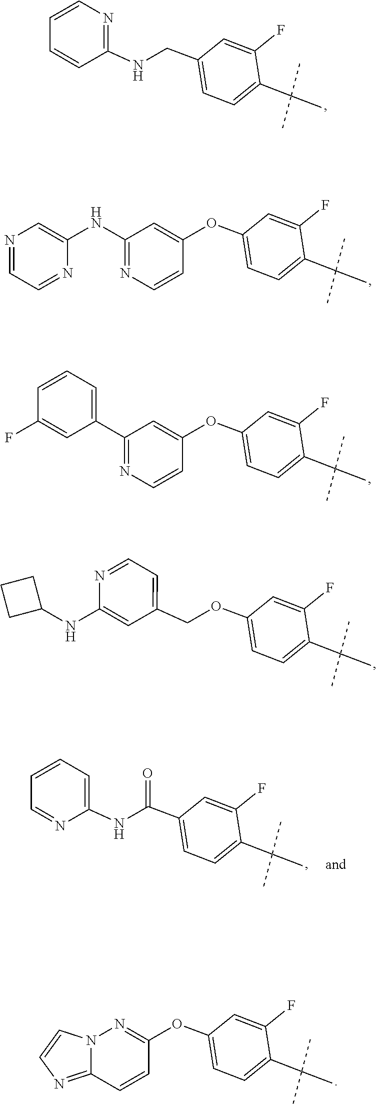

In some embodiments, R₂ is chosen from

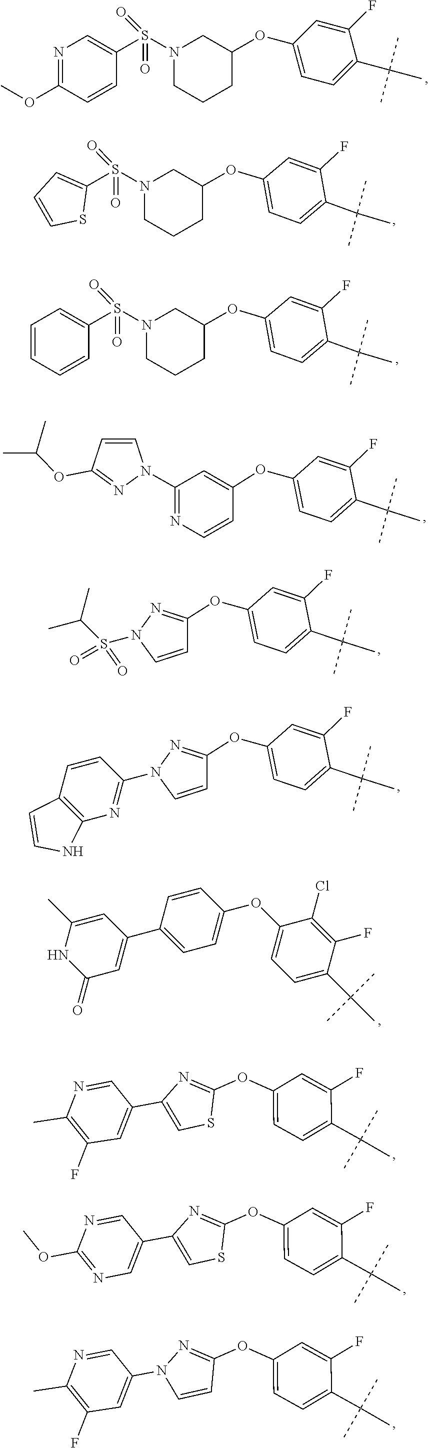

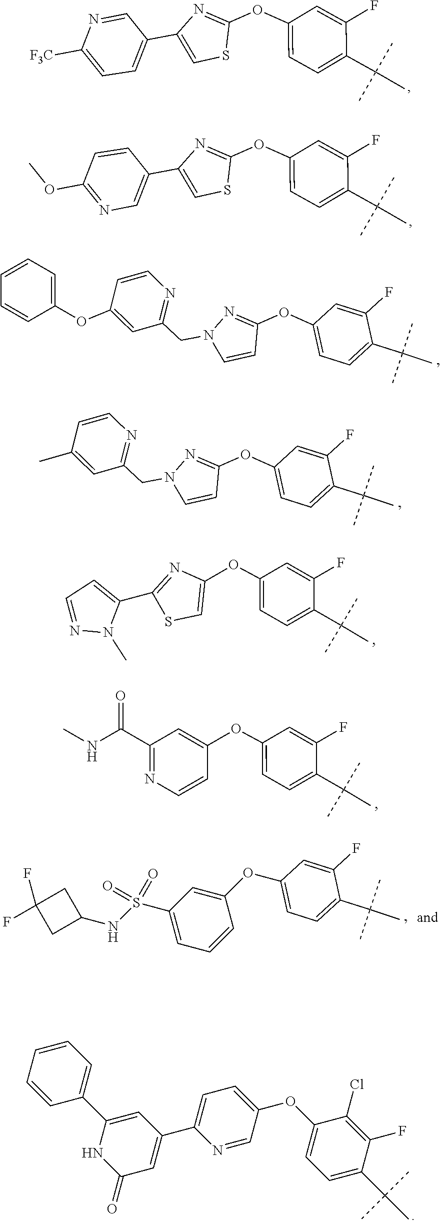

In some embodiments, R₂ is chosen from

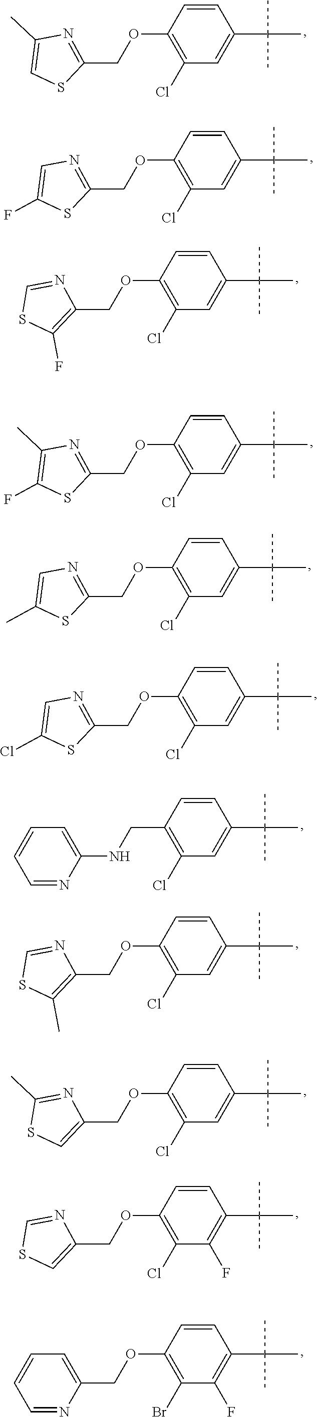

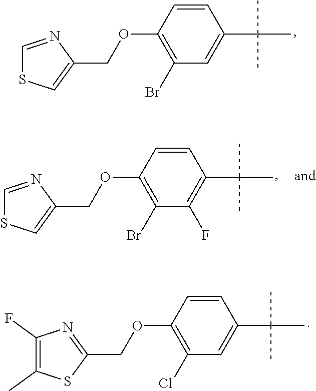

Disclosed are pharmaceutical compositions comprising a compound disclosed herein (e.g., Formula (1), Formula (2), Formula (3), Formula (4), Formula (5)) and at least one additional component chosen from pharmaceutically acceptable carriers, pharmaceutically acceptable vehicles, and pharmaceutically acceptable excipients.

In some embodiments, the compound disclosed herein is present in a therapeutically effective amount.

Disclosed are methods of treating cancer in a subject in need thereof, comprising administering to the subject an effective amount of a compound disclosed herein (e.g., Formula (1), Formula (2), Formula (3), Formula (4), Formula (5)) or of the pharmaceutical composition disclosed herein.

In some embodiments, the cancer is associated with an EGFR mutation.

In some embodiments, the cancer is chosen from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, and esophageal cancer.

In some embodiments, the cancer is lung cancer.

In some embodiments, the cancer is non-small cell lung cancer.

In some embodiments, the cancer is pancreatic cancer.

In some embodiments, the cancer is colon cancer.

In some embodiments, the cancer is breast cancer.

In some embodiments, the cancer is head and neck cancer.

In some embodiments, the cancer is sinonasal squamous cell carcinoma.

Disclosed are uses of the disclosed compounds or of the disclosed pharmaceutical compositions, in the preparation of a medicament.

In some embodiments, the method further comprises administering to a subject a compound of Formula (1), or a pharmaceutically acceptable salt thereof, or a pharmaceutical composition comprising a compound of Formula (1) or a pharmaceutically acceptable salt thereof in combination with another agent.

In some embodiments, the agent is chosen from gemcitabine, cisplatin, erlotinib, gefitinib, pemetrexed, bevacizumab, cetuximab, trastuzumab, pertuzumab, sorafenib, lapatinib, cobimetinib, selumetinib, and everolimus.

In some embodiments, the disclosed compounds or the disclosed pharmaceutical compositions and the additional agent are administered concomitantly.

In some embodiments, the disclosed compounds or the disclosed pharmaceutical compositions and the additional agent are administered sequentially.

### DETAILED DESCRIPTION OF THE DISCLOSURE

### Definitions

A dash (“-”) that is not between two letters or symbols is used to indicate a point of attachment for a substituent. For example, —CN is attached through the carbon atom.

When a range of values is listed, it is intended to encompass each value and sub-range within the range. For example, “C1-C6 alkyl” or “C1-6alkyl” is intended to encompass C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6 alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl.

The term “alkenyl” as used herein refers to an unsaturated straight or branched hydrocarbon having at least one carbon-carbon double bond, such as a straight or branched group of 2 to 8 carbon atoms, referred to herein as C2-8alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl, 2-butenyl, and 4-(2-methyl-3-butene)-pentenyl.

The term “alkyl” as used herein refers to a saturated straight or branched hydrocarbon, such as a straight or branched group of 1 to 8 carbon atoms, referred to herein as C1-8alkyl. Exemplary alkyl groups include, but are not limited to, methyl, ethyl, propyl, isopropyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-1-pentyl, 3 methyl-1-pentyl, 4-methyl-1-pentyl, 2-methyl-2-pentyl, 3-methyl-2-pentyl, 4 methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein refers to a divalent alkyl radical. Representative examples of C1-10 alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2 to 8 carbon atoms, referred to herein as C2-8alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “aryl” as used herein refers to a mono-, bi-, or other multi carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “C6-aryl.”

The term “cyano” as used herein refers to CN.

The term “cycloalkyl” as used herein refers to a saturated or unsaturated cyclic, bicyclic, or bridged bicyclic hydrocarbon group of 3-16 carbons, or 3-8 carbons, referred to herein as “C3-8cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

The term “heteroatoms,” as used herein, refers to nitrogen (N), oxygen (O), sulfur (S) or phosphorus (P) atoms, wherein the N, S and P can optionally be oxidized to various oxidation states.

The term “heteroaryl” as used herein refers to a mono-, bi-, or multi-cyclic, aromatic ring system containing one or more heteroatoms, for example 1 to 3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Exemplary heteroaryl groups include, but are not limited to, a monocyclic aromatic ring, wherein the ring comprises 2 to 5 carbon atoms and 1 to 3 heteroatoms, referred to herein as “C2-5heteroaryl.” Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups also include, but are not limited to, a bicyclic aromatic ring, wherein the ring comprises 5 to 14 carbon atoms and 1 to 3 heteroatoms, referred to herein as “C5-14heteroaryl.” Representative examples of heteroaryl include, but not limited to, indazolyl, indolyl, azaindolyl, indolinyl, benzotriazolyl, benzoxadiazolyl, imidazolyl, cinnolinyl, imidazopyridyl, pyrazolopyridyl, pyrrolopyridyl, quinolinyl, isoquinolinyl, quinazolinyl, quinazolinonyl, indolinonyl, isoindolinonyl, tetrahydronaphthyl, tetrahydroquinolinyl, tetrahydroisoquinolinyl, and the like.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

“Spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. A (C3-12)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The terms “composition” or “pharmaceutical composition,” as used herein, refers to a mixture of at least one compound, such as a compound Formula (1), or a tautomer, stereoisomer or a mixture of stereoisomers, or a pharmaceutically acceptable salt, or hydrate, or deuterated derivative thereof, with at least one and optionally more than one other pharmaceutically acceptable chemical components, such as carriers, stabilizers, diluents, dispersing agents, suspending agents, thickening agents, and/or excipients.

The terms “effective amount” or “therapeutically effective amount,” as used herein, refer to a sufficient amount of a compound described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “pharmaceutically acceptable,” as used herein, refers to a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “carrier,” as used herein, refers to chemical compounds or agents that facilitate the incorporation of a compound described herein into cells or tissues. The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329; Remington: The Science and Practice of Pharmacy, 21^{st} Ed. Pharmaceutical Press 2011; and subsequent versions thereof). Except insofar as any conventional carrier is incompatible with the active ingredient, its use in the therapeutic or pharmaceutical compositions is contemplated.

The term “pharmaceutically acceptable carrier” or “pharmaceutically acceptable vehicle” is used interchangeably and as is known to those skilled in the art, can be any and all solvents, dispersion media, Coatings, surfactants, antioxidants, preservatives (e.g., antimicrobial agents, antifungal agents), isotonic agents, absorption delaying agents, salts, preservatives, drug stabilizers, binders, excipients, disintegrants, lubricant, including sweeteners, flavors, dyes, and the like, and combinations thereof (e.g., Remington's Pharmaceutical Sciences, 18th Ed. Mack Printing Company, 1990, pp. 1289-1329). Except insofar as any conventional carrier is incompatible with the active ingredient, its use in therapeutic or pharmaceutical compositions is contemplated.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discemible by the patient. In yet another embodiment, “treat,” “treating,” or “treatment” refers to modulating the disease or disorder, either physically (e.g., through stabilization of a discemible symptom), physiologically, (e.g., through stabilization of a physical parameter), or both. In yet another embodiment, “treat,” “treating,” or “treatment” refers to preventing or delaying the onset or development or progression of the disease or disorder.

As used herein, “cancer” refers to diseases, disorders, and conditions that involve abnormal cell growth with the potential to invade or spread to other parts of the body. Exemplary cancers include, but are not limited to, breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, a subject is “in need of” a treatment if such subject would benefit biologically, medically or in quality of life from such treatment.

As used herein, “exon 20 insertion mutation” refers to a mutation in which one or more amino acids (preferably 1 to 7, more preferably 1 to 4) are inserted in the exon 20 region of EGFR, and includes, but is not limited to, a mutation in which amino acid sequence SVD (serine, valine, and aspartic acid in this order from the N-terminus) is inserted between the 770th aspartic acid and 771st asparagine in the exon 20 region (D770_N771insSVD); a mutation in which amino acid sequence ASV (alanine, serine, and valine in this order from the N-terminus) is inserted between the 769th alanine and 770th aspartic acid in the exon region (A769_D770insASV); and a mutation in which amino acid sequence YVMA (tyrosine, valine, methionine, and alanine in this order from the N-terminus) is inserted between the 775th alanine and 776th glycine in the exon region (A775_G776ins YVMA).

The term “administration” or “administering” of the subject compound means providing a compound of the invention, a pharmaceutically acceptable salt, a pharmaceutically acceptable solvate, or solvate thereof to a subject in need of treatment.

The term “combination” as used herein means a product that results from the mixing or combining of more than one active ingredient and includes both fixed and non-fixed combinations of the active ingredients. The term “fixed combination” means that the active ingredients, by way of example, a compound of Formula (1), or a pharmaceutically acceptable salt thereof, and an additional anti-cancer agent, are both administered to a patient simultaneously in the form of a single entity or dosage. The term “non-fixed combination” means that the active ingredients, by way of example, a compound of Formula (1) or a pharmaceutically acceptable salt thereof, and an additional anti-cancer agent, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as 2H, 3H, 11C, 13C, 14C, 15N, 18F, 31P, 32P, 35S, 36Cl and 125I respectively. The invention includes various isotopically labeled compounds as defined herein, for example those into which radioactive isotopes, such as 3H, 13C, and 14C, are present. Such isotopically labelled compounds are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. Isotopically-labeled compounds of the invention can generally be prepared by conventional techniques known to those skilled in the art or by processes analogous to those described in the accompanying Examples and Processes using an appropriate isotopically-labeled reagents in place of the non-labeled reagent previously employed.

Compounds

Disclosed are compounds of Formula (1) or a tautomer, stereoisomer or a mixture of stereoisomers, or a pharmaceutically acceptable salt, or hydrate, or deuterated derivative thereof:

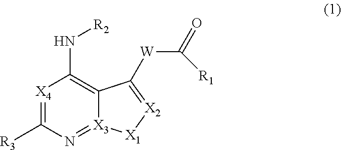

wherein:
X₁ is chosen from NH, N—CH₃, S, and CH;
X₂ is chosen from CH, N, C—CH₃, and N-methylpiperazine-4-phenyl;
X₃ is chosen from C and N;
X₄ is chosen from N, C—CN, and C—F;
R₃ is chosen from H and C₁-C₅ alkyl;
R₁ is chosen from

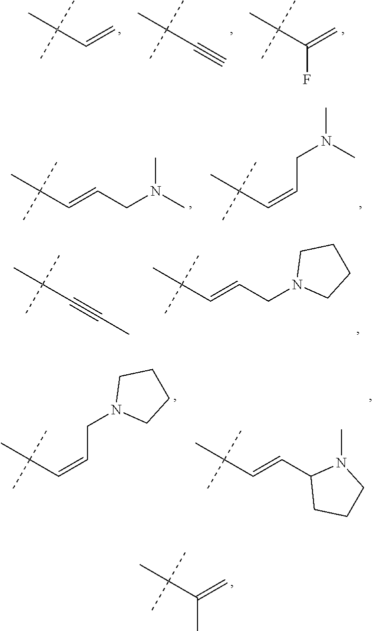

and C₁-C₁₀ alkyl;
W is chosen from alkylamino group, azetidine, methylazetidine, azetidine-3-amine, piperidine, tetrahydropyridine, pyrrolidine, 3-aminopyrrolidine, cyclobutylamine, cyclopentylamine, azepane, bicyclic amine, bridge cyclic amine, a spirocyclic amine, and an aryl amine, each of which is optionally substituted with halogen, hydroxyl, C₁-C₅ alkyl, and C₁-C₅ alkoxy; and
R₂ is chosen from

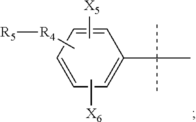

wherein:
X₅ and X₆ are each independently a halogen or a cyano group,
R₄ is chosen from a C₁-C₃ alkyl, a C₁-C₃ alkoxy,

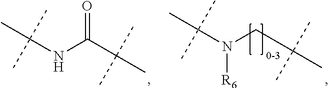

and O, and
R5 is chosen from optionally substituted aryls and optionally substituted heteroaryls;
wherein R6 is H or CH3.

In some embodiments, the compound of Formula (1) may be a compound of Formula (2):

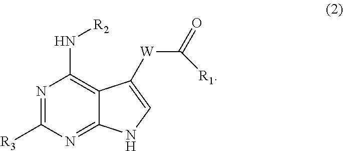

In some embodiments, the compound of Formula (1) may be a compound of Formula (3):

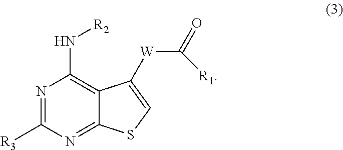

In some embodiments, the compound of Formula (1) may be a compound of Formula (4):

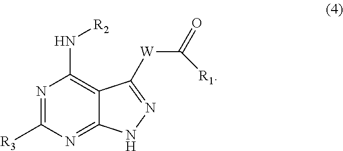

In some embodiments, the compound of Formula (1) may be a compound of Formula (5):

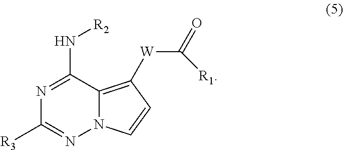

In some embodiments, W is chosen from

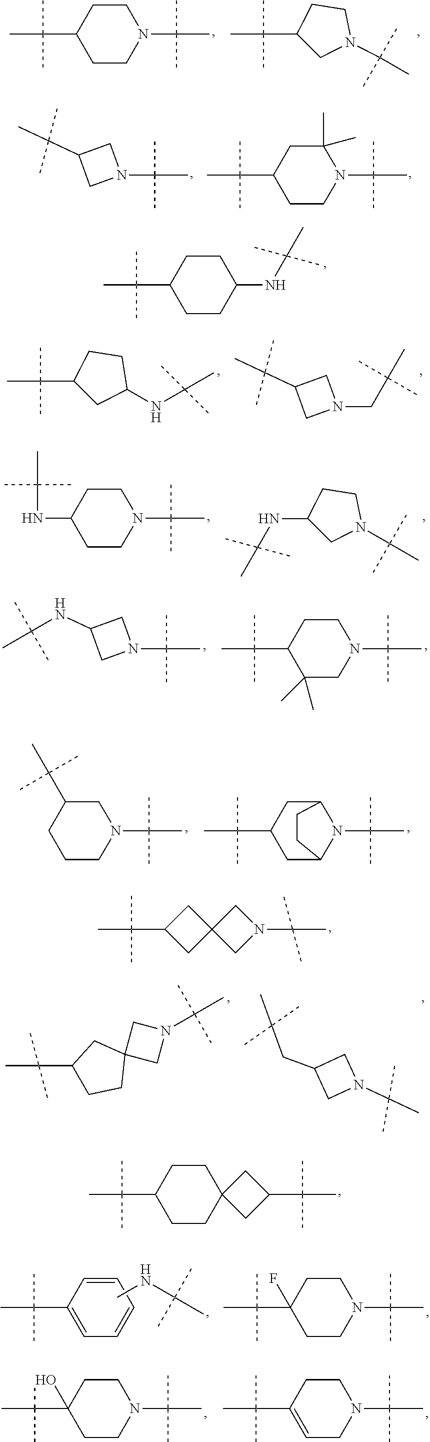

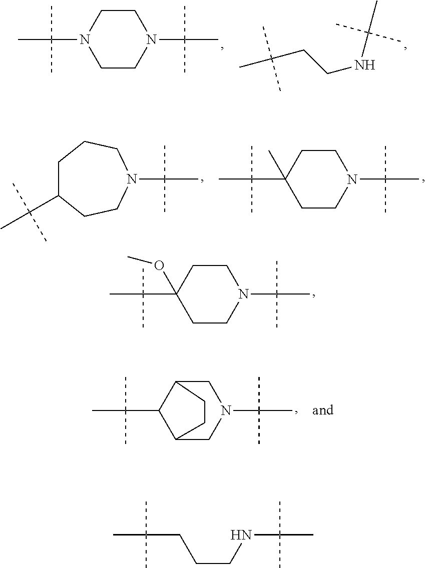

In some embodiments, R₂ is chosen from

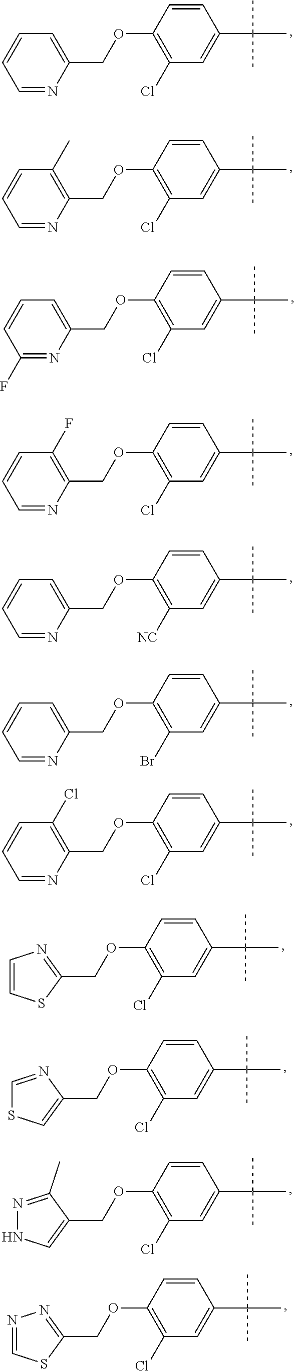

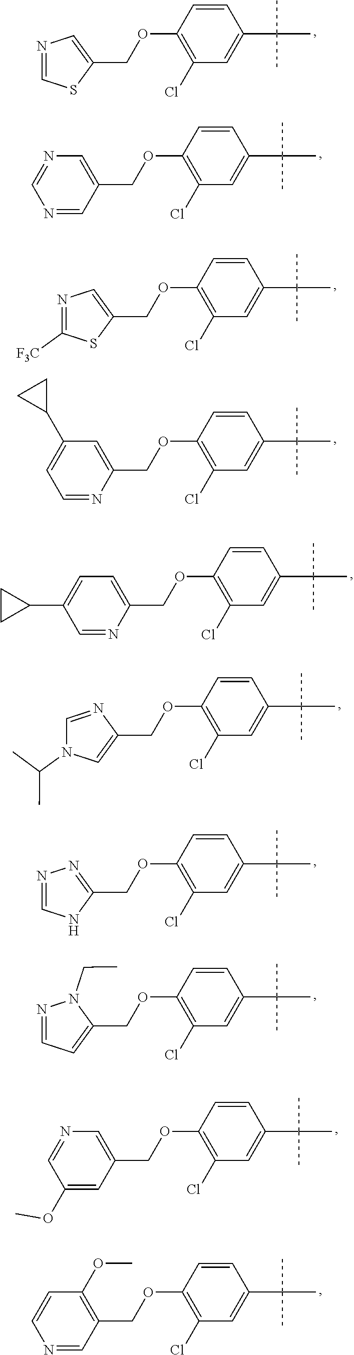

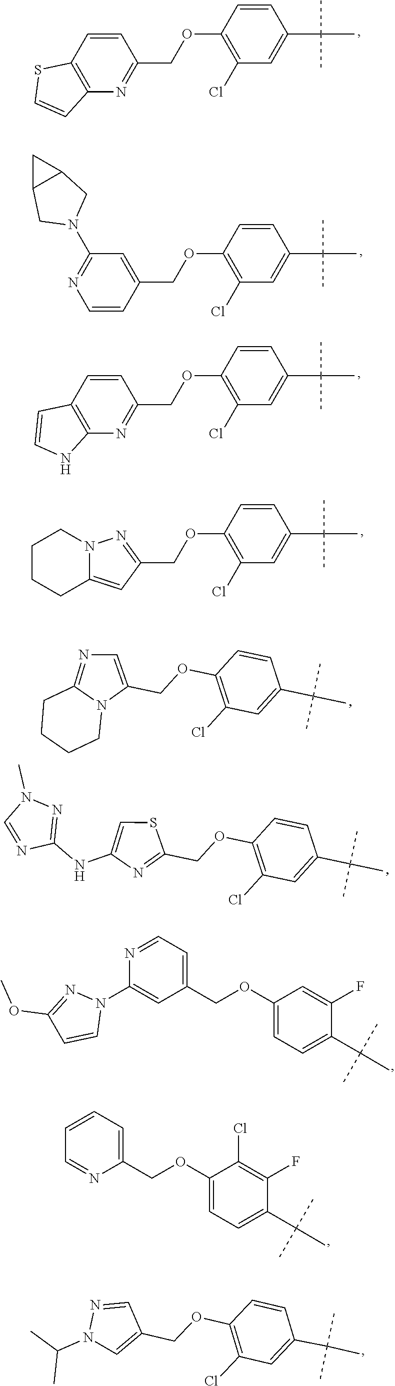

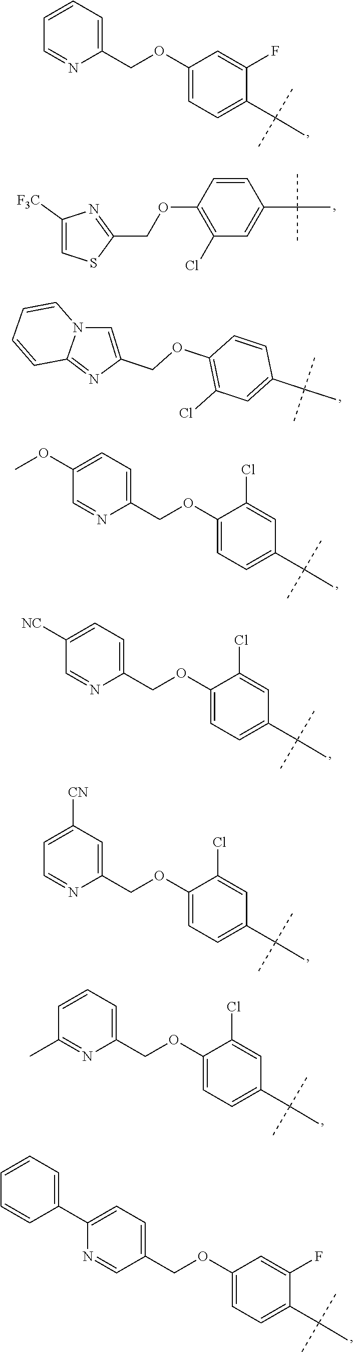

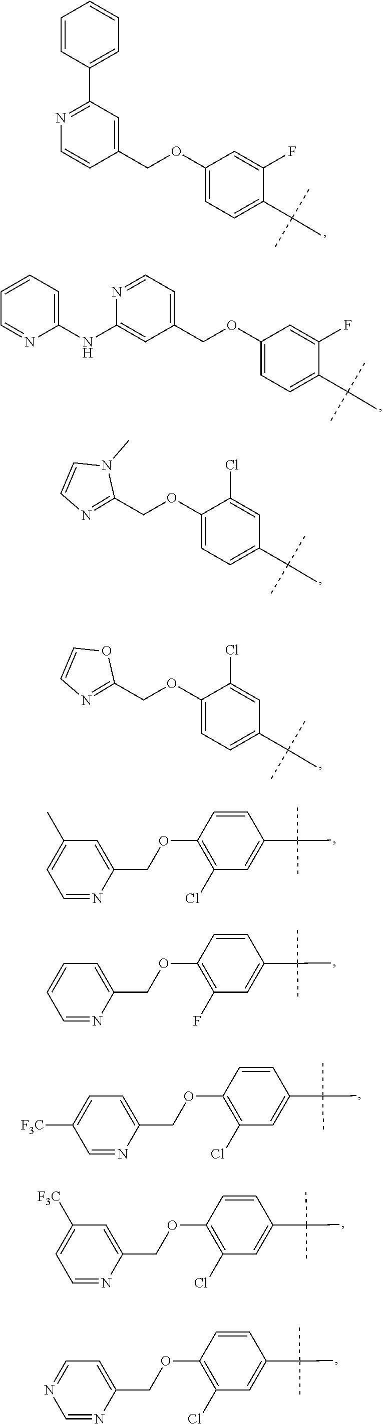

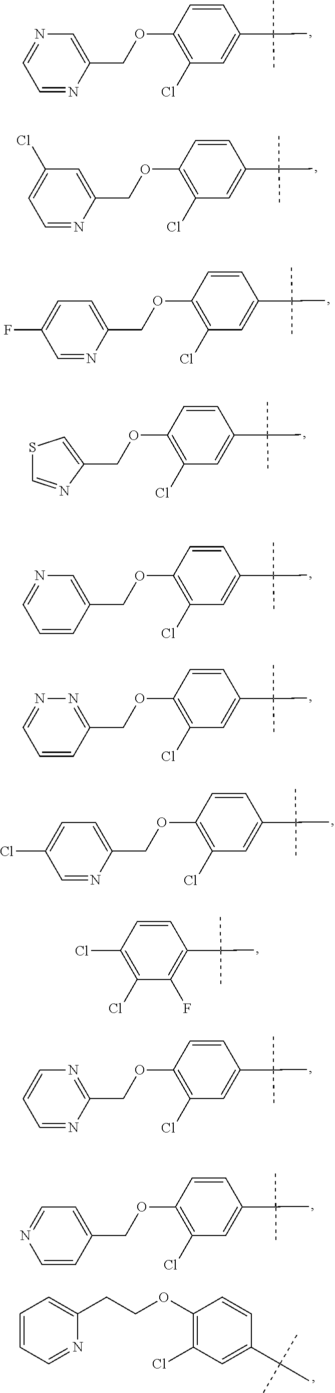

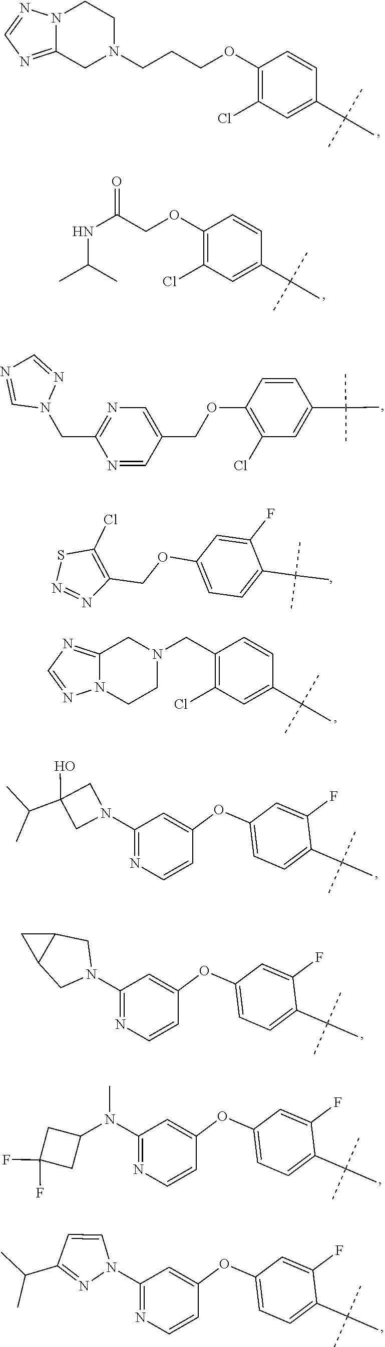

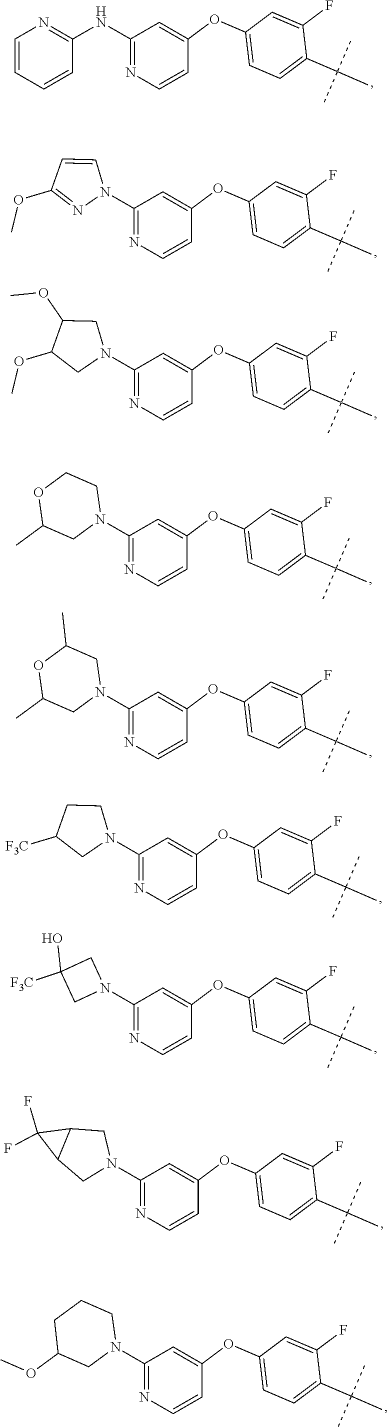

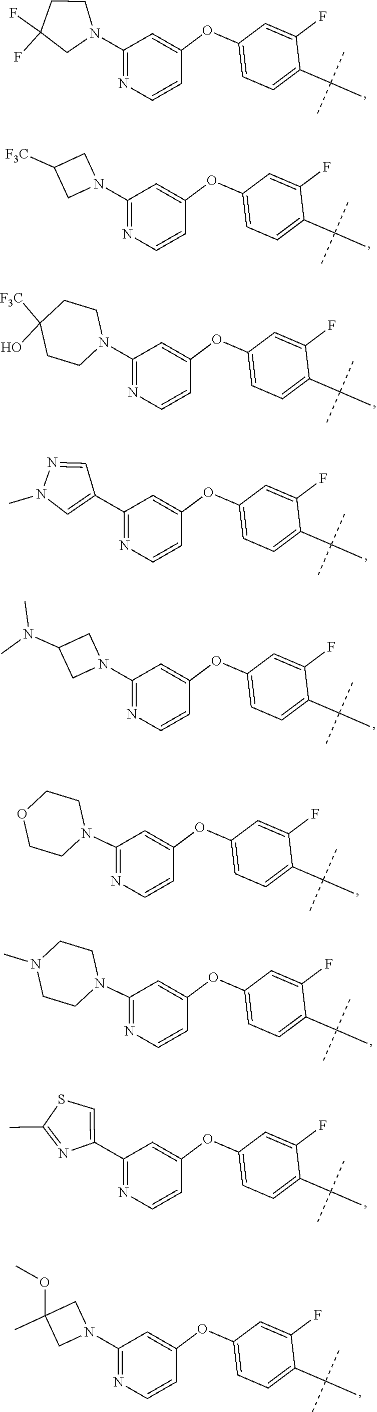

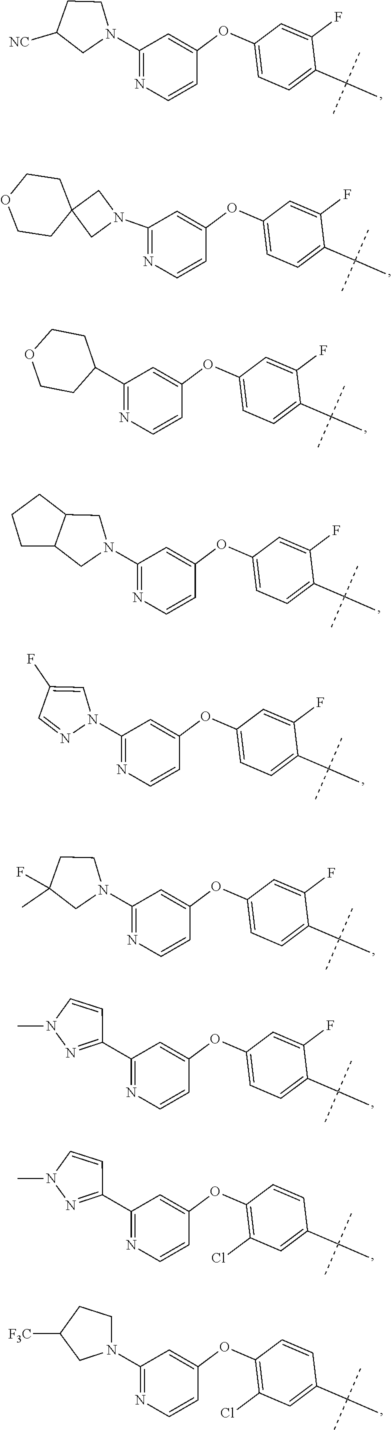

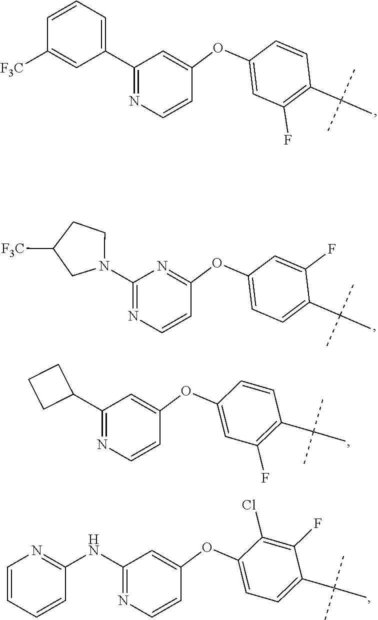

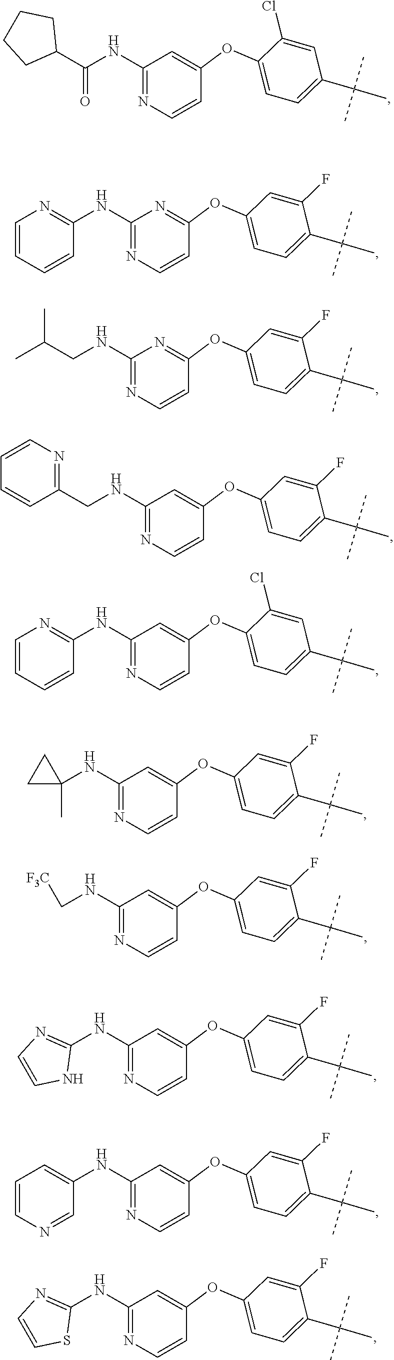

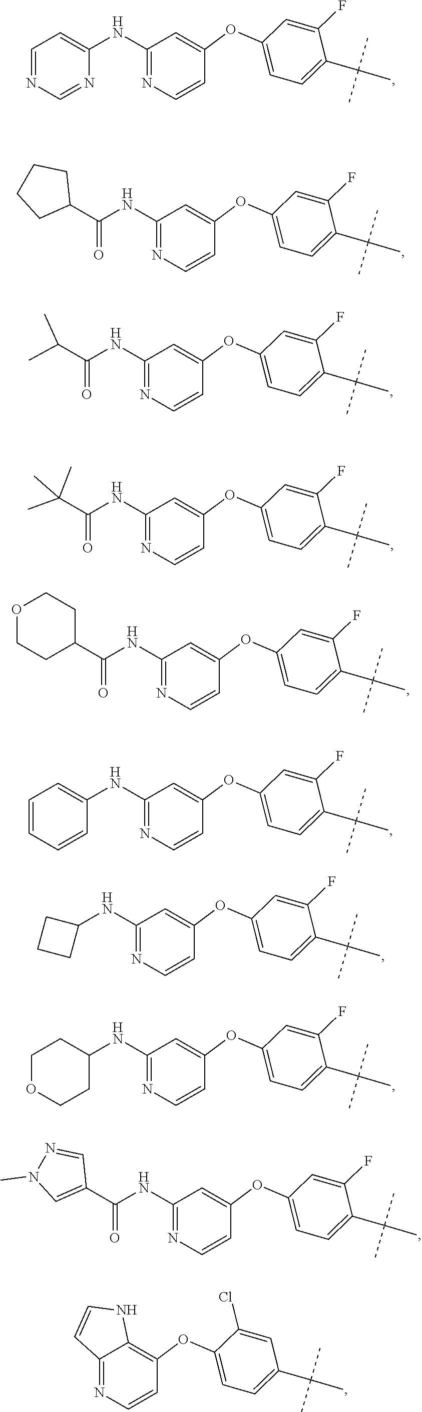

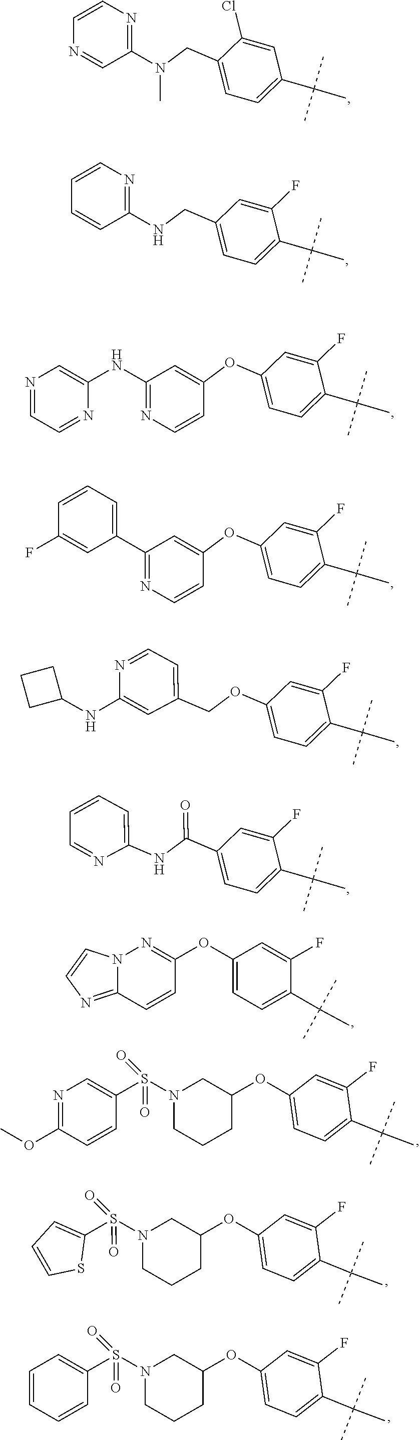

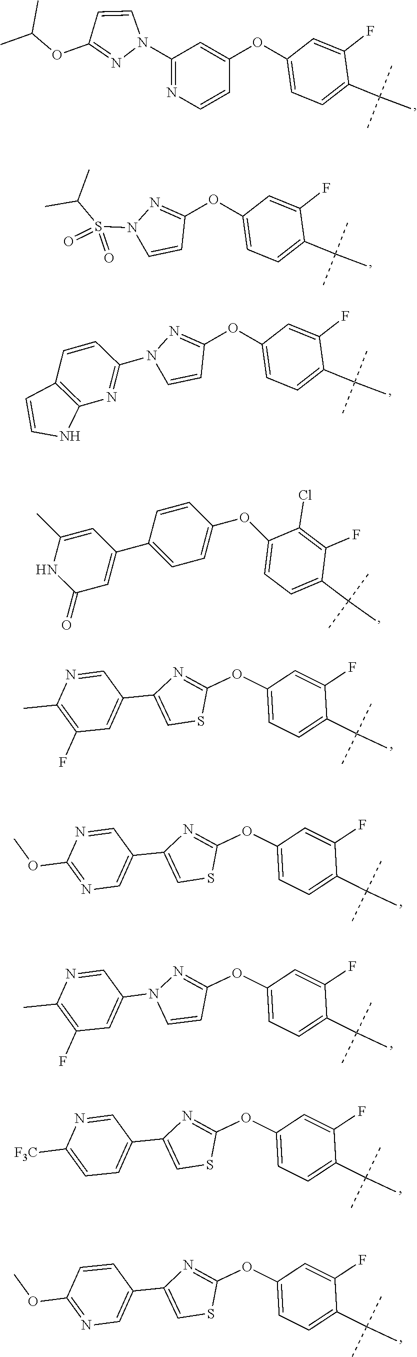

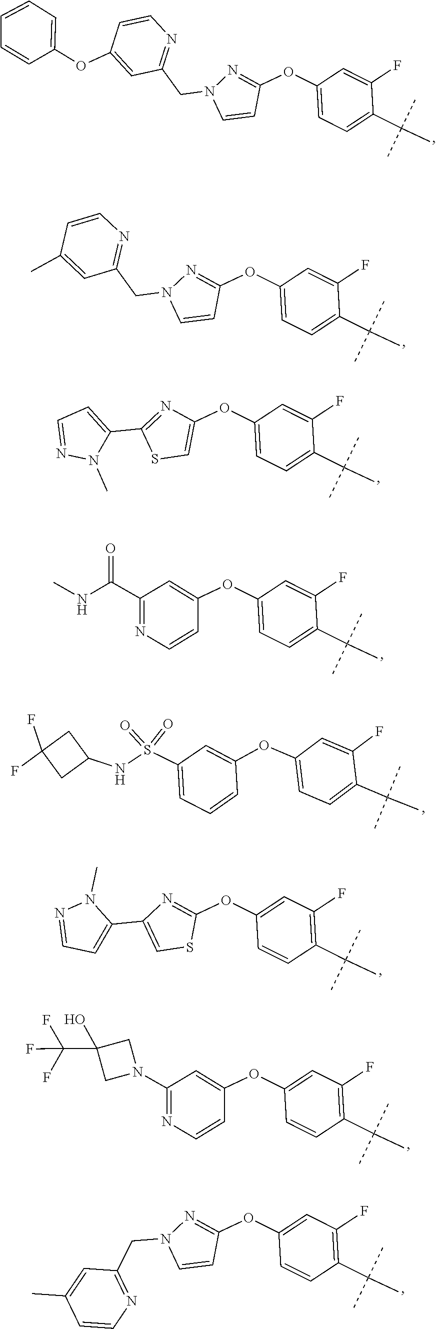

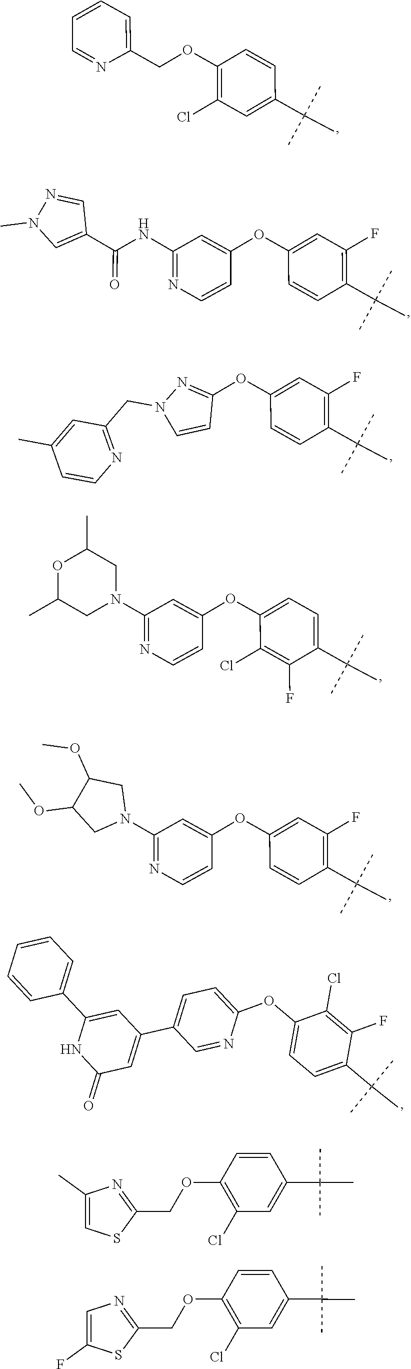

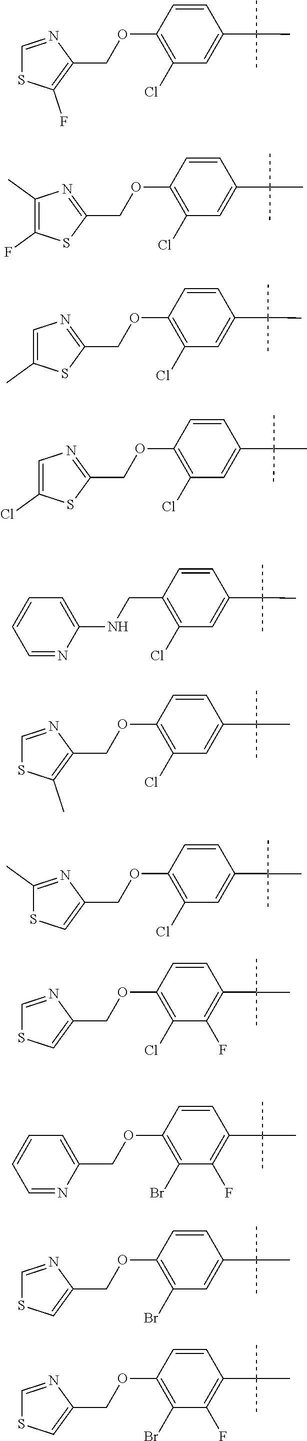

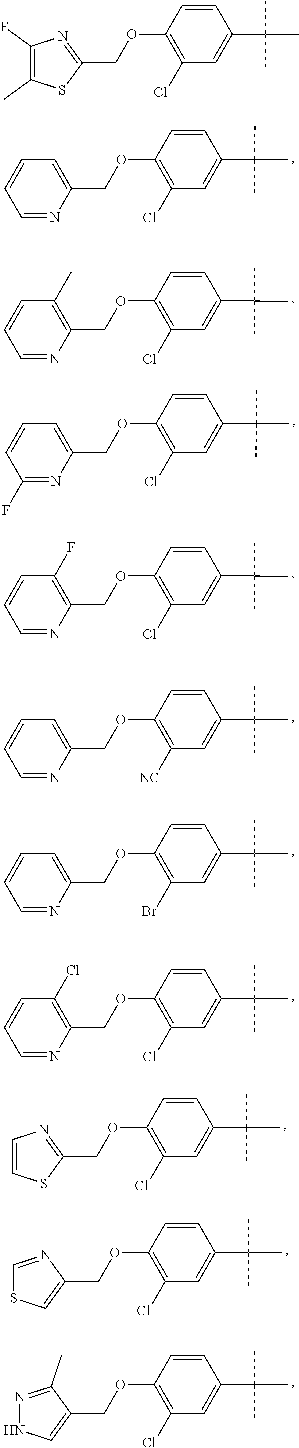

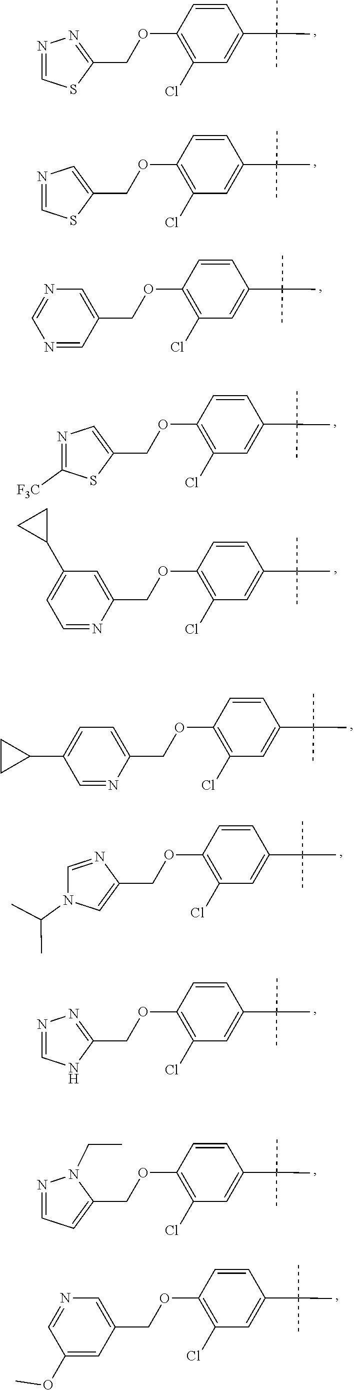

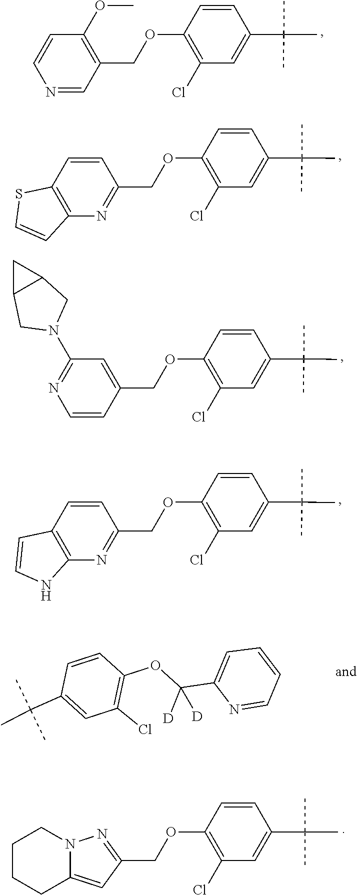

In some embodiments, R₂ is chosen from

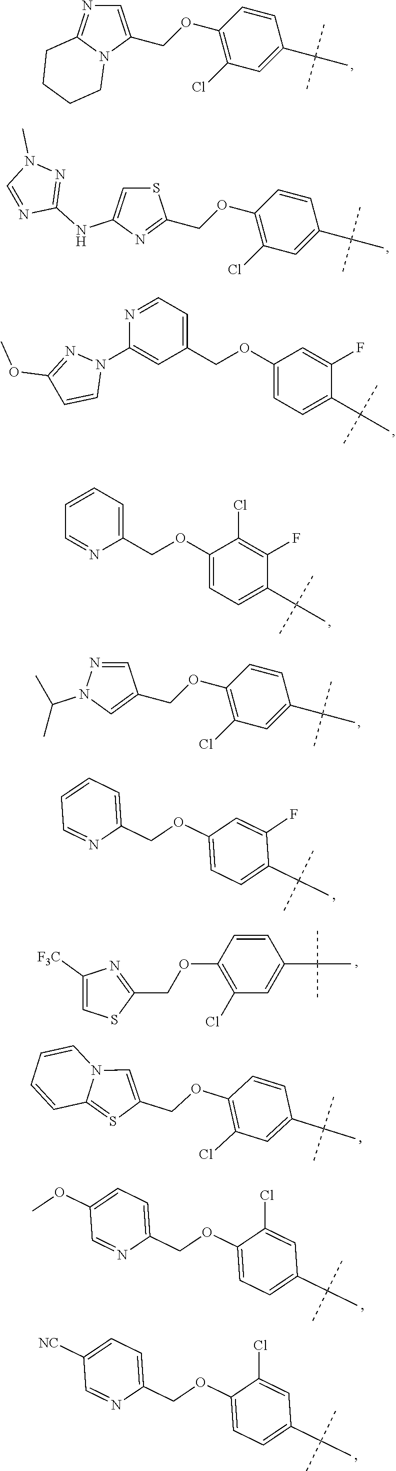

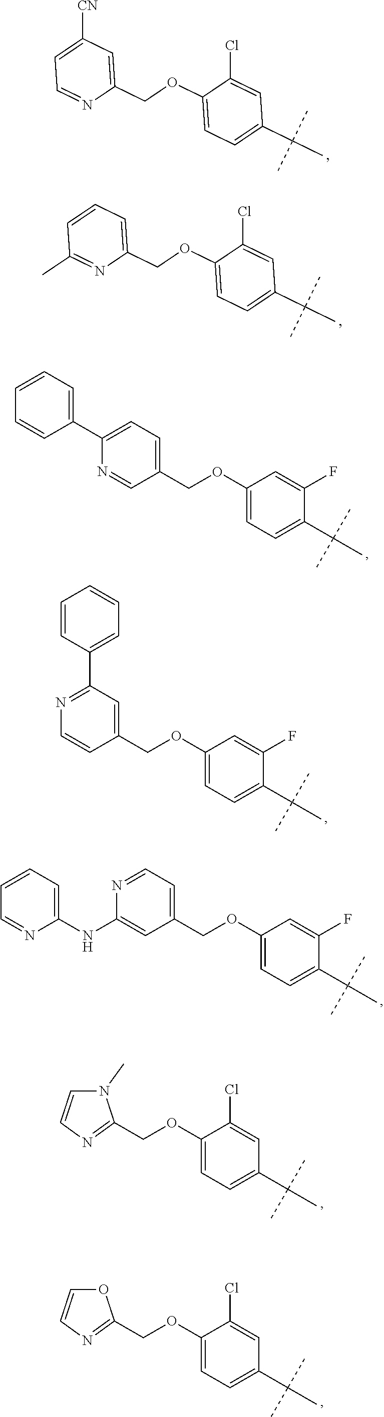

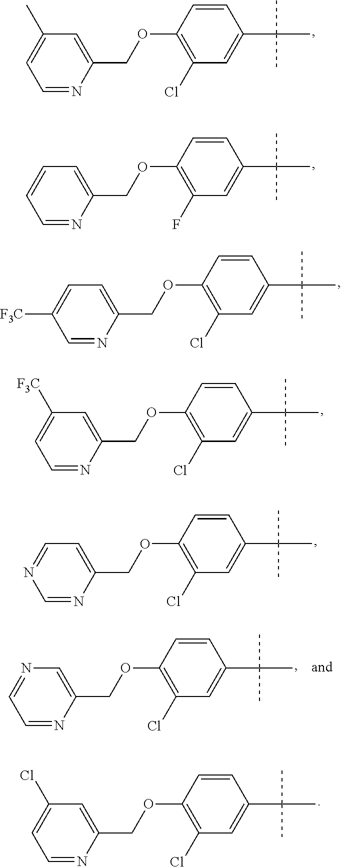

In some embodiments, R₂ is chosen from

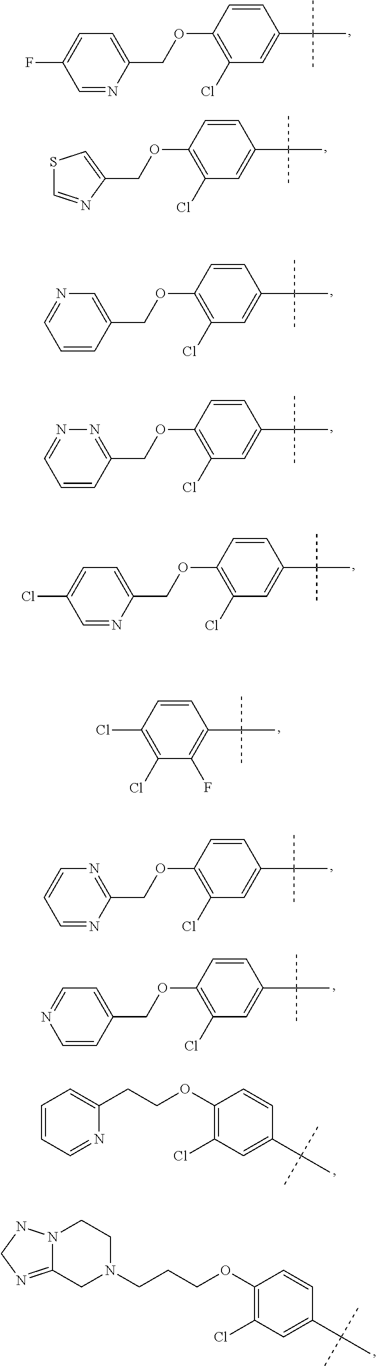

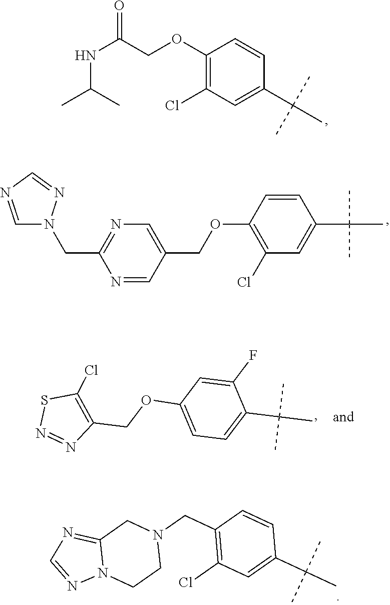

In some embodiments, R₂ is chosen from

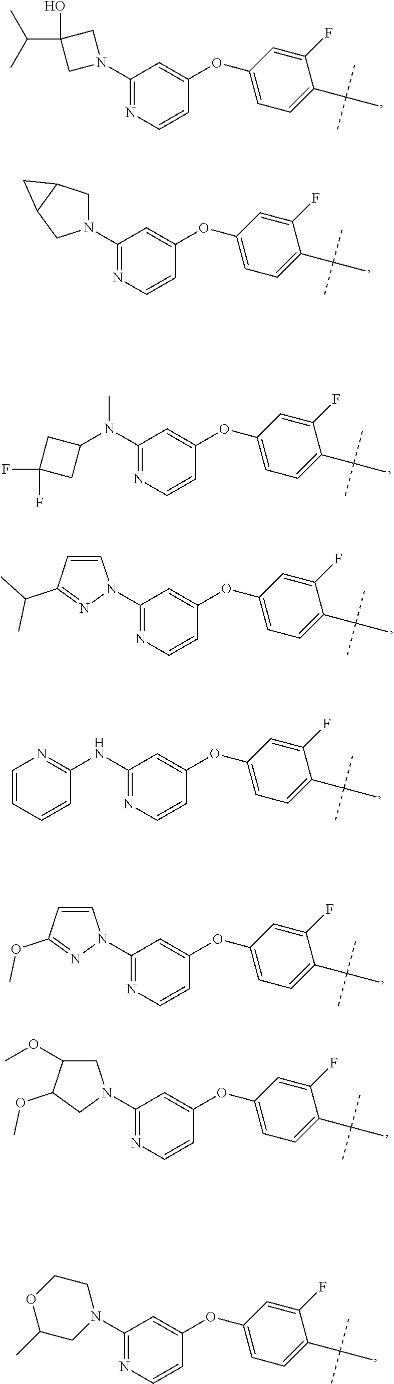

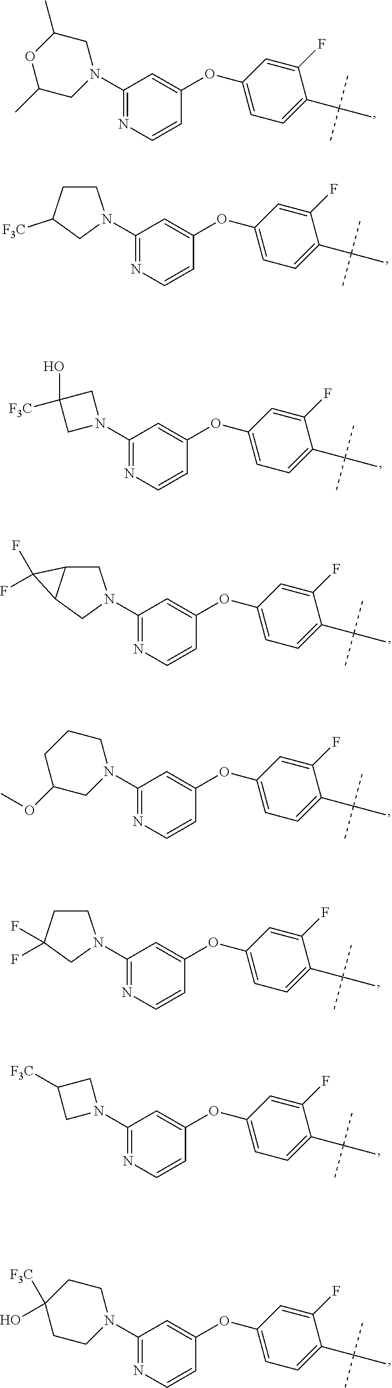

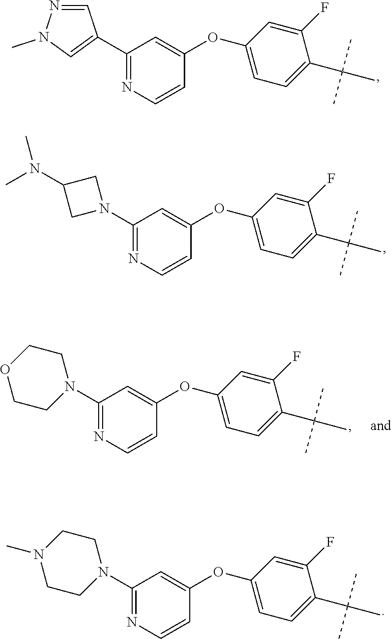

In some embodiments, R₂ is chosen from

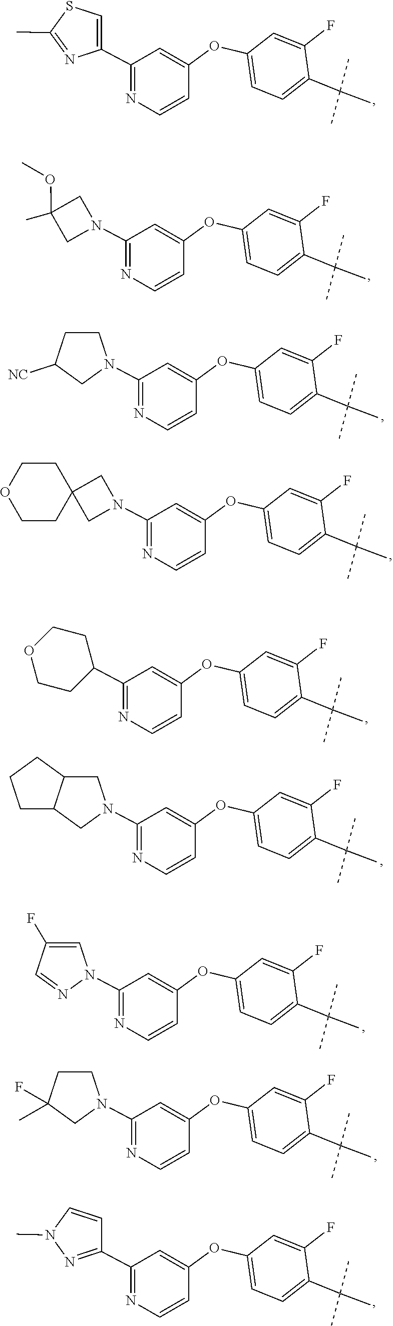

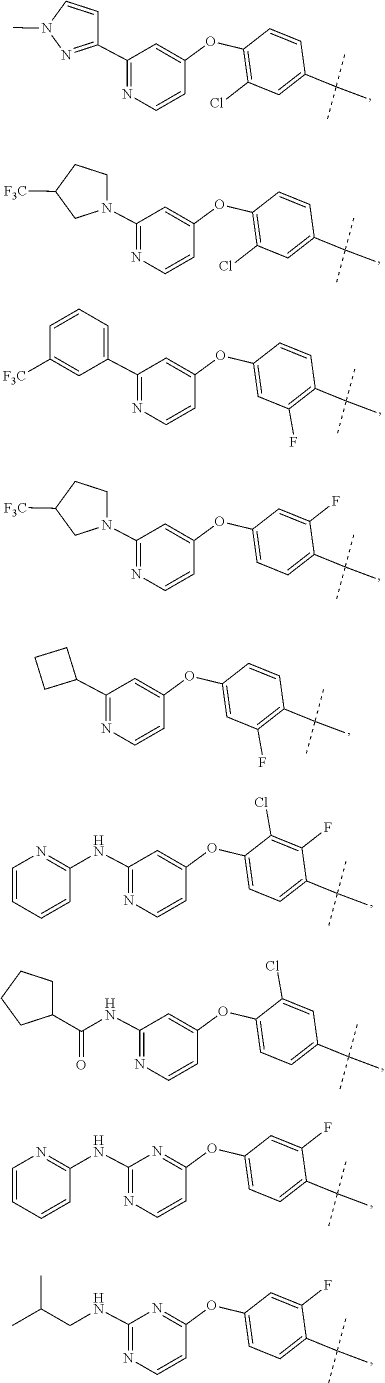

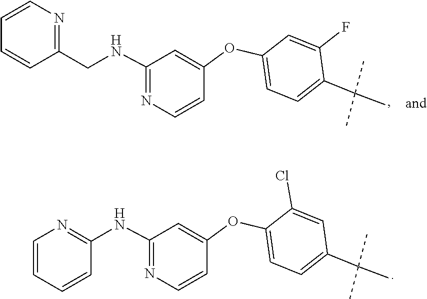

In some embodiments, R₂ is chosen from

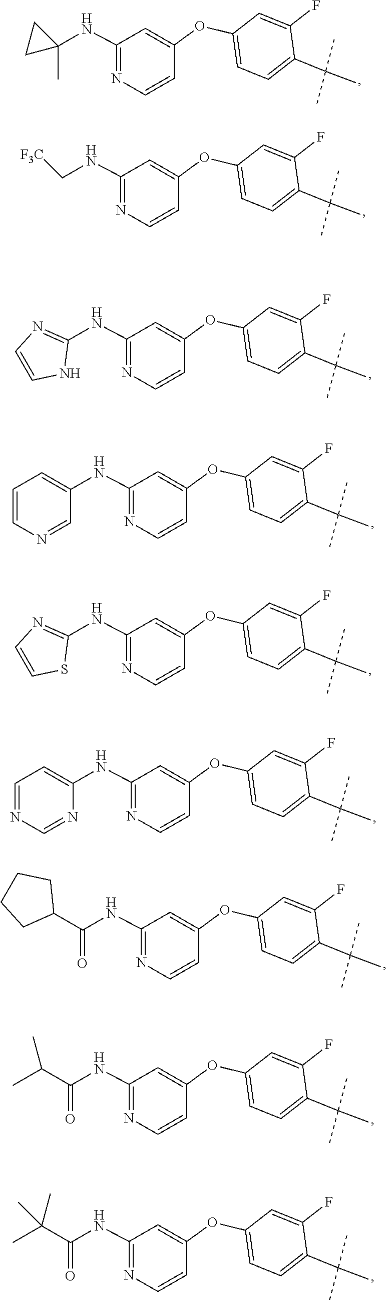

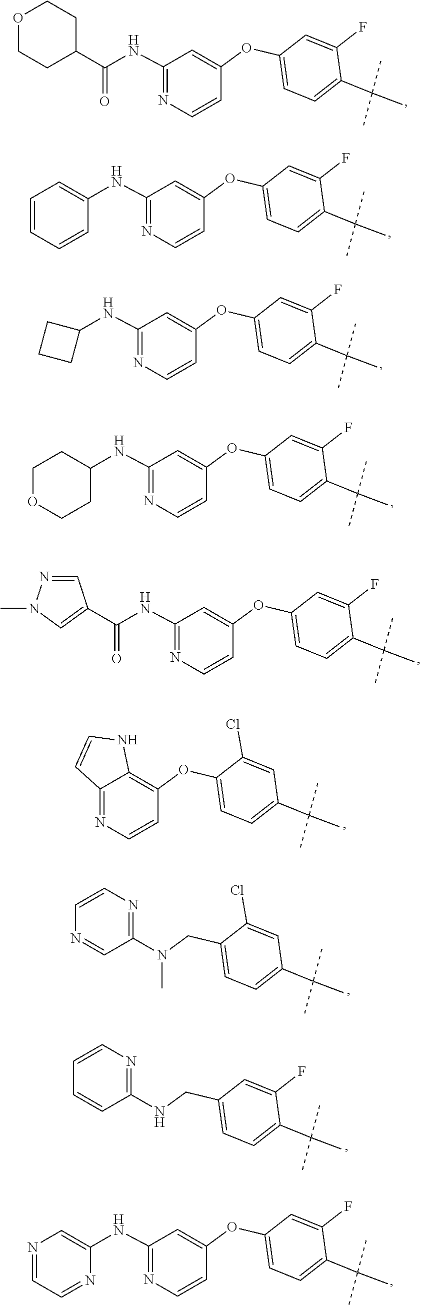

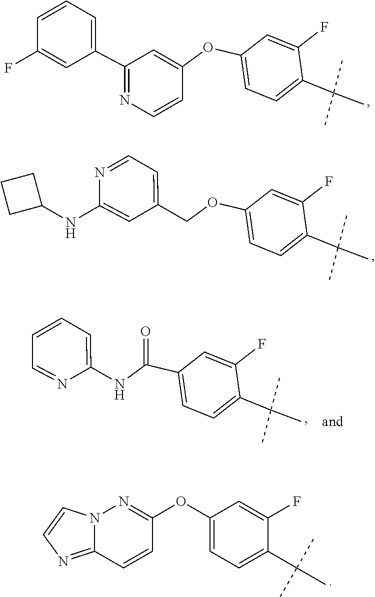

In some embodiments, R₂ is chosen from

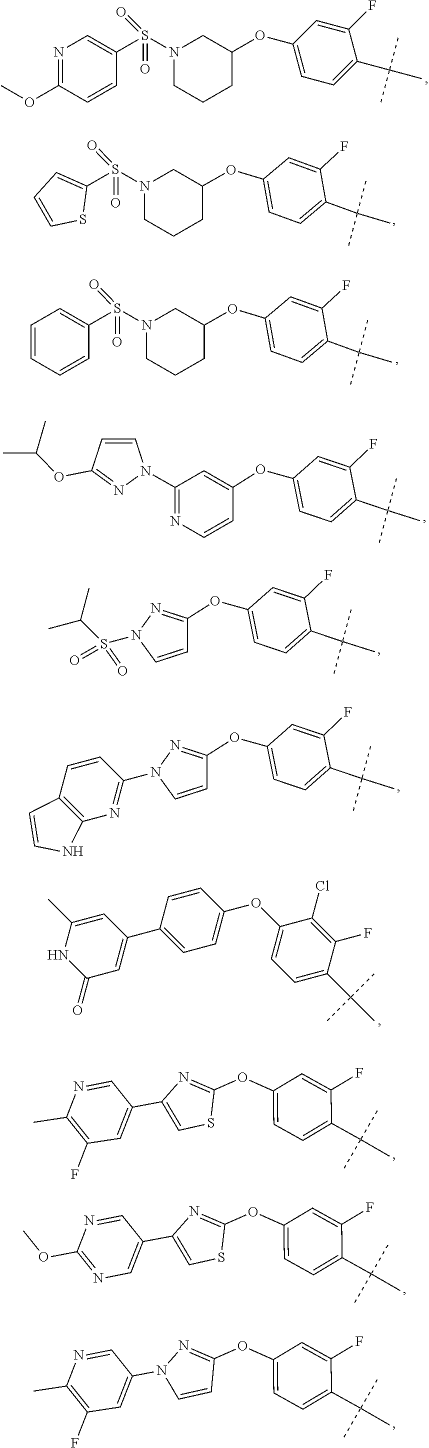

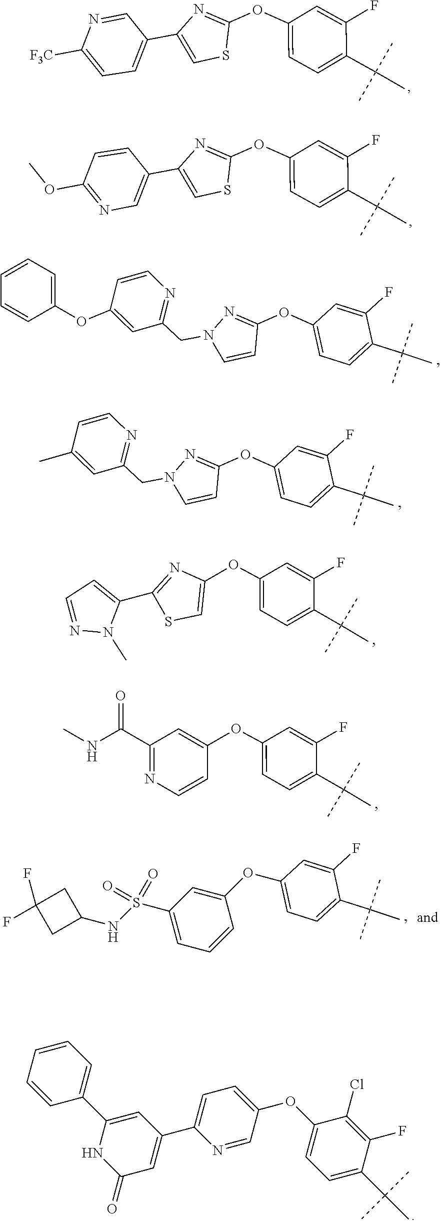

In some embodiments, R₂ is chosen from

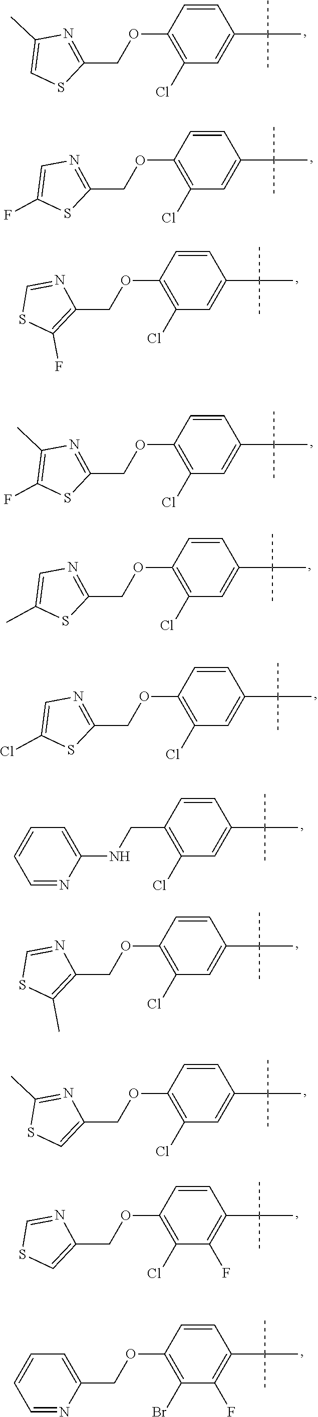

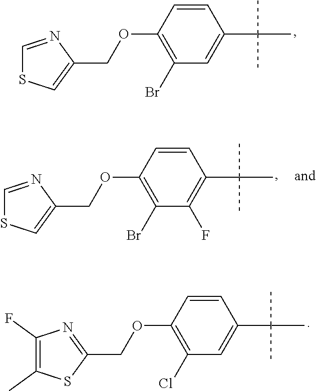

In some embodiments, provided herein is a compound, or pharmaceutically acceptable salt thereof, chosen from the compounds listed in Table 1.

TABLE 1
Exemplary Compounds of the Present Disclosure

| Compound | | |
|---|---|---|
| No. | Chemical Structure | IUPAC Name |
| 1 | 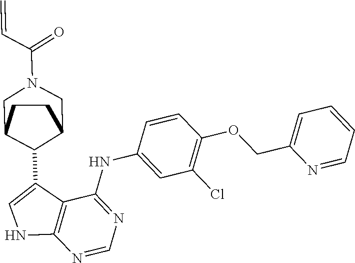 | (8-syn)-8-(4-((3-chloro-4-(pyridin-2- ylmethoxy)phenyl)amino)-7H-pyrrolo[2,3- d]pyrimidin-5-yl)-3-azabicyclo[3.2.1]octan-3- yl)prop-2-en-1-one |
| 2 | 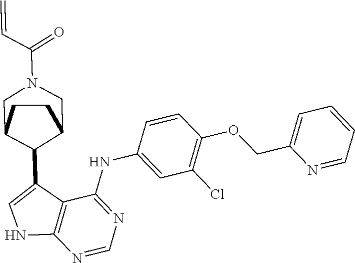 | (8-anti)-8-(4-((3-chloro-4-(pyridin-2- ylmethoxy)phenyl)amino)-7H-pyrrolo[2,3- d]pyrimidin-5-yl)-3-azabicyclo[3.2.1]octan-3- yl)prop-2-en-1-one |
| 3 | 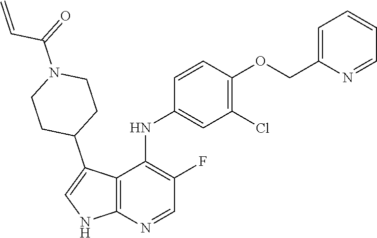 [SMILES: C=CC(=O)N1CCC(c2c[nH]c3ncc(F)c(Nc4ccc(OCc5ccccn5)c(Cl)c4)c23)CC1] | 1-(4-(4-((3-chloro-4-(pyridin-2- ylmethoxy)phenyl)amino)-5-fluoro-1H- pyrrolo[2,3-b]pyridin-3-yl)piperidin-1-yl)prop- 2-en-1-one |
| 4 | 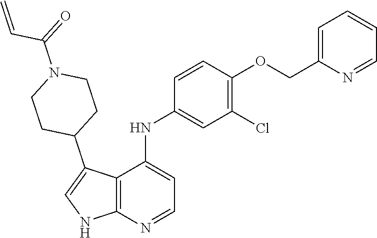 [SMILES: C=CC(=O)N1CCC(c2c[nH]c3nccc(Nc4ccc(OCc5ccccn5)c(Cl)c4)c23)CC1] | 1-(4-(4-((3-chloro-4-(pyridin-2- ylmethoxy)phenyl)amino)-1H-pyrrolo[2,3- b]pyridin-3-yl)piperidin-1-yl)prop-2-en-1-one |
| 5 | 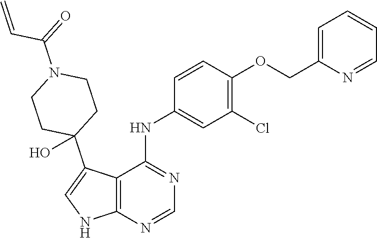 [SMILES: C=CC(=O)N1CCC(O)(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)-4-hydroxypiperidin-1- yl)prop-2-en-1-one |
| 6 | 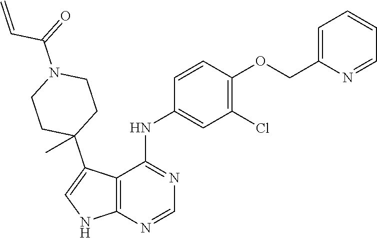 [SMILES: C=CC(=O)N1CCC(C)(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)-4-methylpiperidin-1-yl)prop- 2-en-1-one |
| 7 | 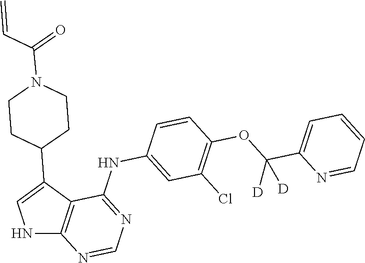 [SMILES: [2H]C([2H])(Oc1ccc(Nc2ncnc3[nH]cc(C4CCN(C(=O)C=C)CC4)c23)cc1Cl)c1ccccn1] | 1-(4-(4-((3-chloro-4-(pyridin-2-ylmethoxy- d2)phenyl)amino)-7H-pyrrolo[2,3- d]pyrimidin-5-yl)piperidin-1-yl)prop-2-en-1- one |
| 8 | 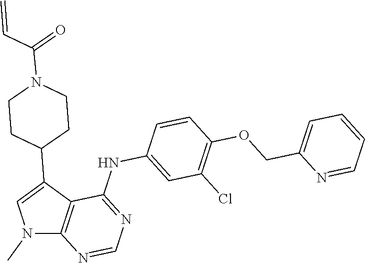 [SMILES: C=CC(=O)N1CCC(c2cn(C)c3ncnc(Nc4ccc(OCc5ccccn5)c(Cl)c4)c23)CC1] | 1-(4-(4-((3-chloro-4-(pyridin-2- ylmethoxy)phenyl)amino)-7-methyl-7H- pyrrolo[2,3-d]pyrimidin-5-yl)piperidin-1- yl)prop-2-en-1-one |
| 9 | 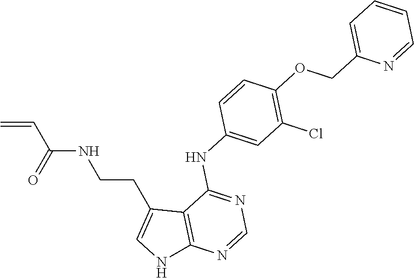 [SMILES: C=CC(=O)NCCc1c[nH]c2ncnc(Nc3ccc(OCc4ccccn4)c(Cl)c3)c12] | N-(2-(4-((3-chloro-4-(pyridin-2- ylmethoxy)phenyl)amino)-7H-pyrrolo[2,3- d]pyrimidin-5-yl)ethyl)acrylamide |
| 10 | 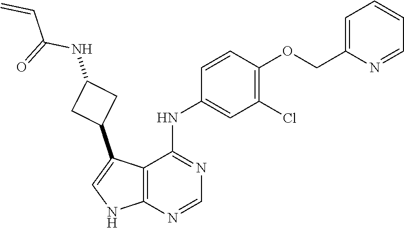 | N-((1,3-trans)-3-(4-((3-chloro-4-(pyridin-2- ylmethoxy)phenyl)amino)-7H-pyrrolo[2,3- d]pyrimidin-5-yl)cycloButyl)acrylamide |
| 11 | 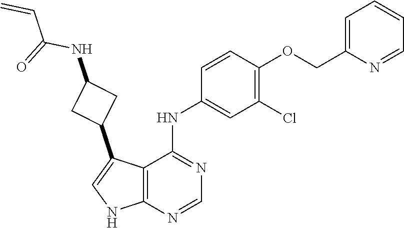 | N-((1,3-cis)-3-(4-((3-chloro-4-(pyridin-2- ylmethoxy)phenyl)amino)-7H-pyrrolo[2,3- d]pyrimidin-5-yl)cycloButyl)acrylamide |
| 12 | 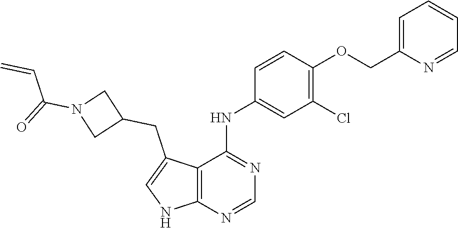 [SMILES: C=CC(=O)N1CC(Cc2c[nH]c3ncnc(Nc4ccc(OCc5ccccn5)c(Cl)c4)c23)C1] | 1-(3-((4-((3-chloro-4-(pyridin-2- ylmethoxy)phenyl)amino)-7H-pyrrolo[2,3- d]pyrimidin-5-yl)methyl)azetidin-1-yl)prop-2- en-1-one |
| 13 | 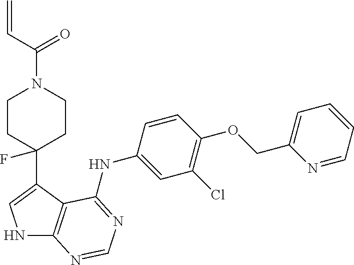 [SMILES: C=CC(=O)N1CCC(F)(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)-4-fluoropiperidin-1-yl)prop- 2-en-1-one |
| 14 | 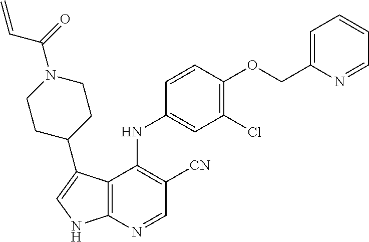 [SMILES: C=CC(=O)N1CCC(c2c[nH]c3ncc(C#N)c(Nc4ccc(OCc5ccccn5)c(Cl)c4)c23)CC1] | 3-(1-acryloylpiperidin-4-yl)-4-((3-chloro-4- (pyridin-2-ylmethoxy)phenyl)amino)-1H- pyrrolo[2,3-b]pyridine-5-carbonitrile |
| 15 | 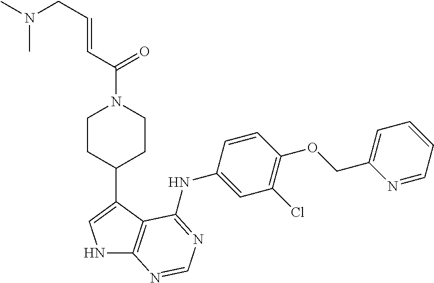 [SMILES: CN(C)C/C=C/C(=O)N1CCC(c2c[nH]c3ncnc(Nc4ccc(OCc5ccccn5)c(Cl)c4)c23)CC1] | (E)-1-(4-(4-((3-chloro-4-(pyridin-2- ylmethoxy)phenyl)amino)-7H-pyrrolo[2,3- d]pyrimidin-5-yl)piperidin-1-yl)-4- (dimethylamino)but-2-en-1-one |
| 16 | 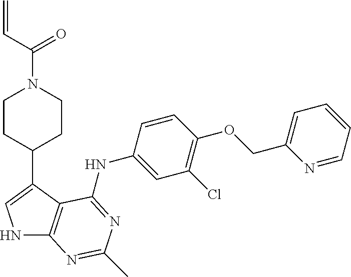 [SMILES: C=CC(=O)N1CCC(c2c[nH]c3nc(C)nc(Nc4ccc(OCc5ccccn5)c(Cl)c4)c23)CC1] | 1-(4-(4-((3-chloro-4-(pyridin-2- ylmethoxy)phenyl)amino)-2-methyl-7H- pyrrolo[2,3-d]pyrimidin-5-yl)piperidin-1- yl)prop-2-en-1-one |
| 17 | 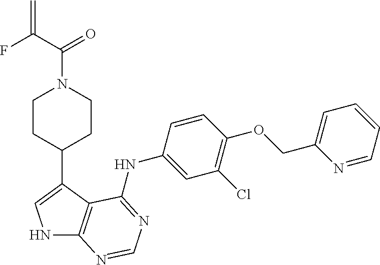 [SMILES: C=C(F)C(=O)N1CCC(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)piperidin-1-yl)-2-fluoroprop- 2-en-1-one |
| 18 | 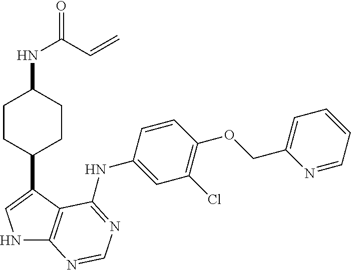 | N-((1,4-ciss)-4-(4-((3-chloro-4-(pyridin-2- ylmethoxy)phenyl)amino)-7H-pyrrolo[2,3- d]pyrimidin-5-yl)cyclohexyl)acrylamide |
| 19 | 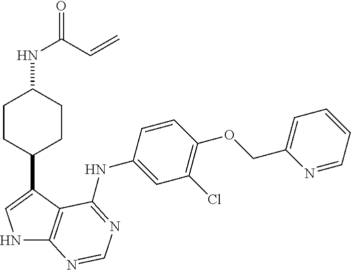 | N-((1,4-trans)-4-(4-((3-chloro-4-(pyridin-2- ylmethoxy)phenyl)amino)-7H-pyrrolo[2,3- d]pyrimidin-5-yl)cyclohexyl)acrylamide |
| 20 | 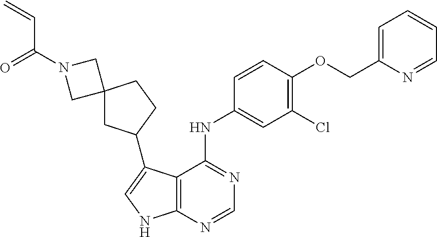 [SMILES: C=CC(=O)N1CC2(CCC(c3c[nH]c4ncnc(Nc5ccc(OCc6ccccn6)c(Cl)c5)c34)C2)C1] | 1-(6-(4-((3-chloro-4-(pyridin-2- ylmethoxy)phenyl)amino)-7H-pyrrolo[2,3- d]pyrimidin-5-yl)-2-azaspiro[3.4]octan-2- yl)prop-2-en-1-one |
| 21 | 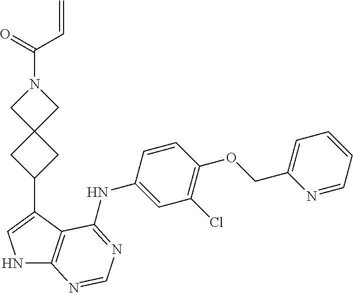 [SMILES: C=CC(=O)N1CC2(CC(c3c[nH]c4ncnc(Nc5ccc(OCc6ccccn6)c(Cl)c5)c34)C2)C1] | 1-(6-(4-((3-chloro-4-(pyridin-2- ylmethoxy)phenyl)amino)-7H-pyrrolo[2,3- d]pyrimidin-5-yl)-2-azaspiro[3.3]heptan-2- yl)prop-2-en-1-one |
| 22 | 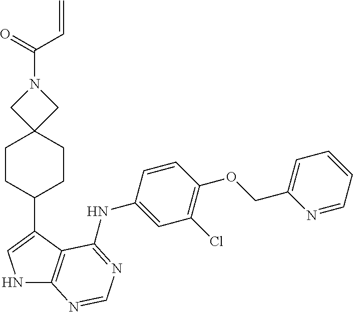 [SMILES: C=CC(=O)N1CC2(CCC(c3c[nH]c4ncnc(Nc5ccc(OCc6ccccn6)c(Cl)c5)c34)CC2)C1] | 1-(7-(4-((3-chloro-4-(pyridin-2- ylmethoxy)phenyl)amino)-7H-pyrrolo[2,3- d]pyrimidin-5-yl)-2-azaspiro[3.5]nonan-2- yl)prop-2-en-1-one |
| 23 | 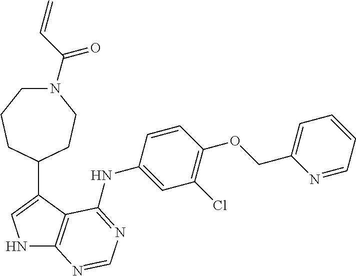 [SMILES: C=CC(=O)N1CCCC(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)azepan-1-yl)prop-2-en-1- one |
| 24 | 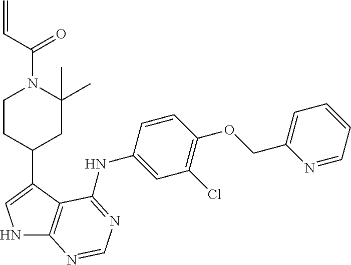 [SMILES: C=CC(=O)N1CCC(c2c[nH]c3ncnc(Nc4ccc(OCc5ccccn5)c(Cl)c4)c23)CC1(C)C] | 1-(4-(4-((3-chloro-4-(pyridin-2- ylmethoxy)phenyl)amino)-7H-pyrrolo[2,3- d]pyrimidin-5-yl)-2,2-dimethylpiperidin-1- yl)prop-2-en-1-one |
| 25 | 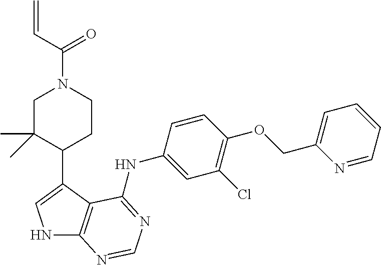 [SMILES: C=CC(=O)N1CCC(c2c[nH]c3ncnc(Nc4ccc(OCc5ccccn5)c(Cl)c4)c23)C(C)(C)C1] | 1-(4-(4-((3-chloro-4-(pyridin-2- ylmethoxy)phenyl)amino)-7H-pyrrolo[2,3- d]pyrimidin-5-yl)-3,3-dimethylpiperidin-1- yl)prop-2-en-1-one |
| 26 | 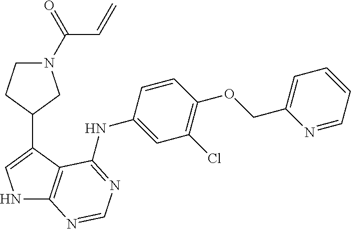 [SMILES: C=CC(=O)N1CCC(c2c[nH]c3ncnc(Nc4ccc(OCc5ccccn5)c(Cl)c4)c23)C1] | 1-(3-(4-((3-chloro-4-(pyridin-2- ylmethoxy)phenyl)amino)-7H-pyrrolo[2,3- d]pyrimidin-5-yl)pyrrolidin-1-yl)prop-2-en-1- one |
| 27 | 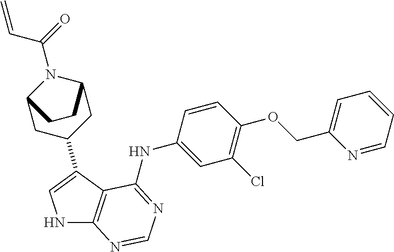 | 3-(4-((3-chloro-4-(pyridin-2- ylmethoxy)phenyl)amino)-7H-pyrrolo[2,3- d]pyrimidin-5-yl)-3-exo-8- azabicyclo[3.2.1]octan-8-yl)prop-2-en-1-one |
| 28 | 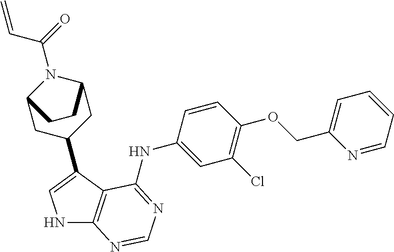 | 3-(4-((3-chloro-4-(pyridin-2- ylmethoxy)phenyl)amino)-7H-pyrrolo[2,3- d]pyrimidin-5-yl)-3-endo-8- azabicyclo[3.2.1]octan-8-yl)prop-2-en-1-one |
| 29 | 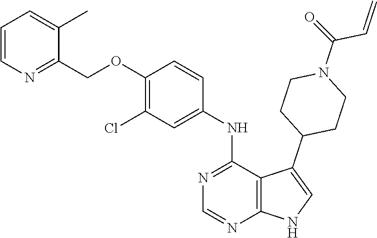 [SMILES: C=CC(=O)N1CCC(c2c[nH]c3ncnc(Nc4ccc(OCc5ncccc5C)c(Cl)c4)c23)CC1] | 1-(4-(4-((3-chloro-4-((3-methylpyridin-2- yl)methoxy)phenyl)amino)-7H-pyrrolo[2,3- d]pyrimidin-5-yl)piperidin-1-yl)prop-2-en-1- one |
| 30 | 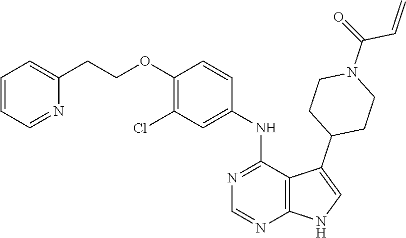 [SMILES: C=CC(=O)N1CCC(c2c[nH]c3ncnc(Nc4ccc(OCCc5ccccn5)c(Cl)c4)c23)CC1] | 1-(4-(4-((3-chloro-4-(2-(pyridin-2- yl)ethoxy)phenyl)amino)-7H-pyrrolo[2,3- d]pyrimidin-5-yl)piperidin-1-yl)prop-2-en-1- one |
| 31 | 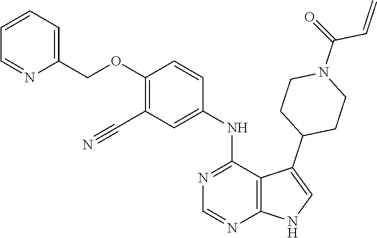 [SMILES: C=CC(=O)N1CCC(c2c[nH]c3ncnc(Nc4ccc(OCc5ccccn5)c(C#N)c4)c23)CC1] | 5-((5-(1-acryloylpiperidin-4-yl)-7H- pyrrolo[2,3-d]pyrimidin-4-yl)amino)-2- (pyridin-2-ylmethoxy)benzonitrile |
| 32 | 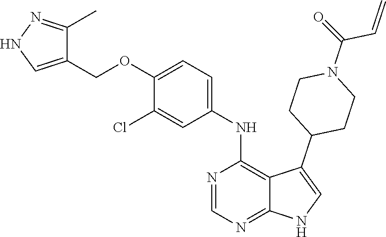 [SMILES: C=CC(=O)N1CCC(c2c[nH]c3ncnc(Nc4ccc(OCc5c[nH]nc5C)c(Cl)c4)c23)CC1] | 1-(4-(4-((3-chloro-4-((3-methyl-1H-pyrazol-4- yl)methoxy)phenyl)amino)-7H-pyrrolo[2,3- d]pyrimidin-5-yl)piperidin-1-yl)prop-2-en-1- one |
| 33 | 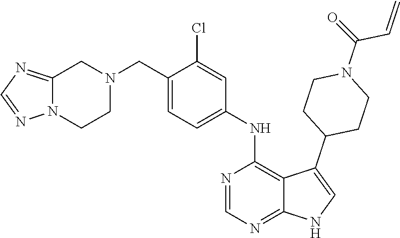 [SMILES: C=CC(=O)N1CCC(c2c[nH]c3ncnc(Nc4ccc(CN5CCn6ncnc6C5)c(Cl)c4)c23)CC1] | 1-(4-(4-((3-chloro-4-((5,6-dihydro- [1,2,4]triazolo[1,5-a]pyrazin-7(8H)- yl)methyl)phenyl)amino)-7H-pyrrolo[2,3- d]pyrimidin-5-yl)piperidin-1-yl)prop-2-en-1- one |
| 34 | 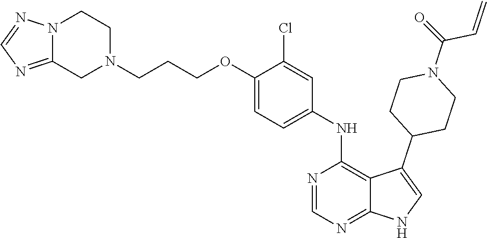 [SMILES: C=CC(=O)N1CCC(c2c[nH]c3ncnc(Nc4ccc(OCCCN5CCn6ncnc6C5)c(Cl)c4)c23)CC1] | 1-(4-(4-((3-chloro-4-(3-(5,6-dihydro- [1,2,4]triazolo[1,5-a]pyrazin-7(8H)- yl)propoxy)phenyl)amino)-7H-pyrrolo[2,3- d]pyrimidin-5-yl)piperidin-1-yl)prop-2-en-1- one |
| 35 | 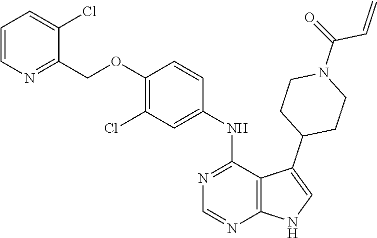 [SMILES: C=CC(=O)N1CCC(c2c[nH]c3ncnc(Nc4ccc(OCc5ncccc5Cl)c(Cl)c4)c23)CC1] | 1-(4-(4-((3-chloro-4-((3-chloropyridin-2- yl)methoxy)phenyl)amino)-7H-pyrrolo[2,3- d]pyrimidin-5-yl)piperidin-1-yl)prop-2-en-1- one |
| 36 | 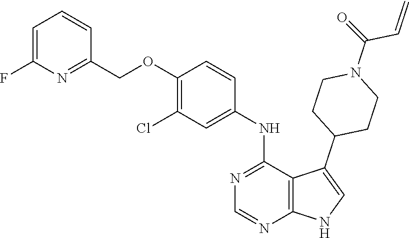 [SMILES: C=CC(=O)N1CCC(c2c[nH]c3ncnc(Nc4ccc(OCc5cccc(F)n5)c(Cl)c4)c23)CC1] | 1-(4-(4-((3-chloro-4-((6-fluoropyridin-2- yl)methoxy)phenyl)amino)-7H-pyrrolo[2,3- d]pyrimidin-5-yl)piperidin-1-yl)prop-2-en-1- one |
| 37 | 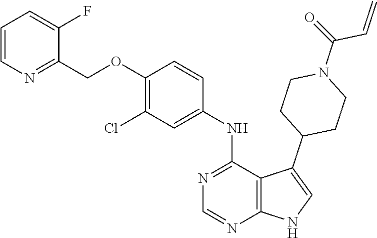 [SMILES: C=CC(=O)N1CCC(c2c[nH]c3ncnc(Nc4ccc(OCc5ncccc5F)c(Cl)c4)c23)CC1] | 1-(4-(4-((3-chloro-4-((3-fluoropyridin-2- yl)methoxy)phenyl)amino)-7H-pyrrolo[2,3- d]pyrimidin-5-yl)piperidin-1-yl)prop-2-en-1- one |
| 38 | 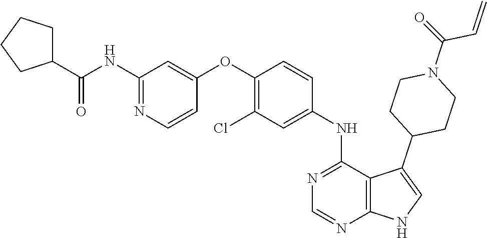 [SMILES: C=CC(=O)N1CCC(c2c[nH]c3ncnc(Nc4ccc(Oc5ccnc(NC(=O)C6CCCC6)c5)c(Cl)c4)c23)CC1] | N-(4-(4-((5-(1-acryloylpiperidin-4-yl)-7H- pyrrolo[2,3-d]pyrimidin-4-yl)amino)-2- chlorophenoxy)pyridin-2- yl)cyclopentanecarboxamide |
| 39 | 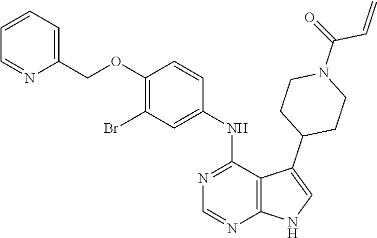 [SMILES: C=CC(=O)N1CCC(c2c[nH]c3ncnc(Nc4ccc(OCc5ccccn5)c(Br)c4)c23)CC1] | 1-(4-(4-((3-bromo-4-(pyridin-2- ylmethoxy)phenyl)amino)-7H-pyrrolo[2,3- d]pyrimidin-5-yl)piperidin-1-yl)prop-2-en-1- one |
| 40 | 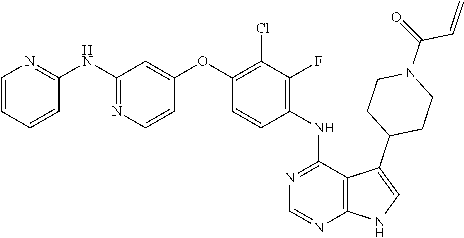 [SMILES: C=CC(=O)N1CCC(c2c[nH]c3ncnc(Nc4ccc(Oc5ccnc(Nc6ccccn6)c5)c(Cl)c4F)c23)CC1] | 1-(4-(4-((3-chloro-2-fluoro-4-((2-(pyridin-2- ylamino)pyridin-4-yl)oxy)phenyl)amino)-7H- pyrrolo[2,3-d]pyrimidin-5-yl)piperidin-1- yl)prop-2-en-1-one |
| 41 | 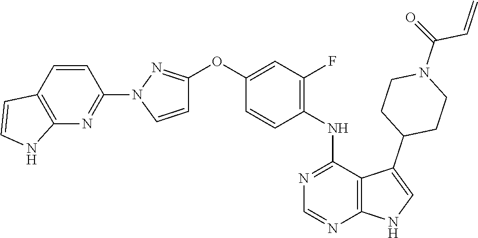 [SMILES: C=CC(=O)N1CCC(c2c[nH]c3ncnc(Nc4ccc(Oc5ccn(-c6ccc7cc[nH]c7n6)n5)cc4F)c23)CC1] | 1-(4-(4-((4-((1-(1H-pyrrolo[2,3-b]pyridin-6- yl)-1H-pyrazol-3-yl)oxy)-2- fluorophenyl)amino)-7H-pyrrolo[2,3- d]pyrimidin-5-yl)piperidin-1-yl)prop-2-en-1- one |
| 42 | 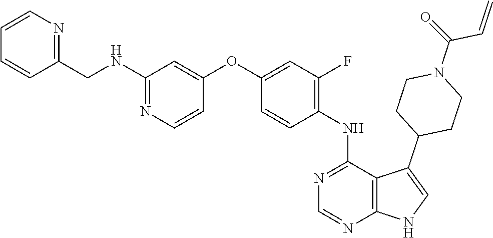 [SMILES: C=CC(=O)N1CCC(c2c[nH]c3ncnc(Nc4ccc(Oc5ccnc(NCc6ccccn6)c5)cc4F)c23)CC1] | 1-(4-(4-((2-fluoro-4-((2-((pyridin-2- ylmethyl)amino)pyridin-4- yl)oxy)phenyl)amino)-7H-pyrrolo[2,3- d]pyrimidin-5-yl)piperidin-1-yl)prop-2-en-1- one |
| 43 | 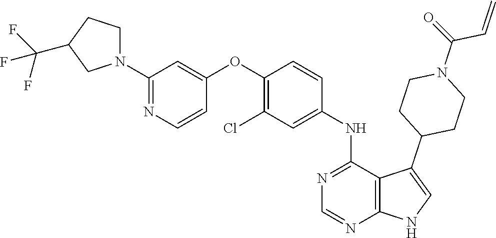 [SMILES: C=CC(=O)N1CCC(c2c[nH]c3ncnc(Nc4ccc(Oc5ccnc(N6CCC(C(F)(F)F)C6)c5)c(Cl)c4)c23)CC1] | 1-(4-(4-((3-chloro-4-((2-(3- (trifluoromethyl)pyrrolidin-1-yl)pyridin-4- yl)oxy)phenyl)amino)-7H-pyrrolo[2,3- d]pyrimidin-5-yl)piperidin-1-yl)prop-2-en-1- one |
| 44 | 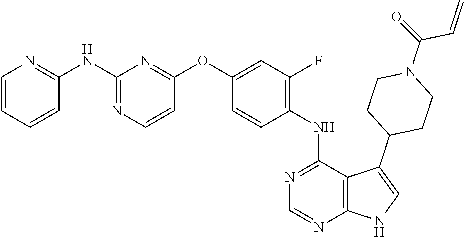 [SMILES: C=CC(=O)N1CCC(c2c[nH]c3ncnc(Nc4ccc(Oc5ccnc(Nc6ccccn6)n5)cc4F)c23)CC1] | 1-(4-(4-((2-fluoro-4-((2-(pyridin-2- ylamino)pyrimidin-4-yl)oxy)phenyl)amino)- 7H-pyrrolo[2,3-d]pyrimidin-5-yl)piperidin-1- yl)prop-2-en-1-one |
| 45 | 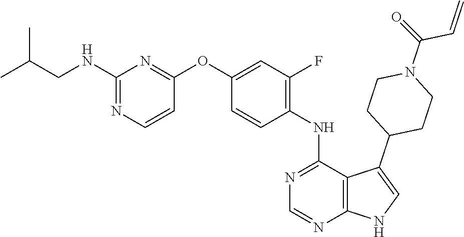 [SMILES: C=CC(=O)N1CCC(c2c[nH]c3ncnc(Nc4ccc(Oc5ccnc(NCC(C)C)n5)cc4F)c23)CC1] | 1-(4-(4-((2-fluoro-4-((2- (isoButylamino)pyrimidin-4- yl)oxy)phenyl)amino)-7H-pyrrolo[2,3- d]pyrimidin-5-yl)piperidin-1-yl)prop-2-en-1- one |
| 46 | 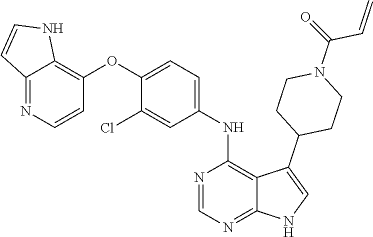 [SMILES: C=CC(=O)N1CCC(c2c[nH]c3ncnc(Nc4ccc(Oc5ccnc6cc[nH]c56)c(Cl)c4)c23)CC1] | 1-(4-(4-((4-((1H-pyrrolo[3,2-b]pyridin-7- yl)oxy)-3-chlorophenyl)amino)-7H- pyrrolo[2,3-d]pyrimidin-5-yl)piperidin-1- yl)prop-2-en-1-one |
| 47 | 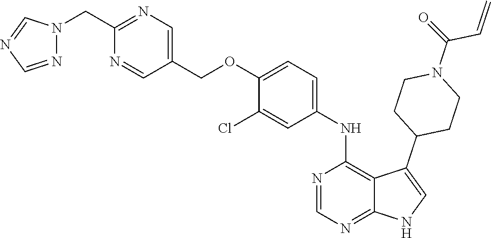 [SMILES: C=CC(=O)N1CCC(c2c[nH]c3ncnc(Nc4ccc(OCc5cnc(Cn6cncn6)nc5)c(Cl)c4)c23)CC1] | 1-(4-(4-((4-((2-((1H-1,2,4-triazol-1- yl)methyl)pyrimidin-5-yl)methoxy)-3- chlorophenyl)amino)-7H-pyrrolo[2,3- d]pyrimidin-5-yl)piperidin-1-yl)prop-2-en-1- one |
| 48 | 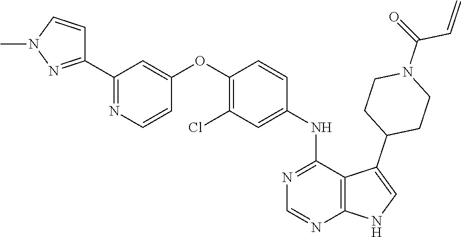 [SMILES: C=CC(=O)N1CCC(c2c[nH]c3ncnc(Nc4ccc(Oc5ccnc(-c6ccn(C)n6)c5)c(Cl)c4)c23)CC1] | 1-(4-(4-((3-chloro-4-((2-(1-methyl-1H- pyrazol-3-yl)pyridin-4-yl)oxy)phenyl)amino)- 7H-pyrrolo[2,3-d]pyrimidin-5-yl)piperidin-1- yl)prop-2-en-1-one |
| 49 | 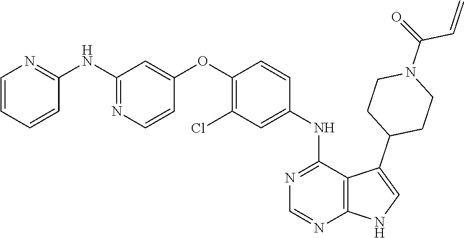 [SMILES: C=CC(=O)N1CCC(c2c[nH]c3ncnc(Nc4ccc(Oc5ccnc(Nc6ccccn6)c5)c(Cl)c4)c23)CC1] | 1-(4-(4-((3-chloro-4-((2-(pyridin-2- ylamino)pyridin-4-yl)oxy)phenyl)amino)-7H- pyrrolo[2,3-d]pyrimidin-5-yl)piperidin-1- yl)prop-2-en-1-one |
| 50 | 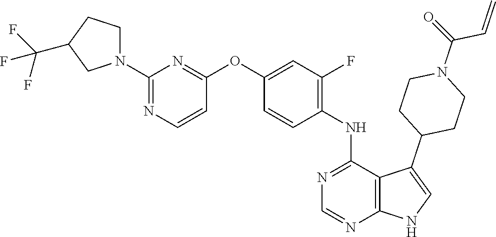 [SMILES: C=CC(=O)N1CCC(c2c[nH]c3ncnc(Nc4ccc(Oc5ccnc(N6CCC(C(F)(F)F)C6)n5)cc4F)c23)CC1] | 1-(4-(4-((2-fluoro-4-((2-(3- (trifluoromethyl)pyrrolidin-1-yl)pyrimidin-4- yl)oxy)phenyl)amino)-7H-pyrrolo[2,3- d]pyrimidin-5-yl)piperidin-1-yl)prop-2-en-1- one |
| 51 | 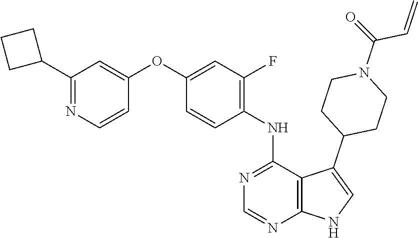 [SMILES: C=CC(=O)N1CCC(c2c[nH]c3ncnc(Nc4ccc(Oc5ccnc(C6CCC6)c5)cc4F)c23)CC1] | 1-(4-(4-((4-((2-cycloButylpyridin-4-yl)oxy)-2- fluorophenyl)amino)-7H-pyrrolo[2,3- d]pyrimidin-5-yl)piperidin-1-yl)prop-2-en-1- one |
| 52 | 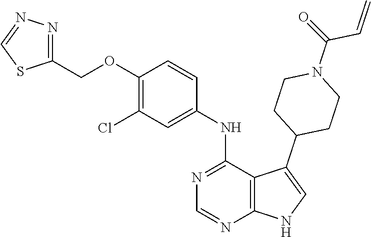 [SMILES: C=CC(=O)N1CCC(c2c[nH]c3ncnc(Nc4ccc(OCc5nncs5)c(Cl)c4)c23)CC1] | 1-(4-(4-((4-((1,3,4-thiadiazol-2-yl)methoxy)- 3-chlorophenyl)amino)-7H-pyrrolo[2,3- d]pyrimidin-5-yl)piperidin-1-yl)prop-2-en-1- one |
| 53 | 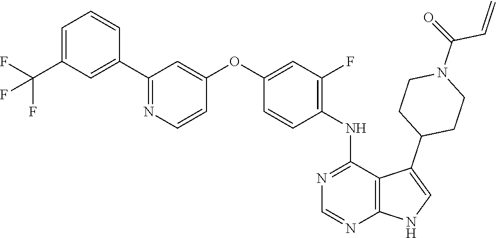 [SMILES: C=CC(=O)N1CCC(c2c[nH]c3ncnc(Nc4ccc(Oc5ccnc(-c6cccc(C(F)(F)F)c6)c5)cc4F)c23)CC1] | 1-(4-(4-((2-fluoro-4-((2-(3- (trifluoromethyl)phenyl)pyridin-4- yl)oxy)phenyl)amino)-7H-pyrrolo[2,3- d]pyrimidin-5-yl)piperidin-1-yl)prop-2-en-1- one |
| 54 | 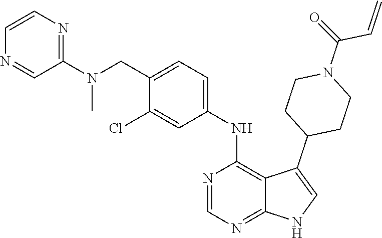 [SMILES: C=CC(=O)N1CCC(c2c[nH]c3ncnc(Nc4ccc(CN(C)c5cnccn5)c(Cl)c4)c23)CC1] | 1-(4-(4-((3-chloro-4-((methyl(pyrazin-2- yl)amino)methyl)phenyl)amino)-7H- pyrrolo[2,3-d]pyrimidin-5-yl)piperidin-1- yl)prop-2-en-1-one |
| 55 | 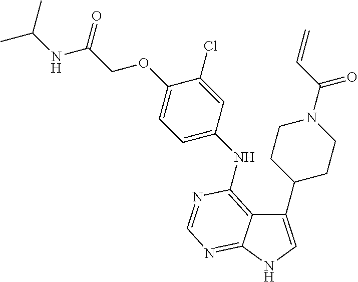 [SMILES: C=CC(=O)N1CCC(c2c[nH]c3ncnc(Nc4ccc(OCC(=O)NC(C)C)c(Cl)c4)c23)CC1] | 2-(4-((5-(1-acryloylpiperidin-4-yl)-7H- pyrrolo[2,3-d]pyrimidin-4-yl)amino)-2- chlorophenoxy)-N-isopropylacetamide |
| 56 | 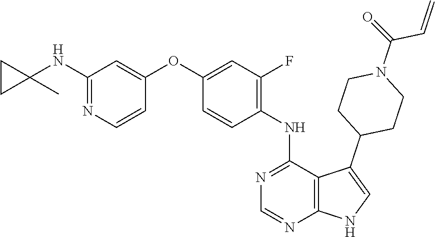 [SMILES: C=CC(=O)N1CCC(c2c[nH]c3ncnc(Nc4ccc(Oc5ccnc(NC6(C)CC6)c5)cc4F)c23)CC1] | 1-(4-(4-((2-fluoro-4-((2-((1- methylcyclopropyl)amino)pyridin-4- yl)oxy)phenyl)amino)-7H-pyrrolo[2,3- d]pyrimidin-5-yl)piperidin-1-yl)prop-2-en-1- one |
| 57 | 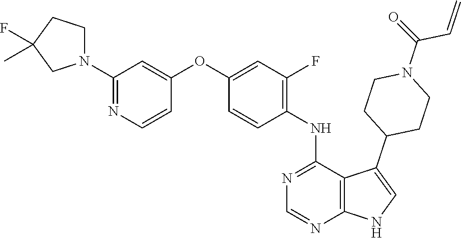 [SMILES: C=CC(=O)N1CCC(c2c[nH]c3ncnc(Nc4ccc(Oc5ccnc(N6CCC(C)(F)C6)c5)cc4F)c23)CC1] | 1-(4-(4-((2-fluoro-4-((2-(3-fluoro-3- methylpyrrolidin-1-yl)pyridin-4- yl)oxy)phenyl)amino)-7H-pyrrolo[2,3- d]pyrimidin-5-yl)piperidin-1-yl)prop-2-en-1- one |
| 58 | 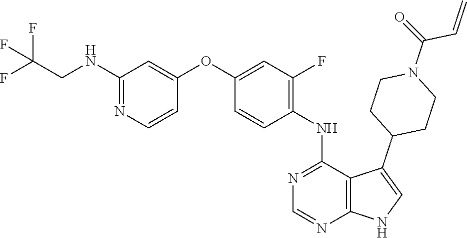 [SMILES: C=CC(=O)N1CCC(c2c[nH]c3ncnc(Nc4ccc(Oc5ccnc(NCC(F)(F)F)c5)cc4F)c23)CC1] | 1-(4-(4-((2-fluoro-4-((2-((2,2,2- trifluoroethyl)amino)pyridin-4- yl)oxy)phenyl)amino)-7H-pyrrolo[2,3- d]pyrimidin-5-yl)piperidin-1-yl)prop-2-en-1- one |
| 59 | 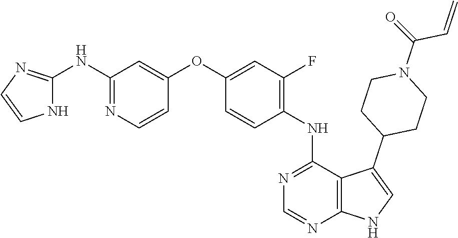 [SMILES: C=CC(=O)N1CCC(c2c[nH]c3ncnc(Nc4ccc(Oc5ccnc(Nc6ncc[nH]6)c5)cc4F)c23)CC1] | 1-(4-(4-((4-((2-((1H-imidazol-2- yl)amino)pyridin-4-yl)oxy)-2- fluorophenyl)amino)-7H-pyrrolo[2,3- d]pyrimidin-5-yl)piperidin-1-yl)prop-2-en-1- one |
| 60 | 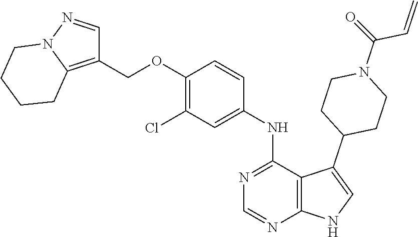 [SMILES: C=CC(=O)N1CCC(c2c[nH]c3ncnc(Nc4ccc(OCc5cnn6c5CCCC6)c(Cl)c4)c23)CC1] | 1-(4-(4-((3-chloro-4-((4,5,6,7- tetrahydropyrazolo[1,5-a]pyridin-3- yl)methoxy)phenyl)amino)-7H-pyrrolo[2,3- d]pyrimidin-5-yl)piperidin-1-yl)prop-2-en-1- one |
| 61 | 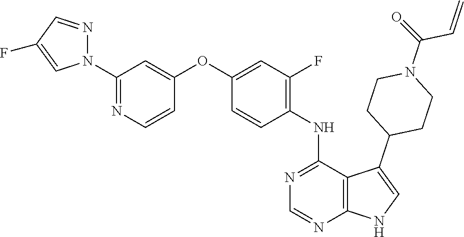 [SMILES: C=CC(=O)N1CCC(c2c[nH]c3ncnc(Nc4ccc(Oc5ccnc(-n6cc(F)cn6)c5)cc4F)c23)CC1] | 1-(4-(4-((2-fluoro-4-((2-(4-fluoro-1H-pyrazol- 1-yl)pyridin-4-yl)oxy)phenyl)amino)-7H- pyrrolo[2,3-d]pyrimidin-5-yl)piperidin-1- yl)prop-2-en-1-one |
| 62 | 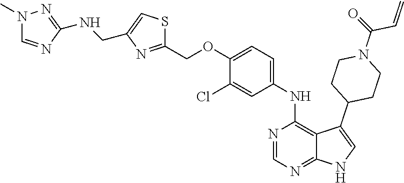 [SMILES: C=CC(=O)N1CCC(c2c[nH]c3ncnc(Nc4ccc(OCc5nc(CNc6ncn(C)n6)cs5)c(Cl)c4)c23)CC1] | 1-(4-(4-((3-chloro-4-((4-(((1-methyl-1H-1,2,4- triazol-3-yl)amino)methyl)thiazol-2- yl)methoxy)phenyl)amino)-7H-pyrrolo[2,3- d]pyrimidin-5-yl)piperidin-1-yl)prop-2-en-1- one |
| 63 | 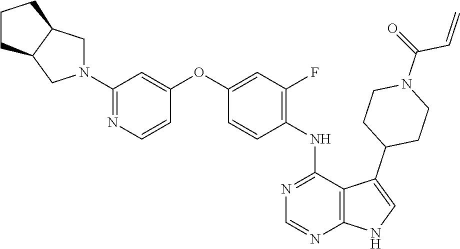 [SMILES: C=CC(=O)N1CCC(c2c[nH]c3ncnc(Nc4ccc(Oc5ccnc(N6C[C@H]7CCC[C@H]7C6)c5)cc4F)c23)CC1] | 1-(4-(4-((2-fluoro-4-((2-((3aR,6aS)- hexahydrocyclopenta[c]pyrrol-2(1H)- yl)pyridin-4-yl)oxy)phenyl)amino)-7H- pyrrolo[2,3-d]pyrimidin-5-yl)piperidin-1- yl)prop-2-en-1-one |
| 64 | 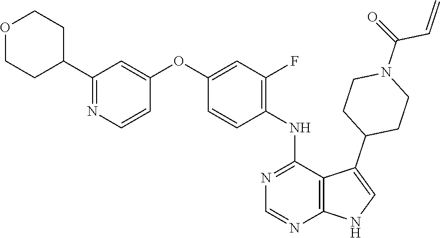 [SMILES: C=CC(=O)N1CCC(c2c[nH]c3ncnc(Nc4ccc(Oc5ccnc(C6CCOCC6)c5)cc4F)c23)CC1] | 1-(4-(4-((2-fluoro-4-((2-(tetrahydro-2H-pyran- 4-yl)pyridin-4-yl)oxy)phenyl)amino)-7H- pyrrolo[2,3-d]pyrimidin-5-yl)piperidin-1- yl)prop-2-en-1-one |
| 65 | 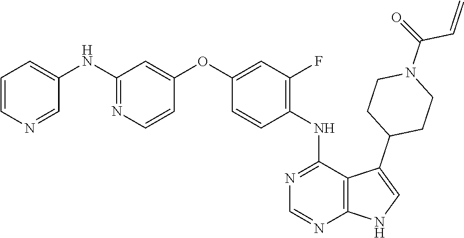 [SMILES: C=CC(=O)N1CCC(c2c[nH]c3ncnc(Nc4ccc(Oc5ccnc(Nc6cccnc6)c5)cc4F)c23)CC1] | 1-(4-(4-((2-fluoro-4-((2-(pyridin-3- ylamino)pyridin-4-yl)oxy)phenyl)amino)-7H- pyrrolo[2,3-d]pyrimidin-5-yl)piperidin-1- yl)prop-2-en-1-one |
| 66 | 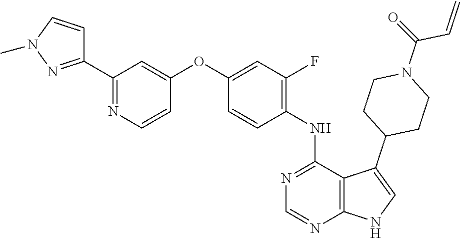 [SMILES: C=CC(=O)N1CCC(c2c[nH]c3ncnc(Nc4ccc(Oc5ccnc(-c6ccn(C)n6)c5)cc4F)c23)CC1] | 1-(4-(4-((2-fluoro-4-((2-(1-methyl-1H- pyrazol-3-yl)pyridin-4-yl)oxy)phenyl)amino)- 7H-pyrrolo[2,3-d]pyrimidin-5-yl)piperidin-1- yl)prop-2-en-1-one |
| 67 | 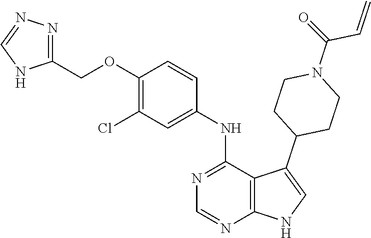 [SMILES: C=CC(=O)N1CCC(c2c[nH]c3ncnc(Nc4ccc(OCc5nnc[nH]5)c(Cl)c4)c23)CC1] | 1-(4-(4-((4-((4H-1,2,4-triazol-3-yl)methoxy)- 3-chlorophenyl)amino)-7H-pyrrolo[2,3- d]pyrimidin-5-yl)piperidin-1-yl)prop-2-en-1- one |
| 68 | 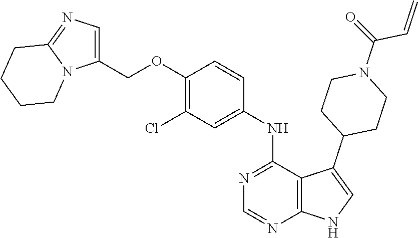 [SMILES: C=CC(=O)N1CCC(c2c[nH]c3ncnc(Nc4ccc(OCc5cnc6n5CCCC6)c(Cl)c4)c23)CC1] | 1-(4-(4-((3-chloro-4-((5,6,7,8- tetrahydroimidazo[1,2-a]pyridin-3- yl)methoxy)phenyl)amino)-7H-pyrrolo[2,3- d]pyrimidin-5-yl)piperidin-1-yl)prop-2-en-1- one |
| 69 | 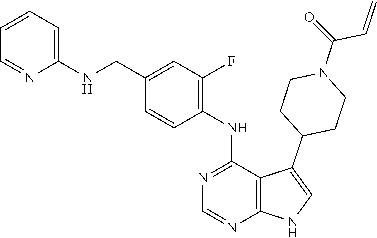 [SMILES: C=CC(=O)N1CCC(c2c[nH]c3ncnc(Nc4ccc(CNc5ccccn5)cc4F)c23)CC1] | 1-(4-(4-((2-fluoro-4-((pyridin-2- ylamino)methyl)phenyl)amino)-7H- pyrrolo[2,3-d]pyrimidin-5-yl)piperidin-1- yl)prop-2-en-1-one |
| 70 | 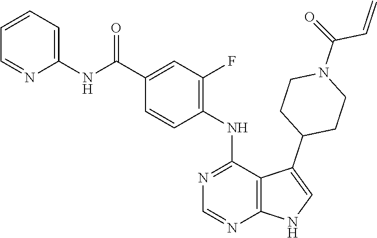 [SMILES: C=CC(=O)N1CCC(c2c[nH]c3ncnc(Nc4ccc(C(=O)Nc5ccccn5)cc4F)c23)CC1] | 4-((5-(1-acryloylpiperidin-4-yl)-7H- pyrrolo[2,3-d]pyrimidin-4-yl)amino)-3-fluoro- N-(pyridin-2-yl)benzamide |
| 71 | 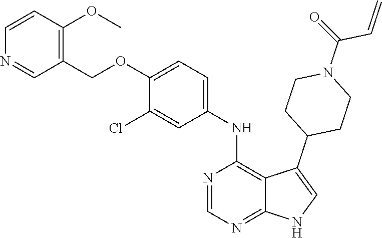 [SMILES: C=CC(=O)N1CCC(c2c[nH]c3ncnc(Nc4ccc(OCc5cnccc5OC)c(Cl)c4)c23)CC1] | 1-(4-(4-((3-chloro-4-((4-methoxypyridin-3- yl)methoxy)phenyl)amino)-7H-pyrrolo[2,3- d]pyrimidin-5-yl)piperidin-1-yl)prop-2-en-1- one |
| 72 | 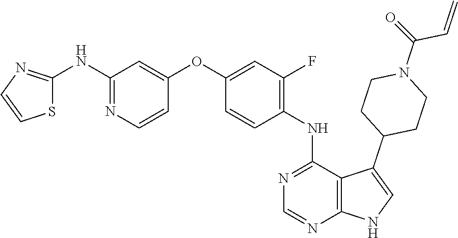 [SMILES: C=CC(=O)N1CCC(c2c[nH]c3ncnc(Nc4ccc(Oc5ccnc(Nc6nccs6)c5)cc4F)c23)CC1] | 1-(4-(4-((2-fluoro-4-((2-(thiazol-2- ylamino)pyridin-4-yl)oxy)phenyl)amino)-7H- pyrrolo[2,3-d]pyrimidin-5-yl)piperidin-1- yl)prop-2-en-1-one |
| 73 | 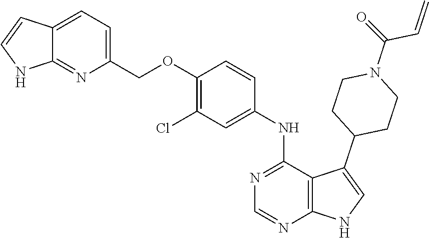 [SMILES: C=CC(=O)N1CCC(c2c[nH]c3ncnc(Nc4ccc(OCc5ccc6cc[nH]c6n5)c(Cl)c4)c23)CC1] | 1-(4-(4-((4-((1H-pyrrolo[2,3-b]pyridin-6- yl)methoxy)-3-chlorophenyl)amino)-7H- pyrrolo[2,3-d]pyrimidin-5-yl)piperidin-1- yl)prop-2-en-1-one |
| 74 | 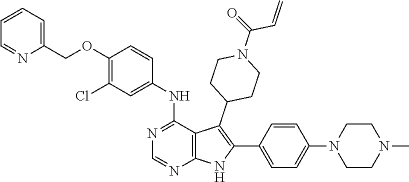 [SMILES: C=CC(=O)N1CCC(c2c(-c3ccc(N4CCN(C)CC4)cc3)[nH]c3ncnc(Nc4ccc(OCc5ccccn5)c(Cl)c4)c23)CC1] | 1-(4-(4-((3-chloro-4-(pyridin-2- ylmethoxy)phenyl)amino)-6-(4-(4- methylpiperazin-1-yl)phenyl)-7H-pyrrolo[2,3- d]pyrimidin-5-yl)piperidin-1-yl)prop-2-en-1- one |
| 75 | 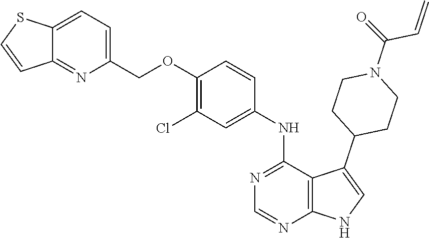 [SMILES: C=CC(=O)N1CCC(c2c[nH]c3ncnc(Nc4ccc(OCc5ccc6sccc6n5)c(Cl)c4)c23)CC1] | 1-(4-(4-((3-chloro-4-(thieno[3,2-b]pyridin-5- ylmethoxy)phenyl)amino)-7H-pyrrolo[2,3- d]pyrimidin-5-yl)piperidin-1-yl)prop-2-en-1- one |
| 76 | 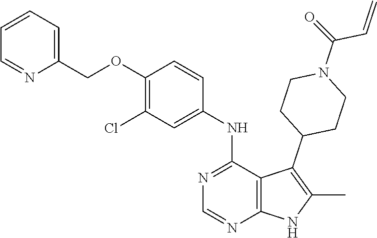 [SMILES: C=CC(=O)N1CCC(c2c(C)[nH]c3ncnc(Nc4ccc(OCc5ccccn5)c(Cl)c4)c23)CC1] | 1-(4-(4-((3-chloro-4-(pyridin-2- ylmethoxy)phenyl)amino)-6-methyl-7H- pyrrolo[2,3-d]pyrimidin-5-yl)piperidin-1- yl)prop-2-en-1-one |
| 77 | 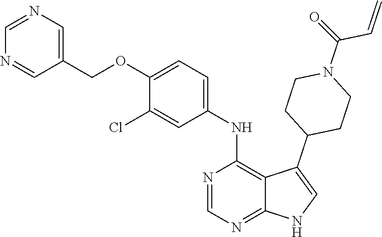 [SMILES: C=CC(=O)N1CCC(c2c[nH]c3ncnc(Nc4ccc(OCc5cncnc5)c(Cl)c4)c23)CC1] | 1-(4-(4-((3-chloro-4-(pyrimidin-5- ylmethoxy)phenyl)amino)-7H-pyrrolo[2,3- d]pyrimidin-5-yl)piperidin-1-yl)prop-2-en-1- one |
| 78 | 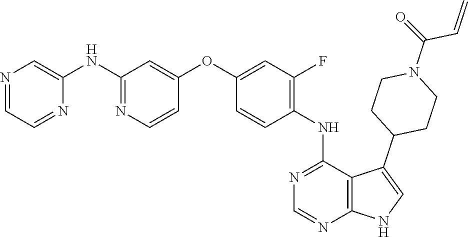 [SMILES: C=CC(=O)N1CCC(c2c[nH]c3ncnc(Nc4ccc(Oc5ccnc(Nc6cnccn6)c5)cc4F)c23)CC1] | 1-(4-(4-((2-fluoro-4-((2-(pyrazin-2- ylamino)pyridin-4-yl)oxy)phenyl)amino)-7H- pyrrolo[2,3-d]pyrimidin-5-yl)piperidin-1- yl)prop-2-en-1-one |
| 79 | 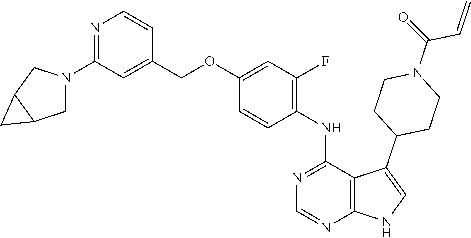 [SMILES: C=CC(=O)N1CCC(c2c[nH]c3ncnc(Nc4ccc(OCc5ccnc(N6CC7CC7C6)c5)cc4F)c23)CC1] | 1-(4-(4-((4-((2-(3-azabicyclo[3.1.0]hexan-3- yl)pyridin-4-yl)methoxy)-2- fluorophenyl)amino)-7H-pyrrolo[2,3- d]pyrimidin-5-yl)piperidin-1-yl)prop-2-en-1- one |
| 80 | 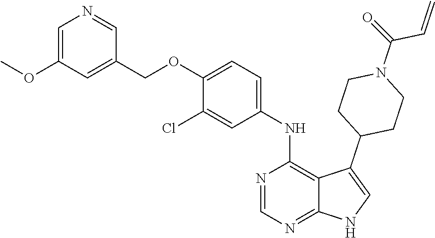 [SMILES: C=CC(=O)N1CCC(c2c[nH]c3ncnc(Nc4ccc(OCc5cncc(OC)c5)c(Cl)c4)c23)CC1] | 1-(4-(4-((3-chloro-4-((5-methoxypyridin-3- yl)methoxy)phenyl)amino)-7H-pyrrolo[2,3- d]pyrimidin-5-yl)piperidin-1-yl)prop-2-en-1- one |
| 81 | 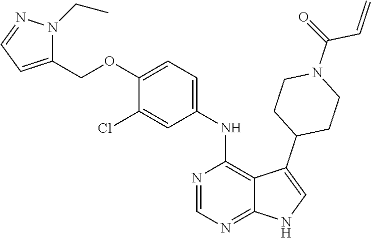 [SMILES: C=CC(=O)N1CCC(c2c[nH]c3ncnc(Nc4ccc(OCc5ccnn5CC)c(Cl)c4)c23)CC1] | 1-(4-(4-((3-chloro-4-((1-ethyl-1H-pyrazol-5- yl)methoxy)phenyl)amino)-7H-pyrrolo[2,3- d]pyrimidin-5-yl)piperidin-1-yl)prop-2-en-1- one |
| 82 | 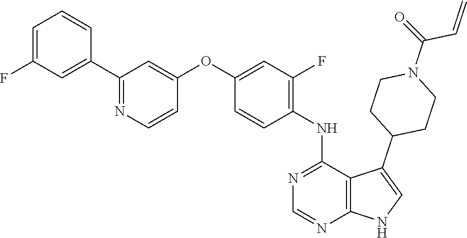 [SMILES: C=CC(=O)N1CCC(c2c[nH]c3ncnc(Nc4ccc(Oc5ccnc(-c6cccc(F)c6)c5)cc4F)c23)CC1] | 1-(4-(4-((2-fluoro-4-((2-(3- fluorophenyl)pyridin-4-yl)oxy)phenyl)amino)- 7H-pyrrolo[2,3-d]pyrimidin-5-yl)piperidin-1- yl)prop-2-en-1-one |
| 83 | 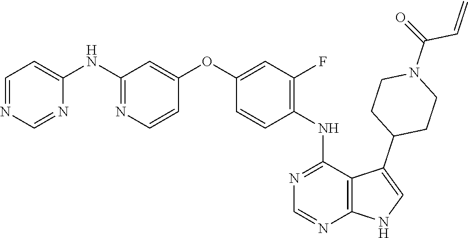 [SMILES: C=CC(=O)N1CCC(c2c[nH]c3ncnc(Nc4ccc(Oc5ccnc(Nc6ccncn6)c5)cc4F)c23)CC1] | 1-(4-(4-((2-fluoro-4-((2-(pyrimidin-4- ylamino)pyridin-4-yl)oxy)phenyl)amino)-7H- pyrrolo[2,3-d]pyrimidin-5-yl)piperidin-1- yl)prop-2-en-1-one |
| 84 | 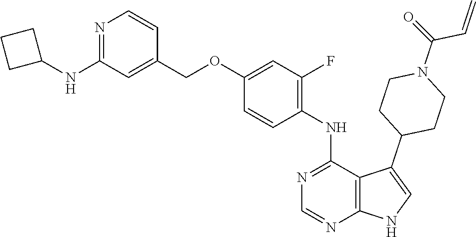 [SMILES: C=CC(=O)N1CCC(c2c[nH]c3ncnc(Nc4ccc(OCc5ccnc(NC6CCC6)c5)cc4F)c23)CC1] | 1-(4-(4-((4-((2-(cycloButylamino)pyridin-4- yl)methoxy)-2-fluorophenyl)amino)-7H- pyrrolo[2,3-d]pyrimidin-5-yl)piperidin-1- yl)prop-2-en-1-one |
| 85 | 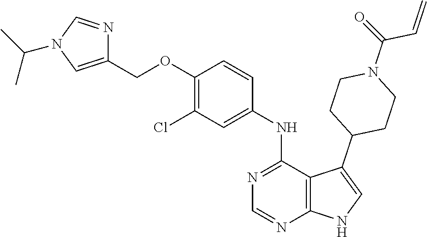 [SMILES: C=CC(=O)N1CCC(c2c[nH]c3ncnc(Nc4ccc(OCc5cn(C(C)C)cn5)c(Cl)c4)c23)CC1] | 1-(4-(4-((3-chloro-4-((1-isopropyl-1H- imidazol-4-yl)methoxy)phenyl)amino)-7H- pyrrolo[2,3-d]pyrimidin-5-yl)piperidin-1- yl)prop-2-en-1-one |
| 86 | 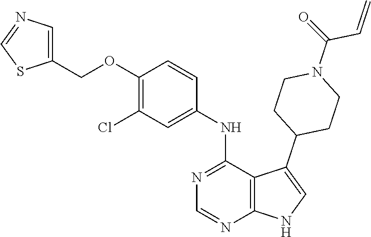 [SMILES: C=CC(=O)N1CCC(c2c[nH]c3ncnc(Nc4ccc(OCc5cncs5)c(Cl)c4)c23)CC1] | 1-(4-(4-((3-chloro-4-(thiazol-5- ylmethoxy)phenyl)amino)-7H-pyrrolo[2,3- d]pyrimidin-5-yl)piperidin-1-yl)prop-2-en-1- one |
| 87 | 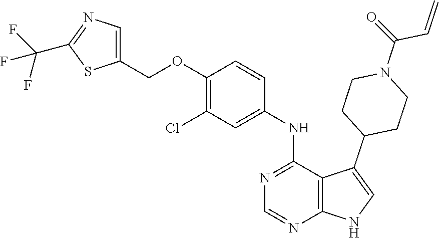 [SMILES: C=CC(=O)N1CCC(c2c[nH]c3ncnc(Nc4ccc(OCc5cnc(C(F)(F)F)s5)c(Cl)c4)c23)CC1] | 1-(4-(4-((3-chloro-4-((2- (trifluoromethyl)thiazol-5- yl)methoxy)phenyl)amino)-7H-pyrrolo[2,3- d]pyrimidin-5-yl)piperidin-1-yl)prop-2-en-1- one |
| 88 | 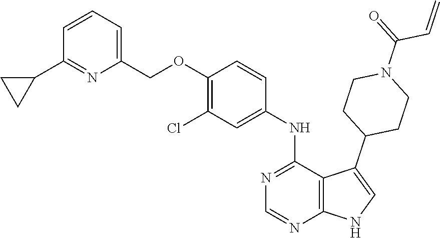 [SMILES: C=CC(=O)N1CCC(c2c[nH]c3ncnc(Nc4ccc(OCc5cccc(C6CC6)n5)c(Cl)c4)c23)CC1] | 1-(4-(4-((3-chloro-4-((6-cyclopropylpyridin-2- yl)methoxy)phenyl)amino)-7H-pyrrolo[2,3- d]pyrimidin-5-yl)piperidin-1-yl)prop-2-en-1- one |
| 89 | 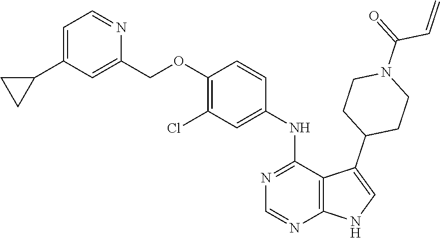 [SMILES: C=CC(=O)N1CCC(c2c[nH]c3ncnc(Nc4ccc(OCc5cc(C6CC6)ccn5)c(Cl)c4)c23)CC1] | 1-(4-(4-((3-chloro-4-((4-cyclopropylpyridin-2- yl)methoxy)phenyl)amino)-7H-pyrrolo[2,3- d]pyrimidin-5-yl)piperidin-1-yl)prop-2-en-1- one |
| 90 | 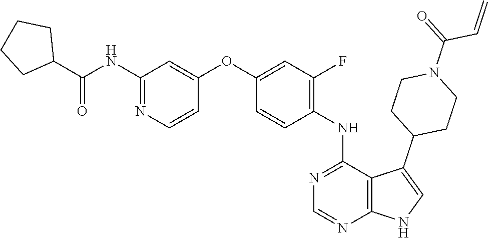 [SMILES: C=CC(=O)N1CCC(c2c[nH]c3ncnc(Nc4ccc(Oc5ccnc(NC(=O)C6CCCC6)c5)cc4F)c23)CC1] | N-(4-(4-((5-(1-acryloylpiperidin-4-yl)-7H- pyrrolo[2,3-d]pyrimidin-4-yl)amino)-3- fluorophenoxy)pyridin-2- yl)cyclopentanecarboxamide |
| 91 | 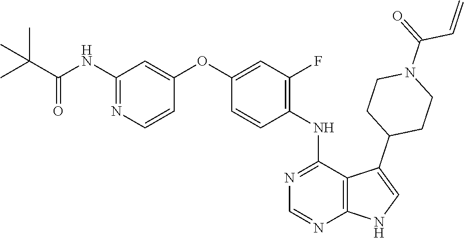 [SMILES: C=CC(=O)N1CCC(c2c[nH]c3ncnc(Nc4ccc(Oc5ccnc(NC(=O)C(C)(C)C)c5)cc4F)c23)CC1] | N-(4-(4-((5-(1-acryloylpiperidin-4-yl)-7H- pyrrolo[2,3-d]pyrimidin-4-yl)amino)-3- fluorophenoxy)pyridin-2-yl)pivalamide |
| 92 | 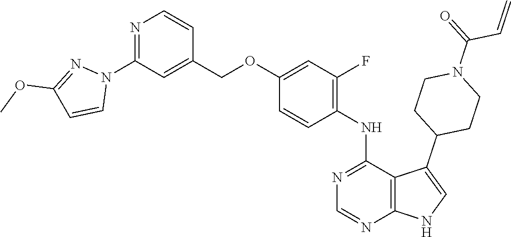 [SMILES: C=CC(=O)N1CCC(c2c[nH]c3ncnc(Nc4ccc(OCc5ccnc(-n6ccc(OC)n6)c5)cc4F)c23)CC1] | 1-(4-(4-((2-fluoro-4-((2-(3-methoxy-1H- pyrazol-1-yl)pyridin-4- yl)methoxy)phenyl)amino)-7H-pyrrolo[2,3- d]pyrimidin-5-yl)piperidin-1-yl)prop-2-en-1- one |
| 93 | 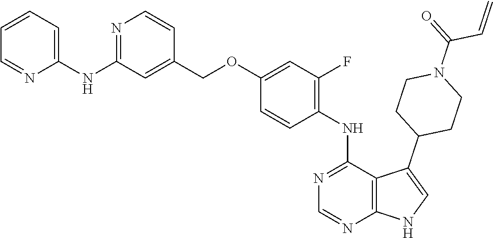 [SMILES: C=CC(=O)N1CCC(c2c[nH]c3ncnc(Nc4ccc(OCc5ccnc(Nc6ccccn6)c5)cc4F)c23)CC1] | 1-(4-(4-((2-fluoro-4-((2-(pyridin-2- ylamino)pyridin-4-yl)methoxy)phenyl)amino)- 7H-pyrrolo[2,3-d]pyrimidin-5-yl)piperidin-1- yl)prop-2-en-1-one |
| 94 | 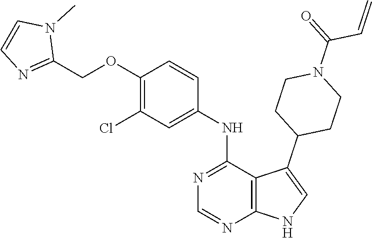 [SMILES: C=CC(=O)N1CCC(c2c[nH]c3ncnc(Nc4ccc(OCc5nccn5C)c(Cl)c4)c23)CC1] | 1-(4-(4-((3-chloro-4-((1-methyl-1H-imidazol- 2-yl)methoxy)phenyl)amino)-7H-pyrrolo[2,3- d]pyrimidin-5-yl)piperidin-1-yl)prop-2-en-1- one |
| 95 | 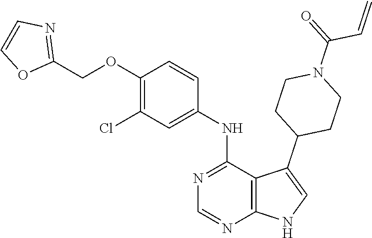 [SMILES: C=CC(=O)N1CCC(c2c[nH]c3ncnc(Nc4ccc(OCc5ncco5)c(Cl)c4)c23)CC1] | 1-(4-(4-((3-chloro-4-(oxazol-2- ylmethoxy)phenyl)amino)-7H-pyrrolo[2,3- d]pyrimidin-5-yl)piperidin-1-yl)prop-2-en-1- one |
| 96 | 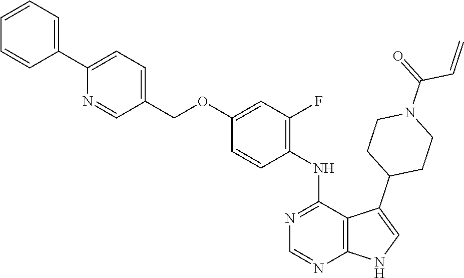 [SMILES: C=CC(=O)N1CCC(c2c[nH]c3ncnc(Nc4ccc(OCc5ccc(-c6ccccc6)nc5)cc4F)c23)CC1] | 1-(4-(4-((2-fluoro-4-((6-phenylpyridin-3- yl)methoxy)phenyl)amino)-7H-pyrrolo[2,3- d]pyrimidin-5-yl)piperidin-1-yl)prop-2-en-1- one |
| 97 | 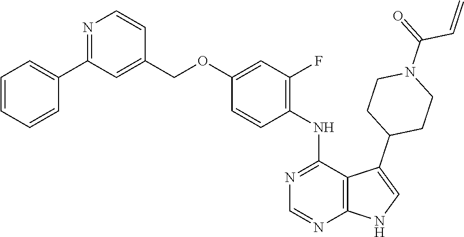 [SMILES: C=CC(=O)N1CCC(c2c[nH]c3ncnc(Nc4ccc(OCc5ccnc(-c6ccccc6)c5)cc4F)c23)CC1] | 1-(4-(4-((2-fluoro-4-((2-phenylpyridin-4- yl)methoxy)phenyl)amino)-7H-pyrrolo[2,3- d]pyrimidin-5-yl)piperidin-1-yl)prop-2-en-1- one |
| 98 | 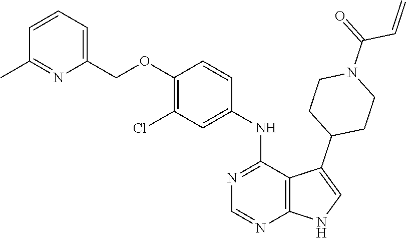 [SMILES: C=CC(=O)N1CCC(c2c[nH]c3ncnc(Nc4ccc(OCc5cccc(C)n5)c(Cl)c4)c23)CC1] | 1-(4-(4-((3-chloro-4-((6-methylpyridin-2- yl)methoxy)phenyl)amino)-7H-pyrrolo[2,3- d]pyrimidin-5-yl)piperidin-1-yl)prop-2-en-1- one |
| 99 | 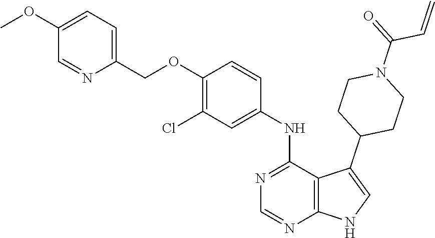 [SMILES: C=CC(=O)N1CCC(c2c[nH]c3ncnc(Nc4ccc(OCc5ccc(OC)cn5)c(Cl)c4)c23)CC1] | 1-(4-(4-((3-chloro-4-((5-methoxypyridin-2- yl)methoxy)phenyl)amino)-7H-pyrrolo[2,3- d]pyrimidin-5-yl)piperidin-1-yl)prop-2-en-1- one |
| 100 | 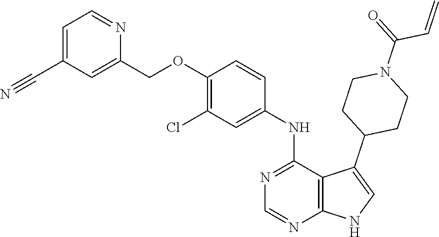 [SMILES: C=CC(=O)N1CCC(c2c[nH]c3ncnc(Nc4ccc(OCc5cc(C#N)ccn5)c(Cl)c4)c23)CC1] | 2-((4-((5-(1-acryloylpiperidin-4-yl)-7H- pyrrolo[2,3-d]pyrimidin-4-yl)amino)-2- chlorophenoxy)methyl)isonicotinonitrile |
| 101 | 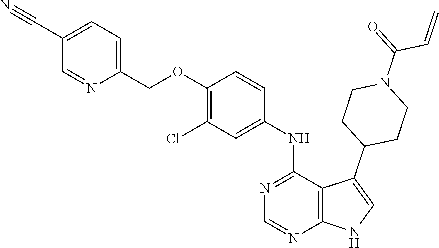 [SMILES: C=CC(=O)N1CCC(c2c[nH]c3ncnc(Nc4ccc(OCc5ccc(C#N)cn5)c(Cl)c4)c23)CC1] | 6-((4-((5-(1-acryloylpiperidin-4-yl)-7H- pyrrolo[2,3-d]pyrimidin-4-yl)amino)-2- chlorophenoxy)methyl)nicotinonitrile |
| 102 | 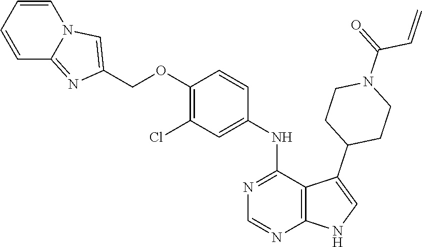 [SMILES: C=CC(=O)N1CCC(c2c[nH]c3ncnc(Nc4ccc(OCc5cn6ccccc6n5)c(Cl)c4)c23)CC1] | 1-(4-(4-((3-chloro-4-(imidazo[1,2-a]pyridin-2- ylmethoxy)phenyl)amino)-7H-pyrrolo[2,3- d]pyrimidin-5-yl)piperidin-1-yl)prop-2-en-1- one |
| 103 | 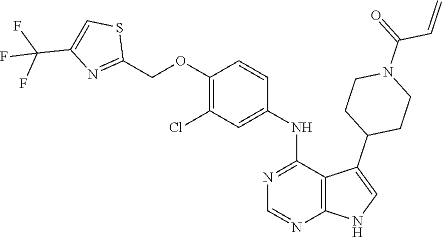 [SMILES: C=CC(=O)N1CCC(c2c[nH]c3ncnc(Nc4ccc(OCc5nc(C(F)(F)F)cs5)c(Cl)c4)c23)CC1] | 1-(4-(4-((3-chloro-4-((4- (trifluoromethyl)thiazol-2- yl)methoxy)phenyl)amino)-7H-pyrrolo[2,3- d]pyrimidin-5-yl)piperidin-1-yl)prop-2-en-1- one |
| 104 | 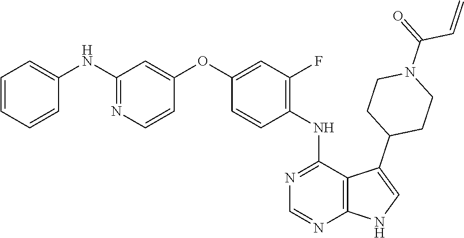 [SMILES: C=CC(=O)N1CCC(c2c[nH]c3ncnc(Nc4ccc(Oc5ccnc(Nc6ccccc6)c5)cc4F)c23)CC1] | 1-(4-(4-((2-fluoro-4-((2-(phenylamino)pyridin- 4-yl)oxy)phenyl)amino)-7H-pyrrolo[2,3- d]pyrimidin-5-yl)piperidin-1-yl)prop-2-en-1- one |
| 105 | 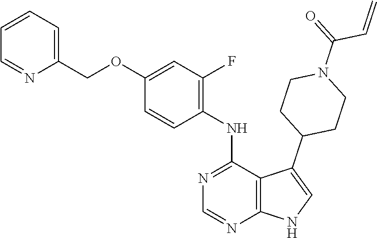 [SMILES: C=CC(=O)N1CCC(c2c[nH]c3ncnc(Nc4ccc(OCc5ccccn5)cc4F)c23)CC1] | 1-(4-(4-((2-fluoro-4-(pyridin-2- ylmethoxy)phenyl)amino)-7H-pyrrolo[2,3- d]pyrimidin-5-yl)piperidin-1-yl)prop-2-en-1- one |
| 106 | 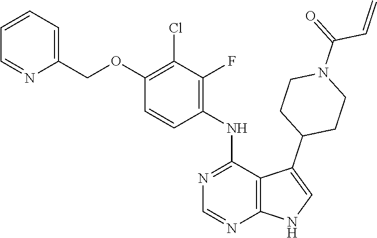 [SMILES: C=CC(=O)N1CCC(c2c[nH]c3ncnc(Nc4ccc(OCc5ccccn5)c(Cl)c4F)c23)CC1] | 1-(4-(4-((3-chloro-2-fluoro-4-(pyridin-2- ylmethoxy)phenyl)amino)-7H-pyrrolo[2,3- d]pyrimidin-5-yl)piperidin-1-yl)prop-2-en-1- one |
| 107 | 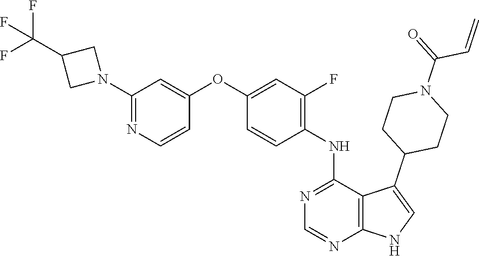 [SMILES: C=CC(=O)N1CCC(c2c[nH]c3ncnc(Nc4ccc(Oc5ccnc(N6CC(C(F)(F)F)C6)c5)cc4F)c23)CC1] | 1-(4-(4-((2-fluoro-4-((2-(3- (trifluoromethyl)azetidin-1-yl)pyridin-4- yl)oxy)phenyl)amino)-7H-pyrrolo[2,3- d]pyrimidin-5-yl)piperidin-1-yl)prop-2-en-1- one |
| 108 | 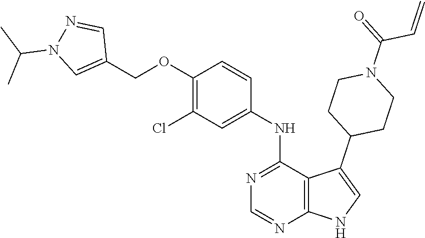 [SMILES: C=CC(=O)N1CCC(c2c[nH]c3ncnc(Nc4ccc(OCc5cnn(C(C)C)c5)c(Cl)c4)c23)CC1] | 1-(4-(4-((3-chloro-4-((1-isopropyl-1H- pyrazol-4-yl)methoxy)phenyl)amino)-7H- pyrrolo[2,3-d]pyrimidin-5-yl)piperidin-1- yl)prop-2-en-1-one |
| 109 | 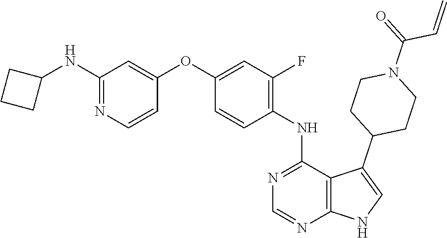 [SMILES: C=CC(=O)N1CCC(c2c[nH]c3ncnc(Nc4ccc(Oc5ccnc(NC6CCC6)c5)cc4F)c23)CC1] | 1-(4-(4-((4-((2-(cycloButylamino)pyridin-4- yl)oxy)-2-fluorophenyl)amino)-7H- pyrrolo[2,3-d]pyrimidin-5-yl)piperidin-1- yl)prop-2-en-1-one |
| 110 | 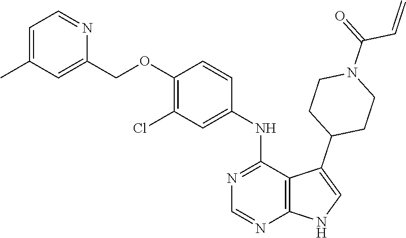 [SMILES: C=CC(=O)N1CCC(c2c[nH]c3ncnc(Nc4ccc(OCc5cc(C)ccn5)c(Cl)c4)c23)CC1] | 1-(4-(4-((3-chloro-4-((4-methylpyridin-2- yl)methoxy)phenyl)amino)-7H-pyrrolo[2,3- d]pyrimidin-5-yl)piperidin-1-yl)prop-2-en-1- one |
| 111 | 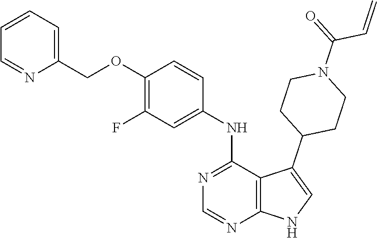 [SMILES: C=CC(=O)N1CCC(c2c[nH]c3ncnc(Nc4ccc(OCc5ccccn5)c(F)c4)c23)CC1] | 1-(4-(4-((3-fluoro-4-(pyridin-2- ylmethoxy)phenyl)amino)-7H-pyrrolo[2,3- d]pyrimidin-5-yl)piperidin-1-yl)prop-2-en-1- one |
| 112 | 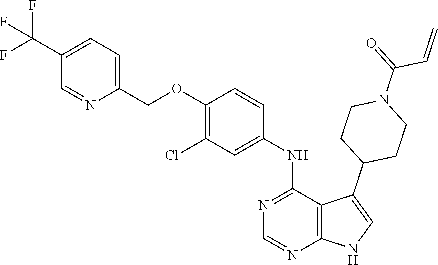 [SMILES: C=CC(=O)N1CCC(c2c[nH]c3ncnc(Nc4ccc(OCc5ccc(C(F)(F)F)cn5)c(Cl)c4)c23)CC1] | 1-(4-(4-((3-chloro-4-((5- (trifluoromethyl)pyridin-2- yl)methoxy)phenyl)amino)-7H-pyrrolo[2,3- d]pyrimidin-5-yl)piperidin-1-yl)prop-2-en-1- one |
| 113 | 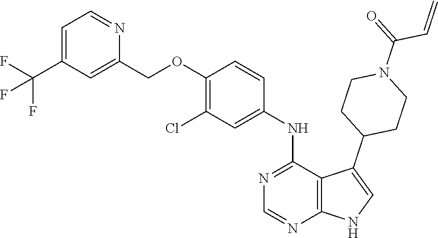 [SMILES: C=CC(=O)N1CCC(c2c[nH]c3ncnc(Nc4ccc(OCc5cc(C(F)(F)F)ccn5)c(Cl)c4)c23)CC1] | 1-(4-(4-((3-chloro-4-((4- (trifluoromethyl)pyridin-2- yl)methoxy)phenyl)amino)-7H-pyrrolo[2,3- d]pyrimidin-5-yl)piperidin-1-yl)prop-2-en-1- one |
| 114 | 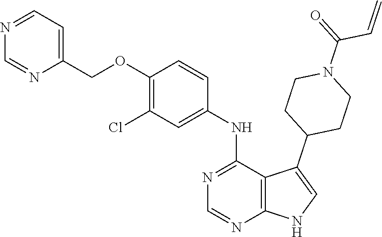 [SMILES: C=CC(=O)N1CCC(c2c[nH]c3ncnc(Nc4ccc(OCc5ccncn5)c(Cl)c4)c23)CC1] | 1-(4-(4-((3-chloro-4-(pyrimidin-4- ylmethoxy)phenyl)amino)-7H-pyrrolo[2,3- d]pyrimidin-5-yl)piperidin-1-yl)prop-2-en-1- one |
| 115 | 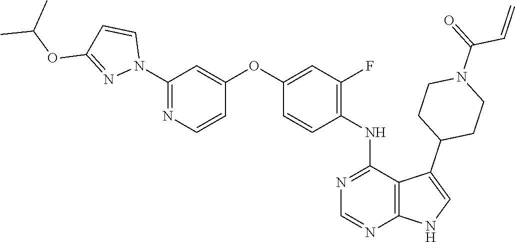 [SMILES: C=CC(=O)N1CCC(c2c[nH]c3ncnc(Nc4ccc(Oc5ccnc(-n6ccc(OC(C)C)n6)c5)cc4F)c23)CC1] | 1-(4-(4-((2-fluoro-4-((2-(3-isopropoxy-1H- pyrazol-1-yl)pyridin-4-yl)oxy)phenyl)amino)- 7H-pyrrolo[2,3-d]pyrimidin-5-yl)piperidin-1- yl)prop-2-en-1-one |
| 116 | 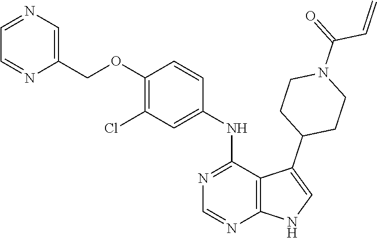 [SMILES: C=CC(=O)N1CCC(c2c[nH]c3ncnc(Nc4ccc(OCc5cnccn5)c(Cl)c4)c23)CC1] | 1-(4-(4-((3-chloro-4-(pyrazin-2- ylmethoxy)phenyl)amino)-7H-pyrrolo[2,3- d]pyrimidin-5-yl)piperidin-1-yl)prop-2-en-1- one |
| 117 | 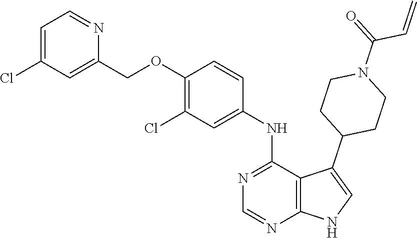 [SMILES: C=CC(=O)N1CCC(c2c[nH]c3ncnc(Nc4ccc(OCc5cc(Cl)ccn5)c(Cl)c4)c23)CC1] | 1-(4-(4-((3-chloro-4-((4-chloropyridin-2- yl)methoxy)phenyl)amino)-7H-pyrrolo[2,3- d]pyrimidin-5-yl)piperidin-1-yl)prop-2-en-1- one |
| 118 | 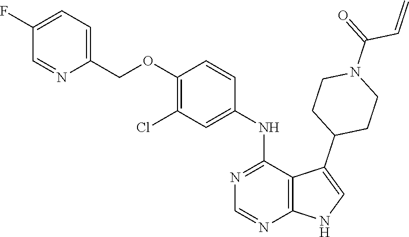 [SMILES: C=CC(=O)N1CCC(c2c[nH]c3ncnc(Nc4ccc(OCc5ccc(F)cn5)c(Cl)c4)c23)CC1] | 1-(4-(4-((3-chloro-4-((5-fluoropyridin-2- yl)methoxy)phenyl)amino)-7H-pyrrolo[2,3- d]pyrimidin-5-yl)piperidin-1-yl)prop-2-en-1- one |
| 119 | 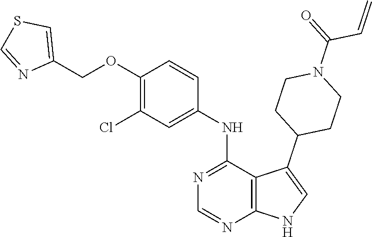 [SMILES: C=CC(=O)N1CCC(c2c[nH]c3ncnc(Nc4ccc(OCc5cscn5)c(Cl)c4)c23)CC1] | 1-(4-(4-((3-chloro-4-(thiazol-4- ylmethoxy)phenyl)amino)-7H-pyrrolo[2,3- d]pyrimidin-5-yl)piperidin-1-yl)prop-2-en-1- one |
| 120 | 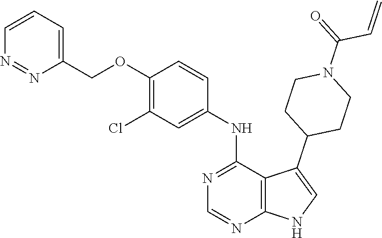 [SMILES: C=CC(=O)N1CCC(c2c[nH]c3ncnc(Nc4ccc(OCc5cccnn5)c(Cl)c4)c23)CC1] | 1-(4-(4-((3-chloro-4-(pyridazin-3- ylmethoxy)phenyl)amino)-7H-pyrrolo[2,3- d]pyrimidin-5-yl)piperidin-1-yl)prop-2-en-1- one |
| 121 | 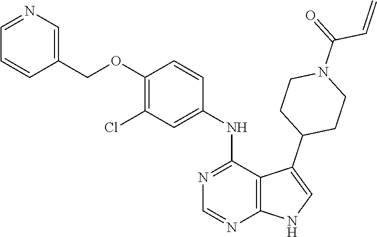 [SMILES: C=CC(=O)N1CCC(c2c[nH]c3ncnc(Nc4ccc(OCc5cccnc5)c(Cl)c4)c23)CC1] | 1-(4-(4-((3-chloro-4-(pyridin-3- ylmethoxy)phenyl)amino)-7H-pyrrolo[2,3- d]pyrimidin-5-yl)piperidin-1-yl)prop-2-en-1- one |
| 122 | 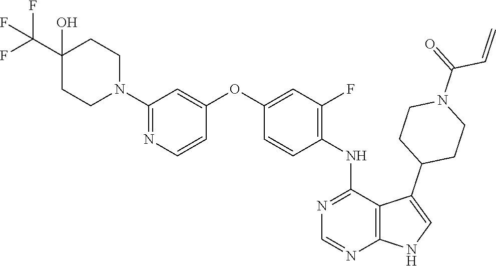 [SMILES: C=CC(=O)N1CCC(c2c[nH]c3ncnc(Nc4ccc(Oc5ccnc(N6CCC(O)(C(F)(F)F)CC6)c5)cc4F)c23)CC1] | 1-(4-(4-((2-fluoro-4-((2-(4-hydroxy-4- (trifluoromethyl)piperidin-1-yl)pyridin-4- yl)oxy)phenyl)amino)-7H-pyrrolo[2,3- d]pyrimidin-5-yl)piperidin-1-yl)prop-2-en-1- one |
| 123 | 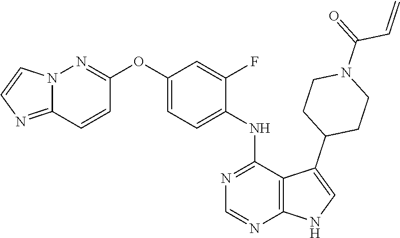 [SMILES: C=CC(=O)N1CCC(c2c[nH]c3ncnc(Nc4ccc(Oc5ccc6nccn6n5)cc4F)c23)CC1] | 1-(4-(4-((2-fluoro-4-(imidazo[1,2-b]pyridazin- 6-yloxy)phenyl)amino)-7H-pyrrolo[2,3- d]pyrimidin-5-yl)piperidin-1-yl)prop-2-en-1- one |
| 124 | 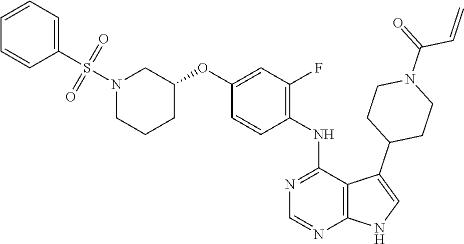 [SMILES: C=CC(=O)N1CCC(c2c[nH]c3ncnc(Nc4ccc(O[C@@H]5CCCN(S(=O)(=O)c6ccccc6)C5)cc4F)c23)CC1] | (S)-1-(4-(4-((2-fluoro-4-((1- (phenylsulfonyl)piperidin-3- yl)oxy)phenyl)amino)-7H-pyrrolo[2,3- d]pyrimidin-5-yl)piperidin-1-yl)prop-2-en-1- one |
| 125 | 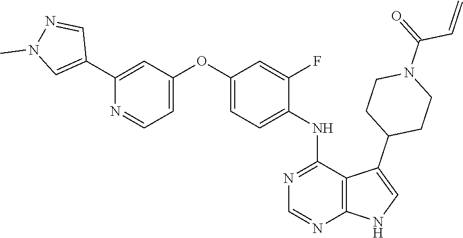 [SMILES: C=CC(=O)N1CCC(c2c[nH]c3ncnc(Nc4ccc(Oc5ccnc(-c6cnn(C)c6)c5)cc4F)c23)CC1] | 1-(4-(4-((2-fluoro-4-((2-(1-methyl-1H- pyrazol-4-yl)pyridin-4-yl)oxy)phenyl)amino)- 7H-pyrrolo[2,3-d]pyrimidin-5-yl)piperidin-1- yl)prop-2-en-1-one |
| 126 | 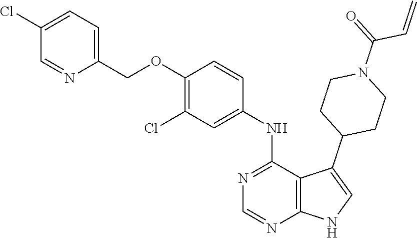 [SMILES: C=CC(=O)N1CCC(c2c[nH]c3ncnc(Nc4ccc(OCc5ccc(Cl)cn5)c(Cl)c4)c23)CC1] | 1-(4-(4-((3-chloro-4-((5-chloropyridin-2- yl)methoxy)phenyl)amino)-7H-pyrrolo[2,3- d]pyrimidin-5-yl)piperidin-1-yl)prop-2-en-1- one |
| 127 | 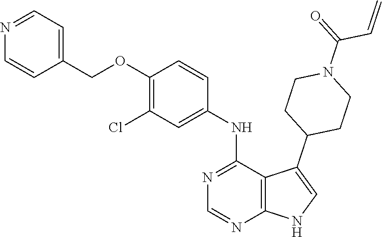 [SMILES: C=CC(=O)N1CCC(c2c[nH]c3ncnc(Nc4ccc(OCc5ccncc5)c(Cl)c4)c23)CC1] | 1-(4-(4-((3-chloro-4-(pyridin-4- ylmethoxy)phenyl)amino)-7H-pyrrolo[2,3- d]pyrimidin-5-yl)piperidin-1-yl)prop-2-en-1- one |
| 128 | 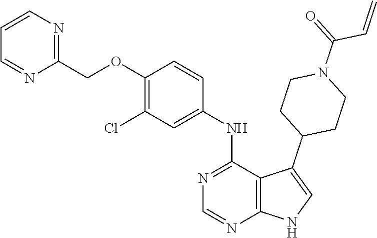 [SMILES: C=CC(=O)N1CCC(c2c[nH]c3ncnc(Nc4ccc(OCc5ncccn5)c(Cl)c4)c23)CC1] | 1-(4-(4-((3-chloro-4-(pyrimidin-2- ylmethoxy)phenyl)amino)-7H-pyrrolo[2,3- d]pyrimidin-5-yl)piperidin-1-yl)prop-2-en-1- one |
| 129 | 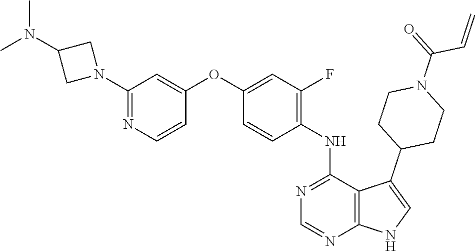 [SMILES: C=CC(=O)N1CCC(c2c[nH]c3ncnc(Nc4ccc(Oc5ccnc(N6CC(N(C)C)C6)c5)cc4F)c23)CC1] | 1-(4-(4-((4-((2-(3-(dimethylamino)azetidin-1- yl)pyridin-4-yl)oxy)-2-fluorophenyl)amino)- 7H-pyrrolo[2,3-d]pyrimidin-5-yl)piperidin-1- yl)prop-2-en-1-one |
| 130 | 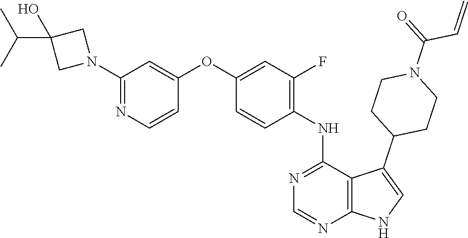 [SMILES: C=CC(=O)N1CCC(c2c[nH]c3ncnc(Nc4ccc(Oc5ccnc(N6CC(O)(C(C)C)C6)c5)cc4F)c23)CC1] | 1-(4-(4-((2-fluoro-4-((2-(3-hydroxy-3- isopropylazetidin-1-yl)pyridin-4- yl)oxy)phenyl)amino)-7H-pyrrolo[2,3- d]pyrimidin-5-yl)piperidin-1-yl)prop-2-en-1- one |
| 131 | 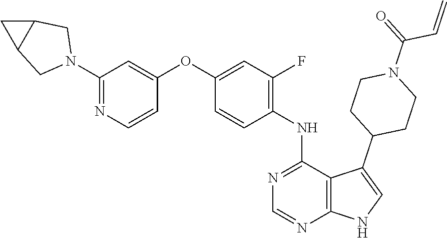 [SMILES: C=CC(=O)N1CCC(c2c[nH]c3ncnc(Nc4ccc(Oc5ccnc(N6CC7CC7C6)c5)cc4F)c23)CC1] | 1-(4-(4-((4-((2-(3-azabicyclo[3.1.0]hexan-3- yl)pyridin-4-yl)oxy)-2-fluorophenyl)amino)- 7H-pyrrolo[2,3-d]pyrimidin-5-yl)piperidin-1- yl)prop-2-en-1-one |
| 132 | 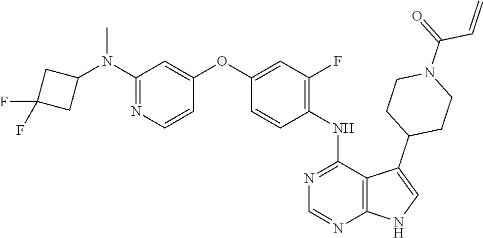 [SMILES: C=CC(=O)N1CCC(c2c[nH]c3ncnc(Nc4ccc(Oc5ccnc(N(C)C6CC(F)(F)C6)c5)cc4F)c23)CC1] | 1-(4-(4-((4-((2-((3,3- difluorocycloButyl)(methyl)amino)pyridin-4- yl)oxy)-2-fluorophenyl)amino)-7H- pyrrolo[2,3-d]pyrimidin-5-yl)piperidin-1- yl)prop-2-en-1-one |
| 133 | 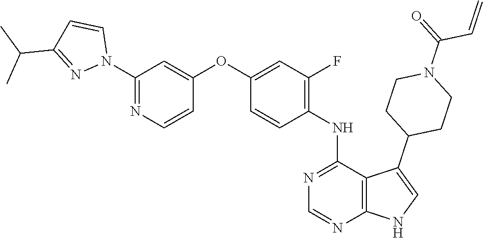 [SMILES: C=CC(=O)N1CCC(c2c[nH]c3ncnc(Nc4ccc(Oc5ccnc(-n6ccc(C(C)C)n6)c5)cc4F)c23)CC1] | 1-(4-(4-((2-fluoro-4-((2-(3-isopropyl-1H- pyrazol-1-yl)pyridin-4-yl)oxy)phenyl)amino)- 7H-pyrrolo[2,3-d]pyrimidin-5-yl)piperidin-1- yl)prop-2-en-1-one |
| 134 | 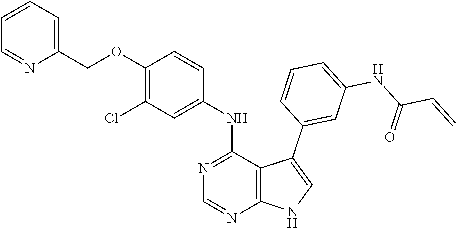 [SMILES: C=CC(=O)Nc1cccc(-c2c[nH]c3ncnc(Nc4ccc(OCc5ccccn5)c(Cl)c4)c23)c1] | 1-(4-(4-((2-fluoro-4-((2-(pyridin-2- ylamino)pyridin-4-yl)oxy)phenyl)amino)-7H- pyrrolo[2,3-d]pyrimidin-5-yl)piperidin-1- yl)prop-2-en-1-one |
| 135 | 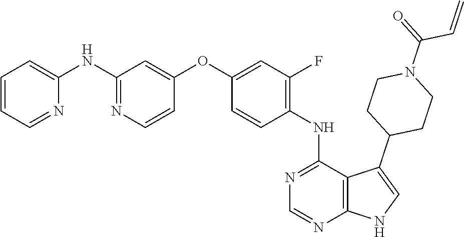 [SMILES: C=CC(=O)N1CCC(c2c[nH]c3ncnc(Nc4ccc(Oc5ccnc(Nc6ccccn6)c5)cc4F)c23)CC1] | 1-(4-(4-((2-fluoro-4-((2-(pyridin-2- ylamino)pyridin-4-yl)oxy)phenyl)amino)-7H- pyrrolo[2,3-d]pyrimidin-5-yl)piperidin-1- yl)prop-2-en-1-one |
| 136 | 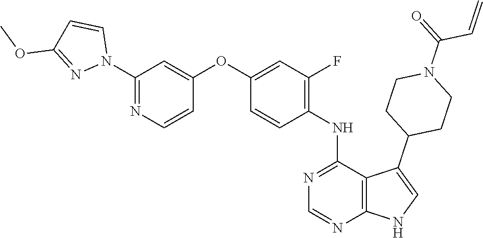 [SMILES: C=CC(=O)N1CCC(c2c[nH]c3ncnc(Nc4ccc(Oc5ccnc(-n6ccc(OC)n6)c5)cc4F)c23)CC1] | 1-(4-(4-((2-fluoro-4-((2-(3-methoxy-1H- pyrazol-1-yl)pyridin-4-yl)oxy)phenyl)amino)- 7H-pyrrolo[2,3-d]pyrimidin-5-yl)piperidin-1- yl)prop-2-en-1-one |
| 137 | 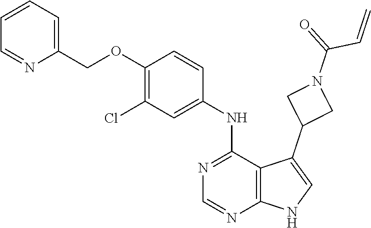 [SMILES: C=CC(=O)N1CC(c2c[nH]c3ncnc(Nc4ccc(OCc5ccccn5)c(Cl)c4)c23)C1] | 1-(3-(4-((3-chloro-4-(pyridin-2- ylmethoxy)phenyl)amino)-7H-pyrrolo[2,3- d]pyrimidin-5-yl)azetidin-1-yl)prop-2-en-1- one |
| 138 | 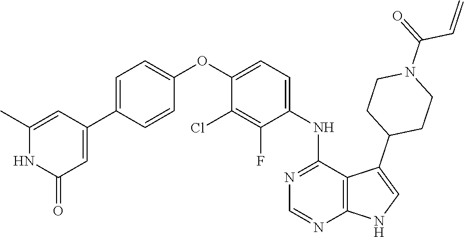 [SMILES: C=CC(=O)N1CCC(c2c[nH]c3ncnc(Nc4ccc(Oc5ccc(-c6cc(C)[nH]c(=O)c6)cc5)c(Cl)c4F)c23)CC1] | 4-(4-(4-((5-(1-acryloylpiperidin-4-yl)-7H- pyrrolo[2,3-d]pyrimidin-4-yl)amino)-2-chloro- 3-fluorophenoxy)phenyl)-6-methylpyridin- 2(1H)-one |
| 139 | 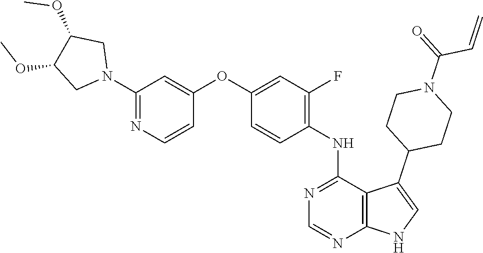 [SMILES: C=CC(=O)N1CCC(c2c[nH]c3ncnc(Nc4ccc(Oc5ccnc(N6C[C@H](OC)[C@H](OC)C6)c5)cc4F)c23)CC1] | 1-(4-(4-((4-((2-((3R,4S)-3,4- dimethoxypyrrolidin-1-yl)pyridin-4-yl)oxy)-2- fluorophenyl)amino)-7H-pyrrolo[2,3- d]pyrimidin-5-yl)piperidin-1-yl)prop-2-en-1- one |
| 140 | 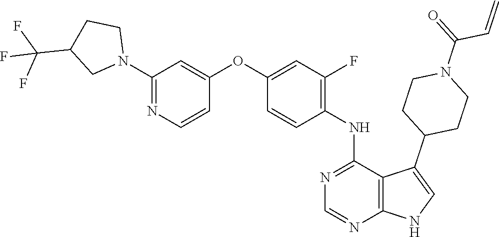 [SMILES: C=CC(=O)N1CCC(c2c[nH]c3ncnc(Nc4ccc(Oc5ccnc(N6CCC(C(F)(F)F)C6)c5)cc4F)c23)CC1] | 1-(4-(4-((2-fluoro-4-((2-(3- (trifluoromethyl)pyrrolidin-1-yl)pyridin-4- yl)oxy)phenyl)amino)-7H-pyrrolo[2,3- d]pyrimidin-5-yl)piperidin-1-yl)prop-2-en-1- one |
| 141 | 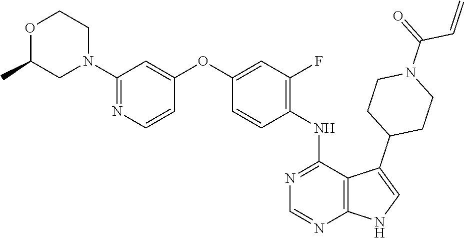 [SMILES: C=CC(=O)N1CCC(c2c[nH]c3ncnc(Nc4ccc(Oc5ccnc(N6CCO[C@H](C)C6)c5)cc4F)c23)CC1] | (S)-1-(4-(4-((2-fluoro-4-((2-(2- methylmorpholino)pyridin-4- yl)oxy)phenyl)amino)-7H-pyrrolo[2,3- d]pyrimidin-5-yl)piperidin-1-yl)prop-2-en-1- one |
| 142 | 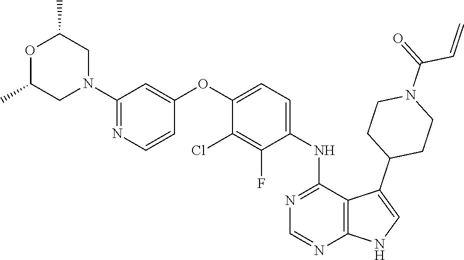 [SMILES: C=CC(=O)N1CCC(c2c[nH]c3ncnc(Nc4ccc(Oc5ccnc(N6C[C@@H](C)O[C@@H](C)C6)c5)c(Cl)c4F)c23)CC1] | 1-(4-(4-((3-chloro-4-((2-((2S,6R)-2,6- dimethylmorpholino)pyridin-4-yl)oxy)-2- fluorophenyl)amino)-7H-pyrrolo[2,3- d]pyrimidin-5-yl)piperidin-1-yl)prop-2-en-1- one |
| 143 | 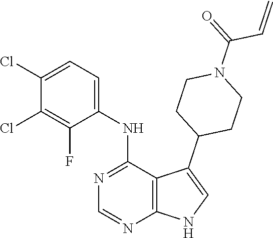 [SMILES: C=CC(=O)N1CCC(c2c[nH]c3ncnc(Nc4ccc(Cl)c(Cl)c4F)c23)CC1] | 1-(4-(4-((3,4-dichloro-2-fluorophenyl)amino)- 7H-pyrrolo[2,3-d]pyrimidin-5-yl)piperidin-1- yl)prop-2-en-1-one |
| 144 | 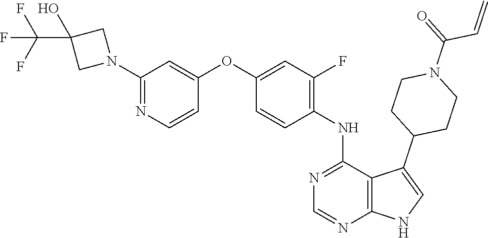 [SMILES: C=CC(=O)N1CCC(c2c[nH]c3ncnc(Nc4ccc(Oc5ccnc(N6CC(O)(C(F)(F)F)C6)c5)cc4F)c23)CC1] | 1-(4-(4-((2-fluoro-4-((2-(3-hydroxy-3- (trifluoromethyl)azetidin-1-yl)pyridin-4- yl)oxy)phenyl)amino)-7H-pyrrolo[2,3- d]pyrimidin-5-yl)piperidin-1-yl)prop-2-en-1- one |
| 145 | 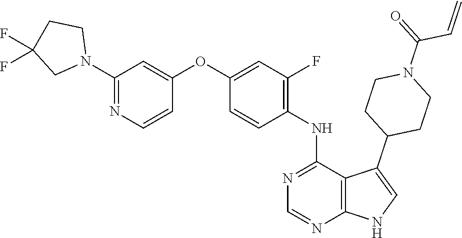 [SMILES: C=CC(=O)N1CCC(c2c[nH]c3ncnc(Nc4ccc(Oc5ccnc(N6CCC(F)(F)C6)c5)cc4F)c23)CC1] | 1-(4-(4-((4-((2-(3,3-difluoropyrrolidin-1- yl)pyridin-4-yl)oxy)-2-fluorophenyl)amino)- 7H-pyrrolo[2,3-d]pyrimidin-5-yl)piperidin-1- yl)prop-2-en-1-one |
| 146 | 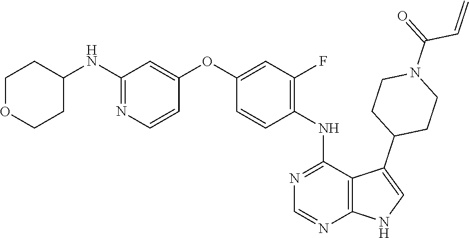 [SMILES: C=CC(=O)N1CCC(c2c[nH]c3ncnc(Nc4ccc(Oc5ccnc(NC6CCOCC6)c5)cc4F)c23)CC1] | 1-(4-(4-((2-fluoro-4-((2-((tetrahydro-2H- pyran-4-yl)amino)pyridin-4- yl)oxy)phenyl)amino)-7H-pyrrolo[2,3- d]pyrimidin-5-yl)piperidin-1-yl)prop-2-en-1- one |
| 147 | 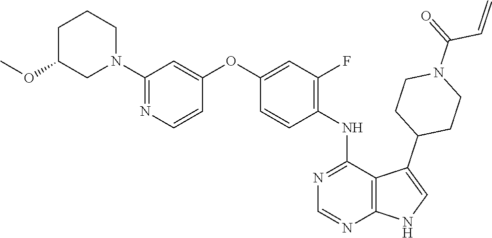 [SMILES: C=CC(=O)N1CCC(c2c[nH]c3ncnc(Nc4ccc(Oc5ccnc(N6CCC[C@@H](OC)C6)c5)cc4F)c23)CC1] | (R)-1-(4-(4-((2-fluoro-4-((2-(3- methoxypiperidin-1-yl)pyridin-4- yl)oxy)phenyl)amino)-7H-pyrrolo[2,3- d]pyrimidin-5-yl)piperidin-1-yl)prop-2-en-1- one |
| 148 | 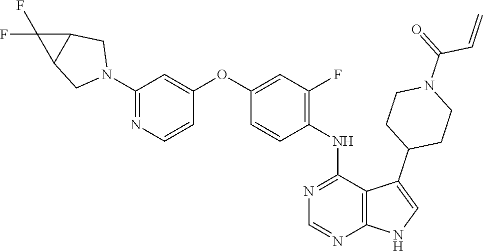 [SMILES: C=CC(=O)N1CCC(c2c[nH]c3ncnc(Nc4ccc(Oc5ccnc(N6CC7C(C6)C7(F)F)c5)cc4F)c23)CC1] | 1-(4-(4-((4-((2-(6,6-difluoro-3- azabicyclo[3.1.0]hexan-3-yl)pyridin-4- yl)oxy)-2-fluorophenyl)amino)-7H- pyrrolo[2,3-d]pyrimidin-5-yl)piperidin-1- yl)prop-2-en-1-one |
| 149 | 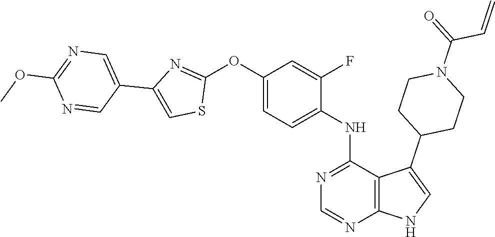 [SMILES: C=CC(=O)N1CCC(c2c[nH]c3ncnc(Nc4ccc(Oc5nc(-c6cnc(OC)nc6)cs5)cc4F)c23)CC1] | 1-(4-(4-((2-fluoro-4-((4-(2-methoxypyrimidin- 5-yl)thiazol-2-yl)oxy)phenyl)amino)-7H- pyrrolo[2,3-d]pyrimidin-5-yl)piperidin-1- yl)prop-2-en-1-one |
| 150 | 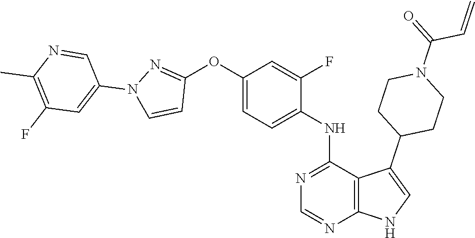 [SMILES: C=CC(=O)N1CCC(c2c[nH]c3ncnc(Nc4ccc(Oc5ccn(-c6cnc(C)c(F)c6)n5)cc4F)c23)CC1] | 1-(4-(4-((2-fluoro-4-((1-(5-fluoro-6- methylpyridin-3-yl)-1H-pyrazol-3- yl)oxy)phenyl)amino)-7H-pyrrolo[2,3- d]pyrimidin-5-yl)piperidin-1-yl)prop-2-en-1- one |
| 151 | 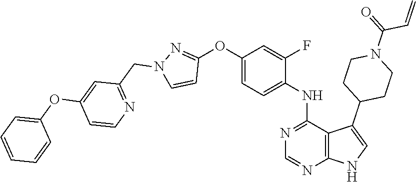 [SMILES: C=CC(=O)N1CCC(c2c[nH]c3ncnc(Nc4ccc(Oc5ccn(Cc6cc(Oc7ccccc7)ccn6)n5)cc4F)c23)CC1] | 1-(4-(4-((2-fluoro-4-((1-((4-phenoxypyridin-2- yl)methyl)-1H-pyrazol-3- yl)oxy)phenyl)amino)-7H-pyrrolo[2,3- d]pyrimidin-5-yl)piperidin-1-yl)prop-2-en-1- one |
| 152 | 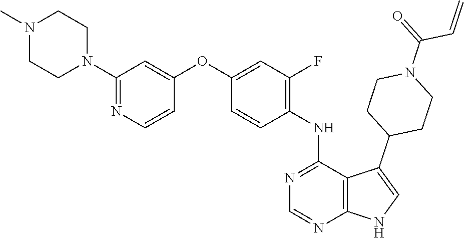 [SMILES: C=CC(=O)N1CCC(c2c[nH]c3ncnc(Nc4ccc(Oc5ccnc(N6CCN(C)CC6)c5)cc4F)c23)CC1] | 1-(4-(4-((2-fluoro-4-((2-(4-methylpiperazin-1- yl)pyridin-4-yl)oxy)phenyl)amino)-7H- pyrrolo[2,3-d]pyrimidin-5-yl)piperidin-1- yl)prop-2-en-1-one |
| 153 | 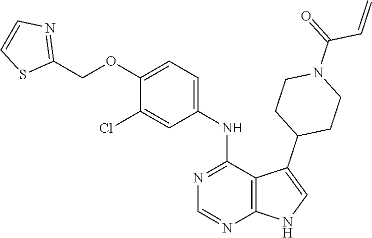 [SMILES: C=CC(=O)N1CCC(c2c[nH]c3ncnc(Nc4ccc(OCc5nccs5)c(Cl)c4)c23)CC1] | 1-(4-(4-((3-chloro-4-(thiazol-2- ylmethoxy)phenyl)amino)-7H-pyrrolo[2,3- d]pyrimidin-5-yl)piperidin-1-yl)prop-2-en-1- one |
| 154 | 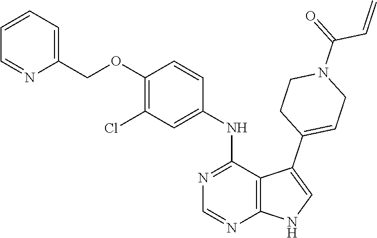 [SMILES: C=CC(=O)N1CC=C(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)-3,6-dihydropyridin-1(2H)- yl)prop-2-en-1-one |
| 155 | 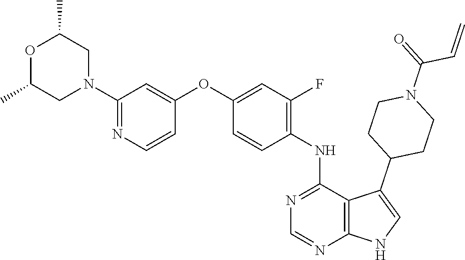 [SMILES: C=CC(=O)N1CCC(c2c[nH]c3ncnc(Nc4ccc(Oc5ccnc(N6C[C@@H](C)O[C@@H](C)C6)c5)cc4F)c23)CC1] | 1-(4-(4-((4-((2-((2S,6R)-2,6- dimethylmorpholino)pyridin-4-yl)oxy)-2- fluorophenyl)amino)-7H-pyrrolo[2,3- d]pyrimidin-5-yl)piperidin-1-yl)prop-2-en-1- one |
| 156 | 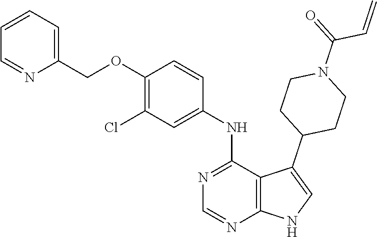 [SMILES: C=CC(=O)N1CCC(c2c[nH]c3ncnc(Nc4ccc(OCc5ccccn5)c(Cl)c4)c23)CC1] | 1-(4-(4-((3-chloro-4-(pyridin-2- ylmethoxy)phenyl)amino)-7H-pyrrolo[2,3- d]pyrimidin-5-yl)piperidin-1-yl)prop-2-en-1- one |
| 157 | 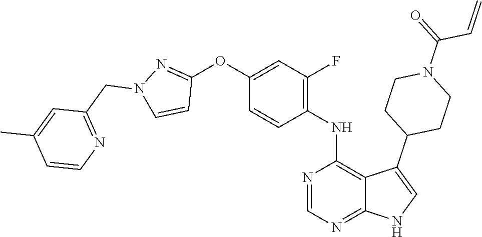 [SMILES: C=CC(=O)N1CCC(c2c[nH]c3ncnc(Nc4ccc(Oc5ccn(Cc6cc(C)ccn6)n5)cc4F)c23)CC1] | 1-(4-(4-((2-fluoro-4-((1-((4-methylpyridin-2- yl)methyl)-1H-pyrazol-3- yl)oxy)phenyl)amino)-7H-pyrrolo[2,3- d]pyrimidin-5-yl)piperidin-1-yl)prop-2-en-1- one |
| 158 | 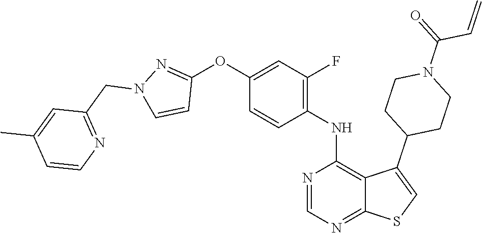 [SMILES: C=CC(=O)N1CCC(c2csc3ncnc(Nc4ccc(Oc5ccn(Cc6cc(C)ccn6)n5)cc4F)c23)CC1] | 1-(4-(4-((2-fluoro-4-((1-((4-methylpyridin-2- yl)methyl)-1H-pyrazol-3- yl)oxy)phenyl)amino)thieno[2,3-d]pyrimidin- 5-yl)piperidin-1-yl)prop-2-en-1-one |
| 159 | 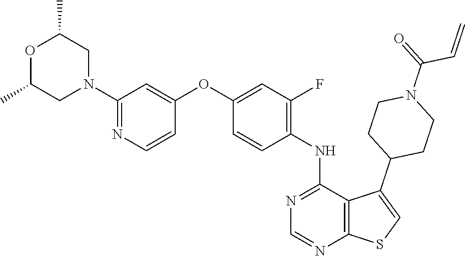 [SMILES: C=CC(=O)N1CCC(c2csc3ncnc(Nc4ccc(Oc5ccnc(N6C[C@@H](C)O[C@@H](C)C6)c5)cc4F)c23)CC1] | 1-(4-(4-((4-((2-((2R,6S)-2,6- dimethylmorpholino)pyridin-4-yl)oxy)-2- fluorophenyl)amino)thieno[2,3-d]pyrimidin-5- yl)piperidin-1-yl)prop-2-en-1-one |
| 160 | 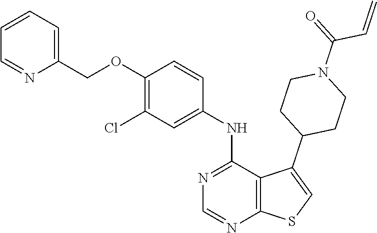 [SMILES: C=CC(=O)N1CCC(c2csc3ncnc(Nc4ccc(OCc5ccccn5)c(Cl)c4)c23)CC1] | 1-(4-(4-((3-chloro-4-(pyridin-2- ylmethoxy)phenyl)amino)thieno[2,3- d]pyrimidin-5-yl)piperidin-1-yl)prop-2-en-1- one |
| 161 | 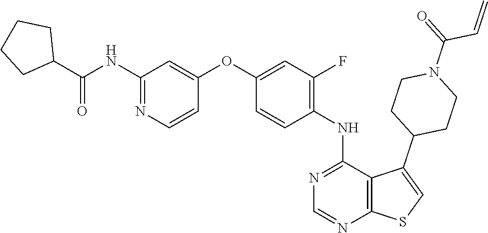 [SMILES: C=CC(=O)N1CCC(c2csc3ncnc(Nc4ccc(Oc5ccnc(NC(=O)C6CCCC6)c5)cc4F)c23)CC1] | N-(4-(4-((5-(1-acryloylpiperidin-4- yl)thieno[2,3-d]pyrimidin-4-yl)amino)-3- fluorophenoxy)pyridin-2- yl)cyclopentanecarboxamide |
| 162 | 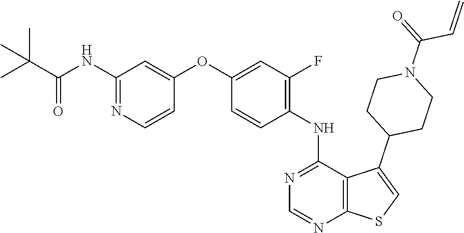 [SMILES: C=CC(=O)N1CCC(c2csc3ncnc(Nc4ccc(Oc5ccnc(NC(=O)C(C)(C)C)c5)cc4F)c23)CC1] | N-(4-(4-((5-(1-acryloylpiperidin-4- yl)thieno[2,3-d]pyrimidin-4-yl)amino)-3- fluorophenoxy)pyridin-2-yl)pivalamide |
| 163 | 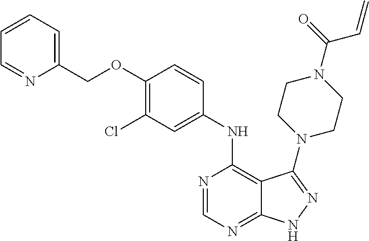 [SMILES: C=CC(=O)N1CCN(c2n[nH]c3ncnc(Nc4ccc(OCc5ccccn5)c(Cl)c4)c23)CC1] | 1-(4-(4-((3-chloro-4-(pyridin-2- ylmethoxy)phenyl)amino)-1H-pyrazolo[3,4- d]pyrimidin-3-yl)piperazin-1-yl)prop-2-en-1- one |
| 164 | 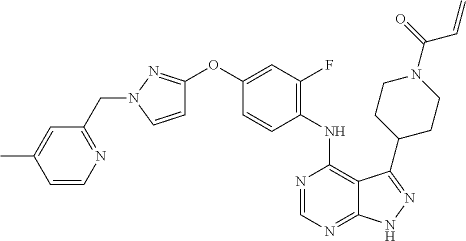 [SMILES: C=CC(=O)N1CCC(c2n[nH]c3ncnc(Nc4ccc(Oc5ccn(Cc6cc(C)ccn6)n5)cc4F)c23)CC1] | 1-(4-(4-((2-fluoro-4-((1-((4-methylpyridin-2- yl)methyl)-1H-pyrazol-3- yl)oxy)phenyl)amino)-1H-pyrazolo[3,4- d]pyrimidin-3-yl)piperidin-1-yl)prop-2-en-1- one |
| 165 | 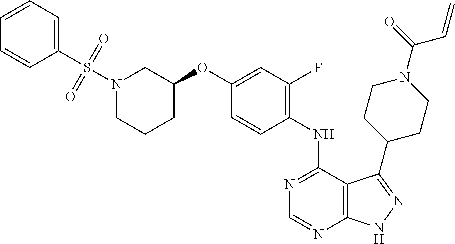 [SMILES: C=CC(=O)N1CCC(c2n[nH]c3ncnc(Nc4ccc(O[C@H]5CCCN(S(=O)(=O)c6ccccc6)C5)cc4F)c23)CC1] | (S)-1-(4-(4-((2-fluoro-4-((1- (phenylsulfonyl)piperidin-3- yl)oxy)phenyl)amino)-1H-pyrazolo[3,4- d]pyrimidin-3-yl)piperidin-1-yl)prop-2-en-1- one |
| 166 | 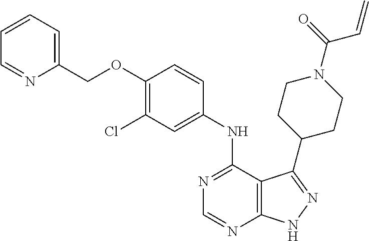 [SMILES: C=CC(=O)N1CCC(c2n[nH]c3ncnc(Nc4ccc(OCc5ccccn5)c(Cl)c4)c23)CC1] | 1-(4-(4-((3-chloro-4-(pyridin-2- ylmethoxy)phenyl)amino)-1H-pyrazolo[3,4- d]pyrimidin-3-yl)piperidin-1-yl)prop-2-en-1- one |
| 167 | 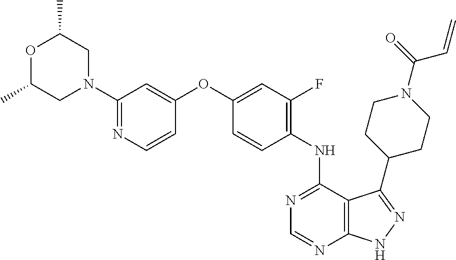 [SMILES: C=CC(=O)N1CCC(c2n[nH]c3ncnc(Nc4ccc(Oc5ccnc(N6C[C@@H](C)O[C@@H](C)C6)c5)cc4F)c23)CC1] | 1-(4-(4-((4-((2-((2R,6S)-2,6- dimethylmorpholino)pyridin-4-yl)oxy)-2- fluorophenyl)amino)-1H-pyrazolo[3,4- d]pyrimidin-3-yl)piperidin-1-yl)prop-2-en-1- one |
| 168 | 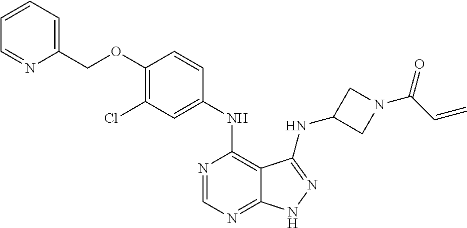 [SMILES: C=CC(=O)N1CC(Nc2n[nH]c3ncnc(Nc4ccc(OCc5ccccn5)c(Cl)c4)c23)C1] | 1-(3-((4-((3-chloro-4-(pyridin-2- ylmethoxy)phenyl)amino)-1H-pyrazolo[3,4- d]pyrimidin-3-yl)amino)azetidin-1-yl)prop-2- en-1-one |
| 169 | 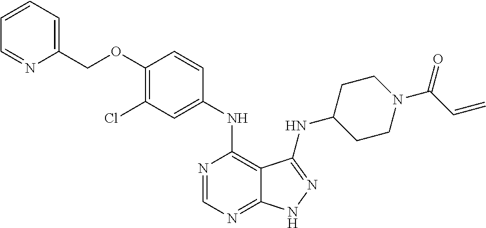 [SMILES: C=CC(=O)N1CCC(Nc2n[nH]c3ncnc(Nc4ccc(OCc5ccccn5)c(Cl)c4)c23)CC1] | 1-(4-((4-((3-chloro-4-(pyridin-2- ylmethoxy)phenyl)amino)-1H-pyrazolo[3,4- d]pyrimidin-3-yl)amino)piperidin-1-yl)prop-2- en-1-one |
| 170 | 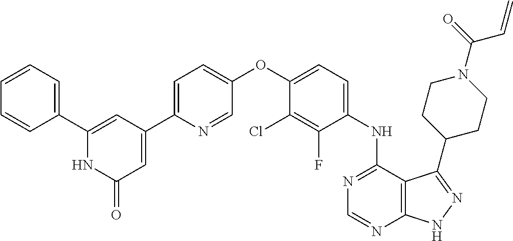 [SMILES: C=CC(=O)N1CCC(c2n[nH]c3ncnc(Nc4ccc(Oc5ccc(-c6cc(-c7ccccc7)[nH]c(=O)c6)nc5)c(Cl)c4F)c23)CC1] | 5-(4-((3-(1-acryloylpiperidin-4-yl)-1H- pyrazolo[3,4-d]pyrimidin-4-yl)amino)-2- chloro-3-fluorophenoxy)-6′-phenyl-[2,4′- bipyridin]-2′(1′H)-one |
| 171 | 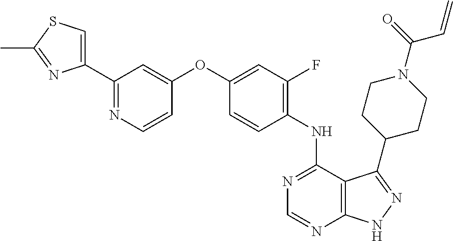 [SMILES: C=CC(=O)N1CCC(c2n[nH]c3ncnc(Nc4ccc(Oc5ccnc(-c6csc(C)n6)c5)cc4F)c23)CC1] | 1-(4-(4-((2-fluoro-4-((2-(2-methylthiazol-4- yl)pyridin-4-yl)oxy)phenyl)amino)-1H- pyrazolo[3,4-d]pyrimidin-3-yl)piperidin-1- yl)prop-2-en-1-one |
| 172 | 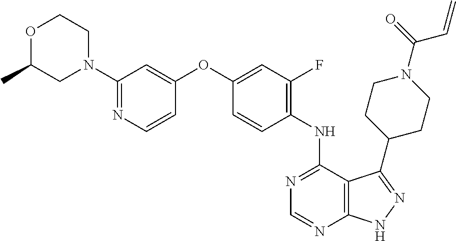 [SMILES: C=CC(=O)N1CCC(c2n[nH]c3ncnc(Nc4ccc(Oc5ccnc(N6CCO[C@H](C)C6)c5)cc4F)c23)CC1] | (R)-1-(4-(4-((2-fluoro-4-((2-(2- methylmorpholino)pyridin-4- yl)oxy)phenyl)amino)-1H-pyrazolo[3,4- d]pyrimidin-3-yl)piperidin-1-yl)prop-2-en-1- one |
| 173 | 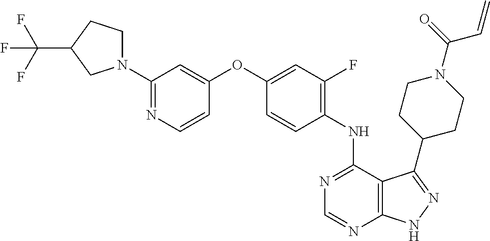 [SMILES: C=CC(=O)N1CCC(c2n[nH]c3ncnc(Nc4ccc(Oc5ccnc(N6CCC(C(F)(F)F)C6)c5)cc4F)c23)CC1] | 1-(4-(4-((2-fluoro-4-((2-(3- (trifluoromethyl)pyrrolidin-1-yl)pyridin-4- yl)oxy)phenyl)amino)-1H-pyrazolo[3,4- d]pyrimidin-3-yl)piperidin-1-yl)prop-2-en-1- one |
| 174 | 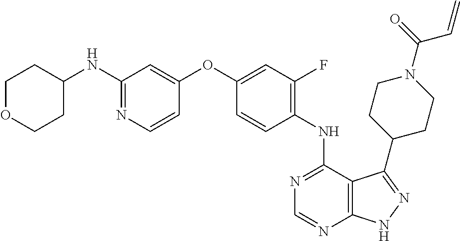 [SMILES: C=CC(=O)N1CCC(c2n[nH]c3ncnc(Nc4ccc(Oc5ccnc(NC6CCOCC6)c5)cc4F)c23)CC1] | 1-(4-(4-((2-fluoro-4-((2-((tetrahydro-2H- pyran-4-yl)amino)pyridin-4- yl)oxy)phenyl)amino)-1H-pyrazolo[3,4- d]pyrimidin-3-yl)piperidin-1-yl)prop-2-en-1- one |
| 175 | 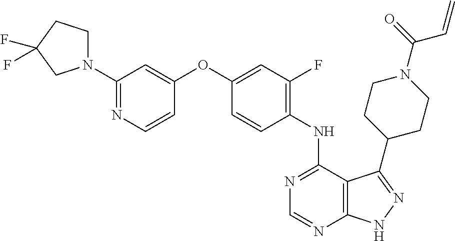 [SMILES: C=CC(=O)N1CCC(c2n[nH]c3ncnc(Nc4ccc(Oc5ccnc(N6CCC(F)(F)C6)c5)cc4F)c23)CC1] | 1-(4-(4-((4-((2-(3,3-difluoropyrrolidin-1- yl)pyridin-4-yl)oxy)-2-fluorophenyl)amino)- 1H-pyrazolo[3,4-d]pyrimidin-3-yl)piperidin-1- yl)prop-2-en-1-one |
| 176 | 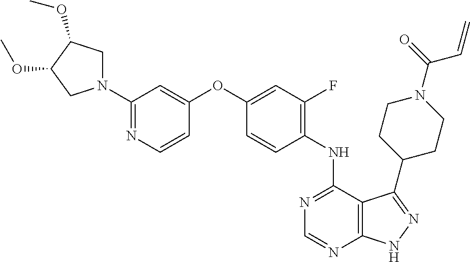 [SMILES: C=CC(=O)N1CCC(c2n[nH]c3ncnc(Nc4ccc(Oc5ccnc(N6C[C@H](OC)[C@H](OC)C6)c5)cc4F)c23)CC1] | 1-(4-(4-((4-((2-((3S,4R)-3,4- dimethoxypyrrolidin-1-yl)pyridin-4-yl)oxy)-2- fluorophenyl)amino)-1H-pyrazolo[3,4- d]pyrimidin-3-yl)piperidin-1-yl)prop-2-en-1- one |
| 177 | 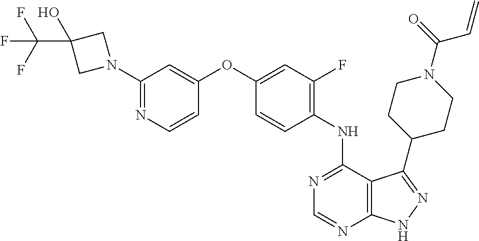 [SMILES: C=CC(=O)N1CCC(c2n[nH]c3ncnc(Nc4ccc(Oc5ccnc(N6CC(O)(C(F)(F)F)C6)c5)cc4F)c23)CC1] | 1-(4-(4-((2-fluoro-4-((2-(3-hydroxy-3- (trifluoromethyl)azetidin-1-yl)pyridin-4- yl)oxy)phenyl)amino)-1H-pyrazolo[3,4- d]pyrimidin-3-yl)piperidin-1-yl)prop-2-en-1- one |
| 178 | 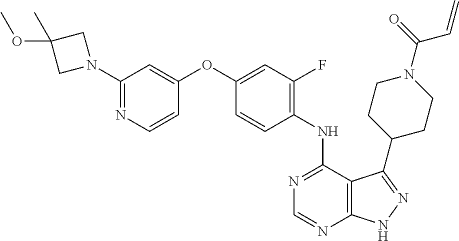 [SMILES: C=CC(=O)N1CCC(c2n[nH]c3ncnc(Nc4ccc(Oc5ccnc(N6CC(C)(OC)C6)c5)cc4F)c23)CC1] | 1-(4-(4-((2-fluoro-4-((2-(3-methoxy-3- methylazetidin-1-yl)pyridin-4- yl)oxy)phenyl)amino)-1H-pyrazolo[3,4- d]pyrimidin-3-yl)piperidin-1-yl)prop-2-en-1- one |
| 179 | 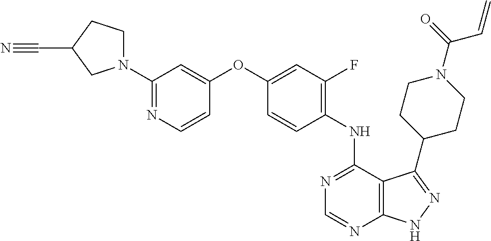 [SMILES: C=CC(=O)N1CCC(c2n[nH]c3ncnc(Nc4ccc(Oc5ccnc(N6CCC(C#N)C6)c5)cc4F)c23)CC1] | 1-(4-(4-((3-(1-acryloylpiperidin-4-yl)-1H- pyrazolo[3,4-d]pyrimidin-4-yl)amino)-3- fluorophenoxy)pyridin-2-yl)pyrrolidine-3- carbonitrile |
| 180 | 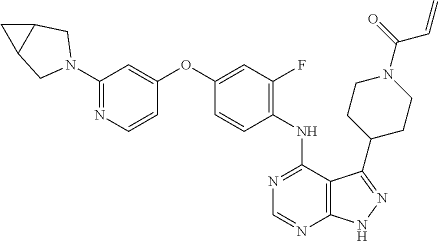 [SMILES: C=CC(=O)N1CCC(c2n[nH]c3ncnc(Nc4ccc(Oc5ccnc(N6CC7CC7C6)c5)cc4F)c23)CC1] | 1-(4-(4-((4-((2-(3-azabicyclo[3.1.0]hexan-3- yl)pyridin-4-yl)oxy)-2-fluorophenyl)amino)- 1H-pyrazolo[3,4-d]pyrimidin-3-yl)piperidin-1- yl)prop-2-en-1-one |
| 181 | 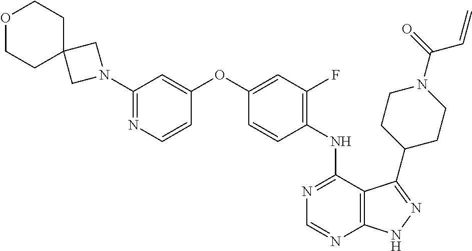 [SMILES: C=CC(=O)N1CCC(c2n[nH]c3ncnc(Nc4ccc(Oc5ccnc(N6CC7(CCOCC7)C6)c5)cc4F)c23)CC1] | 1-(4-(4-((4-((2-(7-oxa-2-azaspiro[3.5]nonan- 2-yl)pyridin-4-yl)oxy)-2-fluorophenyl)amino)- 1H-pyrazolo[3,4-d]pyrimidin-3-yl)piperidin-1- yl)prop-2-en-1-one |
| 182 | 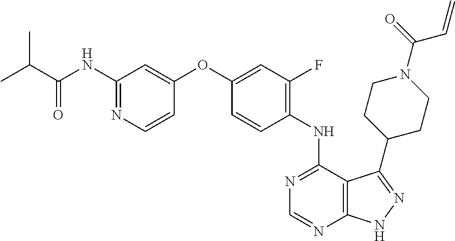 [SMILES: C=CC(=O)N1CCC(c2n[nH]c3ncnc(Nc4ccc(Oc5ccnc(NC(=O)C(C)C)c5)cc4F)c23)CC1] | N-(4-(4-((3-(1-acryloylpiperidin-4-yl)-1H- pyrazolo[3,4-d]pyrimidin-4-yl)amino)-3- fluorophenoxy)pyridin-2-yl)isobutyramide |
| 183 | 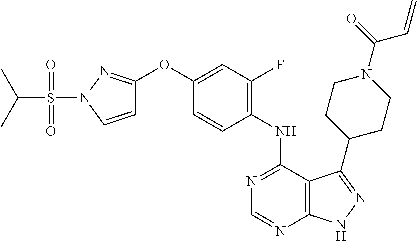 [SMILES: C=CC(=O)N1CCC(c2n[nH]c3ncnc(Nc4ccc(Oc5ccn(S(=O)(=O)C(C)C)n5)cc4F)c23)CC1] | 1-(4-(4-((2-fluoro-4-((1-(isopropylsulfonyl)- 1H-pyrazol-3-yl)oxy)phenyl)amino)-1H- pyrazolo[3,4-d]pyrimidin-3-yl)piperidin-1- yl)prop-2-en-1-one |
| 184 | 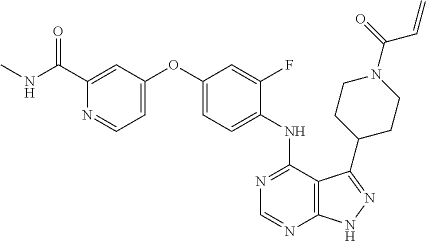 [SMILES: C=CC(=O)N1CCC(c2n[nH]c3ncnc(Nc4ccc(Oc5ccnc(C(=O)NC)c5)cc4F)c23)CC1] | 4-(4-((3-(1-acryloylpiperidin-4-yl)-1H- pyrazolo[3,4-d]pyrimidin-4-yl)amino)-3- fluorophenoxy)-N-methylpicolinamide |
| 185 | 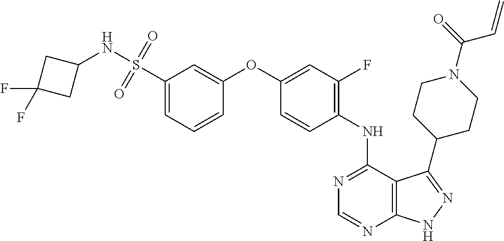 [SMILES: C=CC(=O)N1CCC(c2n[nH]c3ncnc(Nc4ccc(Oc5cccc(S(=O)(=O)NC6CC(F)(F)C6)c5)cc4F)c23)CC1] | 3-(4-((3-(1-acryloylpiperidin-4-yl)-1H- pyrazolo[3,4-d]pyrimidin-4-yl)amino)-3- fluorophenoxy)-N-(3,3- difluorocycloButyl)benzenesulfonamide |
| 186 | 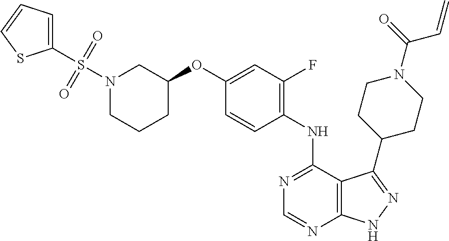 [SMILES: C=CC(=O)N1CCC(c2n[nH]c3ncnc(Nc4ccc(O[C@H]5CCCN(S(=O)(=O)c6cccs6)C5)cc4F)c23)CC1] | (S)-1-(4-(4-((2-fluoro-4-((1-(thiophen-2- ylsulfonyl)piperidin-3-yl)oxy)phenyl)amino)- 1H-pyrazolo[3,4-d]pyrimidin-3-yl)piperidin-1- yl)prop-2-en-1-one |
| 187 | 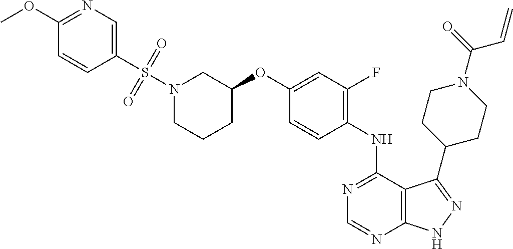 [SMILES: C=CC(=O)N1CCC(c2n[nH]c3ncnc(Nc4ccc(O[C@H]5CCCN(S(=O)(=O)c6ccc(OC)nc6)C5)cc4F)c23)CC1] | (S)-1-(4-(4-((2-fluoro-4-((1-((6- methoxypyridin-3-yl)sulfonyl)piperidin-3- yl)oxy)phenyl)amino)-1H-pyrazolo[3,4- d]pyrimidin-3-yl)piperidin-1-yl)prop-2-en-1- one |
| 188 | 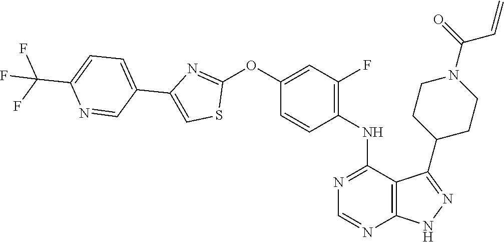 [SMILES: C=CC(=O)N1CCC(c2n[nH]c3ncnc(Nc4ccc(Oc5nc(-c6ccc(C(F)(F)F)nc6)cs5)cc4F)c23)CC1] | 1-(4-(4-((2-fluoro-4-((4-(6- (trifluoromethyl)pyridin-3-yl)thiazol-2- yl)oxy)phenyl)amino)-1H-pyrazolo[3,4- d]pyrimidin-3-yl)piperidin-1-yl)prop-2-en-1- one |
| 189 | 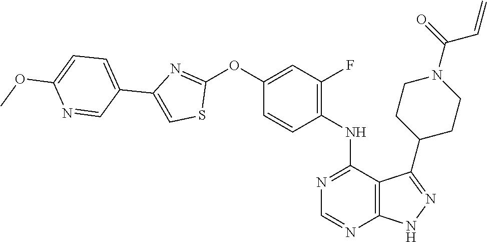 [SMILES: C=CC(=O)N1CCC(c2n[nH]c3ncnc(Nc4ccc(Oc5nc(-c6ccc(OC)nc6)cs5)cc4F)c23)CC1] | 1-(4-(4-((2-fluoro-4-((4-(6-methoxypyridin-3- yl)thiazol-2-yl)oxy)phenyl)amino)-1H- pyrazolo[3,4-d]pyrimidin-3-yl)piperidin-1- yl)prop-2-en-1-one |
| 190 | 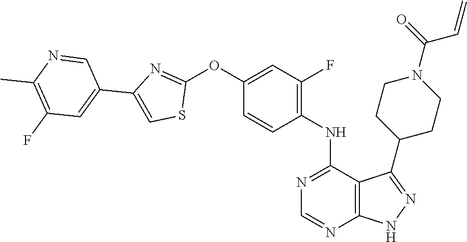 [SMILES: C=CC(=O)N1CCC(c2n[nH]c3ncnc(Nc4ccc(Oc5nc(-c6cnc(C)c(F)c6)cs5)cc4F)c23)CC1] | 1-(4-(4-((2-fluoro-4-((4-(5-fluoro-6- methylpyridin-3-yl)thiazol-2- yl)oxy)phenyl)amino)-1H-pyrazolo[3,4- d]pyrimidin-3-yl)piperidin-1-yl)prop-2-en-1- one |
| 191 | 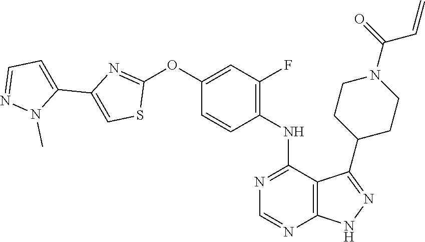 [SMILES: C=CC(=O)N1CCC(c2n[nH]c3ncnc(Nc4ccc(Oc5nc(-c6ccnn6C)cs5)cc4F)c23)CC1] | 1-(4-(4-((2-fluoro-4-((4-(1-methyl-1H- pyrazol-5-yl)thiazol-2-yl)oxy)phenyl)amino)- 1H-pyrazolo[3,4-d]pyrimidin-3-yl)piperidin-1- yl)prop-2-en-1-one |
| 192 | 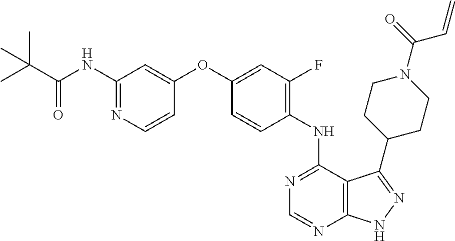 [SMILES: C=CC(=O)N1CCC(c2n[nH]c3ncnc(Nc4ccc(Oc5ccnc(NC(=O)C(C)(C)C)c5)cc4F)c23)CC1] | N-(4-(4-((3-(1-acryloylpiperidin-4-yl)-1H- pyrazolo[3,4-d]pyrimidin-4-yl)amino)-3- fluorophenoxy)pyridin-2-yl)pivalamide |
| 193 | 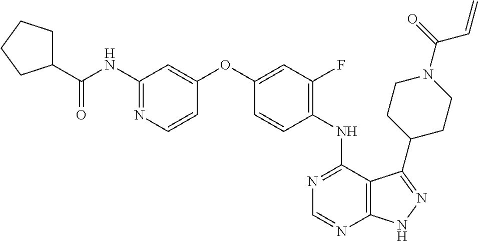 [SMILES: C=CC(=O)N1CCC(c2n[nH]c3ncnc(Nc4ccc(Oc5ccnc(NC(=O)C6CCCC6)c5)cc4F)c23)CC1] | N-(4-(4-((3-(1-acryloylpiperidin-4-yl)-1H- pyrazolo[3,4-d]pyrimidin-4-yl)amino)-3- fluorophenoxy)pyridin-2- yl)cyclopentanecarboxamide |
| 194 | 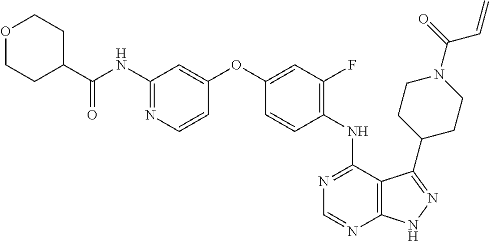 [SMILES: C=CC(=O)N1CCC(c2n[nH]c3ncnc(Nc4ccc(Oc5ccnc(NC(=O)C6CCOCC6)c5)cc4F)c23)CC1] | N-(4-(4-((3-(1-acryloylpiperidin-4-yl)-1H- pyrazolo[3,4-d]pyrimidin-4-yl)amino)-3- fluorophenoxy)pyridin-2-yl)tetrahydro-2H- pyran-4-carboxamide |
| 195 | 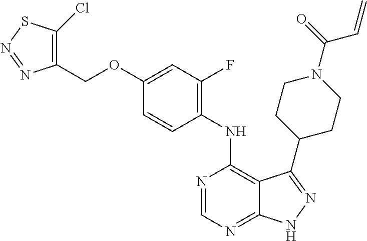 [SMILES: C=CC(=O)N1CCC(c2n[nH]c3ncnc(Nc4ccc(OCc5nnsc5Cl)cc4F)c23)CC1] | 1-(4-(4-((4-((5-chloro-1,2,3-thiadiazol-4- yl)methoxy)-2-fluorophenyl)amino)-1H- pyrazolo[3,4-d]pyrimidin-3-yl)piperidin-1- yl)prop-2-en-1-one |
| 196 | 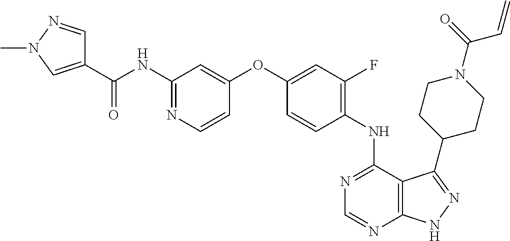 [SMILES: C=CC(=O)N1CCC(c2n[nH]c3ncnc(Nc4ccc(Oc5ccnc(NC(=O)c6cnn(C)c6)c5)cc4F)c23)CC1] | N-(4-(4-((3-(1-acryloylpiperidin-4-yl)-1H- pyrazolo[3,4-d]pyrimidin-4-yl)amino)-3- fluorophenoxy)pyridin-2-yl)-1-methyl-1H- pyrazole-4-carboxamide |
| 197 | 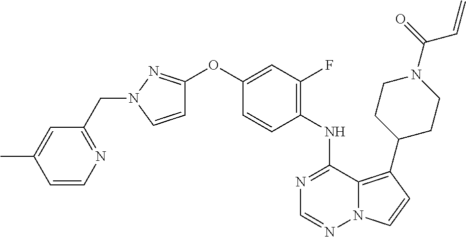 [SMILES: C=CC(=O)N1CCC(c2ccn3ncnc(Nc4ccc(Oc5ccn(Cc6cc(C)ccn6)n5)cc4F)c23)CC1] | 1-(4-(4-((2-fluoro-4-((1-((4-methylpyridin-2- yl)methyl)-1H-pyrazol-3- yl)oxy)phenyl)amino)pyrrolo[2,1- f][1,2,4]triazin-5-yl)piperidin-1-yl)prop-2-en- 1-one |
| 198 | 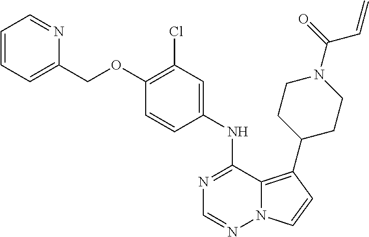 [SMILES: C=CC(=O)N1CCC(c2ccn3ncnc(Nc4ccc(OCc5ccccn5)c(Cl)c4)c23)CC1] | 1-(4-(4-((3-chloro-4-(pyridin-2- ylmethoxy)phenyl)amino)pyrrolo[2,1- f][1,2,4]triazin-5-yl)piperidin-1-yl)prop-2-en- 1-one |
| 199 | 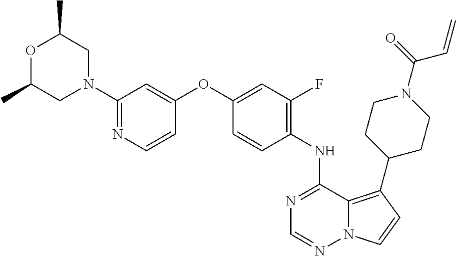 [SMILES: C=CC(=O)N1CCC(c2ccn3ncnc(Nc4ccc(Oc5ccnc(N6C[C@@H](C)O[C@@H](C)C6)c5)cc4F)c23)CC1] | 1-(4-(4-((4-((2-((2R,6S)-2,6- dimethylmorpholino)pyridin-4-yl)oxy)-2- fluorophenyl)amino)pyrrolo[2,1- f][1,2,4]triazin-5-yl)piperidin-1-yl)prop-2-en- 1-one |
| 200 | 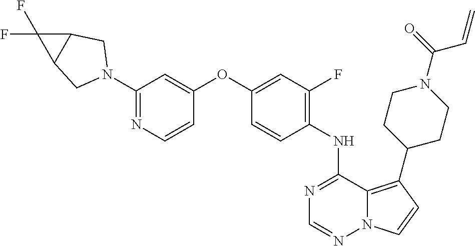 [SMILES: C=CC(=O)N1CCC(c2ccn3ncnc(Nc4ccc(Oc5ccnc(N6CC7C(C6)C7(F)F)c5)cc4F)c23)CC1] | 1-(4-(4-((4-((2-(6,6-difluoro-3- azabicyclo[3.1.0]hexan-3-yl)pyridin-4- yl)oxy)-2-fluorophenyl)amino)pyrrolo[2,1- f][1,2,4]triazin-5-yl)piperidin-1-yl)prop-2-en- 1-one |
| 201 | 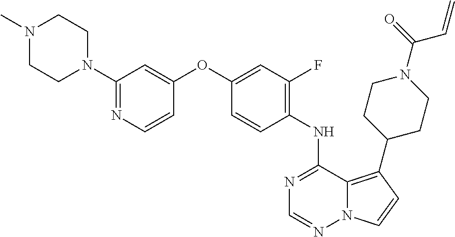 [SMILES: C=CC(=O)N1CCC(c2ccn3ncnc(Nc4ccc(Oc5ccnc(N6CCN(C)CC6)c5)cc4F)c23)CC1] | 1-(4-(4-((2-fluoro-4-((2-(4-methylpiperazin-1- yl)pyridin-4-yl)oxy)phenyl)amino)pyrrolo[2,1- f][1,2,4]triazin-5-yl)piperidin-1-yl)prop-2-en- 1-one |
| 202 | 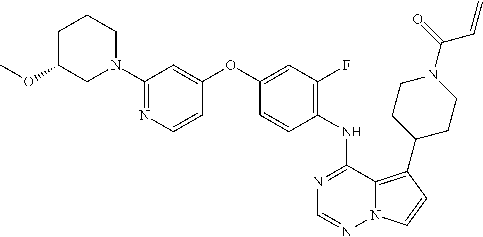 [SMILES: C=CC(=O)N1CCC(c2ccn3ncnc(Nc4ccc(Oc5ccnc(N6CCC[C@@H](OC)C6)c5)cc4F)c23)CC1] | (R)-1-(4-(4-((2-fluoro-4-((2-(3- methoxypiperidin-1-yl)pyridin-4- yl)oxy)phenyl)amino)pyrrolo[2,1- f][1,2,4]triazin-5-yl)piperidin-1-yl)prop-2-en- 1-one |
| 203 | 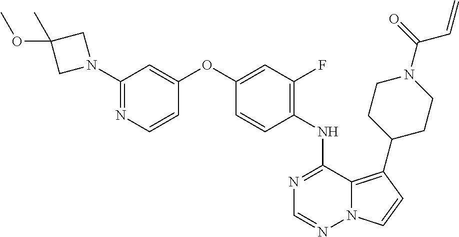 [SMILES: C=CC(=O)N1CCC(c2ccn3ncnc(Nc4ccc(Oc5ccnc(N6CC(C)(OC)C6)c5)cc4F)c23)CC1] | 1-(4-(4-((2-fluoro-4-((2-(3-methoxy-3- methylazetidin-1-yl)pyridin-4- yl)oxy)phenyl)amino)pyrrolo[2,1- f][1,2,4]triazin-5-yl)piperidin-1-yl)prop-2-en- 1-one |
| 204 | 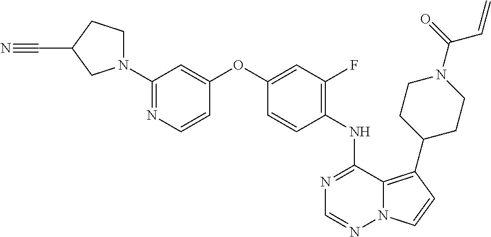 [SMILES: C=CC(=O)N1CCC(c2ccn3ncnc(Nc4ccc(Oc5ccnc(N6CCC(C#N)C6)c5)cc4F)c23)CC1] | 1-(4-(4-((5-(1-acryloylpiperidin-4- yl)pyrrolo[2,1-f][1,2,4]triazin-4-yl)amino)-3- fluorophenoxy)pyridin-2-yl)pyrrolidine-3- carbonitrile |
| 205 | 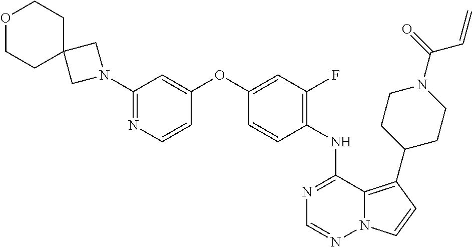 [SMILES: C=CC(=O)N1CCC(c2ccn3ncnc(Nc4ccc(Oc5ccnc(N6CC7(CCOCC7)C6)c5)cc4F)c23)CC1] | 1-(4-(4-((4-((2-(7-oxa-2-azaspiro[3.5]nonan- 2-yl)pyridin-4-yl)oxy)-2- fluorophenyl)amino)pyrrolo[2,1- f][1,2,4]triazin-5-yl)piperidin-1-yl)prop-2-en- 1-one |
| 206 | 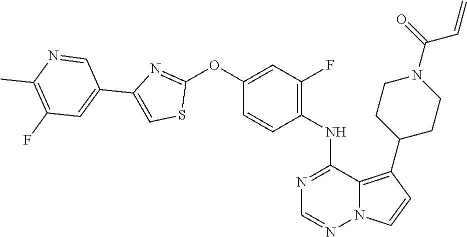 [SMILES: C=CC(=O)N1CCC(c2ccn3ncnc(Nc4ccc(Oc5nc(-c6cnc(C)c(F)c6)cs5)cc4F)c23)CC1] | 1-(4-(4-((2-fluoro-4-((4-(5-fluoro-6- methylpyridin-3-yl)thiazol-2- yl)oxy)phenyl)amino)pyrrolo[2,1- f][1,2,4]triazin-5-yl)piperidin-1-yl)prop-2-en- 1-one |
| 207 | 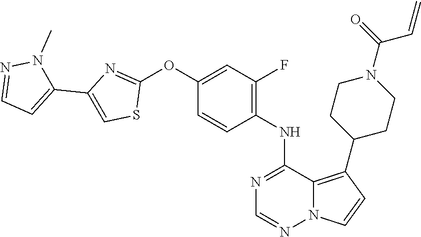 [SMILES: C=CC(=O)N1CCC(c2ccn3ncnc(Nc4ccc(Oc5nc(-c6ccnn6C)cs5)cc4F)c23)CC1] | 1-(4-(4-((2-fluoro-4-((4-(1-methyl-1H- pyrazol-5-yl)thiazol-2- yl)oxy)phenyl)amino)pyrrolo[2,1- f][1,2,4]triazin-5-yl)piperidin-1-yl)prop-2-en- 1-one |
| 208 | 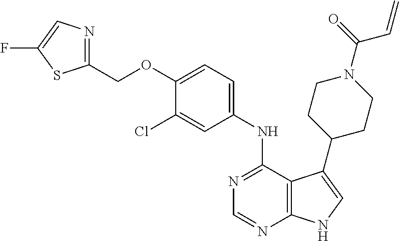 [SMILES: C=CC(=O)N1CCC(c2c[nH]c3ncnc(Nc4ccc(OCc5ncc(F)s5)c(Cl)c4)c23)CC1] | 1-(4-(4-((3-chloro-4-((5-fluorothiazol-2- yl)methoxy)phenyl)amino)-7H-pyrrolo[2,3- d]pyrimidin-5-yl)piperidin-1-yl)prop-2-en-1- one |
| 209 | 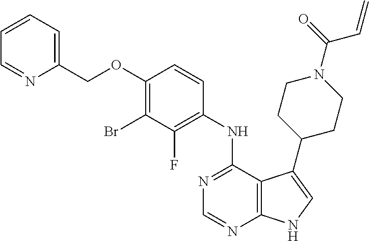 [SMILES: C=CC(=O)N1CCC(c2c[nH]c3ncnc(Nc4ccc(OCc5ccccn5)c(Br)c4F)c23)CC1] | 1-(4-(4-((3-bromo-2-fluoro-4-(pyridin-2- ylmethoxy)phenyl)amino)-7H-pyrrolo[2,3- d]pyrimidin-5-yl)piperidin-1-yl)prop-2-en-1- one |
| 210 | 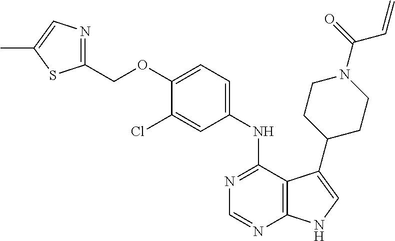 [SMILES: C=CC(=O)N1CCC(c2c[nH]c3ncnc(Nc4ccc(OCc5ncc(C)s5)c(Cl)c4)c23)CC1] | 1-(4-(4-((3-chloro-4-((5-methylthiazol-2- yl)methoxy)phenyl)amino)-7H-pyrrolo[2,3- d]pyrimidin-5-yl)piperidin-1-yl)prop-2-en-1- one |
| 211 | 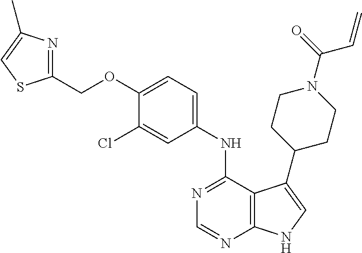 [SMILES: C=CC(=O)N1CCC(c2c[nH]c3ncnc(Nc4ccc(OCc5nc(C)cs5)c(Cl)c4)c23)CC1] | 1-(4-(4-((3-chloro-4-((4-methylthiazol-2- yl)methoxy)phenyl)amino)-7H-pyrrolo[2,3- d]pyrimidin-5-yl)piperidin-1-yl)prop-2-en-1- one |
| 212 | 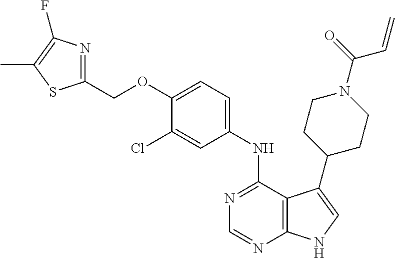 [SMILES: C=CC(=O)N1CCC(c2c[nH]c3ncnc(Nc4ccc(OCc5nc(F)c(C)s5)c(Cl)c4)c23)CC1] | 1-(4-(4-((3-chloro-4-((4-fluoro-5- methylthiazol-2-yl)methoxy)phenyl)amino)- 7H-pyrrolo[2,3-d]pyrimidin-5-yl)piperidin-1- yl)prop-2-en-1-one |
| 213 | 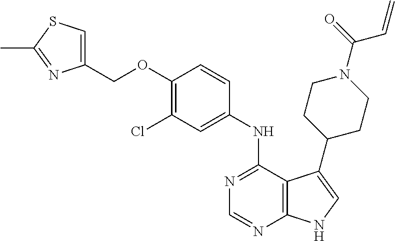 [SMILES: C=CC(=O)N1CCC(c2c[nH]c3ncnc(Nc4ccc(OCc5csc(C)n5)c(Cl)c4)c23)CC1] | 1-(4-(4-((3-chloro-4-((2-methylthiazol-4- yl)methoxy)phenyl)amino)-7H-pyrrolo[2,3- d]pyrimidin-5-yl)piperidin-1-yl)prop-2-en-1- one |
| 214 | 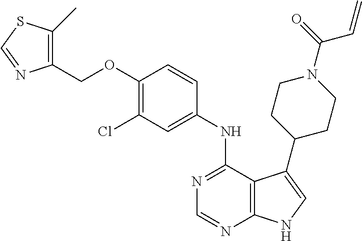 [SMILES: C=CC(=O)N1CCC(c2c[nH]c3ncnc(Nc4ccc(OCc5ncsc5C)c(Cl)c4)c23)CC1] | 1-(4-(4-((3-chloro-4-((5-methylthiazol-4- yl)methoxy)phenyl)amino)-7H-pyrrolo[2,3- d]pyrimidin-5-yl)piperidin-1-yl)prop-2-en-1- one |
| 215 | 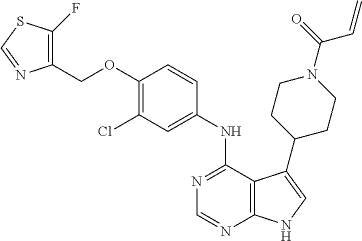 [SMILES: C=CC(=O)N1CCC(c2c[nH]c3ncnc(Nc4ccc(OCc5ncsc5F)c(Cl)c4)c23)CC1] | 1-(4-(4-((3-chloro-4-((5-fluorothiazol-4- yl)methoxy)phenyl)amino)-7H-pyrrolo[2,3- d]pyrimidin-5-yl)piperidin-1-yl)prop-2-en-1- one |
| 216 | 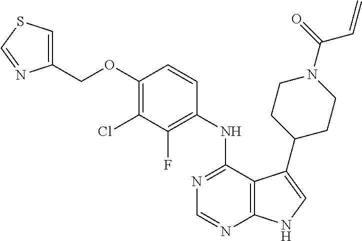 [SMILES: C=CC(=O)N1CCC(c2c[nH]c3ncnc(Nc4ccc(OCc5cscn5)c(Cl)c4F)c23)CC1] | 1-(4-(4-((3-chloro-2-fluoro-4-(thiazol-4- ylmethoxy)phenyl)amino)-7H-pyrrolo[2,3- d]pyrimidin-5-yl)piperidin-1-yl)prop-2-en-1- one |
| 217 | 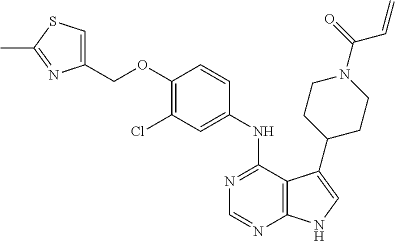 [SMILES: C=CC(=O)N1CCC(c2c[nH]c3ncnc(Nc4ccc(OCc5csc(C)n5)c(Cl)c4)c23)CC1] | 1-(4-(4-((3-chloro-4-((2-methylthiazol-4- yl)methoxy)phenyl)amino)-7H-pyrrolo[2,3- d]pyrimidin-5-yl)piperidin-1-yl)prop-2-en-1- one |
| 218 | 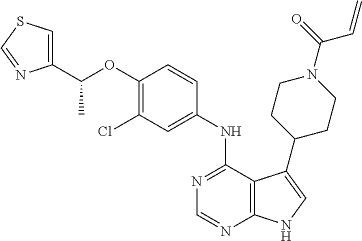 [SMILES: C=CC(=O)N1CCC(c2c[nH]c3ncnc(Nc4ccc(O[C@H](C)c5cscn5)c(Cl)c4)c23)CC1] | (R)-1-(4-(4-((3-chloro-4-(1-(thiazol-4- yl)ethoxy)phenyl)amino)-7H-pyrrolo[2,3- d]pyrimidin-5-yl)piperidin-1-yl)prop-2-en-1- one |
| 219 | 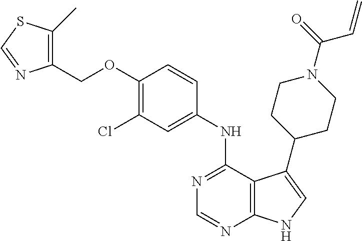 [SMILES: C=CC(=O)N1CCC(c2c[nH]c3ncnc(Nc4ccc(OCc5ncsc5C)c(Cl)c4)c23)CC1] | 1-(4-(4-((3-chloro-4-((5-methylthiazol-4- yl)methoxy)phenyl)amino)-7H-pyrrolo[2,3- d]pyrimidin-5-yl)piperidin-1-yl)prop-2-en-1- one |
| 220 | 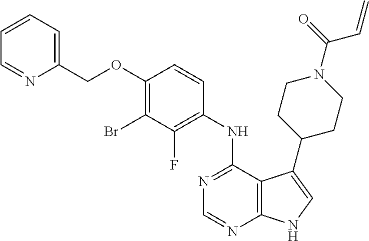 [SMILES: C=CC(=O)N1CCC(c2c[nH]c3ncnc(Nc4ccc(OCc5ccccn5)c(Br)c4F)c23)CC1] | 1-(4-(4-((3-bromo-2-fluoro-4-(pyridin-2- ylmethoxy)phenyl)amino)-7H-pyrrolo[2,3- d]pyrimidin-5-yl)piperidin-1-yl)prop-2-en-1- one |
| 221 | 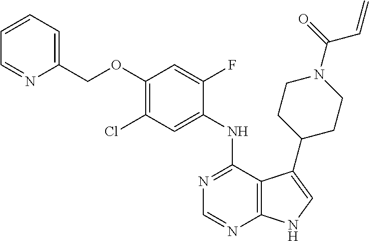 [SMILES: C=CC(=O)N1CCC(c2c[nH]c3ncnc(Nc4cc(Cl)c(OCc5ccccn5)cc4F)c23)CC1] | 1-(4-(4-((5-chloro-2-fluoro-4-(pyridin-2- ylmethoxy)phenyl)amino)-7H-pyrrolo[2,3- d]pyrimidin-5-yl)piperidin-1-yl)prop-2-en-1- one |
| 222 | 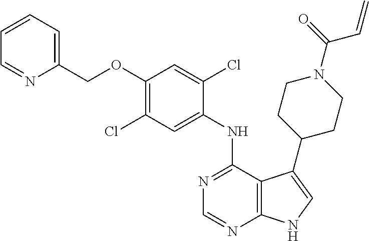 [SMILES: C=CC(=O)N1CCC(c2c[nH]c3ncnc(Nc4cc(Cl)c(OCc5ccccn5)cc4Cl)c23)CC1] | 1-(4-(4-((2,5-dichloro-4-(pyridin-2- ylmethoxy)phenyl)amino)-7H-pyrrolo[2,3- d]pyrimidin-5-yl)piperidin-1-yl)prop-2-en-1- one |
| 223 | 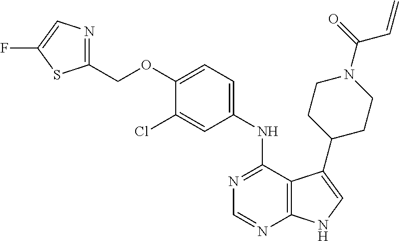 [SMILES: C=CC(=O)N1CCC(c2c[nH]c3ncnc(Nc4ccc(OCc5ncc(F)s5)c(Cl)c4)c23)CC1] | 1-(4-(4-((3-chloro-4-((5-fluorothiazol-2- yl)methoxy)phenyl)amino)-7H-pyrrolo[2,3- d]pyrimidin-5-yl)piperidin-1-yl)prop-2-en-1- one |
| 224 | 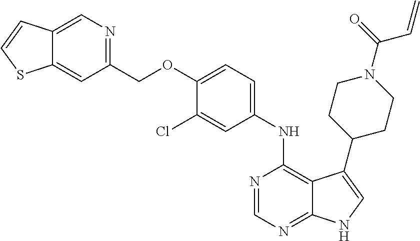 [SMILES: C=CC(=O)N1CCC(c2c[nH]c3ncnc(Nc4ccc(OCc5cc6sccc6cn5)c(Cl)c4)c23)CC1] | 1-(4-(4-((3-chloro-4-(thieno[3,2-c]pyridin-6- ylmethoxy)phenyl)amino)-7H-pyrrolo[2,3- d]pyrimidin-5-yl)piperidin-1-yl)prop-2-en-1- one |
| 225 | 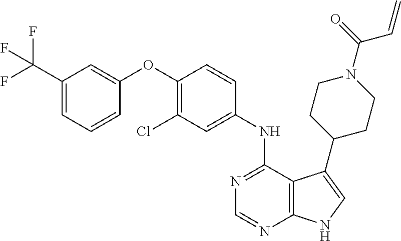 [SMILES: C=CC(=O)N1CCC(c2c[nH]c3ncnc(Nc4ccc(Oc5cccc(C(F)(F)F)c5)c(Cl)c4)c23)CC1] | 1-(4-(4-((3-chloro-4-(3- (trifluoromethyl)phenoxy)phenyl)amino)-7H- pyrrolo[2,3-d]pyrimidin-5-yl)piperidin-1- yl)prop-2-en-1-one |
| 226 | 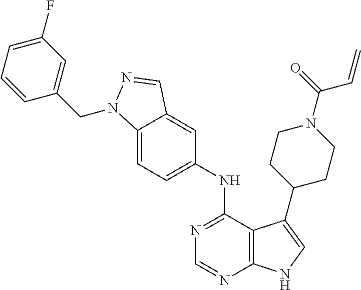 [SMILES: C=CC(=O)N1CCC(c2c[nH]c3ncnc(Nc4ccc5c(cnn5Cc5cccc(F)c5)c4)c23)CC1] | 1-(4-(4-((1-(3-fluorobenzyl)-1H-indazol-5- yl)amino)-7H-pyrrolo[2,3-d]pyrimidin-5- yl)piperidin-1-yl)prop-2-en-1-one |
| 227 | 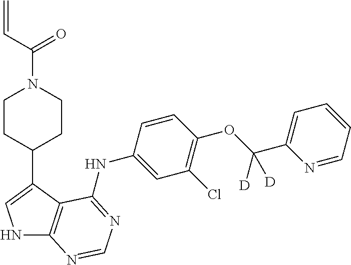 [SMILES: [2H]C([2H])(Oc1ccc(Nc2ncnc3[nH]cc(C4CCN(C(=O)C=C)CC4)c23)cc1Cl)c1ccccn1] | 1-(4-(4-((3-chloro-4-(pyridin-2-ylmethoxy- d2)phenyl)amino)-7H-pyrrolo[2,3- d]pyrimidin-5-yl)piperidin-1-yl)prop-2-en-1- one |
| 228 | 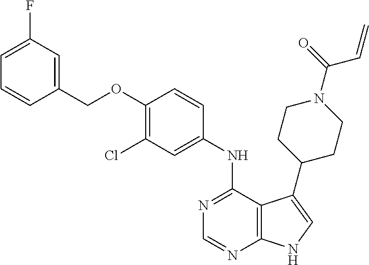 [SMILES: C=CC(=O)N1CCC(c2c[nH]c3ncnc(Nc4ccc(OCc5cccc(F)c5)c(Cl)c4)c23)CC1] | 1-(4-(4-((3-chloro-4-((3- fluorobenzyl)oxy)phenyl)amino)-7H- pyrrolo[2,3-d]pyrimidin-5-yl)piperidin-1- yl)prop-2-en-1-one |
| 229 | 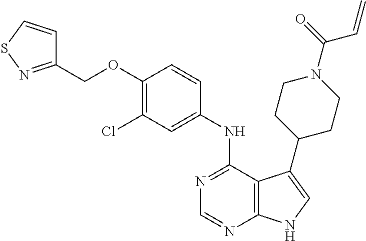 [SMILES: C=CC(=O)N1CCC(c2c[nH]c3ncnc(Nc4ccc(OCc5ccsn5)c(Cl)c4)c23)CC1] | 1-(4-(4-((3-chloro-4-(isothiazol-3- ylmethoxy)phenyl)amino)-7H-pyrrolo[2,3- d]pyrimidin-5-yl)piperidin-1-yl)prop-2-en-1- one |
| 230 | 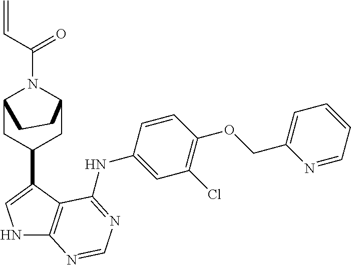 | 1-((1R,3r,5S)-3-(4-((3-chloro-4-(pyridin-2- ylmethoxy)phenyl)amino)-7H-pyrrolo[2,3- d]pyrimidin-5-yl)-8-azabicyclo[3.2.1]octan-8- yl)prop-2-en-1-one |
| 231 | 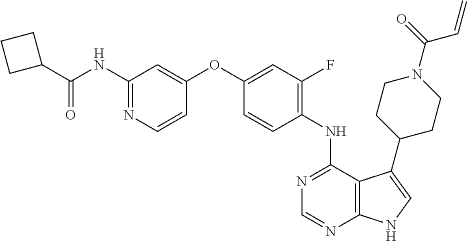 [SMILES: C=CC(=O)N1CCC(c2c[nH]c3ncnc(Nc4ccc(Oc5ccnc(NC(=O)C6CCC6)c5)cc4F)c23)CC1] | N-(4-(4-((5-(1-acryloylpiperidin-4-yl)-7H- pyrrolo[2,3-d]pyrimidin-4-yl)amino)-3- fluorophenoxy)pyridin-2- yl)cyclobutanecarboxamide |
| 232 | 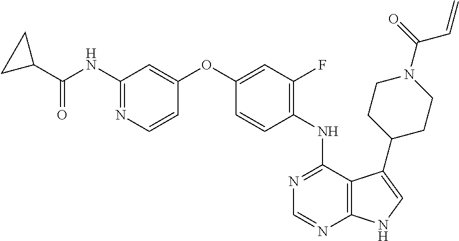 [SMILES: C=CC(=O)N1CCC(c2c[nH]c3ncnc(Nc4ccc(Oc5ccnc(NC(=O)C6CC6)c5)cc4F)c23)CC1] | N-(4-(4-((5-(1-acryloylpiperidin-4-yl)-7H- pyrrolo[2,3-d]pyrimidin-4-yl)amino)-3- fluorophenoxy)pyridin-2- yl)cyclopropanecarboxamide |
| 233 | 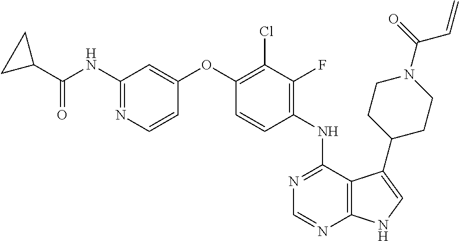 [SMILES: C=CC(=O)N1CCC(c2c[nH]c3ncnc(Nc4ccc(Oc5ccnc(NC(=O)C6CC6)c5)c(Cl)c4F)c23)CC1] | N-(4-(4-((5-(1-acryloylpiperidin-4-yl)-7H- pyrrolo[2,3-d]pyrimidin-4-yl)amino)-2-chloro- 3-fluorophenoxy)pyridin-2- yl)cyclopropanecarboxamide |
| 234 | 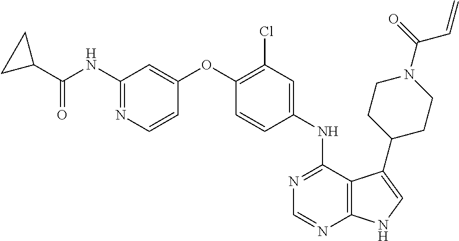 [SMILES: C=CC(=O)N1CCC(c2c[nH]c3ncnc(Nc4ccc(Oc5ccnc(NC(=O)C6CC6)c5)c(Cl)c4)c23)CC1] | N-(4-(4-((5-(1-acryloylpiperidin-4-yl)-7H- pyrrolo[2,3-d]pyrimidin-4-yl)amino)-2- chlorophenoxy)pyridin-2- yl)cyclopropanecarboxamide |
| 235 | 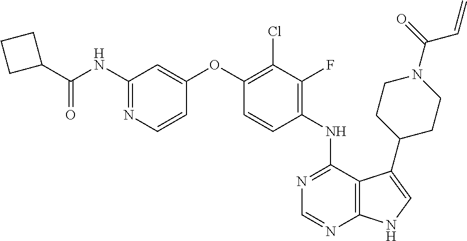 [SMILES: C=CC(=O)N1CCC(c2c[nH]c3ncnc(Nc4ccc(Oc5ccnc(NC(=O)C6CCC6)c5)c(Cl)c4F)c23)CC1] | N-(4-(4-((5-(1-acryloylpiperidin-4-yl)-7H- pyrrolo[2,3-d]pyrimidin-4-yl)amino)-2-chloro- 3-fluorophenoxy)pyridin-2- yl)cyclobutanecarboxamide |
| 236 | 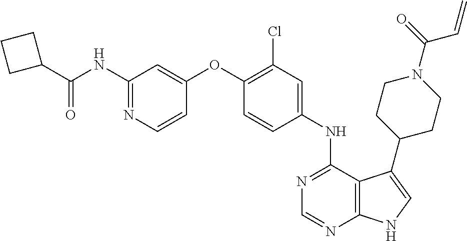 [SMILES: C=CC(=O)N1CCC(c2c[nH]c3ncnc(Nc4ccc(Oc5ccnc(NC(=O)C6CCC6)c5)c(Cl)c4)c23)CC1] | N-(4-(4-((5-(1-acryloylpiperidin-4-yl)-7H- pyrrolo[2,3-d]pyrimidin-4-yl)amino)-2- chlorophenoxy)pyridin-2- yl)cyclobutanecarboxamide |
| 237 | 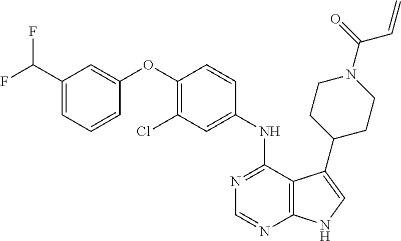 [SMILES: C=CC(=O)N1CCC(c2c[nH]c3ncnc(Nc4ccc(Oc5cccc(C(F)F)c5)c(Cl)c4)c23)CC1] | 1-(4-(4-((3-chloro-4-(3- (difluoromethyl)phenoxy)phenyl)amino)-7H- pyrrolo[2,3-d]pyrimidin-5-yl)piperidin-1- yl)prop-2-en-1-one |

### Pharmaceutical Compositions

Pharmaceutical compositions of the present disclosure comprise at least one compound of Formula (1), or tautomer, stereoisomer or a mixture of stereoisomers, or a pharmaceutically acceptable salt, or hydrate, or deuterated derivative thereof formulated together with one or more pharmaceutically acceptable carriers. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (1), or a tautomer, stereoisomer or a mixture of stereoisomers, or a pharmaceutically acceptable salt, or hydrate, or deuterated derivative thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (1), or a tautomer, stereoisomer or a mixture of stereoisomers, or a pharmaceutically acceptable salt, or hydrate, or deuterated derivative thereof) is generally present at a concentration of from about 0.1% to about 15% w/w of the composition, for example, from about 0.5 to about 2%.

The amount of active compound administered may be dependent on the subject being treated, the subjects weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

The present disclosure provides a compound of Formula (1), or a tautomer, stereoisomer or a mixture of stereoisomers, or a pharmaceutically acceptable salt, or hydrate, or deuterated derivative thereof, to be administered to treat cancer in a subject in need thereof.

Disclosed are methods of treating cancer in a subject in need thereof, comprising administering to the subject an effective amount of a compound disclosed herein or of the pharmaceutical composition disclosed herein.

In some embodiments, the cancer is associated with an EGFR mutation.

In some embodiments, the cancer is chosen from breast cancer, lung cancer, pancreatic cancer, colon cancer, head and neck cancer, renal call carcinoma, squamous cell carcinoma, thyroid cancer, gall bladder cancer, thyroid cancer, bile duct cancer, ovarian cancer, endometrial cancer, prostate cancer, and esophageal cancer. In some embodiments, the cancer is lung cancer. In some embodiments, the cancer is non-small cell lung cancer. In some embodiments, the cancer is pancreatic cancer. In some embodiments, the cancer is colon cancer. In some embodiments, the cancer is breast cancer. In some embodiments, the cancer is head and neck cancer. In some embodiments, the cancer is sinonasal squamous cell carcinoma.

Disclosed are uses of the disclosed compounds or of the disclosed pharmaceutical compositions, in the preparation of a medicament.

Also disclosed herein is a method of treating cancer, in a subject in need thereof, comprising administering to said subject a compound of Formula (1) (e.g. Formula (2), Formula (3), Formula (4), Formula (5)), or a pharmaceutically acceptable salt thereof, or a pharmaceutical composition comprising a compound of Formula (1) or a pharmaceutically acceptable salt thereof.

In some embodiments, the method further comprises administering to the subject a compound of Formula (1) or a pharmaceutical composition comprising a compound of Formula (1) in combination with another agent. In some embodiments, the agent is chosen from gemcitabine, cisplatin, erlotinib, gefitinib, pemetrexed, bevacizumab, cetuximab, trastuzumab, pertuzumab, sorafenib, lapatinib, cobimetinib, selumetinib, and everolimus. In some embodiments, the disclosed compounds or the disclosed pharmaceutical compositions and the additional agent are administered concomitantly. In some embodiments, the disclosed compounds or the disclosed pharmaceutical compositions and the additional agent are administered sequentially.

In some embodiments, the cancer is associated with an EGFR or HER2 exon 20 insertion mutation.

In some embodiments, a compound of Formula (1), or a tautomer, stereoisomer or a mixture of stereoisomers, or a pharmaceutically acceptable salt, or hydrate, or deuterated derivative thereof, is administered as a pharmaceutical composition.

The concentration and route of administration to the patient will vary depending on the cancer to be treated.

In one embodiment, a compound of Formula (1), or a tautomer, stereoisomer or a mixture of stereoisomers, or a pharmaceutically acceptable salt, or hydrate, or deuterated derivative thereof, is administered in combination with another therapeutic agent, e.g., chemotherapy, or used in combination with other treatments, such as radiation or surgical intervention, either as an adjuvant prior to surgery or post-operatively.

Also provided herein is a compound of Formula (1), or a tautomer, stereoisomer or a mixture of stereoisomers, or a pharmaceutically acceptable salt, or hydrate, or deuterated derivative thereof, or a pharmaceutical composition thereof as defined herein for use in therapy.

Also provided herein is a compound of Formula (1), or a tautomer, stereoisomer or a mixture of stereoisomers, or a pharmaceutically acceptable salt, or hydrate, or deuterated derivative thereof, or a pharmaceutical composition thereof as defined herein for use in the treatment of cancer.

Also provided herein is a compound of Formula (1), or a tautomer, stereoisomer or a mixture of stereoisomers, or a pharmaceutically acceptable salt, or hydrate, or deuterated derivative thereof, for use in the inhibition of EGFR.

Also provided herein is a compound of Formula (1), or a tautomer, stereoisomer or a mixture of stereoisomers, or a pharmaceutically acceptable salt, or hydrate, or deuterated derivative thereof, or a pharmaceutical composition thereof as defined herein, for use in the treatment of a disease or disorder associated with an EGFR or HER2 exon 20 insertion mutation.

Also provided herein is the use of a compound of Formula (1), or a tautomer, stereoisomer or a mixture of stereoisomers, or a pharmaceutically acceptable salt, or hydrate, or deuterated derivative thereof, as defined herein in the manufacture of a medicament for the treatment of cancer.

Also provided herein is a use of a compound of Formula (1), or a tautomer, stereoisomer or a mixture of stereoisomers, or a pharmaceutically acceptable salt, or hydrate, or deuterated derivative thereof, as defined herein in the manufacture of a medicament for the inhibition of activity of EGFR.

Also provided herein is the use of a compound of Formula (1), or a tautomer, stereoisomer or a mixture of stereoisomers, or a pharmaceutically acceptable salt, or hydrate, or deuterated derivative thereof, as defined herein, in the manufacture of a medicament for the treatment of a disease or disorder associated with an EGFR or HER2 exon 20 insertion mutation.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The invention also provides for a method of inhibiting EGFR kinase activity in a cell comprising contacting the cell with an effective amount of an EGFR antagonist. In one embodiment, the administered amount is a therapeutically effective amount and the inhibition of EGFR kinase activity further results in the inhibition of the growth of the cell. In a further embodiment, the cell is a cancer cell.

Inhibition of cell proliferation is measured using methods known to those skilled in the art. For example, a convenient assay for measuring cell proliferation is the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high-throughput screening (HTS). See Cree et al (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera Imaging device. The luminescence output is expressed as relative light units (RLU). Inhibition of cell proliferation may also be measured using colony formation assays known in the art.

Furthermore, the invention provides for methods of treating a condition associated with an EGFR or HER2 exon 20 insertion mutation in a subject suffering therefrom, comprising administering to the subject a therapeutically effective amount of an EGFR antagonist. In one embodiment, the condition is a cell proliferative disease.

Treatment of the cell proliferative disorder by administration of an EGFR antagonist results in an observable and/or measurable reduction in or absence of one or more of the following: reduction in the number of cancer cells or absence of the cancer cells; reduction in the tumor size; inhibition of cancer cell infiltration into peripheral organs including the spread of cancer into soft tissue and bone; inhibition of tumor metastasis; inhibition, to some extent, of tumor growth; and/or relief to some extent, one or more of the symptoms associated with the specific cancer; reduced morbidity and mortality, and improvement in quality of life issues. To the extent the EGFR antagonist may prevent growth and/or kill existing cancer cells, it may be cytostatic and/or cytotoxic. Reduction of these signs or symptoms may also be felt by the patient.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −10° C. to about 200° C. Further, except as otherwise specified, reaction times and conditions are intended to be approximate, e.g., taking place at about atmospheric pressure within a temperature range of about −10° C. to about 200° C. over a period that can be, for example, about 1 to about 24 hours; reactions left to run overnight in some embodiments can average a period of about 16 hours.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3rd Ed., 1990 New York: Plenum Press; Mundy et al., Name Reaction and Reagents in Organic Synthesis, 2nd Ed., 2005 Hoboken, NJ: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3rd Ed., John Wiley & Sons). These groups may be removed at a convenient stage of the compound synthesis using methods that are readily apparent to those skilled in the art.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

The following abbreviations have the definitions set forth below:
Ac Acetyl
ACN acetonitrile
Boc tert-Butyloxycarbonyl
dba dibenzylideneacetone
DCM dichloromethane
DIEA diisopropylethylamine
DMF dimethylformamide
dppf 1,1′-Bis(diphenyphosphino)ferrocene
EA ethyl acetate
eq equivalent
FA formic acid
h hour
IPA isopropyl alcohol
LC-MS liquid chromatography mass spectrometry
LDA Lithium diisopropylamide
MW Microwave
NMR nuclear magnetic resonance
NMP N-Methyl-2-pyrrolidone
PE petroleum ether
Prep-TLC preparative thin layer chromatography
TLC thin layer chromatography
tol toluene
THF tetrahydrofuran
TsOH p-toluenesulfonic acid

### General Synthetic Schemes

The claimed compounds can be prepared according to the following schemes. The following schemes represent the general methods used in preparing these compounds. However, the synthesis of these compounds is not limited to these representative methods, as they can also be prepared through various other methods by those skilled in the art of synthetic chemistry.

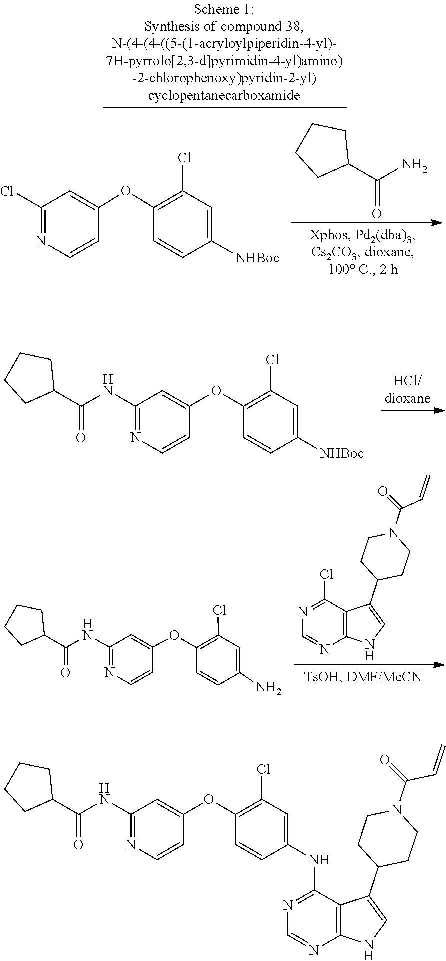 [SMILES: CC(C)(C)OC(=O)Nc1ccc(Oc2ccnc(Cl)c2)c(Cl)c1 ; NC(=O)C1CCCC1 ; CC(C)(C)OC(=O)Nc1ccc(Oc2ccnc(NC(=O)C3CCCC3)c2)c(Cl)c1 ; Nc1ccc(Oc2ccnc(NC(=O)C3CCCC3)c2)c(Cl)c1 ; C=CC(=O)N1CCC(c2c[nH]c3ncnc(Cl)c23)CC1 ; C=CC(=O)N1CCC(c2c[nH]c3ncnc(Nc4ccc(Oc5ccnc(NC(=O)C6CCCC6)c5)c(Cl)c4)c23)CC1]

Compounds 90, 91, 182, 192, 193, 194, and 196 can be prepared via a similar procedure to the one shown in Scheme 1.

TABLE 2
Characterization of compounds

| Compound No. | LC-MS | ¹H NMR |
|---|---|---|
| 38 | [M + H]⁺ = 586.2 | ¹H NMR (DMSO-d₆, 400 MHz) δ 11.71 (s, 1H), |
| | | 10.51 (s, 1H), 8.31-8.28 (m, 1H), 8.24 (s, 1H), |
| | | 8.20 (d, J = 6.0 Hz, 1H), 8.13.-8.11 (m, 1H), 7.81- |
| | | 7.77 (m, 1H), 7.67 (d, J = 2.4 Hz, 1H), 7.36 (d, J = |
| | | 8.8 Hz, 1H), 7.11 (d, J = 2.0 Hz, 1H), 6.84 (dd, J = |
| | | 10.4, 16.4 Hz, 1H), 6.69 (dd, J = 2.4, 5.6 Hz, 1H), |
| | | 6.10 (dd, J = 2.0, 16.4 Hz, 1H), 5.68-5.64 (m, 1H), |
| | | 4.58 (d, J = 12.8 Hz, 1H), 4.17 (d, J = 13.2 Hz, |
| | | 1H), 3.62-3.55 (m, 1H), 3.27-3.24 (m, 1H), 2.92- |
| | | 2.83 (m, 2H), 2.07-2.01 (m, 2H), 1.83-1.77 (m, |
| | | 2H), 1.65-1.58 (m, 4H), 1.50-1.40 (m, 4H) |
| 90 | [M + H]⁺ = 570.3 | ¹H NMR (DMSO₆, 400 MHz) δ 11.63 (s, 1 H), |
| | | 10.53 (s, 1 H), 8.23 (d, J = 5.6 Hz, 1 H), 8.18-8.12 |
| | | (m, 1 H), 8.02-7.93 (m, 2 H), 7.76 (d, J = 2.0 Hz, |
| | | 1 H), 7.28 (dd, J = 11.2, 2.4 Hz, 1 H), 7.14-7.05 |
| | | (m, 2 H), 6.85 (dd, J = 16.4, 10.4 Hz, 1 H), 6.76 |
| | | (dd, J = 5.6, 2.4 Hz, 1 H), 6.11 (dd, J = 16.4, 2.4 |
| | | Hz, 1 H), 5.67 (dd, J = 10.4, 2.4 Hz, 1 H), 4.59 (d, J = |
| | | 11.2 Hz, 1 H), 4.27-4.12 (m, 1 H), 3.51-3.41 |
| | | (m, 1 H), 3.27-3.20 (m, 1 H), 2.95-2.78 (m, 2 H), |
| | | 2.16-2.03 (m, 2 H), 1.87-1.75 (m, 2 H), 1.70- |
| | | 1.57 (m, 4 H), 1.57-1.42 (m, 4 H) |
| 91 | [M + H]⁺ = 558.3 | ¹H NMR (CDCl₃, 400 MHz) δ 9.15-9.00 (m, 1H), |
| | | 8.98-8.84 (m, 1H), 8.49 (s, 1H), 8.15 (d, J = 5.6 |
| | | Hz, 1H), 8.04 (s, 1H), 7.88 (d, J = 2.0 Hz, 1H), 7.19 |
| | | (d, J = 3.6 Hz, 1H), 7.02-6.96 (m, 2H), 6.92 (d, J = |
| | | 1.6 Hz, 1H), 6.75-6.54 (m, 2H), 6.40-6.24 (m, |
| | | 1H), 5.78-5.67 (m, 1H), 5.00-4.88 (m, 1H), 4.28- |
| | | 4.16 (m, 1H), 3.41-3.26 (m, 1H), 3.19-3.04 (m, |
| | | 1H), 2.95-2.81 (m, 1H), 2.32-2.20 (m, 2H), 1.81- |
| | | 1.70 (m, 2H), 1.31 (s, 9H) |
| 182 | [M + H]⁺ = 545.3 | ¹H NMR (DMSO₆, 400 MHz) δ 13.38-13.35 (m, |
| | | 1H), 10.54 (s, 1H), 8.68 (s, 1H), 8.25 (d, J = 5.2 |
| | | Hz, 2H), 7.78 (d, J = 2.0 Hz, 1H), 7.74 (t, J = 8.4 |
| | | Hz, 1H), 7.30 (d, J = 10.4 Hz, 1H), 7.13 (d, J = |
| | | 10.4 Hz, 1H), 6.86-6.83 (m, 1H), 6.79 (dd, J = 2.4, |
| | | 5.6 Hz, 1H), 6.12 (dd, J = 2.4, 16.8 Hz, 1H), 5.68 |
| | | (dd, J = 2.4, 10.4 Hz, 1H), 4.52 (d, J = 12.0 Hz, |
| | | 1H), 4.17 (d, J = 12.8 Hz, 1H), 3.76-3.71 (m, 1H), |
| | | 2.96-2.93 (m, 1H), 2.77-2.70 (m, 2H), 2.08 (d, J = |
| | | 10.4 Hz, 2H), 1.70-1.65 (m, 2H), 1.05 (d, J = 6.8 |
| | | Hz, 6H) |
| 193 | [M + H]⁺ = 571.3 | ¹H NMR (DMSO₆, 400 MHz) δ 13.39-13.19 (m, |
| | | 1H), 10.52 (s, 1H), 8.73-8.65 (m, 1H), 8.24-8.20 |
| | | (m, 2H), 7.77 (d, J = 2.4 Hz, 1H), 7.70-7.69 (m, |
| | | 1H), 7.27 (d, J = 10.0 Hz, 1H), 7.08 (d, J = 7.6 Hz, |
| | | 1H), 6.89-6.82 (m, 1H), 6.76 (dd, J = 2.4, 3.2 Hz, |
| | | 1H), 6.12 (dd, J = 2.4, 16.8 Hz, 1H), 5.67 (dd, J = |
| | | 2.4, 12.8 Hz, 1H), 4.51 (d, J = 3.6 Hz, 1H), 4.16 (d, |
| | | J = 12.8 Hz, 1H), 3.73-3.69 (m, 1H), 3.19-3.16 (m, |
| | | 1H), 2.95-2.89 (m, 2H), 2.10-2.05 (s, 2H), 1.83- |
| | | 1.81 (m, 2H), 1.67-1.60 (m, 6H), 1.51-1.50 (m, 2H) |
| 194 | [M + H]⁺ = 587.3 | ¹H NMR (CD₃CN, 400 MHz) δ 8.65 ( s, 1H), 8.33 |
| | | (s, 1H), 8.18 (d, J = 5.6 Hz, 1H), 8.11 (t, J = 8.8 |
| | | Hz, 1H), 7.77 (d, J = 2.4 Hz, 1H), 7.38 (s, 1H), |
| | | 7.14-7.04 (m, 2H), 6.80-6.70 (m, 2H), 6.14 (dd, J = |
| | | 2.4, 16.8 Hz, 1H), 5.65 (dd, J = 2.4, 10.4 Hz, 1H), |
| | | 4.65-4.59 (m, 1H), 4.22-4.14 (m, 1H), 3.94-3.89 |
| | | (m, 2H), 3.47-3.34 (m, 4H), 2.99-2.92 (m, 1H), |
| | | 2.67-2.59 (m, 1H), 2.19-2.13 (m, 2H), 1.80-1.64 |
| | | (m, 6H) |

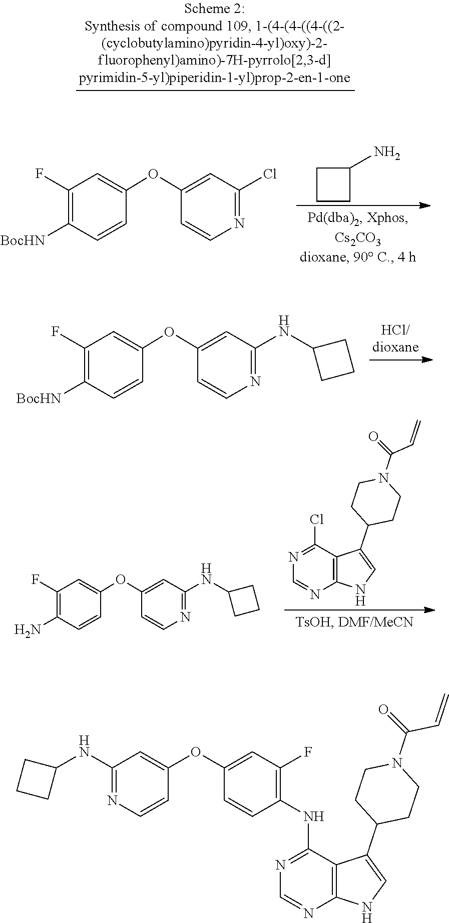

Compounds 40, 43, 56, 57, 58, 63, 65, 72, 78, 83, 84, 107, 127, 129, 130, 131, 135, 139, 140, 141, 142, 1, 145, 146, 147, 148, 172, 173, 174, 175, 176, 177, 178, 179, 180, and 181 can be prepared via a similar procedure to the one shown in Scheme 2.

TABLE 3
Characterization of compounds

| Compound | | |
|---|---|---|
| No. | LC-MS | ¹H NMR |
| 40 | [M + H]⁺ = 585.2 | ¹H NMR (DMSO-d₆, 400 MHz) δ 11.60 |
| | | (s, 1H), 8.59 (d, J = 4.4 Hz, 1H), 8.19- |
| | | 8.16 (m, 1H), 8.04-7.93 (m, 2H), 7.88 |
| | | (td, J = 1.6, 7.6 Hz, 1H), 7.62-7.56 (m, |
| | | 2H), 7.36 (dd, J = 5.2, 7.2 Hz, 1H), 7.19 |
| | | (d, J = 8.8 Hz, 1H), 7.04 (s, 1H), 6.83 |
| | | (dd, J = 10.4, 16.8 Hz, 1H), 6.10 (dd, J = |
| | | 2.4, 16.8, Hz, 1H), 5.66 (dd, J = 2.4, |
| | | 10.4 Hz, 1H), 5.28 (s, 2H), 4.56 (m, |
| | | 1H), 4.20-4.10 (m, 1H), 3.51 (m, 1H), |
| | | 3.30-3.21 (m, 1H), 2.83 (m, 1H), 2.03 |
| | | (m, 2H), 1.49-1.37 (m, 2H) |
| 43 | [M + H]⁺ = 612.2. | ¹H NMR (DMSO-d₆, 400 MHz) δ 11.70 |
| | | (br, 1H), 8.27 (s, 1H) 8.20 (s, 1H), 8.11- |
| | | 8.07 (m, 1H), 7.99 (d, J = 5.8 Hz, 1H), |
| | | 7.79-7.72 (m, 1H), 7.30 (d, J = 8.8 Hz, |
| | | 1H), 7.11 (s, 1H) 6.88-6.77 (m, 1H), |
| | | 6.13-6.07 (m, 2H), 6.01 (d, J = 2.0 Hz, |
| | | 1H), 5.67 (dd, J = 2.4, 10.4 Hz, 1H), |
| | | 4.62-4.52 (m, 1H), 4.22-4.11 (m, 1H), |
| | | 3.67 (dd, J = 8.4, 10.8 Hz, 1H), 3.62- |
| | | 3.54 (m, 1H), 3.46 (dd, J = 6.4, 10.8 Hz, |
| | | 2H), 3.41-3.34 (m, 2H), 3.26 (br, 1H), |
| | | 2.91-2.79 (m, 1H), 2.26 (dd, J = 5.6, |
| | | 13.2 Hz, 1H), 2.10-2.02 (m, 3H), 1.52- |
| | | 1.41 (m, 2H) |
| 49 | [M + H]⁺ = 567.2 | ¹H NMR (CD₃OD, 400 MHz) δ 8.24 (s, |
| | | 2H) 8.16 (d, J = 6.36 Hz, 1H) 8.00 (d, J = |
| | | 2.40 Hz, 1H) 7.75-7.81 (m, 1H) 7.65 |
| | | (dd, J = 9.00, 2.08 Hz, 1H) 7.29-7.35 |
| | | (m, 2H) 7.06 (s, 1H) 7.00-7.05 (m, 1H) |
| | | 6.89-6.96 (m, 1H) 6.80 (dd, J = |
| | | 16.40, 10.40 Hz, 1H) 6.67-6.75 (m, 1H) |
| | | 6.15-6.22 (m, 1H) 5.73 (m, 1H) |
| | | 4.66-4.72 (m, 1H) 4.18-4.32 (m, 1H) |
| | | 3.44-3.50 (m, 1H) 3.33-3.40 (m, 1H) |
| | | 2.91-2.99 (m, 1H) 2.15-2.24 (m, 2H) |
| | | 1.55-1.66 (m, 2H) |
| 56 | [M + H]⁺ = 528.1 | ¹H NMR (DMSO-d₆, 400 MHz) δ 11.63 |
| | | (s, 1H), 8.19-8.11 (m, 1H), 7.99-7.85 |
| | | (m, 3H), 7.22 (dd, J = 2.4, 11.2 Hz, 1H), |
| | | 7.10-7.01 (m, 2H), 6.98-6.90 (m, 1H), |
| | | 6.86 (dd, J = 10.4, 16.8 Hz, 1H), 6.21- |
| | | 6.15 (m, 1H), 6.15-6.08 (m, 2H), 4.73- |
| | | 4.45 (m, 1H), 4.30-4.06 (m, 1H), 3.51- |
| | | 3.40 (m, 1H), 3.29-3.22 (m, 1H), 2.92- |
| | | 2.76 (m, 1H), 2.20-2.00 (m, 2H), 1.59- |
| | | 1.41 (m, 2H), 1.29 (s, 2H), 0.72-0.53 |
| | | (m, 4H) |
| 57 | [M + H]⁺ = 560.3 | ¹H NMR (DMSO-d₆, 400 MHz) δ 11.61 |
| | | (s, 1H), 8.16-8.12 (m, 1H), 8.03 (d, J = |
| | | 2.8 Hz, 1H), 7.93-7.85 (m, 2H), 7.21- |
| | | 7.16 (m, 1H), 7.07-6.99 (m, 2H), 6.88- |
| | | 6.79 (m, 1H), 6.24 (dd, J = 2.0, 5.6 Hz, |
| | | 1H), 6.14-6.07 (m, 1H), 6.04-5.99 (m, |
| | | 1H), 5.71-5.56 (m, 1H), 4.62-4.55 (m, |
| | | 1H), 4.22-4.11 (m, 1H), 3.73-3.64 (m, |
| | | 1H), 3.63-3.54 (m, 1H), 3.49-3.36 (m, |
| | | 4H), 2.86-2.77 (m, 1H), 2.25-2.15 (m, |
| | | 2H), 2.12-2.01 (m, 2H), 1.52 (d, J = 8.8 |
| | | Hz, 3H), 1.50-1.43 (m, 2H) |
| 58 | [M + H]⁺ = 556.2 | ¹H NMR (CD₃OD, 400 MHz) δ 8.18 (s, |
| | | 1H), 8.15-8.05 (m, 1H), 7.92 (d, J = 6.0 |
| | | Hz, 1H), 7.09-7.04 (m, 2H), 7.01 (d, J = |
| | | 9.2 Hz, 1H), 6.82 (dd, J = 10.8, 16.8 Hz, |
| | | 1H), 6.37 (dd, J = 2.0, 6.0 Hz, 1H), 6.25- |
| | | 6.17 (m, 2H), 5.75 (dd, J = 2.0, 10.8 |
| | | Hz, 1H), 4.77-4.68 (m, 1H), 4.27 (d, J = |
| | | 13.6 Hz, 1H), 4.10-4.02 (m, 2H), 3.38 (s, |
| | | 2H), 3.02-2.91 (m, 1H), 2.30-2.20 (m, |
| | | 2H), 1.72-1.58 (m, 2H) |
| 63 | [M + H]⁺ = 568.3 | ¹H NMR (DMSO-d₆, 400 MHz) δ 11.63 |
| | | (s, 1H), 8.15 (s, 1H), 8.00 (d, J = 5.6 Hz, |
| | | 1H), 7.96-7.83 (m, 2H), 7.23-7.14 (m, |
| | | 1H), 7.07 (s, 1H), 7.05-6.98 (m, 1H), |
| | | 6.93-6.80 (m, 1H), 6.24-6.17 (m, 1H), |
| | | 6.15-6.07 (m, 1H), 6.02 (d, J = 2.0 Hz, |
| | | 1H), 5.76-5.55 (m, 1H), 4.75-4.39 (m, |
| | | 1H), 4.28-3.99 (m, 1H), 3.59-3.50 (m, |
| | | 2H), 3.18-3.05 (m, 2H), 2.90-2.76 (m, |
| | | 1H), 2.65-2.59 (m, 2H), 2.47-2.40 (m, |
| | | 2H), 2.12-2.03 (m, 2H), 1.87-1.76 (m, |
| | | 2H), 1.72-1.65 (m, 1H), 1.61-1.53 (m, |
| | | 1H), 1.52-1.40 (m, 4H) |
| 65 | [M + H]⁺ = 551.1 | ¹H NMR (DMSO-d₆, 400 MHz) δ 11.66 |
| | | (br, 1H), 9.29 (s, 1H), 8.76-8.75 (m, |
| | | 1H), 8.23-8.17 (m, 2H), 8.14 (d, J = 6 |
| | | Hz, 1H), 8.11-8.08 (m, 1H), 8.03-7.94 |
| | | (m, 2H), 7.34-7.25 (m, 2H), 7.15-7.07 |
| | | (m, 2H), 6.91-6.82 (m, 1H), 6.58-6.53 |
| | | (m, 1H), 6.35 (d, J = 2 Hz, 1H), 6.16- |
| | | 6.08 (m, 1H), 5.71-5.65 (m, 1H), 4.62- |
| | | 4.58 (m, 1H), 4.20-4.18 (m, 1H), 3.48- |
| | | 3.43 (m, 1H), 3.29-3.23 (m, 1H), 2.86- |
| | | 2.80 (m, 1H), 2.12-2.07 (m, 2H), 1.56- |
| | | 1.43 (m, 2H) |
| 104 | [M + H]⁺ = 550.2 | ¹H NMR (CD₃OD, 400 MHz) δ 8.19 (s, |
| | | 1H), 8.12 (t, J = 8.8 Hz, 1H), 8.00 (d, J = |
| | | 6.0 Hz, 1H), 7.42 (d, J = 7.6 Hz, 2H), |
| | | 7.27 (t, J = 8.0 Hz, 2H), 7.13-7.07 (m, |
| | | 1H), 7.07-7.01 (m, 2H), 6.97 (t, J = 7. 2 |
| | | Hz, 1H), 6.87-6.77 (m, 1H), 6.49-6.43 |
| | | (m, 1H), 6.39 (d, J = 2. 0 Hz, 1H), 6.25- |
| | | 6.17 (m, 1H), 5.79-5.72 (m, 1H), 4.73 |
| | | (d, J = 12.4 Hz, 1H), 4.27 (d, J = 13.2 |
| | | Hz, 1H), 3.38 (t, J = 12. 4 Hz, 2H), 3.01- |
| | | 2.90 (m, 1H), 2.31-2.17 (m, 2H), 1.72- |
| | | 1.58 (m, 2H) |
| 107 | [M + H]⁺ = 582.2 | ¹H NMR (CD₃OD, 400 MHz) δ 8.18 (s, |
| | | 1H), 8.13-8.05 (m, 1H), 7.98 (d, J = 6.0 |
| | | Hz, 1H), 7.13-7.00 (m, 3H), 6.83 (dd, J = |
| | | 10.8, 16.8 Hz, 1H), 6.42 (dd, J = 2.0, |
| | | 6.0 Hz, 1H), 6.21 (dd, J = 2.0, 16.8 Hz, |
| | | 1H), 6.01 (d, J = 2.4 Hz, 1H), 5.78-5.72 |
| | | (m, 1H), 4.73 (d, J = 13.2 Hz, 1H), 4.32- |
| | | 4.24 (m, 1H), 4.23-4.15 (m, 2H), 4.02 |
| | | (dd, J = 5.6, 8.8 Hz, 2H), 3.62-3.52 (m, |
| | | 1H), 3.44-3.37 (m, 2H), 3.03-2.91 (m, |
| | | 1H), 2.33-2.17 (m, 2H), 1.73-1.59 (m, |
| | | 2H) |
| 109 | [M + H]⁺ = 528.3 | ¹H NMR (400 MHz, DMSO-d₆) δ 11.62 |
| | | (br, 1H), 8.21 (s, 1H), 7.98-7.83 (m, |
| | | 3H), 7.22-7.16 (m, 1H), 7.06 (s, 1H), |
| | | 7.02 (d, J = 8.8 Hz, 1H), 6.90-6.79 (m, |
| | | 2H), 6.20-6.15 (m, 1H), 6.14-6.08 (m, |
| | | 1H), 5.90 (d, J = 1.6 Hz, 1H), 5.70-5.61 |
| | | (m, 1H), 4.64-4.54 (m, 1H), 4.26-4.14 |
| | | (m, 2H), 3.47-3.42 (m, 2H), 2.82 (m, |
| | | 1H), 2.27-2.19 (m, 2H), 2.08 (m, 2H), |
| | | 1.85-1.77 (m, 2H), 1.69-1.60 (m, 2H), |
| | | 1.53-1.42 (m, 2H) |
| 129 | [M + H]⁺ = 557.2 | ¹H NMR (DMSO-d₆, 400 MHz) δ 11.63 |
| | | (s, 1H), 8.17-8.14 (m, 1H), 8.00 (d, J = |
| | | 6 Hz, 1H), 7.94-7.88 (m, 2H), 7.24- |
| | | 7.18 (m, 1H), 7.10-6.99 (m, 2H), 6.92- |
| | | 6.79 (m, 1H), 6.29-6.23 (m, 1H), 6.15- |
| | | 6.07 (m, 1H), 5.91 (d, J = 2.0 Hz, 1H), |
| | | 5.73-5.62 (m, 1H), 4.59 (m, 1H), 4.24- |
| | | 4.11 (m, 1H), 3.94 (t, J = 8 Hz, 2H), 3.73- |
| | | 3.62 (m, 2H), 3.49-3.41 (m, 2H), 3.18- |
| | | 3.11 (m, 1H), 2.85-2.79 (m, 1H), 2.13- |
| | | 2.04 (m, 8H), 1.56-1.41 (m, 2H) |
| 130 | [M + H]⁺ = 572.6 | ¹H NMR (CD₃OD, 400 MHz) δ 8.19 (s, |
| | | 1H), 8.10 (t, J = 8.8 Hz, 1H), 7.92 (d, J = |
| | | 6.0 Hz, 1H), 7.12-6.98 (m, 3H), 6.88- |
| | | 6.77 (m, 1H), 6.39-6.33 (m, 1H), 6.25- |
| | | 6.16 (m, 1H), 5.95 (d, J = 2.0 Hz, 1H), |
| | | 5.78-5.72 (m, 1H), 4.73 (d, J = 14.0 |
| | | Hz, 1H), 4.27 (d, J = 14.4 Hz, 1H), 3.97 |
| | | (d, J = 8.8 Hz, 2H), 3.74 (d, J = 8.8 Hz, |
| | | 2H), 3.38 (t, J = 12.0 Hz, 2H), 2.96 (t, J = |
| | | 12.8 Hz, 1H), 2.24 (t, J = 11.2 Hz, 2H), |
| | | 2.00-1.88 (m, 1H), 1.73-1.59 (m, 2H), |
| | | 0.97 (d, J = 6.8 Hz, 6H) |
| 131 | [M + H]⁺ = 540.2 | ¹H NMR (CDCl₃, 400 MHz) δ 9.59-9.34 |
| | | (m, 1H), 8.94-8.80 (m, 1H), 8.50 (s, 1H), |
| | | 8.04 (d, J = 6.0 Hz, 1H), 7.19 (d, J = 4.0 |
| | | Hz, 1H), 7.01-6.93 (m, 2H), 6.92 (s, |
| | | 1H), 6.65 (dd, J = 10.8, 16.8 Hz, 1H), |
| | | 6.32 (dd, J = 2.0, 16.8 Hz, 1H), 6.19 (dd, |
| | | J = 2.0, 6.0 Hz, 1H), 5.89 (d, J = 2.0 Hz, |
| | | 1H), 5.73 (m, 1H), 4.92 (m, 1H), 4.33- |
| | | 4.10 (m, 1H), 3.65 (m, 2H), 3.41 (m, |
| | | 2H), 3.37-3.28 (m, 1H), 3.18-3.07 (m, |
| | | 1H), 2.93-2.80 (m, 1H), 2.31-2.20 (m, |
| | | 2H), 1.78-1.66 (m, 2H), 1.63 (m, 2H), |
| | | 0.80-0.70 (m, 1H), 0.29 (q, J = 4.4 Hz, |
| | | 1H) |
| 135 | [M + H]⁺ =551.3 | ¹H NMR (CDCl₃, 400 MHz) δ 9.99 (br, |
| | | 1H), 8.98 (m, 1H), 8.46 (s, 1H), 8.29- |
| | | 8.08 (m, 2H), 8.01-7.78 (m, 1H), 7.73- |
| | | 7.51 (m, 2H), 7.26-7.16 (m, 2H), 7.08- |
| | | 6.97 (m, 2H), 6.93 (b, 1H), 6.90-6.83 |
| | | (m, 1H), 6.70-6.58 (m, 1H), 6.56-6.48 |
| | | (m, 1H), 6.32 (d, J = 17.2 Hz, 1H), 5.73 |
| | | (d, J = 10.4 Hz, 1H), 5.04-4.78 (m, 1H), |
| | | 4.21 (m, 1H), 3.40-3.23 (m, 1H), 3.18- |
| | | 3.01 (m, 1H), 2.96-2.73 (m, 1H), 2.24 |
| | | (m 2H), 1.80-1.68 (m, 2H) |
| 140 | [M + H]⁺ = 596.2 | ¹H NMR (DMSO-d₆, 400 MHz) δ 11.62 |
| | | (br, 1H), 8.16-8.11 (m, 1H), 8.03 (d, J = |
| | | 5.6 Hz, 1H), 7.96-7.86 (m, 2H), 7.19 |
| | | (dd, J = 2.4, 11.2 Hz, 1H), 7.08-7.05 |
| | | (m, 1H), 7.05-7.00 (m, 1H), 6.90-6.81 |
| | | (m, 1H), 6.23 (dd, J = 2.0, 5.6 Hz, 1H), |
| | | 6.14-6.04 (m, 2H), 5.72-5.62 (m, 1H), |
| | | 4.67-4.49 (m, 1H), 4.24-4.09 (m, 1H), |
| | | 3.77-3.60 (m, 1H), 3.52-3.45 (m, 2H), |
| | | 3.44-3.33 (m, 3H), 3.27-3.21 (m, 1H), |
| | | 2.93-2.75 (m, 1H), 2.29-2.18 (m, 1H), |
| | | 2.16-1.99 (m, 3H), 1.55-1.38 (m, 2H) |
| 141 | [M + H]⁺ = 558.2 | ¹H NMR (DMSO-d₆, 400 MHz) δ 11.62 |
| | | (br, 1H), 8.14 (s, 1H), 8.06 (d, J = 5.6 |
| | | Hz, 1H), 7.94-7.85 (m, 2H), 7.23-7.13 |
| | | (m, 1H), 7.07-6.98 (m, 2H), 6.91-6.78 |
| | | (m, 1H), 6.46 (s, 1H), 6.28-6.23 (m, |
| | | 1H), 6.18-6.04 (m, 1H), 5.73-5.59 (m, |
| | | 1H), 4.58 (m, 1H), 4.22-4.07 (m, 2H), |
| | | 3.97 (m, 1H), 3.91 (m, 1H), 3.57-3.38 |
| | | (m, 3H), 3.27-3.19 (m, 1H), 2.86-2.74 |
| | | (m, 2H), 2.47-2.41 (m, 1H), 2.14-2.00 |
| | | (m, 2H), 1.56-1.40 (m, 2H), 1.18-1.11 |
| | | (d, J = 6.4 Hz, 3H) |
| 142 | [M + H]⁺ = 606.1 | ¹H NMR (DMSO-d₆, 400 MHz,) δ 11.67 |
| | | (s, 1H), 8.17 (s, 1H), 8.10 (s, 1H), 8.04 |
| | | (d, J = 5.6 Hz, 1H), 7.87-7.82 (m, 1H), |
| | | 7.19 (d, J = 8.0 Hz, 1H), 7.09 (s, 1H), |
| | | 6.88-6.82 (m, 1H), 6.51 (s, 1H), 6.18- |
| | | 6.07 (m, 2H), 5.69-5.65(m, 1H), 4.60 (d, |
| | | J = 11.6 Hz, 1H), 4.23-4.11 (m, 2H), |
| | | 3.48 (m, 1H), 3.40-3.35 (m, 1H), 2.85 (t, |
| | | J = 11.6 Hz, 1H), 2.50-2.45 (m, 2H), |
| | | 2.05-1.99(m, 4H), 1.53-1.483 (m, 2H), |
| | | 1.23 (s, 6H) |
| 144 | [M + H]⁺ = 598.2 | ¹H NMR (CD₃OD, 400 MHz) δ 8.18 (s, |
| | | 1H), 8.12-8.04 (m, 1H), 7.98 (d, J = 6.0 |
| | | Hz, 1H), 7.12-7.07 (m, 1H), 7.06 (s, |
| | | 1H), 7.05-7.00 (m, 1H), 6.87-6.77 (m, |
| | | 1H), 6.45-6.40 (m, 1H), 6.24-6.18 (m, |
| | | 1H), 6.05 (d, J = 2.0 Hz, 1H), 5.77-5.73 |
| | | (m, 1H), 4.73 (m, 1H), 4.30-4.21 (m, |
| | | 3H), 3.93 (m, 2H), 3.43-3.35 (m, 2H), |
| | | 3.01-2.92 (m, 1H), 2.29-2.19 (m, 2H), |
| | | 1.71-1.59 (m, 2H) |
| 145 | [M + H]⁺ = 564.2 | ¹H NMR (DMSO-d₆, 400 MHz) δ 11.62 |
| | | (br, 1H), 8.20-8.13 (m, 1H), 8.05 (d, J = |
| | | 6.0 Hz, 1H), 7.95-7.87 (m, 2H), 7.20 |
| | | (dd, J = 2.4, 11.2 Hz, 1H),7.01-7.064 |
| | | (m, 2H), 6.85 (dd, J = 10.4, 16.4 Hz, 1H), |
| | | 6.27 (dd, J = 1.6, 6.4 Hz, 1H), 6.16 |
| | | (d, J = 1.6 Hz, 1H), 6.11 (dd, J = |
| | | 3.2, 18.8 Hz, 1H), 5.67 (dd, J = 2.4, 10.4 |
| | | Hz, 1H), 4.58 (m, 1H), 4.17 (m, 1H), |
| | | 3.82 (t, J = 13.6 Hz, 2H), 3.58 (t, J = 7.2 |
| | | Hz, 2H), 3.43 (m, 1H), 3.26-3.23 (m, |
| | | 1H), 2.85-2.76 (m, 1H), 2.57-2.54 (m, |
| | | 2H), 2.08 (m, 2H), 1.52-1.43 (m, 2H) |
| 146 | [M + H]⁺ = 558.3 | ¹H NMR (CD₃OD, 400 MHz) δ 8.18 (s, |
| | | 1H), 8.09 (t, J = 8.8 Hz, 1H), 7.85 (d, J = |
| | | 6.0 Hz, 1H), 7.10-7.04 (m, 2H), 7.02- |
| | | 6.96 (m, 1H), 6.82 (dd, J= 10.6, 16.8 |
| | | Hz, 1H), 6.29 (dd, J = 2.0, 6.0 Hz, 1H), |
| | | 6.21 (dd, J = 2.0, 16.8 Hz, 1H), 6.09 (d, |
| | | J = 2.0 Hz, 1H), 5.75 (dd, J= 2.0, 10.4 |
| | | Hz, 1H), 4.80-4.66 (m, 1H), 4.37-4.18 |
| | | (m, 1H), 3.99-3.92 (m, 2H), 3.91-3.82 |
| | | (m, 1H), 3.60-3.50 (m, 2H), 3.42-3.36 |
| | | (m, 2H), 3.03-2.88 (m, 1H), 2.30-2.16 |
| | | (m, 2H), 2.01-1.90 (m, 2H), 1.74-1.58 |
| | | (m, 2H), 1.56-1.43 (m, 2H) |
| 147 | [M + H]⁺ = 572.3 | ¹H NMR (CD₃OD, 400 MHz) δ 8.18 (s, |
| | | 1H), 8.11-8.04 (m, 1H), 7.98 (d, J = 6.0 |
| | | Hz, 1H), 7.08-7.03 (m, 2H), 7.01 (d, J = |
| | | 9.2 Hz, 1H), 6.82 (dd, J = 10.8, 16.8 Hz, |
| | | 1H), 6.38 (s, 1H), 6.34-6.28 (m, 1H), |
| | | 6.21 (dd, J = 1.6, 16.8 Hz, 1H), 5.75 (dd, |
| | | J = 1.6, 10.8 Hz, 1H), 4.73 (m, 1H), 4.26 |
| | | (m, 1H), 4.04-3.94 (m, 1H), 3.72-3.62 |
| | | (m, 1H), 3.45-3.36 (m, 5H), 3.35 (m, |
| | | 1H), 3.24-3.13 (m, 2H), 3.02-2.91 (m, |
| | | 1H), 2.31.2.19 (m, 2H), 2.07-1.97 (m, |
| | | 1H), 1.87-1.75 (m, 1H), 1.72-1.59 (m, |
| | | 2H), 1.58-1.48 (m, 2H) |
| 148 | [M + H]⁺ = 576.2 | ¹H NMR (CD₃OD, 400 MHz) δ 8.19 (s, |
| | | 1H), 8.13-8.05 (m, 1H), 7.96 (d, J = 6.0 |
| | | Hz, 1H), 7.10-7.04 (m, 2H), 7.04-6.99 |
| | | (m, 1H), 6.82 (dd, J = 10.8, 16.8 Hz, |
| | | 1H), 6.34 (dd, J = 2.0, 6.0 Hz, 1H), 6.21 |
| | | (dd, J = 2.0, 16.8 Hz, 1H), 6.04 (d, J = |
| | | 2.0 Hz, 1H), 5.75 (dd, J = 2.0, 10.8 Hz, |
| | | 1H), 4.73 (m, 1H), 4.32-4.21 (m, 1H), |
| | | 3.80-3.73 (m, 2H), 3.72-3.65 (m, 2H), |
| | | 3.37 (m, 2H), 3.00-2.92 (m, 1H), 2.58- |
| | | 2.47 (m, 2H), 2.29-2.18 (m, 2H), 1.72- |
| | | 1.58 (m, 2H) |
| 173 | [M + H]⁺ = 597.3 | ¹H NMR (DMSO-d₆, 400 MHz) δ 13.34 |
| | | (s, 1H), 8.60 (s, 1H), 8.20 (s, 1H), 8.03 |
| | | (d, J = 5.8 Hz, 1H), 7.62 (t, J = 8.8 Hz, |
| | | 1H), 7.24-7.12 (m, 1H), 7.07-6.95 (m, |
| | | 1H), 6.90-6.74 (m, 1H), 6.26-6.19 (m, |
| | | 1H), 6.15-6.02 (m, 2H), 5.68-5.59 (m, |
| | | 1H), 4.52 (m, 1H), 4.14 (m, 1H), 3.72- |
| | | 3.62 (m, 2H), 3.53-3.42 (m, 2H), 3.42- |
| | | 3.37 (m, 2H), 2.94-2.83 (m, 1H), 2.46- |
| | | 2.43 (m, 1H), 2.28-2.20 (m, 1H), 2.11- |
| | | 1.99 (m, 3H), 1.72-1.59 (m, 2H) |
| 174 | [M + H]⁺ = 559.3 | ¹H NMR (DMSO-d₆, 400 MHz) δ 13.38 |
| | | (s, 1H), 8.64 (s, 1H), 8.25 (d, J = 1.2 Hz, |
| | | 1H), 7.92 (d, J = 5.6 Hz, 1H), 7.66 (m, |
| | | 1H), 7.23 (dd, J = 2.4, 10.8 Hz, 1H), |
| | | 7.05 (dd, J = 2.0, 8.8 Hz, 1H), 6.87 (dd, |
| | | J = 10.4, 16.4 Hz, 1H), 6.63 (d, J = 7.6 |
| | | Hz, 1H), 6.20 (dd, J = 2.4, 6.0 Hz, 1H), |
| | | 6.12 (dd, J = 2.4, 16.6 Hz, 1H), 6.01 (d, |
| | | J = 2.2 Hz, 1H), 5.72-5.65 (m, 1H), |
| | | 4.60-4.44 (m, 1H), 4.17 (m, 1H), 3.97- |
| | | 3.88 (m, 1H), 3.88-3.82 (m, 2H), 3.79- |
| | | 3.66 (m, 1H), 3.42-3.36 (m, 2H), 3.00- |
| | | 2.86 (m, 1H), 2.65-2.59 (m, 1H), 2.13- |
| | | 2.02 (m, 2H), 1.89-1.82 (m, 2H), 1.75- |
| | | 1.61 (m, 2H), 1.45-1.34 (m, 2H) |
| 175 | [M + H]⁺ = 565.3 | ¹H NMR (DMSO-d₆, 400 MHz) δ 13.36 |
| | | (br, 1H), 8.63 (s, 1H), 8.23 (s, 1H), |
| | | 8.07 (d, J = 5.6 Hz, 1H), 7.66 (td, J = |
| | | 8.4, 5.6 Hz, 1H), 7.22 (dd, J = 10.8, 2.4 |
| | | Hz, 1H), 7.05 (dd, J = 8.8, 2.4 Hz, 1H), |
| | | 6.86 (dd, J = 16.4, 10.4 Hz, 1H), 6.29 |
| | | (dd, J = 6.0, 2.0 Hz, 1H), 6.18 (d, J = |
| | | 1.6 Hz, 1H), 6.11 (dd, J = 16.4, 2.4 Hz, |
| | | 1H), 5.67 (dd, J = 10.8, 2.4 Hz, 1H), |
| | | 4.62-4.38 (m, 1H), 4.22-4.06 (m, 1H), |
| | | 3.82 (m, 2H), 3.76-3.66 (m, 1H), |
| | | 3.58 (m, 2H), 2.98-2.84 (m, 1H), 2.61- |
| | | 2.54 (m, 3H), 2.14-2.00 (m, 2H), |
| | | 1.78-1.53 (m, 2H) |
| 176 | [M + H]⁺= 589.1 | ¹H NMR (DMSO-d₆, 400 MHz) δ 13.36 |
| | | (br, 1H) 8.62 (s, 1H) 8.22 (s, 1H) 8.02 |
| | | (d, J = 5.76 Hz, 1H) 7.54-7.73 (m, 1H) |
| | | 7.20 (dd, J = 10.94, 2.56 Hz, 1H) 7.04 |
| | | (dd, J = 8.63, 2.00 Hz, 1H) 6.86 (dd, J = |
| | | 16.64, 10.38 Hz, 1H) 6.20 (dd, J = 5.68, |
| | | 1.94 Hz, 1H) 6.11 (dd, J = 16.76, 2.40 |
| | | Hz, 1H) 6.04 (d, J = 2.00 Hz, 1H) 5.67 |
| | | (dd, J = 10.44, 2.44 Hz, 1H), 4.42-4.57 |
| | | (m, 1H) 4.16 (m, 1H) 4.02 (m, 2H) |
| | | 3.71 (s, 1H) 3.51 (m, 2H) 3.34 (s, 6H) |
| | | 3.30 (s, 3H) 2.92 (m, 1H) 2.06 (m, 2H) |
| | | 1.66 (m, 2H), |
| 177 | [M + H]⁺ = 599.3 | ¹H NMR (DMSO-d₆, 400 MHz) δ 13.37 |
| | | (s, 1H), 8.63 (s, 1H), 8.23 (s, 1H), 8.06 |
| | | (d, J = 5.8 Hz, 1H), 7.71-7.61 (m, 1H), |
| | | 7.34 (s, 1H), 7.27-7.20 (m, 1H), 7.11- |
| | | 7.00 (m, 1H), 6.94-6.80 (m, 1H), 6.38- |
| | | 6.29 (m, 1H), 6.16-6.08 (m, 2H), 5.72- |
| | | 5.61 (m, 1H), 4.56-4.45 (m, 1H), 4.19 |
| | | (d, J = 9.8 Hz, 2H), 4.15 (m, 1H), 3.93 |
| | | (m, 2H), 3.76-3.67 (m, 1H), 2.96-2.86 |
| | | (m, 1H), 2.06 (m, 2H), 1.75-1.61 (m, |
| | | 2H) |
| 178 | [M + H]⁺ = 559.2 | ¹H NMR (DMSO-d₆, 400 MHz) δ 13.37 |
| | | (s, 1H), 8.60-8.67 (m, 1H), 8.21-8.26 |
| | | (m, 1H), 8.03 (d, J = 6.00 Hz, 1H), 7.63- |
| | | 7.71 (m, 1H), 7.21-7.26 (m, 1H), |
| | | 7.04-7.09 (m, 1H), 6.82-6.92 (m, 1H), |
| | | 6.29 (dd, J = 8.00, 3.60 Hz, 1H), |
| | | 6.12 (dd, J = 19.20, 14.4 Hz, 1H), 5.97 |
| | | (d, J = 2.00 Hz, 1H), 5.66-5.71 (m, 1H), |
| | | 4.51 (m, 1H), 4.17 (m, 1H), 3.84 (d, |
| | | J = 8.80 Hz, 2H) 3.73 (d, J = 8.80 Hz, 2H) |
| | | 3.30 (s, 2H) 3.19 (s, 3H) 2.92 (s, 1H) |
| | | 2.07 (d, J = 12.40 Hz, 2H) 1.67 (s, 2H) |
| | | 1.46 (s, 3H), |
| 179 | [M + H]⁺ = 554.2 | ¹H NMR (DMSO-d₆, 400 MHz) δ 13.37 |
| | | (br, 1H), 8.64 (s, 1H), 8.23 (s, 1H), 8.05 |
| | | (d, J = 5.6 Hz, 1H), 7.68-7.60 (m, 1H), |
| | | 7.22 (dd, J = 2.8, 10.8 Hz, 1H), 7.05 (dd, |
| | | J = 2.0, 8.8 Hz, 1H), 6.86 (dd, J = 10.4, |
| | | 16.8 Hz, 1H), 6.26 (dd, J = 2.0, 5.6 Hz, |
| | | 1H), 6.17-6.06 (m, 2H), 5.68 (dd, J = |
| | | 2.4, 10.8 Hz, 1H), 4.51 (m, 1H), 4.17 (m, |
| | | 1H), 3.77-3.67 (m, 2H), 3.66-3.59 (m, |
| | | 1H), 3.58-3.51 (m, 1H), 3.50-3.39 (m, |
| | | 2H), 2.96-2.86 (m, 1H), 2.41-2.30 (m, |
| | | 2H), 2.28-2.21 (m, 1H), 2.10-2.02 (m, |
| | | 2H), 1.74-1.58 (m, 2H) |
| 180 | [M + H]⁺ = 541.2 | ¹H NMR (DMSO-d₆, 400 MHz) δ 13.43 |
| | | (s, 1H), 8.62 (s, 1H), 8.22 (s, 1H), 8.00 |
| | | (d, J = 5.8 Hz, 1H), 7.63 (t, J = 8.8 Hz, |
| | | 1H), 7.27-7.15 (m, 1H), 7.08-6.98 (m, |
| | | 1H), 6.91-6.79 (m, 1H), 6.23-6.18 (m, |
| | | 1H), 6.15-6.07 (m, 1H), 6.02 (d, J = 1.8 |
| | | Hz, 1H), 5.70-5.65 (m, 1H), 4.51 (m, |
| | | 1H), 4.16 (m, 1H), 3.77-3.68 (m, 1H), |
| | | 3.58 (d, J = 10.2 Hz, 2H), 3.39-3.37 |
| | | (m, 2H), 3.28-3.27 (m, 1H), 2.96-2.87 |
| | | (m, 1H), 2.06 (m, 2H), 1.71-1.61 (m, |
| | | 4H), 0.78-0.69 (m, 1H), 0.19-0.12 (m, |
| | | 1H) |
| 181 | [M + H]⁺ = 585.3 | ¹H NMR (DMSO-d₆, 400 MHz) δ 13.36 |
| | | (s, 1H), 8.63 (s, 1H), 8.23 (d, J = 1.6 Hz, |
| | | 1H), 8.01 (d, J = 5.6 Hz, 1H), 7.72-7.62 |
| | | (m, 1H), 7.22 (dd, J = 2.4, 10.8 Hz, 1H), |
| | | 7.05 (dd, J = 2.0, 8.8 Hz, 1H), 6.86 (dd, |
| | | J = 10.4, 16.8 Hz, 1H), 6.27 (dd, J = 2.0, |
| | | 5.6 Hz, 1H), 6.11 (dd, J = 2.4, 16.8 Hz, |
| | | 1H), 5.90 (d, J = 2.0 Hz, 1H), 5.70-5.64 |
| | | (m, 1H), 4.57-4.46 (m, 1H), 4.16 (m, |
| | | 1H), 3.71 (m, 1H), 3.58-3.48 (m, 4H), |
| | | 3.31 (m, 5H), 2.97-2.85 (m, 1H), 2.06 |
| | | (m, 2H), 1.76-1.70 (m, 4H), 1.70-1.54 |
| | | (m, 2H) |
| 200 | [M + H]⁺ = 576.2 | ¹H NMR (CDCl₃, 400 MHz) δ 8.86-8.79 |
| | | (m, 1H), 8.07 (d, J = 6.0 Hz, 1H), 8.01 |
| | | (s, 1H), 7.61 (d, J = 2.8 Hz, 1H), 7.45 (d, |
| | | J = 3.6 Hz, 1H), 7.00 (d, J = 8.4 Hz, 2H), |
| | | 6.71-6.56 (m, 2H), 6.33 (dd, J = 2.0, |
| | | 16.8 Hz, 1H), 6.25 (d, J = 4.8 Hz, 1H), |
| | | 5.90 (d, J = 2.0 Hz, 1H), 5.74 (dd, J = |
| | | 2.0, 10.8 Hz, 1H), 4.99-4.87 (m, 1H), |
| | | 4.28-4.17 (m, 1H), 3.90-3.68 (m, 4H), |
| | | 3.36-3.25 (m, 1H), 3.24-3.15 (m, 1H), |
| | | 2.91-2.78 (m, 1H), 2.47-2.40 (m, 2H), |
| | | 2.16 (m, 2H), 1.89-1.78 (m, 2H) |
| 201 | [M + H]⁺ = 557.3 | ¹H NMR (CD₃OD, 400 MHz) δ 8.03 (d, |
| | | J = 5.6 Hz, 1H), 8.01-7.93 (m, 1H), 7.79 |
| | | (s, 1H), 7.62 (s, 1H), 7.11-6.99 (m, |
| | | 2H), 6.83 (dd, J = 10.8, 16.8 Hz, 1H), |
| | | 6.71 (br, 1H), 6.43 (s, 1H), 6.39 (d, J = |
| | | 5.6 Hz, 1H), 6.21 (d, J = 17.2 Hz, 1H), |
| | | 5.75 (d, J = 10.8 Hz, 1H), 4.73 (m, 1H), |
| | | 4.32-4.20 (m, 1H), 3.56 (m, 5H), 3.43- |
| | | 3.34 (m, 1H), 3.01-2.89 (m, 1H), 2.73- |
| | | 2.65 (m, 4H), 2.44 (s, 3H), 2.13 (m, 2H), |
| | | 1.84-1.66 (m, 2H) |
| 202 | [M + H]⁺ = 572.3 | ¹H NMR (CD₃OD, 400 MHz) δ 8.02- |
| | | 7.93 (m, 2H), 7.79 (s, 1H), 7.62 (d, J = |
| | | 2.8 Hz, 1H), 7.08 (dd, J = 2.4, 11.2 Hz, |
| | | 1H), 7.02 (d, J = 8.8 Hz, 1H), 6.83 (dd, J = |
| | | 10.8, 16.8 Hz, 1H), 6.71 (d, J = 2.8 |
| | | Hz, 1H), 6.38 (s, 1H), 6.31 (dd, J = 1.6, |
| | | 5.6 Hz, 1H), 6.22 (dd, J = 1.6, 16.8 Hz, |
| | | 1H), 5.76 (dd, J = 1.6, 10.8 Hz, 1H), |
| | | 4.79-4.67 (m, 1H), 4.27 (m, 1H), 3.99 |
| | | (dd, J = 3.6, 12.8 Hz, 1H), 3.72-3.61 |
| | | (m, 1H), 3.57-3.48 (m, 1H), 3.44-3.33 |
| | | (m, 5H), 3.24-3.14 (m, 2H), 3.02-2.89 |
| | | (m, 1H), 2.14 (m, 2H), 2.06-1.96 (m, |
| | | 1H), 1.87-1.66 (m, 3H), 1.61-1.47 (m, |
| | | 2H) |
| 203 | [M + H]⁺ = 558.1 | ¹H NMR (CD₃OD, 400 MHz) δ 7.90 (d, |
| | | J = 7.21 Hz, 2H), 7.71 (s, 1H), 7.58 (d, |
| | | J = 2.40 Hz, 1H), 7.22-7.28 (m, 1H), |
| | | 7.11-7.18 (m, 1H), 6.83 (dd, J = 27.60, |
| | | 6.40 Hz, 1H), 6.69 (s, 2H), 6.19-6.25 |
| | | (m, 1H), 6.16-6.18 (m, 1H), 5.74-5.80 |
| | | (m, 1H), 4.69-4.77 (m, 1H), 4.23-4.33 |
| | | (m, 1H), 4.18 (d, J = 9.20 Hz, 2H), |
| | | 3.99-4.11 (m, 2H), 3.56-3.66 (m, 1H), |
| | | 3.34-3.41 (m, 1H), 3.31 (s, 3H), |
| | | 2.87-2.99 (m, 1H), 2.09-2.21 (m, 2H), |
| | | 1.67-1.84 (m, 2H), 1.57 (s, 3H) |
| 204 | [M + H]⁺ = 553.2 | ¹H NMR (CDCl₃, 400 MHz) δ 8.89-8.78 |
| | | (m, 1H), 8.08 (d, J = 6.0 Hz, 1H), 8.01 |
| | | (s, 1H), 7.61 (d, J = 2.8 Hz, 1H), 7.46 |
| | | (br, 1H), 7.00 (d, J = 6.0 Hz, 2H), 6.70- |
| | | 6.58 (m, 2H), 6.37-6.26 (m, 2H), 5.95 |
| | | (s, 1H), 5.74 (d, J = 10.8 Hz, 1H), 4.99- |
| | | 4.88 (m, 1H), 4.29-4.16 (m, 1H), 3.90- |
| | | 3.75 (m, 2H), 3.70-3.62 (m, 1H), 3.57- |
| | | 3.48 (m, 1H), 3.36-3.15 (m, 3H), 2.89- |
| | | 2.79 (m, 1H), 2.49-2.34 (m, 2H), 2.17 |
| | | (m, 2H), 1.88-1.78 (m, 2H) |

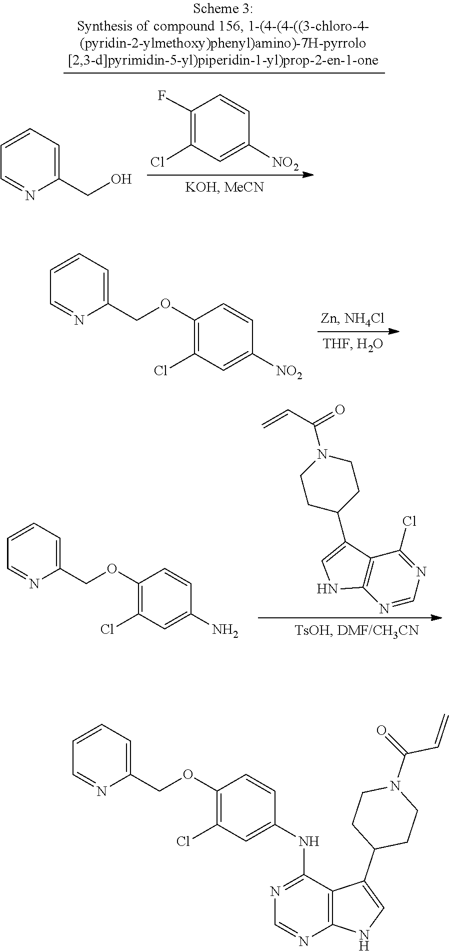

Compounds 29, 30, 35, 36, 37, 39, 52, 68, 73, 75, 77, 80, 81, 85, 86, 87, 88, 89, 94, 98, 99, 102, 103, 106, 108,111, 112, 113, 114, 116, 117, 118, 119, 120, 121, 126, 127, 153, 166, and 198 can be prepared via a similar procedure to the one shown in Scheme 3.

TABLE 4
Characterization of compounds

| Com- | | |
|---|---|---|
| pound | | |
| No. | LC-MS | ¹H NMR |
| 29 | [M + H]⁺ = 503.1 | ¹H NMR (400 MHZ, |
| | | CDCl₃) δ 9.90 (br, 1H), 8.45 (d, J = 4.0 |
| | | Hz, 1H), 8.40 (s, 1H), 7.71 (d, J = 2.4 Hz, |
| | | 1H), 7.54 (d, J = 7.6 Hz, 1H), 7.37 (dd, J = |
| | | 2.8, 8.8 Hz, 1H), 7.24-7.17 (m, 2H), |
| | | 6.89 (s, 1H), 6.68 (s, 1H), 6.63 (dd, J = |
| | | 10.8, 16.8 Hz, 1H), 6.32 (dd, J = 2.0, 16.8 |
| | | Hz, 1H), 5.73 (dd, J = 2.0, 10.4 Hz, 1H), |
| | | 5.33 (s, 2H), 4.94-4.79 (m, 1H), 4.27- |
| | | 4.08 (m, 1H), 3.34-3.14 (m, 1H), 3.04- |
| | | 2.94 (m, 1H), 2.87-2.74 (m, 1H), 2.51 (s, |
| | | 3H), 2.18 (m, 2H), 1.71-1.63 (m, 2H), |
| 30 | [M + H]⁺ = 503.2 | ¹H NMR (CD₃OD, 400 |
| | | MHz) δ 8.49 (d, J = 5.2 Hz, 1H), 8.13 (s, |
| | | 1H), 7.85-7.75 (m, 1H), 7.63 (d, J = 2.8 |
| | | Hz, 1H), 7.51 (d, J = 8.0 Hz, 1H), 7.39 |
| | | (dd, J = 2.8, 8.8 Hz, 1H), 7.30 (dd, J = |
| | | 5.6, 7.2 Hz, 1H), 7.09 (d, J = 8.8 Hz, |
| | | 1H), 7.01 (s, 1H), 6.80 (dd, J = 10.8, |
| | | 16.8 Hz, 1H), 6.20 (dd, J = 2.0, 16.8 Hz, |
| | | 1H), 5.74 (dd, J = 2.0, 10.8 Hz, 1H), |
| | | 4.77-4.63 (m, 2H), 4.59 (m, 10.0 Hz, |
| | | 1H), 4.47-4.36 (m, 2H), 4.22 (m, 1H), |
| | | 3.37 (m, 2H), 3.01-2.83 (m, 1H), 2.25- |
| | | 2.01 (m, 2H), 1.71-1.44 (m, 2H), |
| 35 | [M + H]⁺ = 523.1 | ¹H NMR (CD₃OD, 400 |
| | | MHZ) δ 8.52 (dd, J = 1.2, 4.8 Hz, 1H), |
| | | 8.15 (s, 1H), 7.96 (dd, J = 1.0, 8.0 Hz, |
| | | 1H), 7.69 (d, J = 2.8 Hz, 1H), 7.50-7.36 |
| | | (m, 2H), 7.22 (d, J = 8.8 Hz, 1H), 7.01 |
| | | (s, 1H), 6.81 (dd, J = 10.8, 16.8 Hz, 1H), |
| | | 6.20 (dd, J = 2.0, 16.8 Hz, 1H), 5.74 (dd, |
| | | J = 2.0, 10.8 Hz, 1H), 5.37 (s, 2H), 4.69 |
| | | (m, 1H), 4.22 (m, 1H), 3.35 (m, 2H), 2.98- |
| | | 2.85 (m, 1H), 2.30-2.10 (m, 2H), 1.67- |
| | | 1.51 (m, 2H) |
| 36 | [M + H]⁺ = 507.1 | ¹H NMR (CD₃OD, 400 |
| | | MHz) δ 8.15 (s, 1H), 8.07-7.94 (m, 1H), |
| | | 7.74 (d, J = 2.8 Hz, 1H), 7.59 (dd, J = |
| | | 2.0, 7.6 Hz, 1H), 7.41 (dd, J = 2.8, 8.8 |
| | | Hz, 1H), 7.14 (d, J = 8.8 Hz, 1H), 7.06- |
| | | 6.96 (m, 2H), 6.81 (dd, J = 10.8, 16.8 |
| | | Hz, 1H), 6.20 (dd, J = 2.0, 16.8 Hz, 1H), |
| | | 5.74 (dd, J = 2.0, 10.8 Hz, 1H), 5.21 (s, |
| | | 2H), 4.69 (m, 1H), 4.22 (m, 1H), 3.42- |
| | | 3.33 (m, 2H), 3.00-2.87 (m, 1H), 2.27- |
| | | 2.10 (m, 2H), 1.68-1.51 (m, 2H) |
| 37 | [M + H]⁺ = 507.1 | ¹H NMR (CD₃OD400 |
| | | MHz) δ 8.46-8.40 (m, 1H), 8.15 (s, 1H), |
| | | 7.73-7.67 (m, 2H), 7.56-7.48 (m, 1H), |
| | | 7.42 (dd, J = 2.8, 8.8 Hz, 1H), 7.25 (d, J = |
| | | 8.8 Hz, 1H), 7.01 (s, 1H), 6.80 (dd, J = |
| | | 10.8, 16.8 Hz, 1H), 6.20 (dd, J = 2.0, |
| | | 16.8 Hz, 1H), 5.74 (dd, J = 2.0, 10.8 Hz, |
| | | 1H), 5.32 (d, J = 2.0 Hz, 2H), 4.69 (m, |
| | | 1H), 4.22 (m, 1H), 3.36 (m, 2H), 2.99- |
| | | 2.87 (m, 1H), 2.27-2.11 (m, 2H), 1.67- |
| | | 1.48 (m, 2H) |
| 39 | [M + H]⁺ = 533.1 | ¹H NMR (DMSO-d₆, 400 |
| | | MHz) δ 11.60 (s, 1H), 8.59 (d, J = 4.4 |
| | | Hz, 1H), 8.19-8.16 (m, 1H), 8.04-7.93 |
| | | (m, 2H), 7.88 (td, J = 1.6, 7.6 Hz, 1H), |
| | | 7.62-7.56 (m, 2 H), 7.36 (dd, J = 5.2, |
| | | 7.2 Hz, 1H), 7.19 (d, J = 8.8 Hz, 1H), |
| | | 7.04 (s, 1H), 6.83 (dd, J = 10.4, 16.8 Hz, |
| | | 1H), 6.10 (dd, J = 2.4, 16.8, Hz, 1H), |
| | | 5.66 (dd, J = 2.4, 10.4 Hz, 1H), 5.28 (s, |
| | | 2 H), 4.56 (m, 1H), 4.20-4.10 (m, 1H), |
| | | 3.51 (m, 1H), 3.30-3.21 (m, 1H), 2.83 |
| | | (m, 1H), 2.03 (m, 2H), 1.49-1.37 (m, |
| | | 2H) |
| 52 | [M + H]⁺ = 496.2 | ¹H NMR (DMSO-d₆, 400 |
| | | MHz) δ 11.62 (s, 1H), 9.67 (s, 1H), 8.20 |
| | | (s, 1H), 8.02 (s, 1H), 7.87 (d, J = 2.6 Hz, |
| | | 1H), 7.57 (d, J = 2.6 Hz, 1H), 7.33 (d, J = |
| | | 8.8 Hz, 1H), 7.05 (s, 1H), 6.81 (d, J = |
| | | 10.4 Hz, 1H), 6.15-6.04 (m, 1H), 5.73 |
| | | (s, 2H), 5.69-5.61 (m, 1H), 4.62-4.49 |
| | | (m, 1H), 4.14 (d, J = 13.4Hz, 1H), 3.52 |
| | | (t, J = 11.6 Hz, 1H), 3.28-3.24 (m, 1H), |
| | | 2.84 (t, J = 12.2 Hz, 1H), 2.08-1.99 (m, |
| | | 2H), 1.44 (d, J = 11.7 Hz, 2H) |
| 60 | [M + H]⁺ = 532.3 | ¹H NMR (CD₃OD, 400 |
| | | MHz) δ 8.54 (s, 1 H) 8.31 (s, 1 H) 7.52- |
| | | 7.57 (m, 2 H) 7.30 (s, 1 H) 7.26 (dd, J = |
| | | 8.68, 2.40 Hz, 1 H) 6.97 (d, J = 8.68 Hz, |
| | | 1 H) 6.82 (dd, J = 16.64, 10.58 Hz, 1 H) |
| | | 6.22 (dd, J = 16.80, 1.80 Hz, 1 H) 5.76 |
| | | (dd, J = 10.64, 1.80 Hz, 1 H) 5.43 (s, 2 |
| | | H) 4.70 (m, 1 H) 4.19-4.31 (m, 1 H) |
| | | 4.11 (t, J = 6.00 Hz, 2 H) 3.37-3.48 (m, |
| | | 2 H) 2.96 (m, 1 H) 2.78 (m, 2 H) 2.22 (m, |
| | | 2 H) 2.01-2.08 (m, 2 H) 1.86-1.93 (m, |
| | | 2 H) 1.59-1.71 (m, 2 H) |
| 68 | [M + H]⁺ = 532.3 | ¹H NMR (DMSO-d₆, 400 |
| | | MHz) δ 11.60 (s, 1H), 8.18 (s, 1H), 7.98 |
| | | (s, 1H), 7.81-7.76 (m, 1H), 7.59-7.52 |
| | | (m, 1H), 7.32 (d, J = 8.8 Hz, 1H), 7.04 |
| | | (s, 1H), 6.96 (s, 1H), 6.88-6.78 (m, 1H), |
| | | 6.14-6.06 (m, 1H), 5.69-5.63 (m, 1H), |
| | | 5.15 (s, 2H), 4.56 (d, J = 12.8 Hz, 1H), |
| | | 4.14 (d, J = 12.4 Hz, 1H), 3.99 (t, J = 5.6 |
| | | Hz, 2H), 3.53-3.49 (m, 1H), 3.27-3.23 |
| | | (m, 1H), 2.84-2.82 (m, 1H), 2.73 (t, J = |
| | | 6.4 Hz, 2H), 2.05-2.01 (m, 2H), 1.9 5- |
| | | 1.92 (m, 2H), 1.86-1.78 (m, 2H), 1.51- |
| | | 1.37 (m, 2H) |
| 71 | [M + H]⁺ = 519.3 | ¹H NMR (DMSO-d₆, 400 |
| | | MHz) δ 11.68 (s, 1 H), 8.69-8.57 (m, 2 |
| | | H), 8.18 (s, 1 H), 8.13 (s, 1 H), 7.83 (s, 1 |
| | | H), 7.58 (d, J = 8.88 Hz, 1 H), 7.36 (d, J = |
| | | 6.0 Hz, 1 H), 7.29 (d, J = 8.8 Hz, 1 H), |
| | | 7.07 (s, 1 H), 6.83 (dd, J = 16.8, 10.8 |
| | | Hz, 1 H), 6.10 (br d, J = 16.8 Hz, 1 H), |
| | | 5.66 (dd, J = 10.4, 2.4 Hz, 1 H), 5.21 (s, |
| | | 2 H), 4.55 (d, J = 11.6 Hz, 1 H), 4.23- |
| | | 4.10 (m, 1 H), 4.01 (s, 3 H), 3.57-3.53 |
| | | (m, 1 H), 3.21-3.15 (m, 1 H), 2.90- |
| | | 2.78 (m, 1 H), 2.11-1.96 (m, 2 H), 1.54- |
| | | 1.35 (m, 2 H) |
| 73 | [M + H]⁺ = 528.3 | ¹H NMR (DMSO-d₆, 400 |
| | | MHz) δ 11.81-11.46 (m, 2H), 8.17 (s, |
| | | 1H), 8.04-7.93 (m, 2H), 7.81 (d, J = 2.4 |
| | | Hz, 1H), 7.57-7.45 (m, 2H), 7.31-7.19 |
| | | (m, 2H), 7.03 (d, J = 1.8 Hz, 1H), 6.91- |
| | | 6.74 (m, 1H), 6.54-6.39 (m, 1H), 6.15- |
| | | 6.03 (m, 1H), 5.74-5.59 (m, 1H), 5.31 |
| | | (s, 2H), 4.55 (m, 1H), 4.26-4.02 (m, |
| | | 1H), 3.55-3.44 (m, 1H), 3.28-3.20 (m, |
| | | 1H), 2.96-2.71 (m, 1H), 2.09-1.94 (m, |
| | | 2H), 1.43 (m, 2H) |
| 75 | [M + H]⁺ = 545.2 | ¹H NMR (DMSO-d₆, 400 |
| | | MHz) δ 11.60 (s, 1 H), 8.56 (d, J = 8.4 |
| | | Hz, 1 H), 8.25-8.12 (m, 2 H), 7.98 (s, 1 |
| | | H), 7.85 (dd, J = 5.6, 2.4 Hz, 1 H), 7.62- |
| | | 7.50 (m, 3 H), 7.24 (d, J = 9.2 Hz, 1 H), |
| | | 7.04 (s, 1 H), 6.82 (dd, J = 16.4, 10.4 |
| | | Hz, 1 H), 6.10 (dd, J = 16.4, 2.0 Hz, 1 |
| | | H), 5.66 (dd, J = 10.4, 2.4 Hz, 1 H), 5.41 |
| | | (s, 2 H), 4.55 (m, 1 H), 4.24-4.04 (m, 1 |
| | | H), 3.51 (m, 1 H), 3.27-3.20 (m, 1 H), |
| | | 2.91-2.76 (m, 1 H), 2.16- 1.92 (m, 2 H), |
| | | 1.53-1.32 (m, 2 H) |
| 77 | [M + H]⁺ = 490.2 | ¹H NMR (CD₃OD, 400 |
| | | MHz) δ 9.15 (s, 1H), 8.96 (s, 2H), 8.16 |
| | | (s, 1H), 7.74 (d, J = 2.4 Hz, 1H), 7.48 |
| | | (dd, J = 2.4, 8.8 Hz, 1H), 7.24 (d, J = 9.2 |
| | | Hz, 1H), 7.03 (s, 1H), 6.81 (dd, J = 10.4, |
| | | 16.4 Hz, 1H), 6.20 (dd, J = 2.0, 16.8 Hz, |
| | | 1H), 5.74 (dd, J = 2.0, 10.4 Hz, 1H), |
| | | 5.30 (s, 2H), 4.74-4.65 (m, 1H), 4.28- |
| | | 4.18 (m, 1H), 3.43-3.35 (m, 2H), 3.02- |
| | | 2.89 (m, 1H), 2.26-2.12 (m, 2H), 1.70- |
| | | 1.51 (m, 2H) |
| 80 | [M + H]⁺ = 519.2 | ¹H NMR (DMSO-d₆, 400 |
| | | MHz) δ 11.60 (s, 1 H), 8.36-8.24 (m, 2 |
| | | H), 8.18 (s, 1 H), 7.98 (s, 1 H), 7.89- |
| | | 7.76 (m, 1 H), 7.62-7.52 (m, 1 H), 7.48 |
| | | (s, 1 H), 7.26 (d, J = 8.8 Hz, 1 H), 7.04 |
| | | (s, 1 H), 6.83 (dd, J = 16.8, 10.4 Hz, 1 |
| | | H), 6.10 (dd, J = 16.8, 2.4 Hz, 1 H), 5.77- |
| | | 5.58 (m, 1 H), 5.26 (s, 2 H), 4.55 (d, J = |
| | | 12.8 Hz, 1 H), 4.14 (d, J = 12.8 Hz, 1 |
| | | H), 3.85 (s, 3 H), 3.56-3.48 (m, 1 H), |
| | | 3.28-3.23 (m, 1 H), 2.83 (t, J = 12.4 Hz, |
| | | 1 H), 2.03 (d, J = 12.4 Hz, 2 H), 1.43 (q, |
| | | J = 12.8 Hz, 2 H) |
| 81 | [M + H]⁺ = 506.3 | ¹H NMR (DMSO-d₆, 400 |
| | | MHz) δ 11.60 (s, 1 H), 8.18 (s, 1 H), |
| | | 7.99 (s, 1 H), 7.85-7.76 (m, 1 H), 7.61- |
| | | 7.53 (m, 1 H), 7.43 (d, J = 1.6 Hz, 1 H), |
| | | 7.33 (d, J = 9.2 Hz, 1 H), 7.05 (s, 1 H), |
| | | 6.83 (dd, J = 16.8, 10.4 Hz, 1 H), 6.41 |
| | | (d, J = 1.6 Hz, 1 H), 6.10 (dd, J = 16.8, |
| | | 2.8 Hz, 1 H), 5.74-5.61 (m, 1 H), 5.27 |
| | | (s, 2 H), 4.65-4.54 (m, 1 H), 4.28-4.07 |
| | | (m, 3 H), 3.62-3.44 (m, 1 H), 3.28- |
| | | 3.21 (m, 1 H), 2.83 (br t, J = 11.6 Hz, 1 |
| | | H), 2.14-1.98 (m, 2 H), 1.51-1.31 (m, |
| | | 5 H) |
| 85 | [M + H]⁺ = 520.2 | ¹H NMR (CD₃OD, 400 |
| | | MHz) δ 8.14 (s, 1H), 7.73 (s, 1H), 7.67 |
| | | (d, J = 2.8 Hz, 1H), 7.40 (dd, J = 2.8, 8.8 |
| | | Hz, 1H), 7.32 (s, 1H), 7.20 (d, J = 8.8 |
| | | Hz, 1H), 7.01 (s, 1H), 6.81 (dd, J = 10.4, |
| | | 16.8 Hz, 1H), 6.20 (dd, J = 2.0, 16.8 Hz, |
| | | 1H), 5.74 (dd, J = 2.0, 10.8 Hz, 1H), |
| | | 5.05 (s, 2H), 4.69 (d, J = 14.4 Hz, 1H), |
| | | 4.45 (td, J = 6.4, 13.2 Hz, 1H), 4.28- |
| | | 4.16 (m, 1H), 3.35 (d, J = 3.6 Hz, 2H), |
| | | 3.00-2.86 (m, 1H), 2.25-2.11 (m, 2H), |
| | | 1.66-1.53 (m, 2H), 1.50 (d, J = 6.8 Hz, |
| | | 6H) |
| 86 | [M + H]⁺ = 495.2 | ¹H NMR (DMSO-d₆, 400 |
| | | MHz) δ 11.62 (s, 1H), 9.14 (s, 1H), 8.18 |
| | | (s, 1H), 8.02 (s, 1H), 7.91 (s, 1H), 7.82 |
| | | (d, J = 2.4 Hz, 1H), 7.56 (dd, J1 = 2.4 |
| | | Hz, J2 = 8.8 Hz, 1H) 7.30 (d, J = 9.2 Hz, |
| | | 1H), 7.05 (s, 1H), 6.83 (dd, J1 = 10.8 |
| | | Hz, J2 = 16.8 Hz, 1H), 6.10 (dd, J1 = 2.4 |
| | | Hz, J2 = 16.4 Hz, 1H), 5.66 (dd, J1 = 2.4 |
| | | Hz, J2 = 10.4 Hz, 1H), 5.49 (s, 2H), 4.55 |
| | | (d, J = 13.6 Hz, 1H), 4.14 (d, J = 13.2 |
| | | Hz, 1H), 3.52 (t, J = 1.2 Hz, 1H), 3.30- |
| | | 3.26 (m, 1H), 2.83 (t, J = 1.2 Hz, 1H), |
| | | 2.05-2.01 (m, 2H), 1.44-1.42 (m, 2H) |
| 87 | [M + H]⁺ = 563.1 | ¹H NMR (CD₃OD, 400 |
| | | MHz) δ 8.16 (s, 1H), 8.07 (s, 1H), 7.74 |
| | | (d, J = 2.8 Hz, 1H), 7.47 (dd, J = 2.8, 8.8 |
| | | Hz, 1H), 7.23 (d, J = 8.8 Hz, 1H), 7.03 |
| | | (s, 1H), 6.80 (dd, J = 10.8, 16.8 Hz, 1H), |
| | | 6.20 (dd, J = 2.0, 16.8 Hz, 1H), 5.74 (dd, |
| | | J = 2.0, 10.8 Hz, 1H), 5.52 (s, 2H), 4.72- |
| | | 4.64 (m, 1H), 4.22 (br d, J = 13.2 Hz, |
| | | 1H), 3.45-3.34 (m, 2H), 3.00-2.89 (m, |
| | | 1H), 2.24-2.14 (m, 2H), 1.68-1.52 (m, |
| | | 2H) |
| 88 | [M + H]⁺ = 529.2 | ¹H NMR (CD₃OD, 400 MHZ) δ 8.14 (s, |
| | | 1H), 7.78-7.63 (m, 2H), 7.46-7.34 (m, |
| | | 2H), 7.17-7.07 (m, 2H), 7.11 (s, 1H), |
| | | 6.86-6.75 (m, 1H), 6.25-6.16 (m, 1H), |
| | | 5.79-5.68 (m, 1H), 5.19 (s, 2H), 4.76- |
| | | 4.62 (m, 1H), 4.30-4.18 (m, 1H), 3.43- |
| | | 3.35 (m, 2H), 3.00-2.88 (m, 1H), 2.24- |
| | | 2.15 (m, 2H), 2.12-2.05 (m, 1H), 1.66- |
| | | 1.50 (m, 2H), 1.08-0.91 (m, 4H) |
| 89 | [M + H]⁺ = 529.1 | ¹H NMR (CD₃OD, 400 |
| | | MHz) δ 8.34 (d, J = 5.2 Hz, 1H), 8.15 (s, |
| | | 1H), 7.74 (d, J = 2.4 Hz, 1H), 7.45-7.35 |
| | | (m, 2H), 7.13 (d, J = 8.8 Hz, 1H), 7.10- |
| | | 7.06 (m, 1H), 7.02 (s, 1H), 6.85-6.76 |
| | | (m, 1H), 6.24-6.16 (m, 1H), 5.77-5.70 |
| | | (m, 1H), 5.22 (s, 2H), 4.73-4.65 (m, |
| | | 1H), 4.22 (m, 1H), 3.43-3.34 (m, 2H), |
| | | 2.98-2.89 (m, 1H), 2.18 (m, 2H), 2.02- |
| | | 1.95 (m, 1H), 1.66-1.54 (m, 2H), 1.20- |
| | | 1.13 (m, 2H), 0.90-0.81 (m, 2H) |
| 94 | [M + H]⁺ = 492.3 | ¹H NMR (DMSO-d₆, 400 |
| | | MHz) δ 11.76-11.36 (m, 1H), 8.20- |
| | | 8.17 (m, 1H), 8.17-8.12 (m, 1H), 7.97 |
| | | (s, 1H), 7.84-7.77 (m, 1H), 7.59-7.51 |
| | | (m, 1H), 7.35 (d, J = 8.8 Hz, 1H), 7.20 |
| | | (d, J = 0.8 Hz, 1H), 7.04 (d, J = 2.0 Hz, |
| | | 1H), 6.89-6.86 (m, 1H), 6.86-6.78 (m, |
| | | 1H), 6.10 (dd, J = 2.4, 16.8 Hz, 1H), |
| | | 5.66 (dd, J = 2.4, 10.4 Hz, 1H), 5.23 (s, |
| | | 2H), 4.64-4.47 (m, 1H), 4.21-4.07 (m, |
| | | 1H), 3.74 (s, 3H), 3.56-3.46 (m, 2H), |
| | | 2.91-2.77 (m, 1H), 2.13-1.95 (m, 2H), |
| | | 1.55-1.31 (m, 2H) |
| 95 | [M + H]⁺ = 479.1 | ¹H NMR (CD₃OD, 400 |
| | | MHz) δ 8.16 (s, 1H), 7.99 (s, 1H), 7.72 |
| | | (d, J = 2.8 Hz, 1H), 7.43 (dd, J = 2.8, 8.8 |
| | | Hz, 1H), 7.25-7.18 (m, 2H), 7.02 (s, |
| | | 1H), 6.81 (dd, J = 10.8, 16.8 Hz, 1H), |
| | | 6.20 (dd, J = 2.0, 16.8 Hz, 1H), 5.74 (dd, |
| | | J = 2.0, 10.8 Hz, 1H), 5.27 (s, 2H), 4.75- |
| | | 4.64 (m, 1H), 4.22 (d, J = 12.8 Hz, 1H), |
| | | 3.41-3.32 (m, 2H), 2.99-2.90 (m, 1H), |
| | | 2.25-2.11 (m, 2H), 1.70-1.48 (m, 2H) |
| 98 | [M + H]⁺ = 519.2 | ¹H NMR (CD₃OD, 400 |
| | | MHz) δ 8.24 (d, J = 2.8 Hz, 1H), 8.15 (s, |
| | | 1H), 7.71 (d, J = 2.4 Hz, 1H), 7.62 (d, J = |
| | | 8.8 Hz, 1H), 7.47 (dd, J = 2.8, 8.8 Hz, |
| | | 1H), 7.40 (dd, J = 2.4, 8.8 Hz, 1H), 7.14 |
| | | (d, J = 8.8 Hz, 1H), 7.01 (s, 1H), 6.80 |
| | | (dd, J = 10.8, 16.8 Hz, 1H), 6.20 (dd, J = |
| | | 2.0, 16.8 Hz, 1H), 5.74 (dd, J = 2.0, 10.8 |
| | | Hz, 1H), 5.20 (s, 2H), 4.73-4.64 (m, |
| | | 1H), 4.22 (m, 1H), 3.90 (s, 3H), 3.42- |
| | | 3.33 (m, 2H), 2.97-2.87 (m, 1H), 2.24- |
| | | 2.10 (m, 2H), 1.66-1.51 (m, 2H) |
| 99 | [M + H]⁺ = 519.2 | ¹H NMR (CD₃OD, 400 |
| | | MHz) δ 8.24 (d, J = 2.8 Hz, 1H), 8.15 (s, |
| | | 1H), 7.71 (d, J = 2.4 Hz, 1H), 7.62 (d, J = |
| | | 8.8 Hz, 1H), 7.47 (dd, J = 2.8, 8.8 Hz, |
| | | 1H), 7.40 (dd, J = 2.4, 8.8 Hz, 1H), 7.14 |
| | | (d, J = 8.8 Hz, 1H), 7.01 (s, 1H), 6.80 |
| | | (dd, J = 10.8, 16.8 Hz, 1H), 6.20 (dd, J = |
| | | 2.0, 16.8 Hz, 1H), 5.74 (dd, J = 2.0, 10.8 |
| | | Hz, 1H), 5.20 (s, 2H), 4.73-4.64 (m, |
| | | 1H), 4.22 (m, 1H), 3.90 (s, 3H), 3.42- |
| | | 3.33 (m, 2H), 2.97-2.87 (m, 1H), 2.24- |
| | | 2.10 (m, 2H), 1.66-1.51 (m, 2H) |
| 102 | [M + H]⁺ = 528.4 | ¹H NMR (DMSO-d₆, 400 |
| | | MHz) δ 11.62 (s, 1H), 8.58-8.56 (m, |
| | | 1H), 8.18 (s, 1H), 8.03-7.98 (m, 2H), |
| | | 7.82 (s, 1H), 7.56-7.54 (m, 2H), 7.36- |
| | | 7.34 (m, 1H), 7.27-7.25 (m, 1H), 7.05 |
| | | (s, 1H), 6.95-6.77 (m, 2H), 6.11 (d, J = |
| | | 16.4 Hz, 1H), 5.67 (d, J = 10.4 Hz, 1H), |
| | | 5.31 (s, 2H), 4.57-7.55 (m, 1H), 4.15- |
| | | 4.13 (m, 1H), 3.57-3.47 (m, 1H), 3.29- |
| | | 3.21 (m, 1H), 2.91-2.76 (m, 1H), 2.05- |
| | | 2.01 (m, 2H), 1.47-1.41 (m, 2H) |
| 103 | [M + H]⁺ = 563.2 | ¹H NMR (DMSO-d₆, 400 |
| | | MHz) δ 11.63 (br, 1H), 8.58 (s, 1H), 8.20 |
| | | (s, 1H), 8.06-8.00 (m, 1H), 8.03 (s, 1H), |
| | | 7.89 (d, J = 2.6 Hz, 1H), 7.58 (d, J = 2.6 |
| | | Hz, 1H), 7.30 (d, J = 9.0 Hz, 1H), 7.06 |
| | | (s, 1H), 6.88-6.76 (m, 1H), 6.15-6.04 |
| | | (m, 1H), 5.71-5.63 (m, 1H), 5.60 (s, |
| | | 2H), 4.62-4.50 (m, 1H), 4.13 (s, 1H), |
| | | 3.58-3.48 (m, 1H), 3.28-3.24 (m, 1H), |
| | | 2.90-2.77 (m, 1H), 2.03 (m, 2H), 1.51- |
| | | 1.38 (m, 2H) |
| 106 | [M + H]⁺ = 507.2 | ¹H NMR (400 MHZ, |
| | | DMSO-d₆) δ 11.57 (br, 1H), 8.61 (d, J = |
| | | 4.8 Hz, 1H), 8.07 (s, 1H), 7.96 (s, 1H), |
| | | 7.89 (t, J = 7.6 Hz, 1H), 7.61-7.57 (m, |
| | | 1H), 7.56-7.49 (m, 1 H), 7.41-7.35 (m, |
| | | 1H), 7.12 (d, J = 9.2 Hz, 1H), 7.04 (s, |
| | | 1H), 6.84 (dd, J = 10.4, 16.4 Hz, 1H), |
| | | 6.10 (dd, J = 1.6, 16.8 Hz, 1H), 5.72- |
| | | 5.61 (m, 1H), 5.36 (s, 2 H), 4.56 (m, 1 |
| | | H), 4.22-4.10 (m, 1H), 3.42 (m, 1H), |
| | | 3.29-3.23 (m, 1H), 2.88-2.77 (m, 1H), |
| | | 2.11-2.01 (m, 2H), 1.52-1.40 (m, 2H) |
| 108 | [M + H]⁺ = 520.3 | ¹H NMR (400 MHZ, |
| | | DMSO-d₆) δ 10.35-9.65 (m, 1H), 8.39 |
| | | (s, 1H), 8.23 (s, 1H), 7.87 (s, 1H), 7.56 |
| | | (s, 1H), 7.49 (d, J = 2.0 Hz, 1H), 7.23 (d, |
| | | J = 8.4 Hz, 1H), 7.12 (s, 1H), 6.96 (d, J = |
| | | 8.6 Hz, 1H), 6.88-6.78 (m, 1H), 6.15- |
| | | 6.02 (m, 1H), 5.72-5.60 (m, 1H), 5.36 |
| | | (s, 2H), 4.58-4.47 (m, 1H), 4.46-4.36 |
| | | (m, 1H), 4.18-4.05 (m, 1H), 3.46-3.44 |
| | | (m, 1H), 3.22-3.19 (m, 1H), 2.81 (s, |
| | | 1H), 2.04-1.94 (m, 2H), 1.50-1.40 (m, |
| | | 2H), 1.34 (d, J = 6.8 Hz, 6H) |
| 110 | M + H]⁺ = 503.1 | ¹H NMR (DMSO-d₆, 400 |
| | | MHz) δ = 11.61 (s, 1H), 8.44 (d, J = 4.8 |
| | | Hz, 1H), 8.23-8.11 (m, 1H), 7.99 (s, |
| | | 1H), 7.88-7.80 (m, 1H), 7.58-7.50 (m, |
| | | 1H), 7.40 (s, 1H), 7.21 (d, J = 8.8 Hz, |
| | | 2H), 7.04 (s, 1H), 6.83 (dd, J = 10.4, |
| | | 16.8 Hz, 1H), 6.10 (dd, J = 2.4, 16.8 Hz, |
| | | 1H), 5.66 (dd, J = 2.4, 10.4 Hz, 1H), |
| | | 5.23 (s, 2H), 4.63-4.47 (m, 1H), 4.14 |
| | | (m, 1H), 3.55-3.47 (m, 1H), 3.27-3.22 |
| | | (m, 1H), 2.87-2.79 (m, 1H), 2.35 (s, |
| | | 3H), 2.07-1.98 (m, 2H), 1.49-1.36 (m, |
| | | 2H) |
| 112 | [M + H]⁺ = 557.2 | ¹H NMR (DMSO-d₆, 400 |
| | | MHz) δ 11.60 (s, 1H), 9.00 (s, 1H), 8.35- |
| | | 8.29 (m, 1H), 8.18 (s, 1H), 8.20-8.16 |
| | | (m, 1H), 8.00 (s, 1H), 7.87 (d, J = 2.6 |
| | | Hz, 1H), 7.80 (d, J = 8.4 Hz, 1H), 7.58- |
| | | 7.52 (m, 1H), 7.23 (s, 1H), 7.04 (s, 1H), |
| | | 6.88-6.77 (m, 1H), 6.12 (d, J = 2.4 Hz, |
| | | 1H), 5.68-5.63 (m, 1H), 5.41 (s, 2H), |
| | | 4.54 (s, 1H), 4.14 (m, 1H), 3.52 (m, 1H), |
| | | 3.24 (m, 1H), 2.90-2.78 (m, 1H), 2.03 |
| | | (m, 2H), 1.50-1.37 (m, 2H) |
| 113 | [M + H]⁺ = 557.1 | ¹H NMR (CD₃OD, 400 MHZ) δ 8.83 (d, |
| | | J = 5.6 Hz, 1H), 8.16 (s, 1H), 7.98 (s, 1H), |
| | | 7.76 (d, J = 2.8 Hz, 1H), 7.67 (d, J = 4.8 |
| | | Hz, 1H), 7.45 (dd, J = 2.8, 8.8 Hz, 1H), |
| | | 7.20 (d, J = 8.8 Hz, 1H), 7.02 (s, 1H), |
| | | 6.81 (dd, J = 10.8, 16.8 Hz, 1H), 6.20 |
| | | (dd, J = 2.0, 16.8 Hz, 1H), 5.74 (dd, J = |
| | | 2.0, 10.8 Hz, 1H), 5.38 (s, 2H), 4.74- |
| | | 4.65 (m, 1H), 4.22 (m, 1H), 3.40 (m, 1H), |
| | | 3.36 (m, 1H), 3.00-2.88 (m, 1H), 2.26- |
| | | 2.13 (m, 2H), 1.67-1.54 (m, 2H) |
| 114 | [M + H]⁺ = 490.2 | ¹H NMR (DMSO-d₆, 400 |
| | | MHz) δ 11.60 (br, 1H), 9.20 (s, 1H), |
| | | 8.88 (d, J = 5.2 Hz, 1H), 8.18 (s, 1H), |
| | | 8.00 (s, 1H), 7.91-7.85 (m, 1H), 7.67 |
| | | (d, J = 5.2 Hz, 1H), 7.59-7.48 (m, 1H), |
| | | 7.27-7.15 (m, 1H), 7.05 (s, 1H), 6.88- |
| | | 6.75 (m, 1H), 6.16-6.02 (m, 1H), 5.70- |
| | | 5.60 (m, 1H), 5.33 (s, 2H), 4.63-4.46 |
| | | (m, 1H), 4.19-4.07 (m, 1H), 3.61-3.49 |
| | | (m, 1H), 3.27-3.22 (m, 1H), 2.84 (m, |
| | | 1H), 2.09-1.94 (m, 2H), 1.53-1.36 (m, |
| | | 2H) |
| 116 | [M + H]⁺ = 490.2 | ¹H NMR (DMSO-d₆, 400 |
| | | MHz) δ 11.65-11.55 (m, 1H), 8.86 (s, |
| | | 1H), 8.73-8.61 (m, 2H), 8.19 (s, 1H), |
| | | 8.00 (s, 1H), 7.85 (br d, J = 2.4 Hz, 1H), |
| | | 7.63-7.48 (m, 1H), 7.29 (d, J = 8.8 Hz, |
| | | 1H), 7.05 (s, 1H), 6.94-6.74 (m, 1H), |
| | | 6.12 (d, J = 2.0 Hz, 1H), 5.75-5.58 (m, |
| | | 1H), 5.37 (s, 2H), 4.56 (d, J = 12.8 Hz, |
| | | 1H), 4.26-4.05 (m, 1H), 3.63-3.44 (m, |
| | | 1H), 3.30-3.22 (m, 1H), 2.84 (s, 1H), |
| | | 2.09-1.98 (m, 2H), 1.44 (d, J = 11.6 Hz, |
| | | 2H) |
| 117 | [M + H]⁺ = 523.2 | ¹H NMR (DMSO-d₆, 400 |
| | | MHz) δ 11.60 (s, 1H), 8.59 (d, J = 5.2 |
| | | Hz, 1H), 8.24-8.13 (m, 1H), 8.00 (s, |
| | | 1H), 7.86 (d, J = 2.4 Hz, 1H), 7.67-7.63 |
| | | (m, 1H), 7.56-7.52 (m, 2H), 7.24 (d, J = |
| | | 9.2 Hz, 1H), 7.06-7.02 (m, 1H), 6.88- |
| | | 6.77 (m, 1H), 6.17-6.04 (m, 1H), 5.70- |
| | | 5.62 (m, 1H), 5.30 (s, 2H), 4.57-4.55 |
| | | (m, 1H), 4.15-4.13 (m, 1H), 3.54-3.50 |
| | | (m, 1H), 3.29-3.26 (m, 1H), 2.85-2.83 |
| | | (m, 1H), 2.09-1.98 (m, 2H), 1.52-1.36 |
| | | (m, 2H) |
| 118 | [M + H]⁺ = 507.2 | ¹H NMR (DMSO-d₆, 400 |
| | | MHz) δ 11.60 (br, 1H), 8.60 (br, 1H), |
| | | 8.18 (s, 1H), 7.99 (s, 1H), 7.87-7.78 (m, |
| | | 2H), 7.67 (d, J = 4.6 Hz, 1H), 7.54 (d, J = |
| | | 8.8 Hz, 1H), 7.23 (d, J = 9.0Hz, 1H), |
| | | 7.04 (s, 1H), 6.88-6.77 (m, 1H), 6.10 |
| | | (d, J = 16.8 Hz, 1H), 5.66 (d, J = 10.8 |
| | | Hz, 1H), 5.28 (s, 2H), 4.56 (m, 1H), 4.14 |
| | | (m, 1H), 3.57-3.47 (m, 1H), 3.28-3.23 |
| | | (m, 1H), 2.83 (m, 1H), 2.02 (m, 2H), 1.45 |
| | | (m, 2H) |
| 119 | [M + H]⁺ = 495.1 | ¹H NMR (DMSO-d₆, 400 |
| | | MHz) δ 11.60 (s, 1H), 9.15 (d, J = 2.0 |
| | | Hz, 1H), 8.21-8.16 (m, 1H), 7.98 (s, |
| | | 1H), 7.86-7.77 (m, 2H), 7.60-7.52 (m, |
| | | 1H), 7.30 (d, J = 8.8 Hz, 1H), 7.04 (s, |
| | | 1H), 6.89-6.77 (m, 1H), 6.16-6.06 (m, |
| | | 1H), 5.70-5.62 (m, 1H), 5.32 (s, 2H), |
| | | 4.56 (m, 1H), 4.14 (m, 1H), 3.52 (m, 1H), |
| | | 3.29-3.22 (m, 1H), 2.84 (m, 1H), 2.02 |
| | | (m, 2H), 1.53-1.35 (m, 2H) |
| 120 | [M + H]⁺ = 490.2 | ¹H NMR (DMSO-d₆, 400 |
| | | MHz) δ 11.62 (s, 1H), 9.24 (dd, J1 = 2.0 |
| | | Hz, J2 = 4.8 Hz, 1H), 8.18 (s, 1H), 8.02 |
| | | (s, 1H), 7.87-7.85 (m, 2H), 7.82 (d, J = |
| | | 4.8 Hz, 1H), 7.60-7.52(m, 1H), 7.29 (d, |
| | | J = 8.8 Hz, 1H), 7.05 (s, 1H), 6.83 (dd, |
| | | J1 = 10.4 Hz, J2 = 16.8 Hz, 1H), 6.10 |
| | | (dd, J1 = 2.8 Hz, J2 = 16.8 Hz, 1H), 5.66 |
| | | (dd, J1 = 2.4 Hz, J2 = 10.4 Hz, 1H), 5.49 |
| | | (s, 2H), 4.55 (m, 1H), 4.14 (m, 1H), 3.52 |
| | | (m, 1H), 3.30-3.26 (m, 1H), 2.83 (m, |
| | | 1H), 2.04-2.01 (m, 2H), 1.48-1.39 (m, |
| | | 2H) |
| 121 | [M + H]⁺ = 489.2 | ¹H NMR (DMSO-d₆, 400 |
| | | MHz) δ 11.60 (br, 1H), 8.70 (d, J = 1.8 |
| | | Hz, 1H), 8.58-8.53 (m, 1H), 8.18 (s, |
| | | 1H), 7.98 (s, 1H), 7.93-7.87 (m, 1H), |
| | | 7.86-7.80 (m, 1H), 7.59-7.52 (m, 1H), |
| | | 7.48-7.42 (m, 1H), 7.26 (d, J = 9.0 Hz, |
| | | 1H), 7.04 (s, 1H), 6.88-6.77 (m, 1H), |
| | | 6.15-6.05 (m, 1H), 5.69-5.61 (m, 1H), |
| | | 5.26 (s, 2H), 4.59-4.51 (m, 1H), 4.18- |
| | | 4.09 (m, 1H), 3.56-3.46 (m, 1H), 3.27 |
| | | (m, 1H), 2.89-2.78 (m, 1H), 2.03 (m, |
| | | 2H), 1.50-1.37 (m, 2H) |
| 126 | [M + H]⁺ = 523.1 | ¹H NMR (DMSO-d₆, 400 MHZ) δ 11.60 |
| | | (br, 1 H) 8.66 (d, J = 2.40 Hz, 1 H) 8.18 |
| | | (s, 1 H) 7.95-8.09 (m, 2 H) 7.79-7.91 |
| | | (m, 1 H) 7.62 (d, J = 8.44 Hz, 1 H) 7.50- |
| | | 7.58 (m, 1 H) 7.21 (d, J = 8.80 Hz, 1 H) |
| | | 7.04 (m, 1 H) 6.83 (dd, J = 16.80, 10.40 |
| | | Hz, 1 H) 6.10 (dd, J = 16.80, 2.40 Hz, 1 |
| | | H) 5.66 (dd, J = 10.40, 2.40 Hz, 1 H) |
| | | 5.29 (s, 2 H) 4.56 (m, 1 H) 4.14 (m, 1 H) |
| | | 3.47-3.55 (m, 1 H) 3.23-3.28 (m, 1 H) |
| | | 2.83 (m, 1 H) 1.99-2.07 (m, 2 H) 1.39- |
| | | 1.50 (m, 2 H) |
| 127 | [M + H]⁺= 489.4 | ¹H NMR (CD₃OD, 400 |
| | | MHz) δ 8.61-8.52 (m, 2H), 8.15 (s, |
| | | 1H), 7.74 (d, J = 2.4 Hz, 1H), 7.60 (d, J = |
| | | 6.0 Hz, 2H), 7.46-7.40 (m, 1H), 7.14 |
| | | (d, J = 8.8 Hz, 1H), 7.02 (s, 1H), 6.87- |
| | | 6.72 (m, 1H), 6.26-6.12 (m, 1H), 5.80- |
| | | 5.66 (m, 1H), 5.30 (s, 2H), 4.69 (m, 1H), |
| | | 4.29-4.17 (m, 1H), 3.39 (m, 2H), 3.00- |
| | | 2.89 (m, 1H), 2.25-2.13 (m, 2H), 1.68- |
| | | 1.52 (m, 2H) |
| 128 | [M + H]⁺ = 490.4 | ¹H NMR (CD₃OD, 400 MHZ) δ 8.83 (d, |
| | | J = 5.2 Hz, 2H), 8.15 (s, 1H), 7.70 (d, J = |
| | | 2.6 Hz, 1H), 7.46 (t, J = 5.2 Hz, 1H), |
| | | 7.40-7.33 (m, 1H), 7.10 (d, J = 8.8 Hz, |
| | | 1H), 7.01 (s, 1H), 6.87-6.75 (m, 1H), |
| | | 6.24-6.15 (m, 1H), 5.77-5.70 (m, 1H), |
| | | 5.36 (s, 2H), 4.69 (m, 1H), 4.22 (m, 1H), |
| | | 3.40-3.33 (m, 1H), 3.00-2.86 (m, 1H), |
| | | 2.17 (m, 2H), 1.67-1.49 (m, 2H) |
| 153 | [M + H]⁺ = 495.1 | ¹H NMR (DMSO-d₆, 400 |
| | | MHz) δ 11.88-11.45 (m, 1H), 8.19 (d, J = |
| | | 2.0 Hz, 1H), 8.01 (s, 1H), 7.92-7.84 |
| | | (m, 2H), 7.80 (d, J = 3.2 Hz, 1H), 7.61- |
| | | 7.52 (m, 1H), 7.29 (d, J = 9.2 Hz, 1H), |
| | | 7.05 (s, 1H), 6.83 (dd, J = 10.4, 16.8 Hz, |
| | | 1H), 6.10 (dd, J = 2.4, 16.8 Hz, 1H), |
| | | 5.66 (dd, J = 2.4, 10.4 Hz, 1H), 5.54 (s, |
| | | 2H), 4.56 (m, 1H), 4.19-4.10 (m, 1H), |
| | | 3.58-3.47 (m, 1H), 3.29-3.21 (m, 1H), |
| | | 2.88-2.80 (m, 1H), 2.07-1.99 (m, 2H), |
| | | 1.49-1.39 (m, 2H) |
| 156 | [M + H]⁺ = 489.3 | ¹H NMR (DMSO-d₆, 400 |
| | | MHz) δ 11.60 (s, 1H), 8.53 (s, 1H), 8.60- |
| | | 8.59 (m, 1H), 8.18 (s, 1H), 7.98 (s, 1H), |
| | | 7.90-7.84 (m, 1H), 7.57 (d, J = 7.6 Hz, |
| | | 1H), 7.53 (dd, J1 = 2.4 Hz, J2 = 6.4 Hz, |
| | | 1H), 7.36 (dd, J1 = 4.8 Hz, J2 = 6.4 Hz, |
| | | 1H), 7.22 (d, J = 8.8 Hz, 1H), 7.04 (d, J = |
| | | 0.8 Hz, 1H), 6.83 (dd, J1 = 10.4 Hz, J2 = |
| | | 16.8 Hz, 1H), 6.10 (dd, J1 = 2.4 Hz, J2 = |
| | | 24.8 Hz, 1H), 5.66 (dd, J1 = 2.4 Hz, J2 = |
| | | 10.4 Hz, 1H), 5.28 (s, 2H), 4.56 (m, |
| | | 1H), 4.14 (m, 1H), 3.51 (m, 1H), 3.32- |
| | | 3.27 (m, 1H), 2.83 (m, 1H), 2.04-2.02 |
| | | (m, 2H), 1.45-1.42 (m, 2H) |

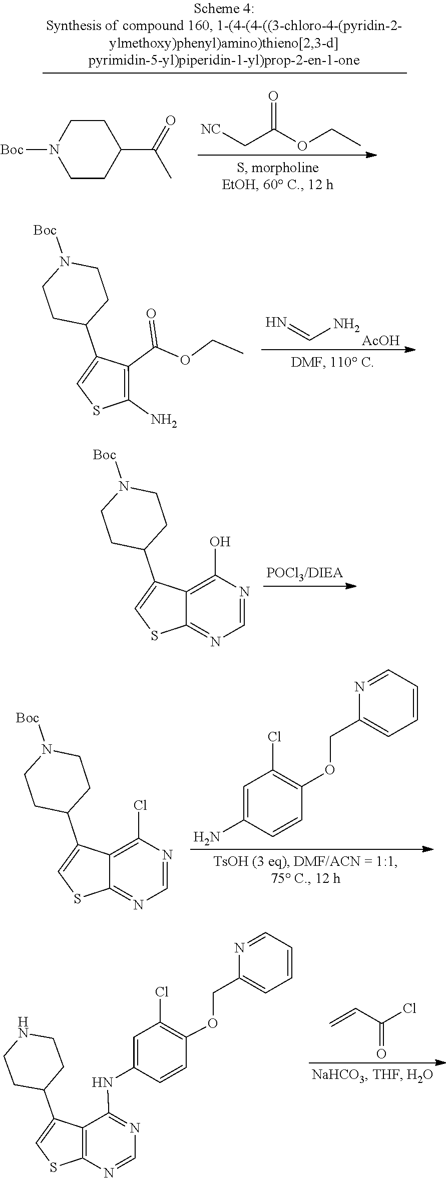

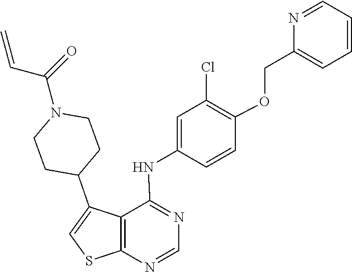 [SMILES: C=CC(=O)N1CCC(c2csc3ncnc(Nc4ccc(OCc5ccccn5)c(Cl)c4)c23)CC1]

Compounds 158, 159, 160, 162, and 163 can be prepared via a similar procedure to the one shown in Scheme 4.

TABLE 5
Characterization of Compounds

| Com- | | |
|---|---|---|
| pound | | |
| No. | LC-MS | ¹H NMR |
| 160 | [M + H]⁺ = 506.2. | ¹H NMR (DMSO-d₆, 400 MHZ) δ 8.64- |
| | | 8.57 (m, 1H), 8.42 (s, 1H), 8.38 (s, 1H), |
| | | 7.91-7.85 (m, 1H), 7.80-7.76 (m, 1H), |
| | | 7.58 (d, J = 7.8 Hz, 1H), 7.51-7.45 (m, |
| | | 1H), 7.40 (s, 1H), 7.39-7.34 (m, 1H), |
| | | 7.25 (d, J = 8.8 Hz, 1H), 6.91-6.75 (m, |
| | | 1H), 6.16-6.04 (m, 1H), 5.70-5.63 (m, |
| | | 1H), 5.31 (s, 2H), 4.60-4.56 (m, 1H), |
| | | 4.19-4.15 (m, 1H), 3.75-3.68 (m, 1H), |
| | | 3.30-3.23 (m, 1H), 2.88 -2.82 (m, 1H), |
| | | 2.11-2.08 (m, 2H), 1.58-1.41 (m, 2H) |
| 161 | [M + H]⁺ = 587.2. | ¹H NMR (DMSO-d₆, 400 MHZ) δ 10.54 (s, |
| | | 1H), 8.43-8.36 (m, 2H), 8.24 (d, J = 5.6 |
| | | Hz, 1H), 7.84-7.81 (m, 1H), 7.78 (d, J = |
| | | 2.4 Hz, 1H), 7.44 (s, 1H), 7.31 (dd, J = |
| | | 2.4, 10.8 Hz, 1H), 7.11 (dd, J = 2.0, 8.8 |
| | | Hz, 1H), 6.90-6.80 (m, 1H), 6.77 (dd, J = |
| | | 2.4, 5.8 Hz, 1H), 6.11 (dd, J = 2.4, 16.8 |
| | | Hz, 1H), 5.71-5.59 (m, 1H), 4.66-4.53 |
| | | (m, 1H), 4.28-4.15 (m, 1H), 3.62-3.75 |
| | | (m, 1H), 3.32-3.30 (m, 1H), 2.90-2.88 |
| | | (m, 2H), 2.32-2.31 (m, 2H), 1.82-1.80 (m, |
| | | 2H), 1.63-1.62 (m, 4H), 1.57-1.46 (m, |
| | | 4H) |

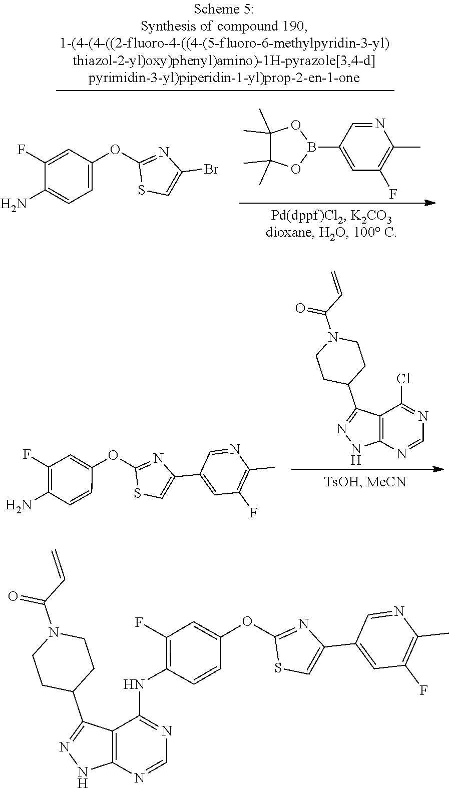

Compounds 149, 188, 189, 190, 191, and 206 can be prepared via a similar procedure to the one shown in Scheme 5.

TABLE 6
Characterization of Compounds

| Com- | | |
|---|---|---|
| pound | | |
| No. | LC-MS | ¹H NMR |
| 149 | [M + H]⁺ = 573.3 | ¹H NMR (CD₃OD, 400 MHZ) δ 8.98 (s, |
| | | 2H), 8.31-8.17 (m, 2H), 7.52 (s, 1H), |
| | | 7.39 (dd, J = 2.8, 11.2 Hz, 1H), 7.32- |
| | | 7.24 (m, 1H), 7.06 (s, 1H), 6.82 (dd, J = |
| | | 10.8, 16.8 Hz, 1H), 6.21 (dd, J = 2.0, 16.8 |
| | | Hz, 1H), 5.75 (dd, J = 2.0, 10.8 Hz, 1H), |
| | | 4.79-4.69 (m, 1H), 4.27 (m, 1H), 4.05 (s, |
| | | 3H), 3.43-3.35 (m, 2H), 3.04-2.90 (m, |
| | | 1H), 2.32-2.18 (m, 2H), 1.65 (dq, J = |
| | | 3.6, 12.4 Hz, 2H) |
| 188 | [M + H]⁺ = 611.2 | ¹H NMR (DMSO-d₆, 400 MHZ) δ = 13.39 |
| | | (br, 1H), 9.25 (s, 1H), 8.70 (s, 1H), 8.45 |
| | | (d, J = 8.6 Hz, 1H), 8.25 (s, 1H), 8.10 (s, |
| | | 1H), 7.99 (d, J = 8.4 Hz, 1H), 7.78-7.71 |
| | | (m, 1H), 7.66-7.60 (m, 1H), 7.42 (d, J = |
| | | 8.6 Hz, 1H), 6.90-6.82 (m, 1H), 6.16- |
| | | 6.08 (m, 1H), 5.70-5.65 (m, 1H), 5.71- |
| | | 5.64 (m, 1H), 4.51 (m, 1H), 4.17 (m, 1H), |
| | | 3.72 (m, 1H), 3.30-3.26 (m, 1H), 2.96- |
| | | 2.87 (m, 1H), 2.12-2.03 (m, 2H), 1.73- |
| | | 1.60 (m, 2H) |
| 189 | [M + H]⁺ = 573.2 | ¹H NMR (DMSO-d₆, 400 MHZ) δ 13.39 |
| | | (br, 1H), 8.70 (s, 1H), 8.65 (d, J = 2.2 Hz, |
| | | 1H), 8.25 (s, 1H), 8.16-8.08 (m, 1H), |
| | | 7.77-7.69 (m, 1H), 7.66 (s, 1H), 7.63- |
| | | 7.57 (m, 1H), 7.42-7.36 (m, 1H), 6.93- |
| | | 6.80 (m, 2H), 6.18-6.06 (m, 1H), 5.71- |
| | | 5.64 (m, 1H), 4.50 (m, 1H), 4.21-4.11 |
| | | (m, 1H), 3.88 (s, 3H), 3.78-3.65 (m, 1H), |
| | | 3.28 (m, 1H), 2.91 (m, 1H), 2.11-2.03 |
| | | (m, 2H), 1.72-1.58 (m, 2H) |
| 190 | [M + H]⁺ = 575.2 | ¹H NMR (DMSO-d₆, 400 MHZ) δ = 13.39 |
| | | (br, 1H), 8.80 (s, 1H), 8.70 (s, 1H), 8.25 |
| | | (s, 1H), 8.03 (d, J = 10.8 Hz, 1H), 7.90 (s, |
| | | 1H), 7.79-7.70 (m, 1H), 7.62 (dd, J = |
| | | 2.4, 10.4 Hz, 1H), 7.41 (d, J = 8.8 Hz, |
| | | 1H), 6.86 (dd, J = 10.8, 16.8 Hz, 1H), |
| | | 6.11 (dd, J = 2.4, 16.8 Hz, 1H), 5.68 (dd, |
| | | J = 2.4, 10.4 Hz, 1H), 4.57-4.46 (m, 1H), |
| | | 4.25-4.15 (m, 1H), 3.77-3.67 (m, 1H), |
| | | 3.33-3.25 (m, 1H), 2.90-2.80 (m, 1H), |
| | | 2.47 (s, 3H), 2.13-2.01 (m, 2H), 1.73 - |
| | | 1.58 (m, 2H) |

### Preparation of Intermediates

### Preparation of tert-Butyl 4-(4-chlorothieno[2,3-d]pyrimidin-5-yl)piperidine-1-carboxylate, Int-1

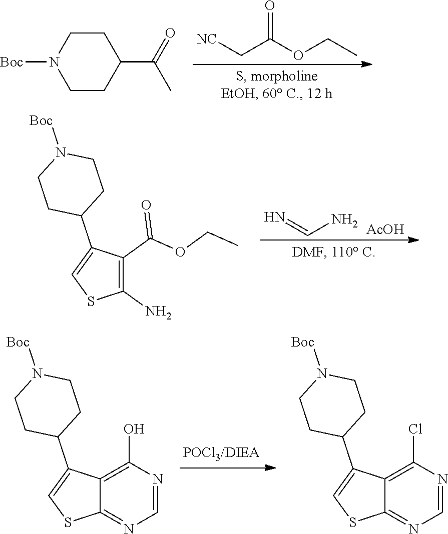

### Step 1: Preparation of tert-Butyl 4-(5-amino-4-ethoxycarbonyl-3-thienyl) piperidine-1-carboxylate

A mixture of tert-Butyl 4-acetylpiperidine-1-carboxylate (10 g, 43.99 mmol, 1 eq), ethyl 2-cyanoacetate (4.98 g, 43.99 mmol, 4.69 mL, 1 eq), morpholine (3.83 g, 43.99 mmol, 3.87 mL, 1 eq), S (14.16 g, 441.60 mmol, 10.04 eq) was added in EtOH (100 mL) at 20° C. The mixture was stirred at 80° C. for 12 h. LC-MS demonstrated full consumption of tert-Butyl 4-acetylpyridine-1-carboxylate and desired mass was detected. The reaction mixture was filtered and the filtrate was concentrated to give the residue which was purified by column chromatography (PE/EA-O/1 to 5/1) to give tert-Butyl 4-(5-amino-4-ethoxycarbonyl-3-thienyl)piperidine-1-carboxylate (4 g, 10.27 mmol, 23.34% yield, 91% purity) as a yellow solid. LC-MS: [M-Boc+H]⁺=255.1, ¹H NMR (DMSO-d₆, 400 MHz) δ 7.39-7.27 (m, 2H), 5.98 (s, 1H), 4.22-4.15 (m, 2H), 4.05-4.01 (m, 2H), 3.15-3.05 (m, 1H), 2.80-2.68 (m, 2H), 1.85-1.78 (m, 2H), 1.40 (s, 9H), 1.32-1.23 (in, 5H).

### Step 2: Preparation of tert-Butyl 4-(4hydroxythieno[2,3-d]pyrimidin-5-yl)piperidine-1-carboxylate

To a solution of tert-Butyl 4-(5-amino-4-ethoxycarbonyl-3-thienyl)piperidine-1-carboxylate (4 g, 10.27 mmol, 91% purity, 1 eq) in DMF (40 mL) was added acetic acid and formamide acetate (2.14 g, 20.54 mmol, 2 eq). The mixture was stirred at 110° C. for 12 h. LC-MS demonstrated full consumption of tert-Butyl 4-(5-amino-4-ethoxycarbonyl-3-thienyl) piperidine-1-carboxylate and the desired mass was detected. The reaction mixture was poured into ice water 200 mL, extracted with EA (100 mL×2). The combined organic layers were washed with water (100 mL×2), dried over Na₂SO₄, filtered and concentrated under reduced pressure to give a residue. The residue was triturated with PE/EA-5/1 (30 mL) for 0.5 h. The resulting mixture was filtered and the filter cake was collected and dried under reduced pressure to give tert-Butyl 4-(4-hydroxythieno[2,3-d]pyrimidin-5-yl)piperidine-1-carboxylate (2.5 g, 6.78 mmol, 66.05% yield, 91% purity) as an off-white solid. LC-MS: [M-Boc+H]⁺=236.2, ¹H NMR (DMSO-d₆, 400 MHz) δ 12.42 (s, 1H), 8.07 (s, 1H), 7.25 (s, 1H), 4.08-4.05 (m, 2H), 3.46-3.37 (m, 1H), 2.91-2.70 (m, 2H), 1.93-1.89 (m, 2H), 1.49-1.38 (m, 11H).

### Step 3: Preparation of tert-Butyl 4-(4-chlorothieno[2,3-d]pyrimidin-5-yl)piperidine-1-carboxylate

To a solution of tert-Butyl 4-(4-hydroxythieno[2,3-d]pyrimidin-5-yl)piperidine-1-carboxylate (0.8 g, 2.17 mmol, 91% purity, 1 eq) in toluene (20 mL) was added DIEA (5.61 g, 43.41 mmol, 7.56 mL, 20 eq) and POCl₃ (990.00 mg, 6.46 mmol, 0.6 mL, 2.97 eq) at 0° C. The mixture was stirred at 100° C. for 12 h under N₂ atmosphere. TLC (PE/EA=1/1) showed full consumption of tert-Butyl 4-(4-hydroxythieno [2,3-d]pyrimidin-5-yl)piperidine-1-carboxylate and a new spot was formed. The reaction mixture was concentrated under reduced pressure to give a residue which was further purified by column chromatography (SiO₂, Petroleum ether/Ethyl acetate=1/0 to 5/1) to afford tert-Butyl 4-(4-chlorothieno [2,3-d]pyrimidin-5-yl)piperidine-1-carboxylate (1.5 g, 4.04 mmol, 93.16% yield, 95.4% purity) as yellow oil. ¹H NMR (DMSO-d₆, 400 MHz) δ8.91 (s, 1H), 7.87 (s, 1H), 4.12-4.09 (m, 2H), 3.59-3.49 (m, 1H), 2.90-2.80 (m, 2H), 2.01-1.97 (m, 2H), 1.60-1.47 (m, 2H), 1.42 (s, 9H).

### Preparation of 1-(4-(4-chloro-7H-pyrrolo[2,3-d]pyrimidin-5-yl)piperidin-1-yl)prop-2-en-1-one, Int-2

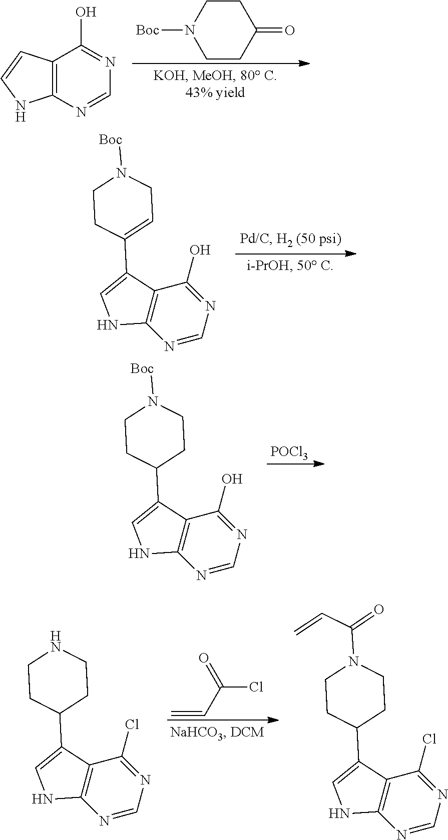

### Step 1: Preparation of tert-Butyl 4-(4-hydroxy-7H-pyrrolo[2,3-d]pyrimidin-5-yl)-3,6-dihydro-2H-pyridine-1-carboxylate

To a solution of 7H-pyrrolo[2,3-d]pyrimidin-4-ol (100 g, 740.07 mmol, 1 eq) and tert-Butyl 4-oxopiperidine-1-carboxylate (294.91 g, 1.48 mol, 2 eq) in MeOH (1000 mL) was added KOH (249.13 g, 4.44 mol, 6 eq) at 20° C. The reaction mixture was stirred at 80° C. for 72 hours. LC-MS analysis indicated full consumption of 7H-pyrrolo[2,3-d]pyrimidin-4-ol and the desired mass was detected. The reaction was quenched with water (1000 mL) and was neutralized with HCl (1 M). The resulting mixture was filtered to give a solid as the crude product which was further purified by reversed-phase HPLC (0.1% FA condition) to afford tert-Butyl 4-(4-hydroxy-7H-pyrrolo[2,3-d]pyrimidin-5-yl)-3,6-dihydro-2H-pyridine-1-carboxylate (50 g, 316.10 mmol, 21.36% yield) was a white solid. LC-MS: [M-Boc+H]⁺=217.0, ¹H NMR (DMSO-d₆, 400 MHz) δ 8.18 (s, 1H), 7.74 (d, J=1.2 Hz, 1H), 6.83 (d, J=1.2 Hz, 1H), 4.25-4.22 (m, 2H), 2.91-2.84 (m, 3H), 2.00-1.97 (m, 2H), 1.68-1.64 (m, 2H), 1.49 (s, 9H).

### Step 2: Preparation of tert-Butyl 4-(4-hydroxy-7H-pyrrolo[2,3-d]pyrimidin-5-yl)piperidine-1-carboxylate

To a solution of tert-Butyl 4-(4-hydroxy-7H-pyrrolo[2,3-d]pyrimidin-5-yl)-3,6-dihydro-2H-pyridine-1-carboxylate (25 g, 79.03 mmol, 1 eq) in f-PrOH (1200 mL) was added Pd/C (20 g, 10% Pd), the mixture was stirred at 50° C. under H₂ (50 psi) for 20 hours. LC-MS analysis indicated full consumption of tert-Butyl 4-(4-hydroxy-7H-pyrrolo[2,3-d]pyrimidin-5-yl)-3,6-dihydro-2H-pyridine-1-carboxylate and the desired mass was detected. The reaction mixture was filtered and the filtrate was concentrated under vacuum to give tert-Butyl 4-(4-hydroxy-7H-pyrrolo[2,3-d]pyrimidin-5-yl)piperidine-1-carboxylate (25 g, 314.10 mmol, 99.37% yield, 100% purity) as a white solid without further purification. LC-MS: [M-Boc+H]⁺=219.0, ¹H NMR (DMSO-d₆, 400 MHz) δ 11.88 (s, 1H), 11.78 (d, J=2.8 Hz, 1H), 7.82-7.81 (m, 1H), 7.05 (d, J=2.0 Hz, 1H), 6.96-6.95 (m, 1H), 3.96-3.95 (m, 2H), 3.50 (d, J=5.2 Hz, 2H), 2.92 (m, 1H), 2.44-2.43 (m, 2H), 1.42 (s, 9H).

### Step 3: Preparation of 4-chloro-5-(piperidin-4-yl)-7H-pyrrolo[2,3-d]pyrimidine

A mixture of tert-Butyl 4-(4-hydroxy-7H-pyrrolo[2,3-d]pyrimidin-5-yl)piperidine-1-carboxylate (30 g, 94.23 mmol, 1 eq) and POCl₃ (742.50 g, 4.84 mol, 450.00 mL, 51.39 eq) was stirred at 80° C. under N₂ for 12 hours. The reaction mixture was concentrated to give a residue which was diluted with THF (500 mL) and water, then neutralized with NaHCO₃ (solid) till pH reaches 7. 4-chloro-5-(4-piperidyl)-7H-pyrrolo [2,3-d]pyrimidine (15 g, crude) was obtained as a brown liquid and used into next step without further purification. LC-MS: [M+H]⁺=237.0.

### Step 4: Preparation of 1-(4-(4-chloro-7H-pyrrolo[2,3-d]pyrimidin-8-yl)piperidin-1-yl)prop-2-en-1-one

To a solution of 4-chloro-5-(4-piperidyl)-7H-pyrrolo[2,3-d]pyrimidine (74 g, 312.63 mmol, 1 eq) in water (2 L) and THF (2 L), NaHCO₃ (262.64 g, 3.13 mol, 121.59 mL, 10 eq) was added. Prop-2-enoyl chloride (40 g, 441.95 mmol, 36.04 mL, 1.41 eq) was added drop-wise. The mixture was stirred at 20° C. for 0.5 h. TLC (PE:EA=0:1) showed that 4-chloro-5-(4-piperidyl)-7H-pyrrolo[2,3-d]pyrimidine was consumed and one new spot was formed. LC-MS analysis indicated full consumption of 4-chloro-5-(4-piperidyl)-7H-pyrrolo[2,3-d]pyrimidine and the desired mass was detected. The reaction mixture was extracted with EA (500 mL×3). The organic layers were combined, washed with brine (30 mL), dried over anhydrous Na₂SO₄, filtered and concentrated under vacuum to give a residue which was further purified by column chromatography to afford 1-[4-(4-chloro-7H-pyrrolo[2,3-d]pyrimidin-5-yl)-1-piperidyl]prop-2-en-1-one (48 g, 162.12 mmol, 51.86%, 98.2% purity) as a light yellow solid. LC-MS: [M+H]⁺=291.3, ¹H NMR (DMSO-d₆, 400 MHz) δ 12.37 (s, 1H), 8.53 (s, 1H), 7.49 (d, J=2.4 Hz, 1H), 6.84 (dd, J=10.4 Hz, 14.6 Hz, 1H), 6.84 (dd, J=2.4 Hz, 16.4 Hz, 1H), 5.67 (dd, J=2.4 Hz, 10.4 Hz, 1H), 4.59 (d, J=12.8 Hz, 1H), 4.18 (d, J=12.8 Hz, 1H), 3.33-3.00 (m, 1H), 3.20 (t, J=2.8 Hz, 1H), 2.76 (t, J=2.8 Hz, 1H), 2.03-1.98 (m, 2H), 1.56-1.47 (m, 2H).

### Preparation of 1-(4-(4-chloro-1H-pyrazolo[3,4-d]pyrimidin-3-yl)piperidin-1-yl)prop-2-en-1-one, Int-3

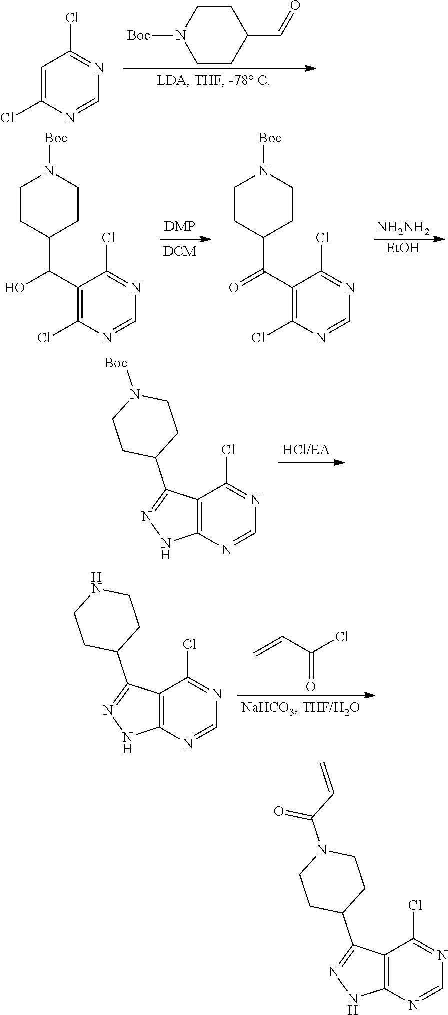

### Step 1: Preparation of tert-Butyl 4-((4,6-dichloropyrimidin-5-yl)hydroxy)methyl)piperidine-1-carboxylate

A solution of LDA (2 M, 17.62 mL, 1.05 eq) in THF (80 mL) was pre-cooled to −60° C. and 4,6-dichloropyrimidine (5 g, 33.56 mmol, 1 eq) was added. The mixture was stirred at −60° C. for 30 min, and then a solution of tert-Butyl 4-formylpiperidine-1-carboxylate (7.16 g, 33.56 mmol, 1 eq) in THF (20 mL) was added dropwise at the same temperature. The reaction mixture was stirred at −60° C. for 30 min. LC-MS analysis indicated full consumption of the reactant and mass of the desired compound was detected. The mixture was quenched with saturation NH₄Cl solution (200 mL), extracted with ethyl acetate (200 mL). The combined organic layers were washed by brine (200 mL), dried over anhydrous sodium sulfate and concentrated under reduced pressure to give a residue which was further purified by column chromatography (SiO₂, Petroleum ether/Ethyl acetate=I/O to 3/1) to give tert-Butyl 4-[(4,6-dichloropyrimidin-5-yl)-hydroxy-methyl]piperidine-1-carboxylate (3.2 g, 7.91 mmol, 23.58% yield, 89.57% purity) as a brown oil. ¹H NMR: (400 MHz, CDCl₆) δ 8.71 (s, 1H), 5.05 (br t, J=8.0 Hz, 1H), 4.24-4.21 (m, 1H), 4.15-4.09 (m, 1H), 2.79-2.54 (m, 3H), 2.40-2.28 (m, 1H), 2.23-2.19 (m, 1H), 1.46 (s, 9H), 1.38-1.32 (m, 1H), 1.31-1.26 (m, 1H), LC-MS: [M-t-Bu+H]⁺=306.0.

### Step 2: Preparation of tert-Butyl 4-(4,6-dichloropyrimidine-5-carbonyl)piperidine-1-carboxylate

To a solution of tert-Butyl 4-[(4,6-dichloropyrimidin-5-yl)-hydroxy-methyl]piperidine-1-carboxylate (2.9 g, 8.01 mmol, 1 eq) in DCM (100 mL) was added DMP (3.74 g, 8.81 mmol, 2.73 mL, 1.1 eq) at 0° C. The mixture was stirred at 20° C. for 30 min. TLC (PE/EA=3:1) indicated full consumption of the alcohol reactant. The mixture was diluted with ethyl acetate (100 mL), washed with saturated bicarbonate sodium solution (100 mL×3), filtered, and dried over anhydrous Na₂SO₄. The organic solution was concentrated under reduced pressure to give tert-Butyl 4-(4,6-dichloropyrimidine-5-carbonyl)piperidine-1-carboxylate (2.4 g, 5.57 mmol, 69.57% yield, 83.6% purity) as a brown solid without further purification. ¹H NMR: (400 MHz, CDCl₃) δ 8.85 (s, 1H), 4.25-4.15 (m, 2H), 3.06-3.00 (m, 1H), 2.83-2.79 (m, 2H), 1.97-1.93 (m, 2H), 1.79-1.71 (m, 2H), 1.47 (s, 9H), LC-MS: [M-t-Bu+H]⁺=304.0

### Step 3: Preparation of tert-Butyl 4-(4-chloro-1H-pyrazolo[3,4-d]pyrimidin-3-yl)piperidine-1-carboxylate

To a solution of tert-Butyl 4-(4,6-dichloropyrimidine-5-carbonyl)piperidine-1-carboxylate (1.8 g, 5.00 mmol, 1 eq) in EtOH (20 mL) was added triethyl amine (1.52 g, 14.99 mmol, 2.09 mL, 3 eq) and hydrazine hydrate (255.24 mg, 5.00 mmol, 247.81 uL, 98% purity, 1 eq) at 0° C. The mixture was stirred at 20° C. for 1 h. LC-MS analysis indicated full consumption of the ketone reactant and mass of the desired compound was detected. The reaction mixture was filtered and the filtrate was concentrated under reduced pressure to give a residue which was further purified by column chromatography (SiO₂, Petroleum ether/Ethyl acetate=1/0 to 3/1) to give tert Butyl 4-(4-chloro-1H-pyrazolo[3,4-d]pyrimidin-3-yl)piperidine-1-carboxylate (1.3 g, 3.81 mmol, 76.25% yield, 99% purity) as a white solid. ¹H NMR: (400 MHz, CDCl₃) δ 12.04 (br s, 1H), 8.81 (s, 1H), 4.35-4.25 (m, 2H), 3.53-3.46 (m, 1H), 3.00-2.93 (m, 2H), 2.14-2.06 (m, 2H), 2.00-1.84 (m, 2H), 1.50 (s, 9H), LC-MS: [M+H]⁺=282.0.

### Step 4: Preparation of 4-chloro-3-(piperidin-4-yl)-1H-pyrazolo[3,4-d]pyrimidine

To a solution of tert-Butyl 4-(4-chloro-1H-pyrazolo[3,4-d]pyrimidin-3-yl)piperidine-1-carboxylate (1.1 g, 3.26 mmol, 1 eq) in dioxane (20 mL) was added HCl/dioxane (4 M, 40 mL, 49.14 eq). The mixture was stirred at 50° C. for 2 h. TLC (PE/EA=0:1) indicated full consumption of the reactant and a new spot was detected. The mixture was concentrated under reduced pressure to give 4-chloro-3-(4-piperidyl)-1H-pyrazolo[3,4-d]pyrimidine (892 mg, 3.25 mmol, 99.92% yield, HCl) as a white solid, which was used directly for next step without further purification.

### Step 5: Preparation of 1-(4-(4-chloro-1H-pyrazolo[3,4-d]pyrimidin-3-yl)piperidin-1-yl)prop-2-en-1-one

To a solution of 4-chloro-3-(4-piperidyl)-1H-pyrazolo[3,4-d]pyrimidine (892 mg, 3.25 mmol, 1 eq, HCl) in THF (15 mL) and H₂O (15 mL) was added NaHCO₃ (1.37 g, 16.27 mmol, 632.71 uL, 5 eq) and prop-2-enoyl chloride (294.49 mg, 3.25 mmol, 265.30 uL, 1 eq) at 0° C. The reaction mixture was stirred at 20° C. for 10 min. TLC (EA) indicated full consumption of the reactant and one new spot was detected. The mixture was diluted with ethyl acetate (50 mL), washed by brine (30 ml×2), dried over anhydrous Na₂SO₄, filtered and concentrated under reduced pressure to give a residue which was triturated with 2-methoxy-2-methylpropane (5 mL) at 20° C. for 10 min. The resulting suspension was filtered to give 1-[4-(4-chloro-1H-pyrazolo[3,4-d]pyrimidin-3-yl)-1-piperidyl]prop-2-en-1-one (800 mg, 2.71 mmol, 83.44% yield, 99% purity) as a white solid. ¹H NMR: (400 MHz, CDCl₃) δ 11.71 (br s, 1H), 8.79 (s, 1H), 6.63 (dd, J=2.0, 17.6 Hz, 1H), 5.73 (dd, J=2.0, 10.4 Hz, 1H), 4.84-4.79 (m, 1H), 4.21-4.14 (m, 1H), 3.68-3.58 (m, 1H), 3.40-3.30 (m, 1H), 2.96-2.93 (m, 1H), 2.18-2.10 (m, 2H), 1.99-1.95 (m, 2H), LC-MS: [M+H]⁺=292.2.

### Preparation of 1-(4-(4-chloropyrrolo[2,1-f][1,2,4]triazin-5-yl)piperidin-1-yl)prop-2-en-1-one, Int-4

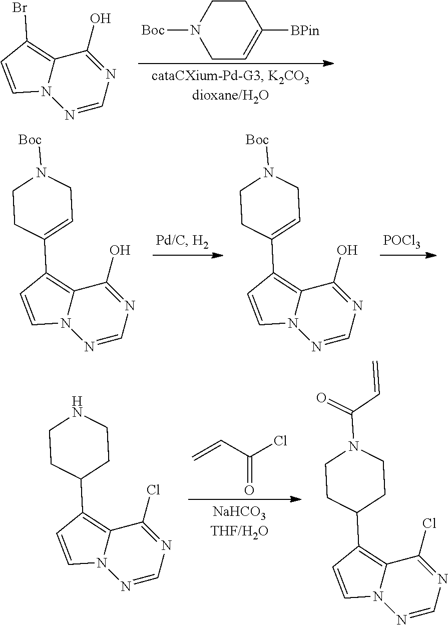

### Step 1: Preparation of tert-Butyl 4-(4-hydroxypyrrolo[2,1-f][1,2,4]triazin-5-yl)-3,6-dihydropyridine-1(2H)-carboxylate

To a solution of 5-bromopyrrolo[2,1-f][1,2,4]triazin-4-ol (5 g, 23.36 mmol, 1 eq) and tert-Butyl4-(4,4,5,5-tetramethyl-1,3,2-dioxaborolan-2-yl)-3,6-dihydro-2H-pyridine-1-carboxylate (8.67 g, 28.03 mmol, 1.2 eq) in dioxane (100 mL) and H₂O (5 mL) were added [2-(2-aminophenyl)phenyl]palladium(1+); bis(1-adamantyl)-Butyl-phosphane; methanesulfonate (3.40 g, 4.67 mmol, 0.2 eq) and K₂CO₃ (9.69 g, 70.09 mmol, 3 eq). The reaction mixture was stirred at 100° C. for 12 h under N₂ atmosphere. LC-MS analysis indicated full consumption of the bromide reactant and mass of the desired compound was detected. The mixture was diluted with ethyl acetate (300 mL), washed with brine (300 mL×3), dried over anhydrous sodium sulfate, concentrated under reduced pressure to give a residue which was triturated by 2-methoxy-2-methylpropane (50 mL) at 20° C. for 2 h to give tert-Butyl4-(4-hydroxypyrrolo[2,1-f][1,2,4]triazin-5-yl)-3,6-dihydro-2H-pyridine-1-carboxylate (7 g, 21.84 mmol, 93.46% yield, 98.68% purity) as a gray solid. ¹H NMR (CDCl₃. 400 MHz,) δ 10.10 (br s, 1H), 7.50 (d, J=3.6 Hz, 1H), 7.39 (d, J=2.8 Hz, 1H), 6.51 (d, J=2.8 Hz, 1H), 6.31 (br s, 1H), 4.11 (br s, 2H), 3.66 (t, J=5.2 Hz, 2H), 2.65 (br s, 2H), 1.50 (s, 9H), LC-MS: [M-Boc+H]⁺=217.1.

### Step 2: Preparation of tert-Butyl 4-(4-hydroxypyrrolo[2,1-f][1,2,4]triazin-5-yl)piperidine-1-carboxylate

To a solution of tert-Butyl 4-(4-hydroxypyrrolo[2,1-f][1,2,4]triazin-5-yl)-3,6-dihydro-2H-pyridine-1-carboxylate (5 g, 15.81 mmol, 1 eq) in EA (100 mL) was added Pd/C (1 g, 10%). The reaction mixture was stirred at 50° C. for 12 h under H₂ (50 Psi). LC-MS indicated full consumption of the reaction and mass of desired compound was detected. The reaction mixture was filtered and the filtrate was concentrated under reduced pressure to give tert-Butyl 4-(4-hydroxypyrrolo[2,1-f][1,2,4]triazin-5-yl)piperidine-1-carboxylate (5 g, 15.47 mmol, 97.88% yield, 98.5% purity) as a white solid without further purification. ¹H NMR (CDCl₃. 400 MHz,) δ 10.56 (br s, 1H), 7.49 (d, J=3.6 Hz, 1H), 7.36 (d, J=2.8 Hz, 1H), 6.42 (d, J=2.8 Hz, 1H), 4.22 (br s, 2H), 3.53-3.41 (m, 1H), 2.95-2.85 (m, 2H), 1.95-1.90 (m, 2H), 1.66-1.57 (m, 2H), 1.49 (s, 9H), LC-MS: [M-Boc+H]⁺=219.1.

### Step 3: Preparation of 4-chloro-5-(piperidin-4-yl)pyrrolo[2,1-f][1,2,4]triazine

A solution of tert-Butyl 4-(4-hydroxypyrrolo[2,1-f][1,2,4]triazin-5-yl)piperidine-1-carboxylate (3 g, 9.42 mmol, 1 eq) in POCl_{b}(16.50 g, 107.61 mmol, 10 mL, 11.42 eq) was stirred at 100° C. for 2 h. TLC (PE/EA=0:1) analysis indicated full consumption of the reactant and one new spot was detected. The mixture was concentrated under reduced pressure to give 4-chloro-5-(4-piperidyl)pyrrolo[2,1-f][1,2,4]triazine (2.57 g, 9.41 mmol, 100.00% yield, HCl) as a yellow solid which was directly used for next step without further purification.

### Step 4: Preparation of 1-(4-(4-chloropyrrolo[2,1-f][1,2,4]triazin-5-yl)piperidin-1-yl)prop-2-en-1-one

To a solution of 4-chloro-5-(4-piperidyl)pyrrolo[2,1-f][1,2,4]triazine (2.57 g, 9.41 mmol, 1 eq, HCl) in THF (20 mL) and H₂O (20 mL) was added NaHCO₃ (3.95 g, 47.04 mmol, 1.83 mL, 5 eq) and prop-2-enoyl chloride (851.53 mg, 9.41 mmol, 767.15 uL, 1 eq) at 0° C. The mixture was stirred at 20° C. for 10 min. TLC (PE/EA=0:1) indicated full consumption of the reactant. The reaction mixture was diluted with ethyl acetate (50 mL), washed by brine (50 mL×2), dried over anhydrous sodium sulfate and concentrated under reduced pressure to give a residue which was further purified by column chromatography (SiO₂, Petroleum ether/Ethyl acetate=1/0 to 1/1) to give 1-[4-(4-chloropyrrolo[2,1-f][1,2,4]triazin-5-yl)-1-piperidyl]prop-2-en-1-one (820 mg, 1.96 mmol, 20.80% yield, 69.38% purity) as a yellow solid. ¹H NMR (CDCl₃. 400 MHz) δ 8.12 (s, 1H), 7.82 (d, J=2.8 Hz, 1H), 6.81 (d, J=2.8 Hz, 1H), 6.63-6.59 (m, 1H), 6.35-6.30 (m, 1H), 5.74-5.69 (m, 1H), 4.91-4.84 (m, 1H), 4.20-4.10 (m, 1H), 3.72-3.65 (m, 1H), 3.26-3.23 (m, 1H), 2.79 (br s, 1H), 1.75-1.62 (m, 4H), LC-MS: [M+H]⁺=291.1.

### Preparation of tert-Butyl (3-chloro-4-((2-chloropyridin-4-yl)oxy)phenyl)carbamate, Int-5

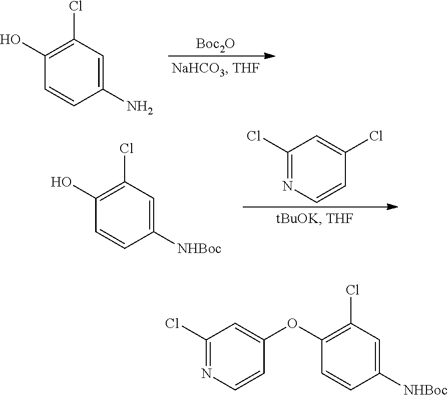

### Step 1: Preparation of tert-Butyl (3-chloro-4-hydroxyphenyl)carbamate

To a solution of 4-amino-2-chloro-phenol (10 g, 69.65 mmol, 1 eq) in THF (100 mL) was added NaHCO₃ (17.55 g, 208.96 mmol, 8.13 mL, 3 eq) and Boc₂O (16.72 g, 76.62 mmol, 17.60 mL, 1.1 eq). The mixture was stirred at 20° C. for 4 h. LC-MS analysis indicated that desired mass was detected. TLC (PE:EA=3:1, Rf=0.5) indicated a less polar spot was formed. The reaction mixture was diluted with H₂O (200 mL) and extracted with EA (100 mL×3). The combined organic layers were washed with brine 300 mL (100 mL×3), dried over Na₂SO₄, filtered and concentrated under reduced pressure to give a residue which was further purified by column chromatography (SiO₂, Petroleum ether/Ethyl acetate=1/0 to 3/1) to afford compound tert-Butyl N-(3-chloro-4-hydroxy-phenyl)carbamate (9 g, 36.19 mmol, 51.96% yield, 98% purity) as a black brown solid. ¹H NMR (DMSO-d₆, 400 MHz) δ 9.71 (s, 1H), 9.18 (br s, 1H), 7.47 (br s, 1H), 7.14 (dd, J=2.0, 8.8 Hz, 1H), 6.84 (d, J=8.8 Hz, 1H), 1.45 (s, 9H), LC-MS: [M-t-Bu+H]⁺=188.3.

### Step 2: Preparation of tert-Butyl (3-chloro-4-((2-chloropyridin-4-yl)oxy)phenyl)carbamate

To a solution of tert-Butyl N-(3-chloro-4-hydroxy-phenyl) carbamate (4.4 g, 18.06 mmol, 1 eq) in THF (45 mL) were added t-BuOK (2.03 g, 18.06 mmol, 1 eq) and 2-chloro-4-fluoro-pyridine (2.38 g, 18.06 mmol, 1 eq). The mixture was stirred at 60° C. for 16 h. LC-MS analysis indicated that the desired mass was detected. TLC (PE:EA=3:1, Rf=0.4) showed a new spot was detected. The reaction mixture was diluted with H₂O (50 mL) and extracted with EA 90 (30 mL×3). The combined organic layers were washed with brine (30 mL×3), dried over Na₂SO₄, filtered and concentrated under reduced pressure to give the crude product which was further purified by reversed-phase HPLC (MeCN/H₂O: 100 mL/min; B %: 10-35%, 35 min; 65-100%, 20 min) to afford compound tert-Butyl N-[3-chloro-4-[(2-chloro-4-pyridyl)oxy]phenyl]carbamate (2.4 g, 6.76 mmol, 37.42% yield) as a white solid, LC-MS: [M+H]⁺=355.0.

### Preparation of tert-Butyl (4-((2-chloropyridin-4-yl)oxy)-2-fluorophenyl)carbamate, Int-6

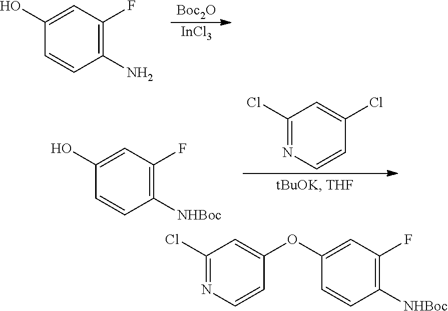

### Step 1: Preparation of tert-Butyl (2-fluoro-4-hydroxyphenyl)carbamate

To a mixture of 4-amino-3-fluoro-phenol (50.0 g, 393.34 mmol, 1 eq) and Boc₂O (85.85 g, 393.34 mmol, 90.36 mL, 1 eq) at 35° C. was slowly added InCl₃ (869.98 mg, 3.93 mmol, 0.01 eq). The reaction mixture was stirred at 35° C. for 3 h. LC-MS analysis indicated full consumption of 4-amino-3-fluoro-phenol and the desired mass was detected. The reaction mixture was concentrated under reduced pressure to give a residue which was further purified by column chromatography (SiO₂, Petroleum ether/Ethyl acetate=1/0 to 3/1) to get tert-Butyl N-(2-fluoro-4-hydroxy-phenyl)carbamate (78.0 g, 343.26 mmol, 87.27% yield) as pink solid. LC-MS: [M+H]⁺=228.0

### Step 2: Preparation of tert-Butyl (4-((2-chloropyridin-4-yl)oxy)-2-fluorophenyl)carbamate

To a solution of tert-Butyl N-(2-fluoro-4-hydroxy-phenyl)carbamate (40.0 g, 176.03 mmol, 1 eq) in NMP (500 mL) was added t-BuOK (23.7 g, 211.24 mmol, 1.2 eq) and 2-chloro-4-fluoro-pyridine (23.15 g, 176.03 mmol, 1 eq). The reaction mixture was stirred at 60° C. for 1 h. LC-MS analysis indicated full consumption of tert-Butyl N-(2-fluoro-4-hydroxy-phenyl)carbamate and the desired mass was detected. The reaction mixture was diluted with ethyl acetate (300 mL), washed with brine (100 mL×5), dried over anhydrous sodium sulfate, filtered and concentrated under reduced pressure to give a residue which was further purified by column chromatography (SiO₂, Petroleum ether/Ethyl acetate=1/0 to 10/1) to get tert-Butyl N-[4-[(2-chloro-4-pyridyl)oxy]-2-fluoro-phenyl]carbamate (50 g, 147.60 mmol, 83.85% yield) as white solid. ¹H NMR (DMSO-d₆, 400 MHz) 9.07 (s, 1H), 8.30 (d, J=6.0 Hz, 1H), 7.71-7.68 (m, 1H), 7.25-7.22 (m, 1H), 7.05-6.99 (m, 2H), 6.98-6.93 (m, 1H), 1.47 (s, 9H), LC-MS: [M+H]⁺=339.0.

### Preparation of 4-((4-bromothiazol-2-yl)oxy)-2-fluoroaniline, Int-7

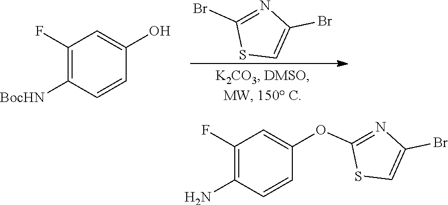 [SMILES: CC(C)(C)OC(=O)Nc1ccc(O)cc1F ; Brc1csc(Br)n1 ; Nc1ccc(Oc2nc(Br)cs2)cc1F]

### Step 1: Preparation of 4-((4-bromothiazol-2-yl)oxy)-2-fluoroaniline

To a solution of tert-Butyl N-(2-fluoro-4-hydroxy-phenyl)carbamate (561.25 mg, 2.47 mmol, 1 eq) in DMSO (30 mL) was added K₂CO₃ (682.74 mg, 4.94 mmol, 2 eq), and 2,4-dibromothiazole (600 mg, 2.47 mmol, 1 eq). The reaction mixture was stirred at 150° C. for 1 h under microwave. LC-MS analysis indicated full consumption of the phenol reactant and mass of the desired product was detected. After cooling to room temperature, the reaction mixture was diluted with water (200 mL) and extracted with EA (100 mL×3). The combined organic layers were washed with brine (100 mL×2), dried over Na₂SO₄, filtered and concentrated under reduced pressure to give a residue which was further purified by column chromatography (SiO₂, Petroleum ether/Ethyl acetate=10/1 to 3/1) to afford 4-(4-bromothiazol-2-yl)oxy-2-fluoro-aniline (4.9 g, 15.25 mmol, 88.22% yield, 90% purity) as a yellow oil. ¹H NMR (DMSO-d₆, 400 MHz) δ 7.25 (s, 1H), 7.22 (dd, J=2.4, 8.0 Hz, 1H), 7.19 (dd, J=2.0, 8.4 Hz, 1H), 7.20 (dd, J=9.6 Hz, 1H), 5.34 (br s, 2H), LC-MS: [M+H]⁺=290.9.

### PREPARATION OF EXEMPLARY COMPOUNDS

### Example 1: Preparation of 1-[4-[4-[3-chloro-4-[(3-methyl-2-pyridyl)methoxy]anilino]-7H-pyrrolo[2,3-d]pyrimidin-5-yl]-1-piperidyl]prop-2-en-1-one, Compound 29

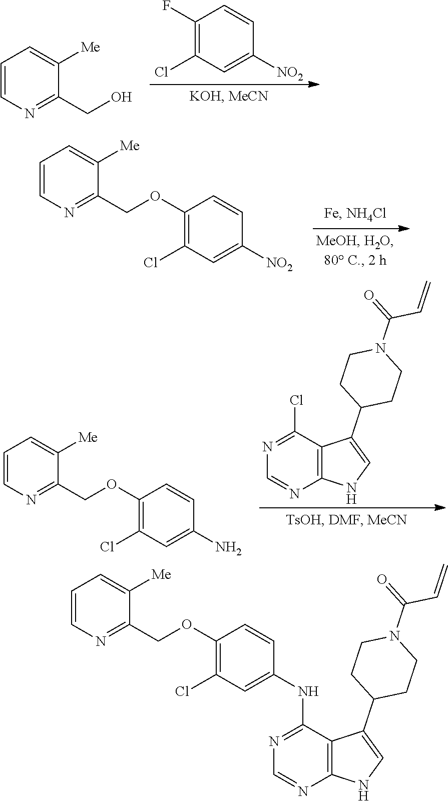

### Step 1: Preparation of 2-[(2-chloro-4-nitro-phenoxy)methyl]-3-methyl-pyridine

To a solution of (3-methyl-2-pyridyl) methanol (2 g, 16.24 mmol, 1 eq) in MeCN (20 mL) was added KOH (1.14 g, 20.30 mmol, 1.25 eq). 2-chloro-1-fluoro-4-nitro-benzene (2.85 g, 16.24 mmol, 1 eq) was added and the reaction mixture was stirred at 70° C. for 10 hours. LC-MS analysis indicated full consumption of (3-methyl-2-pyridyl) methanol and mass of the desired product was detected. The reaction mixture was poured into H₂O (200 mL) and filtered. The filter cake was dried under vacuum to afford 2-[(2-chloro-4-nitro-phenoxy)methyl]-3-methyl-pyridine (4.1 g, 14.56 mmol, 89.68% yield, 99% purity) as a brown solid without further purification ¹H NMR (DMSO-d₆, 400 MHz) δ 8.39 (d, J=4.4 Hz, 1H), 8.31 (br d, J=2.8 Hz, 1H), 8.22-8.19 (m, 1H), 7.68 (br d, J=7.6 Hz, 1H), 7.51 (br d, J=9.2 Hz, 1H), 7.34-7.30 (m, 1H), 5.48 (s, 2H), 2.38 (s, 3H).

### Step 2: Preparation of 3-chloro-4-[(3-methyl-2-pyridyl)methoxy]aniline

To a solution of 2-[(2-chloro-4-nitro-phenoxy)methyl]-3-methyl-pyridine (1 g, 3.55 mmol, 99% purity, 1 eq) and NH₄Cl (1.90 g, 35.52 mmol, 10 eq) in MeOH/H₂O (20 mL/5 mL) was added Fe (1.98 g, 35.52 mmol, 10 eq) and the mixture was stirred at 80° C. for 2 h. LC-MS analysis showed that the 2-[(2-chloro-4-nitro-phenoxy)methyl]-3-methyl-pyridine was consumed completely and the desired mass was detected. The reaction mixture was filtered and the filtrate was concentrated under reduced pressure to give a residue. The residue was diluted with water (50.0 mL) and extracted with ethyl acetate (200 mL×3). The combined organic layers were washed with brine (100 mL), dried over sodium sulfate, filtered and concentrated under reduced pressure to afford 3-chloro-4-[(3-methyl-2-pyridyl)methoxy]aniline (400 mg, 1.29 mmol, 36.22% yield, 80% purity) as a brown solid without purification.

### Step 3: Preparation of 1-[4-[4-[3-chloro-4-[(3-methyl-2-pyridyl)methoxy]anilino]-7H-pyrrolo[2,3-d]pyrimidin-5-yl]-1-piperidyl]prop-2-en-1-one

To a solution of 3-chloro-4-[(3-methyl-2-pyridyl)methoxy]aniline (100.00 mg, 321.66 umol, 80% purity, 1 eq) in DMF (2 mL) and MeCN (2 mL) was added 1-[4-(4-chloro-7H-pyrrolo[2,3-d]pyrimidin-5-yl)-1-piperidyl]prop-2-en-1-one (84.17 mg, 289.50 umol, 0.9 eq) and TsOH (166.17 mg, 964.99 umol, 3 eq). The reaction mixture was stirred at 70° C. for 10 h. LC-MS analysis showed that the 3-chloro-4-[(3-methyl-2-pyridyl)methoxy]aniline was completely consumed and the desired mass was detected. The mixture was poured into saturation of NaHCO₃ (100 mL) and filtered. The filter cake was redissolved in THF (10 mL) and concentrated under vacuum to give a crude product which was further purified by pre-HPLC (column: Phenomenex luna C18 150*25 mm*10 um; mobile phase: [water (FA)-ACN]; B %: 13%-43%) and lyophilization. The 1-[4-[4-[3-chloro-4-[(3-methyl-2-pyridyl) methoxy]anilino]-7H-pyrrolo[2,3-d]pyrimidin-5-yl]-1-piperidyl]prop-2-en-1-one (67 mg, 125.21 umol, 38.93% yield, 94% purity) was obtained as a white solid. ¹H NMR (CDCl₃, 400 MHz) δ 9.90 (br s, 1H), 8.45 (d, J=4.0 Hz, 1H), 8.40 (s, 1H), 7.71 (d, J=2.4 Hz, 1H), 7.54 (d, J=7.6 Hz, 1H), 7.37 (dd, J=2.8, 8.8 Hz, 1H), 7.24-7.17 (m, 2H), 6.89 (s, 1H), 6.68 (s, 1H), 6.63 (dd, J=10.8, 16.8 Hz, 1H), 6.32 (dd, J=2.0, 16.8 Hz, 1H), 5.73 (dd, J=2.0, 10.4 Hz, 1H), 5.33 (s, 2H), 4.94-4.79 (m, 1H), 4.27-4.08 (m, 1H), 3.34-3.14 (m, 1H), 3.04-2.94 (m, 1H), 2.87-2.74 (m, 1H), 2.51 (s, 3H), 2.18 (m, 2H), 1.71-1.63 (m, 2H), LC-MS: [M+H]⁺=503.1.

### Example 2: Preparation of 1-(4-(4-((3-chloro-4-((3-fluoropyridin-2-yl)methoxy)phenyl)amino)-7H-pyrrolo[2,3-d]pyrimidin-5-yl)piperidin-1-yl)prop-2-en-1-one, Compound 37

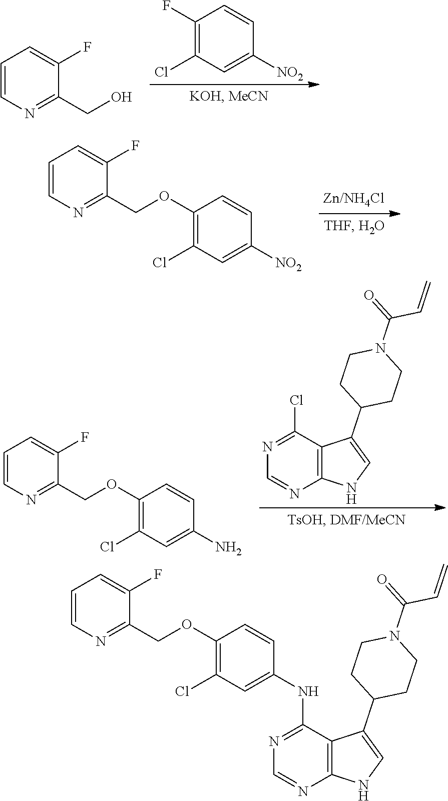

### Step 1: Preparation of 2-[(2-chloro-4-nitro-phenoxy)methyl]-3-fluoro-pyridine

To a solution of (3-fluoro-2-pyridyl) methanol (500 mg, 3.93 mmol, 1 eq) in MeCN (10 mL) was added KOH (331.03 mg, 5.90 mmol, 1.5 eq) and 2-chloro-1-fluoro-4-nitro-benzene (690.49 mg, 3.93 mmol, 1 eq). The reaction mixture was stirred at 25° C. for 12 h. LC-MS analysis indicated complete consumption of the alcohol reactant and desired mass was detected. The reaction mixture was quenched with HCl (0.5 M, 50 mL) at 10° C., and then diluted with EA (10 mL) and extracted with EA (20 mL×2). The combined organic layers were washed with water (20 mL×2), dried over Na₂SO₄, filtered and concentrated under reduced pressure to give compound 2-[(2-chloro-4-nitro-phenoxy)methyl]-3-fluoro-pyridine (1 g, 3.54 mmol, 89.94% yield) as a yellow solid. ¹H NMR (DMSO-d₆, 400 MHz) δ 8.48-8.44 (m, 1H), 8.33 (d, J=2.8 Hz, 1H), 8.24 (dd, J=2.8, 9.2 Hz, 1H), 7.87-7.80 (m, 1H), 7.60-7.52 (m, 2H), 5.55 (d, J=2.0 Hz, 2H), LC-MS: [M+H]⁺=282.9.

### Step 2: Preparation of 3-chloro-4-[(3-fluoro-2-pyridyl)methoxy]aniline

To a solution of 2-[(2-chloro-4-nitro-phenoxy) methyl]-3-fluoro-pyridine (1.0 g, 3.54 mmol, 1 eq) in THF/H₂O (20 mL/4 mL) was added NH₄Cl (3.78 g, 70.76 mmol, 20 eq) and Zn (4.72 g, 72.18 mmol, 20.40 eq) at 0-5° C. The mixture was warmed to 25° C. and stirred for 12 h. LC-MS analysis indicated complete consumption of the nitro reactant and desired mass was detected. TLC (PE:EA=1:1) also indicated full consumption of the reactant. The reaction mixture was filtered and the filter cake was washed with THF (100 mL×3). The resulting organic mixture was diluted with water (100 mL) and extracted with EA (100 mL×3). The combined organic layers were washed with brine (200 mL), dried over Na₂SO₄, filtered and concentrated to give a crude residue which was further purified by prep-TLC (SiO₂, PE:EA=1:1) to afford compound 3-chloro-4-[(3-fluoro-2-pyridyl)methoxy]aniline (480 mg, 1.90 mmol, 53.70% yield) as a yellow solid.

### Step 3: Preparation of 1-[4-[4-[3-chloro-4-[(3-fluoro-2-pyridyl)methoxy]anilino]-7H-pyrrolo[2,3-d]pyrimidin-5-yl]-1-piperidyl]prop-2-en-1-one

To a solution of 3-chloro-4-[(3-fluoro-2-pyridyl)methoxy]aniline (60 mg, 237.46 umol, 1 eq) in DMF/MeCN (0.5 mL/0.5 mL) was added TsOH (122.67 mg, 712.39 umol, 3 eq) and 1-[4-(4-chloro-7H-pyrrolo[2,3-d]pyrimidin-5-yl)-1-piperidyl]prop-2-en-1-one (69.04 mg, 237.46 umol, 1 eq). The mixture was stirred at 70° C. for 12 h. LC-MS analysis showed that the aniline reactant was consumed completely and desired mass was detected. The reaction mixture was quenched with saturated NaHCO₃ aqueous solution (20 mL) at 10° C. A solid was formed and the solid was filtered and concentrated under reduced pressure to give a crude product which was further purified by prep-HPLC (neutral condition; column: Waters Xbridge C18 150*50 mm*10 um; mobile phase: [water(NH₄HCO₃)-ACN]; B %: 32%-62%) to afford compound 1-[4-[4-[3-chloro-4-[(3-fluoro-2-pyridyl)methoxy]anilino]-7H-pyrrolo[2,3-d]pyrimidin-5-yl]-1-piperidyl]prop-2-en-1-one (38 mg, 71.96 umol, 30.30% yield, 96% purity) as a white solid. ¹H NMR (CD₃OD, 400 MHz) δ 8.46-8.40 (m, 1H), 8.15 (s, 1H), 7.73-7.67 (m, 2H), 7.56-7.48 (m, 1H), 7.42 (dd, J=2.8, 8.8 Hz, 1H), 7.25 (d, J=8.8 Hz, 1H), 7.01 (s, 1H), 6.80 (dd, J=10.8, 16.8 Hz, 1H), 6.20 (dd, J=2.0, 16.8 Hz, 1H), 5.74 (dd, J=2.0, 10.8 Hz, 1H), 5.32 (d, J=2.0 Hz, 2H), 4.69 (m, 1H), 4.22 (m, 1H), 3.36 (m, 2H), 2.99-2.87 (m, 1H), 2.27-2.11 (m, 2H), 1.67-1.48 (m, 2H), LC-MS: [M+H]⁺=507.1.

### Example 3: Preparation of 1-[4-[4-[3-chloro-4-(thiazol-4-ylmethoxy)anilino]-7H-pyrrolo[2,3-d]pyrimidin-5-yl]-1-piperidyl]prop-2-en-1-one, Compound 119

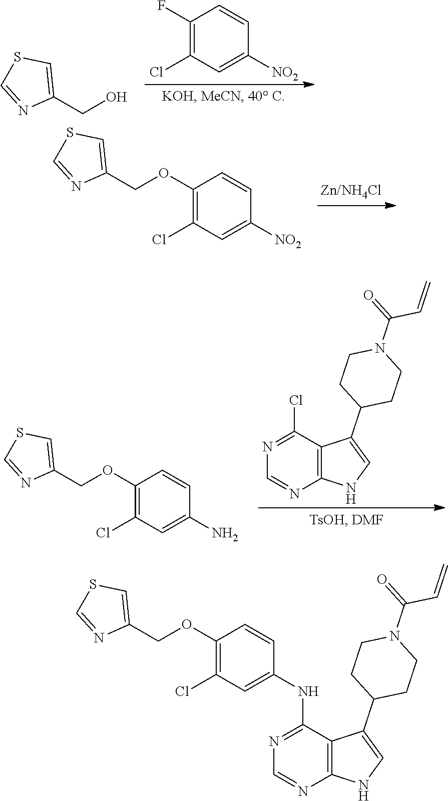

### Step 1: Preparation of 4-[(2-chloro-4-nitro-phenoxy) methyl] thiazole

To a solution of thiazol-4-ylmethanol (3 g, 26.05 mmol, 1 eq) in MeCN (50 mL) was added KOH (1.75 g, 31.26 mmol, 1.2 eq) and the mixture was stirred at 20° C. for 0.5 hour. 2-chloro-1-fluoro-4-nitro-benzene (4.57 g, 26.05 mmol, 1 eq) was added to the above mixture and the reaction mixture was stirred at 40° C. for 2 hours. LC-MS analysis indicated full consumption of the alcohol reactant and mass of the desired product was detected. The mixture was poured into water (500 mL) and light yellow solid precipitated. The resulting suspension was filtered and the filter cake was dried under reduced pressure to give 4-[(2-chloro-4-nitro-phenoxy) methyl]thiazole (4.2 g, 13.65 mmol, 52.41% yield, 88% purity) as a light yellow solid without further purification. ¹H NMR (DMSO-d₆, 400 MHz) δ 9.15 (d, J=2.0 Hz, 1H), 8.32 (d, J=2.8 Hz, 1H), 8.27-8.21 (m, 1H), 7.88 (d, J=1.6 Hz, 1H), 7.57 (d, J=9.2 Hz, 1H), 5.49 (s, 2H), LC-MS: [M+H]⁺=271.0.

### Step 2: Preparation of 3-chloro-4-(thiazol-4-ylmethoxy)aniline

To a solution of 4-[(2-chloro-4-nitro-phenoxy) methyl]thiazole (2 g, 7.39 mmol, 1 eq) in THF/H₂O (10 mL/10 mL) was added NH₄Cl (3.95 g, 73.88 mmol, 10 eq) and Zn (5.41 g, 82.73 mmol, 11.20 eq). The mixture was stirred at 20° C. for 1 hour. LC-MS analysis indicated full consumption of the nitro reactant and mass of the desired product was detected. The reaction mixture was filtered and the filtrate was diluted with ethyl acetate (50 mL), washed with water (50 mL×2), dried over anhydrous sodium sulfate and concentrated under reduced pressure to give 3-chloro-4-(thiazol-4-ylmethoxy) aniline (1.7 g, 7.06 mmol, 95.59% yield) as yellow oil without further purification. ¹H NMR (DMSO-d₆, 400 MHz) δ 9.11 (d, J=2.0 Hz, 1H), 7.71 (d, J=2.0 Hz, 1H), 6.95 (d, J=8.8 Hz, 1H), 6.64 (d, J=2.8 Hz, 1H), 6.51-6.44 (m, 1H), 5.11 (s, 2H), 4.94 (s, 2H), LC-MS: [M+H]⁺=241.0.

### Step 3: Preparation of 1-[4-[4-[3-chloro-4-(thiazol-4-ylmethoxy)anilino]-7H-pyrrolo[2,3-d]pyrimidin-5-yl]-1-piperidyl]prop-2-en-1-one

To a solution of 3-chloro-4-(thiazol-4-ylmethoxy)aniline (993.48 mg, 4.13 mmol, 1 eq) and 1-[4-(4-chloro-7H-pyrrolo[2,3-d]pyrimidin-5-yl)-1-piperidyl]prop-2-en-1-one (1.2 g, 4.13 mmol, 1 eq) in DMF (20 mL) was added TsOH (1.42 g, 8.25 mmol, 2 eq). The mixture was stirred at 70° C. for 12 hours. LC-MS analysis indicated that around 10% of the aniline reactant was remained and mass of the desired compound was detected. The mixture was diluted with ethyl acetate (50 mL), washed with saturated sodium bicarbonate solution (50 ml×3), dried over anhydrous sodium sulfate and concentrated under reduced pressure to give a residue which was triturated with ethyl acetate (30 mL) at 50° C. for 60 min to give 1.1 g off-white solid. The mother liquid was recovered to give 1.2 g brown solid. The off-white solid was purified by prep-HPLC (column: Waters Xbridge C18 150*50 mm*10 um; mobile phase: [water(NH₄HCO₃)-ACN]; B %: 24%-54%, 10 min) to give 600 mg white solid. The brown solid was purified by prep-HPLC (column: Waters Xbridge C18 150*50 mm*10 um; mobile phase: [water(NH4HCO3)-ACN]; B %: 24%-54%, 10 min) to give 350 mg white solid. Compound 1-[4-[4-[3-chloro-4-(thiazol-4-ylmethoxy)anilino]-7H-pyrrolo[2,3-d]pyrimidin-5-yl]-1-piperidyl]prop-2-en-1-one (950 mg, 1.87 mmol, 45.20% yield, 97.2% purity) was obtained as a white solid. ¹H NMR (DMSO-d₆, 400 MHz) δ 11.60 (s, 1H), 9.15 (d, J=2.0 Hz, 1H), 8.21-8.16 (m, 1H), 7.98 (s, 1H), 7.86-7.77 (m, 2H), 7.60-7.52 (m, 1H), 7.30 (d, J=8.8 Hz, 1H), 7.04 (s, 1H), 6.89-6.77 (m, 1H), 6.16-6.06 (m, 1H), 5.70-5.62 (m, 1H), 5.32 (s, 2H), 4.56 (m, 1H), 4.14 (m, 1H), 3.52 (m, 1H), 3.29-3.22 (m, 1H), 2.84 (m, 1H), 2.02 (m, 2H), 1.53-1.35 (m, 2H), LC-MS: [M+H]⁺=495.1.

### Example 4: Preparation of 1-[4-[4-[3-chloro-2-fluoro-4-(2-pyridylmethoxy) anilino]-7H-pyrrolo[2,3-d]pyrimidin-5-yl]-1-piperidyl]prop-2-en-1-one, Compound 106

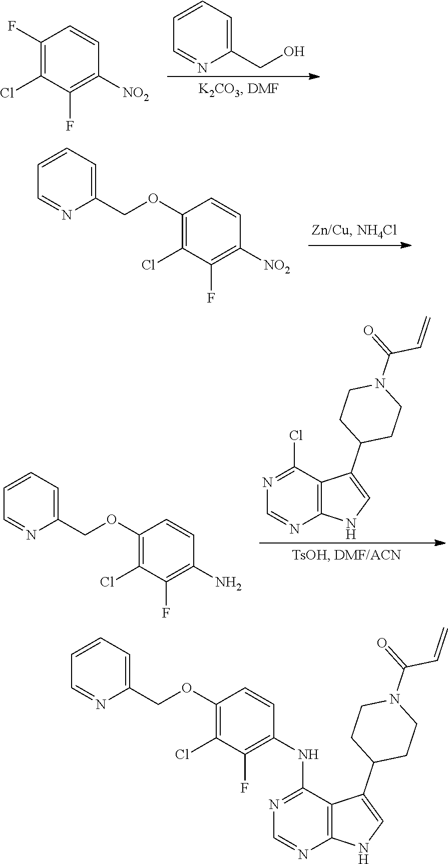

### Step 1: Preparation of 2-[(2-chloro-3-fluoro-4-nitro-phenoxy) methyl] pyridine

To a solution of 2-chloro-1,3-difluoro-4-nitro-benzene (1 g, 5.17 mmol, 1 eq) in DMF (10 mL) was added K₂CO₃ (2.14 g, 15.50 mmol, 3 eq) and 2-pyridylmethanol (563.85 mg, 5.17 mmol, 498.99 uL, 1 eq). The mixture was stirred at 20° C. for 12 h. LC-MS showed that the desired mass was detected and the alcohol reactant was consumed. The reaction mixture was diluted with H₂O (10 mL) and filtered. The filter cake was washed with water (20 mL), collected and was further purified by column chromatography (SiO₂, Petroleum ether/Ethyl acetate=1/0 to 1/1) to afford 2-[(2-chloro-3-fluoro-4-nitro-phenoxy) methyl] pyridine (400 mg, 1.42 mmol, 27.39% yield) as a yellow solid. ¹H NMR (400 MHz, DMSO-d₆) δ 8.60 (d, J=4.0 Hz, 1H), 8.20 (t, J=9.2 Hz, 1H), 7.89 (td, J=1.6, 6.8 Hz, 1H), 7.57 (d, J=7.6 Hz, 1H), 7.36-7.42 (m, 1H), 7.33 (dd, J=1.6, 11.2 Hz, 1H), 5.48 (s, 2H), LC-MS: [M+H]⁺=283.1.

### Step 2: Preparation of 3-chloro-2-fluoro-4-(2-pyridylmethoxy) aniline

To a solution of 2-[(2-chloro-3-fluoro-4-nitro-phenoxy) methyl] pyridine (390 mg, 1.38 mmol, 1 eq) in THF/H₂O (5 mL/1 mL) was added NH₄Cl (738.06 mg, 13.80 mmol, 10 eq). Copper/zinc alloy (1.78 g, 13.80 mmol, 10 eq) was added at 0° C. and the reaction mixture was stirred at 20° C. for 3 h. LC-MS analysis showed that mass of the desired product was detected and the nitro reactant was completely consumed. The reaction mixture was filtered, and the filtrate was washed with water (5 mL×3). The combined organic layers were washed with brine (5 mL×3), dried over Na₂SO₄, filtered and concentrated under reduced pressure to give 3-chloro-2-fluoro-4-(2-pyridylmethoxy)aniline (300 mg, 1.19 mmol, 86.05% yield) as a pink solid without purification. LC-MS: [M+H]⁺=253.4.

### Step 3: Preparation of 1-[4-[4-[3-chloro-2-fluoro-4-(2-pyridylmethoxy) anilino]-7H-pyrrolo[2,3-d]pyrimidin-5-yl]-1-piperidyl]prop-2-en-1-one

To a solution of 3-chloro-2-fluoro-4-(2-pyridylmethoxy)aniline (150 mg, 593.66 umol, 1 eq) in DMF/MeCN (2 mL/2 mL) was added TsOH (306.69 mg, 1.78 mmol, 3 eq) and 1-[4-(4-chloro-7H-pyrrolo[2,3-d]pyrimidin-5-yl)-1-piperidyl]prop-2-en-1-one (172.60 mg, 593.66 umol, 1 eq). The mixture was stirred at 70° C. for 12 h. LC-MS analysis showed that mass of the desired product was detected. The reaction mixture was diluted with saturated NaHCO₃ aqueous solution (10 mL) and extracted with EA (10 mL×3). The combined organic layers were washed with brine (10 mL×3), dried over Na₂SO₄, filtered and concentrated under reduced pressure to give a residue which was purified by prep-TLC (SiO₂, DCM:MeOH=10:1) and was further purified by prep-HPLC (column: Phenomenex C18 75*30 mm*3 um; mobile phase: [water(FA)-ACN]; B %: 22%-52%, 7 min) to afford 1-[4-[4-[3-chloro-2-fluoro-4-(2-pyridylmethoxy)anilino]-7H-pyrrolo[2,3-d]pyrimidin-5-yl]-1-piperidyl]prop-2-en-1-one (92.91 mg, 183.27 umol, 30.87% yield, 100% purity) as an off-white solid. ¹H NMR (400 MHz, DMSO-d₆) δ 11.57 (s, 1H), 8.61 (d, J=4.8 Hz, 1H), 8.07 (s, 1H), 7.96 (s, 1H), 7.89 (t, J=7.6 Hz, 1H), 7.61-7.57 (m, 1H), 7.56-7.49 (m, 1H), 7.41-7.35 (m, 1H), 7.12 (d, J=9.2 Hz, 1H), 7.04 (s, 1H), 6.84 (dd, J=10.4, 16.4 Hz, 1H), 6.10 (dd, J=1.6, 16.8 Hz, 1H), 5.72-5.61 (m, 1H), 5.36 (s, 2H), 4.56 (m, J=10.4 Hz, 1H), 4.22-4.10 (m, 1H), 3.42 (m, 1H), 3.29-3.23 (m, 1H), 2.88-2.77 (m, 1H), 2.11-2.01 (m, 2H), 1.52-1.40 (m, 2H), LC-MS: [M+H]⁺=507.2.

### Example 5: Preparation of 1-[4-[4-[3-chloro-4-(pyridazin-3-ylmethoxy) anilino]-7H-pyrrolo[2,3-d]pyrimidin-5-yl]-1-piperidyl]prop-2-en-1-one, Compound 120

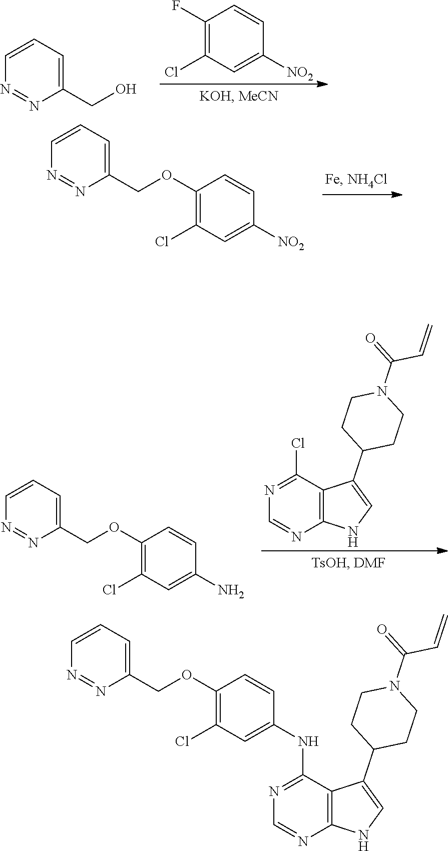

### Step 1: Preparation of 3-[(2-chloro-4-nitro-phenoxy) methyl]pyridazine

The mixture of pyridazin-3-ylmethanol (500.56 mg, 4.55 mmol, 1.05 eq) and KOH (303.63 mg, 5.41 mmol, 1.25 eq) in MeCN (10 mL) was stirred at 20° C. for 0.5 hour, then 2-chloro-1-fluoro-4-nitro-benzene (0.76 g, 4.33 mmol, 1 eq) was added. The reaction mixture was stirred at 40° C. for 12 h. LC-MS analysis showed that 2-chloro-1-fluoro-4-nitro-benzene was fully consumed and the desired mass was detected. The reaction mixture was poured into water (40 mL), then filtered to give 3-[(2-chloro-4-nitro-phenoxy) methyl] pyridazine (1.1 g, 4.14 mmol, 95.64% yield) as a light yellow solid without purification. LC-MS: [M+H]⁺=265.8.

### Step 2: Preparation of 3-chloro-4-(pyridazin-3-ylmethoxy) aniline

To a solution of 3-[(2-chloro-4-nitro-phenoxy) methyl]pyridazine (0.3 g, 1.13 mmol, 1 eq) in EtOH/H₂O (10 mL/2 mL) was added NH₄Cl (604.08 mg, 11.29 mmol, 10 eq). The mixture was cooled to 0° C. and Fe (630.66 mg, 11.29 mmol, 10 eq) was added. The reaction mixture was stirred at 80° C. for 1 hour. LC-MS analysis showed that 3-[(2-chloro-4-nitro-phenoxy) methyl]pyridazine was completely consumed and mass of the desired product was detected. The reaction mixture was filtered and then poured into water (20 mL), extracted by EA (10 mL×3). The organic layers were combined, washed with brine (50 mL), dried over anhydrous Na₂SO₄, filtered and concentrated under reduced pressure to give 3-chloro-4-(pyridazin-3-ylmethoxy)aniline (120 mg, 509.19 umol, 45.09% yield) as a yellow solid. ¹H NMR (DMSO-d₆, 400 MHz) δ 9.20 (dd, J₁=1.2 Hz, J₂=4.4 Hz, 1H), 7.83-7.75 (m, 2H), 6.97 (d, J=7.6 Hz, 1H), 6.65 (d, J=2.8 Hz, 1H), 6.47 (dd, J₁=2.8 Hz, J₂=8.8 Hz, 1H), 6.30 (s, 2H), 4.99 (s, 2H), LC-MS: [M+H]⁺=236.1.

### Step 3: Preparation of 1-[4-[4-[3-chloro-4-(pyridazin-3-ylmethoxy) anilino]-7H-pyrrolo[2,3-d]pyrimidin-5-yl]-1-piperidyl]prop-2-en-1-one

To a solution of 3-chloro-4-(pyridazin-3-ylmethoxy)aniline (80 mg, 339.46 umol, 1 eq) and 1-[4-(4-chloro-7H-pyrrolo[2,3-d]pyrimidin-5-yl)-1-piperidyl]prop-2-en-1-one (88.83 mg, 305.51 umol, 0.9 eq) in CH₃CN/DMF (2 mL/2 mL) was added TsOH (175.37 mg, 1.02 mmol, 3 eq). The reaction mixture was stirred at 75° C. for 12 h. LC-MS analysis showed that 3-chloro-4-(pyridazin-3-ylmethoxy) aniline was fully consumed and mass of the desired product was detected. The reaction mixture was poured into water (20 mL), extracted by EA (10 mL×3). The organic layers were combined and washed with brine (50 mL), dried over anhydrous Na₂SO₄, filtered and concentrated under reduced pressure to give a residue which was further purified by prep-HPLC (neutral condition; column: Waters Xbridge 150*25 mm*5 um; mobile phase: [water(NH₄HCOs)-ACN]; B %: 24%-54%, 8 min) and then lyophilized to afford 1-[4-[4-[3-chloro-4-(pyridazin-3-ylmethoxy)anilino]-7H-pyrrolo[2,3-d]pyrimidin-5-yl]-1-piperidyl]prop-2-en-1-one (11.34 mg, 22.38 umol, 6.59% yield, 96.7% purity) as a white solid. ¹H NMR (DMSO-d₆, 400 MHz) δ 11.62 (s, 1H), 9.24 (dd, J₁=2.0 Hz, J₂=4.8 Hz, 1H), 8.18 (s, 1H), 8.02 (s, 1H), 7.87-7.85 (m, 2H), 7.82 (d, J=4.8 Hz, 1H), 7.60-7.52 (m, 1H), 7.29 (d, J=8.8 Hz, 1H), 7.05 (s, 1H), 6.83 (dd, J₁=10.4 Hz, J₂=16.8 Hz, 1H), 6.10 (dd, J₁=2.8 Hz, J₂=16.8 Hz, 1H), 5.66 (dd, J₁=2.4 Hz, J₂=10.4 Hz, 1H), 5.49 (s, 2H), 4.55 (m, 1H), 4.14 (m, 1H), 3.52 (m, 1H), 3.30-3.26 (m, 1H), 2.83 (m, 1H), 2.04-2.01 (m, 2H), 1.48-1.39 (m, 2H), LC-MS: [M+H]⁺=490.2.

### Example 6: Preparation of 1-(4-(4-((3-chloro-4-(pyridin-2-ylmethoxy)phenyl)amino)-7H-pyrrolo[2,3-d]pyrimidin-5-yl)piperidin-1-yl)prop-2-en-1-one, Compound 156

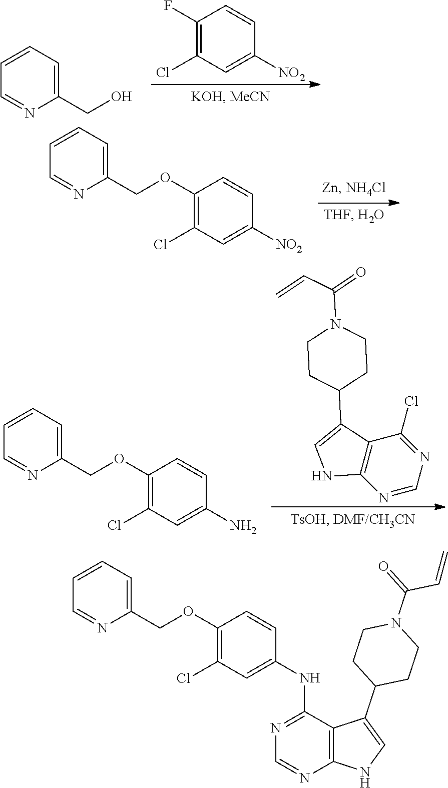

### Step 1: Preparation of 3-chloro-4-(2-pyridylmethoxy)aniline

To a solution of 2-pyridylmethanol (37.86 g, 346.92 mmol, 33.50 mL, 1.05 eq) in MeCN (1 L) was added KOH (23.17 g, 413.00 mmol, 1.25 eq) and the reaction mixture was stirred at 20° C. for 0.5 h. A solution of 2-chloro-1-fluoro-4-nitro-benzene (58 g, 330.40 mmol, 1 eq) in MeCN (100 mL) was added and the reaction mixture was stirred at 35° C. for 11.5 h. LC-MS analysis indicated full consumption of 2-chloro-1-fluoro-4-nitro-benzene and mass of the desired product was detected. The reaction mixture was poured to water (3 L), filtered. The filter cake was concentrated under reduced pressure to afford the crude product which was triturated with MTBE (50 ml) at 20° C. for 30 min. 2-[(2-chloro-4-nitro-phenoxy)methyl]pyridine (80 g, 302.27 mmol, 91.49% yield, 100% purity) was obtained as brown solid. ¹H NMR (DMSO-d₆, 400 MHz) δ 8.60 (d, J=4.8 Hz, 1H), 8.35 (d, J=2.8 Hz, 1H), 8.23 (dd, J₁=2.4 Hz, J₂=9.2 Hz, 1H), 7.89 (d, J=1.6 Hz, 1H), 7.89 (d, J=7.6 Hz, 1H), 7.47 (d, J=9.2 Hz, 1H), 7.38-7.36 (m, 1H), 5.46 (s, 2H), LC-MS: [M+H]⁺=265.0.

### Step 2: Preparation of 3-chloro-4-(2-pyridylmethoxy)aniline

To a solution of 2-[(2-chloro-4-nitro-phenoxy)methyl]pyridine (10 g, 37.78 mmol, 1 eq) and NH₄Cl (40.42 g, 755.67 mmol, 20 eq) in THF/water (100 mL/10 mL) and was added Zn (52.22 g, 798.59 mmol, 21.14 eq) at 0° C. and the reaction mixture was stirred at 20° C. for 12 hours. LC-MS analysis indicated full consumption of 2-[(2-chloro-4-nitro-phenoxy)methyl]pyridine and mass of the desired product was detected. The reaction mixture was filtered, washed with THF (500 mL) and water(1 L). The resulting solution was extracted by EA (500 mL×3). The organic layers were combined and washed with brine (500 mL), dried over Na₂SO₄, concentrated under reduced pressure to afford the crude product which was further purified by trituration with MTBE (500 mL) at 20° C. for 30 min. After filtration and drying under vacuum, 3-chloro-4-(2-pyridylmethoxy)aniline (17 g, 72.44 mmol, 95.86% yield) was obtained as a brown solid. ¹H NMR (DMSO-d₆, 400 MHz) δ 8.55 (d, J=4.0 Hz, 1H), 7.85 (dd, J₁=2.0 Hz, J₂=7.6 Hz, 1H), 7.54 (d, J=7.6 Hz, 1H), 7.33 (d, J=2.0 Hz, 1H), 6.91 (d, J=8.8 Hz, 1H), 6.66 (d, J=2.4 Hz, 1H), 6.46 (dd, J₁=2.4 Hz, J₂=8.4 Hz, 1H), 5.07 (s, 2H), 4.94 (s, 2H), LC-MS: [M+H]⁺=234.9.

### Step 3: Preparation of 1-[4-[4-[3-chloro-4-(2-pyridylmethoxy)anilino]-7H-pyrrolo[2,3-d]pyrimidin-5-yl]-1-piperidyl]prop-2-en-1-one

To a solution of 1-[4-(4-chloro-7H-pyrrolo[2,3-d]pyrimidin-5-yl)-1-piperidyl]prop-2-en-1-one (180 mg, 1 eq) in MeCN/DMF (2 mL/2 mL) was added TsOH (320 mg, 3 eq) and 3-chloro-4-(2-pyridylmethoxy)aniline (145 mg, 1 eq). The mixture was stirred at 70° C. for 12 h. LC-MS analysis indicated that trace of 1-[4-(4-chloro-7H-pyrrolo[2,3-d]pyrimidin-5-yl)-1-piperidyl]prop-2-en-1-one was remained and mass of the desired product was detected. The reaction mixture was poured into NaHCO₃ (saturated, 5 mL), then filtered and give a crude product which was further purified by column chromatography (PE:EA=3:1, 0:1, then EA:DCM:MeOH=5:1:0 to 5:1:1) to afford 1-[4-[4-[3-chloro-4-(2-pyridylmethoxy)anilino]-7H-pyrrolo[2,3-d]pyrimidin-5-yl]-1-piperidyl]prop-2-en-1-one (100 mg, 33.03%) as a white solid. ¹H NMR (DMSO-d₆, 400 MHz) δ 11.60 (s, 1H), 8.53 (s, 1H), 8.60-8.59 (m, 1H), 8.18 (s, 1H), 7.98 (s, 1H), 7.90-7.84 (m, 1H), 7.57 (d, J=7.6 Hz, 1H), 7.53 (dd, J₁=2.4 Hz, J₂=6.4 Hz, 1H), 7.36 (dd, J₁=4.8 Hz, J₂=6.4 Hz, 1H), 7.22 (d, J=8.8 Hz, 1H), 7.04 (d, J=0.8 Hz, 1H), 6.83 (dd, J₁=10.4 Hz, J₂=16.8 Hz, 1H), 6.10 (dd, J₁=2.4 Hz, J₂=24.8 Hz, 1H), 5.66 (dd, J₁=2.4 Hz, J₂=10.4 Hz, 1H), 5.28 (s, 2H), 4.56 (m, 1H), 4.14 (m, 1H), 3.51 (m, 1H), 3.32-3.27 (m, 1H), 2.83 (m, 1H), 2.04-2.02 (m, 2H), 1.45-1.42 (m, 2H), LC-MS: [M+H]⁺=489.3.

### Example 7: Preparation of 1-[4-[4-[3-chloro-4-(pyrazin-2-ylmethoxy)anilino]-7H-pyrrolo[2,3-d]pyrimidin-5-yl]-1-piperidyl]prop-2-en-1-one, Compound 116

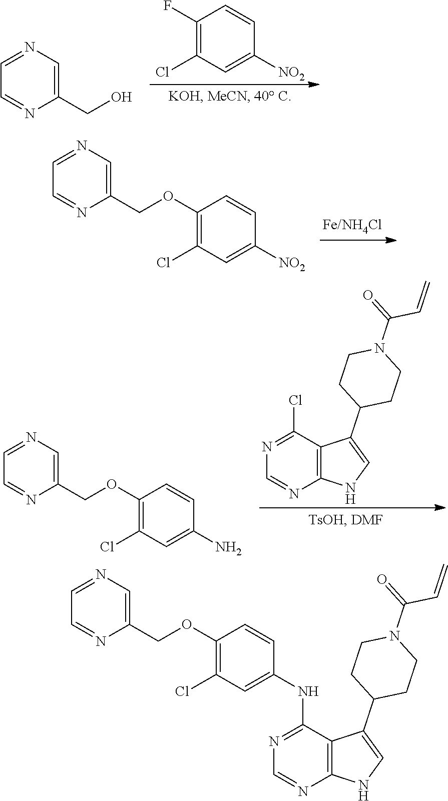

### Step 1: Preparation of 2-[(2-chloro-4-nitro-phenoxy) methyl] pyrazine

To a solution of pyrazin-2-ylmethanol (3.5 g, 31.79 mmol, 1.05 eq) in MeCN (20 mL) was added KOH (2.12 g, 37.84 mmol, 1.25 eq). The reaction mixture was stirred at 20° C. for 0.5 h. 2-chloro-1-fluoro-4-nitro-benzene (5.31 g, 30.27 mmol, 1 eq) was added and the reaction mixture was stirred at 40° C. for 12 h. LC-MS analysis showed that mass of the desired compound was detected. The mixture was added into water (50 mL) and the resulting suspension was filtered and the filter cake was dried under reduced pressure to afford 2-[(2-chloro-4-nitro-phenoxy) methyl] pyrazine (7 g, 26.35 mmol, 87.05% yield) as a yellow solid. LC-MS: [M+H]⁺=266.1.

### Step 2: Preparation of 3-chloro-4-(pyrazin-2-ylmethoxy) aniline

To a solution of 2-[(2-chloro-4-nitro-phenoxy)methyl]pyrazine (7 g, 26.35 mmol, 1 eq) and NH₄Cl (14.10 g, 263.50 mmol, 10 eq) in EtOH/H₂O (25 mL/5 mL) was added Fe (14.72 g, 263.50 mmol, 10 eq). The reaction mixture was stirred at 70° C. for 1 h. LC-MS analysis showed that mass of the desired compound was detected. The mixture was filtered and the filtrate was diluted with H₂O (100 mL), extracted with EA (50 mL×2). The combined organic layers were washed with brine (50 mL), dried over Na₂SO₄, filtered and the filtrate was concentrated to give 3-chloro-4-(pyrazin-2-ylmethoxy) aniline (6.2 g, 26.31 mmol, 99.84% yield) as a yellow solid. ¹H NMR (DMSO-d₆, 400 MHz) δ 8.86 (s, 1H), 8.73-8.66 (m, 2H), 7.02 (d, J=8.8 Hz, 1H), 6.72 (d, J=2.4 Hz, 1H), 6.59-6.50 (m, 1H), 5.22 (s, 2H), 5.05 (s, 2H), LC-MS: [M+H]⁺=236.1.

### Step 3: Preparation of 1-[4-[4-[3-chloro-4-(pyrazin-2-ylmethoxy)anilino]-7H-pyrrolo[2,3-d]pyrimidin-5-yl]-1-piperidyl]prop-2-en-1-one

To a solution of 1-[4-(4-chloro-7H-pyrrolo[2,3-d]pyrimidin-5-yl)-1-piperidyl]prop-2-en-1-one (1.5 g, 5.16 mmol, 1 eq) and 3-chloro-4-(pyrazin-2-ylmethoxy)aniline (1.22 g, 5.16 mmol, 1 eq) in MeCN/DMF (10 mL/10 mL) was added TsOH (2.67 g, 15.48 mmol, 3 eq). The mixture was stirred at 75° C. for 12 h. LC-MS analysis showed that mass of the desired product was detected. The reaction mixture was neutralized with NaHCO₃ to reach pH=7. The resulting mixture was diluted with water (50 mL) and extracted with EA (25 mL×3). The combined organic layers were washed with brine (25 mL×2), dried over Na₂SO₄, filtered and concentrated under reduced pressure to give a residue which was purified by prep-HPLC (neutral condition, column: Waters Xbridge 150*25 mm*5 um; mobile phase: [water(NH₄HCO₃)-ACN]; B %: 28%-58%, 10 min). The residue was further purified by prep-HPLC (column: Waters Xbridge C18 150*50 mm*10 um; mobile phase: [water(NH₄HCO₃)-ACN]; B %: 23%-53%, 10 min) to afford 1-[4-[4-[3-chloro-4-(pyrazin-2-ylmethoxy)anilino]-7H-pyrrolo[2,3-d]pyrimidin-5-yl]-1-piperidyl]prop-2-en-1-one (535 mg, 1.07 mmol, 20.74% yield, 98% purity) as a white solid. ¹H NMR (DMSO-d₆, 400 MHz) δ 11.65-11.55 (m, 1H), 8.86 (s, 1H), 8.73-8.61 (m, 2H), 8.19 (s, 1H), 8.00 (s, 1H), 7.85 (br d, J=2.4 Hz, 1H), 7.63-7.48 (m, 1H), 7.29 (d, J=8.8 Hz, 1H), 7.05 (s, 1H), 6.94-6.74 (m, 1H), 6.12 (d, J=2.0 Hz, 1H), 5.75-5.58 (m, 1H), 5.37 (s, 2H), 4.56 (m, 1H), 4.26-4.05 (m, 1H), 3.63-3.44 (m, 1H), 3.30-3.22 (m, 1H), 2.84 (s, 1H), 2.09-1.98 (m, 2H), 1.44 (m, 2H), LC-MS: [M+H]⁺=490.2.

### Example 8: Preparation of N-[4-[2-chloro-4-[[5-(1-prop-2-enoyl-4-piperidyl)-7H-pyrrolo[2,3-d]pyrimidin-4-yl]amino]phenoxy]-2-pyridyl]cyclopentanecarboxamide, Compound 38

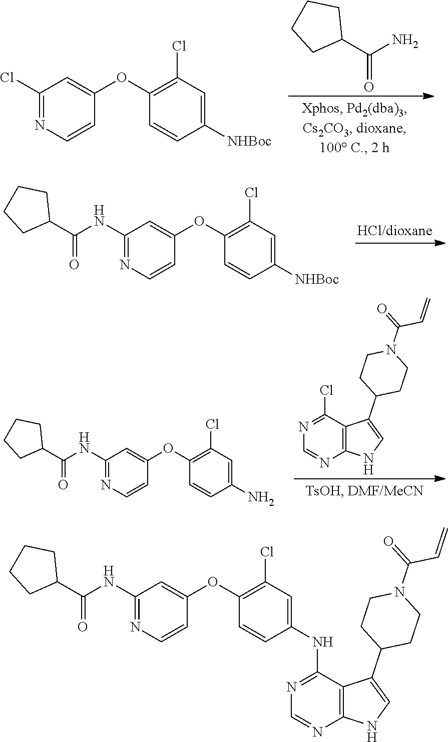 [SMILES: CC(C)(C)OC(=O)Nc1ccc(Oc2ccnc(Cl)c2)c(Cl)c1 ; NC(=O)C1CCCC1 ; CC(C)(C)OC(=O)Nc1ccc(Oc2ccnc(NC(=O)C3CCCC3)c2)c(Cl)c1 ; Nc1ccc(Oc2ccnc(NC(=O)C3CCCC3)c2)c(Cl)c1 ; C=CC(=O)N1CCC(c2c[nH]c3ncnc(Cl)c23)CC1 ; C=CC(=O)N1CCC(c2c[nH]c3ncnc(Nc4ccc(Oc5ccnc(NC(=O)C6CCCC6)c5)c(Cl)c4)c23)CC1]

### Step 1: Preparation of tert-Butyl N-[3-chloro-4-[[2-(cyclopentanecarbonylamino)-4-pyridyl]oxy]phenyl]carbamate

To a solution of tert-Butyl N-[3-chloro-4-[(2-chloro-4-pyridyl)oxy]phenyl]carbamate (350 mg, 985.32 umol, 1 eq) and cyclopentanecarboxamide (278.74 mg, 2.46 mmol, 2.5 eq) in dioxane (15 mL) was added Cs₂CO₃ (642.07 mg, 1.97 mmol, 2 eq), XPhos (93.94 mg, 197.06 umol, 0.2 eq) and Pd₂(dba)₃ (360.91 mg, 394.13 umol, 0.4 eq) at 20° C. under N₂. The reaction mixture was stirred for 2 h at 100° C. under N₂. LC-MS analysis indicated full consumption of tert-Butyl N-[3-chloro-4-[(2-chloro-4-pyridyl)oxy]phenyl]carbamate and the desired mass was detected. The mixture was concentrated under vacuum to get a residue which was purified by column (PE:EA=1:0 to 3:1) to afford tert-Butyl N-[3-chloro-4-[[2-(cyclopentanecarbonylamino)-4-pyridyl]oxy]phenyl]carbamate (300 mg, 541.78 umol, 54.99% yield, 78% purity) as a yellow solid. LC-MS: [M+H]⁺=432.0.

### Step 2: Preparation of N-[4-(4-amino-2-chloro-phenoxy)-2-pyridyl]cyclopentanecarboxamide

To a solution of tert-Butyl N-[3-chloro-4-[[2-(cyclopentanecarbonylamino)-4-pyridyl]oxy]phenyl]carbamate (100 mg, 231.53 umol, 1 eq) in HCl/dioxane (3 mL) was stirred at 20° C. for 1 h. LC-MS analysis indicated full consumption of tert-Butyl N-[3-chloro-4-[[2-(cyclopentanecarbonylamino)-4-pyridyl]oxy]phenyl]carbamate and the desired mass was detected. The mixture was concentrated under vacuum to afford N-[4-(4-amino-2-chloro-phenoxy)-2-pyridyl] cyclopentanecarboxamide (80 mg, 178.14 umol, 76.94% yield, 82% purity, HCl salt) as a brown solid without further purification. LC-MS: [M+H]⁺=332.0.

### Step 3: Preparation of N-[4-[2-chloro-4-[[5-(1-prop-2-enoyl-4-piperidyl)-7H-pyrrolo[2,3-d]pyrimidin-4-yl]amino]phenoxy]-2-pyridyl]cyclopentanecarboxamide

To a solution of N-[4-(4-amino-2-chloro-phenoxy)-2-pyridyl]cyclopentanecarboxamide (60 mg, 133.60 umol, 82% purity, 1 eq, HCl salt) in DMF/MeCN (2 mL/2 mL) was added 1-[4-(4-chloro-7H-pyrrolo[2,3-d]pyrimidin-5-yl)-1-piperidyl]prop-2-en-1-one (34.96 mg, 120.24 umol, 0.9 eq) and TsOH (69.02 mg, 400.81 umol, 3 eq). The reaction mixture was stirred at 70° C. for 10 h. LC-MS analysis indicated full consumption of N-[4-(4-amino-2-chloro-phenoxy)-2-pyridyl]cyclopentanecarboxamide and the desired mass was detected. The mixture was poured into saturated NaHCO₃ (aqueous, 100 mL) and filtered. The filter cake was concentrated under reduced pressure. The crude product was purified by pre-HPLC[column: Waters Xbridge C18 150*50 mm*10 um; mobile phase: [water(NH₄HCOs)-ACN]; B %: 34%-64%, 10 min] and lyophilized to afford N-[4-[2-chloro-4-[[5-(1-prop-2-enoyl-4-piperidyl)-7H-pyrrolo[2,3-d]pyrimidin-4-yl]amino]phenoxy]-2-pyridyl]cyclopentanecarboxamide (4.06 mg, 6.65 umol, 4.98% yield, 96% purity) as a white solid. ¹H NMR (400 MHz, DMSO-d₆) δ 11.71 (s, 1H), 10.51 (s, 1H), 8.31-8.28 (m, 1H), 8.24 (s, 1H), 8.20 (d, J=6.0 Hz, 1H), 8.13-8.11 (m, 1H), 7.81-7.77 (m, 1H), 7.67 (d, J=2.4 Hz, 1H), 7.36 (d, J=8.8 Hz, 1H), 7.11 (d, J=2.0 Hz, 1H), 6.84 (dd, J=10.4, 16.4 Hz, 1H), 6.69 (dd, J=2.4, 5.6 Hz, 1H), 6.10 (dd, J=2.0, 16.4 Hz, 1H), 5.68-5.64 (m, 1H), 4.58 (m, 1H), 4.17 (m, 1H), 3.62-3.55 (m, 1H), 3.27-3.24 (m, 1H), 2.92-2.83 (m, 2H), 2.07-2.01 (m, 2H), 1.83-1.77 (m, 2H), 1.65-1.58 (m, 4H), 1.50-1.40 (m, 4H), LC-MS: [M+H]⁺=586.2.

### Example 9: Preparation of 1-[4-[4-[3-chloro-4-[[2-[3-(trifluoromethyl)pyrrolidin-1-yl]-4-pyridyl]oxy]anilino]-7H-pyrrolo[2,3-d]pyrimidin-5-yl]-1-piperidyl]prop-2-en-1-one, Compound 43

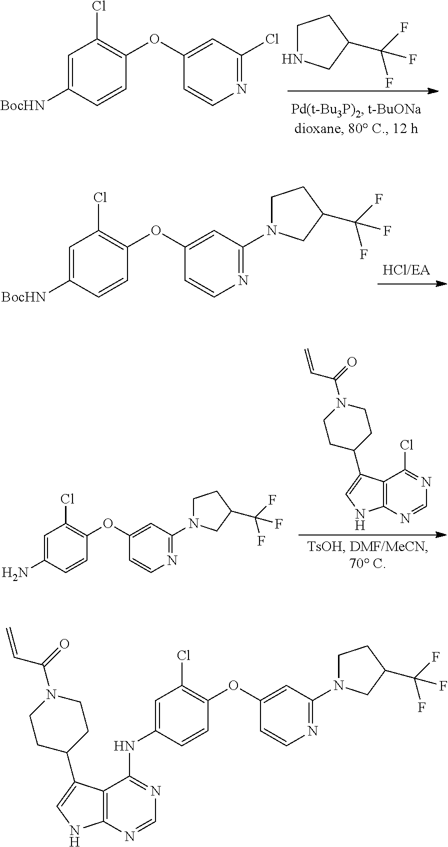

### Step 1: Preparation of tert-Butyl N-[3-chloro-4-[[2-[3-(trifluoromethyl) pyrrolidin-1-yl]-4-pyridyl]oxy]phenyl]carbamate

A mixture of tert-Butyl N-[3-chloro-4-[(2-chloro-4-pyridyl)oxy]phenyl]carbamate (500 mg, 1.41 mmol, 1 eq), 3-(trifluoromethyl)pyrrolidine (247.15 mg, 1.41 mmol, 1 eq, HCl salt), XPhos (402.61 mg, 844.56 umol, 0.6 eq), Cs₂CO₃ (1.38 g, 4.22 mmol, 3 eq) in dioxane (10 mL) was degassed and purged with N₂ for 3 times, and then Pd₂(dba)₃ (257.79 mg, 281.52 umol, 0.2 eq) was added. The reaction mixture was stirred at 70° C. for 4 h under N₂ atmosphere. LC-MS analysis showed that desired mass was detected. The reaction mixture was diluted with H₂O (10 mL) and extracted with EA (10 mL×3). The combined organic layers were washed with brine (10 mL×3), dried over Na₂SO₄, filtered and concentrated under reduced pressure to give a residue which was further purified by column chromatography (SiO₂, Petroleum ether/Ethyl acetate-2/1) to afford compound tert-Butyl N-[3-chloro-4-[[2-[3-(trifluoromethyl) pyrrolidin-1-yl]-4-pyridyl]oxy]phenyl]carbamate (280 mg, 611.52 umol, 43.44% yield) as a yellow oil. LC-MS: [M+H]⁺=458.1.

### Step 2: Preparation of 3-chloro-4-[[2-[3-(trifluoromethyl)pyrrolidin-1-yl]-4-pyridyl]oxy]aniline

To a solution of tert-Butyl N-[3-chloro-4-[[2-[3-(trifluoromethyl)pyrrolidin-1-yl]-4-pyridyl]oxy]phenyl]carbamate (280 mg, 611.52 umol, 1 eq) in EA (1 mL) was added a solution of HCl in EtOAc (4 M, 5 mL, 32.71 eq) at 0° C. The mixture was stirred at 20° C. for 2 h. LC-MS analysis showed that desired mass was detected. The reaction mixture was under reduced pressure to give compound 3-chloro-4-[[2-[3-(trifluoromethyl)pyrrolidin-1-yl]-4-pyridyl]oxy]aniline (200 mg, 507.33 umol, 82.96% yield, HCl salt) as a yellow solid. LC-MS: [M+H]⁺=358.0

### Step 3: Preparation of 1-[4-[4-[3-chloro-4-[[2-[3-(trifluoromethyl)pyrrolidin-1-yl]-4-pyridyl]oxy]anilino]-7H-pyrrolo[2,3-d]pyrimidin-5-yl]-1-piperidyl]prop-2-en-1-one

To a solution of 3-chloro-4-[[2-[3-(trifluoromethyl)pyrrolidin-1-yl]-4-pyridyl]oxy]aniline (80 mg, 202.93 umol, 1 eq, HCl salt) in DMF/MeCN (0.2 mL/0.2 mL) were added TsOH (104.84 mg, 608.80 umol, 3 eq) and 1-[4-(4-chloro-7H-pyrrolo[2,3-d]pyrimidin-5-yl)-1-piperidyl]prop-2-en-1-one (59.00 mg, 202.93 umol, 1 eq). The reaction mixture was stirred at 70° C. for 4 hr. LC-MS analysis showed that desired mass was detected. The reaction mixture was diluted with NaHCO₃ (saturated aqueous solution, 3 mL) and extracted with EA (5 mL×3). The combined organic layers were washed with brine (5 mL×3), dried over Na₂SO₄, filtered and concentrated under reduced pressure to give a residue which was purified by prep-TLC (SiO₂, DCM:MeOH=10:1) and further purified by prep-HPLC (column: Phenomenex C18 75*30 mm*3 um; mobile phase: [water (FA)-ACN]; B %: 12%-42%, 7 min). The residue was last purified by prep-HPLC (column: Waters Xbridge C18 150*50 mm*10 um; mobile phase: [water (NH₄HCOs)-ACN]; B %: 38%-68%, 10 min) to afford 1-[4-[4-[3-chloro-4-[[2-[3-(trifluoromethyl)pyrrolidin-1-yl]-4-pyridyl]oxy]anilino]-7H-pyrrolo[2,3-d]pyrimidin-5-yl]-1-piperidyl]prop-2-en-1-one (18.47 mg, 29.94 umol, 14.75% yield, 99.2% purity) as a white solid. ¹H NMR (DMSO-d₆, 400 MHz) δ 11.70 (br, 1H), 8.27 (s, 1H) 8.20 (s, 1H), 8.11-8.07 (m, 1H), 7.99 (d, J=5.8 Hz, 1H), 7.79-7.72 (m, 1H), 7.30 (d, J=8.8 Hz, 1H), 7.11 (s, 1H) 6.88-6.77 (m, 1H), 6.13-6.07 (m, 2H), 6.01 (d, J=2.0 Hz, 1H), 5.67 (dd, J=2.4, 10.4 Hz, 1H), 4.62-4.52 (m, 1H), 4.22-4.11 (m, 1H), 3.67 (dd, J=8.4, 10.8 Hz, 1H), 3.62-3.54 (m, 1H), 3.46 (dd, J=6.4, 10.8 Hz, 2H), 3.41-3.34 (m, 2H), 3.26 (br, 1H), 2.91-2.79 (m, 1H), 2.26 (dd, J=5.6, 13.2 Hz, 1H), 2.10-2.02 (m, 3H), 1.52-1.41 (m, 2H), LC-MS: [M+H]⁺=612.2.

### Example 10: Preparation of 1-[4-[4-[4-[[2-(3,3-difluoropyrrolidin-1-yl)-4-pyridyl]oxy]-2-fluoro-anilino]-1H-pyrazolo[3,4-d]pyrimidin-3-yl]-1-piperidyl]prop-2-en-1-one, Compound 145

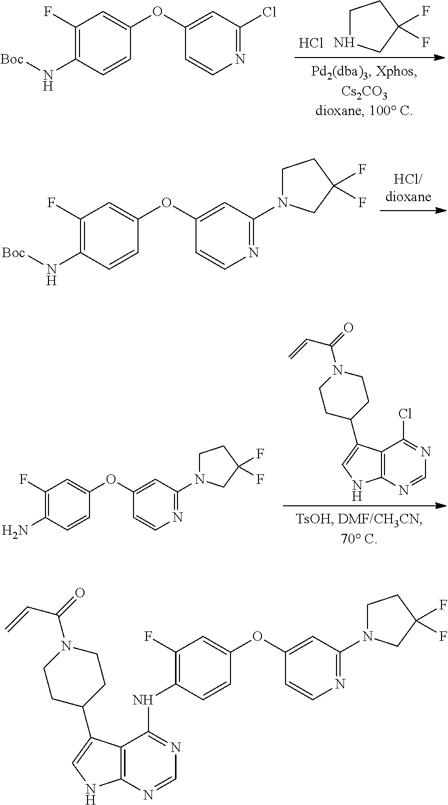

### Step 1: Preparation of tert-Butyl N-[4-[[2-(3,3-difluoropyrrolidin-1-yl)-4-pyridyl]oxy]-2-fluoro-phenyl]carbamate

To a solution of tert-Butyl N-[4-[(2-chloro-4-pyridyl)oxy]-2-fluoro-phenyl]carbamate (500 mg, 1.48 mmol, 1 eq), 3,3-difluoropyrrolidine; hydrochloride (797.13 mg, 4.43 mmol, 3 eq, HCl) in dioxane (10 mL) was added Pd₂(dba)₃ (135.16 mg, 147.60 umol, 0.1 eq), XPhos (140.72 mg, 295.19 umol, 0.2 eq) and Cs₂CO₃ (1.20 g, 3.69 mmol, 2.5 eq). The reaction mixture was purged with N₂ for three times and stirred at 100° C. for 4 h. LC-MS analysis showed that the tert-Butyl N-[4-[(2-chloro-4-pyridyl)oxy]-2-fluoro-phenyl]carbamate was fully consumed and the desired mass was detected. The mixture was concentrated under vacuum to give a crude residue which was further purified by column (SiO₂, PE:EA=100:1-5:1, TLC, PE:EA=5:1, Rf=0.4) to afford tert-Butyl N-[4-[[2-(3,3-difluoropyrrolidin-1-yl)-4-pyridyl]oxy]-2-fluoro-phenyl]carbamate (300 mg, 710.79 umol, 48.16% yield, 97% purity) as a brown solid. LC-MS: [M+H]⁺=410.1.

### Step 2: Preparation of 4-[[2-(3,3-difluoropyrrolidin-1-yl)-4-pyridyl]oxy]-2-fluoro-aniline

A mixture of tert-Butyl N-[4-[[2-(3,3-difluoropyrrolidin-1-yl)-4-pyridyl]oxy]-2-fluoro-phenyl]carbamate (200 mg, 473.86 umol, 97% purity, 1 eq) in HCl/dioxane (5 mL) was stirred at 25° C. for 16 h. LC-MS analysis showed that the tert-Butyl N-[4-[[2-(3,3-difluoropyrrolidin-1-yl)-4-pyridyl]oxy]-2-fluoro-phenyl]carbamate was completely consumed and the desired mass was detected. The mixture was concentrated under vacuum to afford 4-[[2-(3,3-difluoropyrrolidin-1-yl)-4-pyridyl]oxy]-2-fluoro-aniline (160 mg, 462.77 umol, 97.66% yield, HCl salt) as a brown solid without further purification. LC-MS: [M+H]⁺=310.1

### Step 3: Preparation of 1-[4-[4-[4-[[2-(3,3-difluoropyrrolidin-1-yl)-4-pyridyl]oxy]-2-fluoro-anilino]-1H-pyrazolo[3,4-d]pyrimidin-3-yl]-1-piperidyl]prop-2-en-1-one

To a solution of 1-[4-(4-chloro-1H-pyrazolo[3,4-d]pyrimidin-3-yl)-1-piperidyl]prop-2-en-1-one (50 mg, 171.39 umol, 1 eq) in DMF/MeCN (0.5 mL/0.5 mL) was added 4-[[2-(3,3-difluoropyrrolidin-1-yl)-4-pyridyl]oxy]-2-fluoro-aniline (65.18 mg, 188.53 umol, 1.1 eq, HCl salt) and TsOH (88.54 mg, 514.16 umol, 3 eq). The reaction mixture was stirred at 70° C. for 16 h. LC-MS analysis showed that the 1-[4-(4-chloro-1H-pyrazolo [3,4-d]pyrimidin-3-yl)-1-piperidyl]prop-2-en-1-one was fully consumed and the desired mass was detected. The mixture was poured into saturated NaHCO₃ (50 mL) and filtered. The filter cake was dissolved with THF (10 mL) and concentrated under vacuum. The residue was purified by pre-HPLC [column: Phenomenex luna C18 150*25 mm*10 um; mobile phase: [water (FA)-ACN]; B %: 14%-44%, 2 min] and further purified by pre-HPLC (column: Waters Xbridge 150*25 mm*5 um; mobile phase: [water (NH₄HCOs)-ACN]; B %: 37%-67%, 9 min) and lyophilized to afford 1-[4-[4-[4-[[2-(3,3-difluoropyrrolidin-1-yl)-4-pyridyl]oxy]-2-fluoro-anilino]-1H-pyrazolo[3,4-d]pyrimidin-3-yl]-1-piperidyl]prop-2-en-1-one (7.01 mg, 12.32 umol, 7.19% yield, 99.2% purity) as a white solid. ¹H NMR (DMSO-d₆, 400 MHz) δ 11.62 (br, 1H), 8.20-8.13 (m, 1H), 8.05 (d, J=6.0 Hz, 1H), 7.95-7.87 (m, 2H), 7.20 (dd, J=2.4, 11.2 Hz, 1H), 7.01-7.064 (m, 2H), 6.85 (dd, J=10.4, 16.4 Hz, 1H), 6.27 (dd, J=1.6, 6.4 Hz, 1H), 6.16 (d, J=1.6 Hz, 1H), 6.11 (dd, J=3.2, 18.8 Hz, 1H), 5.67 (dd, J=2.4, 10.4 Hz, 1H), 4.58 (m, 1H), 4.17 (m, 1H), 3.82 (t, J=13.6 Hz, 2H), 3.58 (t, J=7.2 Hz, 2H), 3.43 (m, 1H), 3.26-3.23 (m, 1H), 2.85-2.76 (m, 1H), 2.57-2.54 (m, 2H), 2.08 (m, 2H), 1.52-1.43 (m, 2H), LC-MS: [M+H]⁺=564.2.

### Example 11: Preparation of 1-[4-[4-[2-fluoro-4-[[2-(3-hydroxy-3-isopropyl-azetidin-1-yl)-4-pyridyl]oxy]anilino]-7H-pyrrolo[2,3-d]pyrimidin-5-yl]-1-piperidyl]prop-2-en-1-one, Compound 130

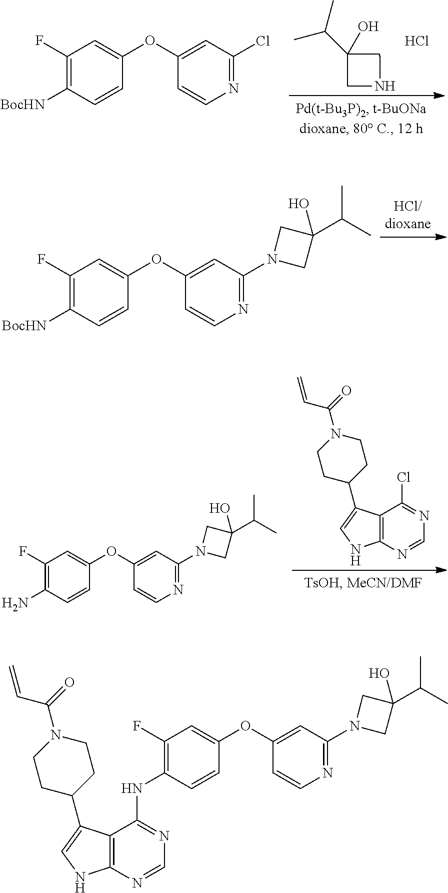

### Step 1: Preparation of tert-Butyl N-[2-fluoro-4-[[2-(3-hydroxy-3-isopropyl-azetidin-1-yl)-4-pyridyl]oxy]phenyl]carbamate

A mixture of tert-Butyl N-[4-[(2-chloro-4-pyridyl)oxy]-2-fluoro-phenyl]carbamate (500 mg, 1.48 mmol, 1 eq), 3-isopropylazetidin-3-ol; hydrochloride (277.62 mg, 1.48 mmol, 1 eq, HCl salt), palladium; tritert-Butylphosphane (150.86 mg, 295.19 umol, 0.2 eq), t-BuONa (425.54 mg, 4.43 mmol, 3 eq) in dioxane (20 mL) was degassed and purged with N₂ for 3 times. The reaction mixture was stirred at 90° C. for 2 hours under N₂ atmosphere. The reaction mixture was filtered and the filtrate was concentrated under reduced pressure to give a residue which was purified by column chromatography (SiO₂, Petroleum ether/Ethyl acetate=1/0 to 1/1) to afford tert-Butyl N-[2-fluoro-4-[[2-(3-hydroxy-3-isopropyl-azetidin-1-yl)-4-pyridyl]oxy]phenyl]carbamate (290 mg, 682.15 umol, 46.22% yield, 98.2% purity) as a white solid. ¹H NMR (CDCl₃, 400 MHz) δ 8.09 (t, J=7.6 Hz, 1H), 8.02 (d, J=5.6 Hz, 1H), 6.91-6.81 (m, 2H), 6.65 (br s, 1H), 6.30-6.14 (m, 1H), 5.76 (d, J=1.6 Hz, 1H), 3.98 (d, J=8.4 Hz, 2H), 3.78 (d, J=8.4 Hz, 2H), 2.03-1.94 (m, 2H), 1.54 (s, 9H), 1.24 (d, J=14.0 Hz, 1H), 0.98 (d, J=6.8 Hz, 6H), LC-MS: [M+H]⁺=418.3.

### Step 2: Preparation of 1-[4-(4-amino-3-fluoro-phenoxy)-2-pyridyl]-3-Isopropyl-azetidin-3-ol

To a solution of tert-Butyl N-[2-fluoro-4-[[2-(3-hydroxy-3-isopropyl-azetidin-1-yl)-4-pyridyl]oxy]phenyl]carbamate (270 mg, 646.75 umol, 1 eq) in dioxane (3 mL) was added HCl/dioxane (4 M, 5 mL, 30.92 eq). The mixture was stirred at 20° C. for 2 h. The mixture was concentrated under reduced pressure to give 1-[4-(4-amino-3-fluoro-phenoxy)-2-pyridyl]-3-isopropyl-azetidin-3-ol (210 mg, 593.53 umol, 91.77% yield, HCl salt) as a white solid. LC-MS: [M+H]⁺=318.2.

### Step 3: Preparation of 1-[4-[4-[2-fluoro-4-[[2-(3-hydroxy-3-isopropyl-azetidin-1-yl)-4-pyridyl]oxy]anilino]-7H-pyrrolo[2,3-d]pyrimidin-5-yl]-1-piperidyl]prop-2-en-1-one

To a solution of 1-[4-(4-amino-3-fluoro-phenoxy)-2-pyridyl]-3-isopropyl-azetidin-3-ol (100 mg, 282.63 umol, 1 eq, HCl salt) and 1-[4-(4-chloro-7H-pyrrolo[2,3-d]pyrimidin-5-yl)-1-piperidyl]prop-2-en-1-one (82.17 mg, 282.63 umol, 1 eq) in DMF/MeCN (2 mL/2 mL) was added TsOH (97.34 mg, 565.26 umol, 2 eq). The mixture was stirred at 70° C. for 48 h. The reaction mixture was diluted with ethyl acetate (30 mL), washed with saturated NaHCO₃ solution (30 mL×2), dried over anhydrous sodium sulfate, filtered and concentrated under reduced pressure to give a residue which was purified by prep-HPLC (column: Waters Xbridge 150*25 mm*5 um; mobile phase: [water (NH₄HCO₃)-ACN]; B %: 29%-59%, 9 min) and was further purified by prep-HPLC (column: Phenomenex luna C18 150*25 mm*10 um; mobile phase: [water(FA)-ACN]; B %: 10%-40%, 2 min). 1-[4-[4-[2-fluoro-4-[[2-(3-hydroxy-3-isopropyl-azetidin-1-yl)-4-pyridyl]oxy]anilino]-7H-pyrrolo[2,3-d]pyrimidin-5-yl]-1-piperidyl]prop-2-en-1-one (8 mg, 13.83 umol, 4.89% yield, 98.8% purity) was obtained as a white solid. ¹H NMR (CD₃OD, 400 MHz) δ 8.19 (s, 1H), 8.10 (t, J=8.8 Hz, 1H), 7.92 (d, J=6.0 Hz, 1H), 7.12-6.98 (m, 3H), 6.88-6.77 (m, 1H), 6.39-6.33 (m, 1H), 6.25-6.16 (m, 1H), 5.95 (d, J=2.0 Hz, 1H), 5.78-5.72 (m, 1H), 4.73 (m, 1H), 4.27 (m, 1H), 3.97 (m, 2H), 3.74 (m, 2H), 3.38 (t, J=12.0 Hz, 2H), 2.96 (t, J=12.8 Hz, 1H), 2.24 (t, J=11.2 Hz, 2H), 2.00-1.88 (m, 1H), 1.73-1.59 (m, 2H), 0.97 (d, J=6.8 Hz, 6H), LC-MS: [M+H]⁺=572.6.

### Example 12: Preparation of 1-[4-[4-[4-[[2-(cycloButylamino)-4-pyridyl]oxy]-2-fluoro-anilino]-7H-pyrrolo[2,3-d]pyrimidin-5-yl]-1-piperidyl]prop-2-en-1-one, Compound 109

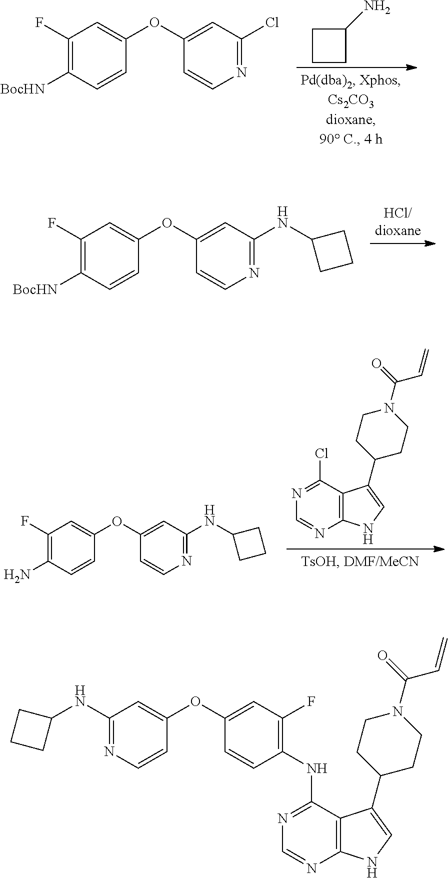

### Step 1: Preparation of tert-Butyl N-[4-[[2-(cycloButylamino)-4-pyridyl]oxy]-2-fluoro-phenyl]carbamate

A mixture of tert-Butyl N-[4-[(2-chloro-4-pyridyl)oxy]-2-fluoro-phenyl]carbamate (300 mg, 885.58 umol, 1 eq), cyclobutylamine (114.33 mg, 1.06 mmol, 137.74 uL, 1.2 eq, HCl salt), Pd(dba)₂ (101.84 mg, 177.12 umol, 0.2 eq), XPhos (168.87 mg, 354.23 umol, 0.4 eq), Cs₂CO₃ (865.62 mg, 2.66 mmol, 3 eq) in dioxane (10 mL) was degassed and purged with N₂ for 3 times. The mixture was stirred at 90° C. for 4 h under N₂ atmosphere. LC-MS showed that the reactant was consumed completely and desired mass was detected. The residue was poured into water (30 mL) and stirred for 2 minutes. The aqueous phase was extracted with ethyl acetate (20 mL×2), dried with anhydrous Na₂SO₄, filtered and concentrated under reduced pressure to afford a crude residue which was purified by column chromatography (SiO₂, Petroleum ether/Ethyl acetate=3/1 to 1/1; TLC (Petroleum ether/Ethyl acetate=3/1)) to afford tert-Butyl N-[4-[[2-(cycloButylamino)-4-pyridyl]oxy]-2-fluoro-phenyl]carbamate (250 mg, 669.49 umol, 75.60% yield) as a yellow solid. LC-MS: [M+H]⁺=374.1.

### Step 2: Preparation of 4-(4-amino-3-fluoro-phenoxy)-N-cycloButyl-pyridin-2-amine

A mixture of tert-Butyl N-[4-[[2-(cycloButylamino)-4-pyridyl]oxy]-2-fluoro-phenyl]carbamate (200 mg, 535.59 umol, 1 eq) in HCl/dioxane (4 M, 10 mL, 74.68 eq) was stirred at 20° C. for 0.5 h. LC-MS indicated full consumption of the reactant and mass of the desired compound was detected. The mixture was concentrated under reduced pressure to give 4-(4-amino-3-fluoro-phenoxy)-N-cycloButyl-pyridin-2-amine (160 mg, 516.52 umol, 96.44% yield, HCl salt) as a white solid. LC-MS: [M+H]⁺=274.1.

### Step 3: Preparation of 1-[4-[4-[4-[[2-(cycloButylamino)-4-pyridyl]oxy]-2-fluoro-anilino]-7H-pyrrolo[2,3-d]pyrimidin-5-yl]-1-piperidyl]prop-2-en-1-one

To a solution of 4-(4-amino-3-fluoro-phenoxy)-N-cycloButyl-pyridin-2-amine (70 mg, 225.98 umol, 1 eq, HCl salt) in DMF/MeCN (1 mL/1 mL) and MeCN (1 mL) was added TsOH (77.83 mg, 451.95 umol, 2 eq) and 1-[4-(4-chloro-7H-pyrrolo[2,3-d]pyrimidin-5-yl)-1-piperidyl]prop-2-en-1-one. The mixture was stirred at 70° C. for 12 h. LC-MS analysis indicated that the reactant was consumed completely and the desired mass was detected. The reaction mixture was poured into sodium bicarbonate saturated solution (10 mL) and stirred for 1 minute. The aqueous phase was extracted with ethyl acetate (10 mL×2). The combined organic phase was washed with brine (10 mL), dried with anhydrous Na₂SO₄, filtered and concentrated under reduced pressure to give a crude residue which was purified by prep-HPLC (column: Phenomenex luna C18 150*25 mm*10 um; mobile phase: [water(FA)-ACN]; B %: 5%-35%, 2 min) to afford 1-[4-[4-[4-[[2-(cycloButylamino)-4-pyridyl]oxy]-2-fluoro-anilino]-7H-pyrrolo[2,3-d]pyrimidin-5-yl]-1-piperidyl]prop-2-en-1-one (6 mg, 10.84 umol, 4.80% yield, 95.3% purity) as a white solid. ¹H NMR (400 MHz, DMSO-d₆) δ 11.62 (br, 1H), 8.21 (s, 1H), 7.98-7.83 (m, 3H), 7.22-7.16 (m, 1H), 7.06 (s, 1H), 7.02 (d, J=8.8 Hz, 1H), 6.90-6.79 (m, 2H), 6.20-6.15 (m, 1H), 6.14-6.08 (m, 1H), 5.90 (d, J=1.6 Hz, 1H), 5.70-5.61 (m, 1H), 4.64-4.54 (m, 1H), 4.26-4.14 (m, 2H), 3.47-3.42 (m, 2H), 2.82 (m, 1H), 2.27-2.19 (m, 2H), 2.08 (m, 2H), 1.85-1.77 (m, 2H), 1.69-1.60 (m, 2H), 1.53-1.42 (m, 2H), LC-MS: [M+H]⁺=528.2.

### Example 13: Preparation of 1-[4-[4-[2-fluoro-4-[[2-(3-fluoro-3-methyl-pyrrolidin-1-yl)-4-pyridyl]oxy]anilino]-7H-pyrrolo[2,3-d]pyrimidin-5-yl]-1-piperidyl]prop-2-en-1-one, Compound 57

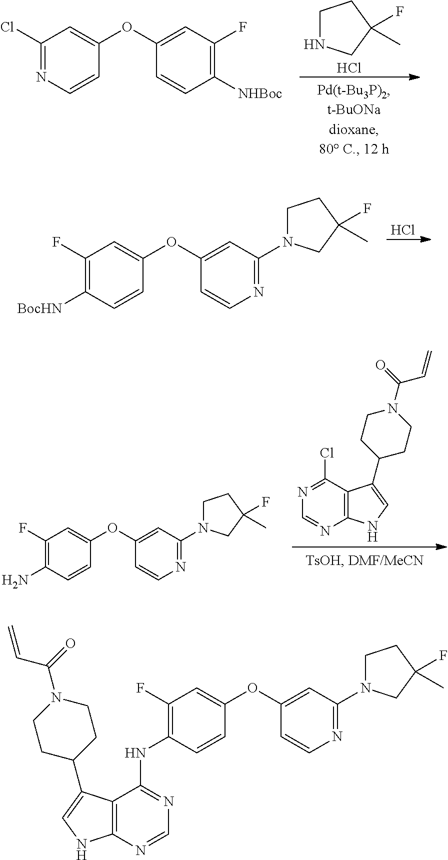

### Step 1: Preparation of tert-Butyl N-[2-fluoro-4-[[2-(3-fluoro-3-methyl-pyrrolidin-1-yl)-4-pyridyl]oxy]phenyl]carbamate

To a solution of tert-Butyl N-[4-[(2-chloro-4-pyridyl)oxy]-2-fluoro-phenyl]carbamate (300 mg, 885.58 umol, 1 eq) and 3-fluoro-3-methyl-pyrrolidine (222.53 mg, 1.59 mmol, 1.8 eq, HCl salt) in dioxane (10 mL) were added palladium; tritert-Butylphosphane (90.52 mg, 177.12 umol, 0.2 eq) and t-BuONa (255.31 mg, 2.66 mmol, 3 eq) at 20° C. under N₂. The mixture was stirred for 5 h at 80° C. under N₂. LC-MS analysis showed that tert-Butyl N-[4-[(2-chloro-4-pyridyl) oxy]-2-fluoro-phenyl] carbamate was fully consumed and the desired MS was detected. The mixture was concentrated to give a residue which was purified by column chromatography (SiO₂, Petroleum ether/Ethyl acetate=1/0 to 1/1) to get tert-Butyl N-[2-fluoro-4-[[2-(3-fluoro-3-methyl-pyrrolidin-1-yl)-4-pyridyl] oxy] phenyl] carbamate (160 mg, 394.64 umol, 44.56% yield) as brown oil. ¹H NMR (DMSO-d₆, 400 MHz) δ 8.94 (s, 1H), 7.97 (d, J=5.6 Hz, 1H), 7.59 (br t, J=6.0 Hz, 1H), 7.10-7.09 (m, 1H), 6.92 (d, J=8.0 Hz, 1H), 6.21-6.13 (m, 1H), 5.95 (s, 1H), 3.68-3.52 (m, 2H), 3.46-3.42 (m, 2H), 2.22-2.01 (m, 2H), 1.54-1.51 (m, 3H), 1.46 (s, 9H), LC-MS: [M+H]⁺=406.2.

### Step 2: Preparation of 2-fluoro-4-[[2-(3-fluoro-3-methyl-pyrrolidin-1-yl)-4-pyridyl]oxy]aniline

A mixture of tert-Butyl N-[2-fluoro-4-[[2-(3-fluoro-3-methyl-pyrrolidin-1-yl)-4-pyridyl] oxy]phenyl] carbamate (160 mg, 394.64 umol, 1 eq) in HCl/dioxane (4 M, 6 mL) was stirred for 12 h at 20° C. LC-MS analysis showed that tert-Butyl N-[2-fluoro-4-[[2-(3-fluoro-3-methyl-pyrrolidin-1-yl)-4-pyridyl] oxy] phenyl] carbamate was fully consumed and the desired mass was detected. The mixture was concentrated to give 2-fluoro-4-[[2-(3-fluoro-3-methyl-pyrrolidin-1-yl)-4-pyridyl]oxy] aniline (134 mg, 270.52 umol, 68.55% yield, 69% purity, HCl salt) as brown oil without further purification. LC-MS: [M+H]⁺=306.0

### Step 3: Preparation of 1-[4-[4-[2-fluoro-4-[[2-(3-fluoro-3-methyl-pyrrolidin-1-yl)-4-pyridyl]oxy]anilino]-7H-pyrrolo[2,3-d]pyrimidin-5-yl]-1-piperidyl]prop-2-en-1-one

To a solution of 2-fluoro-4-[[2-(3-fluoro-3-methyl-pyrrolidin-1-yl)-4-pyridyl]oxy]aniline (134 mg, 270.52 umol, 69% purity, 1 eq, HCl salt) and 1-[4-(4-chloro-7H-pyrrolo[2,3-d]pyrimidin-5-yl)-1-piperidyl]prop-2-en-1-one (70.79 mg, 243.47 umol, 0.9 eq) in DMF/MeCN (2 mL/2 mL) was added TsOH (139.75 mg, 811.57 umol, 3 eq) at 20° C. The mixture was stirred for 24 h at 70° C. LC-MS analysis showed that the desired mass was detected. The mixture was extracted with EA (40 mL×2). The combined organic layers were washed with brine (50 mL), dried over Na₂SO₄, filtered and concentrated under reduced pressure to give a residue which was purified by Prep-HPLC (neutral column: Waters Xbridge C18 150*50 mm*10 um; mobile phase: [water(NH₄HCOs)-ACN]; B %: 34%-64%, 10 min) to afford 1-[4-[4-[2-fluoro-4-[[2-(3-fluoro-3-methyl-pyrrolidin-1-yl)-4-pyridyl]oxy]anilino]-7H-pyrrolo[2,3-d]pyrimidin-5-yl]-1-piperidyl]prop-2-en-1-one (17 mg, 29.74 umol, 10.99% yield, 97.9% purity) as white solid. ¹H NMR (DMSO-d₆, 400 MHz) δ 11.61 (s, 1H), 8.16-8.12 (m, 1H), 8.03 (d, J=2.8 Hz, 1H), 7.93-7.85 (m, 2H), 7.21-7.16 (m, 1H), 7.07-6.99 (m, 2H), 6.88-6.79 (m, 1H), 6.24 (dd, J=2.0, 5.6 Hz, 1H), 6.14-6.07 (m, 1H), 6.04-5.99 (m, 1H), 5.71-5.56 (m, 1H), 4.62-4.55 (m, 1H), 4.22-4.11 (m, 1H), 3.73-3.64 (m, 1H), 3.63-3.54 (m, 1H), 3.49-3.36 (m, 4H), 2.86-2.77 (m, 1H), 2.25-2.15 (m, 2H), 2.12-2.01 (m, 2H), 1.52 (d, J=8.8 Hz, 3H), 1.50-1.43 (m, 2H), LC-MS: [M+H]⁺=560.3.

### Example 14: Preparation of 1-(4-(4-((2-fluoro-4-((2-(3-methoxy-3-methylazetidin-1-yl)pyridin-4-yl)oxy)phenyl)amino)-1H-pyrazolo[3,4-d]pyrimidin-3-yl)piperidin-1-yl)prop-2-en-1-one, Compound 178

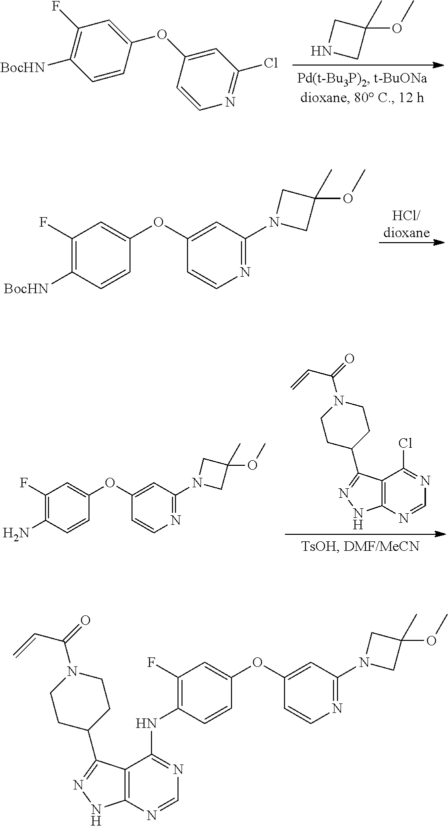

### Step 1: Preparation of Tert-Butyl (2-fluoro-4-((2-(3-methoxy-3-methylazetidin-1-yl)pyridin-4-yl)oxy)phenyl)carbamate

A mixture of tert-Butyl N-[4-[(2-chloro-4-pyridyl)oxy]-2-fluoro-phenyl]carbamate (250 mg, 737.98 umol, 1 eq), 3-methoxy-3-methyl-azetidine (121.86 mg, 885.58 umol, 1.2 eq, HCl), t-BuONa (212.77 mg, 2.21 mmol, 3 eq), dioxane (10 mL) was purged with N₂ for 3 times. Pd(t-Bu₃P)₂ (75.43 mg, 147.60 umol, 0.2 eq) was added, and purged with N₂ for 3 times again. The reaction mixture was stirred at 80° C. for 4 h. LC-MS analysis showed that desired mass was detected. The reaction mixture was quenched with H₂O (10 mL) at 20° C., and extracted with EA (10 mL×3). The combined organic layers were washed with brine (10 mL×3), dried over by Na₂SO₄, filtered and concentrated under reduced pressure to give a residue which was purified by column chromatography (SiO₂, Petroleum ether/Ethyl acetate=1/0 to 1/1). Tert-Butyl N-[2-fluoro-4-[[2-(3-methoxy-3-methyl-azetidin-1-yl)-4-pyridyl]oxy]phenyl]carbamate (200 mg, 485.81 umol, 32.91% yield, 98% purity) was obtained as a colorless oil. LC-MS: [M+H]⁺=404.2.

### Step 2: Preparation of 2-fluoro-4-((2-(3-methoxy-3-methylazetidin-1-yl)pyridin-4-yl)oxy)aniline

To a solution of tert-Butyl N-[2-fluoro-4-[[2-(3-methoxy-3-methyl-azetidin-1-yl)-4-pyridyl]oxy]phenyl]carbamate (200 mg, 495.73 umol, 1 eq) in dioxane (1 mL) was added HCl/dioxane (4 M, 3 mL, 24.21 eq) at 0° C. for 0.5 h. The mixture was stirred at 20° C. for 12 h. LC-MS analysis showed that desired mass was detected. The reaction mixture was concentrated under reduced pressure to give 2-fluoro-4-[[2-(3-methoxy-3-methyl-azetidin-1-yl)-4-pyridyl]oxy]aniline (150 mg, 441.45 umol, 89.05% yield, HCl) as a black brown solid without further purification. LC-MS: [M+H]⁺=304.1

### Step 3: Preparation of 1-(4-(4-((2-fluoro-4-((2-(3-methoxy-3-methylazetidin-1-yl)pyridin-4-yl)oxy)phenyl)amino)-1H-pyrazolo[3,4-d]pyrimidin-3-yl)piperidin-1-yl)prop-2-en-1-one

To a solution of 2-fluoro-4-[[2-(3-methoxy-3-methyl-azetidin-1-yl)-4-pyridyl]oxy]aniline (40 mg, 117.72 umol, 1 eq, HCl) in DMF (4 mL) was added TsOH (40.54 mg, 235.44 umol, 2 eq) and 1-[4-(4-chloro-1H-pyrazolo[3,4-d]pyrimidin-3-yl)-1-piperidyl]prop-2-en-1-one (41.21 mg, 141.26 umol, 1.2 eq). The mixture was stirred at 50° C. for 12 h. LC-MS analysis showed that the desired mass was detected. The reaction mixture was diluted with NaHCO₃ (saturated, 10 mL) and extracted with EA (5 mL×3). The combined organic layers were washed with brine (5 mL×3), dried over Na₂SO₄, filtered and concentrated under reduced pressure to give a residue which was purified by prep-HPLC (column: Phenomenex C18 75*30 mm*3 um; mobile phase: [water(FA)-ACN]; B %: 12%-42%, 7 min). 1-[4-[4-[2-fluoro-4-[[2-(3-methoxy-3-methyl-azetidin-1-yl)-4-pyridyl]oxy]anilino]-1H-pyrazolo[3,4-d]pyrimidin-3-yl]-1-piperidyl]prop-2-en-1-one (14.18 mg, 23.45 umol, 19.92% yield, 100% purity, FA) was obtained as a white solid. ¹H NMR (DMSO-d₆, 400 MHz) δ 13.37 (s, 1H), 8.60-8.67 (m, 1H), 8.21-8.26 (m, 1H), 8.03 (d, J=6.00 Hz, 1H), 7.63-7.71 (m, 1H), 7.21-7.26 (m, 1H), 7.04-7.09 (m, 1H), 6.82-6.92 (m, 1H), 6.29 (dd, J=8.00, 3.60 Hz, 1H), 6.12 (dd, J=19.20, 14.4 Hz, 1H), 5.97 (d, J=2.00 Hz, 1H), 5.66-5.71 (m, 1H), 4.51 (m, 1H), 4.17 (m, 1H), 3.84 (m, 2H) 3.73 (m, 2H) 3.30 (s, 2H) 3.19 (s, 3H) 2.92 (m, 1H) 2.07 (m, 2H) 1.67 (s, 2H) 1.46 (s, 3H), LC-MS: [M+H]⁺=559.2.

### Example 15: Preparation of 1-(4-(4-((2-fluoro-4-((2-(3-methoxy-3-methylazetidin-1-yl)pyridin-4-yl)oxy)phenyl)amino)pyrrolo[2,1-f][1,2,4]triazin-5-yl)piperidin-1-yl)prop-2-en-1-one, Compound 203

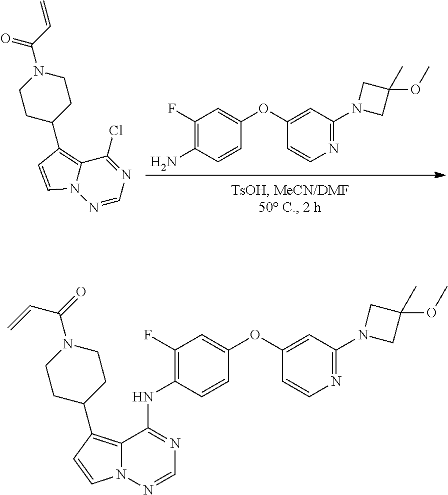 [SMILES: C=CC(=O)N1CCC(c2ccn3ncnc(Cl)c23)CC1 ; COC1(C)CN(c2cc(Oc3ccc(N)c(F)c3)ccn2)C1 ; C=CC(=O)N1CCC(c2ccn3ncnc(Nc4ccc(Oc5ccnc(N6CC(C)(OC)C6)c5)cc4F)c23)CC1]

To a solution of 2-fluoro-4-[[2-(3-methoxy-3-methyl-azetidin-1-yl)-4-pyridyl]oxy]aniline (30 mg, 88.29 umol, 1 eq, HCl) and 1-[4-(4-chloropyrrolo[2,1-f][1,2,4]triazin-5-yl)-1-piperidyl]prop-2-en-1-one (25.67 mg, 88.29 umol, 1 eq) in MeCN 4 mL was added TsOH (15.20 mg, 88.29 umol, 1 eq). The mixture was stirred at 50° C. for 2 hr. LC-MS showed that desired mass was detected. The reaction mixture was diluted with saturated NaHCO₃ solution (10 mL) and extracted with EA (10 mL×3). The combined organic layers were washed with brine (10 mL×3), dried over Na₂SO₄, filtered and concentrated under reduced pressure to give a residue which was purified by prep-HPLC (column: Phenomenex C18 75*30 mm*3 um; mobile phase: [water(FA)-ACN]; B %: 15%-45%, 7 min) and was further purified by prep-HPLC (column: 3_Phenomenex Luna C18 75*30 mm*3 um; mobile phase: [water(TFA)-ACN]; B %: 29%-49%, 9 min). 1-[4-[4-[2-fluoro-4-[[2-(3-methoxy-3-methyl-azetidin-1-yl)-4-pyridyl]oxy]anilino]pyrrolo[2,1-f][1,2,4]triazin-5-yl]-1-piperidyl]prop-2-en-1-one (4.82 mg, 7.18 umol, 8.13% yield, 100% purity, TFA) was obtained as a white solid. ¹H NMR (CD₃OD 400 MHz) δ 7.90 (br d, J=7.21 Hz, 2H), 7.71 (s, 1H), 7.58 (d, J=2.40 Hz, 1H), 7.22-7.28 (m, 1H), 7.11-7.18 (m, 1H), 6.83 (dd, J=27.60, 6.40 Hz, 1H), 6.69 (s, 2H), 6.19-6.25 (m, 1H), 6.16-6.18 (m, 1H), 5.74-5.80 (m, 1H), 4.69-4.77 (m, 1H), 4.23-4.33 (m, 1H), 4.18 (d, J=9.20 Hz, 2H), 3.99-4.11 (m, 2H), 3.56-3.66 (m, 1H), 3.34-3.41 (m, 1H), 3.31 (s, 3H), 2.87-2.99 (m, 1H), 2.09-2.21 (m, 2H), 1.67-1.84 (m, 2H), 1.57 (s, 3H), LCMS: [M+H]⁺=558.1.

### Example 16: Preparation of 1-[4-[4-[2-fluoro-4-[4-(5-fluoro-6-methyl-3-pyridyl)thiazol-2-yl]oxy-anilino]-1H-pyrazolo[3,4-d]pyrimidin-3-yl]-1-piperidyl]prop-2-en-1-one, Compound 190

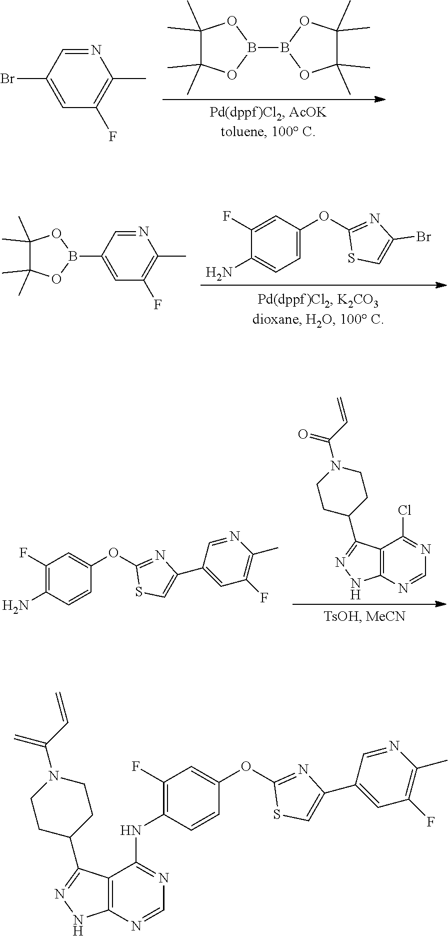

### Step 1: Preparation of 3-fluoro-2-methyl-5-(4,4,5,5-tetramethyl-1,3,2-dioxaborolan-2-yl)pyridine

A mixture of 5-bromo-3-fluoro-2-methyl-pyridine (2 g, 10.53 mmol, 1 eq), 4,4,5,5-tetramethyl-2-(4,4,5,5-tetramethyl-1,3,2-dioxaborolan-2-yl)-1,3,2-dioxaborolane (2.94 g, 11.58 mmol, 1.1 eq), Pd (dppf) Cl₂ (770.16 mg, 1.05 mmol, 0.1 eq) and AcOK (3.10 g, 31.58 mmol, 3 eq) in toluene (50 mL) was degassed and purged with N₂ for 3 times. The mixture was stirred at 100° C. for 12 h under N₂ atmosphere. TLC (PE:EA=4:1) indicated the bromide reactant was consumed completely and two new spots formed. The reaction mixture was concentrated under reduced pressure to give a residue. The residue was purified by column chromatography (SiO₂, Petroleum ether/Ethyl acetate=100:1 to 30:1) to get 3-fluoro-2-methyl-5-(4,4,5,5-tetramethyl-1,3,2-dioxaborolan-2-yl)pyridine (2.4 g, 10.12 mmol, 96% yield) as yellow oil. ¹H NMR (CDCl₃ 400 MHz) δ 8.61 (s, 1H), 7.65 (d, J=9.6 Hz, 1H), 2.54 (d, J=2.8 Hz, 3H), 1.34 (s, 12H)

### Step 2: Preparation of 2-fluoro-4-[4-(5-fluoro-6-methyl-3-pyridyl)thiazol-2-yl]oxy-aniline

A mixture of 3-fluoro-2-methyl-5-(4,4,5,5-tetramethyl-1,3,2-dioxaborolan-2-yl)pyridine (491.99 mg, 2.08 mmol, 1.2 eq), 4-(4-bromothiazol-2-yl)oxy-2-fluoro-aniline (500 mg, 1.73 mmol, 1 eq), Pd(dppf)Cl₂·CH₂Cl₂ (282.45 mg, 345.87 umol, 0.2 eq) and K₂CO₃ (717.05 mg, 5.19 mmol, 3 eq) in dioxane (20 mL) and H₂O (2 mL) was degassed and purged with N₂ for 3 times. The mixture was stirred at 90° C. for 1 h under N₂ atmosphere. LC-MS analysis indicated that 4-(4-bromothiazol-2-yl)oxy-2-fluoro-aniline was consumed completely and one main peak with the desired mass was detected. TLC (PE:EA=2:1) indicated that 4-(4-bromothiazol-2-yl)oxy-2-fluoro-aniline was consumed completely and two new spots formed. The reaction mixture was concentrated under reduced pressure to give a residue which was purified by column chromatography (SiO₂, Petroleum ether/Ethyl acetate=100:1 to 30:1) to get 2-fluoro-4-[4-(5-fluoro-6-methyl-3-pyridyl)thiazol-2-yl]oxy-aniline (550 mg, 1.72 mmol, 99% yield) as yellow oil. ¹H NMR (DMSO-d₆ 400 MHz,) δ 8.78 (s, 1H), 8.00 (dd, J=1.6, 10.8 Hz, 1H), 7.76 (s, 1H), 7.24 (dd, J=2.8, 11.6 Hz, 1H), 7.00 (dd, J=2.0, 8.8 Hz, 1H), 6.89-6.77 (m, 1H), 5.30 (br S, 1H), 1.46 (d, J=2.8 Hz, 3H), LC-MS: [M+H]⁺=320.1.

### Step 3: Preparation of 1-[4-[4-[2-fluoro-4-[4-(5-fluoro-6-methyl-3-pyridyl)thiazol-2-yl]oxy-anilino]-1H-pyrazolo[3,4-d]pyrimidin-3-yl]-1-piperidyl]prop-2-en-1-one

To a solution of 2-fluoro-4-[4-(5-fluoro-6-methyl-3-pyridyl)thiazol-2-yl]oxy-aniline (218.92 mg, 685.55 umol, 1 eq) in DMF (2 mL) was added TsOH (118.05 mg, 685.55 umol, 1 eq) and 1-[4-(4-chloro-1H-pyrazolo[3,4-d]pyrimidin-3-yl)-1-piperidyl]prop-2-en-1-one (200 mg, 685.55 umol, 1 eq). The mixture was stirred at 50° C. for 12 h. LC-MS analysis showed that 2-fluoro-4-[4-(5-fluoro-6-methyl-3-pyridyl)thiazol-2-yl]oxy-aniline was consumed completely and desired mass was detected. The reaction mixture was quenched by NaHCO₃ (sat., 30 mL) at 25° C., diluted with EA (10 mL) and extracted with EA (15 mL×2). The combined organic layers were washed with water (15 mL×2), dried over Na₂SO₄, filtered and concentrated under reduced pressure to give a residue which was purified by prep-HPLC (neutral condition; column: Waters Xbridge 150*25 mm*5 um; mobile phase: [water(NH₄HCOs)-ACN]; B %: 35%-65%, 10 min) and lyophilized to afford 1-[4-[4-[2-fluoro-4-[4-(5-fluoro-6-methyl-3-pyridyl)thiazol-2-yl]oxy-anilino]-1H-pyrazolo[3,4-d]pyrimidin-3-yl]-1-piperidyl]prop-2-en-1-one (72 mg, 119.04 umol, 17% yield, 95% purity) as a white solid. ¹H NMR (DMSO-d₆, 400 MHz) δ 13.39 (br s, 1H), 8.80 (s, 1H), 8.70 (s, 1H), 8.25 (s, 1H), 8.03 (br d, J=10.8 Hz, 1H), 7.90 (s, 1H), 7.79-7.70 (m, 1H), 7.62 (dd, J=2.4, 10.4 Hz, 1H), 7.41 (br d, J=8.8 Hz, 1H), 6.86 (dd, J=10.8, 16.8 Hz, 1H), 6.11 (dd, J=2.4, 16.8 Hz, 1H), 5.68 (dd, J=2.4, 10.4 Hz, 1H), 4.57-4.46 (m, 1H), 4.25-4.15 (m, 1H), 3.77-3.67 (m, 1H), 3.33-3.25 (m, 1H), 2.90-2.80 (m, 1H), 2.47 (s, 3H), 2.13-2.01 (m, 2H), 1.73-1.58 (m, 2H), LC-MS: [M+H]⁺=575.2.

### Example 17: Preparation of 1-[4-[4-[2-fluoro-4-[4-(5-fluoro-6-methyl-3-pyridyl)thiazol-2-yl]oxy-anilino]pyrrolo[2,1-f][1,2,4]triazin-5-yl]-1-piperidyl]prop-2-en-1-one, Compound 206

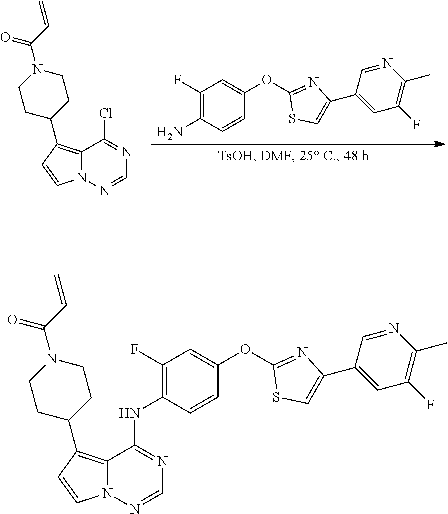 [SMILES: C=CC(=O)N1CCC(c2ccn3ncnc(Cl)c23)CC1 ; Cc1ncc(-c2csc(Oc3ccc(N)c(F)c3)n2)cc1F ; C=CC(=O)N1CCC(c2ccn3ncnc(Nc4ccc(Oc5nc(-c6cnc(C)c(F)c6)cs5)cc4F)c23)CC1]

To a solution of 1-[4-(4-chloropyrrolo[2,1-f][1,2,4]triazin-5-yl)-1-piperidyl]prop-2-en-1-one (200 mg, 687.88 umol, 1 eq) DMF (2 mL) was added TsOH (118.45 mg, 687.88 umol, 1 eq) and 2-fluoro-4-[4-(5-fluoro-6-methyl-3-pyridyl)thiazol-2-yl]oxy-aniline (219.66 mg, 687.88 umol, 1 eq). The mixture was stirred at 25° C. for 48 h. LC-MS analysis showed that 1-[4-(4-chloropyrrolo[2,1-f][1,2,4]triazin-5-yl)-1-piperidyl]prop-2-en-1-one was consumed completely and desired mass was detected. The reaction mixture was quenched with NaHCO₃ (sat. 30 mL) at 25° C., and then diluted with EA (10 mL) and extracted with EA (15 mL×2). The combined organic layers were washed with water (15 mL×2), dried over Na₂SO₄, filtered and concentrated under reduced pressure to give a residue which was purified by prep-HPLC (neutral condition; column: Waters Xbridge 150*25 mm*5 um; mobile phase: [water(NH₄HCO₃)-ACN]; B %: 48%-78%, 9 min) and lyophilized to afford 1-[4-[4-[2-fluoro-4-[4-(5-fluoro-6-methyl-3-pyridyl)thiazol-2-yl]oxy-anilino]pyrrolo[2,1-f][1,2,4]triazin-5-yl]-1-piperidyl]prop-2-en-1-one (74 mg, 126.94 umol, 18% yield, 98.4% purity) as a white solid. ¹H NMR (DMSO-d₆ 400 MHz,) δ 8.80 (s, 1H), 8.02 (dd, J=1.6, 10.8 Hz, 1H), 7.89 (s, 1H), 7.86-7.79 (m, 1H), 7.77-7.66 (m, 1H), 7.64-7.53 (m, 1H), 7.51-7.03 (m, 2H), 6.89-6.81 (m, 1H), 6.71 (br d, J=2.8 Hz, 1H), 6.11 (d, J=16.8 Hz, 1H), 5.67 (dd, J=2.4, 10.4 Hz, 1H), 4.60-4.55 m, 1H), 4.23-4.09 (m, 1H), 3.72-3.55 (m, 1H), 3.28-3.07 (m, 1H), 2.95-2.69 (m, 1H), 2.47 (d, J=2.8 Hz, 3H), 1.98 (d, J=7.6 Hz, 2H), 1.62-1.49 (m, 2H), LC-MS: [M+H]⁺=574.1.

### Example 18: Preparation of 1-[4-[4-[3-chloro-4-(2-pyridylmethoxy) anilino]thieno[2,3-d]pyrimidin-5-yl]-1-piperidyl]prop-2-en-1-one, Compound 160

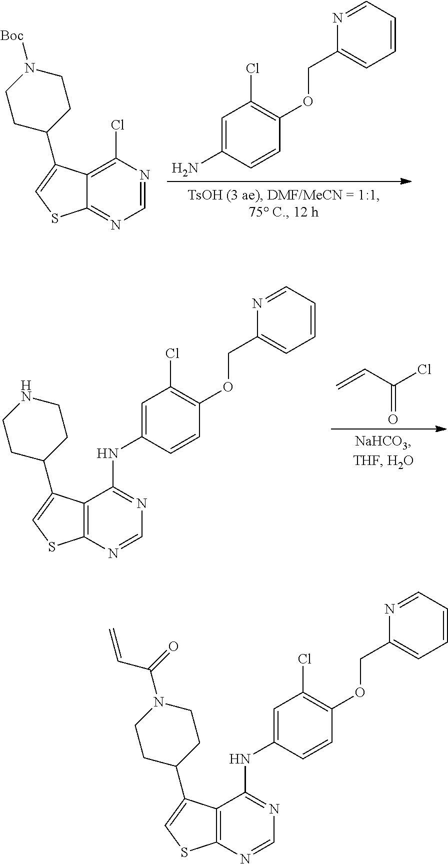

### Step 1: Preparation of N-[3-chloro-4-(2-pyridylmethoxy)phenyl]-5-(4-piperidyl)thieno[2,3-d]pyrimidin-4-amine

To a solution of tert-Butyl 4-(4-chlorothieno[2,3-d]pyrimidin-5-yl)piperidine-1-carboxylate (250 mg, 673.98 umol, 95.4% purity, 1 eq) in MeCN (5 mL) and DMF (5 mL) was added 3-chloro-4-(2-pyridylmethoxy)aniline (158.17 mg, 673.98 umol, 1 eq), TsOH (348.18 mg, 2.02 mmol, 3 eq). The mixture was stirred at 75° C. for 12 h. LC-MS showed that tert-Butyl 4-(4-chlorothieno [2,3-d]pyrimidin-5-yl)piperidine-1-carboxylate was consumed and the desired mass was detected. The mixture was poured into saturated aqueous NaHCO₃ (50 mL), extracted with EA (30 mL×3). The combined organic layer was washed with brine (50 mL), dried over Na₂SO₄, filtered and concentrated to give N-[3-chloro-4-(2-pyridylmethoxy)phenyl]-5-(4-piperidyl)thieno[2,3-d]pyrimidin-4-amine (0.52 g, 1.02 mmol, 75.96% yield, 89% purity) as yellow solid. LC-MS: [M+H]⁺=452.0.

### Step 2: Preparation of 1-[4-[4-[3-chloro-4-(2-pyridylmethoxy) anilino]thieno[2,3-d]pyrimidin-5-yl]-1-piperidyl]prop-2-en-1-one

To a solution of N-[3-chloro-4-(2-pyridylmethoxy)phenyl]-5-(4-piperidyl)thieno[2,3-d]pyrimidin-4-amine (0.52 g, 1.02 mmol, 89% purity, 1 eq) in THF (10 mL) and H₂O (10 mL) was added NaHCO₃ (430.10 mg, 5.12 mmol, 199.12 uL, 5 eq), prop-2-enoyl chloride (101.94 mg, 1.13 mmol, 91.84 uL, 1.1 eq) at 0° C. The mixture was stirred at 20° C. for 20 minutes. LC-MS analysis showed that N-[3-chloro-4-(2-pyridylmethoxy) phenyl]-5-(4-piperidyl)thieno[2,3-d]pyrimidin-4-amine was fully consumed and desired mass was formed. The mixture was poured into H₂O (30 mL), extracted with EA (30 mL×3), the combined organic layers were washed with brine (50 mL), dried over Na₂SO₄, filtered and concentrated to give a residue, which was purified by prep-HPLC (FA, column: Phenomenex luna C18 150*40 mm-15 um; mobile phase: [water(FA)-ACN]; B %: 33%-63%, 10 min), lyophilized to give 1-[4-[4-[3-chloro-4-(2-pyridylmethoxy)anilino]thieno[2,3-d]pyrimidin-5-yl]-1-piperidyl]prop-2-en-1-one (239.09 mg, 469.66 umol, 45.87% yield, 99.4% purity) as an off-white solid. ¹H NMR (DMSO-d₆, 400 MHz) δ 8.64-8.57 (m, 1H), 8.42 (s, 1H), 8.38 (s, 1H), 7.91-7.85 (m, 1H), 7.80-7.76 (m, 1H), 7.58 (d, J=7.8 Hz, 1H), 7.51-7.45 (m, 1H), 7.40 (s, 1H), 7.39-7.34 (m, 1H), 7.25 (d, J=8.8 Hz, 1H), 6.91-6.75 (m, 1H), 6.16-6.04 (m, 1H), 5.70-5.63 (m, 1H), 5.31 (s, 2H), 4.60-4.56 (m, 1H), 4.19-4.15 (m, 1H), 3.75-3.68 (m, 1H), 3.30-3.23 (m, 1H), 2.88-2.82 (m, 1H), 2.11-2.08 (m, 2H), 1.58-1.41 (m, 2H), LC-MS: [M+H]⁺=506.2.

### Example 19: Preparation of 1-(4-(4-((2-fluoro-4-((2-(2-methylthiazol-4-yl)pyridin-4-yl)oxy)phenyl)amino)-1H-pyrazolo[3,4-d]pyrimidin-3-yl)piperidin-1-yl)prop-2-en-1-one, Compound 171

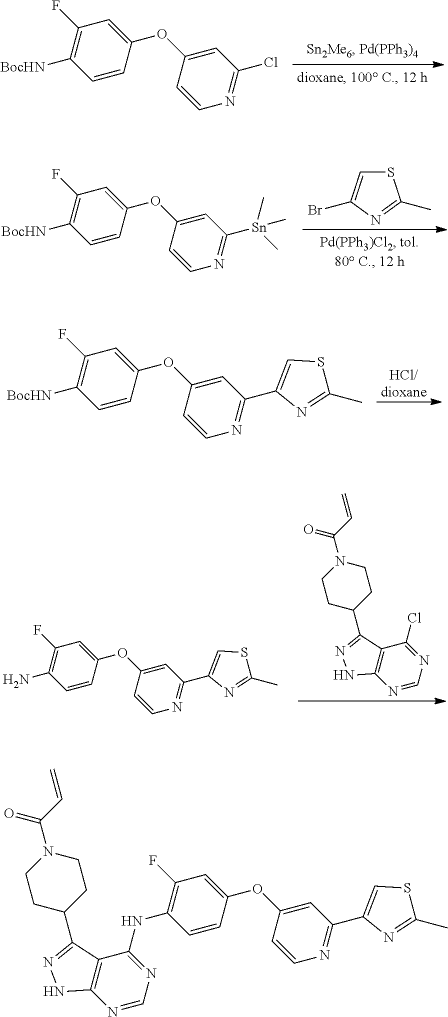

### Step 1: Preparation of tert-Butyl (4-((2-chloropyridin-4-yl)oxy)-2-fluorophenyl)carbamate

To a solution of tert-Butyl N-[4-[(2-chloro-4-pyridyl)oxy]-2-fluoro-phenyl]carbamate (1 g, 2.95 mmol, 1 eq) and trimethyl(trimethylstannyl)stannane (1.58 g, 4.82 mmol, 998.73 uL, 1.63 eq) in dioxane (10 mL) was added Pd(PPh₃)₄ (341.11 mg, 295.19 umol, 0.1 eq) at 20° C. under N₂. The mixture was stirred for 12 h at 110° C. under N₂. LC-MS analysis showed that tert-Butyl N-[4-[(2-chloro-4-pyridyl)oxy]-2-fluoro-phenyl]carbamate was fully consumed and the desired MS was detected. The mixture was concentrated to afford tert-Butyl N-[2-fluoro-4-[(2-trimethylstannyl-4-pyridyl)oxy]phenyl]carbamate (1.38 g, 815.38 umol, 27.62% yield, 27.6% purity) as black oil. LC-MS: [M+H]⁺=467.2

### Step 2: Preparation of tert-Butyl (2-fluoro-4-((2-(2-methylthiazol-4-yl)pyridin-4-yl)oxy)phenyl)carbamate

To a solution of tert-Butyl N-[2-fluoro-4-[(2-trimethylstannyl-4-pyridyl)oxy]phenyl]carbamate (1.38 g, 815.38 umol, 1 eq) and 4-bromo-2-methyl-thiazole (290.36 mg, 1.63 mmol, 2 eq) in toluene (10 mL) was added Pd(PPh₃)₂Cl₂ (57.23 mg, 81.54 umol, 0.1 eq) at 20° C. under N₂. The mixture was stirred for 24 h at 80° C. under N₂. LC-MS showed that tert-Butyl N-[2-fluoro-4-[(2-trimethylstannyl-4-pyridyl)oxy]phenyl]carbamate was consumed and the desired MS was detected. The mixture was concentrated to give a crude residue which was purified by Prep-HPLC (TFA column: Phenomenex luna C18 150*40 mm*15 um; mobile phase: [water(TFA)-ACN]; B %: 25%-55%, 10 min) to get tert-Butyl N-[2-fluoro-4-[[2-(2-methylthiazol-4-yl)-4-pyridyl]oxy]phenyl]carbamate (85 mg, 211.73 umol, 25.97% yield) as yellow solid. ¹H NMR (DMSO-d₆, 400 MHz) δ 9.11 (br s, 1H), 8.54 (d, J=5.6 Hz, 1H), 8.31 (s, 1H), 7.75-7.68 (m, 1H), 7.63 (s, 1H), 7.30 (dd, J=2.4, 11.2 Hz, 1H), 7.08-7.05 (m, 2H), 2.72 (s, 3H), 1.47 (s, 9H), LC-MS: [M+H]⁺=402.2

### Step 3: Preparation of 2-fluoro-4-((2-(2-methylthiazol-4-yl)pyridin-4-yl)oxy)aniline

To a mixture of tert-Butyl N-[2-fluoro-4-[[2-(2-methylthiazol-4-yl)-4-pyridyl]oxy]phenyl]carbamate (80 mg, 199.28 umol, 1 eq) in HCl/dioxane (4 M, 6 mL, 120.44 eq) was stirred for 3 h at 20° C. LC-MS analysis indicated that the desired MS was detected. The mixture was concentrated to afford 2-fluoro-4-[[2-(2-methylthiazol-4-yl)-4-pyridyl]oxy]aniline (65 mg, 193.70 umol, 97.20% yield) as yellow solid, which was used for next step without further purification. LC-MS: [M+H]⁺=302.2.

### Step 4: Preparation of 1-(4-(4-((2-fluoro-4-((2-(2-methylthiazol-4-yl)pyridin-4-yl)oxy)phenyl)amino)-1H-pyrazolo[3,4-d]pyrimidin-3-yl)piperidin-1-yl)prop-2-en-1-one

To a solution of 1-[4-(4-chloro-1H-pyrazolo[3,4-d]pyrimidin-3-yl)-1-piperidyl]prop-2-en-1-one (45 mg, 154.25 umol, 1 eq) and 2-fluoro-4-[[2-(2-methylthiazol-4-yl)-4-pyridyl]oxy]aniline (57.89 mg, 154.25 umol, 1 eq, HCl) in DMF (3 mL) was added TsOH (13.28 mg, 77.12 umol, 0.5 eq) at 20° C. The mixture was stirred for 12 h at 50° C. LC-MS analysis showed that 1-[4-(4-chloro-1H-pyrazolo[3,4-d]pyrimidin-3-yl)-1-piperidyl]prop-2-en-1-one was consumed and the desired MS was detected. The mixture was poured into NaHCO₃ (sat., 50 mL) and filtered. The filter cake was redissolved in THF (10 mL) and concentrated under reduced pressure. The residue was purified by Prep-HPLC (column: Phenomenex Synergi C18 150*25 mm*10 um; mobile phase: [water(FA)-ACN]; B %: 20%-47%, 9 min and FA column: Phenomenex Synergi C18 150*25 mm*10 um; mobile phase: [water(FA)-ACN]; B %: 22%-46%, 8 min) to afford 1-[4-[4-[2-fluoro-4-[[2-(2-methylthiazol-4-yl)-4-pyridyl]oxy]anilino]-1H-pyrazolo[3,4-d]pyrimidin-3-yl]-1-piperidyl]prop-2-en-1-one (15 mg, 25.79 umol, 16.72% yield, 95.7% purity) as white solid. ¹H NMR (DMSO-d₆, 400 MHz) δ 13.33 (s, 1H), 8.67 (s, 1H), 8.56 (d, J=5.6 Hz, 1H), 8.26 (s, 1H), 8.15 (s, 1H), 7.76-7.70 (m, 1H), 7.54 (d, J=2.8 Hz, 1H), 7.38 (dd, J=2.4, 10.8 Hz, 1H), 7.17 (dd, J=1.6, 8.4 Hz, 1H), 7.05 (dd, J=2.4, 5.6 Hz, 1H), 6.89-6.82 (m, 1H), 6.12 (dd, J=2.4, 16.8 Hz, 1H), 5.68 (dd, J=2.4, 10.6 Hz, 1H), 4.52-4.49 (m, 1H), 4.18-4.13 (m, 1H), 3.80-3.66 (m, 1H), 3.35-3.34 (m, 1H), 3.02-2.83 (m, 1H), 2.70 (s, 3H), 2.12-2.06 (m, 2H), 1.75-1.59 (m, 2H), LC-MS: [M+H]⁺=557.1.

### Example 20: Preparation of triisopropyl-[2-[6-(methoxymethoxy)-8-(4,4,5,5-tetramethyl-1,3,2-dioxaborolan-2-yl)-1-naphthyl]ethynyl]silane

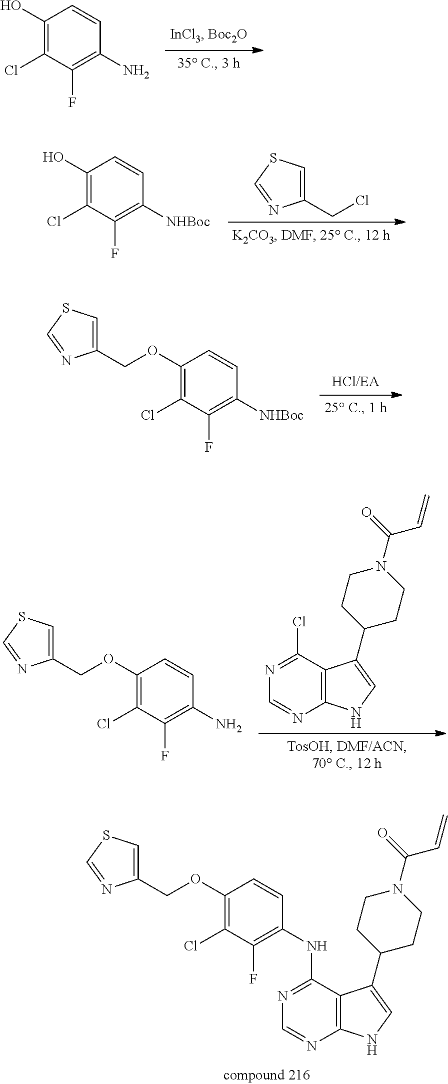

### Step 1: Preparation of tert-butyl (3-chloro-2-fluoro-4-hydroxyphenyl) carbamate

A mixture of 4-amino-2-chloro-3-fluoro-phenol (1.00 g, 6.19 mmol, 1 eq), Boc₂O (1.42 g, 6.50 mmol, 1.49 mL, 1.05 eq), InCl₃ (27. mg, 12 μmol, 7.91 μL, 0.02 eq) was stirred at 35° C. for 3 hours. TLC (PE/EA-5:1) indicated a new spot with lower polarity was detected. The reaction mixture was concentrated under reduced pressure to give a residue. The residue was purified by column chromatography (SiO₂, Petroleum ether/Ethyl acetate=1/0 to 5/1) to give tert-butyl (3-chloro-2-fluoro-4-hydroxyphenyl) carbamate (1.3 g, 4.97 mmol, 80.3% yield) as a white solid. ¹H NMR (400 MHz, DMSO-d₆) δ=10.48 (s, 1H), 8.74 (br s, 1H), 7.16 (br t, J=8.8 Hz, 1H), 6.83-6.66 (m, 1H), 1.43 (s, 9H).

### Step 2: Preparation of 3-(methoxymethoxy)-8-(2-triisopropylsilylethynyl)naphthalen-1-ol

A mixture of tert-butyl N-(3-chloro-2-fluoro-4-hydroxy-phenyl) carbamate (1.2 g, 4.59 mmol, 1 eq), 4-(chloromethyl)thiazole (819 mg, 4.82 mmol, 1.05 eq, HCl) and K₂CO₃ (1.90 g, 13.8 mmol, 3 eq) in DMF (10 mL) was stirred at 25° C. for 12 hours. LCMS showed 77% desired mass was detected. The reaction mixture was diluted with H₂O (30 mL) and extracted with EA (30 mL*3). The combined organic layers were washed with brine (30 mL*3), dried over Na₂SO₄, filtered, and concentrated under reduced pressure to give a residue. The residue was purified by column chromatography (SiO₂, Petroleum ether/Ethyl acetate=1/0 to 3/1) to give 3-(methoxymethoxy)-8-(2-triisopropylsilylethynyl)naphthalen-1-ol (1.6 g, 4.46 mmol, 97.2% yield) as a pink solid.

### Step 3: Preparation of 3-chloro-2-fluoro-4-(thiazol-4-ylmethoxy)aniline

To a solution of tert-butyl N-[3-chloro-2-fluoro-4-(thiazol-4-ylmethoxy) phenyl] carbamate (1.5 g, 4.18 mmol, 1 eq) in EtOAc (1 mL) was added HCl/EA (4 M, 15 mL, 14.35 eq). The mixture was stirred at 25° C. for 1 hour. LCMS showed 83% desired mass was detected. The reaction mixture was concentrated under reduced pressure to give a residue. The residue was dissolved in MeOH (10 mL), and MTBE (50 mL) was added slowly until the target product separated out. Filtered to give 3-chloro-2-fluoro-4-(thiazol-4-ylmethoxy) aniline (750 mg, 2.54 mmol, 60.8% yield, HCl) as a white solid.

### Step 4: Preparation of triisopropyl-[2-[6-(methoxymethoxy)-8-(4,4,5,5-tetramethyl-1,3,2-dioxaborolan-2-yl)-1-naphthyl]ethynyl]silane

A mixture of 3-chloro-2-fluoro-4-(thiazol-4-ylmethoxy)aniline (370 mg, 1.25 mmol, 1 eq, HCl salt), 1-[4-(4-chloro-7H-pyrrolo[2,3-d]pyrimidin-5-yl)-1-piperidyl]prop-2-en-1-one (364 mg, 1.25 mmol, 1 eq) and TsOH (648 mg, 3.76 mmol, 3 eq) in DMF (4 mL) and MeCN (4 mL) was stirred at 70° C. for 12 hours. TLC (PE:EA=0:1, RF=0.1) showed a new spot was detected. The reaction mixture was diluted with NaHCO₃ saturated solution 200 mL and extracted with EA (50 mL*3). The combined organic layers were washed with brine (100 mL*3), dried over by Na₂SO₄, filtered and concentrated under reduced pressure to give a residue. The residue was purified by prep-TLC (SiO₂, PE:EA=0:1, two times) to get the crude product. The crude product was purified by prep-HPLC (column: Waters xbridge 150*25 mm 10 μm; mobile phase: [water (NH₄HCOs)-ACN]; B %: 29%-59%, 10 min) to give triisopropyl-[2-[6-(methoxymethoxy)-8-(4,4,5,5-tetramethyl-1,3,2-dioxaborolan-2-yl)-1-naphthyl]ethynyl]silane (110 mg, 211 μmol, 8.43% yield, 98.6% purity) as a white solid. ¹H NMR (400 MHz, DMSO-d₆) δ=11.58 (d, J=2.0 Hz, 1H), 9.16 (d, J=2.0 Hz, 1H), 8.07 (s, 1H), 7.96 (s, 1H), 7.86 (d, J=2.0 Hz, 1H), 7.54 (t, J=8.8 Hz, 1H), 7.28-7.18 (m, 1H), 7.04 (d, J=2.0 Hz, 1H), 6.92-6.78 (m, 1H), 6.18-6.05 (m, 1H), 5.71-5.63 (m, 1H), 5.39 (s, 2H), 4.57 (br d, J=12.0 Hz, 1H), 4.21-4.10 (m, 1H), 3.48-3.41 (m, 1H), 3.30-3.21 (m, 1H), 2.88-2.75 (m, 1H), 2.12-1.99 (m, 2H), 1.55-1.37 (m, 2H).

### Example 21: Preparation of 1-(4-(4-((3-chloro-4-((2-methylthiazol-4-yl)methoxy)phenyl)amino)-7H-pyrrolo[2,3-d]pyrimidin-5-yl)piperidin-1-yl)prop-2-en-1-one (Compound 217)

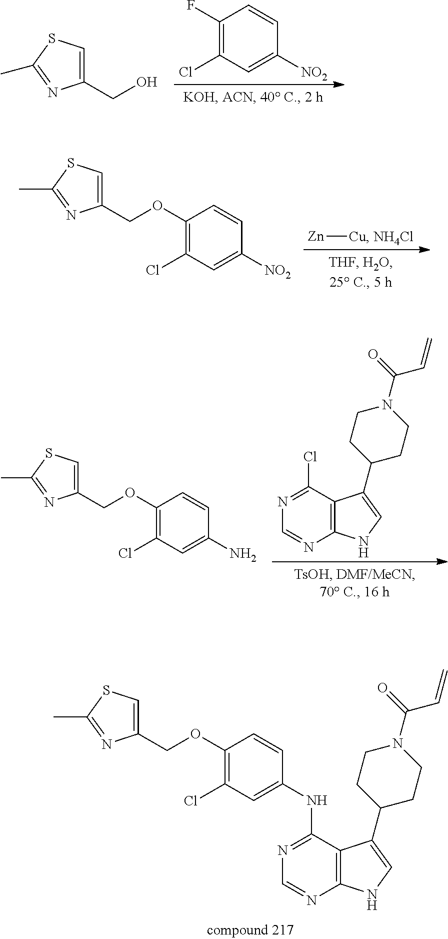

### Step 1:4-((2-chloro-4-nitrophenoxy)methyl)-2-methythiazole

To a solution of (2-methylthiazol-4-yl)methanol (515 mg, 3.99 mmol, 1 eq) in ACN (5 mL) was added 2-chloro-1-fluoro-4-nitro-benzene (700 mg, 3.99 mmol, 1 eq) and KOH (447 mg, 7.98 mmol, 2 eq). The mixture was stirred at 40° C. for 2 hours. A new peak was shown on LC-MS and 97% of desired compound was detected. TLC (PE:EA=5:1, RF=0.5) indicated one new spot formed. The reaction was clean. The reaction mixture was diluted with H₂O (50 mL) extracted with EA (20 mL*3). The combined organic layers were washed with brine (20 mL*3) and dried over Na₂SO₄, filtered and concentrated under reduced pressure to give 4-[(2-chloro-4-nitro-phenoxy)methyl]-2-methyl-thiazole (1.1 g, crude) as a yellow solid.

### Step 2: 3-chloro-4-((2-methylthiazol-4-yl)methoxy)aniline

To a solution of 4-[(2-chloro-4-nitro-phenoxy)methyl]-2-methyl-thiazole (600 mg, 2.11 mmol, 1 eq) in THF (10 mL) and H₂O (2 mL) was added NH₄Cl (1.13 g, 21.1 mmol, 10 eq) and copper; zinc (2.72 g, 21.1 mmol, 10 eq). The mixture was stirred at 25° C. for 5 hours. TLC (PE:EA=5:1, RF=0.3) indicated one major new spot was detected. LC-MS showed 95% of desired compound was detected. The reaction mixture was diluted with H₂O (50 mL) extracted with EA (20 mL*3). The combined organic layers were washed with brine (20 mL*3) and dried over Na₂SO₄, filtered and concentrated under reduced pressure to give 3-chloro-4-[(2-methylthiazol-4-yl)methoxy]aniline (500 mg, crude) as a yellow solid.

### Step 3: 1-(4-(4-((3-chloro-4-((2-methylthiazol-4-yl)methoxy)phenyl)amino)-7H-pyrrolo[2,3-d]pyrimidin-5-yl)piperidin-1-yl)prop-2-en-1-one

To a solution of 1-[4-(4-chloro-7H-pyrrolo[2,3-d]pyrimidin-5-yl)-1-piperidyl]prop-2-en-1-one (570 mg, 1.96 mmol, 1 eq), 3-chloro-4-[(2-methylthiazol-4-yl)methoxy]aniline (499 mg, 1.96 mmol, 1 eq) and TsOH (1.01 g, 5.88 mmol, 3 eq) in DMF (5 mL) and ACN (5 mL). The mixture was stirred at 70° C. for 16 hours. LC-MS showed little of 1-[4-(4-chloro-7H-pyrrolo[2,3-d]pyrimidin-5-yl)-1-piperidyl]prop-2-en-1-one remained. Several new peaks were shown on LC-MS and 58% of desired compound was detected. The reaction mixture was dropped into H₂O (200 mL) and the suspension was filtrated to get a yellow solid with 80% of desired mass. The crude product was purified by reversed-phase HPLC (column: Waters Xbridge C18 150*50 mm*10 μm; mobile phase: [water (NH₄HCOs)-ACN]; B %: 29%-59%, 10 min) to give 1-[4-[4-[3-chloro-4-[(2-methylthiazol-4-yl)methoxy]anilino]-7H-pyrrolo[2,3-d]pyrimidin-5-yl]-1-piperidyl]prop-2-en-1-one (340 mg, 659 μmol, 33.6% yield, 98.7% purity) as a white solid. ¹H NMR (400 MHz, DMSO-d₆) δ 11.59 (s, 1H), 8.18 (s, 1H), 7.98 (s, 1H), 7.81-7.80 (d, J=4 Hz, 1H), 7.56 (s, 1H), 7.54-7.53 (d, J=4 Hz, 1H), 7.29-7.27 (d, J=8 Hz, 1H), 7.04-7.03 (d, J=4 Hz, 1H), 6.82 (m, 1H), 6.12-6.11 (d, J=4 Hz, 1H), 5.68-5.67 (d, J=4 Hz, 1H), 5.20 (s, 1H), 4.54 (m, 1H), 4.51 (m, 1H), 3.51 (m, 1H), 3.27 (m, 1H), 2.83 (m, 1H), 2.67 (s, 3H), 2.03-2.01 (m, 2H), 1.45-1.41 (m, 2H).

### Example 22: Preparation of (R)-1-(4-(4-((3-chloro-4-(1-(thiazol-4-yl)ethoxy)phenyl)amino)-7H-pyrrolo[2,3-d]pyrimidin-5-yl)piperidin-1-yl)prop-2-en-1-one (Compound 218)

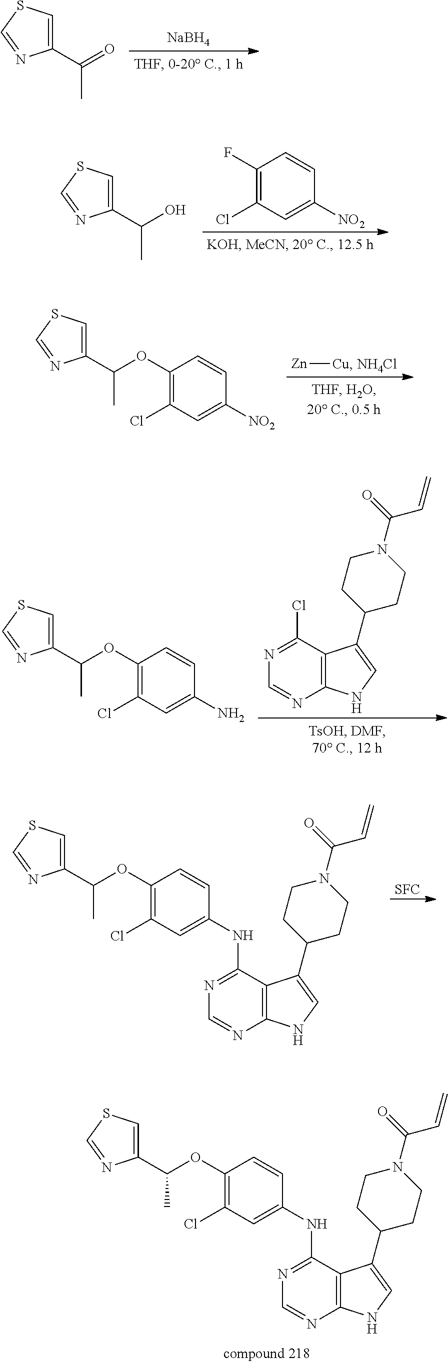

### Step 1: Preparation of 1-(thiazol-4-yl)ethan-1-ol

To a solution of 1-(thiazol-4-yl)ethan-1-one (2 g, 15.7 mmol, 1 eq) in THF (20 mL) was added NaBH₄ (1.76 g, 46.5 mmol, 2.96 eq) at 0° C. with portions over 30 minutes. Then the whole mixture was stirred at 20° C. for 0.5 hour. TLC (PE/EA=3:1) indicated none of 116-17d-1 was remained and one major new spot with larger polarity was detected. The mixture was quenched by saturation NH₄Cl solution (50 mL) and extracted with ethyl acetate (50 mL*2), dried over anhydrous sodium sulfate and concentrated under reduced pressure to give 1-(thiazol-4-yl)ethan-1-ol (1.8 g, crude) as a yellow oil. ¹H NMR (DMSO-d₆, 400 MHz) δ=9.01 (d, J=2.0 Hz, 1H), 7.45-7.39 (m, 1H), 5.31 (d, J=4.8 Hz, 1H), 4.90-4.81 (m, 1H), 1.40 (d, J=6.4 Hz, 3H).

### Step 2: Preparation of 4-(1-(2-chloro-4-nitrophenoxy)ethyl)thiazole

To a solution of 1-(thiazol-4-yl)ethan-1-ol (1.6 g, 12.4 mmol, 1 eq) in MeCN (20 mL) was added KOH (1.04 g, 18.6 mmol, 1.5 eq) and the mixture was stirred at 20° C. for 0.5 hour. Then 2-chloro-1-fluoro-4-nitrobenzene (2.17 g, 12.4 mmol, 1 eq) was added to the above mixture. The resulting mixture was stirred at 20° C. for 12 hours. TLC (PE:EA=3:1, Rf=0.31) indicated none of 116-17d-2 was remained, and one major new spot with lower polarity was detected. The mixture was poured into water (400 ml) and lots of yellow solid precipitated, filtered and the filter cake was concentrated under reduced pressure to give 4-(1-(2-chloro-4-nitrophenoxy)ethyl)thiazole (3.4 g, 11.7 mmol, 94.3% yield, 97.8% purity) as a yellow solid. ¹H NMR (DMSO-d₆, 400 MHz) δ=9.11 (d, J=2.0 Hz, 1H), 8.30 (d, J=2.8 Hz, 1H), 8.19-8.12 (m, 1H), 7.76 (d, J=1.6 Hz, 1H), 7.46 (d, J=9.6 Hz, 1H), 6.06-5.98 (m, 1H), 1.72 (d, J=6.4 Hz, 3H).

### Step 3: Preparation of 3-chloro-4-(1-(thiazol-4-yl)ethoxy)aniline

To a solution of 4-[1-(2-chloro-4-nitro-phenoxy)ethyl]thiazole (3.3 g, 11.6 mmol, 1 eq) in THF (30 mL) and H₂O (30 mL) was added copper; zinc (15.0 g, 116 mmol, 10 eq) and NH₄Cl (6.20 g, 116 mmol, 10 eq). The mixture was stirred at 20° C. for 0.5 hour. TLC (PE:EA=3:1, Rf=0.10) indicated no 116-17d-3 was remained and one major new spot with larger polarity was detected. The reaction mixture was filtered, and the filtrate was partitioned between water (50 mL) and EA (50 mL). The organic phase was separated and washed with water (50 mL*2), concentrated under reduced pressure to give 3-chloro-4-(1-(thiazol-4-yl)ethoxy)aniline (3 g, 11.4 mmol, 98.6% yield, 97% purity) as a yellow liquid. ¹H NMR (DMSO-d₆, 400 MHz) δ=9.07 (d, J=2.0 Hz, 1H), 7.58 (d, J=1.6 Hz, 1H), 6.75 (d, J=8.8 Hz, 1H), 6.60 (d, J=2.8 Hz, 1H), 6.41-6.35 (m, 1H), 5.44-5.33 (m, 1H), 4.95 (s, 2H), 1.59 (d, J=6.4 Hz, 3H).

### Step 4: Preparation of 1-(4-(4-((3-chloro-4-(1-(thiazol-4-yl)ethoxy)phenyl)amino)-7H-pyrrolo[2,3-d]pyrimidin-5-yl)piperidin-1-yl)prop-2-en-1-one

To a solution of 3-chloro-4-(1-thiazol-4-ylethoxy)aniline (1 g, 3.93 mmol, 1 eq) and 1-(4-(4-chloro-7H-pyrrolo[2,3-d]pyrimidin-5-yl)piperidin-1-yl)prop-2-en-1-one (1.14 g, 3.93 mmol, 1 eq) in DMF (20 mL) was added TsOH (1.35 g, 7.85 mmol, 2 eq). The mixture was stirred at 70° C. for 12 hours. TLC (PE:EA=0:1, Rf=0.12) indicated ˜20% of 116-17d-4 was remained, and one major new spot with larger polarity was detected. The reaction mixture was partitioned between NaHCO₃ (50 mL, aq) and EA (50 mL). The organic phase was separated and washed with NaHCO₃ (50 mL*2), concentrated under reduced pressure to give a residue. The residue was purified by column chromatography (SiO₂, CH₂Cl₂/MeOH=10/1 to 9/1) to give 1 g yellow solid and the yellow solid was purified by prep-HPLC (column: Phenomenex luna C18 150*40 mm*15 μm; mobile phase: [water (FA)-ACN]; B %: 22%-52%, 15 min) to give 1-(4-(4-((3-chloro-4-(1-(thiazol-4-yl)ethoxy)phenyl)amino)-7H-pyrrolo[2,3-d]pyrimidin-5-yl)piperidin-1-yl)prop-2-en-1-one (500 mg, 932 μmol, 23.8% yield, 94.9% purity) as a white solid. ¹H NMR (DMSO-d₆, 400 MHz) δ=11.59 (br d, J=1.6 Hz, 1H), 9.10 (d, J=2.0 Hz, 1H), 8.17 (s, 1H), 7.95 (s, 1H), 7.80 (d, J=2.8 Hz, 1H), 7.67 (d, J=1.6 Hz, 1H), 7.48-7.43 (m, 1H), 7.15 (d, J=9.2 Hz, 1H), 7.03 (d, J=2.0 Hz, 1H), 6.87-6.77 (m, 1H), 6.15-6.06 (m, 1H), 5.74-5.63 (m, 2H), 4.55 (br d, J=12.4 Hz, 1H), 4.17-4.09 (m, 1H), 3.54-3.44 (m, 1H), 3.42-3.36 (m, 1H), 3.27-3.20 (m, 1H), 2.82 (br t, J=12.0 Hz, 1H), 2.08-1.99 (m, 2H), 1.67 (d, J=6.4 Hz, 3H), 1.50-1.37 (m, 2H)

### Step 5: Preparation of (R)-1-(4-(4-((3-chloro-4-(1-(thiazol-4-yl)ethoxy)phenyl)amino)-7H-pyrrolo[2,3-d]pyrimidin-5-yl)piperidin-1-yl)prop-2-en-1-one

The compound 1-[4-[4-[3-chloro-4-(1-thiazol-4-ylethoxy)anilino]-7H-pyrrolo[2,3-d]pyrimidin-5-yl]-1-piperidyl]prop-2-en-1-one (500 mg, 982 μmol, 1 eq) was dissolved in MeOH (20 mL) and purified by prep-SFC (column: REGIS(S,S)WHELK-O1 (250 mm*25 mm, 10 μm); mobile phase: [IPA-ACN]; B %: 45%-45%, 5.6 min) to give (R)-1-(4-(4-((3-chloro-4-(1-(thiazol-4-yl)ethoxy)phenyl)amino)-7H-pyrrolo[2,3-d]pyrimidin-5-yl)piperidin-1-yl)prop-2-en-1-one (250 mg, 491 μmol, 50.0% yield) as a white solid. ¹H NMR (CDCl₃, 400 MHz) δ=10.09 (br s, 1H), 8.82 (d, J=2.0 Hz, 1H), 8.41 (s, 1H), 7.78 (d, J=2.8 Hz, 1H), 7.38 (d, J=2.0 Hz, 1H), 7.36-7.32 (m, 1H), 6.97-6.87 (m, 2H), 6.69-6.58 (m, 2H), 6.37-6.27 (m, 1H), 5.76-5.70 (m, 1H), 5.67-5.59 (m, 1H), 4.95-4.76 (m, 1H), 4.29-4.10 (m, 1H), 3.36-3.14 (m, 1H), 3.05-2.94 (m, 1H), 2.90-2.74 (m, 1H), 2.19 (br d, J=13.2 Hz, 2H), 1.79 (d, J=6.4 Hz, 3H), 1.75-1.67 (m, 2H)

### Example 23: Preparation of 1-(4-(4-((3-chloro-4-((5-methylthiazol-4-yl)methoxy)phenyl)amino)-7H-pyrrolo[2,3-d]pyrimidin-5-yl)piperidin-1-yl)prop-2-en-1-one (Compound 219)

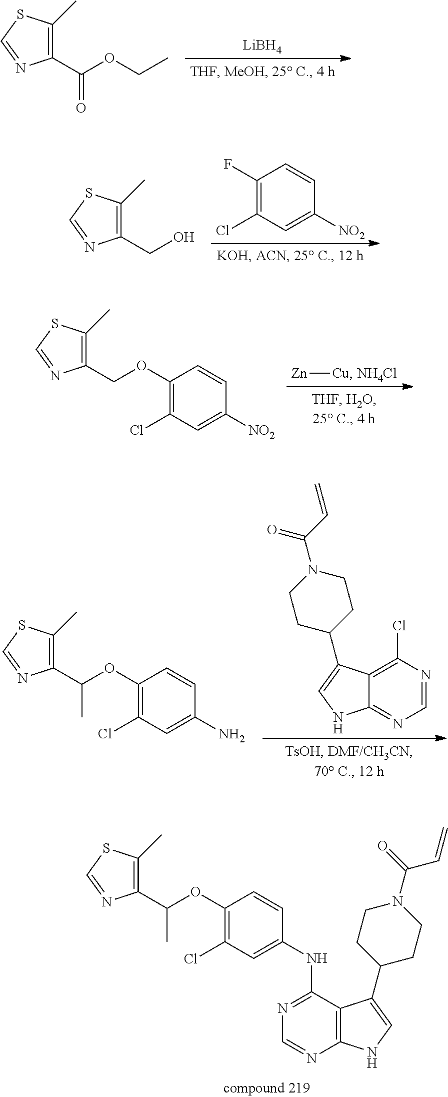

### Step 1: Preparation of (5-methylthiazol-4-yl)methanol

To a mixture of ethyl 5-methylthiazole-4-carboxylate (2 g, 11.7 mmol, 1 eq) in dry THF (40 mL) under N₂ was added LiBH₄ (509 mg, 23.4 mmol, 23.4 mL, 2 eq) at 0° C., followed by MeOH (749 mg, 23.4 mmol, 2 eq) with dropwise. The mixture was stirred at 25° C. for 4 hours. TLC (PE:EA=1:1, RF=0.2) indicated the starting material was consumed and a new spot was detected. The reaction mixture was quenched by addition saturated aqueous NH₄Cl (20 mL) at 0° C. and then extracted with EA (20 mL*3). The combined organic layers were washed with brine (30 mL*2), dried over by Na₂SO₄, filtered and concentrated under reduced pressure to give a residue. The residue was purified by column chromatography (SiO₂, Petroleum ether/Ethyl acetate=1/0 to 1/1) to give (5-methylthiazol-4-yl)methanol (0.42 g, 3.25 mmol, 27.8% yield) as a colorless oil. ¹H NMR (400 MHz, DMSO-d₆) δ=8.78 (s, 1H), 5.03 (s, 1H), 4.51 (s, 2H), 2.44 (s, 3H).

### Step 2: Preparation of 4-((2-chloro-4-nitrophenoxy)methyl)-5-methythiazole

To a solution of (5-methylthiazol-4-yl) methanol (420 mg, 3.25 mmol, 1 eq) in MeCN (10 mL) was added KOH (547 mg, 9.75 mmol, 3 eq) and 2-chloro-1-fluoro-4-nitro-benzene (571 mg, 3.25 mmol, 1 eq). The mixture was stirred at 25° C. for 12 hours. LCMS showed 88% desired mass was detected. The reaction mixture was poured into H₂O (200 mL) and stirred for 10 minutes, then filtered to give 4-((2-chloro-4-nitrophenoxy)methyl)-5-methylthiazole (800 mg, 2.81 mmol, 86.4% yield) as a yellow solid. ¹H NMR (400 MHz, DMSO-d₆) δ=8.90 (s, 1H), 8.30 (s, 1H), 8.24 (d, J=8.8 Hz, 1H), 7.56 (d, J=8.8 Hz, 1H), 5.42 (s, 2H), 2.52 (s, 3H).

### Step 3: Preparation of 3-chloro-4-((5-methylthiazol-4-yl)methoxy)aniline

To a solution of 4-[(2-chloro-4-nitro-phenoxy) methyl]-5-methyl-thiazole (700 mg, 2.46 mmol, 1 eq) and NH₄Cl (658 mg, 12.3 mmol, 5 eq) in THF (20 mL) H₂O (4 mL) was added copper; zinc (1.58 g, 12.3 mmol, 5 eq). The reaction mixture was stirred at 25° C. for 4 hours. LCMS showed 96% desired mass was detected. The reaction mixture was filtered, and the liquid was diluted with EA (30 mL), and then filtered again. The organic layer was washed with brine (10 mL*3), dried over by Na₂SO₄, filtered and concentrated under reduced pressure to give 3-chloro-4-((5-methylthiazol-4-yl)methoxy)aniline (600 mg, 2.36 mmol, 95.80% yield) as a yellow solid. ¹H NMR (400 MHz, DMSO-d₆) δ=8.85 (s, 1H), 6.92 (d, J=8.6 Hz, 1H), 6.62 (d, J=2.8 Hz, 1H), 6.51-6.39 (m, 1H), 5.02 (s, 2H), 4.95 (s, 2H), 2.44 (s, 3H).

### Step 4: Preparation of 1-(4-(4-((3-chloro-4-((5-methylthiazol-4-yl)methoxy)phenyl)amino)-7H-pyrrolo[2,3-d]pyrimidin-5-yl)piperidin-1-yl)prop-2-en-1-one

A solution of 3-chloro-4-[(5-methylthiazol-4-yl) methoxy] aniline (500 mg, 1.96 mmol, 1 eq) 1-[4-(4-chloro-7H-pyrrolo[2,3-d] pyrimidin-5-yl)-1-piperidyl] prop-2-en-1-one (570 mg, 1.96 mmol, 1 eq) TsOH (1.01 g, 5.89 mmol, 3 eq) in DMF (6 mL) and MeCN (6 mL) was stirred at 70° C. for 12 hours. LCMS showed 60% desired mass was detected. The reaction mixture was diluted with NaHCO₃ saturated aqueous solution (200 mL) and extracted with EA (50 mL*3). The combined organic layers were washed with brine (100 mL*3), dried over Na₂SO₄, filtered, and concentrated under reduced pressure to give a residue. The residue was first purified by column chromatography (SiO₂, Petroleum ether/Ethyl acetate=1/1 to 0/1). The residue was purified by prep-HPLC (column: Phenomenex Luna C18 150*25 mm*10 μm; mobile phase: [water(FA)-ACN]; B %: 21%-51%, 10 min) to give 1-(4-(4-((3-chloro-4-((5-methylthiazol-4-yl)methoxy)phenyl)amino)-7H-pyrrolo[2,3-d]pyrimidin-5-yl)piperidin-1-yl)prop-2-en-1-one (270 mg, 524 μmol, 26.7% yield, 98.7% purity) as a white solid.

¹H NMR (400 MHz, DMSO-d₆) δ=11.61 (s, 1H), 8.90 (s, 1H), 8.18 (s, 1H), 7.99 (s, 1H), 7.80 (d, J=2.8 Hz, 1H), 7.58-7.51 (m, 1H), 7.30 (d, J=9.2 Hz, 1H), 7.04 (d, J=2.0 Hz, 1H), 6.88-6.78 (m, 1H), 6.15-6.06 (m, 1H), 5.70-5.63 (m, 1H), 5.24 (s, 2H), 4.56 (d, J=12.4 Hz, 1H), 4.14 (d, J=13.2 Hz, 1H), 3.57-3.49 (m, 1H), 3.27 (br s, 1H), 2.90-2.78 (m, 1H), 2.52 (s, 3H), 2.03 (s, 2H), 1.50-1.37 (m, 2H).

### Example 24: Preparation of 1-[4-[4-[3-bromo-2-fluoro-4-(2-pyridylmethoxy)anilino]-7H-pyrrolo[2,3-d]pyrimidin-5-yl]-1-piperidyl]prop-2-en-1-on (Compound 220)

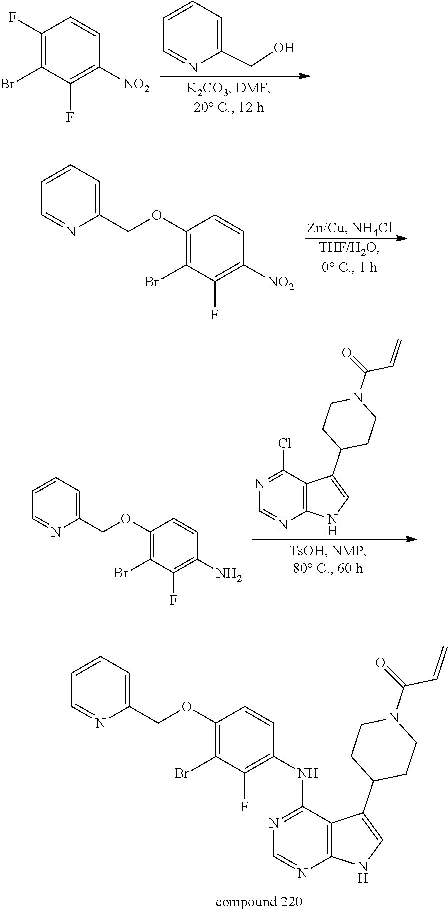 [SMILES: O=[N+]([O-])c1ccc(F)c(Br)c1F ; OCc1ccccn1 ; O=[N+]([O-])c1ccc(OCc2ccccn2)c(Br)c1F ; Nc1ccc(OCc2ccccn2)c(Br)c1F ; C=CC(=O)N1CCC(c2c[nH]c3ncnc(Cl)c23)CC1 ; C=CC(=O)N1CCC(c2c[nH]c3ncnc(Nc4ccc(OCc5ccccn5)c(Br)c4F)c23)CC1]

### Step 1: 2-[(2-bromo-3-fluoro-4-nitro-phenoxy)methyl]pyridine

To a solution of 2-bromo-1,3-difluoro-4-nitro-benzene (5 g, 21.0 mmol, 1 eq) and 2-pyridylmethanol (2.29 g, 21.01 mmol, 1 eq) in DMF (50 mL, N/A purity) was added K₂CO₃ (5.81 g, 42.0 mmol, 2 eq). The mixture was stirred at 20° C. for 12 hours. LCMS showed desired mass was formed. TLC (PE/EA-3/1) showed 134-57b-1 remained and several new spots were formed. The mixture was poured into ice water (600 mL) and filtered to yield a filter cake. The cake was filtered and the cake was concentrated to give compound 2-[(2-bromo-3-fluoro-4-nitro-phenoxy)methyl]pyridine (2.3 g, 6.40 mmol, 30.5% yield, 91% purity) as white solid. ¹H NMR (400 MHz, DMSO-d₆) δ=8.60 (d, J=4.4 Hz, 1H), 8.25 (t, J=9.2 Hz, 1H), 7.88-7.92 (m, 1H), 7.59 (d, J=7.6 Hz, 1H), 7.38-7.40 (m, 1H), 7.28-7.30 (m, 1H), 5.48 (s, 2H).

### Step 2: 3-bromo-2-fluoro-4-(2-pyridylmethoxy)aniline

To a solution of 2-[(2-bromo-3-fluoro-4-nitro-phenoxy)methyl]pyridine (1 g, 2.78 mmol, 91% purity, 1 eq), NH₄Cl (744.06 mg, 13.9 mmol, 5 eq) in THF (30 mL) and H₂O (6 mL) was added copper; zinc (1.79 g, 13.9 mmol, 5 eq) at 0° C. The mixture was stirred at 0° C. for 1 hour. LCMS showed 134-57b-2 was consumed and desired mass was formed. The mixture was filtered. The filtrate was added H₂O (100 mL), extracted with EA (50 mL×2), the combined organic layer was washed with brine (100 mL×1), dried over Na₂SO₄, filtered and concentrated to give a residue, which purified by prep-HPLC (neutral, column: Waters Xbridge C18 150*50 mm*10 μm; mobile phase: [water(NH₄HCOs)-ACN]; B %: 27%-57%, 10 min), lyophilized to give compound 3-bromo-2-fluoro-4-(2-pyridylmethoxy)aniline (0.42 g, 1.41 mmol, 50.81% yield) as yellow solid. ¹H NMR (400 MHz, DMSO-d₆) δ=8.55-8.57 (m, 1H), 7.84-7.86 (m, 1H), 7.56 (d, J=7.6 Hz, 1H), 7.35-7.32 (m, 1H), 6.83-6.68 (m, 2H), 5.12 (s, 2H), 4.96 (s, 2H).

### Step 3: 1-[4-[4-[3-bromo-2-fluoro-4-(2-pyridylmethoxy)anilino]-7H-pyrrolo[2,3-d]pyrimidin-5-yl]-1-piperidyl]prop-2-en-1-one

To a solution of 3-bromo-2-fluoro-4-(2-pyridylmethoxy)aniline (215 mg, 724 μmol, 1 eq) and 1-[4-(4-chloro-7H-pyrrolo[2,3-d]pyrimidin-5-yl)-1-piperidyl]prop-2-en-1-one (210 mg, 72 μmol, 1 eq) in NMP (20 mL) was added TsOH (374 mg, 2.17 mmol, 3 eq). The mixture was stirred at 80° C. for 60 hours. LCMS showed 134-57b-3 remained and desired mass was formed. The mixture was poured into saturated aqueous NaHCO₃ (200 mL), the mixture was extracted with EA (100 mL×3), the combined organic layer was washed with brine (200 mL×1), dried over Na₂SO₄, filtered and concentrated to give a crude. The crude was purified by silica column (PE/EA=5/1 to 0/1, EA/MeOH-50/1 to 10/1) to give a residue, which was purified by prep-HPLC (FA, column: Phenomenex luna C18 150*25 mm*10 μm; mobile phase: [water(FA)-ACN]; B %: 24%-54%, min), lyophilized to give compound 1-[4-[4-[3-bromo-2-fluoro-4-(2-pyridylmethoxy)anilino]-7H-pyrrolo[2,3-d]pyrimidin-5-yl]-1-piperidyl]prop-2-en-1-one (170 mg, 302 μmol, 20.8% yield, 97.8% purity) as yellow solid. ¹H NMR (400 MHz, DMSO-d₆) δ=11.57 (s, 1H), 8.61 (d, J=4.0 Hz, 1H), 8.07 (s, 1H), 7.95 (s, 1H), 7.89 (t, J=7.2 Hz, 1H), 7.63-7.54 (m, 2H), 7.40-7.35 (m, 1H), 7.09 (d, J=8.8 Hz, 1H), 7.03 (s, 1H), 6.88-6.80 m, 1H), 6.17-6.04 (m, 1H), 5.70-5.60 (m, 1H), 5.35 (s, 2H), 4.58-4.56 (m, 1H), 4.17-4.15 (m, 1H), 3.49-3.37 (m, 1H), 3.29-3.20 (m, 1H), 2.83-2.81 (m, 1H), 2.12-2.00 (m, 2H), 1.46-1.44 (m, 2H).

### Example 25: Preparation of 1-(4-(4-((5-chloro-2-fluoro-4-(pyridin-2-ylmethoxy)phenyl)amino)-7H-pyrrolo[2,3-d]pyrimidin-5-yl)piperidin-1-yl)prop-2-en-1-one (Compound 221)

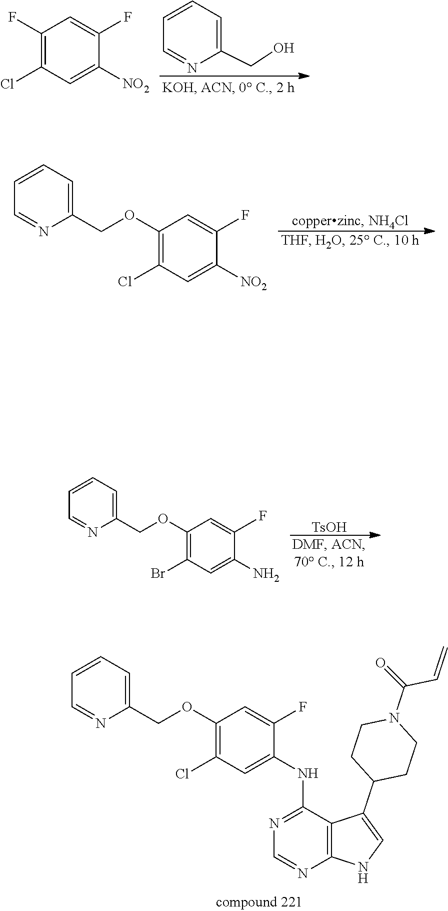

### Step 1: Preparation of 2-((2-chloro-5-fluoro-4-nitrophenoxy)methyl)pyridine

To a solution of 1-chloro-2,4-difluoro-5-nitro-benzene (9.4 g, 48.6 mmol, 1 eq) in ACN (150 mL) was added KOH (8.18 g, 146 mmol, 3 eq) and 2-pyridylmethanol (5.30 g, 48.6 mmol, 4.69 mL, 1 eq) at 0° C. The mixture was stirred at 0° C. for 2 hours. LC-MS showed no 1-chloro-2,4-difluoro-5-nitro-benzene remained. Several new peaks were shown on LC-MS and the main desired compound was detected. The mixture was added into water (100 mL), then suspension was filtered, the filter cake is vacuum-dried to obtain the coarse product to give 2-((2-chloro-5-fluoro-4-nitrophenoxy)methyl)pyridine (12 g, 42.5 mmol, 87.4% yield)) as a yellow solid. 1H NMR (400 MHz, DMSO-d₆) δ=8.65-8.56 (m, 1H), 8.37-8.30 (m, 1H), 7.91 (br d, J=1.8 Hz, 1H), 7.65-7.60 (m, 1H), 7.59-7.54 (m, 1H), 7.44-7.35 (m, 1H), 5.72-5.11 (m, 2H)

### Step 2: Preparation of 5-chloro-2-fluoro-4-(pyridin-2-ylmethoxy)aniline

To a solution of 2-[(2-chloro-5-fluoro-4-nitro-phenoxy)methyl]pyridine (11 g, 38.9 mmol, 1 eq) in THF (150 mL) and H₂O (30 mL) was added copper; zinc (25.1 g, 195 mmol, 5 eq) and NH₄Cl (10.4 g, 195 mmol, 5 eq). The mixture was stirred at 25° C. for 10 hours. LC-MS showed no 2-[(2-chloro-5-fluoro-4-nitro-phenoxy)methyl]pyridine remained. Several new peaks were shown on LC-MS and 44% of desired compound was detected. The mixture was filtrated, and the filtrate was extracted with ethyl acetate (100 mL*3). The combined organic phase was washed with brine (50 mL*3), dried with anhydrous Na₂SO₄, filtered, and concentrated in vacuum to give a residue. The residue (PE:EA=3:1, R_{f}=0.3) was purified by column chromatography (SiO₂, Petroleum ether/Ethyl acetate=1/0 to 3/1, Rf=0.3) to give 5-chloro-2-fluoro-4-(pyridin-2-ylmethoxy)aniline (5 g, 19.8 mmol, 50.9% yield) as a yellow solid. 1H NMR (400 MHz, DMSO-d₆) δ=8.67-8.49 (m, 1H), 7.98-7.75 (m, 1H), 7.56 (s, 1H), 7.41-7.31 (m, 1H), 7.07 (d, J=12.6 Hz, 1H), 6.87 (d, J=9.4 Hz, 1H), 5.12 (s, 2H), 4.96 (s, 2H)

### Step 3: Preparation of 1-(4-(4-((5-chloro-2-fluoro-4-(pyridin-2-ylmethoxy)phenyl)amino)-7H-pyrrolo[2,3-d]pyrimidin-5-yl)piperidin-1-yl)prop-2-en-1-one

To a solution of 5-chloro-2-fluoro-4-(2-pyridylmethoxy)aniline (1 g, 3.96 mmol, 1 eq) and 1-[4-(4-chloro-7H-pyrrolo[2,3-d]pyrimidin-5-yl)-1-piperidyl]prop-2-en-1-one (1.15 g, 3.96 mmol, 1 eq) in DMF (10 mL) and ACN (10 mL) was added TsOH (2.04 g, 11.9 mmol, 3 eq). The mixture was stirred at 70° C. for 12 hours. LC-MS showed no 5-chloro-2-fluoro-4-(2-pyridylmethoxy)aniline remained. Several new peaks were shown on LC-MS and 65% of desired compound was detected. The mixture was added NaHCO₃ to adjust pH=7. Then the reaction mixture was diluted with water (30 mL) and extracted with EA (15 mL*3). The combined organic layers were washed with brine (15 mL*2), dried over Na₂SO₄, filtered, and concentrated under reduced pressure to give a residue. The residue was purified by prep-HPLC (neutral condition; column: Waters X bridge C18 150*50 mm*10 μm; mobile phase: [water(NH₄HCO₃)-ACN]; B %: 29%-59%, 10 min). The residue was purified by prep-HPLC (TFA condition, column: YMCTriart C18150′25 mm*5 um; mobile phase: [water(TFA)-ACN]; B %: 30%-50%, 10 min) to give 1-(4-(4-((5-chloro-2-fluoro-4-(pyridin-2-ylmethoxy)phenyl)amino)-7H-pyrrolo[2,3-d]pyrimidin-5-yl)piperidin-1-yl)prop-2-en-1-one (119 mg, 230 μmol, 5.81% yield, 98% purity) as a white solid.

1H NMR (400 MHz, DMSO-d₆) δ=11.59 (s, 1H), 8.69-8.53 (m, 1H), 8.11 (s, 1H), 7.96-7.78 (m, 3H), 7.59 (d, J=8.0 Hz, 1H), 7.41-7.30 (m, 2H), 7.04 (d, J=1.8 Hz, 1H), 6.83 (s, 1H), 6.18-6.03 (m, 1H), 5.74-5.57 (m, 1H), 5.33 (s, 2H), 4.66-4.46 (m, 1H), 4.26-4.08 (m, 1H), 3.45-3.39 (m, 1H), 3.28-3.21 (m, 1H), 2.87-2.76 (m, 1H), 2.12-2.01 (m, 2H), 1.54-1.38 (m, 2H).

### Example 26: Preparation of 1-[4-[4-[2,5-dichloro-4-(2-pyridylmethoxy)anilino]-7H-pyrrolo[2,3-d]pyrimidin-5-yl]-1-piperidyl]prop-2-en-1-one (Compound 222)

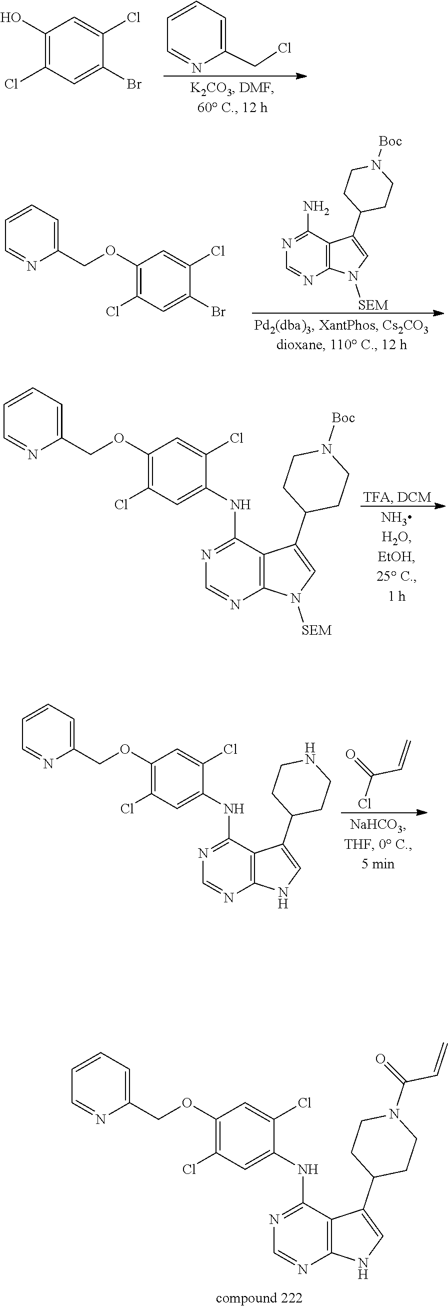

### Step 1: Preparation of 2-[(4-bromo-2,5-dichloro-phenoxy)methyl]pyridine

To a solution of 4-bromo-2,5-dichloro-phenol (5 g, 20.7 mmol, 1 eq) in DMF (50 mL) was added K₂CO₃ (14.3 g, 103 mmol, 5 eq) and 2-(chloromethyl)pyridine (2.77 g, 21.7 mmol, 1.05 eq). The mixture was stirred at 60° C. for 12 hours. LC-MS showed 4-bromo-2,5-dichloro-phenol was consumed completely and 86% of desired compound was detected. The reaction mixture was quenched by addition NaHCO₃ (100 mL) at 0° C., solids separated out and concentrated under reduced pressure to give 2-[(4-bromo-2,5-dichloro-phenoxy)methyl]pyridine (5.3 g, 15.92 mmol, 77% yield) as a brown solid. 1H NMR (DMSO-d₆, 400 MHz) δ=8.59 (br d, J=4.4 Hz, 1H), 7.92 (s, 2H), 7.68-7.50 (m, 2H), 7.37 (dd, J=5.2, 6.4 Hz, 1H), 5.32 (s, 2H).

### Step 2: Preparation of 4-[4-[2,5-dichloro-4-(2-pyridylmethoxy)anilino]-7-(2-trimethylsilylethoxymethyl)pyrrolo[2,3-d]pyrimidin-5-yl]piperidine-1-carboxylate

A mixture of 2-[(4-bromo-2,5-dichloro-phenoxy)methyl]pyridine (500 mg, 1.50 mmol, 1 eq), tert-butyl 4-[4-amino-7-(2-trimethylsilylethoxymethyl)pyrrolo[2,3-d]pyrimidin-5-yl]piperidine-1-carboxylate (672 mg, 1.50 mmol, 1 eq), Cs₂CO₃ (1.47 g, 4.50 mmol, 3 eq), (5-diphenylphosphanyl-9,9-dimethyl-xanthen-4-yl)-diphenyl-phosphane (348 mg, 601 μmol, 0.4 eq) and (1E,4E)-1,5-diphenylpenta-1,4-dien-3-one; palladium (275 mg, 300 μmol, 0.2 eq) in dioxane (15 mL) was degassed and purged with N₂ for 3 times and then the mixture was stirred at 110° C. for 12 hours under N₂ atmosphere. LC-MS showed 2-[(4-bromo-2,5-dichloro-phenoxy)methyl]pyridine was consumed completely, and 70% of desired compound was detected. The reaction mixture was quenched by addition NH₄Cl (100 mL) at 0° C. and extracted with EA (20 mL*2). The combined organic layers were washed with NaCl (20 mL*2), dried over Na₂SO₄, filtered, and concentrated under reduced pressure to give a residue. Then concentrated under reduced pressure to give a residue. The residue was purified by column chromatography (SiO₂, Petroleum ether/Ethyl acetate-8/1 to 3/1) to give 4-[4-[2,5-dichloro-4-(2-pyridylmethoxy)anilino]-7-(2-trimethylsilylethoxymethyl)pyrrolo[2,3-d]pyrimidin-5-yl]piperidine-1-carboxylate (1.3 g, 1.86 mmol, 88% yield) as a brown oil. 1H NMR (DMSO-d₆, 400 MHz) δ=8.70-8.51 (m, 1H), 8.22 (s, 1H), 8.08 (s, 1H), 7.95 (s, 1H), 7.92-7.85 (m, 1H), 7.59 (d, J=7.6 Hz, 1H), 7.50 (s, 1H), 7.38 (dd, J=4.8, 6.4 Hz, 1H), 7.26 (s, 1H), 5.49 (s, 2H), 5.36 (s, 2H), 4.16-3.93 (m, 2H), 3.51-3.48 (m, 2H), 3.31-3.25 (m, 1H), 3.08-2.80 (m, 2H), 2.11-1.99 (m, 2H), 1.55-1.28 (m, 11H), 0.82-0.79 (m, 2H), 0.03-0.24 (m, 9H).

### Step 3: Preparation of N-[2,5-dichloro-4-(2-pyridylmethoxy)phenyl]-5-(4-piperidyl)-7H-pyrrolo[2,3-d]pyrimidin-4-amine

To a solution of tert-butyl4-[4-[2,5-dichloro-4-(2-pyridylmethoxy)anilino]-7-(2-trimethylsilylethoxymethyl)pyrrolo[2,3-d]pyrimidin-5-yl]piperidine-1-carboxylate (1.3 g, 1.86 mmol, 1 eq) in TFA (10 mL) and DCM (10 mL). The mixture was stirred at 25° C. for 1 hour. LC-MS showed tert-butyl4-[4-[2,5-dichloro-4-(2-pyridylmethoxy)anilino]-7-(2-trimethylsilylethoxymethyl)pyrrolo[2,3-d]pyrimidin-5-yl]piperidine-1-carboxylate was consumed completely and 80% of desired compound was detected. The reaction mixture was quenched by addition NaHCO₃ (100 mL) at 0° C. The residue was extracted with EA (20 mL*2). The combined organic layers were washed with NaCl (30 mL*2), dried over Na₂SO₄, filtered, and concentrated under reduced pressure to give a residue. The crude product was triturated with MTBE (3 mL) at 25° C. for 30 minutes to give N-[2,5-dichloro-4-(2-pyridylmethoxy)phenyl]-5-(4-piperidyl)-7H-pyrrolo[2,3-d]pyrimidin-4-amine (700 mg, 1.49 mmol, 80% yield) as a white solid. 1H NMR (DMSO-d₆, 400 MHz) δ=11.66 (br d, J=3.2 Hz, 1H), 8.60 (d, J=4.8 Hz, 1H), 8.48-8.37 (m, 1H), 8.21 (s, 1H), 7.93-7.85 (m, 1H), 7.77-7.66 (m, 1H), 7.59 (d, J=7.6 Hz, 1H), 7.50 (d, J=1.6 Hz, 1H), 7.38 (dd, J=5.4, 7.2 Hz, 1H), 7.14-7.00 (m, 1H), 5.33 (s, 2H), 3.09 (br d, J=11.2 Hz, 1H), 3.06-2.91 (m, 2H), 2.70 (br s, 1H), 2.59 (br s, 1H), 2.20-2.01 (m, 2H), 1.71-1.44 (m, 2H).

### Step 4: Preparation of 1-[4-[4-[2,5-dichloro-4-(2-pyridylmethoxy)anilino]-7H-pyrrolo[2,3-d]pyrimidin-5-yl]-1-piperidyl]prop-2-en-1-one

To a solution of N-[2,5-dichloro-4-(2-pyridylmethoxy)phenyl]-5-(4-piperidyl)-7H-pyrrolo[2,3-d]pyrimidin-4-amine (700 mg, 1.49 mmol, 1 eq) in THF (4 mL) and NaHCO₃ (4 mL) was added prop-2-enoyl chloride (135 mg, 1.49 mmol, 1 eq) at 0° C. The mixture was stirred at 0° C. for 5 minutes. LC-MS showed N-[2,5-dichloro-4-(2-pyridylmethoxy)phenyl]-5-(4-piperidyl)-7H-pyrrolo[2,3-d]pyrimidin-4-amine was consumed completely and 67% of desired compound was detected. The reaction mixture was quenched by addition NH₄Cl 100 mL at 0° C., solid separated out and filtered, then concentrated under reduced pressure to give a residue. The residue was purified by normal prep-HPLC (column: Welch Ultimate XB—SiOH 250*50*10 um; mobile phase: [Hexane-EtOH]; B %: 5%-45%, 15 min) to give 1-[4-[4-[2,5-dichloro-4-(2-pyridylmethoxy)anilino]-7H-pyrrolo[2,3-d]pyrimidin-5-yl]-1-piperidyl]prop-2-en-1-one (145 mg, 255 μmol, 17% yield, 92% purity) as a white solid. 1H NMR (DMSO-d₆, 400 MHz) δ=11.64 (br s, 1H), 8.61 (d, J=4.8 Hz, 1H), 8.17 (d, J=5.6 Hz, 2H), 7.91-7.86 (m, 1H), 7.84 (s, 1H), 7.59 (d, J=7.6 Hz, 1H), 7.50 (s, 1H), 7.42-7.34 (m, 1H), 7.07 (s, 1H), 6.84 (dd, J=10.4, 16.8 Hz, 1H), 6.10 (dd, J=2.0, 16.8 Hz, 1H), 5.67 (dd, J=2.4, 10.4 Hz, 1H), 5.35 (s, 2H), 4.57 (br d, J=13.2 Hz, 1H), 4.24-4.11 (m, 1H), 3.28-3.19 (m, 2H), 2.87-2.75 (m, 1H), 2.15-2.06 (m, 2H), 1.54-1.42 (m, 2H).

### Example 27: Preparation of 1-[4-[4-[3-chloro-4-[(5-fluorothiazol-2-yl)methoxy]anilino]-7H-pyrrolo[2,3-d]pyrimidin-5-yl]-1-piperidyl]prop-2-en-1-one (Compound 223)

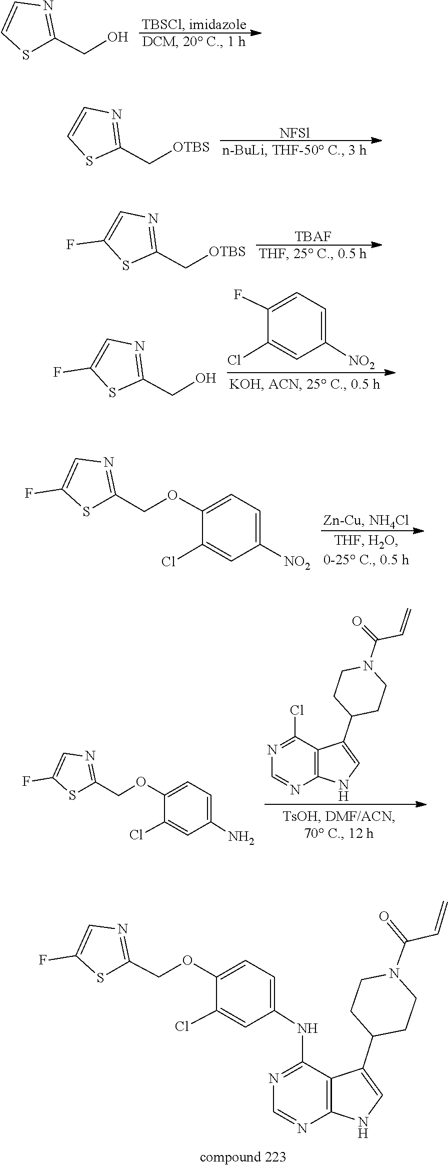

### Step 1: Preparation of tert-butyl-dimethyl-(thiazol-2-ylmethoxy)silane

To a solution of thiazol-2-ylmethanol (10 g, 86.8 mmol, 1 eq) in DCM (100 mL) was added imidazole (7.69 g, 112.9 mmol, 1.3 eq) and TBSCl (14 g, 92.9 mmol, 11.4 mL, 1.07 eq). The mixture was stirred at 20° C. for 1 hour. TLC (PE:EA=5:1) indicated thiazol-2-ylmethanol was consumed completely and one new spot formed. The reaction mixture was quenched by addition water (100 mL) at 20° C., and then diluted with DCM (10 mL) and extracted with DCM (100 mL*2). The combined organic layers were washed with water (100 mL*2), dried over Na₂SO₄, filtered and concentrated under reduced pressure to give a residue. The residue was purified by column chromatography (SiO₂, Petroleum ether/Ethyl acetate-1000/1 to 20/1) to give tert-butyl-dimethyl-(thiazol-2-ylmethoxy)silane (19 g, 82.8 mmol, 95% yield) as a yellow liquid. ¹H NMR (CDCl₃, 400 MHz) δ=7.73 (d, J=3.2 Hz, 1H), 7.28 (d, J=3.2 Hz, 1H), 5.00 (s, 2H), 0.97 (s, 8H), 0.15 (s, 6H).

### Step 2: Preparation of tert-butyl-[(5-fluorothiazol-2-yl)methoxy]-dimethy-slilane

To a solution of tert-butyl-dimethyl-(thiazol-2-ylmethoxy)silane (19 g, 82.8 mmol, 1 eq) in THF (300 mL) was added dropwise t-BuLi (1.3 M, 127 mL, 2 eq) at −50° C. over 1 hour. After addition, the mixture was stirred at this temperature for 1 hour and then N-(benzenesulfonyl)-N-fluoro-benzenesulfonamide (31.3 g, 99.4 mmol, 1.2 eq) in THF (50 mL) was added dropwise at −50° C. The resulting mixture was stirred at −50° C. for 1 hour. LC-MS showed 24.2% of tert-butyl-dimethyl-(thiazol-2-ylmethoxy)silane remained. Several new peaks were shown on LC-MS and 6.9% of desired compound was detected. TLC (PE:EA=20:1) indicated tert-butyl-dimethyl-(thiazol-2-ylmethoxy)silane was remained and many new spot were detected. The reaction mixture was quenched by the addition of saturated NH₄Cl (400 mL) at 0° C. slowly, and then diluted with EA (200 mL) and extracted with EA (300 mL*2). The combined organic layers were washed with water (300 mL*2), dried over Na₂SO₄, filtered, and concentrated under reduced pressure to give a residue. The residue was purified by column chromatography (SiO₂, Petroleum ether/Ethyl acetate=1/0 to 1000/1) to give tert-butyl-[(5-fluorothiazol-2-yl)methoxy]-dimethyl-silane (680 mg, 2.75 mmol, 3% yield) as yellow oil. ¹H NMR (CDCl₃, 400 MHz) δ=7.25-7.20 (m, 1H), 4.81 (d, J=2.4 Hz, 2H), 0.95 (s, 9H), 0.18-0.11 (m, 6H).

### Step 3: Preparation of (5-fluorothiazol-2-yl)methanol

To a solution of tert-butyl-[(5-fluorothiazol-2-yl)methoxy]-dimethyl-silane (680 mg, 2.75 mmol, 1 eq) in THF (6 mL) was added TBAF (1 M, 6 mL, 2.18 eq). The mixture was stirred at 25° C. for 0.5 hour. TLC (PE:EA=3:1) indicated tert-butyl-[(5-fluorothiazol-2-yl)methoxy]-dimethyl-silane was consumed completely and one new spot formed. The reaction mixture was quenched by addition water (40 mL) at 10° C. and then diluted with EA 10 mL and extracted with EA (20 mL*2). The combined organic layers were washed with water (20 mL*2), dried over Na₂SO₄, filtered, and concentrated under reduced pressure to give a residue. The residue was purified by prep-TLC (SiO₂, PE:EA=3:1) to give (5-fluorothiazol-2-yl)methanol (230 mg, 1.73 mmol, 63% yield) as a yellow oil. ¹H NMR (DMSO-d₆, 400 MHz) δ=7.49 (d, J=3.2 Hz, 1H), 6.20-6.10 (m, 1H), 4.59 (dd, J=2.8, 6.0 Hz, 2H).

### Step 4: Preparation of 2-[(2-chloro-4-nitro-phenoxy)methyl]-5-fluoro-thiazole

To a solution of (5-fluorothiazol-2-yl)methanol (220 mg, 1.65 mmol, 1 eq) in ACN (4 mL) was added KOH (139 mg, 2.48 mmol, 1.5 eq) and 2-chloro-1-fluoro-4-nitro-benzene (290 mg, 1.65 mmol, 1 eq). The mixture was stirred at 25° C. for 0.5 hour. LC-MS showed (5-fluorothiazol-2-yl)methanol was consumed completely and 93.1% of desired mass was detected. The reaction mixture was quenched by addition sat. NH₄Cl (100 mL) at 10° C., and solid was formed and the solid was filtered and concentrated under reduced pressure to give 2-[(2-chloro-4-nitro-phenoxy)methyl]-5-fluoro-thiazole (420 mg, 1.45 mmol, 88% yield) as a yellow oil. 1H NMR (DMSO-d₆, 400 MHz) δ=8.37 (d, J=2.8 Hz, 1H), 8.27 (dd, J=2.8, 9.2 Hz, 1H), 7.70 (d, J=3.2 Hz, 1H), 7.53 (d, J=9.2 Hz, 1H), 5.60 (d, J=2.4 Hz, 2H).

### Step 5: Preparation of 3-chloro-4-[(5-fluorothiazol-2-yl)methoxy]aniline

To a solution of 2-[(2-chloro-4-nitro-phenoxy)methyl]-5-fluoro-thiazole (420 mg, 1.45 mmol, 1 eq) in THF (10 mL) and H₂O (1 mL) was added NH₄Cl (1.56 g, 29.1 mmol, 20 eq) and copper; zinc (3.75 g, 29.1 mmol, 20 eq) at 0° C. The mixture was stirred at 25° C. for 0.5 hour. LC-MS showed 2-[(2-chloro-4-nitro-phenoxy)methyl]-5-fluoro-thiazole was consumed completely and 89% of desired mass was detected. TLC (PE:EA=3:1) indicated 2-[(2-chloro-4-nitro-phenoxy)methyl]-5-fluoro-thiazole was consumed completely and one new spot formed. The reaction mixture was filtered and concentrated under reduced pressure to give a residue. The residue was dissolved into THF (20 mL). The reaction mixture was quenched by addition water (50 mL) at 10° C., and then diluted with EA (20 mL) and extracted with EA (20 mL*2). The combined organic layers were washed with water (20 mL*2), dried over Na₂SO₄, filtered and concentrated under reduced pressure to give 3-chloro-4-[(5-fluorothiazol-2-yl)methoxy]aniline (370 mg, 1.43 mmol, 98% yield) as a yellow solid. ¹H NMR (DMSO-d₆, 400 MHz) δ=7.62 (d, J=3.2 Hz, 1H), 6.97 (d, J=8.8 Hz, 1H), 6.64 (d, J=2.8 Hz, 1H), 6.47 (dd, J=2.8, 8.8 Hz, 1H), 5.17 (d, J=2.4 Hz, 2H), 5.03 (s, 2H).

### Step 6: Preparation of 1-[4-[4-[3-chloro-4-[(5-fluorothiazol-2-yl)methoxy]anilino]-7H-pyrrolo[2,3-d]pyrimidin-5-yl]-1-piperidyl]prop-2-en-1-one

To a solution of 3-chloro-4-[(5-fluorothiazol-2-yl)methoxy]aniline (370 mg, 1.43 mmol, 1 eq) in DMF (4 mL) and MeCN (4 mL) was added TsOH (493 mg, 2.86 mmol, 2 eq) and 1-[4-(4-chloro-7H-pyrrolo[2,3-d]pyrimidin-5-yl)-1-piperidyl]prop-2-en-1-one (416 mg, 1.43 mmol, 1 eq). The mixture was stirred at 70° C. for 12 hours. LC-MS showed 3-chloro-4-[(5-fluorothiazol-2-yl)methoxy]aniline was consumed completely and 59% of desired mass was detected. The reaction mixture was quenched by addition NaHCO₃ (150 mL) at 5° C. and solid was formed and the solid was filtered and concentrated under reduced pressure to give a residue. The residue was purified by prep-HPLC (neutral condition; column: Waters Xbridge C18 150*50 mm*10 um; mobile phase: [water(NH₄HCOs)-ACN]; B %: 32%-62%, 10 min) to afford 1-[4-[4-[3-chloro-4-[(5-fluorothiazol-2-yl)methoxy]anilino]-7H-pyrrolo[2,3-d]pyrimidin-5-yl]-1-piperidyl]prop-2-en-1-one (190 mg, 359 μmol, 25% yield, 96.9% purity) as an off-white solid. ¹H NMR (METHANOL-d₄, 400 MHz) δ=8.16 (s, 1H), 7.75 (d, J=2.4 Hz, 1H), 7.50-7.40 (m, 2H), 7.20 (d, J=8.8 Hz, 1H), 7.02 (s, 1H), 6.80 (dd, J=10.8, 16.8 Hz, 1H), 6.20 (dd, J=2.0, 16.8 Hz, 1H), 5.74 (dd, J=2.0, 10.8 Hz, 1H), 5.34 (d, J=2.4 Hz, 2H), 4.69 (br d, J=13.6 Hz, 1H), 4.27-4.17 (m, 1H), 3.37 (br d, J=14.4 Hz, 2H), 2.99-2.87 (m, 1H), 2.20-2.15 (m, 2H), 2.03 (s, 1H), 1.67-1.50 (m, 2H).

### Example 28: Preparation of 1-(4-(4-((3-chloro-4-(thieno[3,2-c]pyridin-6-ylmethoxy)phenyl)amino)-7H-pyrrolo[2,3-d]pyrimidin-5-yl)piperidin-1-yl)prop-2-en-1-one (Compound 224)

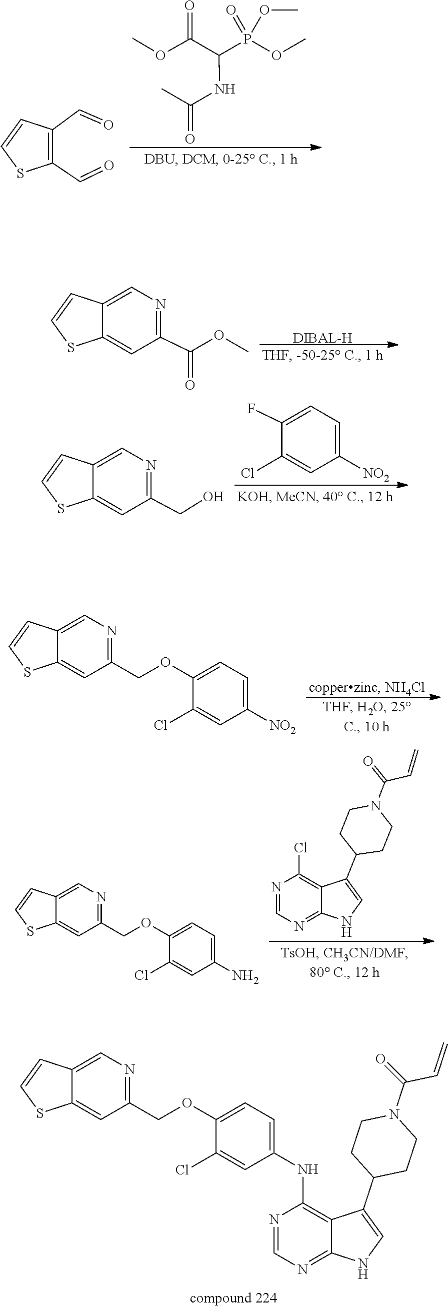

### Step 1: Preparation of Methyl thieno[3,2-c]pyridine-6-carboxylate

To a solution of thiophene-2,3-dicarbaldehyde (1 g, 7.13 mmol, 1 eq) in DCM (20 mL) at 0° C., then the mixture was added DBU (1.19 g, 7.85 mmol, 1.18 mL, 1.1 eq) and methyl 2-acetamido-2-dimethoxyphosphoryl-acetate (1.71 g, 7.13 mmol, 1 eq). The mixture was stirred at 25° C. for 1 hour. LC-MS showed no thiophene-2,3-dicarbaldehyde remained. Several new peaks were shown on LC-MS and desired compound was detected. The reaction mixture was diluted with water (50 mL) and extracted with EA (30 mL*3). The combined organic layers were washed with brine (30 mL*2), dried over Na₂SO₄, filtered, and concentrated under reduced pressure to give a residue. The residue was purified by column chromatography (SiO₂, Petroleum ether/Ethyl acetate-15/1 to 1/1, Rf=0.3) to give methyl thieno[3,2-c]pyridine-6-carboxylate (300 mg, 1.55 mmol, 21.76% yield) as a yellow solid. 1H NMR (400 MHz, DMSO-d₆) δ=9.25 (d, J=1.2 Hz, 1H), 8.86 (s, 1H), 8.16 (d, J=5.4 Hz, 1H), 7.87-7.61 (m, 1H), 3.92 (s, 3H).

### Step 2: Preparation of thieno[3,2-c]pyridin-6-ylmethanol

To a solution of methyl thieno[3,2-c]pyridine-6-carboxylate (290 mg, 1.50 mmol, 1 eq) in THF (5 mL) was added DIBAL-H (1 M, 3.00 mL, 2 eq) at −50° C. The mixture was stirred at 25° C. for 1 hour under N₂ atmosphere. LC-MS showed no thieno[3,2-c]pyridine-6-carboxylate remained. Several new peaks were shown on LC-MS and 30% of desired compound was detected. The reaction mixture was diluted with ice water (30 mL) and extracted with EA (15 mL*3). The combined organic layers were washed with brine (15 mL*2), dried over Na₂SO₄, filtered and concentrated under reduced pressure to give a residue. The residue was purified by prep-TLC (SiO₂, PE:EA=1:1, Rf=0.2) to give thieno[3,2-c]pyridin-6-ylmethanol (70 mg, 424 μmol, 28.2% yield) as a white solid. 1H NMR (400 MHz, DMSO-d₆) δ=9.06 (s, 1H), 8.07 (s, 1H), 7.82 (d, J=5.6 Hz, 1H), 7.59 (d, J=5.6 Hz, 1H), 5.48 (t, J=5.8 Hz, 1H), 4.69 (d, J=5.8 Hz, 2H).

### Step 3: Preparation of 6-((2-chloro-4-nitrophenoxy)methyl)thieno[3,2-c]pyridine

To a solution of thieno[3,2-c]pyridin-6-ylmethanol (65 mg, 393 μmol, 1 eq) and 2-chloro-1-fluoro-4-nitro-benzene (69.1 mg, 393 μmol, 1 eq) in ACN (5 mL) was added KOH (27.6 mg, 492 μmol, 1.25 eq). The mixture was stirred at 40° C. for 12 hours. LC-MS showed no thieno[3,2-c]pyridin-6-ylmethanol remained. Several new peaks were shown on LC-MS and ˜65% of desired compound was detected. The mixture was added into water (20 mL), then suspension was filtered, The filter cake was vacuum-dried to obtain the coarse product to give 6-((2-chloro-4-nitrophenoxy)methyl)thieno[3,2-c]pyridine (90 mg, 281 μmol, 71.3% yield) as a white solid.

### Step 4: Preparation of 3-chloro-4-(thieno[3,2-c]pyridin-6-ylmethoxy)aniline

To a solution of 6-[(2-chloro-4-nitro-phenoxy)methyl]thieno[3,2-c]pyridine (85 mg, 265 μmol, 1 eq) in THF (3 mL) and H₂O (3 mL) was added copper; zinc (342 mg, 2.65 mmol, 10 eq) and NH₄Cl (142 mg, 2.65 mmol, 10 eq). The mixture was stirred at 25° C. for 1 hour. LC-MS showed no 6-[(2-chloro-4-nitro-phenoxy)methyl]thieno[3,2-c]pyridine remained. Several new peaks were shown on LC-MS and ˜60% of desired compound was detected. The mixture was filtrated, and the filtrate was extracted with ethyl acetate (50 mL*3). The combined organic phase was washed with brine (10 mL*3), dried with anhydrous Na₂SO₄, filtered and concentrated in vacuum to give 3-chloro-4-(thieno[3,2-c]pyridin-6-ylmethoxy)aniline (60 mg, 206 μmol, 77.9% yield) as a white solid.

### Step 5: Preparation of 1-(4-(4-((3-chloro-4-(thieno[3,2-c]pyridin-6-ylmethoxy)phenyl)amino)-7H-pyrrolo[2,3-d]pyrimidin-5-yl)piperidin-1-yl)prop-2-en-1-one

To a solution of 3-chloro-4-(thieno[3,2-c]pyridin-6-ylmethoxy)aniline (50 mg, 172 μmol, 1 eq) in DMF (2 mL) and ACN (2 mL) was added TsOH (88.8 mg, 516 μmol, 3 eq) and 1-[4-(4-chloro-7H-pyrrolo[2,3-d]pyrimidin-5-yl)-1-piperidyl]prop-2-en-1-one (50.0 mg, 172 μmol, 1 eq). The mixture was stirred at 80° C. for 12 hours. LC-MS showed no 3-chloro-4-(thieno[3,2-c]pyridin-6-ylmethoxy)aniline remained. Several new peaks were shown on LC-MS and ˜40% of desired compound was detected. The mixture was added NaHCO₃ to adjust pH=7. Then the reaction mixture was diluted with water 50 mL and extracted with EA (15 mL*3). The combined organic layers were washed with brine (15 mL*2), dried over Na₂SO₄, filtered, and concentrated under reduced pressure to give a residue. The residue was purified by prep-HPLC (neutral condition, column: Waters Xbridge C18 150*50 mm*10 um; mobile phase: [water(NH₄HCOs)-ACN]; B %: 37%-67%, 9 min) to give 1-(4-(4-((3-chloro-4-(thieno[3,2-c]pyridin-6-ylmethoxy)phenyl)amino)-7H-pyrrolo[2,3-d]pyrimidin-5-yl)piperidin-1-yl)prop-2-en-1-one (10 mg, 17.4 μmol, 10.1% yield, 95% purity) was as a white solid. 1H NMR (400 MHz, DMSO-d₆) δ=11.59 (br s, 1H), 9.17 (s, 1H), 8.20 (d, J=18.6 Hz, 2H), 8.01-7.80 (m, 3H), 7.64 (d, J=5.4 Hz, 1H), 7.59-7.48 (m, 1H), 7.26 (d, J=9.0 Hz, 1H), 7.04 (s, 1H), 6.92-6.72 (m, 1H), 6.20-5.98 (m, 1H), 5.73-5.57 (m, 1H), 5.39 (s, 2H), 4.62-4.48 (m, 1H), 4.24-4.04 (m, 1H), 3.61-3.41 (m, 1H), 3.28-3.21 (m, 1H), 2.93-2.77 (m, 1H), 2.08-1.98 (m, 2H), 1.51-1.35 (m, 2H).

### Example 29: Preparation of 1-[4-[4-[3-chloro-4-[3-(trifluoromethyl)phenoxy]anilino]-7H-pyrrolo[2,3-d]pyrimidin-5-yl]-1-piperidyl]prop-2-en-1-one (Compound 225)

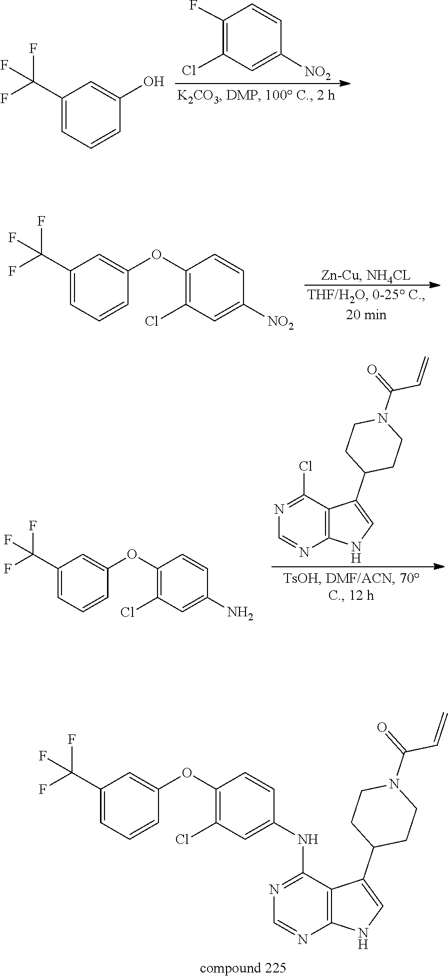 [SMILES: Oc1cccc(C(F)(F)F)c1 ; O=[N+]([O-])c1ccc(F)c(Cl)c1 ; O=[N+]([O-])c1ccc(Oc2cccc(C(F)(F)F)c2)c(Cl)c1 ; Nc1ccc(Oc2cccc(C(F)(F)F)c2)c(Cl)c1 ; C=CC(=O)N1CCC(c2c[nH]c3ncnc(Cl)c23)CC1 ; C=CC(=O)N1CCC(c2c[nH]c3ncnc(Nc4ccc(Oc5cccc(C(F)(F)F)c5)c(Cl)c4)c23)CC1]

### Step 1: Preparation of 2-chloro-4-nitro-1-[3-(trifluoromethyl)phenoxy]benzene

To a solution of 3-(trifluoromethyl)phenol (1 g, 6.17 mmol, 741 μL, 1 eq) in DMF (10 mL) was added K₂CO₃ (2.56 g, 18.5 mmol, 3 eq) and 2-chloro-1-fluoro-4-nitro-benzene (1.08 g, 6.17 mmol, 1 eq). The mixture was stirred at 100° C. for 2 hours. LC-MS showed 3-(trifluoromethyl)phenol (1 g, 6.17 mmol, 741 μL, 1 eq) was consumed completely and the compound had no signal on LCMS. The residue was poured into ice-water (20 mL) and stirred for 5 minutes. The aqueous phase was extracted with ethyl acetate (20 mL*3). The combined organic phase was washed with brine (10 mL*2), dried with anhydrous Na₂SO₄, filtered and concentrated in vacuum to give 2-chloro-4-nitro-1-[3-(trifluoromethyl)phenoxy]benzene (1.67 g, 5.25 mmol, 85.1% yield) as a yellow solid. ¹H NMR (400 MHz, DMSO-d₆) δ=8.54 (d, J=2.8 Hz, 1H), 8.25 (dd, J=2.8, 9.2 Hz, 1H), 7.76 (d, J=8.0 Hz, 1H), 7.73-7.69 (m, 1H), 7.64 (s, 1H), 7.55-7.51 (m, 1H), 7.24 (d, J=9.2 Hz, 1H).

### Step 2: Preparation of 3-chloro-4-[3-(trifluoromethyl)phenoxy]aniline

To a solution of 2-chloro-4-nitro-1-[3-(trifluoromethyl)phenoxy]benzene (1 g, 3.15 mmol, 1 eq) in H₂O (3 mL) and THF (20 mL) was added copper; zinc (8.12 g, 63.0 mmol, 20 eq) and NH₄Cl (3.37 g, 63.0 mmol, 20 eq) at 0° C. The mixture was stirred at 25° C. for 20 minutes. LC-MS showed 2-chloro-4-nitro-1-[3-(trifluoromethyl)phenoxy]benzene was consumed completely and 98.4% of desired mass was detected. The residue was poured into ice-water (20 mL) and stirred for 5 minutes. The aqueous phase was extracted with ethyl acetate (20 mL*3). The combined organic phase was washed with brine (10 mL*2), dried with anhydrous Na₂SO₄, filtered and concentrated in vacuum to give 3-chloro-4-[3-(trifluoromethyl)phenoxy]aniline (890 mg, 3.09 mmol, 98.3% yield) as a yellow solid. ¹H NMR (DMSO, 400 MHz) δ=7.58-7.51 (m, 1H), 7.37 (d, J=7.6 Hz, 1H), 7.09 (br d, J=8.4 Hz, 1H), 7.06 (s, 1H), 6.99 (d, J=8.8 Hz, 1H), 6.75 (d, J=2.4 Hz, 1H), 6.59 (dd, J=2.8, 8.8 Hz, 1H), 5.43 (s, 2H).

### Step 3: Preparation of 1-[4-[4-[3-chloro-4-[3-(trifluoromethyl)phenoxy]anilino]-7H-pyrrolo[2,3-d]pyrimidin-5-yl]-1-piperidyl]prop-2-en-1-one

To a solution of 3-chloro-4-[3-(trifluoromethyl)phenoxy]aniline (100 mg, 348 μmol, 1 eq) in DMF (1 mL) and ACN (1 mL) was added TsOH (120 mg, 695 μmol, 2 eq) and 1-[4-(4-chloro-7H-pyrrolo[2,3-d]pyrimidin-5-yl)-1-piperidyl]prop-2-en-1-one (91.0 mg, 313 μmol, 0.9 eq). The mixture was stirred at 70° C. for 12 hours. LC-MS showed 3-chloro-4-[3-(trifluoromethyl)phenoxy]aniline was consumed completely. Several new peaks were shown on LC-MS showed 48.4% compound was detected. The residue was poured into ice-water (20 mL) and stirred for 5 minutes. The aqueous phase was extracted with ethyl acetate (20 mL*3). The combined organic phase was washed with brine (10 mL*2), dried with anhydrous Na₂SO₄, filtered, and concentrated in vacuum. The residue was purified by prep-HPLC (column: Waters Xbridge 150*25 mm*5 um; mobile phase: [water(NH₄HCO₃)-ACN]; B %: 52%-82%, 9 min) to give 1-[4-[4-[3-chloro-4-[3-(trifluoromethyl)phenoxy]anilino]-7H-pyrrolo[2,3-d]pyrimidin-5-yl]-1-piperidyl]prop-2-en-1-one (131 mg, 236 μmol, 68.0% yield, 97.7% purity) as a white solid. ¹H NMR (400 MHz, MeOD) δ=8.26-8.21 (m, 1H), 7.95-7.90 (m, 1H), 7.61-7.52 (m, 2H), 7.39 (d, J=7.6 Hz, 1H), 7.25-7.18 (m, 3H), 7.11 (s, 1H), 6.82 (dd, J=10.4, 16.8 Hz, 1H), 6.21 (dd, J=2.0, 16.8 Hz, 1H), 5.75 (dd, J=2.0, 10.4 Hz, 1H), 4.75-4.66 (m, 1H), 4.28-4.20 (m, 1H), 3.52-3.44 (m, 1H), 3.43-3.36 (m, 1H), 3.02-2.91 (m, 1H), 2.27-2.16 (m, 2H), 1.70-1.55 (m, 2H).

### Example 30: Preparation of 1-(4-(4-((1-(3-fluorobenzyl)-1H-indazol-5-yl)amino)-7H-pyrrolo[2,3-d]pyrimidin-5-yl)piperidin-1-yl)prop-2-en-1-one (Compound 226)

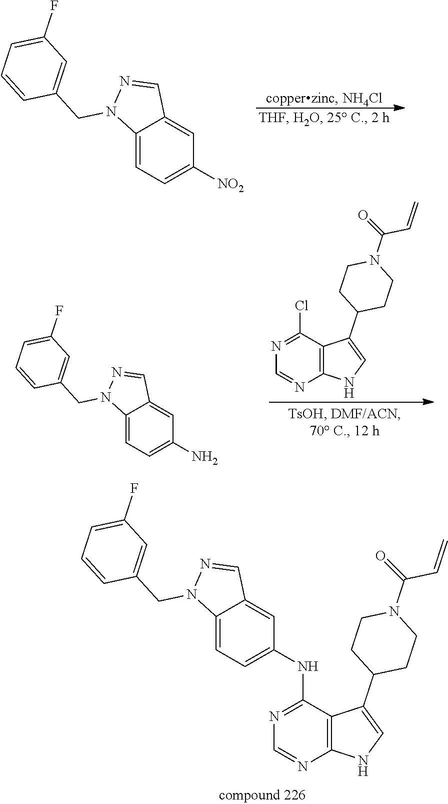

### Step 1: Preparation of 1-(3-fluorobenzyl)-1H-indazol-5-amine

To a solution of 1-[(3-fluorophenyl)methyl]-5-nitro-indazole (600 mg, 2.21 mmol, 1 eq) in THF (10 mL) and H₂O (10 mL) was added copper; zinc (2.85 g, 22.1 mmol, 10 eq) and NH₄Cl (1.18 g, 22.1 mmol, 10 eq). The mixture was stirred at 25° C. for 2 hours. LC-MS showed no 1-[(3-fluorophenyl)methyl]-5-nitro-indazole. Several new peaks were shown on LC-MS and 97% of desired compound was detected. The mixture was filtrated, and the filtrate was extracted with ethyl acetate (50 mL*3). The combined organic phase was washed with brine (10 mL*3), dried with anhydrous Na₂SO₄, filtered, and concentrated in vacuum to give 1-(3-fluorobenzyl)-1H-indazol-5-amine (450 mg, 1.87 mmol, 84.3% yield) as a yellow solid. 1H NMR (400 MHz, DMSO-d₆) δ=7.78 (s, 1H), 7.38 (d, J=8.8 Hz, 2H), 7.12-7.03 (m, 1H), 6.99 (br d, J=7.8 Hz, 2H), 6.87-6.73 (m, 2H), 5.55 (s, 2H), 4.84 (s, 2H).

### Step 2: Preparation of 1-(4-(4-((1-(3-fluorobenzyl)-1H-Indazol-5-yl)amino)-7H-pyrrolo[2,3-d]pyrimidin-8-yl)piperidin-1-yl)prop-2-en-1-one

To a solution of 1-[(3-fluorophenyl)methyl]indazol-5-amine (400 mg, 1.66 mmol, 1 eq) in DMF (6 mL) and ACN (6 mL) was added 1-[4-(4-chloro-7H-pyrrolo[2,3-d]pyrimidin-5-yl)-1-piperidyl]prop-2-en-1-one (482 mg, 1.66 mmol, 1 eq) and TsOH (857 mg, 4.97 mmol, 3 eq). The mixture was stirred at 70° C. for 12 hours. LC-MS showed 1-[4-(4-chloro-7H-pyrrolo[2,3-d]pyrimidin-5-yl)-1-piperidyl]prop-2-en-1-one remained. Several new peaks were shown on LC-MS and 60% of desired compound was detected. The mixture was added NaHCO₃ to adjust pH=7. Then the reaction mixture was diluted with water (30 mL) and extracted with EA (30 mL*3). The combined organic layers were washed with brine (30 mL*2), dried over Na₂SO₄, filtered, and concentrated under reduced pressure to give a residue. The residue was purified by prep-HPLC (neutral condition; column: Waters Xbridge 150*25 mm*5 um; mobile phase: [water(NH₄HCO₃)-ACN]; B %: 33%-63%, 10 min) to give 1-(4-(4-((1-(3-fluorobenzyl)-1H-indazol-5-yl)amino)-7H-pyrrolo[2,3-d]pyrimidin-5-yl)piperidin-1-yl)prop-2-en-1-one (195 mg, 374 μmol, 22.6% yield, 95% purity) as a yellow solid. 1H NMR (400 MHz, DMSO-d₆) δ=11.56 (s, 1H), 8.13 (d, J=9.2 Hz, 2H), 8.09-8.03 (m, 2H), 7.69 (d, J=9.0 Hz, 1H), 7.58-7.52 (m, 1H), 7.37 (d, J=6.2 Hz, 1H), 7.13-7.07 (m, 1H), 7.06-7.00 (m, 3H), 6.94-6.72 (m, 1H), 6.19-6.02 (m, 1H), 5.75-5.59 (m, 3H), 4.56 (br d, J=11.0 Hz, 1H), 4.14 (br d, J=12.2 Hz, 1H), 3.55-3.45 (m, 1H), 3.31-3.21 (m, 1H), 2.88-2.77 (m, 1H), 2.10-2.01 (m, 2H), 1.52-1.37 (m, 2H).

### Example 31: Preparation of 1-(4-(4-((3-chloro-4-(pyridin-2-ylmethoxy-d2)phenyl)amino)-7H-pyrrolo[2,3-d]pyrimidin-5-yl)piperidin-1-yl)prop-2-en-1-one (Compound 227)

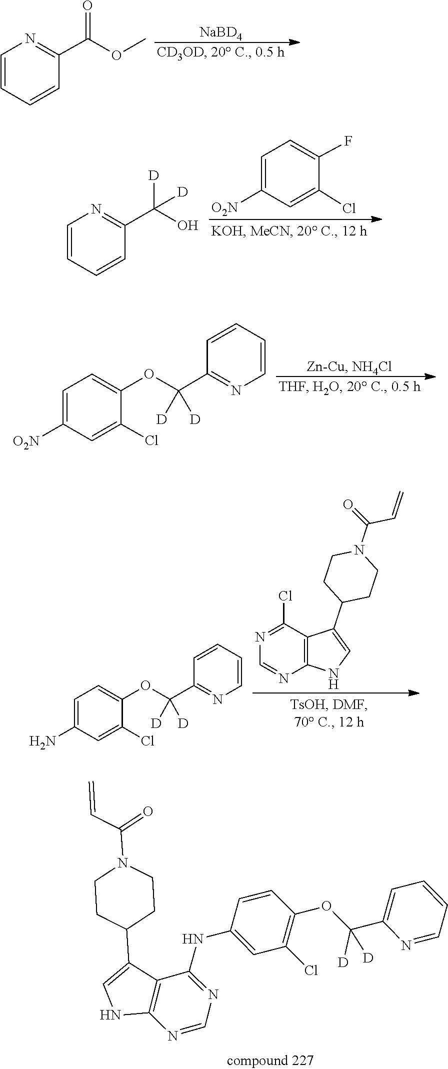

### Step 1: Preparation of pyridin-2-ylmethan-d2-ol

To a solution of methyl picolinate (2 g, 14.6 mmol, 1.75 mL, 1 eq) in CD₃OD (15 mL) was added sodium; tetradeuterioboranuide (1.10 g, 29.2 mmol, 2 eq). The mixture was stirred at 20° C. for 0.5 hour. TLC (PE:EA=0:1, R_{f}=0.15) indicated none of methyl picolinate was remained and one major new spot with larger polarity was detected. The reaction mixture was partitioned between water (100 mL) and EA (100 mL). The organic phase was separated, washed with water 300 mL (100 ml*3) and concentrated under reduced pressure to give pyridin-2-ylmethan-d2-ol (1.51 g, 13.6 mmol, 93.2% yield) as a yellow liquid. ¹H NMR (DMSO-d₆, 400 MHz) δ=8.50-8.43 (m, 1H), 7.87-7.71 (m, 1H), 7.48-7.44 (m, 1H), 7.28-7.16 (m, 1H), 5.35 (s, 1H).

### Step 2: Preparation of 2-((2-chloro-4-nitrophenoxy)methyl-d2)pyridine

To a solution of dideuterio(2-pyridyl)methanol (1.5 g, 13.5 mmol, 1 eq) in MeCN (15 mL) was added KOH (1.14 g, 20.3 mmol, 1.5 eq). The mixture was stirred at 20° C. for 0.5 hour. Then 2-chloro-1-fluoro-4-nitrobenzene (2.37 g, 13.5 mmol, 1 eq) was added to the above mixture. The resulting mixture was stirred at 20° C. for 12 hours. TLC (PE:EA=1:1, R_{f}=0.15) indicated none of dideuterio(2-pyridyl)methanol was remained and one major new spot with lower polarity was detected. The reaction mixture was partitioned between water (50 mL) and (EA 50 mL). The organic phase was separated, washed with water (50 mL*2) and concentrated under reduced pressure to give a residue. The crude product was triturated with EA (5 mL) at 20° C. for 5 minutes to give 2-((2-chloro-4-nitrophenoxy)methyl-d2)pyridine (2.63 g, 9.86 mmol, 73.1% yield, 100% purity) as a yellow solid. ¹H NMR (DMSO-d₆, 400 MHz) δ=8.62-8.59 (m, 1H), 8.35 (d, J=2.8 Hz, 1H), 8.26-8.20 (m, 1H), 7.92-7.85 (m, 1H), 7.57 (d, J=8.0 Hz, 1H), 7.47 (d, J=9.2 Hz, 1H), 7.42-7.35 (m, 1H).

### Step 3: Preparation of 3-chloro-4-(pyridin-2-ylmethoxy-d2)aniline

To a solution of 2-[(2-chloro-4-nitro-phenoxy)-dideuterio-methyl]pyridine (2.6 g, 9.75 mmol, 1 eq) in THF (15 mL) and H₂O (15 mL) was added copper; zinc (12.6 g, 97.5 mmol, 10 eq) and NH₄Cl (5.22 g, 97.5 mmol, 10 eq). The mixture was stirred at 20° C. for 0.5 hour. TLC (PE:EA=1:1, R_{f}=0.14) indicated no 2-[(2-chloro-4-nitro-phenoxy)-dideuterio-methyl]pyridine was remained and one major new spot with larger polarity was detected. The reaction mixture was filtered, and the filtrate was partitioned between water (60 mL) and EA (60 mL). The organic phase was separated and washed with water (50 mL*2), concentrated under reduced pressure to give 3-chloro-4-(pyridin-2-ylmethoxy-d2)aniline (1.7 g, 7.15 mmol, 73.3% yield, 99.5% purity) as a yellow solid. ¹H NMR (DMSO-d₆, 400 MHz) δ=8.55 (d, J=4.4 Hz, 1H), 7.88-7.77 (m, 1H), 7.53 (d, J=8.0 Hz, 1H), 7.37-7.29 (m, 1H), 6.90 (d, J=8.8 Hz, 1H), 6.66 (d, J=2.8 Hz, 1H), 6.51-6.41 (m, 1H), 5.08-5.04 (m, 1H), 4.96-4.90 (m, 2H).

### Step 4: Preparation of 1-(4-(4-((3-chloro-4-(pyridin-2-ylmethoxy-d2)phenyl)amino)-7H-pyrrolo[2,3-d]pyrimidin-5-yl)piperidin-1-yl)prop-2-en-1-one

To a solution of 3-chloro-4-[dideuterio(2-pyridyl)methoxy]aniline (407 mg, 1.72 mmol, 1 eq) and 1-(4-(4-chloro-7H-pyrrolo[2,3-d]pyrimidin-5-yl)piperidin-1-yl)prop-2-en-1-one (500 mg, 1.72 mmol, 1 eq) in DMF (10 mL) was added TsOH (592 mg, 3.44 mmol, 2 eq). The mixture was stirred at 70° C. for 12 hours. TLC (PE:EA=0:1, R_{f}=0.06) indicated none of 3-chloro-4-[dideuterio(2-pyridyl)methoxy]aniline was remained and one major new spot with larger polarity was detected. The reaction mixture was partitioned between NaHCO₃ (50 mL) and EA (50 mL). The organic phase was separated and washed with NaHCO₃ (50 mL*2), concentrated under reduced pressure to give a residue. The residue was purified by prep-TLC (SiO₂, PE:EA=0:1) to give 1-(4-(4-((3-chloro-4-(pyridin-2-ylmethoxy-d2)phenyl)amino)-7H-pyrrolo[2,3-d]pyrimidin-5-yl)piperidin-1-yl)prop-2-en-1-one (250 mg, 488 μmol, 28.4% yield, 95.9% purity) as a white solid. ¹H NMR (DMSO-d₆, 400 MHz) δ=11.61 (br s, 1H), 8.60 (br d, J=4.8 Hz, 1H), 8.19 (s, 1H), 7.99 (s, 1H), 7.92-7.81 (m, 2H), 7.63-7.50 (m, 2H), 7.41-7.34 (m, 1H), 7.22 (d, J=9.2 Hz, 1H), 7.05 (s, 1H), 6.90-6.76 (m, 1H), 6.16-6.03 (m, 1H), 5.72-5.60 (m, 1H), 4.57 (br d, J=11.6 Hz, 1H), 4.15 (br d, J=12.0 Hz, 1H), 3.52 (br t, J=11.6 Hz, 1H), 3.30-3.23 (m, 1H), 2.90-2.80 (m, 1H), 2.11-1.96 (m, 2H), 1.44 (br d, J=12.0 Hz, 2H).

### Example 32: Preparation of 1-(4-(4-((3-chloro-4-((3-fluorobenzyl)oxy)phenyl)amino)-7H-pyrrolo[2,3-d]pyrimidin-5-yl)piperidin-1-yl)prop-2-en-1-one (Compound 228)

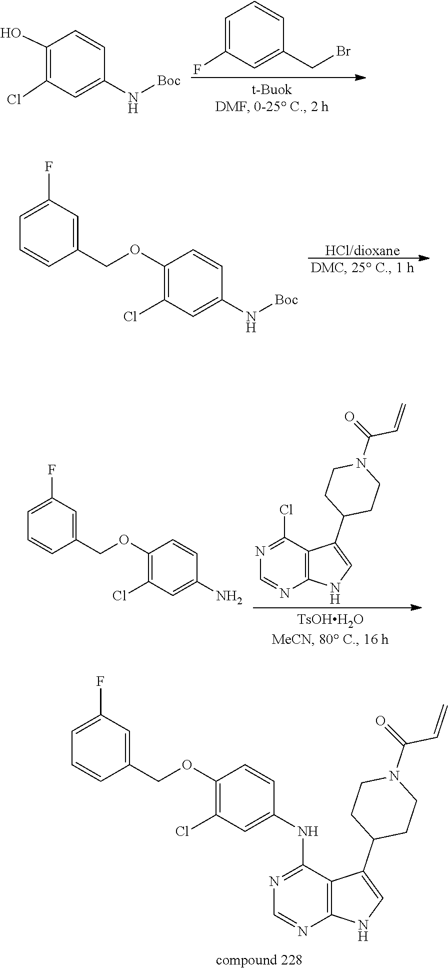

### Step 1: Preparation of tert-butyl (3-chloro-4-((3-fluorobenzyl)oxy)phenyl)carbamate

To a solution of tert-butyl (3-chloro-4-hydroxyphenyl)carbamate (2.86 g, 11.74 mmol) in DMF (30 mL) was added 1-(bromomethyl)-3-fluorobenzene (2.22 g, 11.74 mmol) and t-BuOK (2.63 g, 23.47 mmol) at 0° C. The mixture was stirred at 25° C. for 2 h. LCMS showed 37% peak with desired mass. The reaction mixture was diluted with H₂O (120 mL), extracted with ethyl acetate (150 mL×3). The combined organic layers were washed with brine (50 mL×2), dried over Na₂SO₄, filtered, and concentrated under reduced pressure to give a residue. The residue was purified by column chromatography (SiO₂, Petroleum ether/Ethyl acetate=3/1) to afford tert-butyl (3-chloro-4-((3-fluorobenzyl)oxy)phenyl)carbamate (2.2 g, 48.44% yield) as a white solid.

### Step 2: Preparation of 3-chloro-4-((3-fluorobenzyl)oxy)aniline

To a solution of tert-butyl (3-chloro-4-((3-fluorobenzyl)oxy)phenyl)carbamate (2.2 g, 6.25 mmol) in DCM (22 mL) was added HCl/dioxane (4 M, 22 mL). The mixture was stirred at 25° C. for 1 h. LCMS showed 57% peak with desired mass. The reaction mixture was concentrated under reduced pressure to give a residue. The residue was dissolved with water (100 mL), the mixture was adjusted to pH=8 by saturated NaHCO₃ solution. The mixture was extracted with dichloromethane (120 mL×3). The combined organic layers were washed with brine (30 mL×2), dried over Na₂SO₄, filtered and concentrated under reduced pressure to give crude product. The crude product was purified by column chromatography (SiO₂, Petroleum ether/Ethyl acetate=3/1) to afford 3-chloro-4-((3-fluorobenzyl)oxy)aniline (900 mg, 57.18% yield) as a brown solid.

### Step 3: Preparation of 1-(4-(4-((3-chloro-4-((3-fluorobenzyl)oxy)phenyl)amino)-7H-pyrrolo[2,3-d]pyrimidin-8-yl)piperidin-1-yl)prop-2-en-1-one

To a solution of 3-chloro-4-((3-fluorobenzyl)oxy)aniline (300 mg, 1.19 mmol) in MeCN (3 mL) was added 1-(4-(4-chloro-7H-pyrrolo[2,3-d]pyrimidin-5-yl)piperidin-1-yl)prop-2-en-1-one (346.56 mg, 1.19 mmol) and TsOH·H₂O (22.67 mg, 119.20 μmol). The mixture was stirred at 80° C. for 16 h. LCMS showed 65% peak with desired mass. After cooling to room temperature, the reaction mixture was concentrated under reduced pressure to give a residue. The residue was purified by prep-HPLC (basic conditions) and prep-TLC (SiO₂, ethyl acetate) to afford 1-(4-(4-((3-chloro-4-((3-fluorobenzyl)oxy)phenyl)amino)-7H-pyrrolo[2,3-d]pyrimidin-5-yl)piperidin-1-yl)prop-2-en-1-one (95.7 mg, 15.73% yield) as a white solid. ¹H NMR (400 MHz, DMSO-d₆) δ 11.60 (s, 1H), 8.18 (s, 1H), 7.99 (s, 1H), 7.83 (d, J=2.4 Hz, 1H), 7.56-7.52 (m, 2H), 7.48-7.46 (m, 2H), 7.34-7.32 (m, 2H), 7.23-7.20 (m, 2H), 7.05-7.04 (m, 1H), 6.85-6.80 (m, 1H), 6.10 (dd, J=2.8, 16.8 Hz, 1H), 5.67 (dd, J=2.4, 10.4 Hz, 1H), 5.25 (s, 2H), 4.55 (d, J=12.4, 1H), 4.14 (d, J=12.0, 1H), 3.52 (t, J=8.8, 1H), 3.29-3.24 (m, 1H), 2.83 (t, J=12.4 Hz, 1H), 2.05-2.01 (m, 2H), 1.48-1.42 (m, 2H).

### Example 33: Preparation of 1-(4-(4-((3-chloro-4-(isothiazol-3-ylmethoxy)phenyl)amino)-7H-pyrrolo[2,3-d]pyrimidin-5-yl)piperidin-1-yl)prop-2-en-1-one (Compound 229)

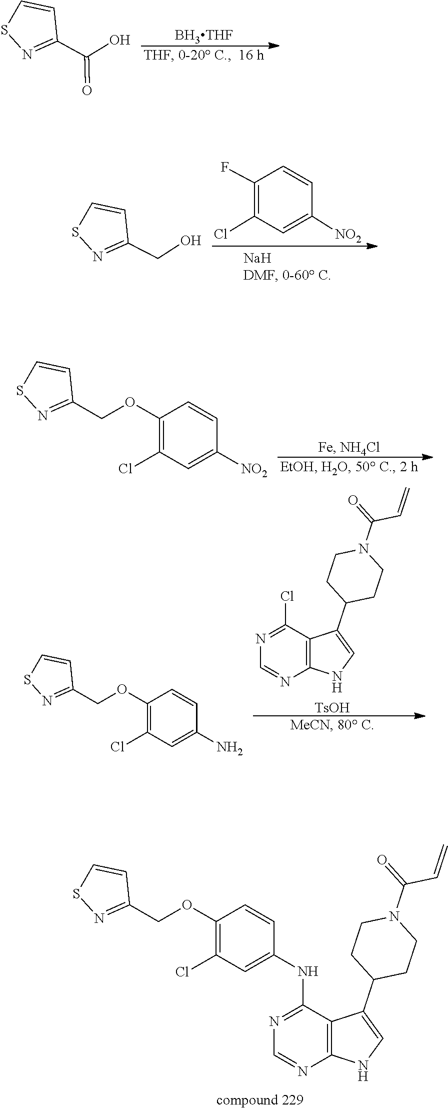

### Step 1: Preparation of isothiazol-3-ylmethanol

To a solution of isothiazole-3-carboxylic acid (250 mg, 1.94 mmol) in THF (3 mL) was added BH₃·THF (1 M, 2.90 mL) dropwise at 0° C. under N₂. The mixture was stirred at 25° C. for 16 h. TLC (Dichloromethane/Methanol=10/1) showed the starting material was consumed incompletely and a new spot was formed. The reaction was quenched with methanol (5 mL) and concentrated under reduced pressure to give residue. The residue was purified by column chromatography (SiO₂, Dichloromethane/Methanol=10/1) to afford isothiazol-3-ylmethanol (140 mg, 62.80% yield) as yellow oil. ¹H NMR (400 MHz, DMSO-d₆) δ 9.00 (d, J=4.8 Hz, 1H), 7.35 (d, J=4.8 Hz, 1H), 5.46 (t, J=6.0 Hz, 1H), 4.58 (d, J=6.0 Hz, 2H).

### Step 2: Preparation of 3-((2-chloro-4-nitrophenoxy)methyl)isothiazole

To a solution of isothiazol-3-ylmethanol (59.89 mg, 520.10 μmol) in THF (1 mL) was added NaH (37.82 mg, 945.63 μmol, 60% purity) at 0° C. The mixture was stirred at 0° C. for 0.5 h. The mixture was added 2-chloro-1-fluoro-4-nitro-benzene (83 mg, 472.81 μmol). The mixture was stirred at 60° C. for 16 h. TLC (Petroleum ether/Ethyl acetate=3/1) showed a new spot was formed. After cooling to room temperature, the reaction was quenched with NH₄Cl solution (10 mL) and extracted with ethyl acetate (10 mL×3), the combined organic layers were washed with brine (5 mL×2) and dried over Na₂SO₄, filtered, and concentrated to give a residue. The residue was purified by column chromatography (SiO₂, Petroleum ether/Ethyl acetate=3/1) to afford 3-((2-chloro-4-nitrophenoxy)methyl)isothiazole (88 mg, 68.76% yield) as a yellow solid.

### Step 3: Preparation of 3-chloro-4-(Isothiazol-3-ylmethoxy)aniline

To a mixture of 3-((2-chloro-4-nitrophenoxy)methyl)isothiazole (88 mg, 325.09 μmol) in EtOH (1 mL) and H₂O (0.1 mL) was added Fe (90.77 mg, 1.63 mmol) and NH₄Cl (86.95 mg, 1.63 mmol). The mixture was stirred at 50° C. for 2 h. LCMS showed 98% peak with desired mass. After cooling to room temperature, the reaction mixture was filtered and the filtrate was concentrated under to afford 3-chloro-4-(isothiazol-3-ylmethoxy)aniline (85 mg, crude) as a yellow solid.

### Step 4: Preparation of 1-(4-(4-((3-chloro-4-(Isothiazol-3-ylmethoxy)phenyl)amino)-7H-pyrrolo[2,3-d]pyrimidin-5-yl)piperidin-1-yl)prop-2-en-1-one

To a mixture of 3-chloro-4-(isothiazol-3-ylmethoxy)aniline (40 mg, 166.18 μmol) and 1-(4-(4-chloro-7H-pyrrolo[2,3-d]pyrimidin-5-yl)piperidin-1-yl)prop-2-en-1-one (48.32 mg, 166.18 μmol) in MeCN (1 mL) was added TsOH·H₂O (3.16 mg, 16.62 μmol). The mixture was stirred at 80° C. for 16 h. LCMS showed 44% peak with desired mass. After cooling to room temperature, the reaction was concentrated to give a residue. The mixture was purified by prep-HPLC (neutral conditions) to afford 1-(4-(4-((3-chloro-4-(isothiazol-3-ylmethoxy)phenyl)amino)-7H-pyrrolo[2,3-d]pyrimidin-5-yl)piperidin-1-yl)prop-2-en-1-one (6.5 mg, 15.93% yield) as a yellow solid. ¹H NMR (400 MHz, DMSO-d₆) δ 11.60 (s, 1H), 9.12 (d, J=4.4 Hz, 1H), 8.18 (s, 1H), 7.98 (s, 1H), 7.83 (d, J=2.4 Hz, 1H), 7.53 (dd, J=2.4, 9.2 Hz, 1H), 7.49 (d, J=4.8 Hz, 1H), 7.23 (d, J=8.8 Hz, 1H), 7.04 (d, J=1.6 Hz, 1H), 6.83 (dd, J=10.4, 16.4 Hz, 1H), 6.10 (dd, J=2.4, 16.4 Hz, 1H), 5.66 (dd, J=2.4, 10.4 Hz, 1H), 5.35 (s, 2H), 4.61-4.49 (m, 1H), 4.19-4.08 (m, 1H), 3.51 (d, J=2.8 Hz, 1H), 3.26-3.23 (m, 1H), 2.84 (s, 1H), 2.08-1.97 (m, 2H), 1.50-1.37 (m, 2H).

### Example 34: Preparation of 1-(4-(4-((3-chloro-4-(pyridin-2-ylmethoxy)phenyl)amino)-7H-pyrrolo[2,3-d]pyrimidin-5-yl)-2,2-dimethylpiperidin-1-yl)prop-2-en-1-one (Compound 230)

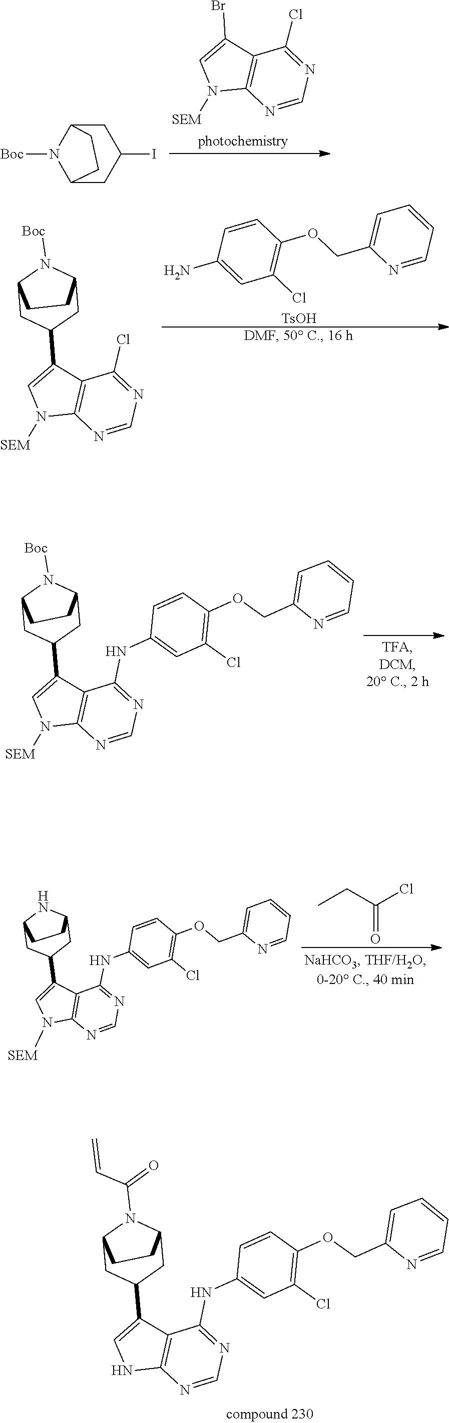

### Step 1: Preparation of tert-butyl 3-[4-chloro-7-(2-trimethylsilylethoxymethyl)pyrrolo[2,3-d]pyrimidin-5-yl]-8-azabicyclo[3.2.1]octane-8-carboxylate

The reaction was carried out vis photo-chemistry platform: To a 15 mL vial equipped with a stir bar was added Aryl halide (0.2 mmol. 1 eq), alkyl iodide (0.4 mmol, 2 eq), Ir[dF(CF₃)ppy]₂(dtbpy)(PF₆) (0.01 eq), NiCl₂·dtbbpy (0.015 eq), TTMSS (1.00 eq), Na₂CO₃ (2 eq) in DME (0.1 M). The vial was sealed and placed under nitrogen was added. The reaction was stirred and irradiated with a 10 W blue LED lamp (3 cm away), with cooling water to keep the reaction temperature at 25° C. for 14 h. The reaction mixture was concentrated under reduced pressure to give a residue. The residue was purified by column chromatography (SiO₂, Petroleum ether/Ethyl acetate=3/1) to afford tert-butyl 3-[4-chloro-7-(2-trimethylsilylethoxymethyl)pyrrolo[2,3-d]pyrimidin-5-yl]-8-azabicyclo[3.2.1]octane-8-carboxylate (290 mg, 29.00% yield) as a white solid.

### Step 2: Preparation of tert-butyl (1R,3r,5S)-3-(4-((3-chloro-4-(pyridin-2-ylmethoxy)phenyl)amino)-7-((2-(trimethylsilyl)ethoxy)methyl)-7H-pyrrolo[2,3-d]pyrimidin-5-yl)-8-azabicyclo[3.2.1]octane-8-carboxylate

To a mixture of tert-butyl 3-[4-chloro-7-(2-trimethylsilylethoxymethyl)pyrrolo[2,3-d]pyrimidin-5-yl]-8-azabicyclo[3.2.1]octane-8-carboxylate (120 mg, 243.35 μmol), 3-chloro-4-(2-pyridylmethoxy)aniline (57.11 mg, 243.35 μmol) in DMF (1 mL) was added TsOH (62.86 mg, 365.03 μmol). The mixture was stirred at 50° C. for 16 h. LCMS showed 40% peak with desired mass. After cooling to room temperature, the reaction mixture was poured into water (30 mL), extracted with ethyl acetate (20 mL×2). The combined organic layers were washed with brine (10 mL×2), dried over Na₂SO₄, filtered, and concentrated under reduced pressure to give a residue. The residue was purified by column chromatography (SiO₂, Ethyl acetate) to afford tert-butyl (1R,3r,5S)-3-(4-((3-chloro-4-(pyridin-2-ylmethoxy)phenyl)amino)-7-((2-(trimethylsilyl)ethoxy)methyl)-7H-pyrrolo[2,3-d]pyrimidin-5-yl)-8-azabicyclo[3.2.1]octane-8-carboxylate (90 mg, 53.50% yield) as a white solid.

### Step 3: Preparation of 5-((1R,3r,5S)-8-azabicyclo[3.2.1]octan-3-yl)-N-(3-chloro-4-(pyridin-2-ylmethoxy)phenyl)-7H-pyrrolo[2,3-d]pyrimidin-4-amine

To a solution of tert-butyl 3-[4-[3-chloro-4-(2-pyridylmethoxy)anilino]-7-(2-trimethylsilylethoxymethyl)pyrrolo[2,3-d]pyrimidin-5-yl]-8-azabicyclo[3.2.1]octane-8-carboxylate (50 mg, 72.32 μmol) in DCM (1.5 mL) was added TFA (0.5 mL). The mixture was stirred at 20° C. for 2 h. LCMS showed 82% with desired mass. The reaction mixture was concentrated under reduced pressure to afford 4-chloro-5-(2,2-dimethylpiperidin-4-yl)-7H-pyrrolo[2,3-d]pyrimidine (50 mg, crude, TFA salt) as yellow oil.

### Step 4: Preparation of 1-((1R,3r,5S)-3-(4-((3-chloro-4-(((Z)-2-(methyleneamino)but-2-en-1-yl)oxy)phenyl)amino)-7H-pyrrolo[2,3-d]pyrimidin-5-yl)-8-azabicyclo[3.2.1]octan-8-yl)prop-2-en-1-one

To a solution of 5-(8-azabicyclo[3.2.1]octan-3-yl)-N-[3-chloro-4-(2-pyridylmethoxy)phenyl]-7H-pyrrolo[2,3-d]pyrimidin-4-amine (47.52 mg, 82.64 μmol, TFA salt) in THF (0.5 mL) and H₂O (0.5 mL) was added NaHCO₃ (69.43 mg, 826.42 μmol) at 0° C. under N₂. The mixture was added prop-2-enoyl chloride (8.98 mg, 99.17 μmol) slowly. The mixture was stirred at 25° C. for 10 min under N₂. The mixture was added K₂CO₃ (22.84 mg, 165.28 μmol). The mixture was stirred at 25° C. for 30 min under N₂. LCMS showed 75% peak with desired mass. The reaction mixture was poured into water (20 mL), extracted with ethyl acetate (10 mL×2). The combined organic layers were washed with brine (10 mL×2), dried over Na₂SO₄, filtered, and concentrated under reduced pressure to give a residue. The residue was purified by prep-HPLC (neutral conditions) to afford 1-((1R,3r,5S)-3-(4-((3-chloro-4-(((Z)-2-(methyleneamino)but-2-en-1-yl)oxy)phenyl)amino)-7H-pyrrolo[2,3-d]pyrimidin-5-yl)-8-azabicyclo[3.2.1]octan-8-yl)prop-2-en-1-one (2.2 mg, 5.16% yield, 99.9% ee) as a white solid. ¹H NMR (400 MHz, Methanol-d₄) δ 8.56 (d, J=6.0 Hz, 1H), 8.20 (s, 1H), 7.90-7.88 (m, 1H), 7.77 (d, J=2.8 Hz, 1H), 7.72 (d, J=8.4 Hz, 1H), 7.42-7.37 (m, 2H), 7.14 (d, J=8.8 Hz, 1H), 7.01 (s, 1H), 6.71 (dd, J=10.4, 17.2 Hz, 1H), 6.28 (dd, J=2.0, 16.8 Hz, 1H), 5.74 (dd, J=2.0, 10.4 Hz, 1H), 5.27 (s, 2H), 4.58-4.54 (m, 2H), 3.84-3.71 (m, 1H), 2.17-2.07 (m, 2H), 2.08-1.99 (m, 4H), 1.92-1.85 (m, 1H), 1.86-1.67 (m, 1H).

### Example 35: Preparation of N-(4-(4-((5-(1-acryloylpiperidin-4-yl)-7H-pyrrolo[2,3-d]pyrimidin-4-yl)amino)-3-fluorophenoxy)pyridin-2-yl)cyclobutanecarboxamide (Compound 231)

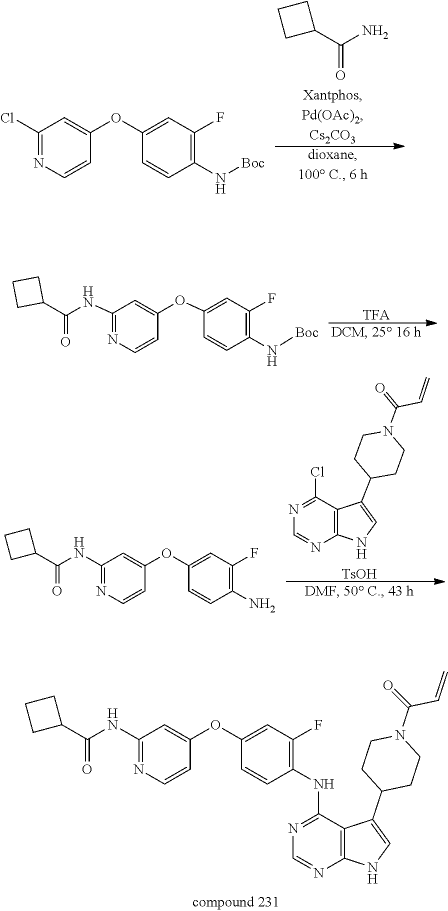 [SMILES: NC(=O)C1CCC1 ; CC(C)(C)OC(=O)Nc1ccc(Oc2ccnc(Cl)c2)cc1F ; CC(C)(C)OC(=O)Nc1ccc(Oc2ccnc(NC(=O)C3CCC3)c2)cc1F ; Nc1ccc(Oc2ccnc(NC(=O)C3CCC3)c2)cc1F ; C=CC(=O)N1CCC(c2c[nH]c3ncnc(Cl)c23)CC1 ; C=CC(=O)N1CCC(c2c[nH]c3ncnc(Nc4ccc(Oc5ccnc(NC(=O)C6CCC6)c5)cc4F)c23)CC1]

### Step 1: Preparation of tert-butyl (4-((2-(cyclobutanecarboxamido)pyridin-4-yl)oxy)-2-fluorophenyl)carbamate

To a solution of cyclobutanecarboxamide (292.63 mg, 2.95 mmol) in dioxane (5 mL) was added tert-butyl (4-((2-chloropyridin-4-yl)oxy)-2-fluorophenyl)carbamate (500 mg, 1.48 mmol), Cs₂CO₃ (1.44 g, 4.43 mmol), Xantphos (85.40 mg, 147.60 μmol) and Pd(OAc)₂ (33.14 mg, 147.60 μmol). The mixture was stirred at 100° C. for 6 h under N₂. LCMS showed 50% peak with desired mass. After cooling to room temperature, the reaction mixture was poured into water (50 mL), extracted with ethyl acetate (50 mL×3). The combined organic layers were washed with brine (50 mL×2), dried over Na₂SO₄, filtered, and concentrated under reduced pressure to give residue. The residue was purified by column chromatography (SiO₂, petroleum ether/ethyl acetate=3/1) to afford tert-butyl (4-((2-(cyclobutanecarboxamido)pyridin-4-yl)oxy)-2-fluorophenyl)carbamate (256 mg, 43.21% yield) as a white solid.

### Step 2: Preparation of N-(4-(4-amino-3-fluorophenoxy)pyridin-2-yl)cyclobutanecarboxamide

To a solution of tert-butyl (4-((2-(cyclobutanecarboxamido)pyridin-4-yl)oxy)-2-fluorophenyl)carbamate (256 mg, 637.72 μmol) in DCM (2 mL) was added TFA (3.08 g, 27.01 mmol). The mixture was stirred at 25° C. for 16 h. LCMS showed 95% peak with desired mass. The reaction mixture was concentrated under reduced pressure to afford N-(4-(4-amino-3-fluorophenoxy)pyridin-2-yl)cyclobutanecarboxamide (190 mg, TFA salt, 71.73% yield) as red oil.

### Step 3: Preparation of N-(4-(4-((5-(1-acryloylpiperidin-4-yl)-7H-pyrrolo[2,3-d]pyrimidin-4-yl)amino)-3-fluorophenoxy)pyridin-2-yl)cyclobutanecarboxamide

To a solution of N-(4-(4-amino-3-fluorophenoxy)pyridin-2-yl)cyclobutanecarboxamide (190 mg, TFA salt, 457.46 μmol) in DMF (1 mL) was added 1-(4-(4-chloro-7H-pyrrolo[2,3-d]pyrimidin-5-yl)piperidin-1-yl)prop-2-en-1-one (90 mg, 309.55 μmol) and TsOH (79.96 mg, 464.32 μmol). The mixture was stirred at 50° C. for 43 h. LCMS showed 13% peak with desired mass. After cooling to room temperature, the reaction mixture was concentrated under reduced pressure to give a residue. The residue was purified by prep-HPLC (FA conditions) to afford N-(4-(4-((5-(1-acryloylpiperidin-4-yl)-7H-pyrrolo[2,3-d]pyrimidin-4-yl)amino)-3-fluorophenoxy)pyridin-2-yl)cyclobutanecarboxamide (5.6 mg, 3.05% yield) as a white solid. ¹H NMR (400 MHz, DMSO-d₆) δ 11.63 (s, 1H), 10.39 (s, 1H), 8.22 (d, J=5.6 Hz, 1H), 8.17 (s, 1H), 7.96-7.94 (m, 2H), 7.78 (d, J=2.0 Hz, 1H), 7.28-7.26 (m, 1H), 7.08-7.06 (m, 2H), 6.84-6.76 (m, 1H), 6.75-6.68 (m, 1H), 6.11 (dd, J=2.4, 16.8 Hz, 1H), 5.76 (dd, J=2.4, 10.4 Hz, 1H), 4.68-4.52 (m, 1H), 4.18 (d, J=15.2 Hz, 1H), 3.46-3.21 (m, 1H), 3.28 (s, 1H), 2.86-2.84 (m, 1H), 2.56 (s, 1H), 2.16-2.06 (m, 6H), 1.94-1.85 (m, 1H), 1.81-1.72 (m, 1H), 1.50-1.47 (m, 2H).

### Example 36: Preparation of N-(4-(4-((5-(1-acryloylpiperidin-4-yl)-7H-pyrrolo[2,3-d]pyrimidin-4-yl)amino)-3-fluorophenoxy)pyridin-2-yl)cyclopropanecarboxamide (Compound 232)

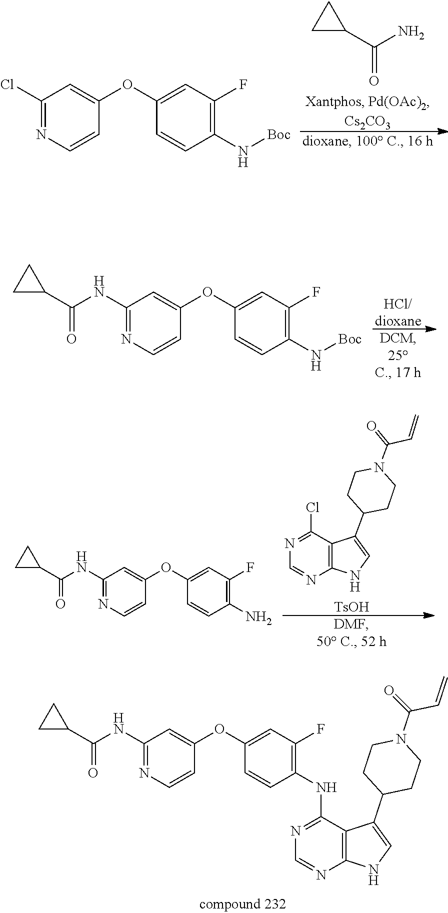

### Step 1: Preparation of tert-butyl (4-((2-(cyclopropanecarboxamido)pyridin-4-yl)oxy)-2-fluorophenyl)carbamate

To a solution of tert-butyl (4-((2-chloropyridin-4-yl)oxy)-2-fluorophenyl)carbamate (500 mg, 1.48 mmol), cyclopropanecarboxamide (251.22 mg, 2.95 mmol) in dioxane (5 mL) was added Xantphos (85.40 mg, 147.60 umol), Pd(OAc)₂ (33.14 mg, 147.60 umol), Cs₂CO₃ (1.44 g, 4.43 mmol). The mixture was stirred at 100° C. for 16 h under N₂. LCMS showed 63% peak with desired mass. After cooling to room temperature, the reaction mixture was diluted with H₂O (5 mL), extracted with ethyl acetate (15 mL×3). The combined organic layers were washed with brine (30 mL), dried over Na₂SO₄, filtered and concentrated under reduced pressure to give a residue. The residue was purified by column chromatography (SiO₂, Ethyl acetate) to afford tert-butyl (4-((2-(cyclopropanecarboxamido)pyridin-4-yl)oxy)-2-fluorophenyl)carbamate (390 mg, 68.21% yield) as a yellow solid.

### Step 2: Preparation of N-(4-(4-amino-3-fluorophenoxy)pyridin-2-yl)cyclopropanecarboxamide

To a solution of tert-butyl (4-((2-(cyclopropanecarboxamido)pyridin-4-yl)oxy)-2-fluorophenyl)carbamate (100 mg, 258.13 umol) in DCM (1 mL) was added HCl/dioxane (4 M, 0.5 mL). The mixture was stirred at 25° C. for 17 h. LCMS showed 86% peak with desired mass. The reaction mixture was concentrated under reduced pressure to afford N-(4-(4-amino-3-fluorophenoxy)pyridin-2-yl)cyclopropanecarboxamide (100 mg, crude) as a light yellow solid.

### Step 3: Preparation of N-(4-((5-(1-acryloylpiperidin-4-yl)-7H-pyrrolo[2,3-d]pyrimidin-4-yl)amino)-3-fluorophenoxy)pyridin-2-yl)cyclopropanecarboxamide

To a solution of N-(4-(4-amino-3-fluorophenoxy)pyridin-2-yl)cyclopropanecarboxamide (100 mg, 348.08 umol) and 1-(4-(4-chloro-7H-pyrrolo[2,3-d]pyrimidin-5-yl)piperidin-1-yl)prop-2-en-1-one (80.96 mg, 278.47 umol) in DMF (1 mL) was added TsOH (89.91 mg, 522.12 umol). The mixture was stirred at 50° C. for 52 h. LCMS showed 39% peak with desired mass. After cooling to room temperature, the reaction mixture was purified by prep-HPLC (TFA conditions) to afford N-(4-(4-((5-(1-acryloylpiperidin-4-yl)-7H-pyrrolo[2,3-d]pyrimidin-4-yl)amino)-3-fluorophenoxy)pyridin-2-yl)cyclopropanecarboxamide (32.4 mg, 16.48% yield, 2TFA salts) as a white solid. ¹H NMR (400 MHz, DMSO-d₆) δ 12.06 (s, 1H), 11.00 (s, 1H), 8.77-8.35 (m, 1H), 8.27-8.23 (m, 2H), 7.92-7.87 (m, 1H), 7.71 (d, J=2.4 Hz, 1H), 7.24-7.18 (m, 1H), 7.15-7.13 (m, 1H), 7.12-7.12 (m, 1H), 6.88-6.84 (m, 1H), 6.82-6.79 (m, 1H), 6.11 (dd, J=2.4, 16.8 Hz, 1H), 5.67 (dd, J=2.4, 10.4 Hz, 1H), 4.61-4.57 (m, 1H), 4.19-4.17 (m, 1H), 3.50-3.47 (m, 1H), 3.32-3.25 (m, 1H), 2.89-2.77 (m, 1H), 2.09-2.04 (m, 2H), 2.00-1.97 (m, 1H), 1.51-1.47 (m, 2H), 0.81-0.77 (m, 4H).

### Example 36: Cellular Proliferation Assays

Generation of HCC827-SVD, HCC827-ASV, and HCC827-YVMA cell lines. The HCC827-SVD cell line was generated by first transducing HCC827 cells (ATCC) with lentiviral particles expressing codon-optimized human EGFR exon 20 insertion mutant D770_N771insSVD, and then selecting under 5 pg/mL puromycin treatment for a week. The resulting cells were further transduced with lentiviral particles carrying EGFR sgRNA and CAS9 to knockout the endogenous alleles of EGER and then selected under 500 pg/mL hygromycin B treatment for two weeks. The limiting dilution approach was used to subsequently generate the single clone of the HCC827-SVD stable cell line. Endogenous EGFR knockout was confirmed by Sanger sequencing of cell genomic DNA, and expression of exogenous mutant EGFR was verified by Western blot analysis.

HCC827-ASV and HCC827-YVMA cell lines were generated in a similar approach as HCC827-SVD, except that HCC827 cells (ATCC) were first transduced with lentiviral particles expressing codon-optimized human EGFR exon 20 insertion mutant V769_D770insASV and human ERBB2 exon20 insertion mutant A775_G776ins YVMA, respectively.

2. Cell Growth Inhibition for HCC827, HCC827-SVD, HCC827-ASV, HCC827-YVMA, and A431.

HCC827(ATCC), HCC827-SVD, HCC827-ASV, and HCC827-YVMA cells were seeded in 96-well plates at 5000 cells/well in 90 μL of RPMI growth medium containing 10% FBS and 1% Penicillin Streptomycin. A431 (ATCC) cells were seeded in 96-well plates at 5000 cells/well in 90 μL of DMEM growth medium containing 10% FBS and 1% Penicillin Streptomycin. Cells were incubated at 37° C. overnight and then administered with 10 μL 10× test compounds prepared in the growth medium at various concentrations. After administration of the compounds, cells were incubated at 37° C. for 3 days. Before the CellTiter-Glo assay, the plates were equilibrated at room temperature for approximately 30 minutes. 100 ul of CellTiter-Glo reagent (Promega) was added to each well. The plates were then incubated at room temperature for 10 minutes and luminescence was recorded by the EnSight® multimode plate reader (PerkinElmer). Table 7 listed selected compounds in cellular growth assay.

TABLE 7
Cell Growth Inhibition of the Compounds of the Present Disclosure

| | Cell growth inhibition, GI50 (nM) | | | | |
|---|---|---|---|---|---|
| Compound | HCC827- | HCC827- | HCC827- | | |
| No. | ASV | YVMA | SVD | A431 | HCC827 |
| 35 | | A | A | | |
| 36 | | A | A | | |
| 37 | | A | A | | |
| 38 | | B | B | | |
| 39 | | A | A | | |
| 40 | | A | A | | |
| 41 | | C | B | | |
| 42 | | C | C | | |
| 43 | | B | B | | |
| 44 | | C | C | | |
| 45 | | B | B | | |
| 46 | | B | B | | |
| 47 | | B | A | | |
| 48 | | B | B | | |
| 49 | | A | B | | |
| 50 | | B | C | | |
| 51 | | C | C | | |
| 52 | | A | A | | |
| 53 | | C | C | | |
| 54 | | A | A | | |
| 55 | | A | A | | |
| 56 | | C | C | | |
| 57 | | C | C | | |
| 58 | | B | C | | |
| 59 | | B | D | | |
| 60 | | C | B | | |
| 61 | | C | D | | |
| 62 | | B | C | | |
| 63 | | C | C | | |
| 64 | | C | D | | |
| 65 | | C | C | | |
| 66 | | B | C | | |
| 67 | | C | C | | |
| 68 | | A | A | | |
| 69 | | A | A | | |
| 70 | | B | A | | |
| 71 | | A | A | | |
| 72 | | B | C | | |
| 73 | | A | A | | |
| 74 | | A | A | | |
| 75 | | A | A | | |
| 76 | | A | A | | |
| 77 | | A | A | | |
| 78 | | B | C | | |
| 79 | | C | C | | |
| 80 | | A | A | | |
| 81 | | B | B | | |
| 82 | | C | D | | |
| 83 | | B | C | | |
| 84 | | B | A | | |
| 85 | | B | B | | |
| 86 | | A | A | | |
| 87 | | B | B | | |
| 88 | | B | B | | |
| 89 | | B | B | | |
| 90 | A | A | B | C | B |
| 91 | | A | C | | |
| 92 | | B | B | | B |
| 93 | | B | B | | B |
| 94 | | B | B | | B |
| 95 | | A | A | | A |
| 96 | | C | D | | C |
| 97 | | C | C | | C |
| 98 | | A | A | | A |
| 99 | | A | B | | B |
| 100 | | A | A | | A |
| 101 | | B | B | | B |
| 102 | | A | B | | B |
| 103 | | B | B | | B |
| 104 | | A | B | | C |
| 105 | | A | A | | A |
| 106 | | A | A | | A |
| 107 | | B | C | | D |
| 108 | | D | D | | C |
| 109 | | A | B | | A |
| 110 | | A | A | C | A |
| 111 | | A | A | B | A |
| 112 | | B | C | | B |
| 113 | | A | A | A | A |
| 114 | | A | A | | A |
| 115 | | B | B | | B |
| 116 | | A | A | B | A |
| 117 | | A | A | | A |
| 118 | | A | A | | A |
| 119 | | A | A | B | A |
| 120 | | A | A | | A |
| 121 | | A | A | | A |
| 122 | | C | C | | D |
| 124 | | B | C | | C |
| 125 | | C | C | | D |
| 126 | | A | A | | A |
| 127 | | A | A | | B |
| 128 | | A | A | | A |
| 129 | | C | C | | |
| 130 | | B | B | | |
| 131 | | B | B | | D |
| 132 | | B | C | | D |
| 133 | | B | B | | D |
| 134 | | B | B | | B |
| 135 | A | A | A | C | D |
| 136 | A | A | B | D | C |
| 137 | | A | A | D | B |
| 138 | | C | B | D | D |
| 139 | | D | B | D | D |
| 140 | | B | B | D | C |
| 141 | | B | B | D | D |
| 142 | | A | A | D | D |
| 143 | | A | A | D | A |
| 144 | A | A | D | D | B |
| 145 | | C | C | D | D |
| 146 | | C | C | D | D |
| 147 | | B | B | D | D |
| 148 | | C | B | D | D |
| 149 | | B | B | D | D |
| 150 | | B | B | D | C |
| 151 | | B | C | | D |
| 152 | C | C | C | D | D |
| 153 | | A | A | A | A |
| 154 | A | A | A | C | A |
| 155 | C | B | B | D | D |
| 156 | A | A | A | A | A |
| 157 | B | B | B | D | C |
| 158 | C | B | D | D | D |
| 159 | C | C | C | D | D |
| 160 | A | A | A | A | A |
| 161 | | B | C | | |
| 162 | | C | D | | |
| 163 | | A | A | D | A |
| 164 | C | B | C | C | C |
| 165 | | B | B | D | C |
| 166 | A | A | A | A | A |
| 167 | B | B | B | D | D |
| 168 | | D | D | D | C |
| 169 | | D | D | D | D |
| 170 | | B | B | | C |
| 171 | | A | B | D | C |
| 172 | | B | B | D | C |
| 173 | A | A | B | D | C |
| 174 | | C | C | D | D |
| 175 | B | B | B | D | D |
| 176 | | C | C | D | D |
| 177 | C | B | C | D | D |
| 178 | | B | C | D | D |
| 179 | | C | C | D | D |
| 180 | B | A | B | D | C |
| 181 | | B | B | D | D |
| 182 | | B | B | D | D |
| 183 | | C | C | D | C |
| 184 | | C | D | D | D |
| 185 | | B | B | | |
| 186 | | B | C | D | C |
| 187 | | C | C | D | C |
| 188 | | B | B | D | C |
| 189 | | C | C | | D |
| 190 | | B | B | D | C |
| 191 | | C | C | D | C |
| 192 | | B | B | | |
| 193 | | B | B | | D |
| 194 | | C | C | | D |
| 195 | | B | A | D | A |
| 196 | | C | C | | |
| 197 | D | C | D | D | D |
| 198 | A | A | A | A | A |
| 199 | A | A | A | D | B |
| 200 | | B | B | D | C |
| 201 | D | C | D | D | D |
| 202 | C | B | B | D | D |
| 203 | | B | C | D | D |
| 204 | | B | B | D | D |
| 205 | | B | C | D | D |
| 206 | | B | C | D | D |
| 207 | | B | C | D | D |
| 216 | A | A | A | | |
| 217 | A | A | A | | |
| 218 | A | A | A | | |
| 219 | A | A | A | | |
| 220 | A | A | A | | |
| 221 | A | A | A | | |
| 222 | A | A | A | | |
| 223 | A | A | A | | |
| 224 | A | A | A | | |
| 225 | | A | | | |
| 226 | A | A | A | | |
| 227 | A | A | A | | |
| 228 | A | A | A | | |
| 229 | A | A | A | | |
| 230 | A | A | A | | |
| 231 | B | B | B | | |
| 232 | A | A | A | D | |

Cell growth inhibition data (GI50):
A: <=50 nmol;
B: 50 to 200 nmol (including 200 nmol);
C: 200 to 1000 nmol (including 1000 nmol);
D: >1000 nmol.

1. A compound of Formula (1) or a tautomer, stereoisomer or a mixture of stereoisomers, or a pharmaceutically acceptable salt, or hydrate, or deuterated derivative thereof: 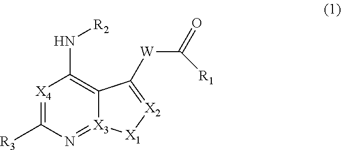
wherein:
X₁ is chosen from NH, N—CH₃, S, and CH;
X₂ is chosen from CH, N, C—CH₃, and N-methylpiperazine-4-phenyl;
X₃ is chosen from C and N;
X₄ is chosen from N, C—CN, and C—F;
R₃ is chosen from H and C₁-C₅ alkyl;
R₁ is chosen from 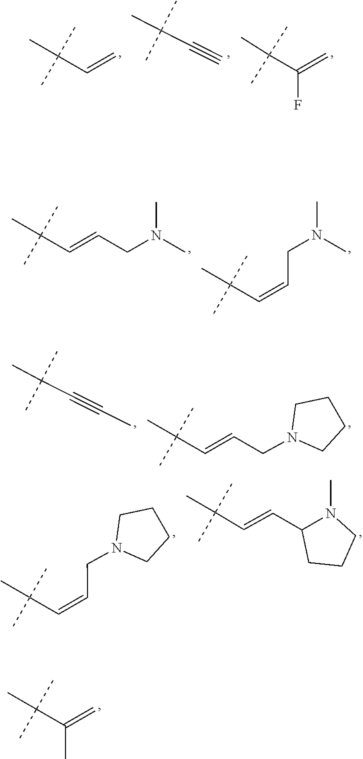
and C₁-C₁₀ alkyl;
W is chosen from alkylamino group, azetidine, methylazetidine, azetidine-3-amine, piperidine, tetrahydropyridine, pyrrolidine, 3-aminopyrrolidine, cyclobutylamine, cyclopentylamine, azepane, bicyclic amine, bridge cyclic amine, a spirocyclic amine, and an aryl amine, each of which is optionally substituted with halogen, hydroxyl, C₁-C₅ alkyl, and C₁-C₅ alkoxy; and
R₂ is chosen from 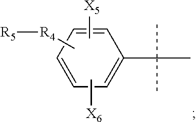
wherein:
X₅ and X₆ are each independently a halogen or a cyano group,
R₄ is chosen from a C₁-C₃ alkyl, a C₁-C₃ alkoxy, 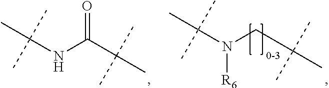
and O, and
R₅ is chosen from optionally substituted aryls and optionally substituted heteroaryls;
wherein R₆ is H or CH₃.

2. The compound according to claim 1, or a pharmaceutically acceptable salt thereof, having a structure according to Formula (2):

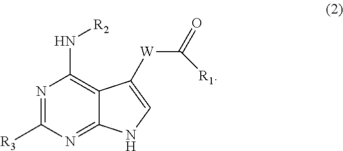

3. The compound according to claim 1, or a pharmaceutically acceptable salt thereof, having a structure according to Formula (3):

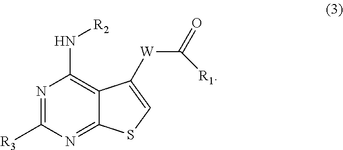

4. The compound according to claim 1, or a pharmaceutically acceptable salt thereof, having a structure according to Formula (4):

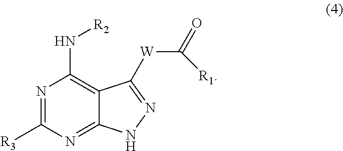

5. The compound according to claim 1, or a pharmaceutically acceptable salt thereof, having a structure according to Formula (5):

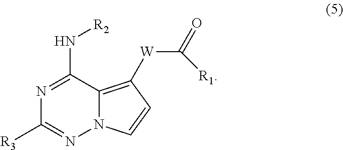

6. The compound according to claim 1, wherein W is chosen from

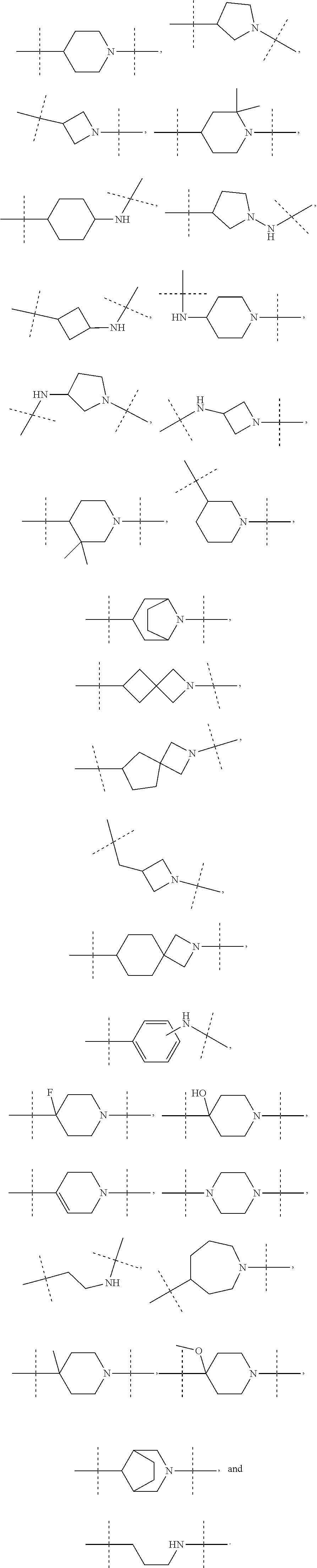

7. The compound according to claim 1, wherein R₂ is chosen from

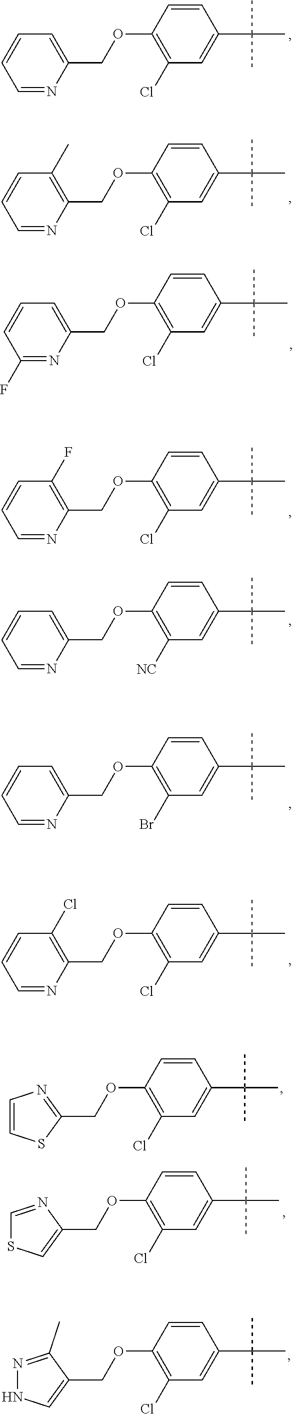

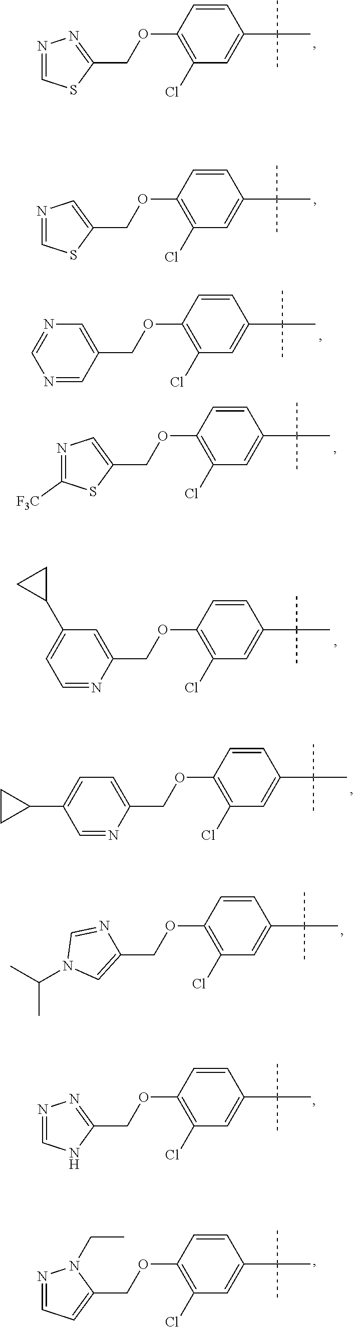

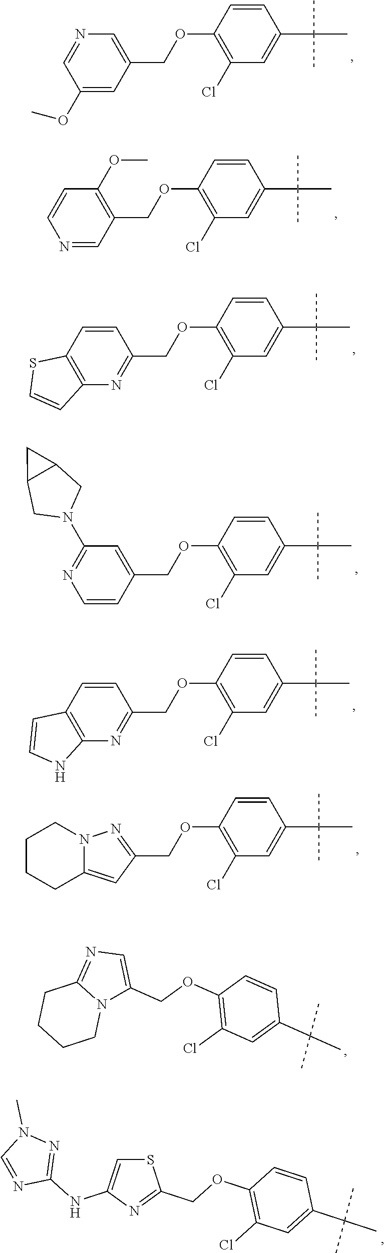

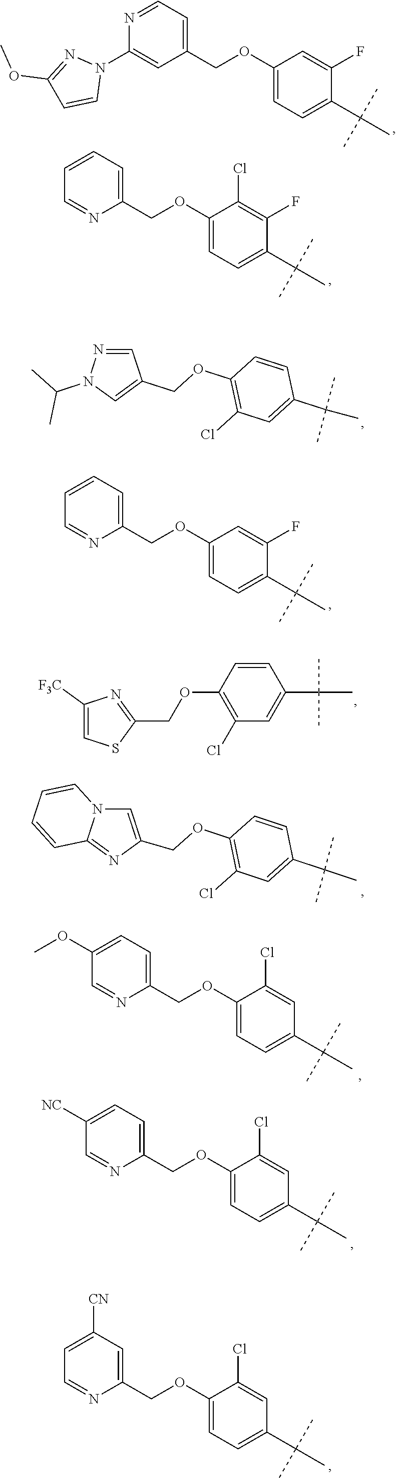

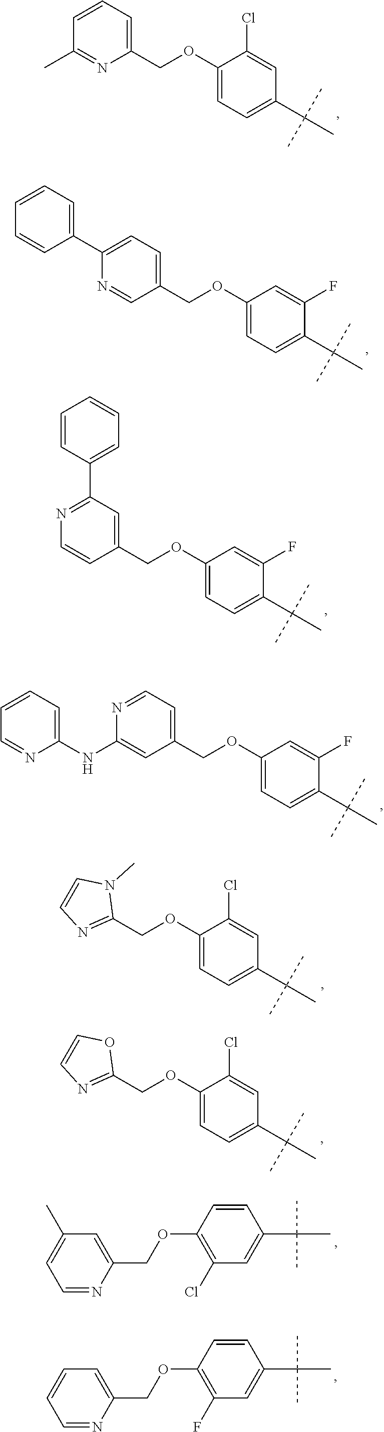

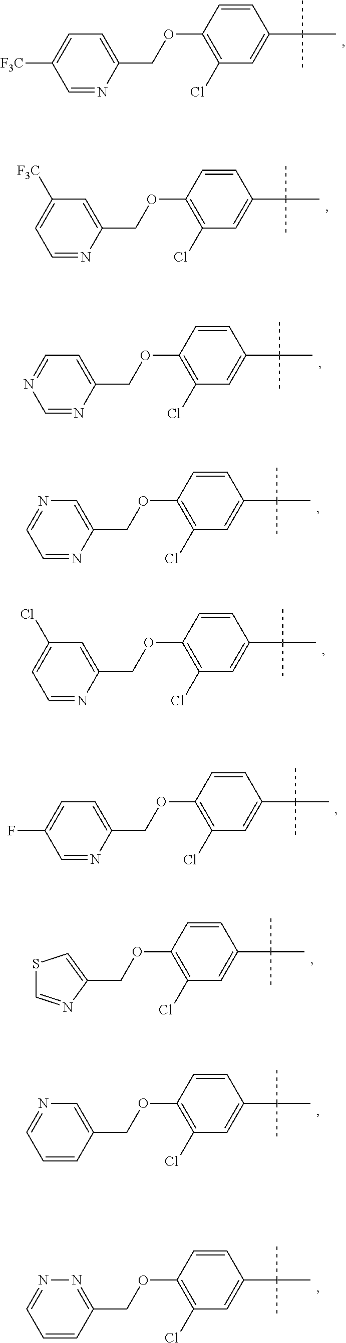

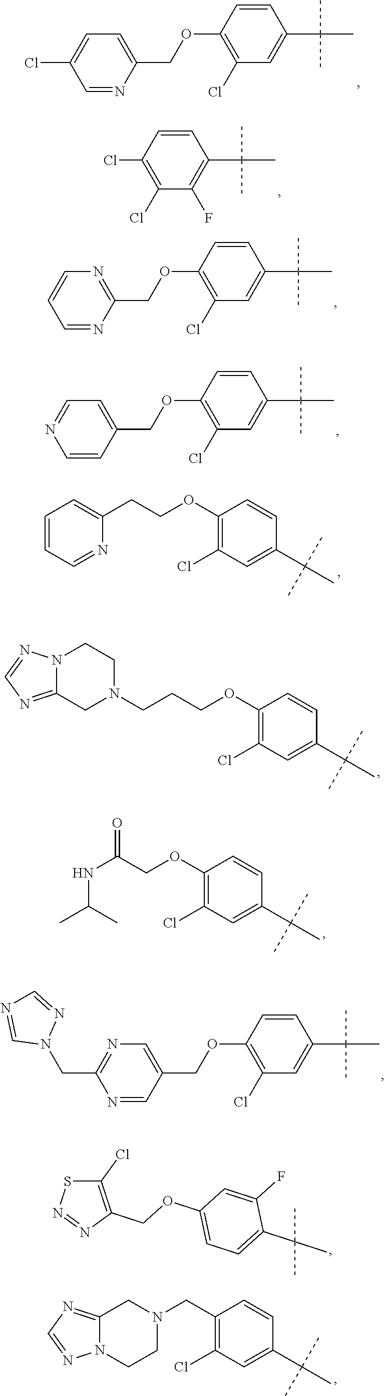

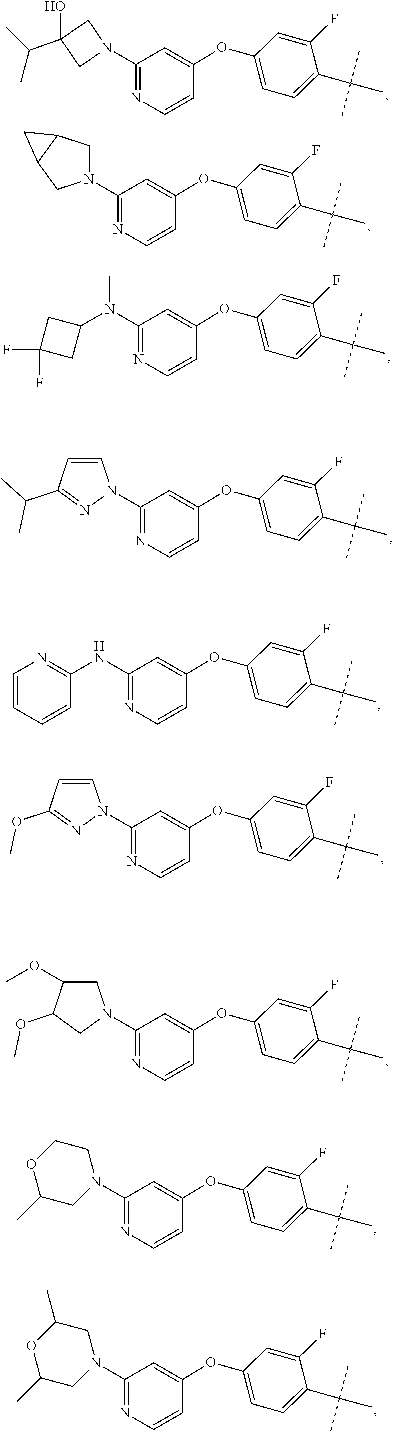

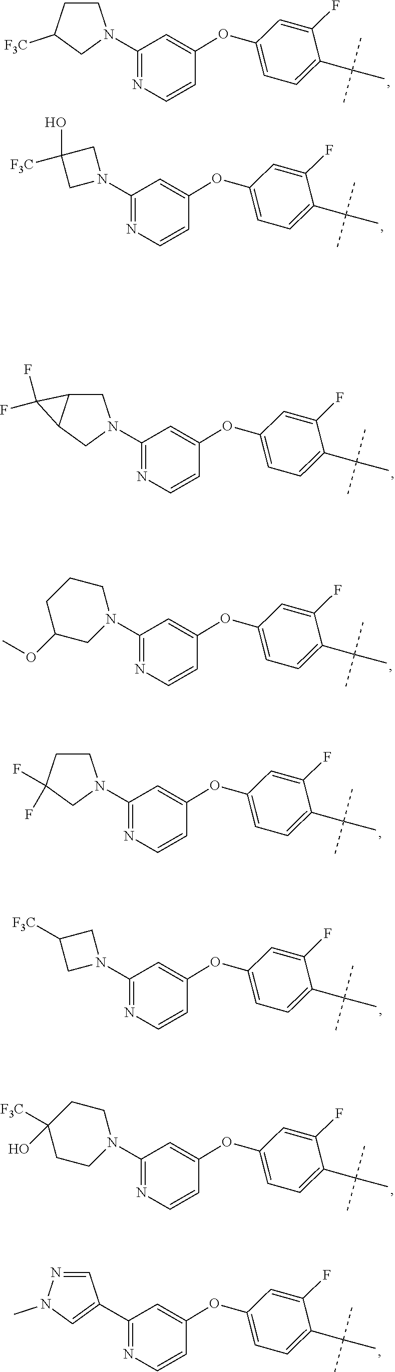

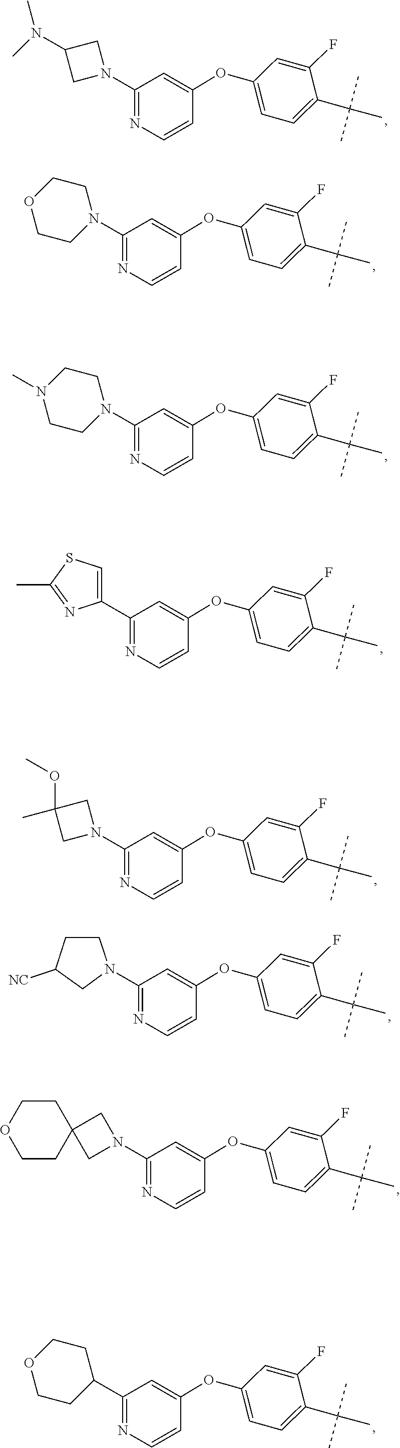

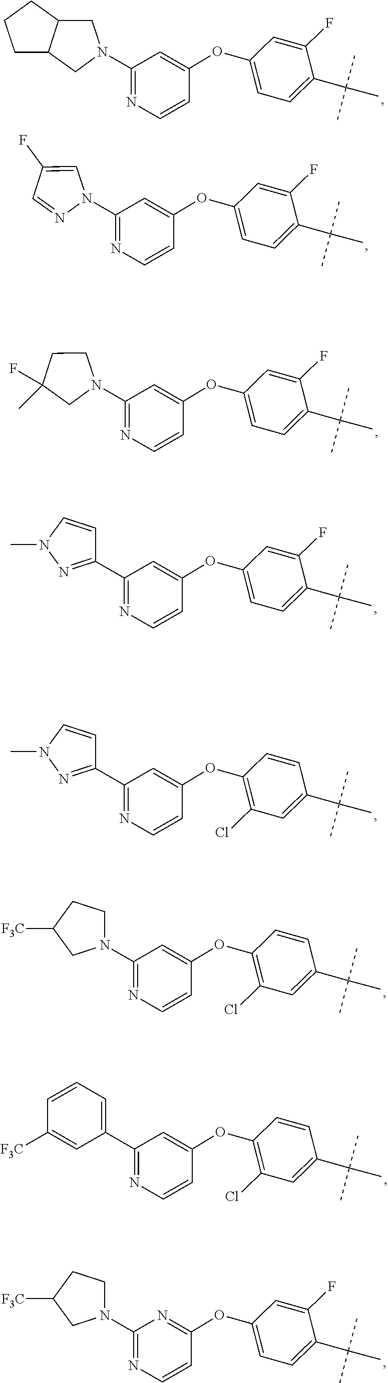

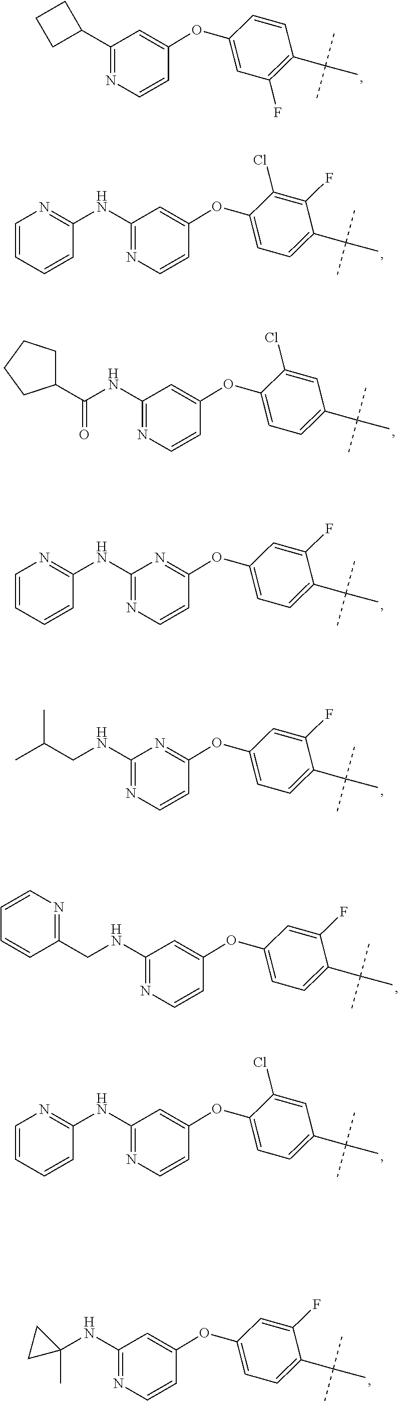

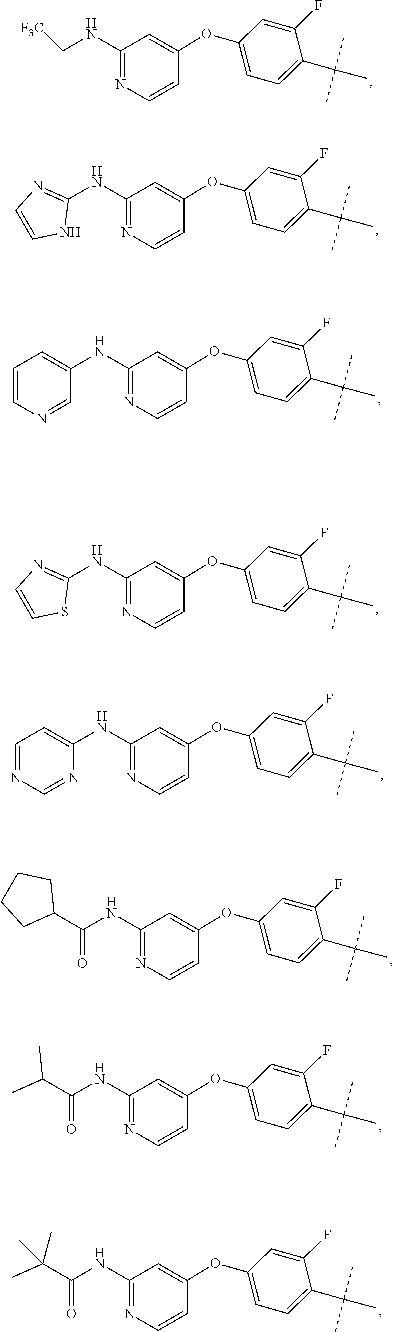

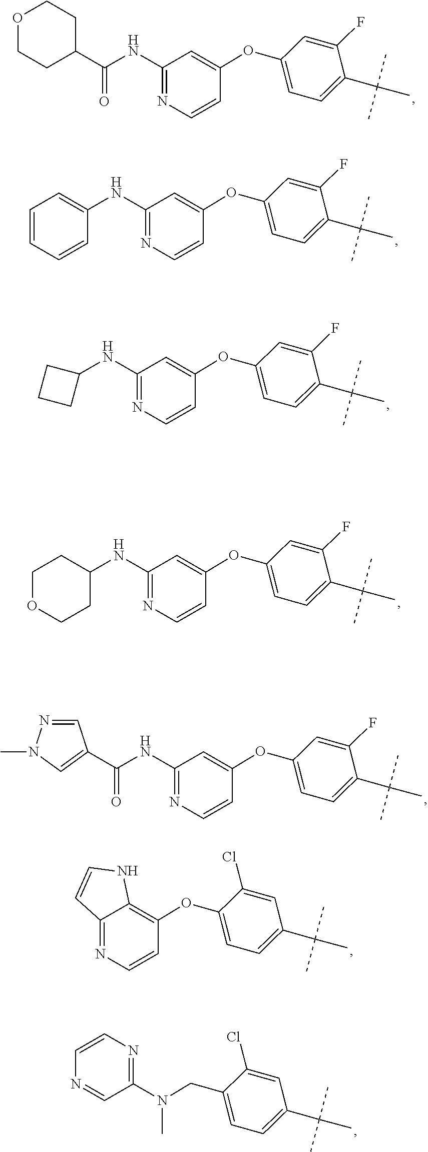

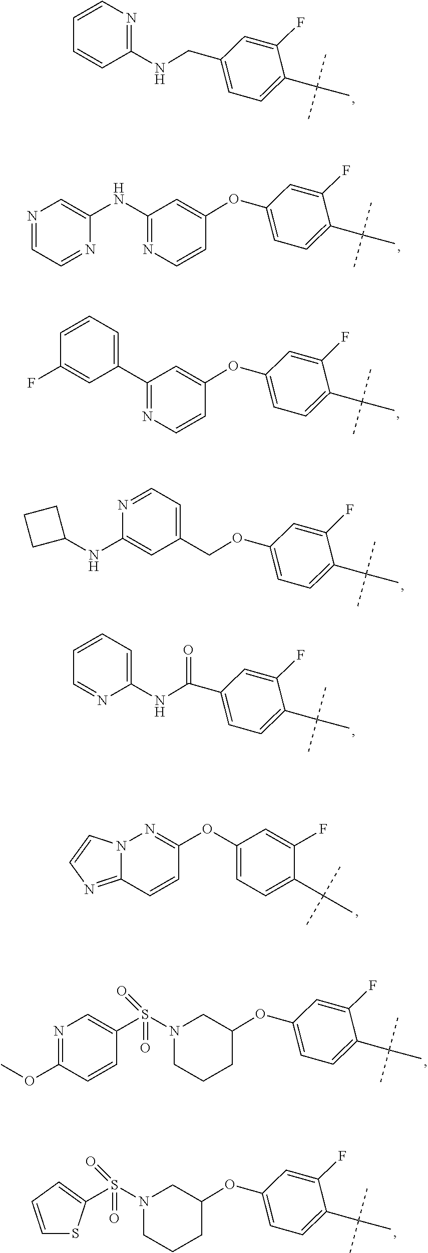

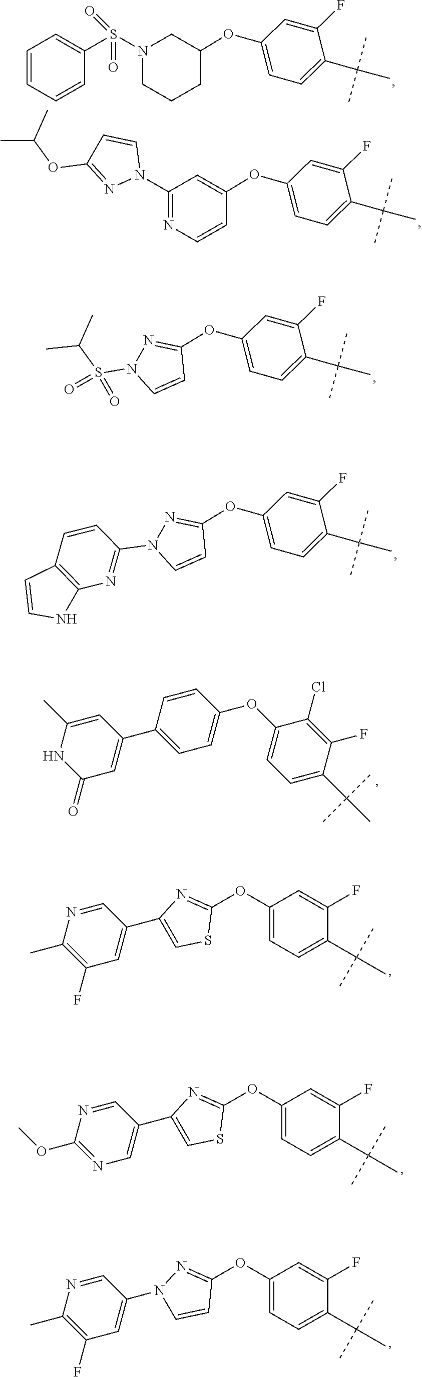

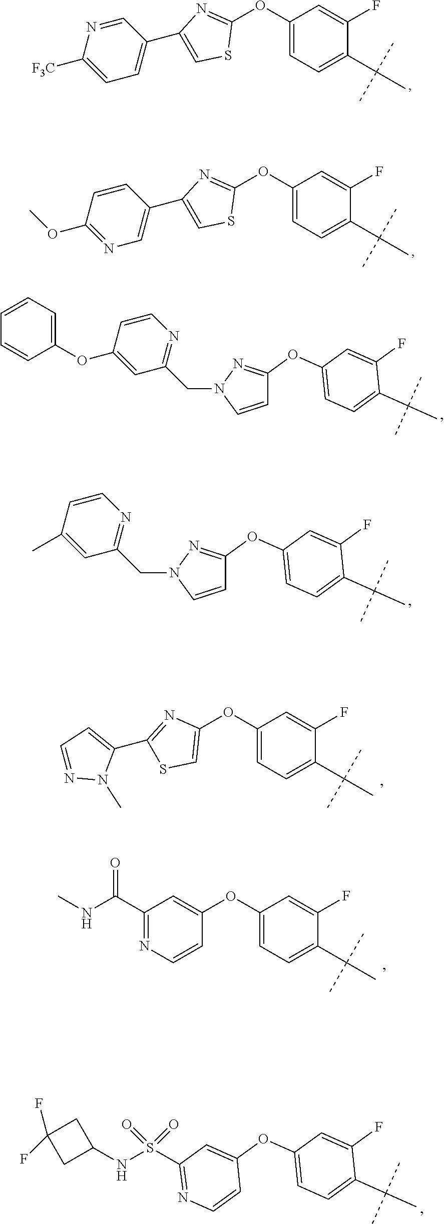

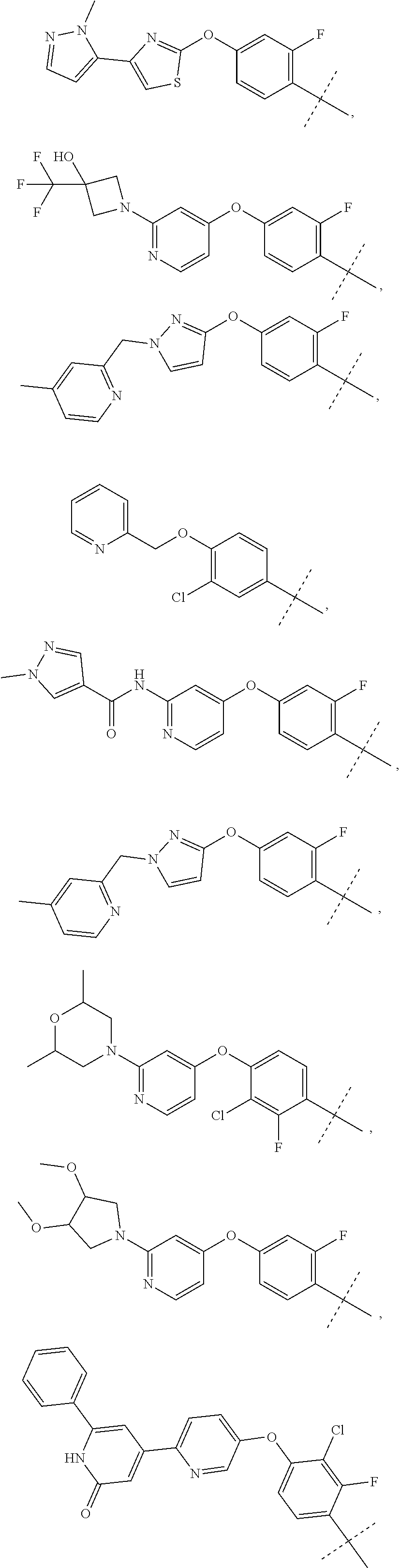

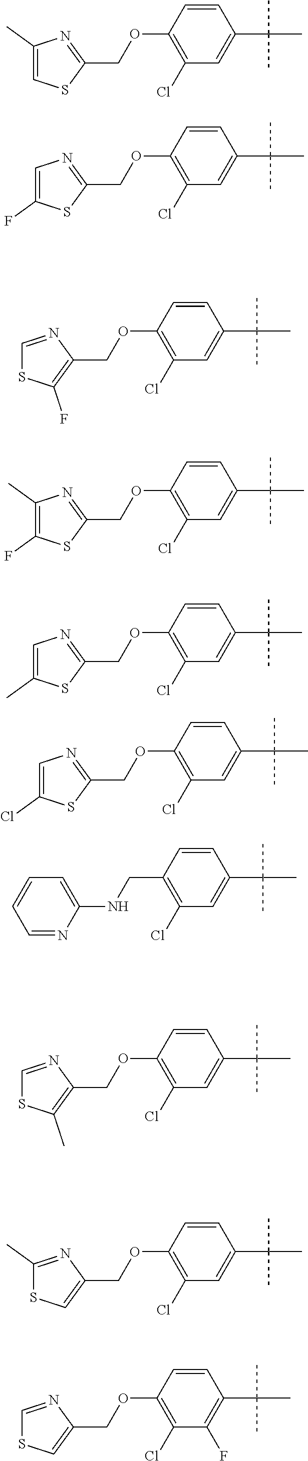

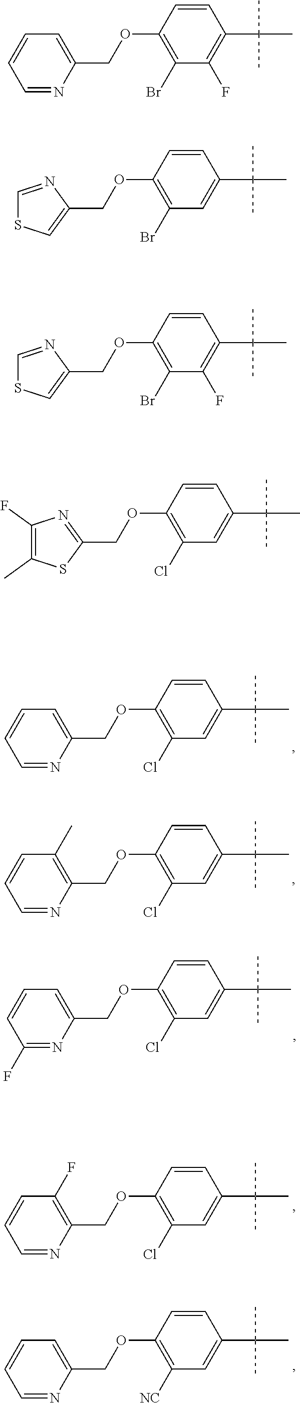

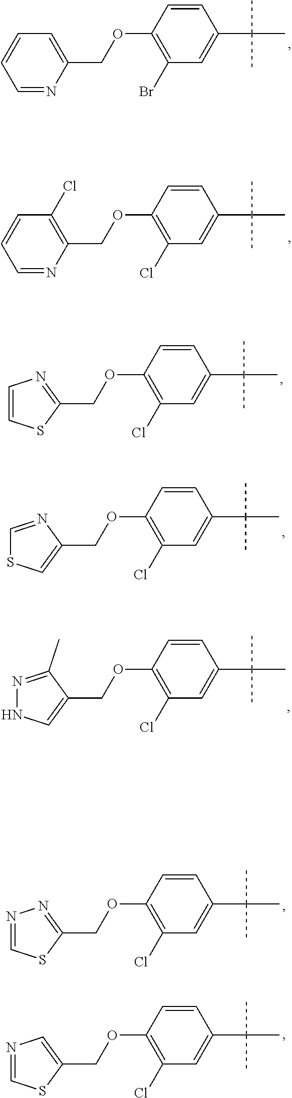

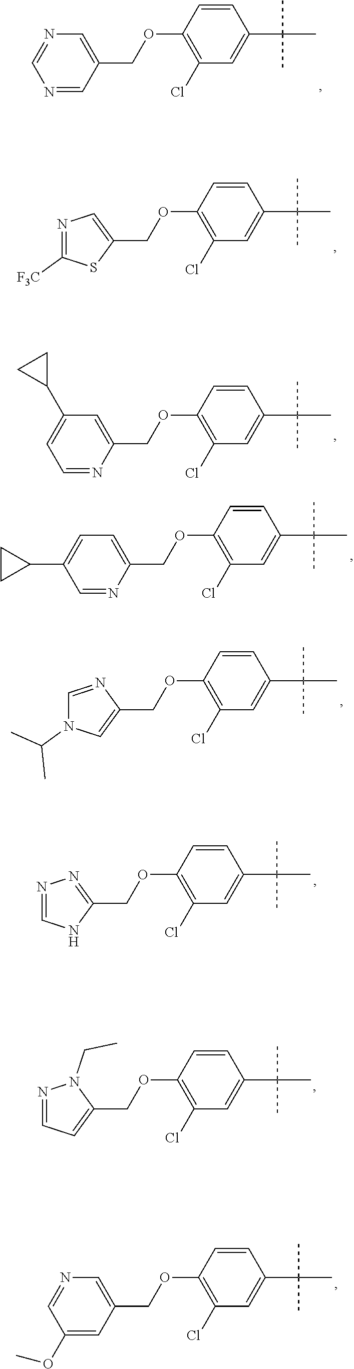

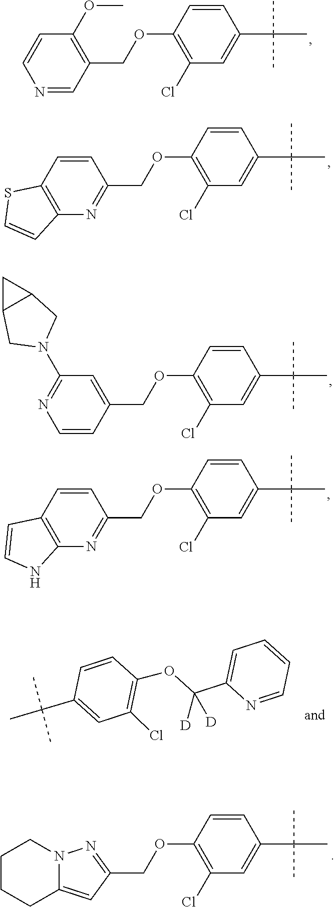

8-14. (canceled)

15. The compound according to claim 1, wherein the compound is chosen from:
(8-syn)-8-(4-((3-chloro-4-(pyridin-2-ylmethoxy)phenyl)amino)-7H-pyrrolo[2,3-d]pyrimidin-5-yl)-3-azabicyclo[3.2.1]octan-3-yl)prop-2-en-1-one;
(8-anti)-8-(4-((3-chloro-4-(pyridin-2-ylmethoxy)phenyl)amino)-7H-pyrrolo[2,3-d]pyrimidin-5-yl)-3-azabicyclo[3.2.1]octan-3-yl)prop-2-en-1-one;
1-(4-(4-((3-chloro-4-(pyridin-2-ylmethoxy)phenyl)amino)-5-fluoro-1H-pyrrolo[2,3-b]pyridin-3-yl)piperidin-1-yl)prop-2-en-1-one;
1-(4-(4-((3-chloro-4-(pyridin-2-ylmethoxy)phenyl)amino)-1H-pyrrolo[2,3-b]pyridin-3-yl)piperidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)-4-hydroxypiperidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)-4-methylpiperidin-1-yl)prop-2-en-1-one;
1-(4-(4-((3-chloro-4-(pyridin-2-ylmethoxy-d2)phenyl)amino)-7H-pyrrolo[2,3-d]pyrimidin-5-yl)piperidin-1-yl)prop-2-en-1-one;
1-(4-(4-((3-chloro-4-(pyridin-2-ylmethoxy)phenyl)amino)-7-methyl-7H-pyrrolo[2,3-d]pyrimidin-5-yl)piperidin-1-yl)prop-2-en-1-one;
N-(2-(4-((3-chloro-4-(pyridin-2-ylmethoxy)phenyl)amino)-7H-pyrrolo[2,3-d]pyrimidin-5-yl)ethyl)acrylamide;
N-((1,3-trans)-3-(4-((3-chloro-4-(pyridin-2-ylmethoxy)phenyl)amino)-7H-pyrrolo[2,3-d]pyrimidin-5-yl)cyclobutyl)acrylamide;
N-((1,3-cis)-3-(4-((3-chloro-4-(pyridin-2-ylmethoxy)phenyl)amino)-7H-pyrrolo[2,3-d]pyrimidin-5-yl)cyclobutyl)acrylamide;
1-(3-((4-((3-chloro-4-(pyridin-2-ylmethoxy)phenyl)amino)-7H-pyrrolo[2,3-d]pyrimidin-5-yl)methyl)azetidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)-4-fluoropiperidin-1-yl)prop-2-en-1-one;
3-(1-acryloylpiperidin-4-yl)-4-((3-chloro-4-(pyridin-2-ylmethoxy)phenyl)amino)-1H-pyrrolo[2,3-b]pyridine-5-carbonitrile;
(E)-1-(4-(4-((3-chloro-4-(pyridin-2-ylmethoxy)phenyl)amino)-7H-pyrrolo[2,3-d]pyrimidin-5-yl)piperidin-1-yl)-4-(dimethylamino)but-2-en-1-one;
1-(4-(4-((3-chloro-4-(pyridin-2-ylmethoxy)phenyl)amino)-2-methyl-7H-pyrrolo[2,3-d]pyrimidin-5-yl)piperidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)piperidin-1-yl)-2-fluoroprop-2-en-1-one;
N-((1,4-cis)-4-(4-((3-chloro-4-(pyridin-2-ylmethoxy)phenyl)amino)-7H-pyrrolo[2,3-d]pyrimidin-5-yl)cyclohexyl)acrylamide;
N-((1,4-trans)-4-(4-((3-chloro-4-(pyridin-2-ylmethoxy)phenyl)amino)-7H-pyrrolo[2,3-d]pyrimidin-5-yl)cyclohexyl)acrylamide;
1-(6-(4-((3-chloro-4-(pyridin-2-ylmethoxy)phenyl)amino)-7H-pyrrolo[2,3-d]pyrimidin-5-yl)-2-azaspiro[3.4]octan-2-yl)prop-2-en-1-one;
1-(6-(4-((3-chloro-4-(pyridin-2-ylmethoxy)phenyl)amino)-7H-pyrrolo[2,3-d]pyrimidin-5-yl)-2-azaspiro[3.3]heptan-2-yl)prop-2-en-1-one;
1-(7-(4-((3-chloro-4-(pyridin-2-ylmethoxy)phenyl)amino)-7H-pyrrolo[2,3-d]pyrimidin-5-yl)-2-azaspiro[3.5]nonan-2-yl)prop-2-en-1-one;
1-(4-(4-((3-chloro-4-(pyridin-2-ylmethoxy)phenyl)amino)-7H-pyrrolo[2,3-d]pyrimidin-5-yl)azepan-1-yl)prop-2-en-1-one;
1-(4-(4-((3-chloro-4-(pyridin-2-ylmethoxy)phenyl)amino)-7H-pyrrolo[2,3-d]pyrimidin-5-yl)-2,2-dimethylpiperidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)-3,3-dimethylpiperidin-1-yl)prop-2-en-1-one;
1-(3-(4-((3-chloro-4-(pyridin-2-ylmethoxy)phenyl)amino)-7H-pyrrolo[2,3-d]pyrimidin-5-yl)pyrrolidin-1-yl)prop-2-en-1-one;
3-(4-((3-chloro-4-(pyridin-2-ylmethoxy)phenyl)amino)-7H-pyrrolo[2,3-d]pyrimidin-5-yl)-3-exo-8-azabicyclo[3.2.1]octan-8-yl)prop-2-en-1-one;
3-(4-((3-chloro-4-(pyridin-2-ylmethoxy)phenyl)amino)-7H-pyrrolo[2,3-d]pyrimidin-5-yl)-3-endo-8-azabicyclo[3.2.1]octan-8-yl)prop-2-en-1-one;
1-(4-(4-((3-chloro-4-((3-methylpyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-(2-(pyridin-2-yl)ethoxy)phenyl)amino)-7H-pyrrolo[2,3-d]pyrimidin-5-yl)piperidin-1-yl)prop-2-en-1-one;
5-((5-(1-acryloylpiperidin-4-yl)-7H-pyrrolo[2,3-d]pyrimidin-4-yl)amino)-2-(pyridin-2-ylmethoxy)benzonitrile;
1-(4-(4-((3-chloro-4-((3-methyl-1H-pyrazol-4-yl)methoxy)phenyl)amino)-7H-pyrrolo[2,3-d]pyrimidin-5-yl)piperidin-1-yl)prop-2-en-1-one;
1-(4-(4-((3-chloro-4-((5,6-dihydro-[1,2,4]triazolo[1,5-a]pyrazin-7(8H)-yl)methyl)phenyl)amino)-7H-pyrrolo[2,3-d]pyrimidin-5-yl)piperidin-1-yl)prop-2-en-1-one;
1-(4-(4-((3-chloro-4-(3-(5,6-dihydro-[1,2,4]triazolo[1,5-a]pyrazin-7(8H)-yl)propoxy)phenyl)amino)-7H-pyrrolo[2,3-d]pyrimidin-5-yl)piperidin-1-yl)prop-2-en-1-one;
1-(4-(4-((3-chloro-4-((3-chloropyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-((6-fluoropyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-((3-fluoropyridin-2-yl)methoxy)phenyl)amino)-7H-pyrrolo[2,3-d]pyrimidin-5-yl)piperidin-1-yl)prop-2-en-1-one;
N-(4-(4-((5-(1-acryloylpiperidin-4-yl)-7H-pyrrolo[2,3-d]pyrimidin-4-yl)amino)-2-chlorophenoxy)pyridin-2-yl)cyclopentanecarboxamide;
1-(4-(4-((3-bromo-4-(pyridin-2-ylmethoxy)phenyl)amino)-7H-pyrrolo[2,3-d]pyrimidin-5-yl)piperidin-1-yl)prop-2-en-1-one;
1-(4-(4-((3-chloro-2-fluoro-4-((2-(pyridin-2-ylamino)pyridin-4-yl)oxy)phenyl)amino)-7H-pyrrolo[2,3-d]pyrimidin-5-yl)piperidin-1-yl)prop-2-en-1-one;
1-(4-(4-((4-((1-(1H-pyrrolo[2,3-b]pyridin-6-yl)-1H-pyrazol-3-yl)oxy)-2-fluorophenyl)amino)-7H-pyrrolo[2,3-d]pyrimidin-5-yl)piperidin-1-yl)prop-2-en-1-one;
1-(4-(4-((2-fluoro-4-((2-((pyridin-2-ylmethyl)amino)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((2-(3-(trifluoromethyl)pyrrolidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(pyridin-2-ylamino)pyrimidin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(isobutylamino)pyrimidin-4-yl)oxy)phenyl)amino)-7H-pyrrolo[2,3-d]pyrimidin-5-yl)piperidin-1-yl)prop-2-en-1-one;
1-(4-(4-((4-((1H-pyrrolo[3,2-b]pyridin-7-yl)oxy)-3-chlorophenyl)amino)-7H-pyrrolo[2,3-d]pyrimidin-5-yl)piperidin-1-yl)prop-2-en-1-one;
1-(4-(4-((4-((2-((1H-1,2,4-triazol-1-yl)methyl)pyrimidin-5-yl)methoxy)-3-chlorophenyl)amino)-7H-pyrrolo[2,3-d]pyrimidin-5-yl)piperidin-1-yl)prop-2-en-1-one;
1-(4-(4-((3-chloro-4-((2-(1-methyl-1H-pyrazol-3-yl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((2-(pyridin-2-ylamino)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(3-(trifluoromethyl)pyrrolidin-1-yl)pyrimidin-4-yl)oxy)phenyl)amino)-7H-pyrrolo[2,3-d]pyrimidin-5-yl)piperidin-1-yl)prop-2-en-1-one;
1-(4-(4-((4-((2-cyclobutylpyridin-4-yl)oxy)-2-fluorophenyl)amino)-7H-pyrrolo[2,3-d]pyrimidin-5-yl)piperidin-1-yl)prop-2-en-1-one;
1-(4-(4-((4-((1,3,4-thiadiazol-2-yl)methoxy)-3-chlorophenyl)amino)-7H-pyrrolo[2,3-d]pyrimidin-5-yl)piperidin-1-yl)prop-2-en-1-one;
1-(4-(4-((2-fluoro-4-((2-(3-(trifluoromethyl)phenyl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((methyl(pyrazin-2-yl)amino)methyl)phenyl)amino)-7H-pyrrolo[2,3-d]pyrimidin-5-yl)piperidin-1-yl)prop-2-en-1-one;
2-(4-((5-(1-acryloylpiperidin-4-yl)-7H-pyrrolo[2,3-d]pyrimidin-4-yl)amino)-2-chlorophenoxy)-N-isopropylacetamide;
1-(4-(4-((2-fluoro-4-((2-((1-methylcyclopropyl)amino)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(3-fluoro-3-methylpyrrolidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-((2,2,2-trifluoroethyl)amino)pyridin-4-yl)oxy)phenyl)amino)-7H-pyrrolo[2,3-d]pyrimidin-5-yl)piperidin-1-yl)prop-2-en-1-one;
1-(4-(4-((4-((2-((1H-imidazol-2-yl)amino)pyridin-4-yl)oxy)-2-fluorophenyl)amino)-7H-pyrrolo[2,3-d]pyrimidin-5-yl)piperidin-1-yl)prop-2-en-1-one;
1-(4-(4-((3-chloro-4-((4,5,6,7-tetrahydropyrazolo[1,5-a]pyridin-3-yl)methoxy)phenyl)amino)-7H-pyrrolo[2,3-d]pyrimidin-5-yl)piperidin-1-yl)prop-2-en-1-one;
1-(4-(4-((2-fluoro-4-((2-(4-fluoro-1H-pyrazol-1-yl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((4-(((1-methyl-1H-1,2,4-triazol-3-yl)amino)methyl)thiazol-2-yl)methoxy)phenyl)amino)-7H-pyrrolo[2,3-d]pyrimidin-5-yl)piperidin-1-yl)prop-2-en-1-one;
1-(4-(4-((2-fluoro-4-((2-((3aR,6aS)-hexahydrocyclopenta[c]pyrrol-2(1H)-yl)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(tetrahydro-2H-pyran-4-yl)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(pyridin-3-ylamino)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(1-methyl-1H-pyrazol-3-yl)pyridin-4-yl)oxy)phenyl)amino)-7H-pyrrolo[2,3-d]pyrimidin-5-yl)piperidin-1-yl)prop-2-en-1-one;
1-(4-(4-((4-((4H-1,2,4-triazol-3-yl)methoxy)-3-chlorophenyl)amino)-7H-pyrrolo[2,3-d]pyrimidin-5-yl)piperidin-1-yl)prop-2-en-1-one;
1-(4-(4-((3-chloro-4-((5,6,7,8-tetrahydroimidazo[1,2-a]pyridin-3-yl)methoxy)phenyl)amino)-7H-pyrrolo[2,3-d]pyrimidin-5-yl)piperidin-1-yl)prop-2-en-1-one;
1-(4-(4-((2-fluoro-4-((pyridin-2-ylamino)methyl)phenyl)amino)-7H-pyrrolo[2,3-d]pyrimidin-5-yl)piperidin-1-yl)prop-2-en-1-one;
4-((5-(1-acryloylpiperidin-4-yl)-7H-pyrrolo[2,3-d]pyrimidin-4-yl)amino)-3-fluoro-N-(pyridin-2-yl)benzamide;
1-(4-(4-((3-chloro-4-((4-methoxypyridin-3-yl)methoxy)phenyl)amino)-7H-pyrrolo[2,3-d]pyrimidin-5-yl)piperidin-1-yl)prop-2-en-1-one;
1-(4-(4-((2-fluoro-4-((2-(thiazol-2-ylamino)pyridin-4-yl)oxy)phenyl)amino)-7H-pyrrolo[2,3-d]pyrimidin-5-yl)piperidin-1-yl)prop-2-en-1-one;
1-(4-(4-((4-((1H-pyrrolo[2,3-b]pyridin-6-yl)methoxy)-3-chlorophenyl)amino)-7H-pyrrolo[2,3-d]pyrimidin-5-yl)piperidin-1-yl)prop-2-en-1-one;
1-(4-(4-((3-chloro-4-(pyridin-2-ylmethoxy)phenyl)amino)-6-(4-(4-methylpiperazin-1-yl)phenyl)-7H-pyrrolo[2,3-d]pyrimidin-5-yl)piperidin-1-yl)prop-2-en-1-one;
1-(4-(4-((3-chloro-4-(thieno[3,2-b]pyridin-5-ylmethoxy)phenyl)amino)-7H-pyrrolo[2,3-d]pyrimidin-5-yl)piperidin-1-yl)prop-2-en-1-one;
1-(4-(4-((3-chloro-4-(pyridin-2-ylmethoxy)phenyl)amino)-6-methyl-7H-pyrrolo[2,3-d]pyrimidin-5-yl)piperidin-1-yl)prop-2-en-1-one;
1-(4-(4-((3-chloro-4-(pyrimidin-5-ylmethoxy)phenyl)amino)-7H-pyrrolo[2,3-d]pyrimidin-5-yl)piperidin-1-yl)prop-2-en-1-one;
1-(4-(4-((2-fluoro-4-((2-(pyrazin-2-ylamino)pyridin-4-yl)oxy)phenyl)amino)-7H-pyrrolo[2,3-d]pyrimidin-5-yl)piperidin-1-yl)prop-2-en-1-one;
1-(4-(4-((4-((2-(3-azabicyclo[3.1.0]hexan-3-yl)pyridin-4-yl)methoxy)-2-fluorophenyl)amino)-7H-pyrrolo[2,3-d]pyrimidin-5-yl)piperidin-1-yl)prop-2-en-1-one;
1-(4-(4-((3-chloro-4-((5-methoxypyridin-3-yl)methoxy)phenyl)amino)-7H-pyrrolo[2,3-d]pyrimidin-5-yl)piperidin-1-yl)prop-2-en-1-one;
1-(4-(4-((3-chloro-4-((1-ethyl-1H-pyrazol-5-yl)methoxy)phenyl)amino)-7H-pyrrolo[2,3-d]pyrimidin-5-yl)piperidin-1-yl)prop-2-en-1-one;
1-(4-(4-((2-fluoro-4-((2-(3-fluorophenyl)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(pyrimidin-4-ylamino)pyridin-4-yl)oxy)phenyl)amino)-7H-pyrrolo[2,3-d]pyrimidin-5-yl)piperidin-1-yl)prop-2-en-1-one;
1-(4-(4-((4-((2-(cyclobutylamino)pyridin-4-yl)methoxy)-2-fluorophenyl)amino)-7H-pyrrolo[2,3-d]pyrimidin-5-yl)piperidin-1-yl)prop-2-en-1-one;
1-(4-(4-((3-chloro-4-((1-isopropyl-1H-imidazol-4-yl)methoxy)phenyl)amino)-7H-pyrrolo[2,3-d]pyrimidin-5-yl)piperidin-1-yl)prop-2-en-1-one;
1-(4-(4-((3-chloro-4-(thiazol-5-ylmethoxy)phenyl)amino)-7H-pyrrolo[2,3-d]pyrimidin-5-yl)piperidin-1-yl)prop-2-en-1-one;
1-(4-(4-((3-chloro-4-((2-(trifluoromethyl)thiazol-5-yl)methoxy)phenyl)amino)-7H-pyrrolo[2,3-d]pyrimidin-5-yl)piperidin-1-yl)prop-2-en-1-one;
1-(4-(4-((3-chloro-4-((6-cyclopropylpyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-((4-cyclopropylpyridin-2-yl)methoxy)phenyl)amino)-7H-pyrrolo[2,3-d]pyrimidin-5-yl)piperidin-1-yl)prop-2-en-1-one;
N-(4-(4-((5-(1-acryloylpiperidin-4-yl)-7H-pyrrolo[2,3-d]pyrimidin-4-yl)amino)-3-fluorophenoxy)pyridin-2-yl)cyclopentanecarboxamide;
N-(4-(4-((5-(1-acryloylpiperidin-4-yl)-7H-pyrrolo[2,3-d]pyrimidin-4-yl)amino)-3-fluorophenoxy)pyridin-2-yl)pivalamide;
1-(4-(4-((2-fluoro-4-((2-(3-methoxy-1H-pyrazol-1-yl)pyridin-4-yl)methoxy)phenyl)amino)-7H-pyrrolo[2,3-d]pyrimidin-5-yl)piperidin-1-yl)prop-2-en-1-one;
1-(4-(4-((2-fluoro-4-((2-(pyridin-2-ylamino)pyridin-4-yl)methoxy)phenyl)amino)-7H-pyrrolo[2,3-d]pyrimidin-5-yl)piperidin-1-yl)prop-2-en-1-one;
1-(4-(4-((3-chloro-4-((1-methyl-1H-imidazol-2-yl)methoxy)phenyl)amino)-7H-pyrrolo[2,3-d]pyrimidin-5-yl)piperidin-1-yl)prop-2-en-1-one;
1-(4-(4-((3-chloro-4-(oxazol-2-ylmethoxy)phenyl)amino)-7H-pyrrolo[2,3-d]pyrimidin-5-yl)piperidin-1-yl)prop-2-en-1-one;
1-(4-(4-((2-fluoro-4-((6-phenylpyridin-3-yl)methoxy)phenyl)amino)-7H-pyrrolo[2,3-d]pyrimidin-5-yl)piperidin-1-yl)prop-2-en-1-one;
1-(4-(4-((2-fluoro-4-((2-phenylpyridin-4-yl)methoxy)phenyl)amino)-7H-pyrrolo[2,3-d]pyrimidin-5-yl)piperidin-1-yl)prop-2-en-1-one;
1-(4-(4-((3-chloro-4-((6-methylpyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-((5-methoxypyridin-2-yl)methoxy)phenyl)amino)-7H-pyrrolo[2,3-d]pyrimidin-5-yl)piperidin-1-yl)prop-2-en-1-one;
2-((4-((5-(1-acryloylpiperidin-4-yl)-7H-pyrrolo[2,3-d]pyrimidin-4-yl)amino)-2-chlorophenoxy)methyl)isonicotinonitrile;
6-((4-((5-(1-acryloylpiperidin-4-yl)-7H-pyrrolo[2,3-d]pyrimidin-4-yl)amino)-2-chlorophenoxy)methyl)nicotinonitrile;
1-(4-(4-((3-chloro-4-(imidazo[1,2-a]pyridin-2-ylmethoxy)phenyl)amino)-7H-pyrrolo[2,3-d]pyrimidin-5-yl)piperidin-1-yl)prop-2-en-1-one;
1-(4-(4-((3-chloro-4-((4-(trifluoromethyl)thiazol-2-yl)methoxy)phenyl)amino)-7H-pyrrolo[2,3-d]pyrimidin-5-yl)piperidin-1-yl)prop-2-en-1-one;
1-(4-(4-((2-fluoro-4-((2-(phenylamino)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-(pyridin-2-ylmethoxy)phenyl)amino)-7H-pyrrolo[2,3-d]pyrimidin-5-yl)piperidin-1-yl)prop-2-en-1-one;
1-(4-(4-((3-chloro-2-fluoro-4-(pyridin-2-ylmethoxy)phenyl)amino)-7H-pyrrolo[2,3-d]pyrimidin-5-yl)piperidin-1-yl)prop-2-en-1-one;
1-(4-(4-((2-fluoro-4-((2-(3-(trifluoromethyl)azetidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((1-isopropyl-1H-pyrazol-4-yl)methoxy)phenyl)amino)-7H-pyrrolo[2,3-d]pyrimidin-5-yl)piperidin-1-yl)prop-2-en-1-one;
1-(4-(4-((4-((2-(cyclobutylamino)pyridin-4-yl)oxy)-2-fluorophenyl)amino)-7H-pyrrolo[2,3-d]pyrimidin-5-yl)piperidin-1-yl)prop-2-en-1-one;
1-(4-(4-((3-chloro-4-((4-methylpyridin-2-yl)methoxy)phenyl)amino)-7H-pyrrolo[2,3-d]pyrimidin-5-yl)piperidin-1-yl)prop-2-en-1-one;
1-(4-(4-((3-fluoro-4-(pyridin-2-ylmethoxy)phenyl)amino)-7H-pyrrolo[2,3-d]pyrimidin-5-yl)piperidin-1-yl)prop-2-en-1-one;
1-(4-(4-((3-chloro-4-((5-(trifluoromethyl)pyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-((4-(trifluoromethyl)pyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-(pyrimidin-4-ylmethoxy)phenyl)amino)-7H-pyrrolo[2,3-d]pyrimidin-5-yl)piperidin-1-yl)prop-2-en-1-one;
1-(4-(4-((2-fluoro-4-((2-(3-isopropoxy-1H-pyrazol-1-yl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-(pyrazin-2-ylmethoxy)phenyl)amino)-7H-pyrrolo[2,3-d]pyrimidin-5-yl)piperidin-1-yl)prop-2-en-1-one;
1-(4-(4-((3-chloro-4-((4-chloropyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-((5-fluoropyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-(thiazol-4-ylmethoxy)phenyl)amino)-7H-pyrrolo[2,3-d]pyrimidin-5-yl)piperidin-1-yl)prop-2-en-1-one;
1-(4-(4-((3-chloro-4-(pyridazin-3-ylmethoxy)phenyl)amino)-7H-pyrrolo[2,3-d]pyrimidin-5-yl)piperidin-1-yl)prop-2-en-1-one;
1-(4-(4-((3-chloro-4-(pyridin-3-ylmethoxy)phenyl)amino)-7H-pyrrolo[2,3-d]pyrimidin-5-yl)piperidin-1-yl)prop-2-en-1-one;
1-(4-(4-((2-fluoro-4-((2-(4-hydroxy-4-(trifluoromethyl)piperidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-(imidazo[1,2-b]pyridazin-6-yloxy)phenyl)amino)-7H-pyrrolo[2,3-d]pyrimidin-5-yl)piperidin-1-yl)prop-2-en-1-one;
(S)-1-(4-(4-((2-fluoro-4-((1-(phenylsulfonyl)piperidin-3-yl)oxy)phenyl)amino)-7H-pyrrolo[2,3-d]pyrimidin-5-yl)piperidin-1-yl)prop-2-en-1-one;
1-(4-(4-((2-fluoro-4-((2-(1-methyl-1H-pyrazol-4-yl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((5-chloropyridin-2-yl)methoxy)phenyl)amino)-7H-pyrrolo[2,3-d]pyrimidin-5-yl)piperidin-1-yl)prop-2-en-1-one;
1-(4-(4-((3-chloro-4-(pyridin-4-ylmethoxy)phenyl)amino)-7H-pyrrolo[2,3-d]pyrimidin-5-yl)piperidin-1-yl)prop-2-en-1-one;
1-(4-(4-((3-chloro-4-(pyrimidin-2-ylmethoxy)phenyl)amino)-7H-pyrrolo[2,3-d]pyrimidin-5-yl)piperidin-1-yl)prop-2-en-1-one;
1-(4-(4-((4-((2-(3-(dimethylamino)azetidin-1-yl)pyridin-4-yl)oxy)-2-fluorophenyl)amino)-7H-pyrrolo[2,3-d]pyrimidin-5-yl)piperidin-1-yl)prop-2-en-1-one;
1-(4-(4-((2-fluoro-4-((2-(3-hydroxy-3-isopropylazetidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((4-((2-(3-azabicyclo[3.1.0]hexan-3-yl)pyridin-4-yl)oxy)-2-fluorophenyl)amino)-7H-pyrrolo[2,3-d]pyrimidin-5-yl)piperidin-1-yl)prop-2-en-1-one;
1-(4-(4-((4-((2-((3,3-difluorocyclobutyl)(methyl)amino)pyridin-4-yl)oxy)-2-fluorophenyl)amino)-7H-pyrrolo[2,3-d]pyrimidin-5-yl)piperidin-1-yl)prop-2-en-1-one;
1-(4-(4-((2-fluoro-4-((2-(3-isopropyl-1H-pyrazol-1-yl)pyridin-4-yl)oxy)phenyl)amino)-7H-pyrrolo[2,3-d]pyrimidin-5-yl)piperidin-1-yl)prop-2-en-1-one;
N-(3-(4-((3-chloro-4-(pyridin-2-ylmethoxy)phenyl)amino)-7H-pyrrolo[2,3-d]pyrimidin-5-yl)phenyl)acrylamide;
1-(4-(4-((2-fluoro-4-((2-(pyridin-2-ylamino)pyridin-4-yl)oxy)phenyl)amino)-7H-pyrrolo[2,3-d]pyrimidin-5-yl)piperidin-1-yl)prop-2-en-1-one;
1-(4-(4-((2-fluoro-4-((2-(3-methoxy-1H-pyrazol-1-yl)pyridin-4-yl)oxy)phenyl)amino)-7H-pyrrolo[2,3-d]pyrimidin-5-yl)piperidin-1-yl)prop-2-en-1-one;
1-(3-(4-((3-chloro-4-(pyridin-2-ylmethoxy)phenyl)amino)-7H-pyrrolo[2,3-d]pyrimidin-5-yl)azetidin-1-yl)prop-2-en-1-one;
4-(4-(4-((5-(1-acryloylpiperidin-4-yl)-7H-pyrrolo[2,3-d]pyrimidin-4-yl)amino)-2-chloro-3-fluorophenoxy)phenyl)-6-methylpyridin-2(1H)-one;
1-(4-(4-((4-((2-((3R,4S)-3,4-dimethoxypyrrolidin-1-yl)pyridin-4-yl)oxy)-2-fluorophenyl)amino)-7H-pyrrolo[2,3-d]pyrimidin-5-yl)piperidin-1-yl)prop-2-en-1-one;
1-(4-(4-((2-fluoro-4-((2-(3-(trifluoromethyl)pyrrolidin-1-yl)pyridin-4-yl)oxy)phenyl)amino)-7H-pyrrolo[2,3-d]pyrimidin-5-yl)piperidin-1-yl)prop-2-en-1-one;
(S)-1-(4-(4-((2-fluoro-4-((2-(2-methylmorpholino)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-((2-((2S,6R)-2,6-dimethylmorpholino)pyridin-4-yl)oxy)-2-fluorophenyl)amino)-7H-pyrrolo[2,3-d]pyrimidin-5-yl)piperidin-1-yl)prop-2-en-1-one;
1-(4-(4-((3,4-dichloro-2-fluorophenyl)amino)-7H-pyrrolo[2,3-d]pyrimidin-5-yl)piperidin-1-yl)prop-2-en-1-one;
1-(4-(4-((2-fluoro-4-((2-(3-hydroxy-3-(trifluoromethyl)azetidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((4-((2-(3,3-difluoropyrrolidin-1-yl)pyridin-4-yl)oxy)-2-fluorophenyl)amino)-7H-pyrrolo[2,3-d]pyrimidin-5-yl)piperidin-1-yl)prop-2-en-1-one;
1-(4-(4-((2-fluoro-4-((2-((tetrahydro-2H-pyran-4-yl)amino)pyridin-4-yl)oxy)phenyl)amino)-7H-pyrrolo[2,3-d]pyrimidin-5-yl)piperidin-1-yl)prop-2-en-1-one;
(R)-1-(4-(4-((2-fluoro-4-((2-(3-methoxypiperidin-1-yl)pyridin-4-yl)oxy)phenyl)amino)-7H-pyrrolo[2,3-d]pyrimidin-5-yl)piperidin-1-yl)prop-2-en-1-one;
1-(4-(4-((4-((2-(6,6-difluoro-3-azabicyclo[3.1.0]hexan-3-yl)pyridin-4-yl)oxy)-2-fluorophenyl)amino)-7H-pyrrolo[2,3-d]pyrimidin-5-yl)piperidin-1-yl)prop-2-en-1-one;
1-(4-(4-((2-fluoro-4-((4-(2-methoxypyrimidin-5-yl)thiazol-2-yl)oxy)phenyl)amino)-7H-pyrrolo[2,3-d]pyrimidin-5-yl)piperidin-1-yl)prop-2-en-1-one;
1-(4-(4-((2-fluoro-4-((1-(5-fluoro-6-methylpyridin-3-yl)-1H-pyrazol-3-yl)oxy)phenyl)amino)-7H-pyrrolo[2,3-d]pyrimidin-5-yl)piperidin-1-yl)prop-2-en-1-one;
1-(4-(4-((2-fluoro-4-((1-((4-phenoxypyridin-2-yl)methyl)-1H-pyrazol-3-yl)oxy)phenyl)amino)-7H-pyrrolo[2,3-d]pyrimidin-5-yl)piperidin-1-yl)prop-2-en-1-one;
1-(4-(4-((2-fluoro-4-((2-(4-methylpiperazin-1-yl)pyridin-4-yl)oxy)phenyl)amino)-7H-pyrrolo[2,3-d]pyrimidin-5-yl)piperidin-1-yl)prop-2-en-1-one;
1-(4-(4-((3-chloro-4-(thiazol-2-ylmethoxy)phenyl)amino)-7H-pyrrolo[2,3-d]pyrimidin-5-yl)piperidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)-3,6-dihydropyridin-1(2H)-yl)prop-2-en-1-one;
1-(4-(4-((4-((2-((2S,6R)-2,6-dimethylmorpholino)pyridin-4-yl)oxy)-2-fluorophenyl)amino)-7H-pyrrolo[2,3-d]pyrimidin-5-yl)piperidin-1-yl)prop-2-en-1-one;
1-(4-(4-((3-chloro-4-(pyridin-2-ylmethoxy)phenyl)amino)-7H-pyrrolo[2,3-d]pyrimidin-5-yl)piperidin-1-yl)prop-2-en-1-one;
1-(4-(4-((2-fluoro-4-((1-((4-methylpyridin-2-yl)methyl)-1H-pyrazol-3-yl)oxy)phenyl)amino)-7H-pyrrolo[2,3-d]pyrimidin-5-yl)piperidin-1-yl)prop-2-en-1-one;
1-(4-(4-((2-fluoro-4-((1-((4-methylpyridin-2-yl)methyl)-1H-pyrazol-3-yl)oxy)phenyl)amino)thieno[2,3-d]pyrimidin-5-yl)piperidin-1-yl)prop-2-en-1-one;
1-(4-(4-((4-((2-((2R,6S)-2,6-dimethylmorpholino)pyridin-4-yl)oxy)-2-fluorophenyl)amino)thieno[2,3-d]pyrimidin-5-yl)piperidin-1-yl)prop-2-en-1-one;
1-(4-(4-((3-chloro-4-(pyridin-2-ylmethoxy)phenyl)amino)thieno[2,3-d]pyrimidin-5-yl)piperidin-1-yl)prop-2-en-1-one;
N-(4-(4-((5-(1-acryloylpiperidin-4-yl)thieno[2,3-d]pyrimidin-4-yl)amino)-3-fluorophenoxy)pyridin-2-yl)cyclopentanecarboxamide;
N-(4-(4-((5-(1-acryloylpiperidin-4-yl)thieno[2,3-d]pyrimidin-4-yl)amino)-3-fluorophenoxy)pyridin-2-yl)pivalamide;
1-(4-(4-((3-chloro-4-(pyridin-2-ylmethoxy)phenyl)amino)-1H-pyrazolo[3,4-d]pyrimidin-3-yl)piperazin-1-yl)prop-2-en-1-one;
1-(4-(4-((2-fluoro-4-((1-((4-methylpyridin-2-yl)methyl)-1H-pyrazol-3-yl)oxy)phenyl)amino)-1H-pyrazolo[3,4-d]pyrimidin-3-yl)piperidin-1-yl)prop-2-en-1-one;
(S)-1-(4-(4-((2-fluoro-4-((1-(phenylsulfonyl)piperidin-3-yl)oxy)phenyl)amino)-1H-pyrazolo[3,4-d]pyrimidin-3-yl)piperidin-1-yl)prop-2-en-1-one;
1-(4-(4-((3-chloro-4-(pyridin-2-ylmethoxy)phenyl)amino)-1H-pyrazolo[3,4-d]pyrimidin-3-yl)piperidin-1-yl)prop-2-en-1-one;
1-(4-(4-((4-((2-((2R,6S)-2,6-dimethylmorpholino)pyridin-4-yl)oxy)-2-fluorophenyl)amino)-1H-pyrazolo[3,4-d]pyrimidin-3-yl)piperidin-1-yl)prop-2-en-1-one;
1-(3-((4-((3-chloro-4-(pyridin-2-ylmethoxy)phenyl)amino)-1H-pyrazolo[3,4-d]pyrimidin-3-yl)amino)azetidin-1-yl)prop-2-en-1-one;
1-(4-((4-((3-chloro-4-(pyridin-2-ylmethoxy)phenyl)amino)-1H-pyrazolo[3,4-d]pyrimidin-3-yl)amino)piperidin-1-yl)prop-2-en-1-one;
5-(4-((3-(1-acryloylpiperidin-4-yl)-1H-pyrazolo[3,4-d]pyrimidin-4-yl)amino)-2-chloro-3-fluorophenoxy)-6′-phenyl-[2,4′-bipyridin]-2′(1′H)-one;
1-(4-(4-((2-fluoro-4-((2-(2-methylthiazol-4-yl)pyridin-4-yl)oxy)phenyl)amino)-1H-pyrazolo[3,4-d]pyrimidin-3-yl)piperidin-1-yl)prop-2-en-1-one;
(R)-1-(4-(4-((2-fluoro-4-((2-(2-methylmorpholino)pyridin-4-yl)oxy)phenyl)amino)-1H-pyrazolo[3,4-d]pyrimidin-3-yl)piperidin-1-yl)prop-2-en-1-one;
1-(4-(4-((2-fluoro-4-((2-(3-(trifluoromethyl)pyrrolidin-1-yl)pyridin-4-yl)oxy)phenyl)amino)-1H-pyrazolo[3,4-d]pyrimidin-3-yl)piperidin-1-yl)prop-2-en-1-one;
1-(4-(4-((2-fluoro-4-((2-((tetrahydro-2H-pyran-4-yl)amino)pyridin-4-yl)oxy)phenyl)amino)-1H-pyrazolo[3,4-d]pyrimidin-3-yl)piperidin-1-yl)prop-2-en-1-one;
1-(4-(4-((4-((2-(3,3-difluoropyrrolidin-1-yl)pyridin-4-yl)oxy)-2-fluorophenyl)amino)-1H-pyrazolo[3,4-d]pyrimidin-3-yl)piperidin-1-yl)prop-2-en-1-one;
1-(4-(4-((4-((2-((3S,4R)-3,4-dimethoxypyrrolidin-1-yl)pyridin-4-yl)oxy)-2-fluorophenyl)amino)-1H-pyrazolo[3,4-d]pyrimidin-3-yl)piperidin-1-yl)prop-2-en-1-one;
1-(4-(4-((2-fluoro-4-((2-(3-hydroxy-3-(trifluoromethyl)azetidin-1-yl)pyridin-4-yl)oxy)phenyl)amino)-1H-pyrazolo[3,4-d]pyrimidin-3-yl)piperidin-1-yl)prop-2-en-1-one;
1-(4-(4-((2-fluoro-4-((2-(3-methoxy-3-methylazetidin-1-yl)pyridin-4-yl)oxy)phenyl)amino)-1H-pyrazolo[3,4-d]pyrimidin-3-yl)piperidin-1-yl)prop-2-en-1-one;
1-(4-(4-((3-(1-acryloylpiperidin-4-yl)-1H-pyrazolo[3,4-d]pyrimidin-4-yl)amino)-3-fluorophenoxy)pyridin-2-yl)pyrrolidine-3-carbonitrile;
1-(4-(4-((4-((2-(3-azabicyclo[3.1.0]hexan-3-yl)pyridin-4-yl)oxy)-2-fluorophenyl)amino)-1H-pyrazolo[3,4-d]pyrimidin-3-yl)piperidin-1-yl)prop-2-en-1-one;
1-(4-(4-((4-((2-(7-oxa-2-azaspiro[3.5]nonan-2-yl)pyridin-4-yl)oxy)-2-fluorophenyl)amino)-1H-pyrazolo[3,4-d]pyrimidin-3-yl)piperidin-1-yl)prop-2-en-1-one;
N-(4-(4-((3-(1-acryloylpiperidin-4-yl)-1H-pyrazolo[3,4-d]pyrimidin-4-yl)amino)-3-fluorophenoxy)pyridin-2-yl)isobutyramide;
1-(4-(4-((2-fluoro-4-((1-(isopropylsulfonyl)-1H-pyrazol-3-yl)oxy)phenyl)amino)-1H-pyrazolo[3,4-d]pyrimidin-3-yl)piperidin-1-yl)prop-2-en-1-one;
4-(4-((3-(1-acryloylpiperidin-4-yl)-1H-pyrazolo[3,4-d]pyrimidin-4-yl)amino)-3-fluorophenoxy)-N-methylpicolinamide;
3-(4-((3-(1-acryloylpiperidin-4-yl)-1H-pyrazolo[3,4-d]pyrimidin-4-yl)amino)-3-fluorophenoxy)-N-(3,3-difluorocyclobutyl)benzenesulfonamide;
(S)-1-(4-(4-((2-fluoro-4-((1-(thiophen-2-ylsulfonyl)piperidin-3-yl)oxy)phenyl)amino)-1H-pyrazolo[3,4-d]pyrimidin-3-yl)piperidin-1-yl)prop-2-en-1-one;
(S)-1-(4-(4-((2-fluoro-4-((1-((6-methoxypyridin-3-yl)sulfonyl)piperidin-3-yl)oxy)phenyl)amino)-1H-pyrazolo[3,4-d]pyrimidin-3-yl)piperidin-1-yl)prop-2-en-1-one;
1-(4-(4-((2-fluoro-4-((4-(6-(trifluoromethyl)pyridin-3-yl)thiazol-2-yl)oxy)phenyl)amino)-1H-pyrazolo[3,4-d]pyrimidin-3-yl)piperidin-1-yl)prop-2-en-1-one;
1-(4-(4-((2-fluoro-4-((4-(6-methoxypyridin-3-yl)thiazol-2-yl)oxy)phenyl)amino)-1H-pyrazolo[3,4-d]pyrimidin-3-yl)piperidin-1-yl)prop-2-en-1-one;
1-(4-(4-((2-fluoro-4-((4-(5-fluoro-6-methylpyridin-3-yl)thiazol-2-yl)oxy)phenyl)amino)-1H-pyrazolo[3,4-d]pyrimidin-3-yl)piperidin-1-yl)prop-2-en-1-one;
1-(4-(4-((2-fluoro-4-((4-(1-methyl-1H-pyrazol-5-yl)thiazol-2-yl)oxy)phenyl)amino)-1H-pyrazolo[3,4-d]pyrimidin-3-yl)piperidin-1-yl)prop-2-en-1-one;
N-(4-(4-((3-(1-acryloylpiperidin-4-yl)-1H-pyrazolo[3,4-d]pyrimidin-4-yl)amino)-3-fluorophenoxy)pyridin-2-yl)pivalamide;
N-(4-(4-((3-(1-acryloylpiperidin-4-yl)-1H-pyrazolo[3,4-d]pyrimidin-4-yl)amino)-3-fluorophenoxy)pyridin-2-yl)cyclopentanecarboxamide;
N-(4-(4-((3-(1-acryloylpiperidin-4-yl)-1H-pyrazolo[3,4-d]pyrimidin-4-yl)amino)-3-fluorophenoxy)pyridin-2-yl)tetrahydro-2H-pyran-4-carboxamide;
1-(4-(4-((4-((5-chloro-1,2,3-thiadiazol-4-yl)methoxy)-2-fluorophenyl)amino)-1H-pyrazolo[3,4-d]pyrimidin-3-yl)piperidin-1-yl)prop-2-en-1-one;
N-(4-(4-((3-(1-acryloylpiperidin-4-yl)-1H-pyrazolo[3,4-d]pyrimidin-4-yl)amino)-3-fluorophenoxy)pyridin-2-yl)-1-methyl-1H-pyrazole-4-carboxamide;
1-(4-(4-((2-fluoro-4-((1-((4-methylpyridin-2-yl)methyl)-1H-pyrazol-3-yl)oxy)phenyl)amino)pyrrolo[2,1-f][1,2,4]triazin-5-yl)piperidin-1-yl)prop-2-en-1-one;
1-(4-(4-((3-chloro-4-(pyridin-2-ylmethoxy)phenyl)amino)pyrrolo[2,1-f][1,2,4]triazin-5-yl)piperidin-1-yl)prop-2-en-1-one;
1-(4-(4-((4-((2-((2R,6S)-2,6-dimethylmorpholino)pyridin-4-yl)oxy)-2-fluorophenyl)amino)pyrrolo[2,1-f][1,2,4]triazin-5-yl)piperidin-1-yl)prop-2-en-1-one;
1-(4-(4-((4-((2-(6,6-difluoro-3-azabicyclo[3.1.0]hexan-3-yl)pyridin-4-yl)oxy)-2-fluorophenyl)amino)pyrrolo[2,1-f][1,2,4]triazin-5-yl)piperidin-1-yl)prop-2-en-1-one;
1-(4-(4-((2-fluoro-4-((2-(4-methylpiperazin-1-yl)pyridin-4-yl)oxy)phenyl)amino)pyrrolo[2,1-f][1,2,4]triazin-5-yl)piperidin-1-yl)prop-2-en-1-one;
(R)-1-(4-(4-((2-fluoro-4-((2-(3-methoxypiperidin-1-yl)pyridin-4-yl)oxy)phenyl)amino)pyrrolo[2,1-f][1,2,4]triazin-5-yl)piperidin-1-yl)prop-2-en-1-one;
1-(4-(4-((2-fluoro-4-((2-(3-methoxy-3-methylazetidin-1-yl)pyridin-4-yl)oxy)phenyl)amino)pyrrolo[2,1-f][1,2,4]triazin-5-yl)piperidin-1-yl)prop-2-en-1-one;
1-(4-(4-((5-(1-acryloylpiperidin-4-yl)pyrrolo[2,1-f][1,2,4]triazin-4-yl)amino)-3-fluorophenoxy)pyridin-2-yl)pyrrolidine-3-carbonitrile;
1-(4-(4-((4-((2-(7-oxa-2-azaspiro[3.5]nonan-2-yl)pyridin-4-yl)oxy)-2-fluorophenyl)amino)pyrrolo[2,1-f][1,2,4]triazin-5-yl)piperidin-1-yl)prop-2-en-1-one;
1-(4-(4-((2-fluoro-4-((4-(5-fluoro-6-methylpyridin-3-yl)thiazol-2-yl)oxy)phenyl)amino)pyrrolo[2,1-f][1,2,4]triazin-5-yl)piperidin-1-yl)prop-2-en-1-one;
1-(4-(4-((3-chloro-4-((5-fluorothiazol-2-yl)methoxy)phenyl)amino)-7H-pyrrolo[2,3-d]pyrimidin-5-yl)piperidin-1-yl)prop-2-en-1-one;
1-(4-(4-((3-bromo-2-fluoro-4-(pyridin-2-ylmethoxy)phenyl)amino)-7H-pyrrolo[2,3-d]pyrimidin-5-yl)piperidin-1-yl)prop-2-en-1-one;
1-(4-(4-((3-chloro-4-((5-methylthiazol-2-yl)methoxy)phenyl)amino)-7H-pyrrolo[2,3-d]pyrimidin-5-yl)piperidin-1-yl)prop-2-en-1-one;
1-(4-(4-((3-chloro-4-((4-methylthiazol-2-yl)methoxy)phenyl)amino)-7H-pyrrolo[2,3-d]pyrimidin-5-yl)piperidin-1-yl)prop-2-en-1-one;
1-(4-(4-((3-chloro-4-((4-fluoro-5-methylthiazol-2-yl)methoxy)phenyl)amino)-7H-pyrrolo[2,3-d]pyrimidin-5-yl)piperidin-1-yl)prop-2-en-1-one;
1-(4-(4-((3-chloro-4-((2-methylthiazol-4-yl)methoxy)phenyl)amino)-7H-pyrrolo[2,3-d]pyrimidin-5-yl)piperidin-1-yl)prop-2-en-1-one;
1-(4-(4-((3-chloro-4-((5-methylthiazol-4-yl)methoxy)phenyl)amino)-7H-pyrrolo[2,3-d]pyrimidin-5-yl)piperidin-1-yl)prop-2-en-1-one;
1-(4-(4-((3-chloro-4-((5-fluorothiazol-4-yl)methoxy)phenyl)amino)-7H-pyrrolo[2,3-d]pyrimidin-5-yl)piperidin-1-yl)prop-2-en-1-one;
1-(4-(4-((2-fluoro-4-((4-(1-methyl-1H-pyrazol-5-yl)thiazol-2-yl)oxy)phenyl)amino)pyrrolo[2,1-f][1,2,4]triazin-5-yl)piperidin-1-yl)prop-2-en-1-one;
1-(4-(4-((3-chloro-2-fluoro-4-(thiazol-4-ylmethoxy)phenyl)amino)-7H-pyrrolo[2,3-d]pyrimidin-5-yl)piperidin-1-yl)prop-2-en-1-one;
1-(4-(4-((3-chloro-4-((2-methylthiazol-4-yl)methoxy)phenyl)amino)-7H-pyrrolo[2,3-d]pyrimidin-5-yl)piperidin-1-yl)prop-2-en-1-one;
(R)-1-(4-(4-((3-chloro-4-(1-(thiazol-4-yl)ethoxy)phenyl)amino)-7H-pyrrolo[2,3-d]pyrimidin-5-yl)piperidin-1-yl)prop-2-en-1-one;
1-(4-(4-((3-chloro-4-((5-methylthiazol-4-yl)methoxy)phenyl)amino)-7H-pyrrolo[2,3-d]pyrimidin-5-yl)piperidin-1-yl)prop-2-en-1-one;
1-(4-(4-((3-bromo-2-fluoro-4-(pyridin-2-ylmethoxy)phenyl)amino)-7H-pyrrolo[2,3-d]pyrimidin-5-yl)piperidin-1-yl)prop-2-en-1-one;
1-(4-(4-((5-chloro-2-fluoro-4-(pyridin-2-ylmethoxy)phenyl)amino)-7H-pyrrolo[2,3-d]pyrimidin-5-yl)piperidin-1-yl)prop-2-en-1-one;
1-(4-(4-((2,5-dichloro-4-(pyridin-2-ylmethoxy)phenyl)amino)-7H-pyrrolo[2,3-d]pyrimidin-5-yl)piperidin-1-yl)prop-2-en-1-one;
1-(4-(4-((3-chloro-4-((5-fluorothiazol-2-yl)methoxy)phenyl)amino)-7H-pyrrolo[2,3-d]pyrimidin-5-yl)piperidin-1-yl)prop-2-en-1-one;
1-(4-(4-((3-chloro-4-(thieno[3,2-c]pyridin-6-ylmethoxy)phenyl)amino)-7H-pyrrolo[2,3-d]pyrimidin-5-yl)piperidin-1-yl)prop-2-en-1-one;
1-(4-(4-((3-chloro-4-(3-(trifluoromethyl)phenoxy)phenyl)amino)-7H-pyrrolo[2,3-d]pyrimidin-5-yl)piperidin-1-yl)prop-2-en-1-one;
1-(4-(4-((1-(3-fluorobenzyl)-1H-indazol-5-yl)amino)-7H-pyrrolo[2,3-d]pyrimidin-5-yl)piperidin-1-yl)prop-2-en-1-one;
1-(4-(4-((3-chloro-4-(pyridin-2-ylmethoxy-d2)phenyl)amino)-7H-pyrrolo[2,3-d]pyrimidin-5-yl)piperidin-1-yl)prop-2-en-1-one;
1-(4-(4-((3-chloro-4-((3-fluorobenzyl)oxy)phenyl)amino)-7H-pyrrolo[2,3-d]pyrimidin-5-yl)piperidin-1-yl)prop-2-en-1-one;
1-(4-(4-((3-chloro-4-(isothiazol-3-ylmethoxy)phenyl)amino)-7H-pyrrolo[2,3-d]pyrimidin-5-yl)piperidin-1-yl)prop-2-en-1-one;
1-((1R,3r,5S)-3-(4-((3-chloro-4-(pyridin-2-ylmethoxy)phenyl)amino)-7H-pyrrolo[2,3-d]pyrimidin-5-yl)-8-azabicyclo[3.2.1]octan-8-yl)prop-2-en-1-one;
N-(4-(4-((5-(1-acryloylpiperidin-4-yl)-7H-pyrrolo[2,3-d]pyrimidin-4-yl)amino)-3-fluorophenoxy)pyridin-2-yl)cyclobutanecarboxamide;
N-(4-(4-((5-(1-acryloylpiperidin-4-yl)-7H-pyrrolo[2,3-d]pyrimidin-4-yl)amino)-3-fluorophenoxy)pyridin-2-yl)cyclopropanecarboxamide;
N-(4-(4-((5-(1-acryloylpiperidin-4-yl)-7H-pyrrolo[2,3-d]pyrimidin-4-yl)amino)-2-chloro-3-fluorophenoxy)pyridin-2-yl)cyclopropanecarboxamide;
N-(4-(4-((5-(1-acryloylpiperidin-4-yl)-7H-pyrrolo[2,3-d]pyrimidin-4-yl)amino)-2-chlorophenoxy)pyridin-2-yl)cyclopropanecarboxamide;
N-(4-(4-((5-(1-acryloylpiperidin-4-yl)-7H-pyrrolo[2,3-d]pyrimidin-4-yl)amino)-2-chloro-3-fluorophenoxy)pyridin-2-yl)cyclobutanecarboxamide;
N-(4-(4-((5-(1-acryloylpiperidin-4-yl)-7H-pyrrolo[2,3-d]pyrimidin-4-yl)amino)-2-chlorophenoxy)pyridin-2-yl)cyclobutanecarboxamide; and
1-(4-(4-((3-chloro-4-(3-(difluoromethyl)phenoxy)phenyl)amino)-7H-pyrrolo[2,3-d]pyrimidin-5-yl)piperidin-1-yl)prop-2-en-1-one.

16. A pharmaceutical composition comprising the compound according to claim 1 and at least one additional component chosen from pharmaceutically acceptable carriers, pharmaceutically acceptable vehicles, and pharmaceutically acceptable excipients.

17. The pharmaceutical composition according to claim 16, wherein the compound is present in a therapeutically effective amount.

18. A method of treating cancer in a subject in need thereof, comprising administering to the subject an effective amount of the compound according to claim 1 or of the pharmaceutical composition according to claim 16.

19. The method according to claim 18, wherein the cancer is associated with an EGFR or HER2 exon 20 insertion mutation.

20. The method according to claim 18, wherein the cancer is chosen from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, and esophageal cancer.

21. The method according to claim 20, wherein the cancer is lung cancer.

22. The method according to claim 20, wherein the cancer is non-small cell lung cancer.

23. The method according to claim 20, wherein the cancer is pancreatic cancer.

24. The method according to claim 20, wherein the cancer is colon cancer.

25. The method according to claim 20, wherein the cancer is breast cancer.

26. The method according to claim 20, wherein the cancer is head and neck cancer.

27. The method according to claim 20, wherein the cancer is sinonasal squamous cell carcinoma.

28. (canceled)

29. The method according to claim 18, further comprising administering to the subject in combination with another agent.

30. The method according to claim 29, wherein the agent is chosen from gemcitabine, cisplatin, erlotinib, gefitinib, pemetrexed, bevacizumab, cetuximab, trastuzumab, pertuzumab, sorafenib, lapatinib, cobimetinib, selumetinib, and everolimus.

31. The method according to claim 29, wherein the compound according to claim 1 and the additional agent are administered concomitantly.

32. The method according to claim 29, wherein the compound according to claim 1 and the additional agent are administered sequentially.

33. The method according to claim 29, wherein the pharmaceutical composition of claim 16 and the additional agent are administered concomitantly.

34. The method according to claim 29, wherein the pharmaceutical composition of claim 16 and the additional agent are administered sequentially.
